SEQUENCE LISTING (105 sequences)

SEQ ID NO: 1 - DNA, 15186 bp, Synthetic construct
  source 1..15186: mol_type = other DNA; note = cDNA of genomic sequence of NDV strain LaSota
accaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggccgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactacgtacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcggacccaaggtccaactctccaagcggcaatcctctctcgcttcctcagccccactgaatgatcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccaccgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagagggatgtctacaagatcccagctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccactgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcaccggtaaccctattctatacgactcacagactcaactcttgggtatacaggtaactctaccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggttaacaatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctggacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagcgacctgacctcagataaagtagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcgaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagttatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggt

SEQ ID NO: 2 - DNA, 15186 bp, Synthetic construct
  source 1..15186: mol_type = other DNA; note = cDNA of genomic sequence of NDV strain Hitchner B1
accaaacagagaatccgtaagttacgataaaaggcgaaggagcaattgaagtcgcacgggtagaaggtgtgaatctcgagtgcgagcccgaagcacaaactcgaggaagccttctgccaacatgtcttccgtattcgacgagtacgaacagctcctcgcggctcagactcgccccaatggagctcatggagggggggagaaagggagtaccttaaaagtagacgtcccggtattcactcttaacagtgatgacccagaagataggtggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggcaccccgttcgtcacagccggggctgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctagtaggggacgtagactcatatatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccggcagcaaggaggggcctggcagctgctgcccaacgagtctccgaggtgaccagcagcatagacatgcctactcaacaagtcggagtcctcactgggcttagcgaggggggatcccaagccctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgattgacaaaacccagcctgcttctacaagaacatcccaatgctctcacccgtagtcgacccctcgatttgcggctctatatgaccacaccctcaaacaaacatccccctctttcctccctccccctgctgtacaactccgcacgccctagataccacaggcacaccgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacagggcaagaccaaggtgctgagcgcagcatgggagaagcatgggagcatccagccaccggccagtcaagacaacctcgatcgacaggacagatctgacaaacaaccatccacacccgagcaaacgaccccgcacgacagcccgccggccacatccgctgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagccaggaaagactgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatgggggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccatctccctatgtgatacaaggaggcgaaatggcacttaataaactttcgcaaccagtgccacatccatctgaattgattaaacccgccactgcatgcgggcctgatataggagtggagagggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcagacccaaggtccaactctagaagcggcaatcctctctcgcttcctcagccccactgaatgatcgcgtaaccgtaattaatctagctacattaaggattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgactcgtggactgatagtaaggaagactcagtattcatcaccacctatggattcatctttcaagttgggaatgaggaagccactgtcggcatgatcgatgataaacccaagcgcgagttactttccgctgcgatgctctgcctaggaagcgtcccaaataccggagaccttgttgagctggcaagggcctgtctcactatgatggtcacatgcaagaagagtgcaactaatactgagagaatggttttctcagtagtgcaggcaccccaagtgctgcaaagctgtagggttgtggcaaataaatactcatcagtgaatgcagtcaagcacgtgaaagcgccagagaagatccccgggagtggaaccctagaatacaaggtgaactttgtctccttgactgtggtaccgaagaaggatgtctacaagatcccagctgcagtattgaagatttctggctcgagtctgtacaatcttgcgctcaatgtcactattaatgtggaggtagacccgaggagtcctttggttaaatctctgtctaagtctgacagcggatactatgctaacctcttcttgcatattggacttatgaccaccgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgctgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtccatcaagttagaaaaaacacgggtagaagattctggatcccggttggcgccctccaggtgcaggatgggctccagaccttctaccaagaacccagcacctatgatgctgactatccgggtcgcgctggtactgagttgcatctgcccggcaaactccattgatggcaggcctcttgcagctgcaggaattgtggttacaggagacaaagcagtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgccttaaacaagctgactattcaggcactttacaatctagctggtgggaatatggattacttattgactaagttaggtatagggaacaatcaactcagctcattaatcggtagcggcttaatcaccggtaaccctattctatacgactcacagactcaactcttgggtatacaggtaactctaccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttactcctgcttgagcggcaatacatcggcctgtatgtactcaaagaccgaaggcgcacttactacaccatatatgactatcaaaggctcagtcatcgctaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgaccagcacatctgctctcattacctatatcgttttgactatcatatctcttgtttttggtatacttagcctgattctagcatgctacctaatgtacaagcaaaaggcgcaacaaaagaccttattatggcttgggaataataccctagatcagatgagagccactacaaaaatgtgaacacagatgaggaacgaaggtttccctaatagtaatttgtgtgaaagttctggtagtctgtcagttcggagagttaagaaaaaactaccggttgtagatgaccaaaggacgatatacgggtagaacggtaagagaggccgcccctcaattgcgagccagacttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccattggcattgttaaatactgagaccacaattatgaacgcaataacatctctctcttatcagattaatggagctgcaaacaacagcgggtggggggcacctattcatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacactcacatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagccacggagacagaggaagaagattataactcagctgtccctacgcggatggtacatgggaggttagggttcgacggccaatatcacgaaaaggacctagatgtcacaacattattcggggactgggtggccaactacccaggagtagggggtggatcttttattgacagccgcgtatggttctcagtctacggagggttaaaacccaatacacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaagtgtcaacatccttaggcgaagacccggtactgactgtaccgcccaacacagtcacactcatgggggccgaaggcagaattctcacagtagggacatcccatttcttgtatcagcgagggtcatcatacttctctcccgcgttattatatcctatgacagtcagcgacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgaacaagcaagacttaaccctgcgtctgcagtattcgatagcacatcccgcagtcgcataactcgagtgagttcaagcagcatcaaagcagcatacacaacatcaacttgttttaaagtggtcaagaccaataagacctattgtctcagcattgctgaaatatctaatactctcttcggagaattcagaatcgtcccgttactagttgagatcctcaaagatgacggggttagagaagccaggtctggctagttgagtcaactatgaaagagttggaaagatggcattgtatcacctatcttctgcgacatcaagaatcaaaccgaatgccggcgcgtgctcgaattccatgtcgccagttgaccacaatcagccagtgctcatgcgatcagattaagccttgtcaatagtctcttgattaagaaaaaatgtaagtggcaatgagatacaaggcaaaacagctcacggtaaataatacgggtaggacatggcgagctccggtcctgaaagggcagagcatcagattatcctaccagagtcacacctgtcttcaccattggtcaagcacaaactactctattattggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtactccaccccaggtgtttagaagaactggctaatattgaggtccctgattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgattgtggcagctaggacaaggtctgcggccaacaaattggtgatgctaacccataaggtaggccaagtctttgtcactcctgaacttgttgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcgggagatttcttcgcattcaacctgcaggagcttaaagacattctaattggcctcctccccaatgatatagcagaatccgtgactcatgcaatcgctactgtattctctggtttagaacagaatcaagcagctgagatgttgtgcctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacggatacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatacgaccctgtcactaacctgagcatgttcctaaaagacaaggcaatcgcacaccccaacgataattggcttgcctcgtttaggcggaaccttctctccgaagaccagaagaaacatgtaaaggaagcgacttcgactaaccgcctcttgatagagtttttagagtcaaatgattttgatccatataaagagatggaatatctgacgacccttgagtaccttagagatgacaatgtggcagtatcatactcgctcaaagagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatgttcaaaccgcaatcatgatccgaaaagcaagaaccgtcggagagttgcaaccttcataacaactgacctgcaaaagtactgtcttaattggagatatcagacgatcaaattgttcgctcatgccatcaatcagttgatgggcctacctcatttcttcgagtggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagatgactctccggagatggtgttgacacagttgcatcaagccagtgataatttcttcaaggaattaatccatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggactactgagtcctaacattaggactaatatcttaactaggccgcctgggaatggagattgggccagtctgtgcaacgacccatactctttcaattttgagactgttgcaagcccaaacattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacactgtaattaagattgcgcttactaggaggccattaggcatcaagaggctgatgcggatagtcaattattctagcatgcatgcaatgctgtttagagacgatgttttttcctctagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcgaaaatagctcatatgtcgccacatgtgaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccagaacatatgatgagatcacactgcacctacatagtaaatttagttgctgtatcagggaagcacctgttgcggttcctttcgagctacttggggtggcaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgatgccataatagtgtatgacaatacccgaaattggatcagtgaagctcagaattcagatgtggtccgcctatttgaatatgcagcacttgaagtgctcctcgactgttcttaccaactctattacctgagagtaagagacctagacaatattgtcttatatatgggtgatttatacaagaatatgccaggaattctactttccaacattgcagctacaatatctcatcctgtcattcattcaaggttacatgcagtgggcctggtcaaccatgacggatcacaccaacttgcagatacggattttatcgaaatgtctgcaaaactgttagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatcccatctcctttctgtacccgaggtaagatgtgcaagacacgggaactccttatacttggctgaaggaagcggagccatcatgagtcttcttgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaggatggatttgtccaagagttccgtccattatggagagaaaatacagaggaaagtgacctgacctcagataaagcagtggggtatattacatctgcagtaccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggggatatggagtgttacctggtatttgtcatgggttacctgggcgggcctacatttgtacatgaggtggtgaggatggcaaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagtcatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcctaacgaaaatcacatattaataggctccttttttggccaattgtattcttgttgatttaattatattatgttagaaaaaagttgaactctgactccttaggactcgaattcgaactcaaataaatgtctttaaaaaaggttgcgcacaattattcttgagtgtagtctcgtcattcaccaaatctttgtttggt

SEQ ID NO: 3 - DNA, 15186 bp, Synthetic construct
  source 1..15186: mol_type = other DNA; note = cDNA of genomic sequence of NDV strain LaSota (L289A mutation)
accaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggccgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactacgtacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcggacccaaggtccaactctccaagcggcaatcctctctcgcttcctcagccccactgaatgatcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccaccgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagagggatgtctacaagatcccagctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccactgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcaccggtaaccctattctatacgactcacagactcaactcttgggtatacaggtaactgccccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggttaacaatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctggacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagcgacctgacctcagataaagtagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcgaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagttatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggt

SEQ ID NO: 4 - protein, 553 aa, Synthetic construct
  source 1..553: note = Amino acid sequence of F protein of NDV strain LaSota
MGSRPSTKNPAPMTLTIRVALVLSCICPANSIDGRPLAAAGIVVTGDKAVNIYTSSQTGSIIVKLLPNLPKDKEACAKAPLDAYNRTLTTLLTPLGDSIRRIQESVTTSGGGRQGRLIGAIIGGVALGVATAAQITAAAALIQAKQNAANILRLKESIAATNEAVHEVTDGLSQLAVAVGKMQQFVNDQFNKTAQELDCIKIAQQVGVELNLYLTELTTVFGPQITSPALNKLTIQALYNLAGGNMDYLLTKLGVGNNQLSSLIGSGLITGNPILYDSQTQLLGIQVTLPSVGNLNNMRATYLETLSVSTTRGFASALVPKVVTQVGSVIEELDTSYCIETDLDLYCTRIVTFPMSPGIYSCLSGNTSACMYSKTEGALTTPYMTIKGSVIANCKMTTCRCVNPPGIISQNYGEAVSLIDKQSCNVLSLGGITLRLSGEFDVTYQKNISIQDSQVIITGNLDISTELGNVNNSISNALNKLEESNRKLDKVNVKLTSTSALITYIVLTIISLVFGILSLILACYLMYKQKAQQKTLLWLGNNTLDQMRATTKM

SEQ ID NO: 5 - protein, 53 aa, Synthetic construct
  source 1..53: note = Amino acid sequence of transmembrane and cytoplasmic domains of F protein of NDV strain LaSota
LITYIVLTIISLVFGILSLILACYLMYKQKAQQKTLLWLGNNTLDQMRATTKM

SEQ ID NO: 6 - DNA, 3798 bp, synthetic construct
  source 1..3798: mol_type = other DNA; note = Nucleotide sequence of Omicron BA.1
atgttcgtgtttctggtgctgctgcctctggtgtccagccagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgtgatctccggcaccaatggcaccaagagattcgacaaccccgtgctgcccttcaacgacggggtgtactttgccagcatcgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcatcgtgcgggaacccgaagatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacctggcccctttcttcaccttcaagtgctacggcgtgtcccctaccaagctgaacgacctgtgcttcacaaacgtgtacgccgacagcttcgtgatccggggagatgaagtgcggcagattgcccctggacagacaggcaacatcgccgactacaactacaagctgcccgacgacttcaccggctgtgtgattgcctggaacagcaacaagctggactccaaagtcagcggcaactacaattacctgtaccggctgttccggaagtccaatctgaagcccttcgagcgggacatctccaccgagatctatcaggccggcaacaagccttgtaacggcgtggccggcttcaactgctacttcccactgagatcctacagctttagacccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaagggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaagggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcttcagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtga

SEQ ID NO: 7 - DNA, 3759 bp, synthetic construct
  source 1..3759: mol_type = other DNA; note = Nucleotide sequence of Omicron BA.1
cagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgtgatctccggcaccaatggcaccaagagattcgacaaccccgtgctgcccttcaacgacggggtgtactttgccagcatcgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcatcgtgcgggaacccgaagatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacctggcccctttcttcaccttcaagtgctacggcgtgtcccctaccaagctgaacgacctgtgcttcacaaacgtgtacgccgacagcttcgtgatccggggagatgaagtgcggcagattgcccctggacagacaggcaacatcgccgactacaactacaagctgcccgacgacttcaccggctgtgtgattgcctggaacagcaacaagctggactccaaagtcagcggcaactacaattacctgtaccggctgttccggaagtccaatctgaagcccttcgagcgggacatctccaccgagatctatcaggccggcaacaagccttgtaacggcgtggccggcttcaactgctacttcccactgagatcctacagctttagacccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaagggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaagggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcttcagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtga

SEQ ID NO: 8 - protein, 1265 aa, synthetic construct
  source 1..1265: note = Amino acid sequence of Omicron BA.1
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFKGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 9 - protein, 1252 aa, synthetic construct
  source 1..1252: note = Amino acid sequence of Omicron BA.1
QCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFKGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 10 - DNA, 3783 bp, synthetic construct
  source 1..3783: mol_type = other DNA; note = Nucleotide sequence of Omicron BA.1 (H655_deCSV687I)
atgttcgtgtttctggtgctgctgcctctggtgtccagccagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgtgatctccggcaccaatggcaccaagagattcgacaaccccgtgctgcccttcaacgacggggtgtactttgccagcatcgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcatcgtgcgggaacccgaagatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacctggcccctttcttcaccttcaagtgctacggcgtgtcccctaccaagctgaacgacctgtgcttcacaaacgtgtacgccgacagcttcgtgatccggggagatgaagtgcggcagattgcccctggacagacaggcaacatcgccgactacaactacaagctgcccgacgacttcaccggctgtgtgattgcctggaacagcaacaagctggactccaaagtcagcggcaactacaattacctgtaccggctgttccggaagtccaatctgaagcccttcgagcgggacatctccaccgagatctatcaggccggcaacaagccttgtaacggcgtggccggcttcaactgctacttcccactgagatcctacagctttagacccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaagggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaatcgccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaagggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcttcagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtga

SEQ ID NO: 11 - DNA, 3744 bp, synthetic construct
  source 1..3744: mol_type = other DNA; note = Nucleotide sequence of Omicron BA.1 (H655_deCSV687I)
cagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgtgatctccggcaccaatggcaccaagagattcgacaaccccgtgctgcccttcaacgacggggtgtactttgccagcatcgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcatcgtgcgggaacccgaagatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacctggcccctttcttcaccttcaagtgctacggcgtgtcccctaccaagctgaacgacctgtgcttcacaaacgtgtacgccgacagcttcgtgatccggggagatgaagtgcggcagattgcccctggacagacaggcaacatcgccgactacaactacaagctgcccgacgacttcaccggctgtgtgattgcctggaacagcaacaagctggactccaaagtcagcggcaactacaattacctgtaccggctgttccggaagtccaatctgaagcccttcgagcgggacatctccaccgagatctatcaggccggcaacaagccttgtaacggcgtggccggcttcaactgctacttcccactgagatcctacagctttagacccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaagggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaatcgccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaagggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcttcagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtga

SEQ ID NO: 12 - protein, 1260 aa, synthetic construct
  source 1..1260: note = Amino acid sequence of Omicron BA.1 (H655_deCSV687I)
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTIASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFKGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 13 - protein, 1247 aa, synthetic construct
  source 1..1247: note = Amino acid sequence of Omicron BA.1 (H655_deCSV687I)
QCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTIASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFKGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 14 - DNA, 3798 bp, synthetic construct
  source 1..3798: mol_type = other DNA; note = Nucleotide sequence of Omicron BA.1 (Q493Q498)
atgttcgtgtttctggtgctgctgcctctggtgtccagccagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgtgatctccggcaccaatggcaccaagagattcgacaaccccgtgctgcccttcaacgacggggtgtactttgccagcatcgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcatcgtgcgggaacccgaagatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacctggcccctttcttcaccttcaagtgctacggcgtgtcccctaccaagctgaacgacctgtgcttcacaaacgtgtacgccgacagcttcgtgatccggggagatgaagtgcggcagattgcccctggacagacaggcaacatcgccgactacaactacaagctgcccgacgacttcaccggctgtgtgattgcctggaacagcaacaagctggactccaaagtcagcggcaactacaattacctgtaccggctgttccggaagtccaatctgaagcccttcgagcgggacatctccaccgagatctatcaggccggcaacaagccttgtaacggcgtggccggcttcaactgctacttcccactgcagtcctacagctttcagcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaagggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaagggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcttcagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtga

SEQ ID NO: 15 - DNA, 3759 bp, synthetic construct
  source 1..3759: mol_type = other DNA; note = Nucleotide sequence of Omicron BA.1 (Q493Q498)
cagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgtgatctccggcaccaatggcaccaagagattcgacaaccccgtgctgcccttcaacgacggggtgtactttgccagcatcgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcatcgtgcgggaacccgaagatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacctggcccctttcttcaccttcaagtgctacggcgtgtcccctaccaagctgaacgacctgtgcttcacaaacgtgtacgccgacagcttcgtgatccggggagatgaagtgcggcagattgcccctggacagacaggcaacatcgccgactacaactacaagctgcccgacgacttcaccggctgtgtgattgcctggaacagcaacaagctggactccaaagtcagcggcaactacaattacctgtaccggctgttccggaagtccaatctgaagcccttcgagcgggacatctccaccgagatctatcaggccggcaacaagccttgtaacggcgtggccggcttcaactgctacttcccactgcagtcctacagctttcagcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaagggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaagggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcttcagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtga

SEQ ID NO: 16 - protein, 1265 aa, synthetic construct
  source 1..1265: note = Amino acid sequence of Omicron BA.1 (Q493Q498)
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLQSYSFQPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFKGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 17 - protein, 1252 aa, synthetic construct
  source 1..1252: note = Amino acid sequence of Omicron BA.1 (Q493Q498)
QCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLQSYSFQPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFKGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 18 - DNA, 3582 bp, synthetic construct
  source 1..3582: mol_type = other DNA; note = Nucleotide sequence of Omicron BA.1 Spike Protein Ectodomain
cagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgtgatctccggcaccaatggcaccaagagattcgacaaccccgtgctgcccttcaacgacggggtgtactttgccagcatcgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcatcgtgcgggaacccgaagatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacctggcccctttcttcaccttcaagtgctacggcgtgtcccctaccaagctgaacgacctgtgcttcacaaacgtgtacgccgacagcttcgtgatccggggagatgaagtgcggcagattgcccctggacagacaggcaacatcgccgactacaactacaagctgcccgacgacttcaccggctgtgtgattgcctggaacagcaacaagctggactccaaagtcagcggcaactacaattacctgtaccggctgttccggaagtccaatctgaagcccttcgagcgggacatctccaccgagatctatcaggccggcaacaagccttgtaacggcgtggccggcttcaactgctacttcccactgagatcctacagctttagacccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaagggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaagggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcttcagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 19 - protein, 1194 aa, synthetic construct
  source 1..1194: note = Amino acid sequence of Omicron BA.1 Spike Protein Ectodomain
QCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFKGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 20 - DNA, 3567 bp, synthetic construct
  source 1..3567: mol_type = other DNA; note = Nucleotide sequence of Omicron BA.1 (H655_deCSV687I) Spike Protein Ectodomain
cagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgtgatctccggcaccaatggcaccaagagattcgacaaccccgtgctgcccttcaacgacggggtgtactttgccagcatcgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcatcgtgcgggaacccgaagatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacctggcccctttcttcaccttcaagtgctacggcgtgtcccctaccaagctgaacgacctgtgcttcacaaacgtgtacgccgacagcttcgtgatccggggagatgaagtgcggcagattgcccctggacagacaggcaacatcgccgactacaactacaagctgcccgacgacttcaccggctgtgtgattgcctggaacagcaacaagctggactccaaagtcagcggcaactacaattacctgtaccggctgttccggaagtccaatctgaagcccttcgagcgggacatctccaccgagatctatcaggccggcaacaagccttgtaacggcgtggccggcttcaactgctacttcccactgagatcctacagctttagacccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaagggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaatcgccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaagggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcttcagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 21 - protein, 1189 aa, synthetic construct
  source 1..1189: note = Amino acid sequence of Omicron BA.1 (H655_deCSV687I) Spike Protein Ectodomain
QCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTIASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFKGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 22 - DNA, 3582 bp, synthetic construct
  source 1..3582: mol_type = other DNA; note = Nucleotide sequence of Omicron BA.1 (Q493Q498) Spike Protein Ectodomain
cagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgtgatctccggcaccaatggcaccaagagattcgacaaccccgtgctgcccttcaacgacggggtgtactttgccagcatcgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcatcgtgcgggaacccgaagatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacctggcccctttcttcaccttcaagtgctacggcgtgtcccctaccaagctgaacgacctgtgcttcacaaacgtgtacgccgacagcttcgtgatccggggagatgaagtgcggcagattgcccctggacagacaggcaacatcgccgactacaactacaagctgcccgacgacttcaccggctgtgtgattgcctggaacagcaacaagctggactccaaagtcagcggcaactacaattacctgtaccggctgttccggaagtccaatctgaagcccttcgagcgggacatctccaccgagatctatcaggccggcaacaagccttgtaacggcgtggccggcttcaactgctacttcccactgcagtcctacagctttcagcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaagggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaagggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcttcagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 23 - protein, 1194 aa, synthetic construct
  source 1..1194: note = Amino acid sequence of Omicron BA.1 (Q493Q498) Spike Protein Ectodomain
QCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLQSYSFQPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFKGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 24 - protein, 5 aa, synthetic construct
  source 1..5: note = Linker Sequence
GGGGS

SEQ ID NO: 25 - skipped

SEQ ID NO: 26 - DNA, 10 bp, synthetic construct
  source 1..10: mol_type = other DNA; note = NDV Gene End Sequence
ttagaaaaaa

SEQ ID NO: 27 - DNA, 10 bp, synthetic construct
  source 1..10: mol_type = other DNA; note = NDV Gene Start Sequence
acgggtagaa

SEQ ID NO: 28 - skipped

SEQ ID NO: 29 - protein, 13 aa, synthetic construct
  source 1..13: note = Signal Sequence
MFVFLVLLPLVSS

SEQ ID NO: 30 - DNA, 3798 bp, synthetic construct
  source 1..3798: mol_type = other DNA; note = Nucleotide Sequence of BA.1 S371 S373 S375
atgttcgtgtttctggtgctgctgcctctggtgtccagccagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgtgatctccggcaccaatggcaccaagagattcgacaaccccgtgctgcccttcaacgacggggtgtactttgccagcatcgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcatcgtgcgggaacccgaagatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaacatcgccgactacaactacaagctgcccgacgacttcaccggctgtgtgattgcctggaacagcaacaagctggactccaaagtcagcggcaactacaattacctgtaccggctgttccggaagtccaatctgaagcccttcgagcgggacatctccaccgagatctatcaggccggcaacaagccttgtaacggcgtggccggcttcaactgctacttcccactgagatcctacagctttagacccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaagggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaagggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcttcagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtga

SEQ ID NO: 31 - protein, 1265 aa, synthetic construct
  source 1..1265: note = Amino Acid Sequence of BA.1 S371 S373 S375
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFKGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 32 - DNA, 3621 bp, synthetic construct
  source 1..3621: mol_type = other DNA; note = Nucleotide Sequence of BA.1 S371 S373 S375
atgttcgtgtttctggtgctgctgcctctggtgtccagccagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgtgatctccggcaccaatggcaccaagagattcgacaaccccgtgctgcccttcaacgacggggtgtactttgccagcatcgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcatcgtgcgggaacccgaagatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaacatcgccgactacaactacaagctgcccgacgacttcaccggctgtgtgattgcctggaacagcaacaagctggactccaaagtcagcggcaactacaattacctgtaccggctgttccggaagtccaatctgaagcccttcgagcgggacatctccaccgagatctatcaggccggcaacaagccttgtaacggcgtggccggcttcaactgctacttcccactgagatcctacagctttagacccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaagggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaagggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcttcagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 33 - protein, 1207 aa, synthetic construct
  source 1..1207: note = Amino Acid Sequence of BA.1 S371 S373 S375
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFKGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 34 - DNA, 3582 bp, synthetic construct
  source 1..3582: mol_type = other DNA; note = Nucleotide Sequence of BA.1 S371 S373 S375
cagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgtgatctccggcaccaatggcaccaagagattcgacaaccccgtgctgcccttcaacgacggggtgtactttgccagcatcgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcatcgtgcgggaacccgaagatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaacatcgccgactacaactacaagctgcccgacgacttcaccggctgtgtgattgcctggaacagcaacaagctggactccaaagtcagcggcaactacaattacctgtaccggctgttccggaagtccaatctgaagcccttcgagcgggacatctccaccgagatctatcaggccggcaacaagccttgtaacggcgtggccggcttcaactgctacttcccactgagatcctacagctttagacccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaagggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaagggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcttcagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 35 - protein, 1194 aa, synthetic construct
  source 1..1194: note = Amino Acid Sequence of BA.1 S371 S373 S375
QCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFKGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 36 - DNA, 3798 bp, synthetic construct
  source 1..3798: mol_type = other DNA; note = Nucleotide Sequence of BA.2
atgttcgtgtttctggtgctgctgcctctggtgtccagccagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacttcgcccctttcttcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaagtcggcggcaactacaattacctgtaccggctgttccggaagtccaatctgaagcccttcgagcgggacatctccaccgagatctatcaggccggcaacaagccttgtaacggcgtggccggcttcaactgctacttcccactgcggtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtga

SEQ ID NO: 37 - protein, 1265 aa, synthetic construct
  source 1..1265: note = Amino Acid Sequence of BA.2
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 38 - DNA, 3621 bp, synthetic construct
  source 1..3621: mol_type = other DNA; note = Nucleotide Sequence of BA.2
atgttcgtgtttctggtgctgctgcctctggtgtccagccagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacttcgcccctttcttcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaagtcggcggcaactacaattacctgtaccggctgttccggaagtccaatctgaagcccttcgagcgggacatctccaccgagatctatcaggccggcaacaagccttgtaacggcgtggccggcttcaactgctacttcccactgcggtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 39 - protein, 1207 aa, synthetic construct
  source 1..1207: note = Amino Acid Sequence of BA.2
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 40 - DNA, 3582 bp, synthetic construct
  source 1..3582: mol_type = other DNA; note = Nucleotide Sequence of BA.2
cagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacttcgcccctttcttcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaagtcggcggcaactacaattacctgtaccggctgttccggaagtccaatctgaagcccttcgagcgggacatctccaccgagatctatcaggccggcaacaagccttgtaacggcgtggccggcttcaactgctacttcccactgcggtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 41 - protein, 1194 aa, synthetic construct
  source 1..1194: note = Amino Acid Sequence of BA.2
QCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 42 - DNA, 3798 bp, synthetic construct
  source 1..3798: mol_type = other DNA; note = Nucleotide Sequence of BA.2 S371 S373 S375
atgttcgtgtttctggtgctgctgcctctggtgtccagccagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaactccgccagcttcagcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaagtcggcggcaactacaattacctgtaccggctgttccggaagtccaatctgaagcccttcgagcgggacatctccaccgagatctatcaggccggcaacaagccttgtaacggcgtggccggcttcaactgctacttcccactgcggtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtga

SEQ ID NO: 43 - protein, 1265 aa, synthetic construct
  source 1..1265: note = Amino Acid Sequence of BA.2 S371 S373 S375
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNSASFSAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 44 - DNA, 3621 bp, synthetic construct
  source 1..3621: mol_type = other DNA; note = Nucleotide Sequence of BA.2 S371 S373 S375
atgttcgtgtttctggtgctgctgcctctggtgtccagccagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaactccgccagcttcagcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaagtcggcggcaactacaattacctgtaccggctgttccggaagtccaatctgaagcccttcgagcgggacatctccaccgagatctatcaggccggcaacaagccttgtaacggcgtggccggcttcaactgctacttcccactgcggtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 45 - protein, 1207 aa, synthetic construct
  source 1..1207: note = Amino Acid Sequence of BA.2 S371 S373 S375
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNSASFSAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 46 - DNA, 3582 bp, synthetic construct
  source 1..3582: mol_type = other DNA; note = Nucleotide Sequence of BA.2 S371 S373 S375
cagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaactccgccagcttcagcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaagtcggcggcaactacaattacctgtaccggctgttccggaagtccaatctgaagcccttcgagcgggacatctccaccgagatctatcaggccggcaacaagccttgtaacggcgtggccggcttcaactgctacttcccactgcggtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 47 - protein, 1194 aa, synthetic construct
  source 1..1194: note = Amino Acid Sequence of BA.2 S371 S373 S375
QCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNSASFSAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 48 - DNA, 3798 bp, synthetic construct
  source 1..3798: mol_type = other DNA; note = Nucleotide Sequence of BA.2.12.1
atgttcgtgtttctggtgctgctgcctctggtgtccagccagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacttcgcccctttcttcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaagtcggcggcaactacaattaccagtaccggctgttccggaagtccaatctgaagcccttcgagcgggacatctccaccgagatctatcaggccggcaacaagccttgtaacggcgtggccggcttcaactgctacttcccactgcggtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacctggtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtga

SEQ ID NO: 49 - protein, 1265 aa, synthetic construct
  source 1..1265: note = Amino Acid Sequence of BA.2.12.1
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYQYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENLVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 50 - DNA, 3621 bp, synthetic construct
  source 1..3621: mol_type = other DNA; note = Nucleotide Sequence of BA.2.12.1
atgttcgtgtttctggtgctgctgcctctggtgtccagccagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacttcgcccctttcttcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaagtcggcggcaactacaattaccagtaccggctgttccggaagtccaatctgaagcccttcgagcgggacatctccaccgagatctatcaggccggcaacaagccttgtaacggcgtggccggcttcaactgctacttcccactgcggtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacctggtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 51 - protein, 1207 aa, synthetic construct
  source 1..1207: note = Amino Acid Sequence of BA.2.12.1
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYQYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENLVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 52 - DNA, 3582 bp, synthetic construct
  source 1..3582: mol_type = other DNA; note = Nucleotide Sequence of BA.2.12.1
cagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacttcgcccctttcttcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaagtcggcggcaactacaattaccagtaccggctgttccggaagtccaatctgaagcccttcgagcgggacatctccaccgagatctatcaggccggcaacaagccttgtaacggcgtggccggcttcaactgctacttcccactgcggtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacctggtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 53 - protein, 1194 aa, synthetic construct
  source 1..1194: note = Amino Acid Sequence of BA.2.12.1
QCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYQYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENLVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 54 - DNA, 3798 bp, synthetic construct
  source 1..3798: mol_type = other DNA; note = Nucleotide Sequence of BA.2.12.1 S371 S373 S375
atgttcgtgtttctggtgctgctgcctctggtgtccagccagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaactccgccagcttcagcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaagtcggcggcaactacaattaccagtaccggctgttccggaagtccaatctgaagcccttcgagcgggacatctccaccgagatctatcaggccggcaacaagccttgtaacggcgtggccggcttcaactgctacttcccactgcggtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacctggtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtga

SEQ ID NO: 55 - protein, 1265 aa, synthetic construct
  source 1..1265: note = Amino Acid Sequence of BA.2.12.1 S371 S373 S375
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNSASFSAFKCYGVSPTKLNDLCFTNVYADSFVIRGNEVSQIAPGQTGNIADYNYKLPDDFTGCVIAWNSNKLDSKVGGNYNYQYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENLVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 56 - DNA, 3621 bp, synthetic construct
  source 1..3621: mol_type = other DNA; note = Nucleotide Sequence of BA.2.12.1 S371 S373 S375
atgttcgtgtttctggtgctgctgcctctggtgtccagccagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaactccgccagcttcagcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaagtcggcggcaactacaattaccagtaccggctgttccggaagtccaatctgaagcccttcgagcgggacatctccaccgagatctatcaggccggcaacaagccttgtaacggcgtggccggcttcaactgctacttcccactgcggtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacctggtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 57 - protein, 1207 aa, synthetic construct
  source 1..1207: note = Amino Acid Sequence of BA.2.12.1 S371 S373 S375
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNSASFSAFKCYGVSPTKLNDLCFTNVYADSFVIRGNEVSQIAPGQTGNIADYNYKLPDDFTGCVIAWNSNKLDSKVGGNYNYQYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENLVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 58 - DNA, 3582 bp, synthetic construct
  source 1..3582: mol_type = other DNA; note = Nucleotide Sequence of BA.2.12.1 S371 S373 S375
cagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaactccgccagcttcagcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaagtcggcggcaactacaattaccagtaccggctgttccggaagtccaatctgaagcccttcgagcgggacatctccaccgagatctatcaggccggcaacaagccttgtaacggcgtggccggcttcaactgctacttcccactgcggtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacctggtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 59 - protein, 1194 aa, synthetic construct
  source 1..1194: note = Amino Acid Sequence of BA.2.12.1 S371 S373 S375
QCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNSASFSAFKCYGVSPTKLNDLCFTNVYADSFVIRGNEVSQIAPGQTGNIADYNYKLPDDFTGCVIAWNSNKLDSKVGGNYNYQYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENLVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 60 - DNA, 3792 bp, synthetic construct
  source 1..3792: mol_type = other DNA; note = Nucleotide Sequence of BA.4/5
atgttcgtgtttctggtgctgctgcctctggtgtccagccagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacttcgcccctttcttcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaagtcggcggcaactacaattaccggtaccggctgttccggaagtccaatctgaagcccttcgagcgggacatctccaccgagatctatcaggccggcaacaagccttgtaacggcgtggccggcgtgaactgctacttcccactgcagtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtga

SEQ ID NO: 61 - protein, 1263 aa, synthetic construct
  source 1..1263: note = Amino Acid Sequence of BA.4/5
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 62 - DNA, 3615 bp, synthetic construct
  source 1..3615: mol_type = other DNA; note = Nucleotide Sequence of BA.4/5
atgttcgtgtttctggtgctgctgcctctggtgtccagccagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacttcgcccctttcttcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaagtcggcggcaactacaattaccggtaccggctgttccggaagtccaatctgaagcccttcgagcgggacatctccaccgagatctatcaggccggcaacaagccttgtaacggcgtggccggcgtgaactgctacttcccactgcagtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 63 - protein, 1205 aa, synthetic construct
  source 1..1205: note = Amino Acid Sequence of BA.4/5
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 64 - DNA, 3576 bp, synthetic construct
  source 1..3576: mol_type = other DNA; note = Nucleotide Sequence of BA.4/5
cagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacttcgcccctttcttcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaagtcggcggcaactacaattaccggtaccggctgttccggaagtccaatctgaagcccttcgagcgggacatctccaccgagatctatcaggccggcaacaagccttgtaacggcgtggccggcgtgaactgctacttcccactgcagtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 65 - protein, 1192 aa, synthetic construct
  source 1..1192: note = Amino Acid Sequence of BA.4/5
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 66 - DNA, 3792 bp, synthetic construct
  source 1..3792: mol_type = other DNA; note = Nucleotide Sequence of BA.4/5 S371 S373 S375
atgttcgtgtttctggtgctgctgcctctggtgtccagccagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaactccgccagcttcagcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaagtcggcggcaactacaattaccggtaccggctgttccggaagtccaatctgaagcccttcgagcgggacatctccaccgagatctatcaggccggcaacaagccttgtaacggcgtggccggcgtgaactgctacttcccactgcagtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtga

SEQ ID NO: 67 - protein, 1263 aa, synthetic construct
  source 1..1263: note = Amino Acid Sequence of BA.4/5 S371 S373 S375
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNSASFSAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 68 - DNA, 3615 bp, synthetic construct
  source 1..3615: mol_type = other DNA; note = Nucleotide Sequence of BA.4/5 S371 S373 S375
atgttcgtgtttctggtgctgctgcctctggtgtccagccagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaactccgccagcttcagcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaagtcggcggcaactacaattaccggtaccggctgttccggaagtccaatctgaagcccttcgagcgggacatctccaccgagatctatcaggccggcaacaagccttgtaacggcgtggccggcgtgaactgctacttcccactgcagtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 69 - protein, 1205 aa, synthetic construct
  source 1..1205: note = Amino Acid Sequence of BA.4/5 S371 S373 S375
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNSASFSAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 70 - DNA, 3576 bp, synthetic construct
  source 1..3576: mol_type = other DNA; note = Nucleotide Sequence of BA.4/5 S371 S373 S375
cagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaactccgccagcttcagcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaagtcggcggcaactacaattaccggtaccggctgttccggaagtccaatctgaagcccttcgagcgggacatctccaccgagatctatcaggccggcaacaagccttgtaacggcgtggccggcgtgaactgctacttcccactgcagtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 71 - protein, 1192 aa, synthetic construct
  source 1..1192: note = Amino Acid Sequence of BA.4/5 S371 S373 S375
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNSASFSAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 72 - protein, 6 aa, synthetic construct
  source 1..6: note = His tag
HHHHHH

SEQ ID NO: 73 - protein, 5 aa, synthetic construct
  source 1..5: note = Exemplary linker
  REPEAT 1..5: note = GGGGS can be repeated n times, where n is 1, 2, 3, 4, 5 or more
GGGGS

SEQ ID NO: 74 - DNA, 3795 bp, synthetic construct
  source 1..3795: mol_type = other DNA; note = Nucleotide Sequence of BA.5
atgttcgtgtttctggtgctgctgcctctggtgtccagccagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacttcgcccctttcttcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaagtcagcggcaactacaattaccggtaccggctgttccggaagtccaatctgaagcccttcgagcgggacatctccaccgagatctatcaggccggcaacaagccttgtaacggcgtggccggcgtgaactgctacttcccactgcagtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtgataa

SEQ ID NO: 75 - protein, 1263 aa, synthetic construct
  source 1..1263: note = Amino Acid Sequence of BA.5
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 76 - DNA, 3615 bp, synthetic construct
  source 1..3615: mol_type = other DNA; note = Nucleotide Sequence of BA.5
atgttcgtgtttctggtgctgctgcctctggtgtccagccagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacttcgcccctttcttcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaagtcagcggcaactacaattaccggtaccggctgttccggaagtccaatctgaagcccttcgagcgggacatctccaccgagatctatcaggccggcaacaagccttgtaacggcgtggccggcgtgaactgctacttcccactgcagtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 77 - protein, 1205 aa, synthetic construct
  source 1..1205: note = Amino Acid Sequence of BA.5
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 78 - DNA, 3576 bp, synthetic construct
  source 1..3576: mol_type = other DNA; note = Nucleotide Sequence of BA.5
cagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaacttcgcccctttcttcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaagtcagcggcaactacaattaccggtaccggctgttccggaagtccaatctgaagcccttcgagcgggacatctccaccgagatctatcaggccggcaacaagccttgtaacggcgtggccggcgtgaactgctacttcccactgcagtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 79 - protein, 1192 aa, synthetic construct
  source 1..1192: note = Amino Acid Sequence of BA.5
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 80 - DNA, 3792 bp, synthetic construct
  source 1..3792: mol_type = other DNA; note = Nucleotide Sequence of BA.5 S371 S373 S375 L452
atgttcgtgtttctggtgctgctgcctctggtgtccagccagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaactccgccagcttcagcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaagtcagcggcaactacaattacctgtaccggctgttccggaagtccaatctgaagcccttcgagcgggacatctccaccgagatctatcaggccggcaacaagccttgtaacggcgtggccggcgtgaactgctacttcccactgcagtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtga

SEQ ID NO: 81 - protein, 1263 aa, synthetic construct
  source 1..1263: note = Amino Acid Sequence of BA.5 S371 S373 S375 L452
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNSASFSAFKCYGVSPTKLNDLCFTNVYADSFVIRGNEVSQIAPGQTGNIADYNYKLPDDFTGCVIAWNSNKLDSKVSGNYNYLYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 82 - DNA, 3615 bp, synthetic construct
  source 1..3615: mol_type = other DNA; note = Nucleotide Sequence of BA.5 S371 S373 S375 L452
atgttcgtgtttctggtgctgctgcctctggtgtccagccagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaactccgccagcttcagcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaagtcagcggcaactacaattacctgtaccggctgttccggaagtccaatctgaagcccttcgagcgggacatctccaccgagatctatcaggccggcaacaagccttgtaacggcgtggccggcgtgaactgctacttcccactgcagtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 83 - protein, 1205 aa, synthetic construct
  source 1..1205: note = Amino Acid Sequence of BA.5 S371 S373 S375 L452
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNSASFSAFKCYGVSPTKLNDLCFTNVYADSFVIRGNEVSQIAPGQTGNIADYNYKLPDDFTGCVIAWNSNKLDSKVSGNYNYLYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 84 - DNA, 3576 bp, synthetic construct
  source 1..3576: mol_type = other DNA; note = Nucleotide Sequence of BA.5 S371 S373 S375 L452
cagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaactccgccagcttcagcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaagtcagcggcaactacaattacctgtaccggctgttccggaagtccaatctgaagcccttcgagcgggacatctccaccgagatctatcaggccggcaacaagccttgtaacggcgtggccggcgtgaactgctacttcccactgcagtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 85 - protein, 1192 aa, synthetic construct
  source 1..1192: note = Amino Acid Sequence of BA.5 S371 S373 S375 L452
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNSASFSAFKCYGVSPTKLNDLCFTNVYADSFVIRGNEVSQIAPGQTGNIADYNYKLPDDFTGCVIAWNSNKLDSKVSGNYNYLYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 86 - DNA, 3795 bp, synthetic construct
  source 1..3795: mol_type = other DNA; note = Nucleotide Sequence of BQ1.1
atgttcgtgtttctggtgctgctgcctctggtgtccagccagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccaccttcgcctctgtgtacgcctggaaccggaagcggatcagcaattgcgtggccgactactccgtgctgtacaacttcgcccctttcttcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaccgtcggcggcaactacaattaccggtaccggctgttccggaagtccaagctgaagcccttcgagcgggacatctccaccgagatctatcaggccggcaacaagccttgtaacggcgtggccggcgtgaactgctacttcccactgcagtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtgataa

SEQ ID NO: 87 - protein, 1263 aa, synthetic construct
  source 1..1263: note = Amino Acid Sequence of BQ.1.1
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 88 - DNA, 3615 bp, synthetic construct
  source 1..3615: mol_type = other DNA; note = Nucleotide Sequence of BQ1.1
atgttcgtgtttctggtgctgctgcctctggtgtccagccagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccaccttcgcctctgtgtacgcctggaaccggaagcggatcagcaattgcgtggccgactactccgtgctgtacaacttcgcccctttcttcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaccgtcggcggcaactacaattaccggtaccggctgttccggaagtccaagctgaagcccttcgagcgggacatctccaccgagatctatcaggccggcaacaagccttgtaacggcgtggccggcgtgaactgctacttcccactgcagtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 89 - protein, 1205 aa, synthetic construct
  source 1..1205: note = Amino Acid Sequence of BQ.1.1
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 90 - DNA, 3576 bp, synthetic construct
  source 1..3576: mol_type = other DNA; note = Nucleotide Sequence of BQ1.1
cagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatctccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcggccgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccaccttcgcctctgtgtacgcctggaaccggaagcggatcagcaattgcgtggccgactactccgtgctgtacaacttcgcccctttcttcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaccgtcggcggcaactacaattaccggtaccggctgttccggaagtccaagctgaagcccttcgagcgggacatctccaccgagatctatcaggccggcaacaagccttgtaacggcgtggccggcgtgaactgctacttcccactgcagtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 91 - protein, 1192 aa, synthetic construct
  source 1..1192: note = Amino Acid Sequence of BQ.1.1
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 92 - DNA, 3798 bp, synthetic construct
  source 1..3798: mol_type = other DNA; note = Nucleotide Sequence of XBB.1.5
atgttcgtgtttctggtgctgctgcctctggtgtccagccagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgctctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctatcagaagaacaacaagagctggatggaaagcgagttccgggtgtacagcagcgccaacaactgcaccttcgagtacgtgtcccagcctttcctgatggacctggaaggcaaggagggcaacttcaagaacctgcgcgagttcgtgttcaagaacatcgacggctacttcaagatctacagcaagcacacccctatcaacctcgagcgggatctgcctcagggcttctctgctctggaacccctggtggatctgcccatcggcatcaacatcacccggtttcagacactgctggccctgcacagaagctacctgacacctgtcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttccacgaggtgttcaatgccaccaccttcgcctctgtgtacgcctggaaccggaagcggatcagcaattgcgtggccgactactccgtgatctacaacttcgcccctttcttcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaacccagcggcaactacaattacctgtaccggctgttccggaagtccaagctgaagcccttcgagcgggacatctccaccgagatctatcaggccggcaacaagccttgtaacggcgtggccggccctaactgctactctccactgcagtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtgataa

SEQ ID NO: 93 - protein, 1264 aa, synthetic construct
  source 1..1264: note = Amino Acid Sequence of XBB.1.5
MFVFLVLLPLVSSQCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 94 - DNA, 3618 bp, synthetic construct
  source 1..3618: mol_type = other DNA; note = Nucleotide Sequence of XBB.1.5
atgttcgtgtttctggtgctgctgcctctggtgtccagccagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgctctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctatcagaagaacaacaagagctggatggaaagcgagttccgggtgtacagcagcgccaacaactgcaccttcgagtacgtgtcccagcctttcctgatggacctggaaggcaaggagggcaacttcaagaacctgcgcgagttcgtgttcaagaacatcgacggctacttcaagatctacagcaagcacacccctatcaacctcgagcgggatctgcctcagggcttctctgctctggaacccctggtggatctgcccatcggcatcaacatcacccggtttcagacactgctggccctgcacagaagctacctgacacctgtcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttccacgaggtgttcaatgccaccaccttcgcctctgtgtacgcctggaaccggaagcggatcagcaattgcgtggccgactactccgtgatctacaacttcgcccctttcttcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaacccagcggcaactacaattacctgtaccggctgttccggaagtccaagctgaagcccttcgagcgggacatctccaccgagatctatcaggccggcaacaagccttgtaacggcgtggccggccctaactgctactctccactgcagtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 95 - protein, 1206 aa, synthetic construct
  source 1..1206: note = Amino Acid Sequence of XBB.1.5
MFVFLVLLPLVSSQCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 96 - DNA, 3579 bp, synthetic construct
  source 1..3579: mol_type = other DNA; note = Nucleotide Sequence of XBB.1.5
cagtgtgtgaacctgatcacaagaacccagagctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgctctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctatcagaagaacaacaagagctggatggaaagcgagttccgggtgtacagcagcgccaacaactgcaccttcgagtacgtgtcccagcctttcctgatggacctggaaggcaaggagggcaacttcaagaacctgcgcgagttcgtgttcaagaacatcgacggctacttcaagatctacagcaagcacacccctatcaacctcgagcgggatctgcctcagggcttctctgctctggaacccctggtggatctgcccatcggcatcaacatcacccggtttcagacactgctggccctgcacagaagctacctgacacctgtcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttccacgaggtgttcaatgccaccaccttcgcctctgtgtacgcctggaaccggaagcggatcagcaattgcgtggccgactactccgtgatctacaacttcgcccctttcttcgccttcaagtgctacggcgtgtcccctaccaagctgaacgacctgtgcttcacaaacgtgtacgccgacagcttcgtgatccggggaaacgaagtgagccagattgcccctggacagacaggcaacatcgccgactacaactacaagctgcccgacgacttcaccggctgtgtgattgcctggaacagcaacaagctggactccaaacccagcggcaactacaattacctgtaccggctgttccggaagtccaagctgaagcccttcgagcgggacatctccaccgagatctatcaggccggcaacaagccttgtaacggcgtggccggccctaactgctactctccactgcagtcctacggctttcggcccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 97 - protein, 1193 aa, synthetic construct
  source 1..1193: note = Amino Acid Sequence of XBB.1.5
QCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 98 - DNA, 3798 bp, synthetic construct
  source 1..3798: mol_type = other DNA; note = Nucleotide Sequence of BA.1 S371 S375
atgttcgtgtttctggtgctgctgcctctggtgtccagccagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgtgatctccggcaccaatggcaccaagagattcgacaaccccgtgctgcccttcaacgacggggtgtactttgccagcatcgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcatcgtgcgggaacccgaagatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaactccgcccctttcagcaccttcaagtgctacggcgtgtcccctaccaagctgaacgacctgtgcttcacaaacgtgtacgccgacagcttcgtgatccggggagatgaagtgcggcagattgcccctggacagacaggcaacatcgccgactacaactacaagctgcccgacgacttcaccggctgtgtgattgcctggaacagcaacaagctggactccaaagtcagcggcaactacaattacctgtaccggctgttccggaagtccaatctgaagcccttcgagcgggacatctccaccgagatctatcaggccggcaacaagccttgtaacggcgtggccggcttcaactgctacttcccactgagatcctacagctttagacccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaagggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaagggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcttcagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtga

SEQ ID NO: 99 - protein, 1265 aa, synthetic construct
  source 1..1265: note = Amino Acid Sequence of BA.1 S371 S375
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNSAPFSTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFKGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 100 - DNA, 3621 bp, synthetic construct
  source 1..3621: mol_type = other DNA; note = Nucleotide Sequence of BA.1 S371 S375
atgttcgtgtttctggtgctgctgcctctggtgtccagccagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgtgatctccggcaccaatggcaccaagagattcgacaaccccgtgctgcccttcaacgacggggtgtactttgccagcatcgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcatcgtgcgggaacccgaagatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaactccgcccctttcagcaccttcaagtgctacggcgtgtcccctaccaagctgaacgacctgtgcttcacaaacgtgtacgccgacagcttcgtgatccggggagatgaagtgcggcagattgcccctggacagacaggcaacatcgccgactacaactacaagctgcccgacgacttcaccggctgtgtgattgcctggaacagcaacaagctggactccaaagtcagcggcaactacaattacctgtaccggctgttccggaagtccaatctgaagcccttcgagcgggacatctccaccgagatctatcaggccggcaacaagccttgtaacggcgtggccggcttcaactgctacttcccactgagatcctacagctttagacccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaagggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaagggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcttcagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 101 - protein, 1207 aa, synthetic construct
  source 1..1207: note = Amino Acid Sequence of BA.1 S371 S375
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNSAPFSTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFKGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 102 - DNA, 3582 bp, synthetic construct
  source 1..3582: mol_type = other DNA; note = Nucleotide Sequence of BA.1 S371 S375
cagtgtgtgaacctgaccacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgtgatctccggcaccaatggcaccaagagattcgacaaccccgtgctgcccttcaacgacggggtgtactttgccagcatcgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcatcgtgcgggaacccgaagatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcgacgaggtgttcaatgccaccagattcgcctctgtgtacgcctggaaccggaagcggatcagcaattgcgtggccgactactccgtgctgtacaactccgcccctttcagcaccttcaagtgctacggcgtgtcccctaccaagctgaacgacctgtgcttcacaaacgtgtacgccgacagcttcgtgatccggggagatgaagtgcggcagattgcccctggacagacaggcaacatcgccgactacaactacaagctgcccgacgacttcaccggctgtgtgattgcctggaacagcaacaagctggactccaaagtcagcggcaactacaattacctgtaccggctgttccggaagtccaatctgaagcccttcgagcgggacatctccaccgagatctatcaggccggcaacaagccttgtaacggcgtggccggcttcaactgctacttcccactgagatcctacagctttagacccacatacggcgtgggccaccagccctacagagtggtggtgctgagcttcgaactgctgcatgcccctgccacagtgtgcggccctaagaagagcaccaatctcgtgaagaacaaatgcgtgaacttcaacttcaacggcctgaagggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagtacgtgaacaatagctacgagtgcgacatccccatcggcgctggcatctgtgccagctaccagacacagacaaagagccacgcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaagagagccctgacagggatcgccgtggaacaggacaagaacacccaagaggtgttcgcccaagtgaagcagatctacaagacccctcctatcaagtacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaagggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcaggacgtggtcaaccacaatgcccaggcactgaacaccctggtcaagcagctgtcctccaagttcggcgccatcagctctgtgctgaacgatatcttcagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 103 - protein, 1194 aa, synthetic construct
  source 1..1194: note = Amino Acid Sequence of BA.1 S371 S375
QCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNSAPFSTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSPIEDLLFNKVTLADAGFIKQYGDCLGDIAARDLICAQKFKGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 104 - protein, 1213 aa, Synthetic construct
  source 1..1213: note = Amino Acid Sequence of Wuhan strain
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 105 - protein, 1200 aa, Synthetic construct
  source 1..1200: note = Amino Acid Sequence of Wuhan strain
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

(19) United States
(12) Patent Application Publication
(10) Pub. No.: US 2025/0228929 A1
(43) Pub. Date: Jul. 17, 2025

(54) RECOMBINANT NEWCASTLE DISEASE VIRUSES AND IMMUNOGENIC COMPOSITIONS FOR USE IN IMMUNIZING AGAINST SARS-COV-2 OMICRON VARIANT

(71) Applicant: ICAHN SCHOOL OF MEDICINE AT MOUNT SINAI, New York, NY (US)

(72) Inventors: Peter PALESE, New York, NY (US); Adolfo GARCIA-SASTRE, New York, NY (US); Florian KRAMMER, New York, NY (US); Weina SUN, New York, NY (US)

(21) Appl. No.: 18/853,695

(22) PCT Filed: Mar. 31, 2023
(86) PCT No.: PCT/US2023/065225

§ 371 Date: Oct. 2, 2024

Related U.S. Application Data
(60) Provisional application No. 63/326,877, filed on Apr. 3, 2022.
(60) Provisional application No. 63/346,262, filed on May 26, 2022.
(60) Provisional application No. 63/346,260, filed on May 26, 2022.

(51) Int. Cl. A61K 39/215; A61K 9/00; A61K 39/00; A61P 31/14; C07K 14/005; C12N 15/86

(52) U.S. Cl. CPC A61K 39/215; A61K 9/0043; A61P 31/14; C07K 14/005; C12N 15/86; A61K 2039/5256; A61K 2039/543; A61K 2039/55; C07K 2319/03; C07K 2319/71; C12N 2760/18122; C12N 2760/18134; C12N 2760/18143; C12N 2770/20022; C12N 2770/20034

(57) ABSTRACT

Described herein are recombinant Newcastle disease viruses (“NDVs”) comprising a packaged genome, wherein the packaged genome comprises a transgene comprising a nucleotide sequence encoding a protein comprising a SARS-CoV-2 Omicron variant spike protein or portion thereof, or a derivative thereof. Also described herein are recombinant NDVs comprising a packaged genome, wherein the packaged genome comprises a transgene encoding a chimeric F protein, wherein the chimeric F protein comprises a SARS-CoV-2 Omicron variant spike protein ectodomain or a derivative thereof, and NDV F protein transmembrane and cytoplasmic domains. Further, described herein are immunogenic compositions comprising a recombinant NDV(s). The recombinant NDVs and immunogenic compositions are useful for the immunizing against SARS-CoV-2 as well as the prevention of COVID-19.

### CROSS-REFERENCE TO RELATED APPLICATIONS

This application claims the benefit of U.S. Provisional Patent Application No. 63/346,262, filed May 26, 2022, U.S. Provisional Patent Application No. 63/346,260, filed May 26, 2022, and U.S. Provisional Patent Application No. 63/326,877, filed Apr. 3, 2022, the disclosure of each of which is incorporated by reference herein in its entirety.

### STATEMENT REGARDING FEDERALLY SPONSORED RESEARCH

This invention was made with government support under grant HHSN272201400008C awarded by the National Institutes of Health. The government has certain rights in the invention.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “06923-398-228_SEQLISTING.xml”, was created on Mar. 30, 2023 and is 380,403 bytes in size.

### 1. INTRODUCTION

In one aspect, described herein are recombinant Newcastle disease virus (“NDV”) comprising a packaged genome, wherein the packaged genome comprises a transgene encoding a protein comprising a spike protein of an Omicron variant of a severe acute respiratory syndrome coronavirus 2 (“SARS-CoV-2”) or a portion thereof (e.g., ectodomain or receptor binding domain of SARS-CoV-2 spike protein). In a specific embodiment, described herein are recombinant NDV comprising a packaged genome, wherein the packaged genome comprises a transgene comprising a codon-optimized nucleic acid sequence encoding a protein comprising a spike protein of an Omicron variant of a SARS-CoV-2 or portion thereof (e.g., ectodomain or receptor binding domain of SARS-CoV-2 spike protein). In a specific embodiment, described herein are recombinant NDV comprising a packaged genome, wherein the packaged genome comprises a transgene encoding a chimeric F protein, wherein the chimeric F protein comprises a spike protein ectodomain of an Omicron variant of a SARS-CoV-2 and NDV F protein transmembrane and cytoplasmic domains. In a specific embodiment, described herein are recombinant NDV comprising a packaged genome, wherein the packaged genome comprises a transgene encoding a chimeric F protein, wherein the chimeric F protein comprises a derivative of a spike protein ectodomain of an Omicron variant of a SARS-CoV-2 and NDV F protein transmembrane and cytoplasmic domains. Also described herein are compositions comprising such recombinant NDV(s) and the use of such recombinant NDV(s) as well as compositions to induce an immune response to SARS-CoV-2 an Omicron variant of spike protein, and in immunoassays to detect the presence of antibody that binds to SARS-CoV-2 spike protein. Further, provided herein are immunogenic compositions comprising recombinant NDVs and the use of such immunogenic compositions to immunize against SARS-CoV-2 as well as prevent COVID-19.

### 2. BACKGROUND

Severe acute respiratory syndrome coronavirus 2 (SARS-CoV-2) is the causative agent of the current coronavirus disease 2019 (COVID-19). Since the beginning of the pandemic, the emergence of new variants of concern (VOC) has threatened the protection conferred by vaccination using the original strain (Carreno et al., 2021. Evidence for retained spike-binding and neutralizing activity against emerging SARS-CoV-2 variants in serum of COVID-19 mRNA vaccine recipients. EBioMedicine 73:103626). In December 2020, the Alpha variant (B.1.1.7) and Beta variant (B.1.351) were declared VOC and spread over the world, followed by the Gamma strain (P.1) that was declared VOC in January 2021. Both Beta and Gamma variants exhibited notable resistance to neutralizing antibodies raised against the original strain in humans (Carreno et al., 2021. Evidence for retained spike-binding and neutralizing activity against emerging SARS-CoV-2 variants in serum of COVID-19 mRNA vaccine recipients. EBioMedicine 73:103626; Garcia-Beltran et al., 2021. Multiple SARS-CoV-2 variants escape neutralization by vaccine-induced humoral immunity. Cell 184:2372-2383 e9). In May 2021, a huge epidemic in India gave rise to a new VOC: the Delta variant (B.1.617.2). This new VOC harbored different mutations in the spike from other variants that also significantly reduced its sensitivity to neutralizing antibodies, and increased transmissibility quickly replacing the previous variants worldwide (Carreno et al., 2021. Evidence for retained spike-binding and neutralizing activity against emerging SARS-CoV-2 variants in serum of COVID-19 mRNA vaccine recipients. EBioMedicine 73:103626; Planas et al., 2021. Reduced sensitivity of SARS-CoV-2 variant Delta to antibody neutralization. Nature 596:276-280). In November 2021, a new VOC named Omicron appeared in South Africa. Since that moment, Omicron has taken over worldwide replacing the Delta variant (Holder J. 2022. Tracking Coranavirus Vaccination Around the World, on The New York Times. https://www.nytimes.com/interactive/2021/world/covid-vaccinations-tracker.html. Accessed 5 Feb. 2022). Compared to the previous VOC, Omicron presents the highest number of mutations in the spike protein and has shown the highest drop-in neutralization activity (Carreño et al., 2021. Activity of convalescent and vaccine serum against SARS-CoV-2 Omicron. Nature doi: 10.1038/d41586-021-03846-z; Hannah Ritchie et. al., 2020. Coronavirus Pandemic (COVID-19), on Our World Data. https://ourworldindata.org/covid-vaccinations. Accessed 05-Febraury-2022). Currently, the Omicron sub-linage BA.2, also known as the “stealth” Omicron seems to show even more immune evasion and transmissibility (Mahase E. 2022. Omicron sub-lineage BA.2 may have “substantial growth advantage,” UKHSA reports. BMJ 376:0263; Li et al., 2021. Omicron and S-gene target failure cases in the highest COVID-19 case rate region in Canada-December 2021. J Med Virol doi: 10.1002/jmv.27562; ECDC/WHO. 2021. Methods for the detection and characterisation of SARS-CoV-2 variants-first update. 20 Dec. 2021. Stockholm/Copenhagen).

Despite of the unprecedentedly rapid development of COVID-19 vaccines, only a 63.1% of the global population are fully vaccinated (Hannah Ritchie et. al., 2020. Coronavirus Pandemic (COVID-19), on Our World Data. https://ourworldindata.org/covid-vaccinations. Accessed 05-Febraury-2022). Hence, there is still a need for COVID-19 vaccines that can be produced locally in low- and middle-income countries (LMICs), where the vaccination rates are the lowest worldwide (id.).

### 3. SUMMARY

In one aspect, described herein are nucleotide sequences comprising severe acute respiratory syndrome coronavirus 2 (“SARS-CoV-2”) Omicron spike protein or a portion thereof (e.g., ectodomain or receptor binding domain of SARS-CoV-2 Omicron spike protein), or a derivative thereof. In a specific embodiment, encoding a chimeric F protein, wherein the chimeric F protein comprises an SARS-CoV-2 Omicron spike protein ectodomain or a derivative thereof and NDV F protein transmembrane and cytoplasmic domains.

In some embodiments, provided herein are nucleic acid sequences comprising a nucleotide sequence set forth in Table 3, infra. In some embodiments, provided herein is a nucleic acid sequence comprising the nucleotide sequence of SEQ ID NO:6, 10, 14, 30, 36, 42, 48, 54, 60, 66, 74, 80, 86, 92, or 98. In some embodiments, provided herein is a nucleic acid sequence comprising the nucleotide sequence of SEQ ID NO:6, 10, 14, 30, 36, 42, 48, 54, 60, 66, 74, 80, 86, 92, or 98 without the nucleotide sequence encoding the signal peptide. In some embodiments, provided herein is a nucleic acid sequence comprising the nucleotide sequence of SEQ ID NO:30, 42, 54, 66, 80, 86, 92, or 98. In some embodiments, provided herein is a nucleic acid sequence comprising the nucleotide sequence of SEQ ID NO:30, 42, 54, 66, 80, 86, 92, or 98 without the nucleotide sequence encoding the signal peptide. In some embodiments, provided herein is a nucleic acid sequence comprising the nucleotide sequence of SEQ ID NO:7, 11, or 15. In some embodiments, provided herein is a nucleic acid sequence comprising the nucleotide sequence of SEQ ID NO:36, 48, or 60, without the nucleotide sequence encoding the signal peptide. In some embodiments, provided herein is a nucleic acid sequence comprising the nucleotide sequence of SEQ ID NO:30, 42, 54, 66, 80, 92, or 98. In some embodiments, provided herein is a nucleic acid sequence comprising the nucleotide sequence of SEQ ID NO:30, 42, 54, 66, 80, 92, or 98 without the nucleotide sequence encoding the signal peptide. In some embodiments, provided herein is a nucleic acid sequence comprising the nucleotide sequence of an ectodomain set forth in Table 3. In some embodiments, provided herein is a nucleic acid sequence comprising the nucleotide sequence of an ectodomain set forth in Table 3, and the nucleotide sequence encoding NDV F protein transmembrane and cytoplasmic domains. In some embodiments, provided herein is a nucleic acid sequence comprising the nucleotide sequence of SEQ ID NO: 18, 20, 22, 34, 40, 46, 52, 58, 64, 70, 78, 84, 90, 96, or 102, and the nucleotide sequence encoding the transmembrane and cytoplasmic domains of NDV F protein. In some embodiments, provided herein is a nucleic acid sequence comprising the nucleotide sequence of SEQ ID NO:34, 46, 58, 84, 90, 96, or 102, and the nucleotide sequence encoding the transmembrane and cytoplasmic domains of NDV F protein. In some embodiments, provided herein is a nucleic acid sequence comprising the nucleotide sequence of SEQ ID NO:32, 34, 44, 46, 56, 58, 68, or 70, and the nucleotide sequence encoding the transmembrane and cytoplasmic domains of NDV F protein. In some embodiments, provided herein is a nucleic acid sequence comprising the nucleotide sequence of SEQ ID NO:32, 44, 56, 82, 88, 94, or 102, and the nucleotide sequence encoding the transmembrane and cytoplasmic domains of NDV F protein.

In some embodiments, provided herein are nucleic acid sequences encoding an amino acid sequence set forth in Table 3, infra. In some embodiments, provided herein is a nucleotide sequence encoding the amino acid sequence of SEQ ID NO:8, 12, 16, 31, 37, 43, 49, 55, 61, 67, 75, 81, 87, 93, or 99. In some embodiments, provided herein is a nucleotide sequence encoding the amino acid sequence of SEQ ID NO:8, 12, 16, 31, 37, 43, 49, 55, 61, 67, 75, 81, 87, 93, or 99, without the signal peptide. In some embodiments, provided herein is a nucleotide sequence encoding the amino acid sequence of SEQ ID NO: 31, 43, 55, 81, 87, 93, or 99. In some embodiments, provided herein is a nucleotide sequence encoding the amino acid sequence of SEQ ID NO: 31, 43, 55, 81, 87, 93, or 99, without the signal peptide. In some embodiments, provided herein is a nucleotide sequence encoding the amino acid sequence of SEQ ID NO:9, 13, or 17. In some embodiments, provided herein is a nucleotide sequence encoding the amino acid sequence of SEQ ID NO:37, 49, or 61. In some embodiments, provided herein is a nucleotide sequence encoding the amino acid sequence of SEQ ID NO:37, 49, or 61, without the signal peptide. In some embodiments, provided herein is a nucleotide sequence encoding the amino acid sequence of SEQ ID NO:31, 43, 55, 61, 67, 81, or 87. In some embodiments, provided herein is a nucleotide sequence encoding the amino acid sequence of SEQ ID NO:31, 43, 55, 61, 67, 81, or 87, without the signal peptide. In some embodiments, provided herein is a nucleic acid sequence encoding the amino acid sequence of an ectodomain set forth in Table 3. In some embodiments, provided herein is a nucleic acid sequence encoding the amino acid sequence of an ectodomain set forth in Table 3, and the nucleotide sequence encoding NDV F protein transmembrane and cytoplasmic domains. In some embodiments, provided herein is a nucleic acid sequence comprising the nucleotide sequence encoding the amino acid sequence of SEQ ID NO: 19, 21, 23, 39, 41, 51, 53, 63, or 65, and the nucleotide sequence encoding the transmembrane and cytoplasmic domains of NDV F protein. In some embodiments, provided herein is a nucleic acid sequence comprising the nucleotide sequence encoding the amino acid sequence of SEQ ID NO: 19, 21, 23, 35, 41, 47, 53, 59, 65, 71, 79, 85, 91, 97, or 103, and the nucleotide sequence encoding the transmembrane and cytoplasmic domains of NDV F protein. In some embodiments, provided herein is a nucleic acid sequence comprising the nucleotide sequence encoding the amino acid sequence of SEQ ID NO: 35, 47, 59, 85, 91, 97, or 103, and the nucleotide sequence encoding the transmembrane and cytoplasmic domains of NDV F protein. In some embodiments, provided herein is a nucleic acid sequence comprising the nucleotide sequence encoding the amino acid sequence of SEQ ID NO: 33, 39, 45, 51, 57, 63, 69, 77, 83, 89, 95, or 101, and the nucleotide sequence encoding the transmembrane and cytoplasmic domains of NDV F protein. In some embodiments, provided herein is a nucleic acid sequence comprising the nucleotide sequence encoding the amino acid sequence of SEQ ID NO: 33, 45, 57, 83, 89, 95, or 101, and the nucleotide sequence encoding the transmembrane and cytoplasmic domains of NDV F protein. In some embodiments, provided herein a nucleic acid sequence comprising the nucleotide sequence encoding the amino acid sequence of SEQ ID NO:33, 35, 45, 47, 57, 59, 69, or 71, and the nucleotide sequence encoding the transmembrane and cytoplasmic domains of NDV F protein.

In some embodiments, provided herein is a recombinant protein comprising a SARS-CoV-2 Omicron spike protein ectodomain or portion thereof described herein. The SARS-CoV-2 Omicron spike protein ectodomain or portion thereof may be any one described herein in the context of a transgene. In some embodiments, provided herein is a recombinant protein comprising a derivative of a SARS-CoV-2 Omicron spike protein ectodomain described herein. The derivative of the SARS-CoV-2 Omicron spike protein ectodomain may be any one described herein in the context of a transgene. In some embodiments, provided herein is a recombinant protein comprising a derivative of a SARS-CoV-2 Omicron spike protein ectodomain, wherein the derivative comprises the ectodomain of the amino acid sequence of SEQ ID NO:104 without the signal peptide and with amino acid modifications, wherein the amino acid modifications comprise: (1) an amino acid substitution at amino acid residues corresponding to amino acid residues 682 to 685 (RRAR) of SEQ ID NO:104 to a single alanine; (2) amino acid substitutions at amino acid residues corresponding to the following amino acid residues of SEQ ID NO: 104: F817P, A892P, A899P, A942P, K986P, and V987P; and (3) two or more amino acid modifications to the amino acid sequence of the ectodomain of SEQ ID NO: 104 to amino acid residues found at the corresponding amino acid positions in the Omicron spike protein ectodomain, wherein the two or more amino acid modifications comprise two or more amino acid modifications at amino acid positions corresponding to the indicated amino acid positions of SEQ ID NO: 104: N440K, S477N, Y505H, N679K, N764K, D796Y, Q954H, and/or N969K. In some embodiments, the two or more amino acid modifications comprise the following amino acid modifications at amino acid positions corresponding to the indicated amino acid positions of SEQ ID NO: 104: N440K, S477N, Y505H, N679K, N764K, D796Y, Q954H, and N969K. In some embodiments, the two or more amino acid modifications does not include amino acid modifications at amino acid positions corresponding to amino acid positions of 371 and 375 in SEQ ID NO:104. In some embodiments, the two or more amino acid modifications does not include amino acid modifications at amino acid positions corresponding to amino acid positions of 371, 373, and 375 in SEQ ID NO:104. In some embodiments, the two or more amino acid modifications does not comprise amino acid modifications does not include amino acid modification at the amino acid position corresponding to amino acid position of 452 in SEQ ID NO:104. In some embodiments, the two or more amino acid modifications does not include amino acid modifications at amino acid positions corresponding to amino acid positions of 371, 375, and 452 in SEQ ID NO: 104. In some embodiments, the two or more amino acid modifications does not include amino acid modifications at amino acid positions corresponding to amino acid positions of 371, 373, 375, and 452 in SEQ ID NO: 104. In some embodiments, the two or more amino acid modifications further comprises the following amino acid modification at the amino acid position corresponding to the indicated amino acid positions of SEQ ID NO: 104: G339D or G339H.

In some embodiments, the two or more amino acid modifications comprise two or more (e.g., 3, 4, 5, 6, 7, 8, 9, 10, 11, 12, 13, 14, 15, 16, or more) or all of the amino acid modifications of a construct in Table 5. In some embodiments, the two or more amino acid modifications comprise two or more (e.g., 3, 4, 5, 6, 7, 8, 9, 10, 11, 12, 13, 14, 15, 16, or more) or all of the amino acid modifications of a construct in Table 6. In some embodiments, the two or more amino acid modifications comprise two or more (e.g., 3, 4, 5, 6, 7, 8, 9, 10, 11, 12, 13, 14, 15, 16, or more) or all of the amino acid modifications of a construct in Table 7. In some embodiments, the two or more amino acid modifications comprise two or more (e.g., 3, 4, 5, 6, 7, 8, 9, 10, 11, 12, 13, 14, 15, 16, or more) or all of the amino acid modifications of a construct in Table 8. In some embodiments, the two or more amino acid modifications comprise two or more (e.g., 3, 4, 5, 6, 7, 8, 9, 10, 11, 12, 13, 14, 15, 16, or more) or all of the amino acid modifications of a construct in Table 9. In some embodiments, the two or more amino acid modifications comprise two or more (e.g., 3, 4, 5, 6, 7, 8, 9, 10, 11, 12, 13, 14, 15, 16, or more) or all of the amino acid modifications of a construct in Table 10. In some embodiments, the two or more amino acid modifications comprise two or more (e.g., 3, 4, 5, 6, 7, 8, 9, 10, 11, 12, 13, 14, 15, 16, or more) or all of the amino acid modifications of a construct in Table 11.

In some embodiments, the two or more amino acid modifications comprise two or more (e.g., 3, 4, 5, 6, 7, 8, 9, 10, 11, 12, 13, 14, 15, 16, or more) or all of the following amino acid modifications at amino acid positions corresponding to the indicated amino acid positions of SEQ ID NO: 104: A67V, HV69-70 deletion, T95I, G142D, VYY143-145 deletion, N211 deletion, L212I, ins214EPE, G339D, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F. In some embodiments, the two or more amino acid modifications further comprise one or more (e.g., 1, 2, 3, 4, 5, or more) of the following amino acid modifications at amino acid positions corresponding to the indicated amino acid positions of SEQ ID NO: 104: T19I, del24-26 (LPP), T376A, D405N, R408S, and/or Q498R. In some embodiments, the two or more amino acid modifications further comprise one or more of the following amino acid modifications at amino acid positions corresponding to the indicated amino acid positions of SEQ ID NO: 104: T19I, del24-26 (LPP), T376A, D405N, R408S, and Q498R. In some embodiments, the two or more amino acid modifications further comprise the following amino acid modification at the amino acid position corresponding to the indicated amino acid position of SEQ ID NO:104: V213G or V213E. In some embodiments, the two or more amino acid modifications comprise two or more (e.g., 3, 4, 5, 6, 7, 8, 9, 10, 11, 12, 13, 14, 15, 16, or more) or all of the following amino acid modifications at amino acid positions corresponding to the indicated amino acid positions of SEQ ID NO: 104: T19I, del24-26 (LPP), A27S, G142D, V213G, G339D, T376A, D405N, R408S, K417N, N440K, S477N, T478K, E484A, Q493R, Q498R, N501Y, Y505H, D614G, H655Y, N679K, P681H, N764K, D796Y, Q954H, and N969K. In some embodiments, the two or more amino acid modifications comprise two or more (e.g., 3, 4, 5, 6, 7, 8, 9, 10, 11, 12, 13, 14, 15, 16, or more) or all of the following amino acid modifications at the amino acid positions corresponding to the indicated amino acid positions of SEQ ID NO: 104: T19I, del24-26 (LPP), A27S, del69-70, G142D, V213G, G339D, R346T, T376A, D405N, R408S, K417N, N440K, K444T, L452R, N460K, S477N, T478K, E484A, F486V, Q498R, N501Y, Y505H, D614G, H655Y, N679K, P681H, N764K, D796Y, Q954H, and N969K. In some embodiments, the two or more amino acid modifications comprise the two or more (e.g., 3, 4, 5, 6, 7, 8, 9, 10, 11, 12, 13, 14, 15, 16, or more) or all of following amino acid modifications at the amino acid positions corresponding to the indicated amino acid positions of SEQ ID NO:104: T19I, del24-26 (LPP), A27S, V83A, G142D, del144, H146Q, Q183E, V213E, G252V, G339H, R346T, L368I, T376A, D405N, R408S, K417N, N440K, V445P, G446S, N460K, S477N, T478K, E484A, F486P, F490S, Q498R, N501Y, Y505H, D614G, H655Y, N679K, P681H, N764K, D796Y, Q954H, and N969K. In some embodiments, the two or more amino acid modifications does not include amino acid modification at the amino acid position corresponding to amino acid position of 452 in SEQ ID NO:104. In some embodiments, the two or more amino acid modifications comprise two or more (e.g., 3, 4, 5, 6, 7, 8, 9, 10, 11, 12, 13, 14, 15, 16, or more) or all of the following amino acid modifications at the amino acid positions corresponding to the indicated amino acid positions of SEQ ID NO: 104: T19I, del24-26 (LPP), A27S, del69-70(HV), G142D, V213G, G339D, T376A, D405N, R408S, K417N, N440K, S477N, L452R, T478K, E484A, F486V, Q498R, N501Y, Y505H, D614G, H655Y, N679K, P681H, N764K, D796Y, Q954H, and N969K.

In some embodiments, provided herein is a recombinant protein comprising a derivative of a SARS-CoV-2 Omicron spike protein ectodomain, wherein the derivative comprises the ectodomain of the amino acid sequence of SEQ ID NO: 104 without the signal peptide and with amino acid modifications, wherein the amino acid modifications comprise: (1) an amino acid substitution at amino acid residues corresponding to amino acid residues 682 to 685 (RRAR) of SEQ ID NO:104 to a single alanine; (2) amino acid substitutions at amino acid residues corresponding to the following amino acid residues of SEQ ID NO:104: F817P, A892P, A899P, A942P, K986P, and V987P; and (3) two or more (e.g., 3, 4, 5, 6, 7, 8, 9, 10, 11, 12, 13, 14, 15, 16, 17, 18, or more) amino acid modifications to the amino acid sequence of the ectodomain of SEQ ID NO: 104 to amino acid residues found at the corresponding amino acid positions in the Omicron spike protein ectodomain. In some embodiments, the two or more (e.g., 3, 4, 5, 6, 7, 8, 9, 10, 11, 12, 13, 14, 15, 16, 17, 18, or more) amino acid modifications do not include amino acid modifications at the amino acid positions corresponding to the amino acid positions 371 and 375 of SEQ ID NO:104. In some embodiments, the two or more (e.g., 3, 4, 5, 6, 7, 8, 9, 10, 11, 12, 13, 14, 15, 16, 17, 18, or more) amino acid modifications do not include amino acid modifications at the amino acid positions corresponding to the amino acid positions 371, 373, and 375 of SEQ ID NO: 104.

In some embodiments, the two or more (e.g., 3, 4, 5, 6, 7, 8, 9, 10, 11, 12, 13, 14, 15, 16, 17, 18, or more) amino acid modifications two or more (e.g., 3, 4, 5, 6, 7, 8, 9, 10, 11, 12, 13, 14, 15, 16, 17, 18, or more) or all of the following amino acid modifications at amino acid positions corresponding to the indicated amino acid positions of SEQ ID NO: 104: A67V, HV69-70 deletion, T95I, G142D, VYY143-145 deletion, N211 deletion, L212I, ins214EPE, G339D, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F. In some embodiments, the two or more (e.g., 3, 4, 5, 6, 7, 8, 9, 10, 11, 12, 13, 14, 15, 16, 17, 18, or more) amino acid modifications two or more (e.g., 3, 4, 5, 6, 7, 8, 9, 10, 11, 12, 13, 14, 15, 16, 17, 18, or more) or all of the following amino acid modifications at amino acid positions corresponding to the indicated amino acid positions of SEQ ID NO: 104: T19I, del24-26 (LPP), A27S, G142D, V213G, G339D, T376A, D405N, R408S, K417N, N440K, S477N, T478K, E484A, Q493R, Q498R, N501Y, Y505H, D614G, H655Y, N679K, P681H, N764K, D796Y, Q954H, and N969K. In some embodiments, the two or more (e.g., 3, 4, 5, 6, 7, 8, 9, 10, 11, 12, 13, 14, 15, 16, 17, 18, or more) amino acid modifications two or more (e.g., 3, 4, 5, 6, 7, 8, 9, 10, 11, 12, 13, 14, 15, 16, 17, 18, or more) or all of the following amino acid modifications at amino acid positions corresponding to the indicated amino acid positions of SEQ ID NO: 104: T19I, del24-26 (LPP), A27S, del69-70, G142D, V213G, G339D, R346T, T376A, D405N, R408S, K417N, N440K, K444T, L452R, N460K, S477N, T478K, E484A, F486V, Q498R, N501Y, Y505H, D614G, H655Y, N679K, P681H, N764K, D796Y, Q954H, and N969K. In some embodiments, the two or more (e.g., 3, 4, 5, 6, 7, 8, 9, 10, 11, 12, 13, 14, 15, 16, 17, 18, or more) amino acid modifications two or more (e.g., 3, 4, 5, 6, 7, 8, 9, 10, 11, 12, 13, 14, 15, 16, 17, 18, or more) or all of the following amino acid modifications at amino acid positions corresponding to the indicated amino acid positions of SEQ ID NO: 104: T19I, del24-26 (LPP), A27S, V83A, G142D, del144, H146Q, Q183E, V213E, G252V, G339H, R346T, L368I, T376A, D405N, R408S, K417N, N440K, V445P, G446S, N460K, S477N, T478K, E484A, F486P, F490S, Q498R, N501Y, Y505H, D614G, H655Y, N679K, P681H, N764K, D796Y, Q954H, and N969K. In some embodiments, the two or more (e.g., 3, 4, 5, 6, 7, 8, 9, 10, 11, 12, 13, 14, 15, 16, 17, 18, or more) amino acid modifications two or more (e.g., 3, 4, 5, 6, 7, 8, 9, 10, 11, 12, 13, 14, 15, 16, 17, 18, or more) or all of the following amino acid modifications at amino acid positions corresponding to the indicated amino acid positions of SEQ ID NO: 104: T19I, del24-26 (LPP), A27S, del69-70, G142D, V213G, G339D, R346T, S371F, S373P, S375F, T376A, D405N, R408S, K417N, N440K, K444T, L452R, N460K, S477N, T478K, E484A, F486V, Q498R, N501Y, Y505H, D614G, H655Y, N679K, P681H, N764K, D796Y, Q954H, and N969K. In some embodiments, the two or more (e.g., 3, 4, 5, 6, 7, 8, 9, 10, 11, 12, 13, 14, 15, 16, 17, 18, or more) amino acid modifications two or more (e.g., 3, 4, 5, 6, 7, 8, 9, 10, 11, 12, 13, 14, 15, 16, 17, 18, or more) or all of the following amino acid modifications at the amino acid positions corresponding to the indicated amino acid positions of SEQ ID NO: 104: T19I, del24-26 (LPP), A27S, V83A, G142D, del144, H146Q, Q183E, V213E, G252V, G339H, R346T, L368I, S371F, S373P, S375F, T376A, D405N, R408S, K417N, N440K, V445P, G446S, N460K, S477N, T478K, E484A, F486P, F490S, Q498R, N501Y, Y505H, D614G, H655Y, N679K, P681H, N764K, D796Y, Q954H, and N969K. In some embodiments, the two or more (e.g., 3, 4, 5, 6, 7, 8, 9, 10, 11, 12, 13, 14, 15, 16, 17, 18, or more) amino acid modifications two or more (e.g., 3, 4, 5, 6, 7, 8, 9, 10, 11, 12, 13, 14, 15, 16, 17, 18, or more) or all of the following amino acid modifications at amino acid positions corresponding to the indicated amino acid positions of SEQ ID NO: 104: T19I, del24-26 (LPP), A27S, del69-70 (HV), G142D, V213G, G339D, T376A, D405N, R408S, K417N, N440K, S477N, L452R, T478K, E484A, F486V, Q498R, N501Y, Y505H, D614G, H655Y, N679K, P681H, N764K, D796Y, Q954H, and N969K.

In some embodiments, the two or more amino acid modifications are 18 or more amino acid modifications. In some embodiments, the two or more amino acid modifications are 19, 20, 21, 22, 23, 24, 25, 26, 27, or 28 amino acid modifications.

In some embodiments, a recombinant protein described herein further comprises a trimerization domain (e.g., a T4 foldon trimerization domain). In some embodiments, a recombinant protein described herein further comprises NDV F protein transmembrane and cytoplasmic domains.

In some embodiments, the derivative of the ectodomain comprises the amino acid sequence of SEQ ID NO: 103, 35, 85, 47, 59, 91, 97, 19, 21, 23, 41, 53, 65, 71, or 79. In some embodiments, the derivative of the ectodomain comprises the amino acid sequence of SEQ ID NO: 103, 35, 85, 47, 59, 91, 97.

In some embodiments, provided herein is a recombinant protein comprising a derivative of the ectodomain of a SARS-CoV-2 variant, wherein the ectodomain comprises an amino acid sequence that is at least 90% identical to the amino acid sequence of SEQ ID NO: 103, 35, 85, 47, 59, 91, 97, 19, 21, 23, 41, 53, 65, 71, 79, 33, 39, 45, 51, 57, 63, 69, 77, 83, 89, 95 or 101. In some embodiments, provided herein is a recombinant protein comprising a derivative of the ectodomain of a SARS-CoV-2 variant, wherein the ectodomain comprises an amino acid sequence that is at least 90% identical to the amino acid sequence of SEQ ID NO: 103, 35, 85, 47, 59, 91, 97. In some embodiments, the derivative of the ectodomain comprises: (1) alanine at amino acid residues corresponding to amino acid residues 682 to 685 (RRAR) of the amino acid sequence of SEQ ID NO:104; (2) proline at amino acid residues corresponding to the following amino acid residues of the amino acid sequence of SEQ ID NO: 104: F817, A892, A899, A942, K986, and V987; and (3) two or more of the following amino acid residues at amino acid positions corresponding to the indicated amino acid positions of the amino acid sequence of SEQ ID NO: 104: 440K, 477N, 505H, 679K, 764K, 796Y, 954H, and/or 969K. In some embodiments, the ectodomain comprises the amino acid sequence of SEQ ID NO: 103, 35, 85, 47, 59, 91, 97, 19, 21, 23, 41, 53, 65, 71, 79, 33, 39, 45, 51, 57, 63, 69, 77, 83, 89, 95 or 101.

In some embodiments, the protein further comprises a signal peptide. In some embodiments, the signal peptide comprises the amino acid sequence of SEQ ID NO:29.

In some embodiments, the protein further comprises the transmembrane and cytoplasmic domains of NDV F protein. In some embodiments, the protein further comprises a linker and the transmembrane and cytoplasmic domains of NDV F protein. In some embodiments, the transmembrane and cytoplasmic domains of NDV F protein comprises the amino acid sequence of SEQ ID NO: 5.

In some embodiments, provided herein is a polynucleotide comprising a nucleotide sequence encoding a protein described herein (e.g., a recombinant protein described herein). In some embodiments, the nucleotide sequence comprises the nucleotide sequence of SEQ ID NO: 30, 42, 54, 66, 80, 86, 92, 98, 6, 7, 10, 11, 14, 15, 36, 48, 60 or 74. In some embodiments, the nucleotide sequence comprises the nucleotide sequence SEQ ID NO: 30, 42, 54, 66, 80, 86, 92, or 98. In some embodiments, the nucleotide sequence comprises the nucleotide sequence SEQ ID NO: 32, 82, 100, 88, or 94. In some embodiments, the nucleotide sequence encodes the amino acid sequence of SEQ ID NO: 35, 85, 91, 97, 103, 59, 19, 21, 23, 41, 47, 53, 65, 71, 79, 33, 83, 89, 95, 101, 57, 39, 45, 51, 63, 69, or 77. In some embodiments, the nucleotide sequence encodes the amino acid sequence of SEQ ID NO: 35, 85, 91, 97, 103, or 59. In some embodiments, the nucleotide sequence encodes the amino acid sequence of SEQ ID NO: 33, 83, 89, 95, 101, or 57. In some embodiments, the nucleotide sequence comprises the nucleotide sequence of SEQ ID NO: 30, 42, 54, 66, 80, 86, 92, 98, 6, 7, 10, 11, 14, 15, 36, 48, 60 or 74, without the nucleotide sequence encoding the signal peptide. In some embodiments, the nucleotide sequence comprises the nucleotide sequence of SEQ ID NO: 30, 42, 54, 66, 80, 86, 92, 98, 6, 7, 10, 11, 14, 15, 36, 48, 60, or 74. In some embodiments, the nucleotide sequence comprises the nucleotide sequence of SEQ ID NO: 6, 11, or 15. In some embodiments, the nucleotide sequence encodes the amino acid sequence of SEQ ID NO: 31, 43, 55, 67, 81, 87, 93, 99, 8, 12, 16, 37, 49, 61, or 75, without the signal peptide. In some embodiments, the nucleotide sequence encodes the amino acid sequence of SEQ ID NO: 31, 43, 55, 67, 81, 87, 93, 99, 8, 12, 16, 37, 49, 61, or 75. In some embodiments, the nucleotide sequence encodes the amino acid sequence of SEQ ID NO: 9, 13, 17.

In some embodiments, provided herein is a vector comprising a polynucleotide described herein. In some embodiments, the vector is a plasmid or a viral vector.

In some embodiments, provided herein is a transgene comprising a nucleotide sequence encoding a chimeric F protein, wherein the chimeric F protein comprises the amino acid sequence of SEQ ID NO: 31, 43, 55, 67, 81, 87, 93, 99, 8, 12, 16, 37, 49, 61, or 75, without the signal peptide. In some embodiments, provided herein is a transgene comprising a nucleotide sequence encoding a chimeric F protein, wherein the chimeric F protein comprises the amino acid sequence of SEQ ID NO: 31, 43, 55, 67, 81, 87, 93, or 99, without the signal peptide. In some embodiments, provided herein is a transgene comprising a nucleotide sequence encoding a chimeric F protein, wherein the chimeric F protein comprises the amino acid sequence of the amino acid sequence of SEQ ID NO: 31, 43, 55, 67, 81, 87, 93, 99, 8, 12, 16, 37, 49, 61, or 75. In some embodiments, provided herein is a transgene comprising a nucleotide sequence encoding a chimeric F protein, wherein the chimeric F protein comprises the amino acid sequence of the amino acid sequence of SEQ ID NO: 31, 43, 55, 67, 81, 87, 93, or 99.

In some embodiments, provided herein is a transgene comprising a nucleotide sequence encoding a chimeric F protein, wherein the chimeric F protein comprises an amino acid sequence that is at least 80% (e.g., at least 85%, at least 90%, at least 91%, at least 92%, at least 93%, or at least 94%) identical to SEQ ID NO: 31, 43, 55, 67, 81, 87, 93, 99, 8, 12, 16, 37, 49, 61, or 75, without the signal peptide. In some embodiments, provided herein is a transgene comprising a nucleotide sequence encoding a chimeric F protein, wherein the chimeric F protein comprises an amino acid sequence that is at least 80% (e.g., at least 85%, at least 90%, at least 91%, at least 92%, at least 93%, or at least 94%) identical to SEQ ID NO: 31, 43, 55, 67, 81, 87, 93, or 99, without the signal peptide. In some embodiments, provided herein is a transgene comprising a nucleotide sequence encoding a chimeric F protein, wherein the chimeric F protein comprises an amino acid sequence that is at least 80% (e.g., at least 85%, at least 90%, at least 91%, at least 92%, at least 93%, or at least 94%) identical to SEQ ID NO: 31, 43, 55, 67, 81, 87, 93, 99, 8, 12, 16, 37, 49, 61, or 75. In some embodiments, provided herein is a transgene comprising a nucleotide sequence encoding a chimeric F protein, wherein the chimeric F protein comprises an amino acid sequence that is at least 80% (e.g., at least 85%, at least 90%, at least 91%, at least 92%, at least 93%, or at least 94%) identical to SEQ ID NO: 31, 43, 55, 67, 81, 87, 93, or 99.

In some embodiments, provided herein is a transgene comprising a nucleotide sequence encoding a chimeric F protein, wherein the chimeric F protein comprises an amino acid sequence that is at least 90% (e.g., at least 91%, at least 92%, at least 93%, or at least 94%) identical to SEQ ID NO: 31, 43, 55, 67, 81, 87, 93, 99, 8, 12, 16, 37, 49, 61, or 75, without the signal peptide. In some embodiments, provided herein is a transgene comprising a nucleotide sequence encoding a chimeric F protein, wherein the chimeric F protein comprises an amino acid sequence that is at least 90% (e.g., at least 91%, at least 92%, at least 93%, or at least 94%) identical to SEQ ID NO: 31, 43, 55, 67, 81, 87, 93, or 99, without the signal peptide. In some embodiments, provided herein is a transgene comprising a nucleotide sequence encoding a chimeric F protein, wherein the chimeric F protein comprises an amino acid sequence that is at least 90% (e.g., at least 91%, at least 92%, at least 93%, or at least 94%) identical to SEQ ID NO: 31, 43, 55, 67, 81, 87, 93, 99, 8, 12, 16, 37, 49, 61, or 75. In some embodiments, provided herein is a transgene comprising a nucleotide sequence encoding a chimeric F protein, wherein the chimeric F protein comprises an amino acid sequence that is at least (e.g., at least 91%, at least 92%, at least 93%, or at least 94%) identical to SEQ ID NO: 31, 43, 55, 67, 81, 87, 93, or 99.

In some embodiments, provided herein is a transgene comprising a nucleotide sequence encoding a chimeric F protein, wherein the chimeric F protein comprises an amino acid sequence that is at least 95%, at least 96%, at least 97%, at least 98%, or at least 99% identical to SEQ ID NO: 31, 43, 55, 67, 81, 87, 93, 99, 8, 12, 16, 37, 49, 61, or 75, without the signal peptide. In some embodiments, provided herein is a transgene comprising a nucleotide sequence encoding a chimeric F protein, wherein the chimeric F protein comprises an amino acid sequence that is at least 95%, at least 96%, at least 97%, at least 98%, or at least 99% identical to SEQ ID NO: 31, 43, 55, 67, 81, 87, 93, or 99, without the signal peptide. In some embodiments, provided herein is a transgene comprising a nucleotide sequence encoding a chimeric F protein, wherein the chimeric F protein comprises an amino acid sequence that is at least 95%, at least 96%, at least 97%, at least 98%, or at least 99% identical to SEQ ID NO: 31, 43, 55, 67, 81, 87, 93, 99, 8, 12, 16, 37, 49, 61, or 75. In some embodiments, provided herein is a transgene comprising a nucleotide sequence encoding a chimeric F protein, wherein the chimeric F protein comprises an amino acid sequence that is at least 95%, at least 96%, at least 97%, at least 98%, or at least 99% identical to SEQ ID NO: 31, 43, 55, 67, 81, 87, 93, or 99. In some embodiments, provided herein is a transgene comprising a nucleotide sequence encoding a chimeric F protein, wherein the chimeric F protein comprises the amino acid sequence of SEQ ID NO: 31, 43, 55, 67, 81, 87, 93, 99, 8, 12, 16, 37, 49, 61, or 75. In some embodiments, provided herein is a transgene comprising a nucleotide sequence encoding a chimeric F protein, wherein the chimeric F protein comprises the amino acid sequence of SEQ ID NO: 31, 43, 55, 67, 81, 87, 93, or 99.

In some embodiments, provided herein is a transgene comprising a nucleotide sequence encoding a chimeric F protein, wherein the chimeric F protein comprises the amino acid sequence of SEQ ID NO: 8, 12, 16, 37, 49, 61, or 75, or an amino acid sequence that is at least 90% identical to SEQ ID NO: 88, 12, 16, 37, 49, 61, or 75. In some embodiments, the chimeric F protein comprises an amino acid sequence that is at least 90% identical to SEQ ID NO: 8, 12, 16, 37, 49, 61, or 75. In some embodiments, the chimeric F protein comprises an amino acid sequence that is at least 95%, at least 98%, or at least 99% identical to SEQ ID NO: 8, 12, 16, 37, 49, 61, or 75. In some embodiments, the chimeric F protein comprises the amino acid sequence of SEQ ID NO: 8, 12, 16, 37, 49, 61, or 75.

In some embodiments, provided herein is a transgene comprising the nucleotide sequence of SEQ ID NO: 30, 42, 54, 66, 80, 86, 92, 98, 6, 7, 10, 11, 14, 15, 36, 48, 60 or 74, without the nucleotide sequence encoding the signal peptide. In some embodiments, provided herein is a transgene comprising the nucleotide sequence of SEQ ID NO: 30, 42, 54, 66, 80, 86, 92, or 98, without the nucleotide sequence encoding the signal peptide. In some embodiments, provided herein is a transgene comprising the nucleotide sequence of SEQ ID NO: 30, 42, 54, 66, 80, 86, 92, 98, 6, 7, 10, 11, 14, 15, 36, 48, 60 or 74. In some embodiments, provided herein is a transgene comprising the nucleotide sequence of SEQ ID NO: 30, 42, 54, 66, 80, 86, 92, or 98.

In some embodiments, provided herein is a transgene comprising a nucleotide sequence that is at least 80% (e.g., at least 85%, at least 90%, at least 91%, at least 92%, at least 93%, or at least 94%) identical to SEQ ID NO: 30, 42, 54, 66, 80, 86, 92, 98, 6, 7, 10, 11, 14, 15, 36, 48, 60 or 74, without the nucleotide sequence encoding the signal peptide. In some embodiments, provided herein is a transgene comprising a nucleotide sequence that is at least 80% (e.g., at least 85%, at least 90%, at least 91%, at least 92%, at least 93%, or at least 94%) identical to SEQ ID NO: 30, 42, 54, 66, 80, 86, 92, or 98, without the nucleotide sequence encoding the signal peptide. In some embodiments, provided herein is a transgene comprising a nucleotide sequence that is at least 80% (e.g., at least 85%, at least 90%, at least 91%, at least 92%, at least 93%, or at least 94%) identical to SEQ ID NO: 30, 42, 54, 66, 80, 86, 92, 98, 6, 7, 10, 11, 14, 15, 36, 48, 60 or 74. In some embodiments, provided herein is a transgene comprising a nucleotide sequence that is at least 80% (e.g., at least 85%, at least 90%, at least 91%, at least 92%, at least 93%, or at least 94%) identical to SEQ ID NO: 30, 42, 54, 66, 80, 86, 92, or 98.

In some embodiments, provided herein is a transgene comprising a nucleotide sequence that is at least 90% (e.g., at least 91%, at least 92%, at least 93%, or at least 94%) identical to SEQ ID NO: 30, 42, 54, 66, 80, 86, 92, 98, 6, 7, 10, 11, 14, 15, 36, 48, 60 or 74, without the nucleotide sequence encoding the signal peptide. In some embodiments, provided herein is a transgene comprising a nucleotide sequence that is at least 90% (e.g., at least 91%, at least 92%, at least 93%, or at least 94%) identical to SEQ ID NO: 30, 42, 54, 66, 80, 86, 92, or 98, without the nucleotide sequence encoding the signal peptide. In some embodiments, provided herein is a transgene comprising a nucleotide sequence that is at least 90% (e.g., at least 91%, at least 92%, at least 93%, or at least 94%) identical to SEQ ID NO: 30, 42, 54, 66, 80, 86, 92, 98, 6, 7, 10, 11, 14, 15, 36, 48, 60 or 74. In some embodiments, provided herein is a transgene comprising a nucleotide sequence that is at least 90% (e.g., at least 91%, at least 92%, at least 93%, or at least 94%) identical to SEQ ID NO: 30, 42, 54, 66, 80, 86, 92, or 98.

In some embodiments, provided herein is a transgene comprising a nucleotide sequence that is at least 95%, at least 96%, at least 97%, at least 98%, or at least 99% identical to SEQ ID NO: 30, 42, 54, 66, 80, 86, 92, 98, 6, 10, 14, 36, 48, 60, or 74, without the nucleotide sequence encoding the signal peptide. In some embodiments, provided herein is a transgene comprising a nucleotide sequence that is at least 95%, at least 96%, at least 97%, at least 98%, or at least 99% identical to SEQ ID NO: 30, 42, 54, 66, 80, 86, 92, or 98, without the nucleotide sequence encoding the signal peptide. In some embodiments, provided herein is a transgene comprising a nucleotide sequence that is at least 95%, at least 96%, at least 97%, at least 98%, or at least 99% identical to SEQ ID NO: 30, 42, 54, 66, 80, 86, 92, 98, 6, 10, 14, 36, 48, 60, or 74. In some embodiments, provided herein is a transgene comprising a nucleotide sequence that is at least 95%, at least 96%, at least 97%, at least 98%, or at least 99% identical to SEQ ID NO: 30, 42, 54, 66, 80, 86, 92, or 98.

In some embodiments, provided herein is a transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises the nucleotide sequence of SEQ ID NO: 34, 84, 102, 90, 96, 46, 58, 18, 20, 22, 40, 52, 64, 70, 78, 32, 38, 44, 50, 56, 62, 68, 76, 82, 88, 94, or 100. In some embodiments, provided herein is a transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises the nucleotide sequence of SEQ ID NO: 34, 84, 102, 90, 96, 46, 58, 18, 20, 22, 40, 52, 64, 70, or 78. In some embodiments, provided herein is a transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises the nucleotide sequence of SEQ ID NO: 34, 84, 102, 90, 96, 46, or 58. In some embodiments, the SARS-CoV-2 Omicron virus spike protein ectodomain is linked via a linker to the NDV F protein transmembrane and cytoplasmic domains. In some embodiments, the linker comprises the amino acid sequence of SEQ ID NO:24.

In some embodiments, provided herein is a transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises the nucleotide sequence of SEQ ID NO: 32, 38, 44, 50, 56, 62, 68, 76, 82, 88, 94, or 100. In some embodiments, provided herein is a transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises the nucleotide sequence of SEQ ID NO:32, 44, 82, 88, 94, 100, or 56. In some embodiments, the SARS-CoV-2 Omicron virus spike protein ectodomain is linked via a linker to the NDV F protein transmembrane and cytoplasmic domains. In some embodiments, the linker comprises the amino acid sequence of SEQ ID NO: 24.

In some embodiments, provided herein is a transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises a nucleotide sequence that is at least 80% (e.g., at least 85%, at least 90%, at least 91%, at least 92%, at least 93%, or at least 94%) identical to the nucleotide sequence of SEQ ID NO: 34, 84, 102, 90, 96, 46, 58, 18, 20, 22, 40, 52, 64, 70, 78, 32, 38, 44, 50, 56, 62, 68, 76, 82, 88, 94, or 100. In some embodiments, provided herein is a transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises a nucleotide sequence that is at least 80% (e.g., at least 85%, at least 90%, at least 91%, at least 92%, at least 93%, or at least 94%) identical to the nucleotide sequence of SEQ ID NO: 34, 84, 102, 90, 96, 46, 58, 18, 20, 22, 40, 52, 64, 70, or 78. In some embodiments, provided herein is a transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises a nucleotide sequence that is at least 80% (e.g., at least 85%, at least 90%, at least 91%, at least 92%, at least 93%, or at least 94%) identical to the nucleotide sequence of SEQ ID NO: 34, 84, 102, 90, 96, 46, or 58. In some embodiments, the SARS-CoV-2 Omicron virus spike protein ectodomain is linked via a linker to the NDV F protein transmembrane and cytoplasmic domains. In some embodiments, the linker comprises the amino acid sequence of SEQ ID NO:24.

In some embodiments, provided herein is a transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises a nucleotide sequence that is at least 80% (e.g., at least 85%, at least 90%, at least 91%, at least 92%, at least 93%, or at least 94%) identical to the nucleotide sequence of SEQ ID NO: 32, 38, 44, 50, 56, 62, 68, 76, 82, 88, 94, or 100. In some embodiments, provided herein is a transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises a nucleotide sequence that is at least 80% (e.g., at least 85%, at least 90%, at least 91%, at least 92%, at least 93%, or at least 94%) identical to the nucleotide sequence of SEQ ID NO: 32, 82, 88, 94, 100, 44, or 56. In some embodiments, the SARS-CoV-2 Omicron virus spike protein ectodomain is linked via a linker to the NDV F protein transmembrane and cytoplasmic domains. In some embodiments, the linker comprises the amino acid sequence of SEQ ID NO:24.

In some embodiments, provided herein is a transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises a nucleotide sequence that is at least 95%, at least 96%, at least 97%, at least 98%, or at least 99% identical to the nucleotide sequence of SEQ ID NO: 34, 84, 102, 90, 96, 46, 58, 18, 20, 22, 40, 52, 64, 70, or 78. In some embodiments, provided herein is a transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises a nucleotide sequence that is at least 95%, at least 96%, at least 97%, at least 98%, or at least 99% identical to the nucleotide sequence of SEQ ID NO: 34, 84, 102, 90, 96, 46, or 58. In some embodiments, the SARS-CoV-2 Omicron virus spike protein ectodomain is linked via a linker to the NDV F protein transmembrane and cytoplasmic domains. In some embodiments, the linker comprises the amino acid sequence of SEQ ID NO:24.

In some embodiments, provided herein is a transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises a nucleotide sequence that is at least 95%, at least 96%, at least 97%, at least 98%, or at least 99% identical to the nucleotide sequence of SEQ ID NO: 32, 38, 44, 50, 56, 62, 68, 76, 82, 88, 94, or 100. In some embodiments, provided herein is a transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises a nucleotide sequence that is at least 95%, at least 96%, at least 97%, at least 98%, or at least 99% identical to the nucleotide sequence of SEQ ID NO: 32, 82, 88, 94, 100, 44, or 56. In some embodiments, the SARS-CoV-2 Omicron virus spike protein ectodomain is linked via a linker to the NDV F protein transmembrane and cytoplasmic domains. In some embodiments, the linker comprises the amino acid sequence of SEQ ID NO:24.

In some embodiments, provided herein is a transgene comprising an RNA sequence corresponding to the negative sense of the cDNA sequence of SEQ ID NO: 30, 42, 54, 66, 80, 86, 92, 98, 6, 10, 14, 36, 48, 60, or 74, without the nucleotide sequence encoding the signal peptide. In some embodiments, provided herein is a transgene comprising an RNA sequence corresponding to the negative sense of the cDNA sequence of SEQ ID NO: 30, 42, 54, 60, 80, 86, 92, or 98, without the nucleotide sequence encoding the signal peptide. In some embodiments, provided herein is a transgene comprising an RNA sequence corresponding to the negative sense of the cDNA sequence of SEQ ID NO: 30, 42, 54, 66, 80, 86, 92, 98, 6, 10, 14, 36, 48, 60, or 74. In some embodiments, provided herein is a transgene comprising an RNA sequence corresponding to the negative sense of the cDNA sequence of SEQ ID NO: 30, 42, 54, 60, 80, 86, 92, or 98.

In some embodiments, provided herein is a transgene comprising an RNA sequence corresponding to the negative sense of the cDNA sequence of a nucleotide sequence that is at least 80% (e.g., at least 85%, at least 90%, at least 91%, at least 92%, at least 93%, or at least 94%) identical to the cDNA sequence of SEQ ID NO: 30, 42, 54, 66, 80, 86, 92, 98, 6, 10, 14, 36, 48, 60, or 74, without the nucleotide sequence encoding the signal peptide. In some embodiments, provided herein is a transgene comprising an RNA sequence corresponding to the negative sense of the cDNA sequence of a nucleotide sequence that is at least 80% (e.g., at least 85%, at least 90%, at least 91%, at least 92%, at least 93%, or at least 94%) identical to the cDNA sequence of SEQ ID NO: 30, 42, 54, 60, 80, 86, 92, or 98, without the nucleotide sequence encoding the signal peptide. In some embodiments, provided herein is a transgene comprising an RNA sequence corresponding to the negative sense of the cDNA sequence of a nucleotide sequence that is at least 80% (e.g., at least 85%, at least 90%, at least 91%, at least 92%, at least 93%, or at least 94%) identical to the cDNA sequence of SEQ ID NO: 30, 42, 54, 66, 80, 86, 92, 98, 6, 10, 14, 36, 48, 60, or 74. In some embodiments, provided herein is a transgene comprising an RNA sequence corresponding to the negative sense of the cDNA sequence of a nucleotide sequence that is at least 80% (e.g., at least 85%, at least 90%, at least 91%, at least 92%, at least 93%, or at least 94%) identical to the cDNA sequence of SEQ ID NO: 30, 42, 54, 60, 80, 86, 92, or 98.

In some embodiments, provided herein is a transgene comprising an RNA sequence corresponding to the negative sense of the cDNA sequence of a nucleotide sequence that is at least 95%, at least 96%, at least 97%, at least 98%, or at least 99% identical to the cDNA sequence of SEQ ID NO: 30, 42, 54, 66, 80, 86, 92, 98, 6, 10, 14, 36, 48, 60, or 74, without the nucleotide sequence encoding the signal peptide. In some embodiments, provided herein is a transgene comprising an RNA sequence corresponding to the negative sense of the cDNA sequence of a nucleotide sequence that is at least 95%, at least 96%, at least 97%, at least 98%, or at least 99% identical to the cDNA sequence of SEQ ID NO: 30, 42, 54, 60, 80, 86, 92, or 98, without the nucleotide sequence encoding the signal peptide. In some embodiments, provided herein is a transgene comprising an RNA sequence corresponding to the negative sense of the cDNA sequence of a nucleotide sequence that is at least 95%, at least 96%, at least 97%, at least 98%, or at least 99% identical to the cDNA sequence of SEQ ID NO: 30, 42, 54, 66, 80, 86, 92, 98, 6, 10, 14, 36, 48, 60, or 74. In some embodiments, provided herein is a transgene comprising an RNA sequence corresponding to the negative sense of the cDNA sequence of a nucleotide sequence that is at least 95%, at least 96%, at least 97%, at least 98%, or at least 99% identical to the cDNA sequence of SEQ ID NO: 30, 42, 54, 60, 80, 86, 92, or 98.

In some embodiments, provided herein is a transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises an RNA sequence corresponding to the negative sense of the cDNA sequence of SEQ ID NO: 34, 84, 102, 90, 96, 46, 58, 18, 20, 22, 40, 52, 64, 70, or 78. In some embodiments, provided herein is a transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises an RNA sequence corresponding to the negative sense of the cDNA sequence of SEQ ID NO: 34, 84, 102, 90, 96, 46, or 58. In some embodiments, provided herein is a transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises an RNA sequence corresponding to the negative sense of the cDNA sequence of SEQ ID NO: 32, 82, 100, 88, 94, 44, 56, 38, 50, 62, 68, or 76. In some embodiments, provided herein is a transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises an RNA sequence corresponding to the negative sense of the cDNA sequence of SEQ ID NO: 32, 82, 100, 88, 94, 44, or 56. In some embodiments, the SARS-CoV-2 Omicron virus spike protein ectodomain is linked via a linker to the NDV F protein transmembrane and cytoplasmic domains. In some embodiments, the linker comprises the amino acid sequence of SEQ ID NO:24.

In some embodiments, provided herein is a transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises an RNA sequence corresponding to the negative sense of the cDNA sequence of a nucleotide sequence that is at least 80% (e.g., at least 85%, at least 90%, at least 91%, at least 92%, at least 93%, or at least 94%) identical to the cDNA sequence of SEQ ID NO: 34, 84, 102, 90, 96, 46, 58, 18, 20, 22, 40, 52, 64, 70, or 78. In some embodiments, provided herein is a transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises an RNA sequence corresponding to the negative sense of the cDNA sequence of a nucleotide sequence that is at least 80% (e.g., at least 85%, at least 90%, at least 91%, at least 92%, at least 93%, or at least 94%) identical to the cDNA sequence of SEQ ID NO: 34, 84, 102, 90, 96, 46, or 58. In some embodiments, the SARS-CoV-2 Omicron virus spike protein ectodomain is linked via a linker to the NDV F protein transmembrane and cytoplasmic domains. In some embodiments, the linker comprises the amino acid sequence of SEQ ID NO: 24.

In some embodiments, provided herein is a transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises an RNA sequence corresponding to the negative sense of the cDNA sequence of a nucleotide sequence that is at least 95%, at least 96%, at least 97%, at least 98%, or at least 99% identical to the cDNA sequence of SEQ ID NO: 34, 84, 102, 90, 96, 46, 58, 18, 20, 22, 40, 52, 64, 70, or 78. In some embodiments, provided herein is a transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises an RNA sequence corresponding to the negative sense of the cDNA sequence of a nucleotide sequence that is at least 95%, at least 96%, at least 97%, at least 98%, or at least 99% identical to the cDNA sequence of SEQ ID NO: 34, 84, 102, 90, 96, 46, or 58. In some embodiments, the SARS-CoV-2 Omicron virus spike protein ectodomain is linked via a linker to the NDV F protein transmembrane and cytoplasmic domains. In some embodiments, the linker comprises the amino acid sequence of SEQ ID NO:24.

In some embodiments, provided herein is a transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises an RNA sequence corresponding to the negative sense of the cDNA sequence of a nucleotide sequence that is at least 95%, at least 96%, at least 97%, at least 98%, or at least 99% identical to the cDNA sequence of SEQ ID NO: 32, 82, 100, 88, 94, 44, 56, 38, 50, 62, 68, or 76. In some embodiments, provided herein is a transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises an RNA sequence corresponding to the negative sense of the cDNA sequence of a nucleotide sequence that is at least 95%, at least 96%, at least 97%, at least 98%, or at least 99% identical to the cDNA sequence of SEQ ID NO: 32, 82, 100, 88, 94, 44, or 56. In some embodiments, the SARS-CoV-2 Omicron virus spike protein ectodomain is linked via a linker to the NDV F protein transmembrane and cytoplasmic domains. In some embodiments, the linker comprises the amino acid sequence of SEQ ID NO:24.

In some embodiments, provided herein is a transgene comprising a nucleotide sequence encoding a chimeric F protein, wherein the chimeric F protein comprises a derivative of a SARS-CoV-2 Omicron virus spike protein ectodomain and NDV F protein transmembrane and cytoplasmic domains, and wherein the derivative of the SARS-CoV-2 Omicron virus spike protein ectodomain comprises the amino acid sequence of SEQ ID NO: 35, 85, 103, 91, 97, 47, 59, 19, 21, 23, 41, 53, 65, 71, or 79. In some embodiments, provided herein is a transgene comprising a nucleotide sequence encoding a chimeric F protein, wherein the chimeric F protein comprises a derivative of a SARS-CoV-2 Omicron virus spike protein ectodomain and NDV F protein transmembrane and cytoplasmic domains, and wherein the derivative of the SARS-CoV-2 Omicron virus spike protein ectodomain comprises the amino acid sequence of SEQ ID NO: 35, 85, 103, 91, 97, 47, or 59. In some embodiments, provided herein is a transgene comprising a nucleotide sequence encoding a chimeric F protein, wherein the chimeric F protein comprises a derivative of a SARS-CoV-2 Omicron virus spike protein ectodomain and NDV F protein transmembrane and cytoplasmic domains, and wherein the derivative of the SARS-CoV-2 Omicron virus spike protein ectodomain comprises an amino acid sequence at least 80% (e.g., at least 85%, at least 90%, at least 91%, at least 92%, at least 93%, or at least 94%) identical to the amino acid sequence of SEQ ID NO: 35, 85, 103, 91, 97, 47, 59, 19, 21, 23, 41, 53, 65, 71, or 79. In some embodiments, provided herein is a transgene comprising a nucleotide sequence encoding a chimeric F protein, wherein the chimeric F protein comprises a derivative of a SARS-CoV-2 Omicron virus spike protein ectodomain and NDV F protein transmembrane and cytoplasmic domains, and wherein the derivative of the SARS-CoV-2 Omicron virus spike protein ectodomain comprises an amino acid sequence at least 90% (e.g., at least 91%, at least 92%, at least 93%, or at least 94%) identical to the amino acid sequence of SEQ ID NO: 35, 85, 103, 91, 97, 47, 59, 19, 21, 23, 41, 53, 65, 71, or 79. In some embodiments, the derivative of the SARS-CoV-2 Omicron virus spike protein ectodomain comprises an amino acid sequence at least 90% (e.g., at least 85%, at least 90%, at least 91%, at least 92%, at least 93%, or at least 94%) identical to the amino acid sequence of SEQ ID NO: 35, 85, 103, 91, 97, 47, or 59. In some embodiments, the derivative of the SARS-CoV-2 Omicron virus spike protein ectodomain comprises an amino acid sequence at least 95%, at least 98%, or at least 99% identical to the amino acid sequence of SEQ ID NO: 35, 85, 103, 91, 97, 47, 59, 19, 21, 23, 41, 53, 65, 71, or 79. In some embodiments, the derivative of the SARS-CoV-2 Omicron virus spike protein ectodomain comprises an amino acid sequence at least 95%, at least 98%, or at least 99% identical to the amino acid sequence of SEQ ID NO: 35, 85, 103, 91, 97, 47, or 59. In some embodiments, the SARS-CoV-2 Omicron virus spike protein ectodomain is linked via a linker to the NDV F protein transmembrane and cytoplasmic domains. In some embodiments, the linker comprises the amino acid sequence of SEQ ID NO:24.

In some embodiments, provided herein is a transgene comprising a nucleotide sequence encoding a chimeric F protein, wherein the chimeric F protein comprises a derivative of a SARS-CoV-2 Omicron virus spike protein ectodomain and NDV F protein transmembrane and cytoplasmic domains, and wherein the derivative of the SARS-CoV-2 Omicron virus spike protein ectodomain comprises the amino acid sequence of SEQ ID NO: 33, 83, 101, 89, 95, 45, 57, 39, 51, 63, 69, or 77. In some embodiments, provided herein is a transgene comprising a nucleotide sequence encoding a chimeric F protein, wherein the chimeric F protein comprises a derivative of a SARS-CoV-2 Omicron virus spike protein ectodomain and NDV F protein transmembrane and cytoplasmic domains, and wherein the derivative of the SARS-CoV-2 Omicron virus spike protein ectodomain comprises the amino acid sequence of SEQ ID NO: 33, 83, 101, 89, 95, 45, or 57. In some embodiments, provided herein is a transgene comprising a nucleotide sequence encoding a chimeric F protein, wherein the chimeric F protein comprises a derivative of a SARS-CoV-2 Omicron virus spike protein ectodomain and NDV F protein transmembrane and cytoplasmic domains, and wherein the derivative of the SARS-CoV-2 Omicron virus spike protein ectodomain comprises amino acid sequence at least 80% (e.g., at least 85%, at least 90%, at least 91%, at least 92%, at least 93%, or at least 94%) identical to the amino acid sequence of SEQ ID NO: 33, 83, 101, 89, 95, 45, 57, 39, 51, 63, 69, or 77. In some embodiments, provided herein is a transgene comprising a nucleotide sequence encoding a chimeric F protein, wherein the chimeric F protein comprises a derivative of a SARS-CoV-2 Omicron virus spike protein ectodomain and NDV F protein transmembrane and cytoplasmic domains, and wherein the derivative of the SARS-CoV-2 Omicron virus spike protein ectodomain comprises amino acid sequence at least 90% (e.g., at least 91%, at least 92%, at least 93%, or at least 94%) identical to the amino acid sequence of SEQ ID NO: 33, 83, 101, 89, 95, 45, 57, 39, 51, 63, 69, or 77. In some embodiments, provided herein is a transgene comprising a nucleotide sequence encoding a chimeric F protein, wherein the chimeric F protein comprises a derivative of a SARS-CoV-2 Omicron virus spike protein ectodomain and NDV F protein transmembrane and cytoplasmic domains, and wherein the derivative of the SARS-CoV-2 Omicron virus spike protein ectodomain comprises amino acid sequence at least 90% (e.g., at least 91%, at least 92%, at least 93%, or at least 94%) identical to the amino acid sequence of SEQ ID NO: 33, 83, 101, 89, 95, 45, or 57. In some embodiments, the derivative of the SARS-CoV-2 Omicron virus spike protein ectodomain comprises an amino acid sequence at least 95%, at least 98%, or at least 99% identical to the amino acid sequence of SEQ ID NO: 33, 35, 45, 47, 57, 59, 69, 83, 85, 89, 91, 95, 97, 103, or 71. In some embodiments, the derivative of the SARS-CoV-2 Omicron virus spike protein ectodomain comprises an amino acid sequence at least 95%, at least 98%, or at least 99% identical to the amino acid sequence of SEQ ID NO: 33, 35, 45, 47, 57, or 59. In some embodiments, the SARS-CoV-2 Omicron virus spike protein ectodomain is linked via a linker to the NDV F protein transmembrane and cytoplasmic domains. In some embodiments, the linker comprises the amino acid sequence of SEQ ID NO:24.

In some embodiments, provided herein is a transgene comprising the nucleotide sequence of SEQ ID NO:6, 7, 10, 11, 14, 15, 36, 48, or 60, or a nucleotide sequence that is at least 80% identical to SEQ ID NO: 6, 7, 10, 11, 14, 15, 36, 48, or 60. In some embodiments, the transgene comprises a nucleotide sequence that is at least 80% identical to SEQ ID NO: 6, 7, 10, 11, 14, 15, 36, 48, or 60. In some embodiments, the transgene comprises a nucleotide sequence that is at least 85%, at least 90%, at least 95%, at least 98%, or at least 99% identical to SEQ ID NO: 6, 7, 10, 11, 14, 15, 36, 48, or 60. In some embodiments, the transgene comprises the nucleotide sequence of SEQ ID NO: 6, 7, 10, 11, 14, 15, 36, 48, or 60.

In some embodiments, provided herein is a transgene comprising the nucleotide sequence of SEQ ID NO:30, 42, 54, 80, 86, 92, 98, or 66, or a nucleotide sequence that is at least 80% identical to SEQ ID NO: 30, 42, 54, 80, 86, 92, 98, or 66. In some embodiments, the transgene comprises a nucleotide sequence that is at least 80% identical to SEQ ID NO: 30, 42, 54, 80, 86, 92, 98, or 66. In some embodiments, the transgene comprises a nucleotide sequence that is at least 85%, at least 90%, at least 95%, at least 98%, or at least 99% identical to SEQ ID NO: 30, 42, 54, 80, 86, 92, 98, or 66. In some embodiments, the transgene comprises the nucleotide sequence of SEQ ID NO: 30, 42, 54, or 66.

In some embodiments, provided herein is a transgene comprising the nucleotide sequence of SEQ ID NO:30, 42, 54, 80, 86, 92, 98, or 66, without the signal peptide, or a nucleotide sequence that is at least 80% identical to SEQ ID NO: 30, 42, 54, 80, 86, 92, 98, or 66, without the signal peptide. In some embodiments, the transgene comprises a nucleotide sequence that is at least 80% identical to SEQ ID NO: 30, 42, 54, 80, 86, 92, 98, or 66, without the signal peptide. In some embodiments, the transgene comprises a nucleotide sequence that is at least 85%, at least 90%, at least 95%, at least 98%, or at least 99% identical to SEQ ID NO: 30, 42, 54, 80, 86, 92, 98, or 66, without the signal peptide. In some embodiments, the transgene comprises the nucleotide sequence of SEQ ID NO: 30, 42, 54, 80, 86, 92, 98, or 66, without the signal peptide.

In some embodiments, provided herein is a transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises the nucleotide sequence of SEQ ID NO:18, 20, 22, 38, 40, 50, 52, 62, or 64, or a nucleotide sequence that is at least 80% identical to the nucleotide sequence of SEQ ID NO: 18, 20, 22, 38, 40, 50, 52, 62, or 64. In some embodiments, the nucleotide sequence encoding the derivative of the SARS-CoV-2 Omicron virus spike protein ectodomain comprises a nucleotide sequence that is at least 80% identical to the nucleotide sequence of SEQ ID NO: 18, 20, 22, 38, 40, 50, 52, 62, or 64. In some embodiments, the nucleotide sequence encoding the derivative of the SARS-CoV-2 Omicron virus spike protein ectodomain comprises a nucleotide sequence that is at least 85%, at least 90%, at least 95%, at least 98%, or at least 99% identical to the nucleotide sequence of SEQ ID NO:18, 20, 22, 38, 40, 50, 52, 62, or 64. In some embodiments, the nucleotide sequence encoding the derivative of the SARS-CoV-2 Omicron virus spike protein ectodomain comprises the nucleotide sequence of SEQ ID NO:18, 20, 22, 38, 40, 50, 52, 62, or 64. In some embodiments, the SARS-CoV-2 Omicron virus spike protein ectodomain is linked via a linker to the NDV F protein transmembrane and cytoplasmic domains. In some embodiments, the linker comprises the amino acid sequence of SEQ ID NO:24.

In some embodiments, provided herein is a transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises the nucleotide sequence of SEQ ID NO:32, 34, 82, 84, 88, 90, 94, 96, 100, 102, 44, 46, 56, 58, 68, or 70, or a nucleotide sequence that is at least 80% identical to the nucleotide sequence of SEQ ID NO: 32, 34, 82, 84, 88, 90, 94, 96, 100, 102, 44, 46, 56, 58, 68, or 70. In some embodiments, the nucleotide sequence encoding the derivative of the SARS-CoV-2 Omicron virus spike protein ectodomain comprises a nucleotide sequence that is at least 80% identical to the nucleotide sequence of SEQ ID NO: 32, 34, 82, 84, 88, 90, 94, 96, 100, 102, 44, 46, 56, 58, 68, or 70. In some embodiments, the nucleotide sequence encoding the derivative of the SARS-CoV-2 Omicron virus spike protein ectodomain comprises a nucleotide sequence that is at least 85%, at least 90%, at least 95%, at least 98%, or at least 99% identical to the nucleotide sequence of SEQ ID NO: 32, 34, 82, 84, 88, 90, 94, 96, 100, 102, 44, 46, 56, 58, 68, or 70. In some embodiments, the nucleotide sequence encoding the derivative of the SARS-CoV-2 Omicron virus spike protein ectodomain comprises the nucleotide sequence of SEQ ID NO: 32, 34, 82, 84, 88, 90, 94, 96, 100, 102, 44, 46, 56, 58, 68, or 70. In some embodiments, the SARS-CoV-2 Omicron virus spike protein ectodomain is linked via a linker to the NDV F protein transmembrane and cytoplasmic domains. In some embodiments, the linker comprises the amino acid sequence of SEQ ID NO:24.

In some embodiments, provided herein is a transgene comprising an RNA sequence corresponding to the negative sense of the cDNA sequence of SEQ ID NO:6, 7, 10, 11, 14, 15, 36, 48, or 60, or a nucleotide sequence that is at least 80% identical to the cDNA sequence of SEQ ID NO: 6, 7, 10, 11, 14, 15, 36, 48, or 60. In some embodiments, the transgene comprises an RNA sequence corresponding to the negative sense of the cDNA sequence of a nucleotide sequence that is at least 80% identical to the cDNA sequence of SEQ ID NO: 6, 7, 10, 11, 14, 15, 36, 48, or 60. In some embodiments, the transgene comprises an RNA sequence corresponding to the negative sense of a nucleotide sequence that is at least 85%, at least 90%, at least 95%, at least 98%, or at least 99% identical to the cDNA sequence of SEQ ID NO: 6, 7, 10, 11, 14, 15, 36, 48, or 60. In some embodiments, the transgene comprises an RNA sequence corresponding to the negative sense of the cDNA sequence of SEQ ID NO: 6, 7, 10, 11, 14, 15, 36, 48, or 60.

In some embodiments, provided herein is a transgene comprising an RNA sequence corresponding to the negative sense of the cDNA sequence of SEQ ID NO: 30, 42, 54, 66, 80, 86, 92, 98, 6, 10, 14, 36, 48, 60, or 74, or a nucleotide sequence that is at least 80% identical to the cDNA sequence of SEQ ID NO: 30, 42, 54, 66, 80, 86, 92, 98, 6, 10, 14, 36, 48, 60, or 74. In some embodiments, the transgene comprises an RNA sequence corresponding to the negative sense of the cDNA sequence of a nucleotide sequence that is at least 80% identical to the cDNA sequence of SEQ ID NO: 30, 42, 54, 66, 80, 86, 92, or 98. In some embodiments, the transgene comprises an RNA sequence corresponding to the negative sense of a nucleotide sequence that is at least 85%, at least 90%, at least 95%, at least 98%, or at least 99% identical to the cDNA sequence of SEQ ID NO: 30, 42, 54, 66, 80, 86, 92, 98, 6, 10, 14, 36, 48, 60, or 74. In some embodiments, the transgene comprises an RNA sequence corresponding to the negative sense of the cDNA sequence of SEQ ID NO: 30, 42, 54, 66, 80, 86, 92, 98, 6, 10, 14, 36, 48, 60, or 74.

In some embodiments, provided herein is a transgene comprising an RNA sequence corresponding to the negative sense of the cDNA sequence of SEQ ID NO: 30, 42, 54, 66, 80, 86, 92, 98, 6, 10, 14, 36, 48, 60, or 74, without the signal peptide, or a nucleotide sequence that is at least 80% identical to the cDNA sequence of SEQ ID NO: 30, 42, 54, 66, 80, 86, 92, 98, 6, 10, 14, 36, 48, 60, or 74, without the signal peptide. In some embodiments, the transgene comprises an RNA sequence corresponding to the negative sense of the cDNA sequence of a nucleotide sequence that is at least 80% identical to the cDNA sequence of SEQ ID NO: 30, 42, 54, 66, 80, 86, 92, 98, 6, 10, 14, 36, 48, 60, or 74, without the signal peptide. In some embodiments, the transgene comprises an RNA sequence corresponding to the negative sense of a nucleotide sequence that is at least 85%, at least 90%, at least 95%, at least 98%, or at least 99% identical to the cDNA sequence of SEQ ID NO: 30, 42, 54, 66, 80, 86, 92, 98, 6, 10, 14, 36, 48, 60, or 74, without the signal peptide. In some embodiments, the transgene comprises an RNA sequence corresponding to the negative sense of the cDNA sequence of SEQ ID NO: 30, 42, 54, 66, 80, 86, 92, 98, 6, 10, 14, 36, 48, 60, or 74, without the signal peptide.

In some embodiments, provided herein is a transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises an RNA sequence corresponding to the negative sense of the cDNA sequence of SEQ ID NO: 34, 84, 102, 90, 96, 46, 58, 18, 20, 22, 40, 52, 64, 70, or 78, or a nucleotide sequence that is at least 80% identical to the cDNA sequence of SEQ ID NO: 34, 84, 102, 90, 96, 46, 58, 18, 20, 22, 40, 52, 64, 70, or 78. In some embodiments, the nucleotide sequence encoding the derivative of the SARS-CoV-2 Omicron virus spike protein ectodomain comprises an RNA sequence corresponding to the negative sense of a nucleotide sequence that is at least 80% identical to the cDNA sequence of SEQ ID NO: 34, 84, 102, 90, 96, 46, 58, 18, 20, 22, 40, 52, 64, 70, or 78. In some embodiments, the nucleotide sequence encoding the derivative of the SARS-CoV-2 Omicron virus spike protein ectodomain comprises an RNA sequence corresponding to the negative sense of a nucleotide sequence that is at least 85%, at least 90%, at least 95%, at least 98%, or at least 99% identical to the cDNA sequence of SEQ ID NO: 34, 84, 102, 90, 96, 46, 58, 18, 20, 22, 40, 52, 64, 70, or 78. In some embodiments, the nucleotide sequence encoding the derivative of the SARS-CoV-2 Omicron virus spike protein ectodomain comprises an RNA sequence corresponding to the negative sense of the cDNA sequence of SEQ ID NO: 34, 84, 102, 90, 96, 46, 58, 18, 20, 22, 40, 52, 64, 70, or 78. In some embodiments, the SARS-CoV-2 Omicron virus spike protein ectodomain is linked via a linker to the NDV F protein transmembrane and cytoplasmic domains. In some embodiments, the linker comprises the amino acid sequence of SEQ ID NO:24.

In some embodiments, provided herein is a transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises an RNA sequence corresponding to the negative sense of the cDNA sequence of SEQ ID NO: 32, 82, 100, 88, 94, 44, 56, 38, 50, 62, 68, or 76, or a nucleotide sequence that is at least 80% identical to the cDNA sequence of SEQ ID NO: 32, 82, 100, 88, 94, 44, 56, 38, 50, 62, 68, or 76. In some embodiments, the nucleotide sequence encoding the derivative of the SARS-CoV-2 Omicron virus spike protein ectodomain comprises an RNA sequence corresponding to the negative sense of a nucleotide sequence that is at least 80% identical to the cDNA sequence of SEQ ID NO: 32, 82, 100, 88, 94, 44, 56, 38, 50, 62, 68, or 76. In some embodiments, the nucleotide sequence encoding the derivative of the SARS-CoV-2 Omicron virus spike protein ectodomain comprises an RNA sequence corresponding to the negative sense of a nucleotide sequence that is at least 85%, at least 90%, at least 95%, at least 98%, or at least 99% identical to the cDNA sequence of SEQ ID NO: 32, 82, 100, 88, 94, 44, 56, 38, 50, 62, 68, or 76. In some embodiments, the nucleotide sequence encoding the derivative of the SARS-CoV-2 Omicron virus spike protein ectodomain comprises an RNA sequence corresponding to the negative sense of the cDNA sequence of SEQ ID NO: 34, 84, 102, 90, 96, 46, 58, 18, 20, 22, 40, 52, 64, 70, or 78. In some embodiments, the SARS-CoV-2 Omicron virus spike protein ectodomain is linked via a linker to the NDV F protein transmembrane and cytoplasmic domains. In some embodiments, the linker comprises the amino acid sequence of SEQ ID NO:24.

In some embodiments, the transgene further comprises a Newcastle Disease Virus (NDV) gene start sequence (e.g., SEQ ID NO:27). In some embodiments, the transgene further comprises a Newcastle Disease Virus (NDV) gene end sequence (e.g., SEQ ID NO: 26). In some embodiments, the transgene further comprises SEQ ID NO:26 and 27. In some embodiments, the transgene further comprises SEQ ID NO: 25 or SEQ ID NO:28. In some embodiments, the transgene further comprises SEQ ID NOS: 25 and 28.

In some embodiments, provided herein is a vector comprising a transgene described herein (e.g., in Section 5.1 or 6). The vector may be a plasmid or a viral vector.

In some embodiments, provided herein is a nucleotide sequence comprising a transgene described herein, and (1) a NDV F transcription unit, (2) a NDV NP transcription unit, (3) a NDV M transcription unit, (4) a NDV L transcription unit, (5) a NDV P transcription unit, and (6) a NDV HN transcription unit. In some embodiments, provided herein is a nucleotide sequence comprising a transgene described herein, and (1) a NDV F transcription unit, (2) a NDV NP transcription unit, (3) a NDV M transcription unit, (4) a NDV L transcription unit, (5) a NDV P transcription unit, and (6) a NDV HN transcription unit, wherein the NDV F transcription unit encodes a NDV F protein comprising a leucine to alanine amino acid substitution at the amino residue corresponding to amino acid residue 289 of the LaSota NDV strain. In some embodiments, provided herein is a vector comprising a nucleotide sequence described herein. The vector may be a plasmid or a viral vector.

In another aspect, described herein are recombinant Newcastle disease virus (“NDV”) comprising a packaged genome, wherein the packaged genome comprises a transgene encoding severe acute respiratory syndrome coronavirus 2 (“SARS-CoV-2”) Omicron spike protein or a portion thereof (e.g., ectodomain or receptor binding domain of SARS-CoV-2 Omicron spike protein), or a derivative thereof. In a specific embodiment, described herein are recombinant NDV comprising a packaged genome, wherein the packaged genome comprises a transgene comprising a codon-optimized nucleic acid sequence encoding SARS-CoV-2 Omicron spike protein or portion thereof (e.g., ectodomain or receptor binding domain of SARS-CoV-2 Omicron spike protein), or a derivative thereof. In a specific embodiment, described herein are recombinant NDV comprising a packaged genome, wherein the packaged genome comprises a transgene encoding a chimeric F protein, wherein the chimeric F protein comprises an SARS-CoV-2 Omicron spike protein ectodomain or a derivative thereof and NDV F protein transmembrane and cytoplasmic domains. In some embodiments, the ectodomain of the SARS-CoV-2 Omicron spike protein or derivative thereof is encoded by a codon-optimized nucleic acid sequence.

In some embodiments, provided herein is a recombinant Newcastle disease virus (NDV) comprising a packaged genome, wherein the packaged genome comprises a transgene described herein (e.g., in Section 5.1 or 6). In some embodiments, the NDV virion comprises the chimeric F protein. In some embodiments, provided herein is a recombinant Newcastle disease virus (NDV) comprising a packaged genome, wherein the packaged genome comprises a transgene, wherein the transgene encodes a protein, wherein the protein comprises a derivative of a SARS-CoV-2 Omicron virus spike protein ectodomain, and wherein the derivative of the SARS-CoV-2 Omicron virus spike protein ectodomain comprises the amino acid sequence SEQ ID NO: 35, 85, 103, 91, 97, 47, 59, 19, 21, 23, 41, 53, 65, 71, or 79. In some embodiments, provided herein is a recombinant Newcastle disease virus (NDV) comprising a packaged genome, wherein the packaged genome comprises a transgene, wherein the transgene encodes a protein, wherein the protein comprises a derivative of a SARS-CoV-2 Omicron virus spike protein ectodomain, and wherein the derivative of the SARS-CoV-2 Omicron virus spike protein ectodomain comprises an amino acid sequence at least 80% (e.g., at least 85%, at least 90%, at least 91%, at least 92%, at least 93%, or at least 94%) identical to the amino acid sequence of SEQ ID NO: 35, 85, 103, 91, 97, 47, 59, 19, 21, 23, 41, 53, 65, 71, or 79. In some embodiments, provided herein is a recombinant Newcastle disease virus (NDV) comprising a packaged genome, wherein the packaged genome comprises a transgene, wherein the transgene encodes a protein, wherein the protein comprises a derivative of a SARS-CoV-2 Omicron virus spike protein ectodomain, and wherein the derivative of the SARS-CoV-2 Omicron virus spike protein ectodomain comprises an amino acid sequence at least 90% (e.g., at least 91%, at least 92%, at least 93%, or at least 94%) identical to the amino acid sequence of SEQ ID NO: 35, 85, 103, 91, 97, 47, 59, 19, 21, 23, 41, 53, 65, 71, or 79. In some embodiments, provided herein is a recombinant Newcastle disease virus (NDV) comprising a packaged genome, wherein the packaged genome comprises a transgene, wherein the transgene encodes a protein, wherein the protein comprises a derivative of a SARS-CoV-2 Omicron virus spike protein ectodomain, and wherein the derivative of the SARS-CoV-2 Omicron virus spike protein ectodomain comprises an amino acid sequence at least 95% (e.g., at least 96%, at least 97%, at least 98%, or at least 99%) identical to the amino acid sequence of SEQ ID NO: 35, 85, 103, 91, 97, 47, 59, 19, 21, 23, 41, 53, 65, 71, or 79. In some embodiments, the derivative of the ectodomain comprises: (1) alanine at amino acid residues corresponding to amino acid residues 682 to 685 (RRAR) of the amino acid sequence of SEQ ID NO: 104; (2) proline at amino acid residues corresponding to the following amino acid residues of the amino acid sequence of SEQ ID NO: 104: F817, A892, A899, A942, K986, and V987; and (3) two or more of the following amino acid residues at amino acid positions corresponding to the indicated amino acid positions of the amino acid sequence of SEQ ID NO: 104: 440K, 477N, 505H, 679K, 764K, 796Y, 954H, and/or 969K. In some embodiments, the genome comprises a NDV F transcription unit, a NDV NP transcription unit, a NDV M transcription unit, a NDV L transcription unit, a NDV P transcription unit, and a NDV HN transcription unit. In some embodiments, the genome comprises a NDV F transcription unit, a NDV NP transcription unit, a NDV M transcription unit, a NDV L transcription unit, a NDV P transcription unit, and a NDV HN transcription unit, and wherein the NDV F transcription unit encodes a NDV F protein comprising a leucine to alanine amino acid substitution at the amino residue corresponding to amino acid residue 289 of the LaSota NDV strain. In some embodiments, the transgene is between two NDV transcription units of the packaged genome. In some embodiments, the two transcription units of the packaged genome are the transcription units for the NDV P gene and the NDV M gene. In some embodiments, the two transcription units of the packaged genome are the transcription units for the NDV NP gene and the NDV P gene. In some embodiments, a chimeric F protein or protein encoded by the transgene is incorporated into the NDV virion.

In some embodiments, provided herein is a recombinant NDV comprising a chimeric F protein, wherein the chimeric F protein comprises the amino acid sequence of SEQ ID NO:31, 43, 55, 67, 81, 87, 93, 99, 8, 12, 16, 37, 49, 61, or 75. In some embodiments, the chimeric F protein comprises an amino acid sequence that is at least 80% (e.g., at least 85%, at least 90%, at least 91%, at least 92%, at least 93%, or at least 94%) identical to SEQ ID NO: 31, 43, 55, 67, 81, 87, 93, 99, 8, 12, 16, 37, 49, 61, or 75. In some embodiments, the chimeric F protein comprises an amino acid sequence that is at least 90% (e.g., at least 91%, at least 92%, at least 93%, or at least 94%) identical to SEQ ID NO: 31, 43, 55, 67, 81, 87, 93, 99, 8, 12, 16, 37, 49, 61, or 75. In some embodiments, the chimeric F protein comprises an amino acid sequence that is at least 95%, at least 98%, or at least 99% identical to SEQ ID NO: 31, 43, 55, 67, 81, 87, 93, 99, 8, 12, 16, 37, 49, 61, or 75.

In some embodiments, provided herein is a recombinant NDV comprising a chimeric F protein, wherein the chimeric F protein comprises the amino acid sequence of SEQ ID NO: 31, 43, 55, 67, 81, 87, 93, or 99. In some embodiments, the chimeric F protein comprises an amino acid sequence that is at least 90%, identical to SEQ ID NO: 31, 43, 55, 81, 87, 93, 99, or 67. In some embodiments, the chimeric F protein comprises an amino acid sequence that is at least 95%, at least 98%, or at least 99% identical to SEQ ID NO: 31, 43, 55, 67, 81, 87, 93, or 99. In some embodiments, the chimeric F protein comprises the amino acid sequence of SEQ ID NO: 31, 43, 55, 67, 81, 87, 93, or 99.

In some embodiments, provided herein is a recombinant NDV comprising a chimeric F protein, wherein the chimeric F protein comprises a derivative of a SARS-CoV-2 Omicron virus spike protein ectodomain and NDV F protein transmembrane and cytoplasmic domains, and wherein the derivative of the SARS-CoV-2 Omicron virus spike protein ectodomain comprises an amino acid sequence SEQ ID NO: 35, 85, 103, 91, 97, 47, 59, 19, 21, 23, 41, 53, 65, 71, or 79. In some embodiments, the derivative of the SARS-CoV-2 Omicron virus spike protein ectodomain comprises an amino acid sequence at least 80% (e.g., at least 85%, at least 90%, at least 91%, at least 92%, at least 93%, or at least 94%) identical to the amino acid sequence of SEQ ID NO: 35, 85, 103, 91, 97, 47, 59, 19, 21, 23, 41, 53, 65, 71, or 79. In some embodiments, the derivative of the SARS-CoV-2 Omicron virus spike protein ectodomain comprises an amino acid sequence at least 90% (e.g., at least 91%, at least 92%, at least 93%, or at least 94%) identical to the amino acid sequence of SEQ ID NO: 35, 85, 103, 91, 97, 47, 59, 19, 21, 23, 41, 53, 65, 71, or 79. In some embodiments, the derivative of the SARS-CoV-2 Omicron virus spike protein ectodomain comprises an amino acid sequence at least 95%, at least 98%, or at least 99% identical to the amino acid sequence of SEQ ID NO: 35, 85, 103, 91, 97, 47, 59, 19, 21, 23, 41, 53, 65, 71, or 79. In some embodiments, the SARS-CoV-2 Omicron virus spike protein ectodomain is linked via a linker to the NDV F protein transmembrane and cytoplasmic domains.

In some embodiments, provided herein is a recombinant NDV comprising a chimeric F protein, wherein the chimeric F protein comprises a derivative of a SARS-CoV-2 Omicron virus spike protein ectodomain and NDV F protein transmembrane and cytoplasmic domains, and wherein the derivative of the SARS-CoV-2 Omicron virus spike protein ectodomain comprises an amino acid sequence SEQ ID NO:33, 45, 57, 83, 89, 95, 101, 39, 51, 63, 69, or 77. In some embodiments, the derivative of the SARS-CoV-2 Omicron virus spike protein ectodomain comprises an amino acid sequence at least 80% (e.g., at least 85%, at least 90%, at least 91%, at least 92%, at least 93%, or at least 94%) identical to the amino acid sequence of SEQ ID NO: 33, 45, 57, 83, 89, 95, 101, 39, 51, 63, 69, or 77. In some embodiments, the derivative of the SARS-CoV-2 Omicron virus spike protein ectodomain comprises an amino acid sequence at least 90% (e.g., at least 91%, at least 92%, at least 93%, or at least 94%) identical to the amino acid sequence of SEQ ID NO: 33, 45, 57, 83, 89, 95, 101, 39, 51, 63, 69, or 77. In some embodiments, the derivative of the SARS-CoV-2 Omicron virus spike protein ectodomain comprises an amino acid sequence at least 95%, at least 98%, or at least 99% identical to the amino acid sequence of SEQ ID NO: 33, 45, 57, 83, 89, 95, 101, 39, 51, 63, 69, or 77.

In some embodiments, the derivative of the SARS-CoV-2 Omicron virus spike protein ectodomain comprises an amino acid sequence at least 90% identical to the amino acid sequence of SEQ ID NO: 33, 35, 83, 85, 89, 91, 95, 97, 101, 103, 45, 47, 57, 59, 69, or 71. In some embodiments, the derivative of the SARS-CoV-2 Omicron virus spike protein ectodomain comprises an amino acid sequence at least 90% identical to the amino acid sequence of SEQ ID NO: 33, 35, 83, 85, 89, 91, 95, 97, 101, 103, 45, 47, 57, 59, 69, or 71. In some embodiments, the derivative of the SARS-CoV-2 Omicron virus spike protein ectodomain comprises an amino acid sequence at least 90% identical to the amino acid sequence of SEQ ID NO: 33, 35, 83, 85, 89, 91, 95, 97, 101, or 103. In some embodiments, the derivative of the SARS-CoV-2 Omicron virus spike protein ectodomain comprises an amino acid sequence at least 95%, at least 98%, or at least 99% identical to the amino acid sequence of SEQ ID NO: 33, 35, 83, 85, 89, 91, 95, 97, 101, 103, 45, 47, 57, 59, 69, or 71. In some embodiments, the derivative of the SARS-CoV-2 Omicron virus spike protein ectodomain comprises an amino acid sequence at least 95%, at least 98%, or at least 99% identical to the amino acid sequence of SEQ ID NO: 33, 35, 83, 85, 89, 91, 95, 97, 101, or 103. In some embodiments, the derivative of the SARS-CoV-2 Omicron virus spike protein ectodomain comprises the amino acid sequence of SEQ ID NO: 33, 35, 83, 85, 89, 91, 95, 97, 101, 103, 45, 47, 57, 59, 69, or 71. In some embodiments, the derivative of the SARS-CoV-2 Omicron virus spike protein ectodomain comprises the amino acid sequence of SEQ ID NO: 33, 35, 83, 85, 89, 91, 95, 97, 101, or 103. In some embodiments, the SARS-CoV-2 Omicron virus spike protein ectodomain is linked via a linker to the NDV F protein transmembrane and cytoplasmic domains.

In some embodiments, provided herein is a recombinant NDV comprising a protein, wherein the protein comprises a derivative of a SARS-CoV-2 Omicron virus spike protein ectodomain, and wherein the derivative of the SARS-CoV-2 Omicron virus spike protein ectodomain comprises the amino acid sequence of SEQ ID NO: 35, 85, 103, 91, 97, 47, 59, 19, 21, 23, 41, 53, 65, 71, or 79. In some embodiments, provided herein is a recombinant NDV comprising a protein, wherein the protein comprises a derivative of a SARS-CoV-2 Omicron virus spike protein ectodomain, and wherein the derivative of the SARS-CoV-2 Omicron virus spike protein ectodomain comprises an amino acid sequence at least 80% (e.g., at least 85%, at least 90%, at least 91%, at least 92%, at least 93%, or at least 94%) identical to the amino acid sequence of SEQ ID NO: 35, 85, 103, 91, 97, 47, 59, 19, 21, 23, 41, 53, 65, 71, or 79. In some embodiments, provided herein is a recombinant NDV comprising a protein, wherein the protein comprises a derivative of a SARS-CoV-2 Omicron virus spike protein ectodomain, and wherein the derivative of the SARS-CoV-2 Omicron virus spike protein ectodomain comprises an amino acid sequence at least 90% (e.g., at least 91%, at least 92%, at least 93%, or at least 94%) identical to the amino acid sequence of SEQ ID NO: 35, 85, 103, 91, 97, 47, 59, 19, 21, 23, 41, 53, 65, 71, or 79. In some embodiments, provided herein is a recombinant NDV comprising a protein, wherein the protein comprises a derivative of a SARS-CoV-2 Omicron virus spike protein ectodomain, and wherein the derivative of the SARS-CoV-2 Omicron virus spike protein ectodomain comprises an amino acid sequence at least 95%, at least 96%, at least 97%, at least 98%, or at least 99% identical to the amino acid sequence of SEQ ID NO: 35, 85, 103, 91, 97, 47, 59, 19, 21, 23, 41, 53, 65, 71, or 79. In some embodiments, the derivative of the ectodomain comprises an amino acid sequence at least 90% identical to the amino acid sequence of SEQ ID NO: 35, 85, 103, 91, 97, 47, 59, 19, 21, 23, 41, 53, 65, 71, or 79, and wherein the derivative of the ectodomain comprises: (1) alanine at amino acid residues corresponding to amino acid residues 682 to 685 (RRAR) of the amino acid sequence of SEQ ID NO:104; (2) proline at amino acid residues corresponding to the following amino acid residues of the amino acid sequence of SEQ ID NO: 104: F817, A892, A899, A942, K986, and V987; and (3) two or more of the following amino acid residues at amino acid positions corresponding to the indicated amino acid positions of the amino acid sequence of SEQ ID NO: 104: 440K, 477N, 505H, 679K, 764K, 796Y, 954H, and/or 969K

In some embodiments, the recombinant NDV comprises an NDV backbone which is lentogenic. In some embodiments, the recombinant NDV comprises an NDV backbone of LaSota strain. In some embodiments, the recombinant NDV comprises an NDV backbone of Hitchner B1 strain.

In another aspect, provided herein are compositions (e.g., immunogenic compositions) comprising a recombinant NDV described herein. The composition (e.g., immunogenic composition) may be monovalent, bivalent, or multivalent. In some embodiments, the immunogenic composition is monovalent. In some embodiments, the recombinant NDV described herein is inactivated. In some embodiments, an immunogenic composition described herein further comprises an adjuvant (e.g., an adjuvant described herein). The immunogenic composition may be used to induce an immune response, immunize a subject against SARS-CoV-2, and/or the prevent of COVID-19.

In some embodiments, provided herein is an immunogenic composition comprising a polynucleotide described herein. In some embodiments, the immunogenic composition described herein further comprises an adjuvant (e.g., an adjuvant described herein). The polynucleotide may be RNA, DNA, or a combination thereof. The polynucleotide may comprise naturally occurring nucleotides or analogs thereof. The immunogenic composition may be used to induce an immune response, immunize a subject against SARS-CoV-2, and/or the prevent of COVID-19.

In some embodiments, provided herein is an immunogenic composition comprising a recombinant protein described herein. In some embodiments, the immunogenic composition described herein further comprises an adjuvant (e.g., an adjuvant described herein). The immunogenic composition may be used to induce an immune response, immunize a subject against SARS-CoV-2, and/or the prevent of COVID-19.

In some embodiments, provided herein is an immunogenic composition comprising a vector described herein. In some embodiments, the immunogenic composition described herein further comprises an adjuvant (e.g., an adjuvant described herein). The immunogenic composition may be used to induce an immune response, immunize a subject against SARS-CoV-2, and/or the prevent of COVID-19.

In another aspect, the recombinant NDV described herein and the immunogenic compositions described herein are for use in inducing an immune response, immunizing a subject against SARS-CoV-2, and/or the prevention of COVID-19. In some embodiments, the recombinant NDV described herein and the immunogenic compositions described herein are for use in preventing moderate or severe COVID-19. In some embodiments, provided herein is a method for inducing an immune response to SARS-CoV-2 Omicron spike protein, comprising administering an immunogenic composition described herein to a subject. In some embodiments, provided herein is a method for preventing COVID-19, comprising administering an immunogenic composition described herein to a subject. In some embodiments, provided herein is a method for preventing severe COVID-19, comprising administering an immunogenic composition described herein to a subject. In some embodiments, provided herein is a method for immunizing a subject against SARS-CoV-2, comprising administering an immunogenic composition described herein to a subject. In specific embodiments, the composition is administered to the subject intranasally or intramuscularly. In a specific embodiment, the subject is a human. In some embodiments, the subject has been previously vaccinated with a COVID-19 vaccine. In some embodiments, the subject is administered at least one booster of the immunogenic composition.

In another aspect, provided herein are kits. In some embodiments, provided herein is a kit comprising a transgene described herein. In some embodiments, provided herein is a kit comprising a polynucleotide described herein. In some embodiments, provided herein is a kit comprising a nucleotide sequence described herein. In some embodiments, provided herein is a vector described herein. In some embodiments, provided herein is a kit comprising a recombinant protein described herein. In some embodiments, provided herein is a recombinant NDV described herein. In some embodiments, provided herein is a kit comprising an immunogenic composition described herein.

In another aspect, provided herein is a cell(s) (e.g., a cell line) or an embryonated egg (e.g., a chicken embryonated egg) comprising a transgene described herein, a polynucleotide described herein, or a nucleotide sequence described herein. In some embodiments, provided herein is a cell(s) (e.g., a cell line) or an embryonated egg (e.g., a chicken embryonated egg) comprising a transgene described herein. In some embodiments, provided herein is a cell(s) (e.g., a cell line) or an embryonated egg (e.g., a chicken embryonated egg) comprising a polynucleotide described herein. In another aspect, provided herein is a cell(s) (e.g., a cell line) or an embryonated egg (e.g., embryonated chicken egg) comprising a vector described herein. In another aspect, provided herein is a cell(s) (e.g., a cell line) or an embryonated egg (e.g., chicken embryonated egg) comprising a recombinant NDV described herein. In another aspect, provided herein is a cell(s) (e.g., a cell line) or an embryonated egg (e.g., a chicken embryonated egg) expressing a protein described herein. In some embodiments, the cell(s) is in vitro or ex vivo.

In another aspect, provided herein is a method for propagating a recombinant NDV described herein, the method comprising culturing a cell(s) (e.g., cell line) or an embryonated egg described herein. In some embodiments, the method further comprises isolating the recombinant NDV from the cell(s) (e.g., cell line) or embryonated egg.

In another aspect, provided herein is a method for detecting the presence of antibody specific to SARS-CoV-2 Omicron spike protein, comprising contacting a specimen with a recombinant NDV described herein in an immunoassay. In another aspect, provided herein is a method for detecting the presence of antibody specific to SARS-CoV-2 Omicron spike protein, comprising contacting a specimen with a recombinant protein described herein in an immunoassay. In another aspect, provided herein is a method for detecting the presence of antibody specific to SARS-CoV-2 Omicron spike protein, comprising contacting a specimen with a vector expressing a protein described herein in an immunoassay. In some embodiments, the specimen is a biological specimen. In some embodiments, the biological specimen is blood, plasma or sera from a subject. In some embodiments, the subject is human. In some embodiments, the specimen is an antibody or antisera.

### 3.1 Terminology

As used herein, the term “about” or “approximately” when used in conjunction with a number refers to any number within 1, 5 or 10% of the referenced number, including the referenced number.

The phrase “amino acid modifications” includes amino acid substitutions, amino acid deletions, and/or amino acid insertions.

As used herein, the terms “antibody” and “antibodies” refer to molecules that contain an antigen binding site, e.g., immunoglobulins. Antibodies include, but are not limited to, monoclonal antibodies, bispecific antibodies, multispecific antibodies, human antibodies, humanized antibodies, synthetic antibodies, chimeric antibodies, polyclonal antibodies, single domain antibodies, camelized antibodies, single-chain Fvs (scFv), single chain antibodies, Fab fragments, F(ab′) fragments, disulfide-linked bispecific Fvs (sdFv), intrabodies, and anti-idiotypic (anti-Id) antibodies (including, e.g., anti-Id and anti-anti-Id antibodies to antibodies), and epitope-binding fragments of any of the above. In particular, antibodies include immunoglobulin molecules and immunologically active fragments of immunoglobulin molecules. Immunoglobulin molecules can be of any type (e.g., IgG, IgE, IgM, IgD, IgA and IgY), class (e.g., IgG1, IgG2, IgG3, IgG4, IgA1 and IgA2) or subclass.

As used herein, the term “elderly human” refers to a human 65 years or older.

As used herein, the term “human adult” refers to a human that is 18 years or older.

As used herein, the term “human child” refers to a human that is 1 year to 18 years old.

As used herein, the term “human toddler” refers to a human that is 1 year to 3 years old.

As used herein, the term “human infant” refers to a newborn to 1 year old year human.

As used herein, the phrases “IFN deficient systems” or “IFN-deficient substrates” refer to systems, e.g., cells, cell lines and animals, such as mice, chickens, turkeys, rabbits, rats, horses etc., which do not produce one, two or more types of IFN, or do not produce any type of IFN, or produce low levels of one, two or more types of IFN, or produce low levels of any IFN (i.e., a reduction in any IFN expression of 5-10%, 10-20%, 20-30%, 30-40%, 40-50%, 50-60%, 60-70%, 70-80%, 80-90% or more when compared to IFN-competent systems under the same conditions), do not respond or respond less efficiently to one, two or more types of IFN, or do not respond to any type of IFN, have a delayed response to one, two or more types of IFN, are deficient in the activity of antiviral genes induced by one, two or more types of IFN, or induced by any type of IFN, or any combination thereof.

As used herein, the terms “subject” or “patient” are used interchangeably. As used herein, the terms “subject” and “subjects” refers to an animal. In some embodiments, the subject is a mammal including a non-primate (e.g., a camel, donkey, zebra, bovine, horse, horse, cat, dog, rat, and mouse) and a primate (e.g., a monkey, chimpanzee, and a human). In some embodiments, the subject is a non-human mammal. In certain embodiments, the subject is a pet (e.g., dog or cat) or farm animal (e.g., a horse, pig or cow). In specific embodiments, the subject is a human. In certain embodiments, the mammal (e.g., human) is 4 to 6 months old, 6 to 12 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100 years old. In specific embodiments, the subject is an animal that is not avian.

As used herein, the term “in combination” in the context of the administration of a therapy(ies) to a subject, refers to the use of more than one therapy. The use of the term “in combination” does not restrict the order in which therapies are administered to a subject. A first therapy can be administered prior to, concomitantly with, or subsequent to the administration of a second therapy to a subject.

As used herein, the terms “SARS-CoV-2 spike protein” and “spike protein of SARS-CoV-2” includes a SARS-CoV-2 spike protein known to those of skill in the art. See, e.g., GenBank Accession Nos. MN908947.3, MT447160, MT44636, MT446360, MT444593, MT444529, MT370887, and MT334558 for examples of amino acid sequences of SARS-CoV-2 spike protein and nucleotide sequences encoding SARS-CoV-2 spike protein. A typical spike protein comprises domains known to those of skill in the art including an S1 domain, a receptor binding domain, an S2 domain, a transmembrane domain and a cytoplasmic domain. See, e.g., Wrapp et al., 2020, Science 367:1260-1263 and Duan et al., 2020, Front. Immunol., Vol. 11, Article 576622 for a description of SARS-CoV-2 spike protein (in particular, the structure of such protein). The spike protein may be characterized has having a signal peptide, a receptor binding domain, an ectodomain, an S1 domain, an S2 domain, and a transmembrane and endodomain (or cytoplasmic).

As used herein, the terms “spike protein of an Omicron variant of a SARS-CoV-2”, “SARS-CoV-2 Omicron spike protein”, “SARS-CoV-2 Omicron variant spike protein” and “spike protein of SARS-CoV-2 Omicron variant” includes a SARS-CoV-2 Omicron variant spike protein known to those of skill in the art. See, e.g., GISAID Accession Numbers EPI_ISL_6640917, EPI_ISL_6640916, EPI_ISL_6640919, EPI_ISL_7580387, and EPI_ISL_12920491. In specific embodiments, the Omicron variant is of the BA. 1 lineage. In specific embodiments, the Omicron variant is of the BA.2 lineage. In specific embodiments, the Omicron variant is of the BA.4/5 lineage. In specific embodiments, the Omicron variant is of the BA.5 lineage. In specific embodiments, the spike protein of BA.5 comprises the amino acid sequence of the spike protein of the BA.5 strain hCoV-19/Albania/280808/2022 found at GISAID Accession ID: EPI_ISL_17295779. In specific embodiments, the Omicron variant is of the BQ.1.1 lineage. In specific embodiments, the spike protein of BQ.1.1 comprises the amino acid sequence of the spike protein of the BQ.1.1 strain hCoV-19/Canada/QC-L00595284001/2023 found at GISAID Accession ID: EPI_ISL_17321793. In specific embodiments, the Omicron variant is of the XBB.1.5 lineage. In specific embodiments, the spike protein of XBB.1.5 comprises the amino acid sequence of the spike protein of the XBB. 1.5 strain hCoV-19/Spain/CT-HUB07938/2023 found at GISAID Accession ID: EPI_ISL_17321709.

As used herein, the term “Wuhan strain” refers to the SARS-CoV-2 strain referred to by one of skill in the art as the Wuhan strain. See, e.g., GenBank Accession No. MN908947.3. In specific embodiments, the spike protein of the Wuhan strain comprises the amino acid sequence of the spike protein found at GenBank Accession No. MN908947.3. SEQ ID NO: 104 reproduces the spike protein found at GenBank Accession No. MN908947.3. SEQ ID NO: 105 reproduces the spike protein found at found at GenBank Accession No. MN908947.3, without the signal peptide.

As used herein, the terms “therapies” and “therapy” can refer to any protocol(s), method(s), agent(s) or a combination thereof that can be used in the treatment or prevention of COVID-19, or vaccination. In certain embodiments, the term “therapy” refers to a recombinant NDV described herein. In other embodiments, the term “therapy” refers to an agent that is not a recombinant NDV described herein.

The term “and/or” as a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophilic (Cys, Ser, Thr), acidic (Asp, Glu), basic (Asn, Gln, His, Lys, Arg), conformation disruptors (Gly, Pro) and aromatic (Trp, Tyr, Phe).

### 4. BRIEF DESCRIPTION OF THE FIGURES

FIGS. 1A-1B. Characterization of NDV-HXP-S variants. NDV-HXP-S variants were rescued by reverse genetics as previously described (Ayllon et al., 2013, J Vis Exp. (80): 50830). Cells were co-transfected with the expression plasmid required for replication and transcription of the NDV viral genome (NP, P, and L), together with the full-length NDV cDNA. After 1 day, transfected cells were co-cultured with DF-1 cells. After 2 or 3 days, the co-culture cell supernatants were inoculated into eight- or nine-day-old specific pathogen free (SPF) embryonated chicken eggs. Antigen identity was confirmed by biochemical methods and sequencing. The genetic stability of the recombinant viruses was evaluated across multiples passages on ten days old-SPF embryonated chicken eggs. The spike protein in the allantoic fluid were detected by western blot using an anti-spike 2B3E5 mouse monoclonal antibody. FIG. 1A shows the western blot results of allantoic fluid from embryonated eggs infected with NDV-HXP-S (Wuhan) or NDV-HXP-S Omicron BA.1. FIG. 1B shows the western blot results of allantoic fluid from embryonated eggs infected with NDV-HXP-S (Wuhan) or NDV-HXP-S Omicron BA.1 (H655_delCSV687I). H655: In the Omicron spike protein 655Y was changed back to H655. delCSV687I: Deletion of 679KSHRRARS686 and changed V687 to 687I.

FIGS. 2A-2C. NDV-HXP-S variants were rescued by reverse genetics as previously described (Ayllon et al., 2013, J Vis Exp. (80): 50830). Cells were co-transfected with the expression plasmid required for replication and transcription of the NDV viral genome (NP, P, and L), together with the full-length NDV cDNA. After 1 day, transfected cells were co-cultured with DF-1 cells. After 2 or 3 days, the co-culture cell supernatants were inoculated into eight- or nine-day-old specific pathogen free (SPF) embryonated chicken eggs. Antigen identity was confirmed by biochemical methods and sequencing. The genetic stability of the recombinant viruses was evaluated across multiples passages on ten days old-SPF embryonated chicken eggs. The spike protein in the allantoic fluid were detected by western blot using an anti-spike 2B3E5 mouse monoclonal antibody. FIG. 2A shows the western blot results of allantoic fluid from embryonated eggs infected with NDV-HXP-S (Wuhan) or NDV-HXP-S Omicron BA.1. FIG. 2B shows the western blot results of allantoic fluid from embryonated eggs infected with NDV-HXP-S Omicron BA.1 (H655_delCSV687I).

FIG. 3. NDV-HXP-S variants were rescued by reverse genetics as previously described (Ayllon et al., 2013, J Vis Exp. (80): 50830). Cells were co-transfected with the expression plasmid required for replication and transcription of the NDV viral genome (NP, P, and L), together with the full-length NDV cDNA. After 1 day, transfected cells were co-cultured with DF-1 cells. After 2 or 3 days, the co-culture cell supernatants were inoculated into eight- or nine-day-old specific pathogen free (SPF) embryonated chicken eggs. Antigen identity was confirmed by biochemical methods and sequencing. The genetic stability of the recombinant viruses was evaluated across multiples passages on ten days old-SPF embryonated chicken eggs. The spike protein in the allantoic fluid were detected by western blot using an anti-spike 2B3E5 mouse monoclonal antibody.

FIGS. 4A-4B. Characterization of NDV-HXP-S variants. NDV-HXP-S variants were rescued by reverse genetics as previously described (Ayllon et al., 2013, J Vis Exp. (80): 50830). Cells were co-transfected with the expression plasmid required for replication and transcription of the NDV viral genome (NP, P, and L), together with the full-length NDV cDNA. After 1 day, transfected cells were co-cultured with DF-1 cells. After 2 or 3 days, the co-culture cell supernatants were inoculated into eight- or nine-day-old specific pathogen free (SPF) embryonated chicken eggs. Antigen identity was confirmed by biochemical methods and sequencing. The genetic stability of the recombinant viruses was evaluated across multiples passages on ten days old-SPF embryonated chicken eggs. The spike protein in the allantoic fluid were detected by western blot using an anti-spike 2B3E5 mouse monoclonal antibody. FIG. 4A shows the western blot results of allantoic fluid from embryonated eggs infected with NDV-HXP-S (Wuhan) or NDV-HXP-S Omicron BA.2. FIG. 4B shows the western blot results of allantoic fluid from embryonated eggs infected with NDV-HXP-S (Wuhan) or NDV-HXP-S Omicron BA.2 SSS (S371, S372, and S375).

FIGS. 5A-5C. Characterization of NDV-HXP-S variants. NDV-HXP-S variants were rescued by reverse genetics as previously described (Ayllon et al., 2013, J Vis Exp. (80): 50830). Cells were co-transfected with the expression plasmid required for replication and transcription of the NDV viral genome (NP, P, and L), together with the full-length NDV cDNA. After 1 day, transfected cells were co-cultured with DF-1 cells. After 2 or 3 days, the co-culture cell supernatants were inoculated into eight- or nine-day-old specific pathogen free (SPF) embryonated chicken eggs. Antigen identity was confirmed by biochemical methods and sequencing. The genetic stability of the recombinant viruses was evaluated across multiples passages on ten days old-SPF embryonated chicken eggs. The spike protein in the allantoic fluid were detected by western blot using an anti-spike 2B3E5 mouse monoclonal antibody. FIG. 5A shows the western blot results of allantoic fluid from embryonated eggs infected with NDV-HXP-S (Wuhan), NDV-HXP-S Omicron BA.5 SSS (S371, S372, and S375), or NDV-HXP-S Omicron BA.5. FIG. 5B shows the western blot results of allantoic fluid from embryonated eggs infected with NDV-HXP-S (Wuhan), NDV-HXP-S Omicron BA.5 SSS (S371, S372, S375, and G446), NDV-HXP-S Omicron BA.5 SSS (S371, S372, S375, and F486), NDV-HXP-S Omicron BA.5 SSS (S371, S372, S375, and Add69-70), NDV-HXP-S Omicron BA.5 SSS L452 (S371, S372, S375, and L452). FIG. 5C shows the western blot results of allantoic fluid from embryonated eggs infected with NDV-HXP-S (Wuhan), NDV-HXP-S Omicron BA.5 SSS L452 (S371, S372, S375, and L452).

FIGS. 6A-6C. Characterization of NDV-HXP-S variants. NDV-HXP-S variants were rescued by reverse genetics as previously described (Ayllon et al., 2013, J Vis Exp. (80): 50830). Cells were co-transfected with the expression plasmid required for replication and transcription of the NDV viral genome (NP, P, and L), together with the full-length NDV cDNA. After 1 day, transfected cells were co-cultured with DF-1 cells. After 2 or 3 days, the co-culture cell supernatants were inoculated into eight- or nine-day-old specific pathogen free (SPF) embryonated chicken eggs. Antigen identity was confirmed by biochemical methods and sequencing. The genetic stability of the recombinant viruses was evaluated across multiples passages on ten days old-SPF embryonated chicken eggs. The spike protein in the allantoic fluid were detected by western blot using an anti-spike 2B3E5 mouse monoclonal antibody. FIG. 6A shows the western blot results of allantoic fluid from embryonated eggs infected with NDV-HXP-S (Wuhan) or NDV-HXP-S Omicron BQ1.1. FIG. 6B shows a Coomassie Blue staining of purified virus from allanotic fluid from embryonated eggs infected with NDV-HXP-S (Wuhan) or NDV-HXP-S Omicron BQ1.1. FIG. 6C shows the western blot results of allantoic fluid from embryonated eggs infected with NDV-HXP-S (Wuhan) or NDV-HXP-S Omicron XBB1.5.

FIGS. 7A-7C. NDV-HXP-S variants were rescued by reverse genetics as previously described (Ayllon et al., 2013, J Vis Exp. (80): 50830). Cells were co-transfected with the expression plasmid required for replication and transcription of the NDV viral genome (NP, P, and L), together with the full-length NDV cDNA. After 1 day, transfected cells were co-cultured with DF-1 cells. After 2 or 3 days, the co-culture cell supernatants were inoculated into eight- or nine-day-old specific pathogen free (SPF) embryonated chicken eggs. Antigen identity was confirmed by biochemical methods and sequencing. The genetic stability of the recombinant viruses was evaluated across multiples passages on ten days old-SPF embryonated chicken eggs. The spike protein in the allantoic fluid were detected by western blot using an anti-spike 2B3E5 mouse monoclonal antibody. FIG. 7A shows the western blot results of allantoic fluid from embryonated eggs infected with NDV-HXP-S (Wuhan) or NDV-HXP-S Omicron BA.1. FIG. 7B shows the western blot results of allantoic fluid from embryonated eggs infected with NDV-HXP-S (Wuhan) or NDV-HXP-S Omicron BA.1 (S371, S373, S375). FIG. 7C shows the western blot results of allantoic fluid from embryonated eggs infected with NDV-HXP-S (Wuhan) or NDV-HXP-S Omicron BA.1 (S371, S375).

FIGS. 8A-8C. Immunization studies in mice to compare the immunogenicity of the cleaved BA.1 WT spike and the stabilized BA.1 SSS spike. Female BALB/c mice were immunized intranasally with live vaccine of NDV-HXP-S (Wuhan), NDV-HXP-S (BA.1 WT), and NDV-HXP-S (BA.1 SSS) twice with a 4-week interval (FIG. 8A). Four weeks after the first and second dose, serum IgG against ancestral spike protein, ancestral RBD protein, BA. 1 spike protein, BA. 1 RBD protein, BA.4/5 spike protein, and BA.4/5 RBD protein were measured by ELISAs (FIGS. 8B and 8C).

FIGS. 9A-9C. Immunization studies in mice to investigate humoral responses using NDV-HXP-S ancestral and BA.1 SSS as a booster vaccine. Female BALB/c mice were vaccinated with NDV-HXP-S Wuhan strain twice with a 3-week interval between the first and second dose. Approximately 5 months later, a third booster with either the same ancestral NDV-HXP-S Wuhan vaccine or the NDV-HXP-S BA.1 SSS vaccine was given. Each vaccination was administered intranasally at the same dose to each mouse. Antibodies induced by the following vaccinations were measured: two vaccinations of the NDV-HXP-S Wuhan (2×NDV-HXP-S), three vaccinations of NDV-HXP-S Wuhan (3×NDV-HXP-S), two vaccinations of NDV-HXP-S Wuhan followed by NDV-HXP-S BA.1 SSS booster, and two vaccinations of the vector (2×NDV WT) (FIG. 9A). Serum IgG titers were measured for all four conditions against the Wuhan spike, BA. 1 spike, Wuhan RBD, BA.1 RBD as well as the vector (inactivated whole virion of NDV WT) by ELISAs (FIGS. 9B and 9C).

### 5. DETAILED DESCRIPTION

Provided herein are transgenes encoding a chimeric F protein, recombinant NDV comprising such a transgene, and recombinant NDV comprising such a chimeric F protein, wherein the chimeric F protein comprises a SARS-CoV-2 Omicron spike protein ectodomain or a derivative thereof, and NDV F protein transmembrane and cytoplasmic domains. The disclosure is based, in part, upon the surprising discovery that maintaining certain amino acid residues corresponding to certain amino acid residues of the spike protein of GenBank Accession No. MN908947.3 in a derivative of the Omicron spike protein variant BA. 1 ectodomain prevents cleavage of the spike protein. For example, the disclosure is based, in part, upon the surprising discovery that maintaining serines at amino acid positions corresponding to amino acid residues 371 and 375 (or amino acid residues 371, 373, and 373) of the spike protein of GenBank Accession No. MN908947.3 in a derivative of the Omicron spike protein variant BA. 1 ectodomain prevents cleavage of the spike protein. See, e.g., Examples 2 and 5. The disclosure is also based, in part, upon the surprising discovery that maintaining serines at amino acid positions corresponding to amino acid residues 371, 373, and 373 of the spike protein of GenBank Accession No. MN908947.3 in a derivative of the Omicron spike protein variant BA.2 ectodomain prevents cleavage of the spike protein. See, e.g., Examples 3 and 4. The disclosure is also based, in part, upon the surprising discovery that maintaining serines at amino acid positions corresponding to amino acid residues 371, 373, and 375 of the spike protein of GenBank Accession No. MN908947.3, and maintaining leucine at the amino acid position corresponding to 452 of the spike protein of GenBank Accession No. MN908947.3 in a derivative of the Omicron spike protein variant BA. 1 ectodomain prevents cleavage of the spike protein. See, e.g., Examples 4. Thus, in some embodiments, a derivative of a SARS-CoV-2 Omicron spike protein ectodomain includes a serine at amino acid positions corresponding to amino acid positions 371 and 375 of SEQ ID NO: 104, or a serine at amino acid positions corresponding to amino acid positions 371, 373, and 375 of SEQ ID NO:104. In some embodiments, a derivative of a SARS-CoV-2 Omicron spike protein ectodomain includes a serine at amino acid positions corresponding to amino acid positions 371, 373, and 375 of SEQ ID NO: 104, and a leucine at the amino acid position corresponding to amino acid position 452 of SEQ ID NO:104.

### 5.1 Recombinant Newcastle Disease Virus

In one aspect, provided herein are transgenes comprising a nucleotide sequence encoding a chimeric F protein, wherein the chimeric F protein comprises a SARS-CoV-2 Omicron spike protein ectodomain or a derivative thereof, and NDV F protein transmembrane and cytoplasmic domains. The recombinant NDV may be administered as a live virus or an inactivated virus.

### 5.1.1 NDV

Newcastle disease virus (NDV) is a member of the Avulavirus genus in the Paramyxoviridae family, which has been shown to infect a number of avian species (Alexander, DJ (1988). Newcastle disease, Newcastle disease virus—an avian paramyxovirus. Kluwer Academic Publishers: Dordrecht, The Netherlands. pp 1-22). NDV possesses a single-stranded RNA genome in negative sense and does not undergo recombination with the host genome or with other viruses (Alexander, DJ (1988). Newcastle disease, Newcastle disease virus—an avian paramyxovirus. Kluwer Academic Publishers: Dordrecht, The Netherlands. pp 1-22). The genomic RNA contains genes in the order of 3′-NP-P-M-F-HN-L-5′. Two additional proteins, V and W, are produced by NDV from the P gene by alternative mRNAs that are generated by RNA editing. The genomic RNA also contains a leader sequence at the 3′ end.

The structural elements of the virion include the virus envelope which is a lipid bilayer derived from the cell plasma membrane. The glycoprotein, hemagglutinin-neuraminidase (HN) protrudes from the envelope allowing the virus to contain both hemagglutinin (e.g., receptor binding/fusogenic) and neuraminidase activities. The fusion glycoprotein (F), which also interacts with the viral membrane, is first produced as an inactive precursor, then cleaved post-translationally to produce two disulfide linked polypeptides. The active F protein is involved in penetration of NDV into host cells by facilitating fusion of the viral envelope with the host cell plasma membrane. The matrix protein (M), is involved with viral assembly, and interacts with both the viral membrane as well as the nucleocapsid proteins.

The main protein subunit of the nucleocapsid is the nucleocapsid protein (NP) which confers helical symmetry on the capsid. In association with the nucleocapsid are the P and L proteins. The phosphoprotein (P), which is subject to phosphorylation, is thought to play a regulatory role in transcription, and may also be involved in methylation, phosphorylation and polyadenylation. The L gene, which encodes an RNA-dependent RNA polymerase, is required for viral RNA synthesis together with the P protein. The L protein, which takes up nearly half of the coding capacity of the viral genome is the largest of the viral proteins, and plays an important role in both transcription and replication.

Any NDV type or strain may be serve as the “backbone” that is engineered to comprise a transgene described herein, including, but not limited to, naturally-occurring strains, variants or mutants, mutagenized viruses, reassortants and/or genetically engineered viruses. See, e.g., Section 5.1.2 and Section 6 for examples of transgenes. In a specific embodiment, a transgene described herein is incorporated into the genome of a lentogenic NDV. In another specific embodiment, a transgene described herein is incorporated into the genome of NDV strain LaSota. In another embodiment, a transgene described herein is incorporated into the genome of NDV Hitchner B1 strain. In some embodiments, a lentogenic strain other than NDV Hitchner B1 strain is used as the backbone into which a nucleotide sequence may be incorporated. The transgene may be incorporated into the NDV genome between two transcription units (e.g., between the NDV M and P transcription units, between the NDV NP and P transcription units, or between the NDV HN and L transcription units).

In a specific embodiment, a NDV that is engineered to comprise a transgene described herein is a naturally-occurring strain. Specific examples of NDV strains include, but are not limited to, Hitchner B1 strain (see, e.g., GenBank No. AF309418 or NC_002617) and LaSota strain (see, e.g., GenBank Nos. AY845400, AF07761.1 and JF950510.1 and GI No. 56799463). In a specific embodiment, the NDV that is engineered to comprises a transgene described herein is the Hitchner B1 strain. In another embodiment, the NDV that is engineered to comprise a transgene described herein is a B1 strain as identified by GenBank No. AF309418 or NC_002617. In a specific embodiment, the nucleotide sequence of the Hitchner B1 genome comprises an RNA sequence corresponding to the negative sense of the cDNA sequence set forth in SEQ ID NO:2. In another specific embodiment, the NDV that is engineered to comprise a transgene described herein is the LaSota strain. In another embodiment, the NDV that is engineered to comprise a transgene described herein is a LaSota strain as identified by AY845400, AF07761.1 or JF950510.1. In a specific embodiment, the nucleotide sequence of the LaSota genome comprises an RNA sequence corresponding to the negative sense of the cDNA sequence set forth in SEQ ID NO:1. In another specific embodiment, the nucleotide sequence of the LaSota genome comprises an RNA sequence corresponding to the negative sense of the cDNA sequence set forth in SEQ ID NO: 3. One skilled in the art will understand that the NDV genomic RNA sequence is an RNA sequence corresponding to the negative sense of a cDNA sequence encoding the NDV genome. Thus, any program that generates converts a nucleotide sequence to its reverse complement sequence may be utilized to convert a cDNA sequence encoding an NDV genome into the genomic RNA sequence (see, e.g., www.bioinformatics.org/sms/rev_comp.html, www.fr33.net/seqedit.php, and DNAStar). Accordingly, the nucleotide sequences provided in Tables 1-4, infra, may be readily converted to the negative-sense RNA sequence of the NDV genome by one of skill in the art.

In a specific embodiment, the NDV that is engineered to comprise a transgene described herein comprises a genome encoding an NDV F protein in which a leucine amino acid residue at amino acid position 289 of NDV F protein is substituted for alanine (as described by, e.g., Sergel et al., 2000, Journal of Virology 74:5101-5107). In another specific embodiment, the NDV that is engineered to comprise a transgene described herein comprises a genome encoding an NDV F protein in which a leucine amino acid residue at amino acid position 289 of NDV F protein (as counted by the LaSota strain F protein) is substituted for alanine. In another specific embodiment, the NDV that is engineered to comprise a transgene described herein comprises a nucleotide sequence encoding an NDV F protein in which leucine at the amino acid position corresponding to amino acid residue 289 of LaSota NDV F protein is substituted for alanine. In another specific embodiment, the NDV that is engineered to comprise a transgene described herein comprises a nucleotide sequence encoding an NDV F protein in which leucine at the amino acid residue 289 of LaSota NDV F protein is substituted for alanine. In another specific embodiment, the NDV that is engineered to comprise a transgene described herein is of the LaSota strain (e.g., GenBank Accession Nos. AY845400, AF07761.1 or JF950510.1) and the genome of the LaSota strain encodes an NDV F protein in which a leucine amino acid residue at amino acid position 289 of NDV F protein is substituted for alanine. In another specific embodiment, the NDV that is engineered to comprise a transgene described herein is of the LaSota strain (e.g., GenBank Accession Nos. AY845400, AF07761.1 or JF950510.1) and the genome of the LaSota strain comprises a nucleotide sequence encoding LaSota NDV F protein in which leucine at amino acid residue 289 of the NDV F protein (as counted by the LaSota strain F protein) is substituted for alanine. In another specific embodiment, the NDV that is engineered to comprise a transgene described herein is of the Hitchner B1 strain (e.g., GenBank No. AF309418 or NC_002617) and the genome of the Hitchner B1 strain encodes an NDV F protein in which a leucine amino acid residue at amino acid position 289 of NDV F protein (as counted by the LaSota strain F protein) is substituted for alanine.

In some embodiments, the NDV that is engineered to comprise a transgene described herein is of the Fuller strain. In certain embodiments, the NDV genome that is engineered to comprise a transgene described herein is of the Ulster strain. In some embodiments, the NDV that is engineered to comprise a transgene described herein is of the Roakin strain. In certain embodiments, the NDV that is engineered to comprise a transgene described herein is of the Komarov strain. In some embodiments, the NDV that is engineered to comprise a transgene described herein is of the Roakin strain. In certain embodiments, the NDV that is engineered to comprise a transgene described herein is of the r73T-RI 16 virus.

In specific embodiments, the NDV that is engineered to comprise a transgene described herein is not pathogenic in birds as assessed by a technique known to one of skill. In certain specific embodiments, the NDV that is engineered to comprise a transgene described herein is not pathogenic as assessed by intracranial injection of 1-day-old chicks with the virus, and disease development and death as scored for 8 days. In some embodiments, the NDV that is engineered to comprise a transgene described herein has an intracranial pathogenicity index of less than 0.7, less than 0.6, less than 0.5, less than 0.4, less than 0.3, less than 0.2 or less than 0.1. In certain embodiments, the NDV that is engineered to comprise a transgene described herein has an intracranial pathogenicity index of zero. See, e.g., OIE Terrestrial Manual 2012, Chapter 2.3.14, entitled “Newcastle Disease (Infection With Newcastle Disease Virus) for a description of this assay, which is found at the following website www.oie.int/fileadmin/Home/eng/Health_standards/tahm/2.03.14 NEWCASTLE_DIS.pdf, which is incorporated herein by reference in its entirety.

In certain embodiments, the NDV that is engineered to comprise a transgene described herein is a mesogenic strain that has been genetically engineered so as not be a considered pathogenic in birds as assessed by techniques known to one skilled in the art.

In preferred embodiments, the NDV that is engineered to comprise a transgene described herein is non-pathogenic in humans. In preferred embodiments, the NDV that is engineered to comprise a transgene described herein is non-pathogenic in human and avians. In certain embodiments, the NDV that is engineered to comprise a transgene described herein is attenuated such that the NDV remains, at least partially, infectious and can replicate in vivo, but only generate low titers resulting in subclinical levels of infection that are non-pathogenic (see, e.g., Khattar et al., 2009, J. Virol. 83:7779-7782). Such attenuated NDVs may be especially suited for embodiments wherein the virus is administered to a subject in order to act as an immunogen, e.g., a live vaccine. The viruses may be attenuated by any method known in the art. In a specific embodiment, the genome of NDV comprises sequences necessary for infection and replication of the virus such that progeny is produced and the infection level is subclinical. In certain embodiments, NDV is attenuated by introducing one, two, or more mutations (e.g., amino acid substitutions) in the NDV V protein.

In some embodiments, provided herein is a recombinant NDV comprising a genome comprising a nucleotide sequence described herein or polynucleotide sequence described herein.

In a specific embodiment, provided herein is a nucleotide sequence comprising: (1) an NDV F transcription unit, (2) an NDV NP transcription unit, (3) an NDV P transcription unit, (4) an NDV M transcription unit, (5) an NDV HN transcription unit, (6) an NDV L transcription unit, and (7) a transgene described herein. In certain embodiments, the NDV transcription units are LaSota NDV transcription units. In a specific embodiment, provided herein is a nucleotide sequence comprising: (1) an NDV F transcription unit, (2) an NDV NP transcription unit, (3) an NDV P transcription unit, (4) an NDV M transcription unit, (5) an NDV HN transcription unit, (6) an NDV L transcription unit, and (7) a transgene described herein, wherein the NDV F transcription unit encodes an NDV F protein with an amino acid substitution of leucine to alanine at the amino acid residue corresponding to amino acid position 289 of LaSota NDV F protein. In another specific embodiment, provided herein is a nucleotide sequence comprising (1) an NDV F transcription unit, (2) an NDV NP transcription unit, (3) an NDV P transcription unit, (4) an NDV M transcription unit, (5) an NDV HN transcription unit, (6) an NDV L transcription unit, and (7) a transgene described herein, wherein the NDV F transcription unit encodes an NDV F protein with an amino acid substitution of leucine to alanine at amino acid position 289 of LaSota NDV F protein. In certain embodiments, the NDV transcription units are LaSota NDV transcription units. In certain embodiments, the nucleotide sequence is part of a vector (e.g., a plasmid). In specific embodiments, the nucleotide sequence is isolated.

In a specific embodiment, provided herein is a polynucleotide sequence comprising: (1) a nucleotide sequence encoding NDV F, (2) a nucleotide sequence encoding NDV NP, (3) a nucleotide sequence encoding NDV P, (4) a nucleotide sequence encoding NDV M, (5) a nucleotide sequence encoding NDV HN, (6) a nucleotide sequence encoding NDV L, and (7) a transgene described herein. In another specific embodiment, provided herein is a polynucleotide sequence comprising: (1) a nucleotide sequence encoding NDV F, (2) a nucleotide sequence encoding NDV NP, (3) a nucleotide sequence encoding NDV P, (4) a nucleotide sequence encoding NDV M, (5) a nucleotide sequence encoding NDV HN, (6) a nucleotide sequence encoding NDV L, and (7) a transgene described herein, wherein the NDV F comprises an amino acid substitution of leucine to alanine at the amino acid position corresponding to amino acid residue 289 of LaSota NDV F. In another specific embodiment, provided herein is a polynucleotide sequence comprising: (1) a nucleotide sequence encoding NDV F, (2) a nucleotide sequence encoding NDV NP, (3) a nucleotide sequence encoding NDV P, (4) a nucleotide sequence encoding NDV M, (5) a nucleotide sequence encoding NDV HN, (6) a nucleotide sequence encoding NDV L, and (7) a transgene described herein, wherein the NDV F comprises an amino acid substitution of leucine to alanine at the amino acid position 289 of LaSota NDV F. In certain embodiments, the NDV proteins are LaSota NDV proteins. In another specific embodiment, provided herein is a polynucleotide sequence comprising a nucleotide sequence of an NDV genome known in the art or described (see, e.g., Section 5.1 or the Example below; see also SEQ ID NO: 1, 2 or 3) and a transgene described herein. In certain embodiments, the nucleic acid sequence is part of a vector (e.g., a plasmid). In a specific embodiment, the polynucleotide sequence is isolated.

In specific embodiments, a polynucleotide sequence described herein, a nucleic acid sequence described herein, or nucleotide sequence described herein is a recombinant polynucleotide sequence described herein, recombinant nucleic acid sequence described herein, or recombinant nucleotide sequence. In certain embodiments, a polynucleotide sequence described herein, a nucleotide sequence described herein, or nucleic acid sequence described herein may be a DNA molecule (e.g., cDNA), an RNA molecule (e.g., mRNA), or a combination of a DNA and RNA molecule. In some embodiments, a polynucleotide sequence described herein, nucleotide sequence described herein, or nucleic acid sequence described herein may comprise analogs of DNA or RNA molecules. Such analogs can be generated using, for example, nucleotide analogs, which include, but are not limited to, inosine, methylcytosine, pseudouridine, or tritylated bases. Such analogs can also comprise DNA or RNA molecules comprising modified backbones that lend beneficial attributes to the molecules such as, for example, nuclease resistance or an increased ability to cross cellular membranes. The polynucleotide sequences, nucleic acid sequences, or nucleotide sequences can be single-stranded, double-stranded, may contain both single-stranded and double-stranded portions, and may contain triple-stranded portions. In a specific embodiment, a polynucleotide sequence described herein, nucleotide sequence described herein, or nucleic acid sequence described herein is a negative sense single-stranded RNA. In another specific embodiment, a polynucleotide sequence described herein, a nucleotide sequence described herein, or nucleic acid sequence described herein is a positive sense single-stranded RNA. In another specific embodiment, a polynucleotide sequence described herein, nucleotide sequence described herein, or nucleic acid sequence described herein is a cDNA.

5.1.2 SARS-CoV-2 Variant Spike Protein/Chimeric F Protein with the SARS-CoV-2 Variant Spike Protein Ectodomain or Derivative Thereof

In a specific embodiment, provided herein is a transgene comprising a nucleotide sequence encoding a protein comprising a SARS-CoV-2 Omicron spike protein or portion thereof (e.g., ectodomain, S1 domain, S2 domain, or receptor binding domain of the SARS-CoV-2 Omicron spike protein). See, e.g., Section 5.1.1, supra, for types and strains of NDV that may be used. The transgene encoding a SARS-CoV-2 Omicron variant spike protein or portion thereof (e.g., ectodomain, S1 domain, S2 domain, or receptor binding domain of the SARS-CoV-2 Omicron variant spike protein) may inserted into any NDV type or strain (e.g., NDV LaSota strain). In a specific embodiment, a transgene encoding a SARS-CoV-2 Omicron variant spike protein or portion thereof (e.g., ectodomain, S1 domain, S2 domain, or receptor binding domain of the SARS-CoV-2 Omicron variant spike protein) is incorporated into the genome of any NDV type or strain (e.g., NDV LaSota strain). In a specific embodiment, the SARS-CoV-2 Omicron variant is of the BA.1 sublineage. In a specific embodiment, the SARS-CoV-2 Omicron variant is of the BA.2 sublineage. In a specific embodiment, the SARS-CoV-2 Omicron variant is of the BA.4/5 sublineage. In a specific embodiment, the SARS-CoV-2 Omicron variant is of the BQ1.1. In a specific embodiment, the SARS-CoV-2 Omicron variant is of the XBB1.5. See, e.g., Section 3.1 for exemplary sequences for SARS-CoV-2 Omicron variant spike proteins or portion thereof (e.g., ectodomain, S1 domain, S2 domain, or receptor binding domain of the SARS-CoV-2 Omicron variant spike protein) and exemplary nucleic acid sequences encoding SARS-CoV-2 Omicron variant spike protein or portion thereof (e.g., ectodomain, S1 domain, S2 domain, or receptor binding domain of the SARS-CoV-2 Omicron variant spike protein). One of skill in the art would be able to use such sequence information to produce a transgene for incorporation into the genome of any NDV type or strain. Given the degeneracy of the nucleic acid code, there are a number of different polynucleotide sequences that may encode the same SARS-CoV-2 Omicron variant spike protein or portion thereof (e.g., ectodomain, S1 domain, S2 domain or receptor binding domain of the SARS-CoV-2 Omicron variant spike protein). In a specific embodiment, a transgene encoding a SARS-CoV-2 Omicron variant spike protein or portion thereof (e.g., ectodomain, S1 domain, S2 domain, or receptor binding domain of the SARS-CoV-2 Omicron variant spike protein) is codon optimized. See, e.g., Section 5.1.4, infra, for a discussion regarding codon optimization. In certain embodiments, the transgene encoding a SARS-CoV-2 Omicron variant spike protein or a portion thereof (e.g., ectodomain, S1 domain, S2 domain, or receptor binding domain of SARS-CoV-2 Omicron variant spike protein) without the SARS-CoV-2 Omicron variant spike protein signal peptide. The transgene encoding a SARS-CoV-2 Omicron variant spike protein or portion thereof (e.g., ectodomain, S1 domain, S2 domain, or receptor binding domain of the SARS-CoV-2 Omicron variant spike protein) may be incorporated between any two NDV transcription units (e.g., between the NDV P and M transcription units, between the NDV NP and P transcription units, or between the HN and L transcription units).

In certain embodiments, a portion of a SARS-CoV-2 Omicron variant spike protein comprises the receptor binding domain of the SARS-CoV-2 Omicron variant spike protein. In some embodiments, a portion of a SARS-CoV-2 Omicron variant spike protein comprises the receptor binding domain of the SARS-CoV-2 Omicron variant spike protein and 5, 10, 15, 20, 30, 40, 50, 75 or more amino acid residues to N-terminus of the receptor binding domain of the SARS-CoV-2 Omicron variant spike protein, or 5, 10, 15, 20, 30, 40, 50, 75 or more amino acid residues C-terminus to the receptor binding domain of the SARS-CoV-2 Omicron variant spike protein, or 5, 10, 15, 20, 30, 40, 50, 75 or more amino acid residues N-terminus to the receptor binding domain of the SARS-CoV-2 Omicron variant spike protein and 5, 10, 15, 20, 30, 40, 50, 75 or more amino acid residues C-terminus to the receptor binding domain of the SARS-CoV-2 Omicron variant spike protein. In some embodiments, a portion of a SARS-CoV-2 Omicron variant spike protein comprises the receptor binding domain of the SARS-CoV-2 Omicron variant spike protein and 5 to 25, 5 to 50, 25 to 50, 25 to 75, or 50 to 75 amino acid residues N-terminus to the receptor binding domain of the SARS-CoV-2 Omicron variant spike protein, 5 to 25, 5 to 50, 25 to 50, 25 to 75, or 50 to 75 amino acid residues C-terminus to the receptor binding domain of the SARS-CoV-2 Omicron variant spike protein, or 5 to 25, 5 to 50, 25 to 50, 25 to 75, or 50 to 75 amino acid residues N-terminus to the receptor binding domain of SARS-CoV-2 Omicron variant spike protein and 5 to 25, 5 to 50, 25 to 50, 25 to 75, or 50 to 75 amino acid residues C-terminus to the receptor binding domain of the SARS-CoV-2 Omicron variant spike protein.

In certain embodiments, a portion of a SARS-CoV-2 Omicron variant spike protein comprises the S1 domain of the SARS-CoV-2 Omicron variant spike protein. In some embodiments, a portion of a SARS-CoV-2 Omicron variant spike protein comprises the S1 domain of the SARS-CoV-2 Omicron variant spike protein and 5, 10, 15, 20, 30, 40, 50, 75 or more amino acid residues to N-terminus of the S1 domain of the SARS-CoV-2 Omicron variant spike protein, or 5, 10, 15, 20, 30, 40, 50, 75 or more amino acid residues C-terminus to the S1 domain of the SARS-CoV-2 Omicron variant spike protein, or 5, 10, 15, 20, 30, 40, 50, 75 or more amino acid residues N-terminus to the S1 domain of the SARS-CoV-2 Omicron variant spike protein and 5, 10, 15, 20, 30, 40, 50, 75 or more amino acid residues C-terminus to the S1 domain of the SARS-CoV-2 Omicron variant spike protein. In some embodiments, a portion of a SARS-CoV-2 Omicron variant spike protein comprises the S1 domain of the SARS-CoV-2 Omicron variant spike protein and 5 to 25, 5 to 50, 25 to 50, 25 to 75, or 50 to 75 amino acid residues N-terminus to the S1 domain of the SARS-CoV-2 Omicron variant spike protein, 5 to 25, 5 to 50, 25 to 50, 25 to 75, or 50 to 75 amino acid residues C-terminus to the S1 domain of the SARS-CoV-2 Omicron variant spike protein, or 5 to 25, 5 to 50, 25 to 50, 25 to 75, or 50 to 75 amino acid residues N-terminus to the S1 domain of SARS-CoV-2 Omicron variant spike protein and 5 to 25, 5 to 50, 25 to 50, 25 to 75, or 50 to 75 amino acid residues C-terminus to the S1 domain of the SARS-CoV-Omicron variant spike protein.

In certain embodiments, a portion of a SARS-CoV-2 Omicron variant spike protein comprises the S2 domain of the SARS-CoV-2 Omicron variant spike protein. In some embodiments, a portion of a SARS-CoV-2 Omicron variant spike protein comprises the S2 domain of the SARS-CoV-2 spike protein and 5, 10, 15, 20, 30, 40, 50, 75 or more amino acid residues to N-terminus of the S2 domain of the SARS-CoV-2 Omicron variant spike protein, or 5, 10, 15, 20, 30, 40, 50, 75 or more amino acid residues C-terminus to the S2 domain of the SARS-CoV-2 Omicron variant spike protein, or 5, 10, 15, 20, 30, 40, 50, 75 or more amino acid residues N-terminus to the S2 domain of the SARS-CoV-2 Omicron variant spike protein and 5, 10, 15, 20, 30, 40, 50, 75 or more amino acid residues C-terminus to the S2 domain of the SARS-CoV-2 Omicron variant spike protein. In some embodiments, a portion of a SARS-CoV-2 Omicron variant spike protein comprises the S2 domain of the SARS-CoV-2 Omicron variant spike protein and 5 to 25, 5 to 50, 25 to 50, 25 to 75, or 50 to 75 amino acid residues N-terminus to the S2 domain of the SARS-CoV-2 Omicron variant spike protein, 5 to 25, 5 to 50, 25 to 50, 25 to 75, or 50 to 75 amino acid residues C-terminus to the S2 domain of the SARS-CoV-2 Omicron variant spike protein, or 5 to 25, 5 to 50, 25 to 50, 25 to 75, or 50 to 75 amino acid residues N-terminus to the S2 domain of SARS-CoV-2 Omicron variant spike protein and 5 to 25, 5 to 50, 25 to 50, 25 to 75, or 50 to 75 amino acid residues C-terminus to the S2 domain of the SARS-CoV-2 Omicron variant spike protein.

In certain embodiments, a portion of a SARS-CoV-2 Omicron variant spike protein comprises the S1 domain and S2 domain of the SARS-CoV-2 Omicron variant spike protein. In some embodiments, a portion of a SARS-CoV-2 Omicron variant spike protein comprises the S1 domain and S2 domain of the SARS-CoV-2 Omicron variant spike protein and 5, 10, 15, 20, 30, 40, 50, 75 or more amino acid residues to N-terminus of the S1 domain of the SARS-CoV-2 Omicron variant spike protein, or 5, 10, 15, 20, 30, 40, 50, 75 or more amino acid residues C-terminus to the S2 domain of the SARS-CoV-2 Omicron variant spike protein, or 5, 10, 15, 20, 30, 40, 50, 75 or more amino acid residues N-terminus to the S1 domain of the SARS-CoV-2 Omicron variant spike protein and 5, 10, 15, 20, 30, 40, 50, 75 or more amino acid residues C-terminus to the S2 domain of the SARS-CoV-2 Omicron variant spike protein. In some embodiments, a portion of a SARS-CoV-2 Omicron variant spike protein comprises the S1 domain and S2 domain of the SARS-CoV-2 Omicron variant spike protein and 5 to 25, 5 to 50, 25 to 50, 25 to 75, or 50 to 75 amino acid residues N-terminus to the S1 domain of the SARS-CoV-2 Omicron variant spike protein, 5 to 25, 5 to 50, 25 to 50, 25 to 75, or 50 to 75 amino acid residues C-terminus to the S2 domain of the SARS-CoV-2 Omicron variant spike protein, or 5 to 25, 5 to 50, 25 to 50, 25 to 75, or 50 to 75 amino acid residues N-terminus to the S1 domain of SARS-CoV-2 Omicron variant spike protein and 5 to 25, 5 to 50, 25 to 50, 25 to 75, or 50 to 75 amino acid residues C-terminus to the S2 domain of the SARS-CoV-Omicron variant spike protein.

In certain embodiments, a portion of a SARS-CoV-2 Omicron variant spike protein comprises the ectodomain of the SARS-CoV-2 Omicron variant spike protein. In some embodiments, a portion of a SARS-CoV-2 Omicron variant spike protein comprises the ectodomain of the SARS-CoV-2 Omicron variant spike protein and 5, 10, 15, 20, 30, 40, 50, 75 or more amino acid residues to N-terminus of the ectodomain of the SARS-CoV-2 Omicron variant spike protein, or 5, 10, 15, 20, 30, 40, 50, 75 or more amino acid residues C-terminus to the ectodomain of the SARS-CoV-2 Omicron variant spike protein, or 5, 10, 15, 20, 30, 40, 50, 75 or more amino acid residues N-terminus to the ectodomain of the SARS-CoV-2 Omicron variant spike protein and 5, 10, 15, 20, 30, 40, 50, 75 or more amino acid residues C-terminus to the ectodomain of the SARS-CoV-2 Omicron variant spike protein. In some embodiments, a portion of a SARS-CoV-2 Omicron variant spike protein comprises the ectodomain of the SARS-CoV-2 Omicron variant spike protein and 5 to 25, 5 to 50, 25 to 50, 25 to 75, or 50 to 75 amino acid residues N-terminus to the ectodomain of the SARS-CoV-2 Omicron variant spike protein, 5 to 25, 5 to 50, 25 to 50, 25 to 75, or 50 to 75 amino acid residues C-terminus to the ectodomain of the SARS-CoV-2 Omicron variant spike protein, or 5 to 25, 5 to 50, 25 to 50, 25 to 75, or 50 to 75 amino acid residues N-terminus to the ectodomain of SARS-CoV-2 Omicron variant spike protein and 5 to 25, 5 to 50, 25 to 50, 25 to 75, or 50 to 75 amino acid residues C-terminus to the ectodomain of the SARS-CoV-2 Omicron variant spike protein.

In certain embodiments, a portion of a SARS-CoV-2 Omicron variant spike protein comprises 200, 220, 222, 250, 300, 350, 400, or more amino acid residues. In some embodiments, a portion of a SARS-CoV-2 Omicron variant spike protein comprises 450, 500, 550, 600, 650, 700, 750, 800, 850, 900, 950, 1000, 1100, 1200 or more amino acid residues. In specific embodiments, the amino acid residues are contiguous.

In another embodiment, described herein is a transgene comprising a nucleotide sequence encoding a full-length SARS-CoV-2 Omicron variant spike protein or a fragment thereof. In another embodiment, described herein is a transgene comprising a nucleotide sequence encoding a portion of a SARS-CoV-2 Omicron variant spike protein. In certain embodiments, the protein further comprises a domain(s) that facilitate purification, folding and cleavage of portions of a polypeptide. For example, a His tag (His-His-His-His-His-His (SEQ ID NO:72)), FLAG epitope or other purification tag can facilitate purification of the protein provided herein. In some embodiments, the His tag has the sequence, (His) n, wherein n is 2, 3, 4, 5, 6, 7, 8, 9, 10, 11, 12, 13, 14, 15, 16, 17, 18, 19, 20 or greater. In certain embodiments, a fragment of the SARS-CoV-2 Omicron variant spike protein is at least 1000, 1025, 1075, 1100, 1125, 1150, 1200 or 1215 amino acid residues in length.

In another embodiment, provided herein is a transgene comprising a nucleotide sequence that is at least 85%, at least 90%, at least 95%, at least 96%, at least 97%, at least 98% or at least 99% identical to the nucleotide sequence of a SAR-CoV-2 Omicron variant spike protein, or a portion thereof (e.g., ectodomain, S1 domain, S2 domain, or a receptor binding domain), or a fragment thereof. In another embodiment, provided herein is a transgene comprising a nucleotide sequence that is at least 95%, at least 96%, at least 97%, at least 98% or at least 99% identical to the nucleotide sequence of a SAR-CoV-2 Omicron variant spike protein, or a portion thereof (e.g., ectodomain, S1 domain, S2 domain, or a receptor binding domain), or a fragment thereof. In another embodiment, provided herein is a transgene comprising a nucleotide sequence that is at least 96%, at least 97%, at least 98% or at least 99% identical to the nucleotide sequence of a SAR-CoV-2 Omicron variant spike protein, or a portion thereof (e.g., ectodomain, S1 domain, S2 domain, or a receptor binding domain), or a fragment thereof. Methods/techniques known in the art may be used to determine sequence identity (see, e.g., “Best Fit” or “Gap” program of the Sequence Analysis Software Package, version 10; Genetics Computer Group, Inc.). In certain embodiments, the protein further comprises one or more polypeptide domains. The one or more polypeptide domains may be at the C-terminus or N-terminus, or C-terminus and N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His (SEQ ID NO: 72)), FLAG epitope or other purification tag can facilitate purification of the protein provided herein. In some embodiments, the His tag has the sequence, (His) n, wherein n is 2, 3, 4, 5, 6, 7, 8, 9, 10, 11, 12, 13, 14, 15, 16, 17, 18, 19, 20 or greater. In one embodiment, the His tag has the sequence (His) n, wherein n is 6 (SEQ ID NO:72). In certain embodiments, a fragment of the SARS-CoV-2 spike protein is at least 250, at least 500, at least 750, at least 1000, at least 1025, at least 1075, at least 1100, at least 1125, at least 1150, at least 1175, at least 1200, or at least 1215 amino acid residues in length.

Techniques known to one of skill in the art can be used to determine the percent identity between two amino acid sequences or between two nucleotide sequences. Generally, to determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions X 100%). In one embodiment, the two sequences are the same length. In a certain embodiment, the percent identity is determined over the entire length of an amino acid sequence or nucleotide sequence. In some embodiments, the length of sequence identity comparison may be over the full-length of the two sequences being compared (e.g., the full-length of a gene coding sequence, or a fragment thereof). In some embodiments, a fragment of a nucleotide sequence is at least 25, at least 50, at least 75, or at least 100 nucleotides. Similarly, “percent sequence identity” may be readily determined for amino acid sequences, over the full-length of a protein, or a fragment thereof. In some embodiments, a fragment of a protein comprises at least 20, at least 30, at least 40, at least 50 or more contiguous amino acids of the protein. In certain embodiments, a fragment of a protein comprises at least 75, at least 100, at least 125, at least 150 or more contiguous amino acids of the protein.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to nucleic acid molecules described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another preferred, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

In another embodiment, provided herein is a transgene comprising a nucleotide sequence encoding a protein comprising (or consisting of) a SARS-CoV-2 Omicron variant spike protein with 1, 2, 3, 4, 5, 6, 7, 8, 9, 10, 11, 12, 13, 14, 15, 16, 17, 18, 19, 20, or more amino acids substituted with another amino acid (e.g., a conservative amino acid substitution). In another embodiment, provided herein is a transgene comprising a nucleotide sequence encoding a protein comprising (or consisting of) a SARS-CoV-2 Omicron variant spike protein with 1, 2, 3, 4, 5, 6, 7, 8, 9, 10, 11, or 12 amino acids at the C-terminus substituted with another amino acid (e.g., a conservative amino acid substitution). In another embodiment, provided herein is a transgene comprising a nucleotide sequence encoding a protein comprising (or consisting of) a SARS-CoV-2 Omicron variant spike protein with 1, 2, 3, 4, 5, 6, 7, 8, 9, 10, 11, or 12 amino acids at the N-terminus substituted with another amino acid (e.g., a conservative amino acid substitution). In another embodiment, provided herein is a transgene comprising a nucleotide sequence encoding a protein comprising (or consisting of) a SARS-CoV-2 Omicron variant spike protein with 1, 2, 3, 4, 5, 6, 7, 8, 9, 10, 11, or 12 amino acids at the N-terminus substituted with another amino acid (e.g., a conservative amino acid substitution) and 1, 2, 3, 4, 5, 6, 7, 8, 9, 10, 11, or 12 amino acids at the C-terminus substituted with another amino acid (e.g., a conservative amino acid substitution). In a specific embodiment, the N-terminus is the first 100 amino acid residues of the SARS-CoV-2 Omicron variant spike protein. In a specific embodiment, the C-terminus is the last 100 amino acid residues of the SARS-CoV-2 Omicron variant spike protein. In specific embodiments, the SARS-CoV-2 Omicron variant spike protein is the mature form of the protein. In other embodiments, the SARS-CoV-2 Omicron variant spike protein is the immature form of the protein. In certain embodiments, the protein further comprise one or more polypeptide domains. The one or more polypeptide domains may be at the C-terminus or N-terminus. In a specific embodiment, the one or more polypeptide domains are at the C-terminus Useful polypeptide domains include domains that facilitate purification, folding and cleavage of portions of a polypeptide. For example, a His tag (His-His-His-His-His-His (SEQ ID NO:72)), FLAG epitope or other purification tag can facilitate purification of the protein provided herein. In some embodiments, the His tag has the sequence, (His) n, wherein n is 2, 3, 4, 5, 6, 7, 8, 9, 10, 11, 12, 13, 14, 15, 16, 17, 18, 19, 20 or greater. In one embodiment, the His tag has the sequence (His) n, wherein n is 6 (SEQ ID NO:72).

In another embodiment, provided herein is a transgene comprising a nucleotide sequence encoding a protein comprising (or consisting of) a SARS-CoV-2 Omicron variant spike protein with 1, 2, 3, 4, 5, 6, 7, 8, 9, 10, 11, or 12 amino acids deleted. In another embodiment, provided herein is a transgene comprising a nucleotide sequence encoding a protein comprising (or consisting of) a SARS-CoV-2 Omicron variant spike protein with 1, 2, 3, 4, 5, 6, 7, 8, 9, 10, 11, or 12 amino acids deleted from the C-terminus. In another embodiment, provided herein is a transgene comprising a nucleotide sequence encoding a protein comprising (or consisting of) a SARS-CoV-2 Omicron variant spike protein with 1, 2, 3, 4, 5, 6, 7, 8, 9, 10, 11, or 12 amino acids deleted from the N-terminus. In a specific embodiment, the N-terminus is the first 100 amino acid residues of the SARS-CoV-2 Omicron variant spike protein. In a specific embodiment, the C-terminus is the last 100 amino acid residues of the SARS-CoV-2 Omicron variant spike protein. In specific embodiments, the SARS-CoV-2 Omicron variant spike protein is the mature form of the protein. In other embodiments, the SARS-CoV-2 Omicron variant spike protein is the immature form of the protein. In certain embodiments, the protein further comprises one or more polypeptide domains. The one or more polypeptide domains may be at the C-terminus or N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His (SEQ ID NO:72)), FLAG epitope or other purification tag can facilitate purification of the protein provided herein. In some embodiments, the His tag has the sequence, (His) n, wherein n is 2, 3, 4, 5, 6, 7, 8, 9, 10, 11, 12, 13, 14, 15, 16, 17, 18, 19, 20 or greater. In one embodiment, the His tag has the sequence (His) n, wherein n is 6 (SEQ ID NO:72).

In another embodiment, provided herein is a transgene comprising a nucleotide sequence encoding a protein comprising (or consisting of) a SARS-CoV-2 Omicron variant spike protein with 1, 2, 3, 4, 5, 6, 7, 8, 9, 10, 11, 12, 13, 14, 15, 16, 17, 18, 19, 20, or more mutations (e.g., amino acid substitutions, amino acid deletions, amino acid additions, or a combination thereof). In another embodiment, provided herein is a transgene comprising a nucleotide sequence encoding a protein comprising (or consisting of) a SARS-CoV-2 Omicron variant spike protein with 1, 2, 3, 4, 5, 6, 7, 8, 9, 10, 11, or 12 amino acids deleted and 1, 2, 3, 4, 5, 6, 7, 8, 9, 10, 11 or 12 amino acid substitutions. In a specific embodiment, the N-terminus is the first 100 amino acid residues of the SARS-CoV-2 Omicron variant spike protein. In a specific embodiment, the C-terminus is the last 100 amino acid residues of the SARS-CoV-2 Omicron variant spike protein. In specific embodiments, the SARS-CoV-2 Omicron variant spike protein is the mature form of the protein. In other embodiments, the SARS-CoV-2 Omicron variant spike protein is the immature form of the protein. In certain embodiments, the protein further comprises one or more polypeptide domains. The one or more polypeptide domains may be at the C-terminus, N-terminus, or the C-terminus and N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His (SEQ ID NO:72)), FLAG epitope or other purification tag can facilitate purification of the protein provided herein. In some embodiments, the His tag has the sequence, (His) n, wherein n is 2, 3, 4, 5, 6, 7, 8, 9, 10, 11, 12, 13, 14, 15, 16, 17, 18, 19, 20 or greater. In one embodiment, the His tag has the sequence (His) n, wherein n is 6 (SEQ ID NO:72).

In another embodiment, described herein is a transgene comprising a nucleotide sequence encoding a protein comprising (or consisting of) the receptor binding domain of a SARS-CoV-2 Omicron variant spike protein. In certain embodiments, protein further comprise one or more polypeptide domains. Useful polypeptide domains include domains that facilitate purification, folding and cleavage of portions of a polypeptide. For example, a His tag (His-His-His-His-His-His (SEQ ID NO:72)), FLAG epitope or other purification tag can facilitate purification of the protein provided herein. In some embodiments, the His tag has the sequence, (His) n, wherein n is 2, 3, 4, 5, 6, 7, 8, 9, 10, 11, 12, 13, 14, 15, 16, 17, 18, 19, 20 or greater. In a specific embodiment, a protein comprises or consists of the receptor binding domain of a SARS-CoV-2 Omicron variant spike protein and a His tag (e.g., a (His) n, where n is 6 (SEQ ID NO:72)). In certain embodiments, a protein comprising (or consisting) of the receptor binding domain of a SARS-CoV-2 Omicron variant spike protein is a secreted polypeptide. In a specific embodiment, when designing a protein comprising SARS-CoV-2 Omicron variant spike protein receptor binding domain, care is taken to maintain the stability of the resulting protein.

In another embodiment, provided herein is a transgene comprising a nucleotide sequence encoding a protein comprising (or consisting of) a SARS-CoV-2 Omicron variant spike protein receptor binding domain with 1, 2, 3, 4, 5, 6, 7, 8, 9, 10, 11, or 12 amino acids substituted with another amino acid (e.g., a conservative amino acid substitution). In another embodiment, provided herein is a transgene comprising a nucleotide sequence encoding a protein comprising (or consisting of) a SARS-CoV-2 Omicron variant spike protein receptor binding domain with 1, 2, 3, 4, 5, 6, 7, 8, 9, 10, 11, or 12 amino acids at the C-terminus of the receptor binding domain substituted with another amino acid (e.g., a conservative amino acid substitution). In another embodiment, provided herein is a transgene comprising a nucleotide sequence encoding a protein comprising (or consisting of) a SARS-CoV-2 Omicron variant spike protein receptor binding domain with 1, 2, 3, 4, 5, 6, 7, 8, 9, 10, 11, or 12 amino acids at the N-terminus of the receptor binding domain substituted with another amino acid (e.g., a conservative amino acid substitution). In another embodiment, provided herein is a transgene comprising a nucleotide sequence encoding a protein comprising (or consisting of) a SARS-CoV-2 Omicron variant spike protein receptor binding domain with 1, 2, 3, 4, 5, 6, 7, 8, 9, 10, 11, or 12 amino acids at the N-terminus of the receptor binding domain substituted with another amino acid (e.g., a conservative amino acid substitution) and 1, 2, 3, 4, 5, 6, 7, 8, 9, 10, 11, or 12 amino acids at the C-terminus of the receptor binding domain substituted with another amino acid (e.g., a conservative amino acid substitution). In a specific embodiment, the N-terminus is the first 25 amino acid residues of the receptor binding domain of the SARS-CoV-2 Omicron variant spike protein. In a specific embodiment, the C-terminus is the last 25 amino acid residues of the receptor binding domain of the SARS-CoV-2 Omicron variant spike protein. In certain embodiments, the protein further comprises one or more polypeptide domains. The one or more polypeptide domains may be at the C-terminus, N-terminus, C-terminus and N-terminus. In a specific embodiment, the one or more polypeptide domains are at the C-terminus Useful polypeptide domains include domains that facilitate purification, folding and cleavage of portions of a polypeptide. For example, a His tag (His-His-His-His-His-His (SEQ ID NO:72)), FLAG epitope or other purification tag can facilitate purification of the protein provided herein. In some embodiments, the His tag has the sequence, (His) n, wherein n is 2, 3, 4, 5, 6, 7, 8, 9, 10, 11, 12, 13, 14, 15, 16, 17, 18, 19, 20 or greater. In one embodiment, the His tag has the sequence (His) n, wherein n is 6 (SEQ ID NO: 72).

In another embodiment, provided herein is a transgene comprising a nucleotide sequence encoding a protein comprising (or consisting of) a SARS-CoV-2 Omicron variant spike protein receptor binding domain with 1, 2, 3, 4, 5, 6, 7, 8, 9, 10, 11, or 12 amino acids deleted from the C-terminus of the receptor binding domain. In another embodiment, provided herein is a transgene comprising a nucleotide sequence encoding a protein comprising (or consisting of) a SARS-CoV-2 Omicron variant spike protein receptor binding domain with 1, 2, 3, 4, 5, 6, 7, 8, 9, 10, 11, or 12 amino acids deleted from the N-terminus of the receptor binding domain. In another embodiment, provided herein is a transgene comprising a nucleotide sequence encoding a protein comprising (or consisting of) a SARS-CoV-2 Omicron variant spike protein receptor binding domain with 1, 2, 3, 4, 5, 6, 7, 8, 9, 10, 11, or 12 amino acids deleted from the N-terminus of the receptor binding domain and 1, 2, 3, 4, 5, 6, 7, 8, 9, 10, 11, or 12 amino acids deleted from the C-terminus of the receptor binding domain. In a specific embodiment, the N-terminus is the first 25 amino acid residues of the receptor binding domain of the SARS-CoV-2 Omicron variant spike protein. In a specific embodiment, the C-terminus is the last 25 amino acid residues of the receptor binding domain of the SARS-CoV-2 Omicron variant spike protein. In certain embodiments, the protein further comprises one or more polypeptide domains. The one or more polypeptide domains may be at the C-terminus or N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His (SEQ ID NO:72)), FLAG epitope or other purification tag can facilitate purification of the protein provided herein. In some embodiments, the His tag has the sequence, (His) n, wherein n is 2, 3, 4, 5, 6, 7, 8, 9, 10, 11, 12, 13, 14, 15, 16, 17, 18, 19, 20 or greater. In one embodiment, the His tag has the sequence (His) n, wherein n is 6 (SEQ ID NO:72).

In another embodiment, described herein is a transgene comprising a nucleotide sequence encoding a protein comprising (or consisting of) the ectodomain of a SARS-CoV-2 Omicron variant spike protein. In another embodiment, described herein is a transgene comprising a nucleotide sequence encoding a protein comprising (or consisting of) a derivative of the ectodomain of a SARS-CoV-2 Omicron variant spike protein. In some embodiments, described herein is a transgene comprising a nucleotide sequence encoding a protein comprising (or consisting of) a derivative of the ectodomain of a SARS-CoV-2 Omicron variant spike protein, wherein the derivative lacks the polybasic cleavage site of the ectodomain (e.g., one, two or more residues corresponding to amino acid residues 682 to 685 (RRAR) of the spike protein of GenBank Accession No. MN908947.3 are substituted for other amino acid residues). In a specific embodiment, amino acid residues corresponding to amino acid residues 682 to 685 (RRAR) of the spike protein of GenBank Accession No. MN908947.3 are substituted with a single alanine. In another embodiment, described herein is a transgene comprising a nucleotide sequence encoding a protein comprising (or consisting of) the ectodomain of a SARS-CoV-2 Omicron variant spike protein with amino acid substitutions to proline at amino acid residues corresponding to amino acid residues 817, 892, 899, 942, 986, and 987 of the spike protein of GenBank Accession No. MN908947.3. In another embodiment, described herein is a transgene comprising a nucleotide sequence encoding a protein comprising (or consisting of) the ectodomain of a SARS-CoV-2 Omicron variant spike protein with amino acid residues corresponding to amino acid residues 682 to 685 (RRAR) of the spike protein of GenBank Accession No. MN908947.3 are substituted with a single alanine, and amino acid substitutions to proline at amino acid residues corresponding to amino acid residues 817, 892, 899, 942, 986, and 987 of the spike protein of GenBank Accession No. MN908947.3. In certain embodiments, protein further comprises one or more polypeptide domains. Useful polypeptide domains include domains that facilitate purification, folding and cleavage of portions of a polypeptide. For example, a His tag (His-His-His-His-His-His (SEQ ID NO:72)), FLAG epitope or other purification tag can facilitate purification of the protein provided herein. In some embodiments, the His tag has the sequence, (His) n, wherein n is 2, 3, 4, 5, 6, 7, 8, 9, 10, 11, 12, 13, 14, 15, 16, 17, 18, 19, 20 or greater. In a specific embodiment, a protein comprises or consists of the ectodomain of a SARS-CoV-2 Omicron variant spike protein and a His tag (e.g., a (His) n, where n is 6 (SEQ ID NO:72)). In some embodiments, a protein comprises or consists of a derivative of the ectodomain of a SARS-CoV-2 Omicron variant spike protein and a His tag (e.g., a (His) n, where n is 6 (SEQ ID NO:72)). In certain embodiments, a protein comprising (or consisting) of the ectodomain of a SARS-CoV-2 Omicron variant spike protein or a derivative thereof is a secreted polypeptide. In certain embodiments, a protein comprises the ectodomain of a SARS-CoV-2 Omicron variant spike protein or a derivative thereof comprises one or more trimerization domains known to one of skill in the art (e.g., a T4 foldon trimerization domain), and optionally a tag (e.g., a His tag or Flag tag). In a specific embodiment, when designing a protein comprising SARS-CoV-2 Omicron variant spike protein ectodomain or a derivative thereof, care is taken to maintain the stability of the resulting protein.

In some embodiments, described herein is a transgene comprising a polynucleotide sequence encoding a derivative of the ectodomain of a SARS-CoV-2 Omicron variant spike protein, wherein the polynucleotide sequence comprises a nucleotide sequence at least 80%, at least 85%, or at least 90% identical to the nucleotide sequence of SEQ ID NO: 18, 20, 22, 32, 34, 38, 40, 44, 46, 50, 52, 56, 58, 62, 64, 68, 70, 76, 78, 82, 84, 88, 90, 94, 96, 100, or 102. In some embodiments, described herein is a transgene comprising a polynucleotide sequence encoding a derivative of the ectodomain of a SARS-CoV-2 Omicron variant spike protein, wherein the polynucleotide sequence comprises a nucleotide sequence at least 95%, at least 98%, or at least 99% identical to the nucleotide sequence of SEQ ID NO: 18, 20, 22, 32, 34, 38, 40, 44, 46, 50, 52, 56, 58, 62, 64, 68, 70, 76, 78, 82, 84, 88, 90, 94, 96, 100, or 102. In some embodiments, described herein is a transgene comprising a polynucleotide sequence encoding a derivative of the ectodomain of a SARS-CoV-2 Omicron variant spike protein, wherein the polynucleotide sequence comprises the nucleotide sequence of SEQ ID NO: 18, 20, 22, 32, 34, 38, 40, 44, 46, 50, 52, 56, 58, 62, 64, 68, 70, 76, 78, 82, 84, 88, 90, 94, 96, 100, or 102.

In some embodiments, described herein is a transgene comprising a nucleotide sequence encoding a protein comprising (or consisting of) a derivative of the ectodomain of a SARS-CoV-2 Omicron variant spike protein, wherein amino acid residues corresponding to amino acid residues 682 to 685 (RRAR) of the spike protein of GenBank Accession No. MN908947.3 are substituted with a single alanine. In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with amino acid residues corresponding to amino acid residues 682 to 685 (RRAR) of the spike protein of GenBank Accession No. MN908947.3 substituted with a single alanine, and amino acid residues corresponding to 1, 2, 3, 4, 5, 6, 7, 8, 9, 10, 11, 12, 13, 14, 15, 16, 17, 18, 19, 20, or 21 of the following positions of the spike protein of GenBank Accession No. MN908947.3 substituted: A67V, T95I, G142D, L212I, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F. In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with amino acid residues corresponding to amino acid residues 682 to 685 (RRAR) of the spike protein of GenBank Accession No. MN908947.3 substituted with a single alanine, and amino acid residues corresponding to 1, 2, 3, 4, 5, 6, 7, 8, 9, 10, 11, 12, 13, 14, 15, 16, 17, 18, 19, 20, or 21 of the following positions of the spike protein of GenBank Accession No. MN908947.3 substituted: A67V, T95I, G142D, L212I, ins214EPE, G339D, S371L, S373P, S375F, K417N, N440K, G446S, S477N, T478K, E484A, Q493R, G496S, Q498R, N501Y, Y505H, T547K, D614G, H655, V687I, N764K, D796Y, N856K, Q954H, N969K, and L981F. In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with amino acid residues corresponding to amino acid residues 682 to 685 (RRAR) of the spike protein of GenBank Accession No. MN908947.3 substituted with a single alanine, and amino acid residues corresponding to 1, 2, 3, 4, 5, 6, 7, 8, 9, 10, 11, 12, 13, 14, 15, 16, 17, 18, 19, 20, or 21 of the following positions of the spike protein of GenBank Accession No. MN908947.3 substituted: L212I, G339D, S371L, S373P, S375F, K417N, N440K, G446S, S477N, T478K, E484A, Q493, G496S, Q498, N501Y, Y505H, T547K, D614G, H655Y, N679K, P681H, N764K, D796Y, N856K, Q954H, N969K, and L981F.

In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with amino acid residues corresponding to amino acid residues 682 to 685 (RRAR) of the spike protein of GenBank Accession No. MN908947.3 substituted with a single alanine, and amino acid residues corresponding to 1, 2, 3, 4, 5, 6, 7, 8, 9, 10, 11, 12, 13, 14, 15, 16, 17, 18, 19, 20, 21, or more of the following positions of the spike protein of GenBank Accession No. MN908947.3 mutated as indicated: A67V, HV69-70 deletion, T95I, G142D, VYY143-145 deletion, N211 deletion, L212I, ins214EPE, G339D, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F. In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with amino acid residues corresponding to amino acid residues 682 to 685 (RRAR) of the spike protein of GenBank Accession No. MN908947.3 substituted with a single alanine, and amino acid residues corresponding to the following positions of the spike protein of GenBank Accession No. MN908947.3 mutated as indicated: A67V, HV69-70 deletion, T95I, G142D, VYY143-145 deletion, N211 deletion, L212I, ins214EPE, G339D, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F.

In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with amino acid residues corresponding to amino acid residues 682 to 685 (RRAR) of the spike protein of GenBank Accession No. MN908947.3 substituted with a single alanine, and amino acid residues corresponding to 1, 2, 3, 4, 5, 6, 7, 8, 9, 10, 11, 12, 13, 14, 15, 16, 17, 18, 19, 20, 21, or more of the following positions of the spike protein of GenBank Accession No. MN908947.3 mutated as indicated: T19I, del24-26 (LPP), A27S, G142D, V213G, G339D, T376A, D405N, R408S, K417N, N440K, S477N, T478K, E484A, Q493R, Q498R, N501Y, Y505H, D614G, H655Y, N679K, P681H, N764K, D796Y, Q954H, and N969K. In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with amino acid residues corresponding to amino acid residues 682 to 685 (RRAR) of the spike protein of GenBank Accession No. MN908947.3 substituted with a single alanine, and amino acid residues corresponding to the following positions of the spike protein of GenBank Accession No. MN908947.3 mutated as indicated: T19I, del24-26 (LPP), A27S, G142D, V213G, G339D, T376A, D405N, R408S, K417N, N440K, S477N, T478K, E484A, Q493R, Q498R, N501Y, Y505H, D614G, H655Y, N679K, P681H, N764K, D796Y, Q954H, and N969K.

In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with amino acid residues corresponding to amino acid residues 682 to 685 (RRAR) of the spike protein of GenBank Accession No. MN908947.3 substituted with a single alanine, and amino acid residues corresponding to 1, 2, 3, 4, 5, 6, 7, 8, 9, 10, 11, 12, 13, 14, 15, 16, 17, 18, 19, 20, 21, or more of the following positions of the spike protein of GenBank Accession No. MN908947.3 mutated as indicated: T19I, del24-26 (LPP), A27S, del69-70 (HV), G142D, V213G, G339D, T376A, D405N, R408S, K417N, N440K, S477N, L452R, T478K, E484A, F486V, Q498R, N501Y, Y505H, D614G, H655Y, N679K, P681H, N764K, D796Y, Q954H, N969K. In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with amino acid residues corresponding to amino acid residues 682 to 685 (RRAR) of the spike protein of GenBank Accession No. MN908947.3 substituted with a single alanine, and amino acid residues corresponding to the following positions of the spike protein of GenBank Accession No. MN908947.3 mutated as indicated: T19I, del24-26 (LPP), A27S, del69-70 (HV), G142D, V213G, G339D, T376A, D405N, R408S, K417N, N440K, S477N, L452R, T478K, E484A, F486V, Q498R, N501Y, Y505H, D614G, H655Y, N679K, P681H, N764K, D796Y, Q954H, N969K.

In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with amino acid residues corresponding to amino acid residues 682 to 685 (RRAR) of the spike protein of GenBank Accession No. MN908947.3 substituted with a single alanine, the amino acid residues at amino acid positions corresponding to amino acid positions 817, 892, 899, 942, 986, and 987 of the spike protein found at the spike protein of GenBank Accession No. MN908947.3 substituted with prolines, and amino acid residues corresponding to 1, 2, 3, 4, 5, 6, 7, 8, 9, 10, 11, 12, 13, 14, 15, 16, 17, 18, 19, 20, 21, or more of the positions of the spike protein of GenBank Accession No. MN908947.3 mutated as indicated for one of the constructs in Table 6, 7, 8, 9, 10, or 11. In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with amino acid residues corresponding to amino acid residues 682 to 685 (RRAR) of the spike protein of GenBank Accession No. MN908947.3 substituted with a single alanine, the amino acid residues at amino acid positions corresponding to amino acid positions 817, 892, 899, 942, 986, and 987 of the spike protein found at the spike protein of GenBank Accession No. MN908947.3 substituted with prolines, and amino acid residues corresponding to the positions of the spike protein of GenBank Accession No. MN908947.3 mutated as indicated for one of the constructs in Table 6, 7, 8, 9, 10, or 11. In some embodiments, the derivative of the ectodomain comprises two or more (e.g., 3, 4, 5, 6, or 7), or all of the following amino acid modifications at amino acid positions corresponding to the indicated amino acid positions of SEQ ID NO: 104: N440K, S477N, Y505H, N679K, N764K, D796Y, Q954H, and/or N969K. In some embodiments, the derivative of the ectodomain includes a serine at amino acid positions corresponding to amino acid positions 371, 373, or 375 of SEQ ID NO: 104. In some embodiments, the derivative of the ectodomain includes a serine at amino acid positions corresponding to amino acid positions 371 or 373 of SEQ ID NO: 104. In some embodiments, the derivative of the ectodomain includes a serine at amino acid positions corresponding to amino acid positions 371, 373, and 375 of SEQ ID NO:104. In some embodiments, the derivative of the ectodomain includes a serine at amino acid positions corresponding to amino acid positions 371, 373, or 375 of SEQ ID NO: 104, and a leucine at the amino acid position corresponding to 452 of SEQ ID NO: 104. In some embodiments, the derivative of the ectodomain includes a serine at amino acid positions corresponding to amino acid positions 371 or 375 of SEQ ID NO: 104, and a leucine at the amino acid position corresponding to 452 of SEQ ID NO: 104. In some embodiments, the derivative of the ectodomain includes a serine at amino acid positions corresponding to amino acid positions 371, 373, and 375 of SEQ ID NO: 104, and a leucine at the amino acid position corresponding to 452 of SEQ ID NO: 104.

In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with amino acid residues corresponding to amino acid residues 682 to 685 (RRAR) of the spike protein of GenBank Accession No. MN908947.3 substituted with a single alanine, the amino acid residues at amino acid positions corresponding to amino acid positions 817, 892, 899, 942, 986, and 987 of the spike protein found at the spike protein of GenBank Accession No. MN908947.3 substituted with prolines and amino acid residues corresponding to 1, 2, 3, 4, 5, 6, 7, 8, 9, 10, 11, 12, 13, 14, 15, 16, 17, 18, 19, 20, 21, or more, or all of the amino acid positions of the spike protein of GenBank Accession No. MN908947.3 mutated as indicated for the construct as identified as NDV-HXP-S Omicron BA. 1 in Table 6.

In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with amino acid residues corresponding to amino acid residues 682 to 685 (RRAR) of the spike protein of GenBank Accession No. MN908947.3 substituted with a single alanine, the amino acid residues at amino acid positions corresponding to amino acid positions 817, 892, 899, 942, 986, and 987 of the spike protein found at the spike protein of GenBank Accession No. MN908947.3 substituted with prolines, and amino acid residues corresponding to 1, 2, 3, 4, 5, 6, 7, 8, 9, 10, 11, 12, 13, 14, 15, 16, 17, 18, 19, 20, 21, or more, or all of the amino acid positions of the spike protein of GenBank Accession No. MN908947.3 mutated as indicated for the construct as identified as NDV-HXP-S Omicron BA.2 (S371, S373, S375) in Table 8.

In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with amino acid residues corresponding to amino acid residues 682 to 685 (RRAR) of the spike protein of GenBank Accession No. MN908947.3 substituted with a single alanine, the amino acid residues at amino acid positions corresponding to amino acid positions 817, 892, 899, 942, 986, and 987 of the spike protein found at the spike protein of GenBank Accession No. MN908947.3 substituted with prolines, and amino acid residues corresponding to 1, 2, 3, 4, 5, 6, 7, 8, 9, 10, 11, 12, 13, 14, 15, 16, 17, 18, 19, 20, 21, or more, or all of the amino acid positions of the spike protein of GenBank Accession No. MN908947.3 mutated as indicated for the construct as identified as NDV-HXP-S Omicron BA.5 SSS L452 in Table 9.

In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with amino acid residues corresponding to amino acid residues 682 to 685 (RRAR) of the spike protein of GenBank Accession No. MN908947.3 substituted with a single alanine, the amino acid residues at amino acid positions corresponding to amino acid positions 817, 892, 899, 942, 986, and 987 of the spike protein found at the spike protein of GenBank Accession No. MN908947.3 substituted with prolines, and amino acid residues corresponding to 1, 2, 3, 4, 5, 6, 7, 8, 9, 10, 11, 12, 13, 14, 15, 16, 17, 18, 19, 20, 21, or more, or all of the amino acid positions of the spike protein of GenBank Accession No. MN908947.3 mutated as indicated for the construct as identified as NDV-HXP-S Omicron Q1.1 in Table 10.

In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with amino acid residues corresponding to amino acid residues 682 to 685 (RRAR) of the spike protein of GenBank Accession No. MN908947.3 substituted with a single alanine, the amino acid residues at amino acid positions corresponding to amino acid positions 817, 892, 899, 942, 986, and 987 of the spike protein found at the spike protein of GenBank Accession No. MN908947.3 substituted with prolines, and amino acid residues corresponding to 1, 2, 3, 4, 5, 6, 7, 8, 9, 10, 11, 12, 13, 14, 15, 16, 17, 18, 19, 20, 21, or more, or all of the amino acid positions of the spike protein of GenBank Accession No. MN908947.3 mutated as indicated for the construct as identified as NDV-HXP-S Omicron XBB.15 in Table 10.

In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with amino acid residues corresponding to amino acid residues 682 to 685 (RRAR) of the spike protein of GenBank Accession No. MN908947.3 substituted with a single alanine, the amino acid residues at amino acid positions corresponding to amino acid positions 817, 892, 899, 942, 986, and 987 of the spike protein found at the spike protein of GenBank Accession No. MN908947.3 substituted with prolines, and amino acid residues corresponding to 1, 2, 3, 4, 5, 6, 7, 8, 9, 10, 11, 12, 13, 14, 15, 16, 17, 18, 19, 20, 21, or more, or all of the amino acid positions of the spike protein of GenBank Accession No. MN908947.3 mutated as indicated for the construct as identified as NDV-HXP-S Omicron BA.1 (S371, S375) in Table 11.

In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with amino acid residues corresponding to amino acid residues 682 to 685 (RRAR) of the spike protein of GenBank Accession No. MN908947.3 substituted with a single alanine, the amino acid residues at amino acid positions corresponding to amino acid positions 817, 892, 899, 942, 986, and 987 of the spike protein found at the spike protein of GenBank Accession No. MN908947.3 substituted with prolines, and amino acid residues corresponding to amino acid positions of the spike protein of GenBank Accession No. MN908947.3 mutated as indicated for the construct identified as NDV-HXP-S Omicron BA. 1 in Table 6, NDV-HXP-S Omicron BA.2 (S371, S373, S375) in Table 8.

In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with amino acid residues corresponding to amino acid residues 682 to 685 (RRAR) of the spike protein of GenBank Accession No. MN908947.3 substituted with a single alanine, the amino acid residues at amino acid positions corresponding to amino acid positions 817, 892, 899, 942, 986, and 987 of the spike protein found at the spike protein of GenBank Accession No. MN908947.3 substituted with prolines, and amino acid residues corresponding to amino acid positions of the spike protein of GenBank Accession No. MN908947.3 mutated as indicated for the construct identified as NDV-HXP-S Omicron BA.5 SSS L452 in Table 9.

In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with amino acid residues corresponding to amino acid residues 682 to 685 (RRAR) of the spike protein of GenBank Accession No. MN908947.3 substituted with a single alanine, the amino acid residues at amino acid positions corresponding to amino acid positions 817, 892, 899, 942, 986, and 987 of the spike protein found at the spike protein of GenBank Accession No. MN908947.3 substituted with prolines, and amino acid residues corresponding to amino acid positions of the spike protein of GenBank Accession No. MN908947.3 mutated as indicated for the construct identified as NDV-HXP-S Omicron Q1.1 in Table 10.

In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with amino acid residues corresponding to amino acid residues 682 to 685 (RRAR) of the spike protein of GenBank Accession No. MN908947.3 substituted with a single alanine, the amino acid residues at amino acid positions corresponding to amino acid positions 817, 892, 899, 942, 986, and 987 of the spike protein found at the spike protein of GenBank Accession No. MN908947.3 substituted with prolines, and amino acid residues corresponding to amino acid positions of the spike protein of GenBank Accession No. MN908947.3 mutated as indicated for the construct identified as NDV-HXP-S Omicron XBB.15 in Table 10.

In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with amino acid residues corresponding to amino acid residues 682 to 685 (RRAR) of the spike protein of GenBank Accession No. MN908947.3 substituted with a single alanine, and amino acid residues corresponding to amino acid positions to the spike protein of GenBank Accession No. MN908947.3 mutated as indicated for the construct identified as NDV-HXP-S Omicron BA.1 (S371, S375) in Table 11.

In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P, A942P, K986P, and V987P; and (3) the following mutations at amino acid residues corresponding to the following amino acid residues of the spike protein of GenBank Accession No. MN908947.3: A67V, HV69-70 deletion, T95I, G142D, VYY143-145 deletion, N211 deletion, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F. In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P, A942P, K986P, and V987P; and (3) the following mutations at amino acid residues corresponding to the following amino acid residues of the spike protein of GenBank Accession No. MN908947.3: A67V, HV69-70 deletion, T95I, G142D, VYY143-145 deletion, N211 deletion, L212I, ins214EPE, G339D, S371L, S373P, S375F, K417N, N440K, G446S, S477N, T478K, E484A, Q493R, G496S, Q498R, N501Y, Y505H, T547K, D614G, H655, NSPRRARS 679-686 deletion, V687I, N764K, D796Y, N856K, Q954H, N969K, and L981F. In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P, A942P, K986P, and V987P; and (3) the following mutations at amino acid residues corresponding to the following amino acid residues of the spike protein of GenBank Accession No. MN908947.3: A67V, HV69-70 deletion, T95I, G142D, VYY143-145 deletion, N211 deletion, L212I, ins214EPE, G339D, S371L, S373P, S375F, K417N, N440K, G446S, S477N, T478K, E484A, Q493, G496S, Q498, N501Y, Y505H, T547K, D614G, H655Y, N679K, P681H, N764K, D796Y, N856K, Q954H, N969K, and L981F. In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P, A942P, K986P, and V987P; and (3) amino acid mutations at amino acid residues corresponding to 1, 2, 3, 4, 5, 6, 7, 8, or more of the amino acid residues of the spike protein of GenBank Accession No. MN908947.3 set forth in Table 5.

In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P, A942P, K986P, and V987P; and (3) amino acid mutations at amino acid residues corresponding to 1, 2, 3, 4, 5, 6, 7, 8, or more of the amino acid residues of the spike protein of GenBank Accession No. MN908947.3 set forth in Table 6. In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P, A942P, K986P, and V987P; and (3) amino acid mutations at amino acid residues corresponding to 1, 2, 3, 4, 5, 6, 7, 8, or more of the amino acid residues of the spike protein of GenBank Accession No. MN908947.3 set forth in Table 7. In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of GenBank Accession No. MN908947.3: F817P, A892P, A899P, A942P, K986P, and V987P; and (3) amino acid mutations at amino acid residues corresponding to 1, 2, 3, 4, 5, 6, 7, 8, or more of the amino acid residues of the spike protein of GenBank Accession No. MN908947.3 set forth in Table 8.

In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of GenBank Accession No. MN908947.3: F817P, A892P, A899P, A942P, K986P, and V987P; and (3) amino acid mutations at amino acid residues corresponding to 1, 2, 3, 4, 5, 6, 7, 8, or more of the amino acid residues of the spike protein of GenBank Accession No. MN908947.3 set forth in Table 9. In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of GenBank Accession No. MN908947.3: F817P, A892P, A899P, A942P, K986P, and V987P; and (3) amino acid mutations at amino acid residues corresponding to 1, 2, 3, 4, 5, 6, 7, 8, or more of the amino acid residues of the spike protein of GenBank Accession No. MN908947.3 set forth in Table 10. In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of GenBank Accession No. MN908947.3: F817P, A892P, A899P, A942P, K986P, and V987P; and (3) amino acid mutations at amino acid residues corresponding to 1, 2, 3, 4, 5, 6, 7, 8, or more of the amino acid residues of the spike protein of GenBank Accession No. MN908947.3 set forth in Table 11.

In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P, A942P, K986P, and V987P; and (3) the following mutations at amino acid residues corresponding to the following amino acid residues of the spike protein of GenBank Accession No. MN908947.3: A67V, HV69-70 deletion, T95I, G142D, VYY143-145 deletion, N211 deletion, L212I, ins214EPE, G339D, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F. In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P, A942P, K986P, and V987P; (3) the following mutations at amino acid residues corresponding to the following amino acid residues of the spike protein of GenBank Accession No. MN908947.3: A67V, HV69-70 deletion, T95I, G142D, VYY143-145 deletion, N211 deletion, L212I, ins214EPE, G339D, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F; and (4) one or two of the following amino acid substitutions at amino acid residues corresponding to the following amino acid residues of the spike protein of GenBank Accession No. MN908947.3: S371L, S373P, and S375F. In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P, A942P, K986P, and V987P; (3) the following mutations at amino acid residues corresponding to the following amino acid residues of the spike protein of GenBank Accession No. MN908947.3: A67V, HV69-70 deletion, T95I, G142D, VYY143-145 deletion, N211 deletion, L212I, ins214EPE, G339D, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F; and (4) one, two, or three amino acid substitutions at amino acid residues corresponding to the following amino acid residues of the spike protein of GenBank Accession No. MN908947.3: S371, S373, and S375, wherein the amino acid substitutions are not S371L, S373P, and S375F.

In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P, A942P, K986P, and V987P; and (3) the following mutations at amino acid residues corresponding to the following amino acid residues of the spike protein of GenBank Accession No. MN908947.3: T19I, del24-26 (LPP), A27S, G142D, V213G, G339D, T376A, D405N, R408S, K417N, N440K, S477N, T478K, E484A, Q493R, Q498R, N501Y, Y505H, D614G, H655Y, N679K, P681H, N764K, D796Y, Q954H, and N969K. In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P. A942P, K986P, and V987P; (3) the following mutations at amino acid residues corresponding to the following amino acid residues of the spike protein of GenBank Accession No. MN908947.3: T19I, del24-26 (LPP), A27S, G142D, V213G, G339D, T376A, D405N, R408S, K417N, N440K, S477N, T478K, E484A, Q493R, Q498R, N501Y, Y505H, D614G, H655Y, N679K, P681H, N764K, D796Y, Q954H, and N969K; and (4) one or two of the following amino acid substitutions at amino acid residues corresponding to the following amino acid residues of the spike protein of GenBank Accession No. MN908947.3: S371F, S373P, and S375F. In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P, A942P, K986P, and V987P; (3) the following mutations at amino acid residues corresponding to the following amino acid residues of the spike protein of GenBank Accession No. MN908947.3: T19I, del24-26 (LPP), A27S, G142D, V213G, G339D, T376A, D405N, R408S, K417N, N440K, S477N, T478K, E484A, Q493R, Q498R, N501Y, Y505H, D614G, H655Y, N679K, P681H, N764K, D796Y, Q954H, and N969K; and (4) one, two, or three amino acid substitutions at amino acid residues corresponding to the following amino acid residues of the spike protein of GenBank Accession No. MN908947.3: S371, S373, and S375, wherein the amino acid substitutions are not S371F, S373P, and S375F.

In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P, A942P, K986P, and V987P; and (3) the following mutations at amino acid residues corresponding to the following amino acid residues of the spike protein of GenBank Accession No. MN908947.3: T19I, del24-26 (LPP), A27S, del69-70 (HV), G142D, V213G, G339D, T376A, D405N, R408S, K417N, N440K, S477N, L452R, T478K, E484A, F486V, Q498R, N501Y, Y505H, D614G, H655Y, N679K, P681H, N764K, D796Y, Q954H, and N969K. In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P, A942P, K986P, and V987P; (3) the following mutations at amino acid residues corresponding to the following amino acid residues of the spike protein of GenBank Accession No. MN908947.3: T19I, del24-26 (LPP), A27S, del69-70 (HV), G142D, V213G, G339D, T376A, D405N, R408S, K417N, N440K, S477N, L452R, T478K, E484A, F486V, Q498R, N501Y, Y505H, D614G, H655Y, N679K, P681H, N764K, D796Y, Q954H, and N969K; and (4) one or two of the following amino acid substitutions at amino acid residues corresponding to the following amino acid residues of the spike protein of GenBank Accession No. MN908947.3: S371F, S373P, S375F. In some embodiments, described herein is a transgene comprising a nucleotide sequence encoding a protein comprising (or consisting of) a derivative of the ectodomain of a SARS-CoV-2 Omicron variant spike protein, wherein the derivative comprises the amino acid sequence of the spike protein of GenBank Accession No. MN908947.3 with (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P, A942P, K986P, and V987P; (3) the following mutations at amino acid residues corresponding to the following amino acid residues of the spike protein of GenBank Accession No. MN908947.3: T19I, del24-26 (LPP), A27S, del69-70 (HV), G142D, V213G, G339D, T376A, D405N, R408S, K417N, N440K, S477N, L452R, T478K, E484A, F486V, Q498R, N501Y, Y505H, D614G, H655Y, N679K, P681H, N764K, D796Y, Q954H, and N969K; and (4) one, two, or three amino acid substitutions at amino acid residues corresponding to the following amino acid residues of the spike protein of GenBank Accession No. MN908947.3: S371, S373, and S375, wherein the amino acid substitutions are not S371F, S373P, and S375F.

In another embodiment, provided herein is a transgene comprising a nucleotide sequence encoding a protein comprising (or consisting of) a SARS-CoV-2 Omicron variant spike protein ectodomain with 1, 2, 3, 4, 5, 6, 7, 8, 9, 10, 11, or 12 amino acids substituted with another amino acid (e.g., a conservative amino acid substitution). In another embodiment, provided herein is a transgene comprising a nucleotide sequence encoding a protein comprising (or consisting of) a SARS-CoV-2 Omicron variant spike protein ectodomain with 1, 2, 3, 4, 5, 6, 7, 8, 9, 10, 11, or 12 amino acids at the C-terminus of the ectodomain substituted with another amino acid (e.g., a conservative amino acid substitution). In another embodiment, provided herein is a transgene comprising a nucleotide sequence encoding a protein comprising (or consisting of) a SARS-CoV-2 Omicron variant spike protein ectodomain with 1, 2, 3, 4, 5, 6, 7, 8, 9, 10, 11, or 12 amino acids at the N-terminus of the ectodomain substituted with another amino acid (e.g., a conservative amino acid substitution). In another embodiment, provided herein is a transgene comprising a nucleotide sequence encoding a protein comprising (or consisting of) a SARS-CoV-2 Omicron variant spike protein ectodomain with 1, 2, 3, 4, 5, 6, 7, 8, 9, 10, 11, or 12 amino acids at the N-terminus of the ectodomain substituted with another amino acid (e.g., a conservative amino acid substitution) and 1, 2, 3, 4, 5, 6, 7, 8, 9, 10, 11, or 12 amino acids at the C-terminus of the ectodomain substituted with another amino acid (e.g., a conservative amino acid substitution). In some embodiments, the SARS-CoV-2 Omicron variant spike protein ectodomain is at least 90%, at least 95%, at least 96%, at least 97%, at least 98%, or at least 99% identical to the amino acid sequence of SEQ ID NO: 19, 21, 23, 33, 35, 39, 41, 45, 47, 51, 53, 57, 59, 63, 65, 69, 71, 77, 79, 83, 85, 91, 95, 97, 101, or 103. In a specific embodiment, the C-terminus of the ectodomain is the last 100 amino acid residues. In a specific embodiment, the N-terminus of the ectodomain is the first 100 amino acid residues. In certain embodiments, the protein further comprises one or more polypeptide domains. The one or more polypeptide domains may be at the C-terminus, N-terminus, or C-terminus and N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His (SEQ ID NO:72)), FLAG epitope or other purification tag can facilitate purification of the protein provided herein. In some embodiments, the His tag has the sequence, (His) n, wherein n is 2, 3, 4, 5, 6, 7, 8, 9, 10, 11, 12, 13, 14, 15, 16, 17, 18, 19, 20 or greater. In one embodiment, the His tag has the sequence (His) n, wherein n is 6 (SEQ ID NO:72). In certain embodiments, a protein comprising (or consisting) of the ectodomain of a SARS-CoV-2 Omicron variant spike protein is a secreted polypeptide. In certain embodiments, a protein comprises the ectodomain of a SARS-CoV-2 Omicron variant spike polypeptide comprises one or more trimerization domains known to one of skill in the art (e.g., a T4 foldon trimerization domain), and optionally a tag (e.g., a His tag or Flag tag).

In another embodiment, provided herein is a transgene comprising a nucleotide sequence encoding a protein comprising (or consisting of) an amino acid sequence at least 85%, at least 90%, at least 95%, at least 96%, at least 97%, at least 98%, at least 99%, or at least 99.5% identical to the amino acid sequence of SEQ ID NO: 19, 21, 23, 33, 35, 39, 41, 45, 47, 51, 53, 57, 59, 63, 65, 69, 71, 77, 79, 83, 85, 91, 95, 97, 101, or 103. In another embodiment, provided herein is a transgene comprising a nucleotide sequence encoding a protein comprising (or consisting of) an amino acid sequence at least 96%, at least 97%, at least 98%, at least 99%, or at least 99.5% identical to the amino acid sequence of SEQ ID NO: 19, 21, 23, 33, 35, 39, 41, 45, 47, 51, 53, 57, 59, 63, 65, 69, 71, 77, 79, 83, 85, 91, 95, 97, 101, or 103. In another embodiment, provided herein is a transgene comprising a nucleotide sequence encoding a protein comprising (or consisting of) the amino acid sequence of SEQ ID NO: 19, 21, 23, 33, 35, 39, 41, 45, 47, 51, 53, 57, 59, 63, 65, 69, 71, 77, 79, 83, 85, 91, 95, 97, 101, or 103. In certain embodiments, the protein further comprises one or more polypeptide domains. The one or more polypeptide domains may be at the C-terminus, N-terminus, or C-terminus and N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His (SEQ ID NO:72)), FLAG epitope or other purification tag can facilitate purification of the protein provided herein. In some embodiments, the His tag has the sequence, (His) n, wherein n is 2, 3, 4, 5, 6, 7, 8, 9, 10, 11, 12, 13, 14, 15, 16, 17, 18, 19, 20 or greater. In one embodiment, the His tag has the sequence (His) n, wherein n is 6 (SEQ ID NO:72). In certain embodiments, the protein is a secreted polypeptide. In some embodiments, a protein comprises further comprises NDV F protein transmembrane and cytoplasmic domains. In some embodiments, a protein comprises further comprises one or more trimerization domains known to one of skill in the art (e.g., a T4 foldon trimerization domain), and optionally a tag (e.g., a His tag or Flag tag).

In another embodiment, provided herein is a transgene comprising a nucleotide sequence encoding a protein comprising (or consisting of) a SARS-CoV-2 Omicron variant spike protein ectodomain with 1, 2, 3, 4, 5, 6, 7, 8, 9, 10, 11, or 12 amino acid substitutions and 1, 2, 3, 4, 5, 6, 7, 8, 9, 10, 11, or 12 amino acids deleted. In another embodiment, provided herein is a transgene comprising a nucleotide sequence encoding a protein comprising (or consisting of) a SARS-CoV-2 Omicron variant spike protein ectodomain with 1, 2, 3, 4, 5, 6, 7, 8, 9, 10, 11, or 12 amino acids deleted from the C-terminus. In another embodiment, provided herein is a transgene comprising a nucleotide sequence encoding a protein comprising (or consisting of) a SARS-CoV-2 Omicron variant spike protein ectodomain with 1, 2, 3, 4, 5, 6, 7, 8, 9, 10, 11, or 12 amino acids deleted from the N-terminus. In certain embodiments, the SARS-CoV-2 Omicron variant spike protein ectodomain lacks the polybasic cleavage site (e.g., amino acid residues 682 to 685 (RRAR) are substituted with a single alanine). In some embodiments, the SARS-CoV-2 Omicron variant spike protein ectodomain comprises the following amino acid substitutions at amino acid residues corresponding to at amino acid residues of the spike protein of GenBank Accession No. MN908947.3: F817P, A892P, A899P, A942P, K986P, and V987P. In a specific embodiment, the SARS-CoV-2 Omicron variant spike protein ectodomain comprises an amino acid substitution at amino acid residues corresponding to amino acid residues 682 to 685 (RRAR) of the spike protein of GenBank Accession No. MN908947.3 with a single alanine, and the following amino acid substitutions at amino acid residues corresponding to at amino acid residues of the spike protein of GenBank Accession No. MN908947.3: F817P, A892P, A899P, A942P, K986P, and V987P. In some embodiments, the SARS-CoV-2 Omicron variant spike protein ectodomain is at least 90%, at least 95%, at least 96%, at least 97%, at least 98%, or at least 99% identical to the amino acid sequence of SEQ ID NO:19, 21, 23, 33, 35, 39, 41, 45, 47, 51, 53, 57, 59, 63, 65, 69, 71, 77, 79, 83, 85, 91, 95, 97, 101, or 103. In some embodiments, the SARS-CoV-2 Omicron variant spike protein ectodomain comprises two or more (e.g., 3, 4, 5, 6, or 7), or all of the following amino acid modifications at amino acid positions corresponding to the indicated amino acid positions of SEQ ID NO: 104: N440K, S477N, Y505H, N679K, N764K, D796Y, Q954H, and/or N969K. In some embodiments, the SARS-CoV-2 spike protein ectodomain includes a serine at amino acid positions corresponding to amino acid positions 371, 373, and/or 375 of SEQ ID NO:104. In some embodiments, the SARS-CoV-2 spike protein ectodomain includes a leucine at the amino acid position corresponding to amino acid position 452 of SEQ ID NO:104. In a specific embodiment, the C-terminus of the ectodomain is the last 100 amino acid residues. In a specific embodiment, the N-terminus of the ectodomain is the first 100 amino acid residues. In certain embodiments, the protein further comprise one or more polypeptide domains. The one or more polypeptide domains may be at the C-terminus, N-terminus, or C-terminus and N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His (SEQ ID NO:72)), FLAG epitope or other purification tag can facilitate purification of the protein provided herein. In some embodiments, the His tag has the sequence, (His) n, wherein n is 2, 3, 4, 5, 6, 7, 8, 9, 10, 11, 12, 13, 14, 15, 16, 17, 18, 19, 20 or greater. In one embodiment, the His tag has the sequence (His) n, wherein n is 6 (SEQ ID NO: 72). In some embodiments, a protein comprises further comprises NDV F protein transmembrane and cytoplasmic domains. In some embodiments, a protein that comprises the ectodomain of a SARS-CoV-2 Omicron variant spike protein further comprises one or more trimerization domains known to one of skill in the art (e.g., a T4 foldon trimerization domain), and optionally a tag (e.g., a His tag or Flag tag).

In another embodiment, provided herein is a transgene comprising a nucleotide sequence encoding a protein comprising (or consisting of) a SARS-CoV-2 Omicron variant spike protein ectodomain with 1, 2, 3, 4, 5, 6, 7, 8, 9, 10, 11, or 12 amino acids deleted. In another embodiment, provided herein is a transgene comprising a nucleotide sequence encoding a protein comprising (or consisting of) a SARS-CoV-2 Omicron variant spike protein ectodomain with 1, 2, 3, 4, 5, 6, 7, 8, 9, 10, 11, or 12 amino acids deleted from the C-terminus. In another embodiment, provided herein is a transgene comprising a nucleotide sequence encoding a protein comprising (or consisting of) a SARS-CoV-2 Omicron variant spike protein ectodomain with 1, 2, 3, 4, 5, 6, 7, 8, 9, 10, 11, or 12 amino acids deleted from the N-terminus. In certain embodiments, the SARS-CoV-2 Omicron variant spike protein ectodomain lacks the polybasic cleavage site (e.g., one, two or more residues corresponding to amino acid residues 682 to 685 (RRAR) of the spike protein of GenBank Accession No. MN908947.3 are substituted for other amino acid residues). In a specific embodiment, amino acid residues corresponding to amino acid residues 682 to 685 (RRAR) of the spike protein of GenBank Accession No. MN908947.3 are substituted with a single alanine. In some embodiments, amino acid substitutions corresponding to the following amino acid residues of the spike protein of GenBank Accession No. MN908947.3 are substituted: F817P, A892P, A899P, A942P, K986P, and V987P. In some embodiments, the SARS-CoV-2 Omicron variant spike protein ectodomain is at least 90%, at least 95%, at least 96%, at least 97%, at least 98%, or at least 99% identical to SEQ ID NO: 19, 21, 23, 33, 35, 39, 41, 45, 47, 51, 53, 57, 59, 63, 65, 69, 71, 77, 79, 83, 85, 91, 95, 97, 101, or 103. In certain embodiments, the protein further comprise one or more polypeptide domains. The one or more polypeptide domains may be at the C-terminus, N-terminus, or C-terminus and N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His (SEQ ID NO:72)), FLAG epitope or other purification tag can facilitate purification of the protein provided herein. In some embodiments, the His tag has the sequence, (His) n, wherein n is 2, 3, 4, 5, 6, 7, 8, 9, 10, 11, 12, 13, 14, 15, 16, 17, 18, 19, 20 or greater. In one embodiment, the His tag has the sequence (His) n, wherein n is 6 (SEQ ID NO:72). In some embodiments, a protein comprises further comprises NDV F protein transmembrane and cytoplasmic domains. In certain embodiments, a protein that comprises the ectodomain of a SARS-CoV-2 Omicron variant spike protein comprises one or more trimerization domains known to one of skill in the art (e.g., a T4 foldon trimerization domain), and optionally a tag (e.g., a His tag or Flag tag).

In another embodiment, provided herein is a transgene comprising a nucleotide sequence encoding a protein comprising (or consisting of) a SARS-CoV-2 Omicron variant spike protein ectodomain with 1, 2, 3, 4, 5, 6, 7, 8, 9, 10, 11, 12, 13, 14, or 15 mutations (e.g., amino acid substitutions, amino acid deletions, amino acid additions, or a combination thereof). In another embodiment, provided herein is a transgene comprising a nucleotide sequence encoding a protein comprising (or consisting of) a SARS-CoV-2 Omicron variant spike protein ectodomain with 1, 2, 3, 4, 5, 6, 7, 8, 9, 10, 11, or 12 amino acid substitutions and 1, 2, 3, 4, 5, 6, 7, 8, 9, 10, 11, or 12 amino acids deleted. In certain embodiments, the SARS-CoV-2 Omicron variant spike protein ectodomain lacks the polybasic cleavage site (e.g., one, two or more residues corresponding to amino acid residues 682 to 685 (RRAR) of the spike protein of GenBank Accession No. MN908947.3 are substituted for other amino acid residues). In a specific embodiment, amino acid residues corresponding to amino acid residues 682 to 685 (RRAR) of the spike protein of GenBank Accession No. MN908947.3 are substituted with a single alanine. In some embodiments, amino acid substitutions corresponding to the following amino acid residues of the spike protein of GenBank Accession No. MN908947.3 are substituted: F817P, A892P, A899P, A942P, K986P, and V987P. In some embodiments, the SARS-CoV-2 Omicron variant spike protein ectodomain is at least 90%, at least 95%, at least 96%, at least 97%, at least 98%, or at least 99% identical to the amino acid sequence of SEQ ID NO: 19, 21, 23, 33, 35, 39, 41, 45, 47, 51, 53, 57, 59, 63, 65, 69, 71, 77, 79, 83, 85, 91, 95, 97, 101, or 103. In certain embodiments, the protein further comprise one or more polypeptide domains. The one or more polypeptide domains may be at the C-terminus, N-terminus, or C-terminus and N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His (SEQ ID NO:72)), FLAG epitope or other purification tag can facilitate purification of the protein provided herein. In some embodiments, the His tag has the sequence, (His) n, wherein n is 2, 3, 4, 5, 6, 7, 8, 9, 10, 11, 12, 13, 14, 15, 16, 17, 18, 19, 20 or greater. In some embodiments, a protein comprises further comprises NDV F protein transmembrane and cytoplasmic domains. In certain embodiments, a protein that comprises the ectodomain of a SARS-CoV-2 Omicron variant spike protein comprises one or more trimerization domains known to one of skill in the art (e.g., a T4 foldon trimerization domain), and optionally a tag (e.g., a His tag or Flag tag).

In another embodiment, provided herein is a transgene comprising a nucleotide sequence encoding a protein, wherein the protein comprises a spike protein ectodomain that is at least 85%, at least 90%, at least 95%, at least 96%, at least 97%, at least 98%, at least 99%, or at least 99.5% identical to the amino acid sequence of SEQ ID NO: 19, 21, 23, 33, 35, 39, 41, 45, 47, 51, 53, 57, 59, 63, 65, 69, 71, 77, 79, 83, 85, 91, 95, 97, 101, or 103. In another embodiment, provided herein is a transgene comprising a nucleotide sequence encoding a protein, wherein the protein comprises a spike protein ectodomain that is at least 95%, at least 96%, at least 97%, at least 98%, at least 99%, or at least 99.5% identical to the amino acid sequence of SEQ ID NO: 19, 21, 23, 33, 35, 39, 41, 45, 47, 51, 53, 57, 59, 63, 65, 69, 71, 77, 79, 83, 85, 91, 95, 97, 101, or 103. In another embodiment, provided herein is a transgene comprising a nucleotide sequence encoding a protein, wherein the protein comprises a spike protein ectodomain that is at least 97%, at least 98%, at least 99%, or at least 99.5% identical to the amino acid sequence of SEQ ID NO: 19, 21, 23, 33, 35, 39, 41, 45, 47, 51, 53, 57, 59, 63, 65, 69, 71, 77, 79, 83, 85, 91, 95, 97, 101, or 103. Methods/techniques known in the art may be used to determine sequence identity (see, e.g., “Best Fit” or “Gap” program of the Sequence Analysis Software Package, version 10; Genetics Computer Group, Inc.).

In another embodiment, a SARS-CoV-2 spike protein ectodomain or a derivative thereof comprises an amino acid sequence that is at least 85%, at least 90%, at least 95%, at least 96%, at least 97%, at least 98%, at least 99%, or at least 99.5% identical to the amino acid sequence of SEQ ID NO: 19, 21, 23, 33, 35, 39, 41, 45, 47, 51, 53, 57, 59, 63, 65, 69, 71, 77, 79, 83, 85, 91, 95, 97, 101, or 103. In another embodiment, a SARS-CoV-2 spike protein ectodomain or a derivative thereof comprises an amino acid sequence that is at least 95%, at least 96%, at least 97%, at least 98%, at least 99%, or at least 99.5% identical to the amino acid sequence of SEQ ID NO: 19, 21, 23, 33, 35, 39, 41, 45, 47, 51, 53, 57, 59, 63, 65, 69, 71, 77, 79, 83, 85, 91, 95, 97, 101, or 103. In another embodiment, a SARS-CoV-2 spike protein ectodomain or a derivative thereof comprises an amino acid sequence that is at least 97%, at least 98%, at least 99%, or at least 99.5% identical to the nucleotide sequence of SEQ ID NO: 19, 21, 23, 33, 35, 39, 41, 45, 47, 51, 53, 57, 59, 63, 65, 69, 71, 77, 79, 83, 85, 91, 95, 97, 101, or 103. Methods/techniques known in the art may be used to determine sequence identity (see, e.g., “Best Fit” or “Gap” program of the Sequence Analysis Software Package, version 10; Genetics Computer Group, Inc.).

In another embodiment, described herein are transgenes comprising a nucleotide sequence encoding a chimeric F protein, wherein the chimeric F protein comprises a SARS-CoV-2 Omicron variant spike protein ectodomain described herein and NDV F protein transmembrane and cytoplasmic domains. In another embodiment, described herein are transgenes comprising a nucleotide sequence encoding a chimeric F protein, wherein the chimeric F protein comprises a derivative of SARS-CoV-2 Omicron variant spike protein ectodomain described herein and NDV F protein transmembrane and cytoplasmic domains. In specific embodiments, the entire NDV F protein transmembrane and cytoplasmic domains is included in a chimeric F protein. In a specific embodiment, the NDV F protein transmembrane and cytoplasmic domains comprise the amino acid sequence of SEQ ID NO: 5. In some embodiments, the entire NDV F protein transmembrane and cytoplasmic domains is not included in a chimeric F protein. For example, a few amino acid residues (e.g., 1, 2, 3, 4, 5, 6, 7, 8, 9, 10, 11, 12, 13, 14, 15, 1-5, 1-10, or 5-15 amino acid residues) upstream to the NDV F protein transmembrane may be included in a chimeric F protein and/or a few amino acid residues (e.g., 1-5, 1-10, or 5-15 amino acid residues) downstream of the NDV F protein cytoplasmic domain may be included in a chimeric F protein. For example, a few amino acid residues (e.g., 1, 2, 3, 4, 5, or 1-5 amino acid residues) less than the entire NDV F protein transmembrane may be included in a chimeric F protein and/or a few amino acid residues (e.g., 1, 2, 3, 4, 5, or 1-5 amino acid residues) less than the entire NDV F protein cytoplasmic domain may be included. In specific embodiments, described herein are transgenes comprising a nucleotide sequence encoding a chimeric F protein, wherein the chimeric F protein comprises a SARS-CoV-2 spike protein ectodomain described herein, a NDV F protein transmembrane domain plus or minus 1, 2, 3, 4, or 5 amino acid residues, and a NDV F protein cytoplasmic domain plus or minus 1, 2, 3, 4, or 5 amino acid residues. In specific embodiments, described herein are transgenes comprising a nucleotide sequence encoding a chimeric F protein, wherein the chimeric F protein comprises a derivative of a SARS-CoV-2 spike protein ectodomain described herein, a NDV F protein transmembrane domain plus or minus 1, 2, 3, 4, or 5 amino acid residues, and a NDV F protein cytoplasmic domain plus or minus 1, 2, 3, 4, or 5 amino acid residues. In specific embodiments, the entire transmembrane and cytoplasmic domains of the SARS-CoV-2 spike protein are not present in the chimeric F protein. In some embodiments, 1, 2, or 3 amino acid residues of the transmembrane domain and/or cytoplasmic domain of the SARS-CoV-2 spike protein are present in the chimeric F protein. The ectodomain, transmembrane and cytoplasmic domains of the SARS-CoV-2 spike protein and NDV F protein may be determined using techniques known to one of skill in the art. For example, published information, GenBank or websites such as VIPR virus pathogen website (www.viprbrc.org), DTU Bioinformatics domain website (www.cbs.dtu.dk/services/TMHMM/) or programs available to determine the transmembrane domain may be used to determine the ectodomain, transmembrane and cytoplasmic domains of the SARS-CoV-2 spike protein and NDV F protein. See, e.g., Table 2, infra, with the transmembrane and cytoplasmic domains of NDV F protein indicated. In specific embodiments, the SARS-CoV-2 spike protein ectodomain is fused to the NDV F protein transmembrane and cytoplasmic domains via a linker (e.g., GGGGS (SEQ ID NO:24)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO:73), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused directly to the SARS-CoV-2 spike protein ectodomain. In certain embodiments, the transgene encoding the chimeric F protein is codon optimized. See, e.g., Section 5.1.4, infra, for a discussion regarding codon optimization.

In specific embodiment, described herein is a transgene comprising a nucleotide sequence encoding a chimeric F protein, wherein the transgene comprises an RNA sequence corresponding to the negative sense of the cDNA sequence of SEQ ID NO:6, 7, 10, 11, 14, 15, 30, 36, 42, 48, 54, 60, 66, 74, 80, 86, 92, or 98. In specific embodiment, described herein is a transgene comprising a nucleotide sequence encoding a chimeric F protein, wherein the transgene comprises an RNA sequence corresponding to the negative sense of the cDNA sequence of SEQ ID NO: 30, 42, 54, 66, 80, 86, 92, or 98. In some embodiments, described herein is a transgene comprising a nucleotide sequence encoding a chimeric F protein, wherein the transgene comprises an RNA sequence corresponding to the negative sense of a nucleotide sequence that is at least 80%, at least 85%, at least 90%, at least 95%, at least 96%, at least 97%, at least 98%, or at least 99% identical to the cDNA sequence of SEQ ID NO: 6, 7, 10, 11, 14, 15, 30, 36, 42, 48, 54, 60, 66, 74, 80, 86, 92, or 98. In some embodiments, described herein is a transgene comprising a nucleotide sequence encoding a chimeric F protein, wherein the transgene comprises an RNA sequence corresponding to the negative sense of a nucleotide sequence that is at least 80%, at least 85%, at least 90%, at least 95%, at least 96%, at least 97%, at least 98%, or at least 99% identical to the cDNA sequence of SEQ ID NO: 30, 42, 54, 66, 80, 86, 92, or 98. In some embodiments, described herein is a transgene comprising a nucleotide sequence encoding a chimeric F protein, wherein the transgene comprises an RNA sequence corresponding to the negative sense of a nucleotide sequence that is at least 80%, at least 85%, at least 90%, at least 95%, at least 96%, at least 97%, at least 98%, or at least 99% identical to the cDNA sequence of SEQ ID NO: 30, 42, 54, 66, 80, 86, 92, or 98, without the nucleotide sequence encoding the signal peptide. In specific embodiment, described herein is a transgene comprising a nucleotide sequence encoding a chimeric F protein, wherein the transgene comprises the nucleotide sequence of SEQ ID NO:6, 7, 10, 11, 14, 15, 30, 36, 42, 48, 54, 60, 66, 74, 80, 86, 92, or 98. In some embodiments, described herein is a transgene comprising a nucleotide sequence encoding a chimeric F protein, wherein the transgene comprises a nucleotide sequence that is at least 80%, at least 85%, at least 90%, at least 95%, at least 96%, at least 97%, at least 98%, or at least 99% identical to the nucleotides sequence of SEQ ID NO:6, 7, 10, 11, 14, 15, 30, 36, 42, 48, 54, 60, 66, 74, 80, 86, 92, or 98. In some embodiments, described herein is a transgene comprising a nucleotide sequence encoding a chimeric F protein, wherein the transgene comprises a nucleotide sequence that is at least 80%, at least 85%, at least 90%, at least 95%, at least 96%, at least 97%, at least 98%, or at least 99% identical to the nucleotides sequence of SEQ ID NO: 30, 42, 54, 66, 80, 86, 92, or 98. In some embodiments, described herein is a transgene comprising a nucleotide sequence encoding a chimeric F protein, wherein the transgene comprises a nucleotide sequence that is at least 80%, at least 85%, at least 90%, at least 95%, at least 96%, at least 97%, at least 98%, or at least 99% identical to the nucleotides sequence of SEQ ID NO: 30, 42, 54, 66, 80, 86, 92, or 98, without the nucleotide sequence encoding the signal peptide. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between NDV NP and P transcription units or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another specific embodiment, described herein is a transgene comprising a nucleotide sequence encoding a chimeric F protein, wherein the chimeric F protein comprises the amino acid sequence of SEQ ID NO: 19, 21, 23, 33, 35, 39, 41, 45, 47, 51, 53, 57, 59, 63, 65, 69, 71, 77, 79, 83, 85, 89, 91, 95, 97, 101, or 103, and NDV F protein transmembrane and cytoplasmic domains. In another specific embodiment, described herein is a transgene comprising a nucleotide sequence encoding a chimeric F protein, wherein the chimeric F protein comprises the amino acid sequence of SEQ ID NO: 33, 35, 45, 47, 57, 83, 85, 89, 91, 95, 97, 101, or 103, and NDV F protein transmembrane and cytoplasmic domains. In another specific embodiment, described herein is a transgene comprising a nucleotide sequence encoding a chimeric F protein, wherein the chimeric F protein comprises the amino acid sequence of SEQ ID NO: 31, 43, 55, 81, 87, 93, or 99, without the signal peptide. In another specific embodiment, described herein is a transgene comprising a nucleotide sequence encoding a chimeric F protein, wherein the chimeric F protein comprises the amino acid sequence of SEQ ID NO: 31, 43, 55, 81, 87, 93, or 99. In certain embodiments, the transgene encoding the chimeric F protein is codon optimized. See, e.g., Section 5.1.4, infra, for a discussion regarding codon optimization. In a preferred embodiment, a transgene comprises a codon-optimized version of a nucleic acid sequence encoding the chimeric F protein. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between NDV NP and P transcription units or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In certain embodiments, a transgene comprises a codon-optimized version of a nucleic acid sequence encoding the derivative of the ectodomain of the SARS-CoV-2 spike protein. In a specific embodiment, a transgene described herein comprises a nucleotide sequence encoding the amino acid sequence set forth in SEQ ID NO:8, 9, 12, 13, 16, 17, 31, 37, 43, 49, 55, 61, 67, 81, 87, 93, or 99. In a specific embodiment, a transgene described herein comprises a nucleotide sequence encoding an amino acid sequence that is at least 90%, at least 95%, at least 96%, at least 97%, at least 98%, or at least 99% identical to the amino acid sequence set forth in SEQ ID NO:8, 9, 12, 13, 16, 17, 31, 37, 43, 49, 55, 61, 67, 81, 87, 93, or 99. In another specific embodiment, a transgene described herein comprises the nucleotide sequence of SEQ ID NO:6, 7, 10, 11, 14, 15, 30, 36, 42, 48, 54, 60, 66, 74, 80, 86, 92, or 98, or an RNA sequence corresponding to the negative sense of the cDNA sequence of SEQ ID NO: 6, 7, 10, 11, 14, 15, 30, 36, 42, 48, 54, 60, 66, 74, 80, 86, 92, or 98. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between NDV NP and P transcription units or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, described herein are transgenes comprising a nucleotide sequence encoding a chimeric F protein, wherein the chimeric F protein comprises a SARS-CoV-2 Omicron variant spike protein ectodomain plus or minus 1, 2, 3, 4, 5, 6, 7, 8 or more amino acid residues at C-terminus of the ectodomain and NDV F protein transmembrane and cytoplasmic domains. In other words, the portion of the SARS-CoV-2 Omicron variant spike protein encoded by the chimeric F protein does not include the entire SARS-CoV-2 Omicron variant spike protein transmembrane and cytoplasmic domains. The ectodomain, transmembrane and cytoplasmic domains of the SARS-CoV-2 Omicron spike protein and NDV F protein may be determined using techniques known to one of skill in the art. For example, published information, GenBank or websites such as VIPR virus pathogen website (www.viprbrc.org), DTU Bioinformatics domain website (www.cbs.dtu.dk/services/TMHMM/) or programs available to determine the transmembrane domain may be used to determine the ectodomain, transmembrane and cytoplasmic domains of the SARS-CoV-2 spike protein and NDV F protein. See, e.g., Table 2, infra, with the transmembrane and cytoplasmic domains of NDV F protein indicated (SEQ ID NO:5). In specific embodiments, the SARS-CoV-2 Omicron variant spike protein ectodomain is fused to the NDV F protein transmembrane and cytoplasmic domains via a linker (e.g., GGGGS (SEQ ID NO:24)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO:73), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused to directly to the SARS-CoV-2 Omicron variant spike protein ectodomain. In certain embodiments, the transgene encoding the chimeric F protein is codon optimized. See, e.g., Section 5.1.4, infra, for a discussion regarding codon optimization. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between NDV NP and P transcription units or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, described herein are transgenes comprising a nucleotide sequence encoding a chimeric F protein, wherein the chimeric F protein comprises a derivative of a SARS-CoV-2 Omicron variant spike protein ectodomain and NDV F protein transmembrane and cytoplasmic domains, wherein the derivative comprises plus or minus 1, 2, 3, 4, 5, 6, 7, 8 or more amino acid residues at C-terminus of the ectodomain. In other words, the portion of the SARS-CoV-2 Omicron variant spike protein encoded by the chimeric F protein does not include the entire SARS-CoV-2 spike protein transmembrane and cytoplasmic domains. In specific embodiments, the derivative of the SARS-CoV-2 Omicron variant spike protein ectodomain lacks the polybasic cleavage site (e.g., one, two or more residues corresponding to amino acid residues 682 to 685 (RRAR) of the spike protein of GenBank Accession No. MN908947.3 are substituted for other amino acid residues). In specific embodiments, the lack of a polybasic cleavage means that the polybasic site is altered such that it cannot be cleaved by, e.g., furin. In a specific embodiment, amino acid residues corresponding to amino acid residues 682 to 685 (RRAR) of the spike protein of GenBank Accession No. MN908947.3 are substituted with a single alanine. In some embodiments, the derivative comprises the following amino acid substitutions at amino acid residues corresponding to at amino acid residues of the spike protein of GenBank Accession No. MN908947.3: F817P, A892P, A899P, A942P, K986P, and V987P. In a specific embodiment, the derivative comprises an amino acid substitution at amino acid residues corresponding to amino acid residues 682 to 685 (RRAR) of the spike protein of GenBank Accession No. MN908947.3 with a single alanine, and the following amino acid substitutions at amino acid residues corresponding to at amino acid residues of GenBank Accession No. MN908947.3: F817P, A892P, A899P, A942P, K986P, and V987P. In some embodiments, the derivative of a SARS-CoV-2 Omicron variant spike protein ectodomain is at least 90%, at least 95%, at least 96%, at least 97%, at least 98%, or at least 99% identical to SEQ ID NO: 19, 21, 23, 35, 41, 47, 53, 59, 65, 71, 79, 85, 91, 97, or 103. In some embodiments, the derivative of a SARS-CoV-2 Omicron variant spike protein ectodomain is at least 90%, at least 95%, at least 96%, at least 97%, at least 98%, or at least 99% identical to SEQ ID NO: 33, 39, 45, 51, 57, 63, 69, 77, 83, 89, 95, or 101. The ectodomain, transmembrane and cytoplasmic domains of the SARS-CoV-2 Omicron variant spike protein and NDV F protein may be determined using techniques known to one of skill in the art. For example, published information, GenBank or websites such as VIPR virus pathogen website (www.viprbrc.org), DTU Bioinformatics domain website (www.cbs.dtu.dk/services/TMHMM/) or programs available to determine the transmembrane domain may be used to determine the ectodomain, transmembrane and cytoplasmic domains of the SARS-CoV-2 Omicron variant spike protein and NDV F protein. See, e.g., Table 2, infra, with the transmembrane and cytoplasmic domains of NDV F protein indicated. In specific embodiments, the derivative of the SARS-CoV-2 Omicron variant spike protein ectodomain is fused to the NDV F protein transmembrane and cytoplasmic domains via a linker (e.g., GGGGS (SEQ ID NO:24)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO: 73), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused directly to the derivative of the SARS-CoV-2 Omicron variant spike protein ectodomain. In certain embodiments, the transgene encoding the chimeric F protein is codon optimized. See, e.g., Section 5.1.4, infra, for a discussion regarding codon optimization. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between NDV NP and P transcription units or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein comprising (or consisting of) a SARS-CoV-2 Omicron variant spike protein ectodomain with 1, 2, 3, 4, 5, 6, 7, 8, 9, 10, 11, 12, 13, 14, 15, 16, 17, 18, 19, 20 or more amino acids substituted with another amino acid (e.g., a conservative amino acid substitution) and NDV F protein transmembrane and cytoplasmic domains. In another embodiment, provided herein is a transgene comprising a nucleotide sequence encoding a chimeric F protein comprising (or consisting of) a SARS-CoV-2 Omicron variant spike protein ectodomain with 1, 2, 3, 4, 5, 6, 7, 8, 9, 10, 11, or 12 amino acids at the C-terminus of the ectodomain substituted with another amino acid (e.g., a conservative amino acid substitution) and NDV F protein transmembrane and cytoplasmic domains. In another embodiment, provided herein is a transgene comprising a nucleotide sequence encoding a chimeric F protein comprising (or consisting of) a SARS-CoV-2 Omicron variant spike protein ectodomain with 1, 2, 3, 4, 5, 6, 7, 8, 9, 10, 11, or 12 amino acids at the N-terminus of the ectodomain substituted with another amino acid (e.g., a conservative amino acid substitution) and NDV F protein transmembrane and cytoplasmic domains. In another embodiment, provided herein is a transgene comprising a nucleotide sequence encoding a chimeric F protein comprising (or consisting of) a SARS-CoV-2 Omicron variant spike protein ectodomain with 1, 2, 3, 4, 5, 6, 7, 8, 9, 10, 11, or 12 amino acids at the N-terminus substituted with another amino acid (e.g., a conservative amino acid substitution) and 1, 2, 3, 4, 5, 6, 7, 8, 9, 10, 11, or 12 amino acids at the C-terminus substituted with another amino acid (e.g., a conservative amino acid substitution), and NDV F protein transmembrane and cytoplasmic domains. In a specific embodiment, the C-terminus of the ectodomain is the last 100 amino acid residues. In a specific embodiment, the N-terminus of the ectodomain is the first 100 amino acid residues. In some embodiments, the derivative of a SARS-CoV-2 Omicron variant spike protein ectodomain is at least 90%, at least 95%, at least 96%, at least 97%, at least 98%, or at least 99% identical to SEQ ID NO: 19, 21, 23, 33, 35, 39, 41, 45, 47, 51, 53, 57, 59, 63, 65, 69, or 71. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In specific embodiments, the NDV F protein transmembrane and cytoplasmic domains of the chimeric protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein, wherein the chimeric F protein comprises (or consists of) a derivative of a SARS-CoV-2 Omicron variant spike protein ectodomain with 1, 2, 3, 4, 5, 6, 7, 8, 9, 10, 11, 12, 13, 14, 15, 16, 17, 18, 19, 20, or 21 amino acids substituted with another amino acid (e.g., a conservative amino acid substitution) and NDV F protein transmembrane and cytoplasmic domains. In specific embodiments, the derivative of the SARS-CoV-2 Omicron variant spike protein ectodomain lacks the polybasic cleavage site (e.g., one, two or more residues corresponding to amino acid residues 682 to 685 (RRAR) of the spike protein of GenBank Accession No. MN908947.3 are substituted for other amino acid residues). In specific embodiments, the lack of a polybasic cleavage means that the polybasic site is altered such that it cannot be cleaved by, e.g., furin. In a specific embodiment, the derivative of the SARS-CoV-2 Omicron variant spike protein ectodomain comprises amino acid residues corresponding to amino acid residues 682 to 685 (RRAR) of the spike protein of GenBank Accession No. MN908947.3 are substituted with a single alanine. In some embodiments, the derivative comprises the following amino acid substitutions at amino acid residues corresponding to at amino acid residues of the spike protein of GenBank Accession No. MN908947.3: F817P, A892P, A899P, A942P, K986P, and V987P. In a specific embodiment, the derivative comprises an amino acid substitution at amino acid residues corresponding to amino acid residues 682 to 685 (RRAR) of the spike protein of GenBank Accession No. MN908947.3 with a single alanine, and the following amino acid substitutions at amino acid residues corresponding to at amino acid residues of the spike protein of GenBank Accession No. MN908947.3: F817P, A892P, A899P, A942P, K986P, and V987P. In specific embodiments, the derivative of the SARS-CoV-2 spike protein ectodomain is fused to the NDV F protein transmembrane and cytoplasmic domains via a linker (e.g., GGGGS (SEQ ID NO:24)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO:73), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In other embodiments, the derivative of the SARS-CoV-2 spike protein ectodomain is fused directly to the NDV F protein transmembrane and cytoplasmic domains. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In specific embodiments, the NDV F protein transmembrane and cytoplasmic domains of the chimeric protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein comprising (or consisting of) a SARS-CoV-2 Omicron variant spike protein ectodomain with 1, 2, 3, 4, 5, 6, 7, 8, 9, 10, 11, or 12 amino acids deleted, and NDV F protein transmembrane and cytoplasmic domains. In another embodiment, provided herein is a transgene comprising a nucleotide sequence encoding a chimeric F protein comprising (or consisting of) a SARS-CoV-2 Omicron variant spike protein ectodomain with 1, 2, 3, 4, 5, 6, 7, 8, 9, 10, 11, or 12 amino acids deleted from the C-terminus, and NDV F protein transmembrane and cytoplasmic domains. In another embodiment, provided herein is a transgene comprising a nucleotide sequence encoding a chimeric F protein comprising (or consisting of) a SARS-CoV-2 Omicron variant spike protein ectodomain with 1, 2, 3, 4, 5, 6, 7, 8, 9, 10, 11, or 12 amino acids deleted from the N-terminus, and NDV F protein transmembrane and cytoplasmic domains. In a specific embodiment, the C-terminus of the ectodomain is the last 100 amino acid residues. In a specific embodiment, the N-terminus of the ectodomain is the first 100 amino acid residues. In specific embodiments, the SARS-CoV-2 Omicron variant spike protein ectodomain is fused to the NDV F protein transmembrane and cytoplasmic domains via a linker (e.g., GGGGS (SEQ ID NO:24)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO:73), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In other embodiments, the SARS-CoV-2 Omicron variant spike protein is fused directly to the NDV F protein transmembrane and cytoplasmic domains. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein comprising (or consisting of) a derivative of a SARS-CoV-2 Omicron variant spike protein ectodomain with 1, 2, 3, 4, 5, 6, 7, 8, 9, 10, 11, or 12 amino acids deleted, and NDV F protein transmembrane and cytoplasmic domains. In another embodiment, provided herein is a transgene comprising a nucleotide sequence encoding a chimeric F protein comprising (or consisting of) a derivative of a SARS-CoV-2 Omicron variant spike protein ectodomain with 1, 2, 3, 4, 5, 6, 7, 8, 9, 10, 11, or 12 amino acids deleted from the C-terminus, and NDV F protein transmembrane and cytoplasmic domains. In another embodiment, provided herein is a transgene comprising a nucleotide sequence encoding a chimeric F protein comprising (or consisting of) a derivative of a SARS-CoV-2 Omicron variant spike protein ectodomain with 1, 2, 3, 4, 5, 6, 7, 8, 9, 10, 11, or 12 amino acids deleted from the N-terminus, and NDV F protein transmembrane and cytoplasmic domains. In certain embodiments, the derivative of the SARS-CoV-2 Omicron variant spike protein ectodomain lacks the polybasic cleavage site (e.g., one, two or more residues corresponding to amino acid residues 682 to 685 (RRAR) of the spike protein of GenBank Accession No. MN908947.3 are substituted for other amino acid residues). In specific embodiments, the lack of a polybasic cleavage means that the polybasic site is altered such that it cannot be cleaved by, e.g., furin. In a specific embodiment, amino acid residues corresponding to amino acid residues 682 to 685 (RRAR) of the spike protein of GenBank Accession No. MN908947.3 are substituted with a single alanine. In some embodiments, the derivative comprises the following amino acid substitutions at amino acid residues corresponding to at amino acid residues of the spike protein of GenBank Accession No. MN908947.3: F817P, A892P, A899P, A942P, K986P, and V987P. In a specific embodiment, the derivative comprises an amino acid substitution at amino acid residues corresponding to amino acid residues 682 to 685 (RRAR) of the spike protein of GenBank Accession No. MN908947.3 with a single alanine, and the following amino acid substitutions at amino acid residues corresponding to the following amino acid residues of the spike protein of GenBank Accession No. MN908947.3: A67V, T95I, G142D, L212I, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F. In a specific embodiment, the derivative comprises an amino acid substitution at amino acid residues corresponding to amino acid residues 682 to 685 (RRAR) of the spike protein of GenBank Accession No. MN908947.3 with a single alanine, and the following amino acid substitutions at amino acid residues corresponding to the following amino acid residues of the spike protein of GenBank Accession No. MN908947.3: A67V, T95I, G142D, L212I, G339D, S371L, S373P, S375F, K417N, N440K, G446S, S477N, T478K, E484A, Q493R, G496S, Q498R, N501Y, Y505H, T547K, D614G, H655, V687I, N764K, D796Y, N856K, Q954H, N969K, and L981F. In a specific embodiment, the derivative comprises an amino acid substitution at amino acid residues corresponding to amino acid residues 682 to 685 (RRAR) of the spike protein of GenBank Accession No. MN908947.3 with a single alanine, and the following amino acid substitutions at amino acid residues corresponding to the following amino acid residues of the spike protein of GenBank Accession No. MN908947.3: A67V, T95I, G142D, L212I, G339D, S371L, S373P, S375F, K417N, N440K, G446S, S477N, T478K, E484A, Q493, G496S, Q498, N501Y, Y505H, T547K, D614G, H655Y, N679K, P681H, N764K, D796Y, N856K, Q954H, N969K, and L981F. In a specific embodiment, the derivative comprises an amino acid substitution at amino acid residues corresponding to amino acid residues 682 to 685 (RRAR) of the spike protein of GenBank Accession No. MN908947.3 with a single alanine, and amino acid substitutions at amino acid residues corresponding to 1, 2, 3, 4, 5, 6, 7, 8, 9, 10, 11, 12, 13, 14, 15, 16, 17, 18 or more of the amino acid residues of the spike protein of GenBank Accession No. MN908947.3 set forth in Table 5. In a specific embodiment, the derivative comprises: (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P, A942P, K986P, and V987P; and (3) the following amino acid substitutions at amino acid residues corresponding to the following amino acid residues of the spike protein of GenBank Accession No. MN908947.3: A67V, T95I, G142D, L212I, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F. In a specific embodiment, the derivative comprises: (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P, A942P, K986P, and V987P; and (3) the following amino acid substitutions at amino acid residues corresponding to the following amino acid residues of the spike protein of GenBank Accession No. MN908947.3: A67V, T95I, G142D, L212I, G339D, S371L, S373P, S375F, K417N, N440K, G446S, S477N, T478K, E484A, Q493R, G496S, Q498R, N501Y, Y505H, T547K, D614G, H655, V687I, N764K, D796Y, N856K, Q954H, N969K, and L981F. In a specific embodiment, the derivative comprises: (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P, A942P, K986P, and V987P; and (3) the following amino acid substitutions at amino acid residues corresponding to the following amino acid residues of the spike protein of GenBank Accession No. MN908947.3: A67V, T95I, G142D, L212I, G339D, S371L, S373P, S375F, K417N, N440K, G446S, S477N, T478K, E484A, Q493, G496S, Q498, N501Y, Y505H, T547K, D614G, H655Y, N679K, P681H, N764K, D796Y, N856K, Q954H, N969K, and L981F. In a specific embodiment, the derivative comprises: (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P, A942P, K986P, and V987P; and (3) amino acid substitutions at amino acid residues corresponding to 1, 2, 3, 4, 5, 6, 7, 8, 9, 10, 11, 12, 13, 14, 15, 16, 17, 18 or more of the amino acid residues of the spike protein of GenBank Accession No. MN908947.3 set forth in Table 5. In specific embodiments, the derivative of the SARS-CoV-2 Omicron spike protein ectodomain is fused to the NDV F protein transmembrane and cytoplasmic domains via a linker (e.g., GGGGS (SEQ ID NO:24)). In some embodiments, the derivative of the SARS-CoV-2 spike protein ectodomain includes a serine at amino acid positions corresponding to amino acid positions 371, 373, and/or 375 of SEQ ID NO: 104. In some embodiments, the SARS-CoV-2 spike protein ectodomain includes a serine at amino acid positions corresponding to amino acid positions 371, 373, and/or 375 of SEQ ID NO: 104, and a leucine at the amino acid position corresponding to amino acid position 452 of SEQ ID NO: 104. The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO:73), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO: 24). In other embodiments, the derivative of the SARS-CoV-2 Omicron variant spike protein is fused directly to the NDV F protein transmembrane and cytoplasmic domains. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In a specific embodiment, the derivative comprises: (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P, A942P, K986P, and V987P; and (3) the mutations at amino acid residues corresponding to the amino acid residues of one of the constructs set forth in Table 6, 7, 8, 9, 10, or 11. In a specific embodiment, the derivative comprises: (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P, A942P, K986P, and V987P; and (3) mutations at amino acid residues corresponding to the following amino acid residues of GenBank Accession No. MN908947.3: A67V, HV69-70 deletion, T95I, G142D, VYY143-145 deletion, N211 deletion, L212I, ins214EPE, G339D, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F. In a specific embodiment, the derivative comprises: (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P, A942P, K986P, and V987P; and (3) mutations at amino acid residues corresponding to the following amino acid residues of the spike protein of GenBank Accession No. MN908947.3: T19I, del24-26 (LPP), A27S, G142D, V213G, G339D, T376A, D405N, R408S, K417N, N440K, S477N, T478K, E484A, Q493R, Q498R, N501Y, Y505H, D614G, H655Y, N679K, P681H, N764K, D796Y, Q954H, N969K. In a specific embodiment, the derivative comprises: (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P, A942P, K986P, and V987P; and (3) mutations at amino acid residues corresponding to the following amino acid residues of the spike protein of GenBank Accession No. MN908947.3: T19I, del24-26 (LPP), A27S, del69-70 (HV), G142D, V213G, G339D, T376A, D405N, R408S, K417N, N440K, S477N, L452R, T478K, E484A, F486V, Q498R, N501Y, Y505H, D614G, H655Y, N679K, P681H, N764K, D796Y, Q954H, and N969K. In some embodiments, the SARS-CoV-2 spike protein ectodomain includes a serine at amino acid positions corresponding to amino acid positions 371, 373, and/or 375 of SEQ ID NO: 104. In some embodiments, the SARS-CoV-2 spike protein ectodomain includes a serine at amino acid positions corresponding to amino acid positions 371, 373, and/or 375 of SEQ ID NO:104, and a leucine at the amino acid position corresponding to amino acid position 452. In specific embodiments, the derivative of the SARS-CoV-2 Omicron spike protein ectodomain is fused to the NDV F protein transmembrane and cytoplasmic domains via a linker (e.g., GGGGS (SEQ ID NO:24)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO:73), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In other embodiments, the derivative of the SARS-CoV-2 Omicron variant spike protein is fused directly to the NDV F protein transmembrane and cytoplasmic domains. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein comprising (or consisting of) a SARS-CoV-2 Omicron variant spike protein ectodomain with 1, 2, 3, 4, 5, 6, 7, 8, 9, 10, 11, 12, 13, 14, 15, 16, 17, 18, 19, 20, or more mutations (e.g., amino acid substitutions, amino acid deletions, amino acid additions, or a combination thereof), and NDV F protein transmembrane and cytoplasmic domains. In another embodiment, provided herein is a transgene comprising a nucleotide sequence encoding a chimeric F protein comprising (or consisting of) a SARS-CoV-2 Omicron variant spike protein ectodomain with 1, 2, 3, 4, 5, 6, 7, 8, 9, 10, 11, or 12 amino acid substitutions and 1, 2, 3, 4, 5, 6, 7, 8, 9, 10, 11, or 12 amino acids deleted, and NDV F protein transmembrane and cytoplasmic domains. In specific embodiments, the SARS-CoV-2 Omicron variant spike protein ectodomain is fused to the NDV F protein transmembrane and cytoplasmic domains via a linker (e.g., GGGGS (SEQ ID NO:24)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO: 73), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In other embodiments, the SARS-CoV-2 Omicron variant spike protein is fused directly to the NDV F protein transmembrane and cytoplasmic domains. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein comprising (or consisting of) a derivative of a SARS-CoV-2 Omicron variant spike protein ectodomain with 1, 2, 3, 4, 5, 6, 7, 8, 9, 10, 11, 12, 13, 14, 15, 16, 17, 18, 19, 20, or more mutations (e.g., amino acid substitutions, amino acid deletions, amino acid additions, or a combination thereof), and NDV F protein transmembrane and cytoplasmic domains. In another embodiment, provided herein is a transgene comprising a nucleotide sequence encoding a chimeric F protein comprising (or consisting of) a derivative of a SARS-CoV-2 Omicron variant spike protein ectodomain with 1, 2, 3, 4, 5, 6, 7, 8, 9, 10, 11, or 12 amino acid substitutions and 1, 2, 3, 4, 5, 6, 7, 8, 9, 10, 11, or 12 amino acids deleted, and NDV F protein transmembrane and cytoplasmic domains. In certain embodiments, the derivative of the SARS-CoV-2 Omicron variant spike protein ectodomain lacks the polybasic cleavage site (e.g., one, two or more residues corresponding to amino acid residues 682 to 685 (RRAR) of the spike protein of GenBank Accession No. MN908947.3 are substituted for other amino acid residues). In specific embodiments, the lack of a polybasic cleavage means that the polybasic site is altered such that it cannot be cleaved by, e.g., furin. In a specific embodiment, amino acid residues corresponding to amino acid residues 682 to 685 (RRAR) of the spike protein of GenBank Accession No. MN908947.3 are substituted with a single alanine. In a specific embodiment, the derivative comprises: (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of GenBank Accession No. MN908947.3: F817P, A892P, A899P, A942P, K986P, and V987P; and (3) the following mutations at amino acid residues corresponding to the following amino acid residues of GenBank Accession No. MN908947.3: A67V, HV69-70 deletion, T95I, G142D, VYY143-145 deletion, N211 deletion, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F. In a specific embodiment, the derivative comprises: (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of GenBank Accession No. MN908947.3: F817P, A892P, A899P, A942P, K986P, and V987P; and (3) the following mutations at amino acid residues corresponding to the following amino acid residues of GenBank Accession No. MN908947.3: A67V, HV69-70 deletion, T95I, G142D, VYY143-145 deletion, N211 deletion, L212I, ins214EPE, G339D, S371L, S373P, S375F, K417N, N440K, G446S, S477N, T478K, E484A, Q493R, G496S, Q498R, N501Y, Y505H, T547K, D614G, H655, NSPRRARS 679-686 deletion, V687I, N764K, D796Y, N856K, Q954H, N969K, L981F. In a specific embodiment, the derivative comprises: (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of GenBank Accession No. MN908947.3: F817P, A892P, A899P, A942P, K986P, and V987P; and (3) the following mutations at amino acid residues corresponding to the following amino acid residues of GenBank Accession No. MN908947.3: A67V, HV69-70 deletion, T95I, G142D, VYY143-145 deletion, N211 deletion, L212I, ins214EPE, G339D, S371L, S373P, S375F, K417N, N440K, G446S, S477N, T478K, E484A, Q493, G496S, Q498, N501Y, Y505H, T547K, D614G, H655Y, N679K, P681H, N764K, D796Y, N856K, Q954H, N969K, L981F. In a specific embodiment, the derivative comprises: (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of GenBank Accession No. MN908947.3: F817P, A892P, A899P, A942P, K986P, and V987P; and (3) amino acid substitutions at amino acid residues corresponding to 1, 2, 3, 4, 5, 6, 7, 8, or more of the amino acid residues of the spike protein of GenBank Accession No. MN908947.3 set forth in Table 5, 6, 7, 8, 9, 10, or 11. In a specific embodiment, the derivative comprises: (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of GenBank Accession No. MN908947.3: F817P, A892P, A899P, A942P, K986P, and V987P; and (3) the mutations at amino acid residues corresponding to the amino acid residues of one of the constructs set forth in Table 6, 7, 8, 9, 10, or 11. In a specific embodiment, the derivative comprises: (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of GenBank Accession No. MN908947.3: F817P, A892P, A899P, A942P, K986P, and V987P; and (3) mutations at amino acid residues corresponding to the following amino acid residues of the spike protein of GenBank Accession No. MN908947.3: A67V, HV69-70 deletion, T95I, G142D, VYY143-145 deletion, N211 deletion, L212I, ins214EPE, G339D, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F. In a specific embodiment, the derivative comprises: (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P, A942P, K986P, and V987P; and (3) mutations at amino acid residues corresponding to the following amino acid residues of GenBank Accession No. MN908947.3: T19I, del24-26 (LPP), A27S, G142D, V213G, G339D, T376A, D405N, R408S, K417N, N440K, S477N, T478K, E484A, Q493R, Q498R, N501Y, Y505H, D614G, H655Y, N679K, P681H, N764K, D796Y, Q954H, N969K. In a specific embodiment, the derivative comprises: (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P, A942P, K986P, and V987P; and (3) mutations at amino acid residues corresponding to the following amino acid residues of GenBank Accession No. MN908947.3: T19I, del24-26 (LPP), A27S, del69-70 (HV), G142D, V213G, G339D, T376A, D405N, R408S, K417N, N440K, S477N, L452R, T478K, E484A, F486V, Q498R, N501Y, Y505H, D614G, H655Y, N679K, P681H, N764K, D796Y, Q954H, and N969K. In some embodiments, the derivative of the SARS-CoV-2 Omicron spike protein ectodomain includes a serine at amino acid positions corresponding to amino acid positions 371, 373, and/or 375 of SEQ ID NO: 104. In some embodiments, the derivative of the SARS-CoV-2 Omicron spike protein ectodomain includes a serine at amino acid positions corresponding to amino acid positions 371, 373, and/or 375 of SEQ ID NO:104, and a leucine at the amino position corresponding to amino acid position 452 of SEQ ID NO:104. In specific embodiments, the derivative of the SARS-CoV-2 Omicron variant spike protein ectodomain is fused to the NDV F protein transmembrane and cytoplasmic domains via a linker (e.g., GGGGS (SEQ ID NO:24)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO:73), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In other embodiments, the derivative of the SARS-CoV-2 Omicron variant spike protein is fused directly to the NDV F protein transmembrane and cytoplasmic domains. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, described herein are transgenes comprising a nucleotide sequence encoding a chimeric F protein, wherein the chimeric F protein comprises a derivative of a SARS-CoV-2 Omicron variant spike protein ectodomain and NDV F protein transmembrane and cytoplasmic domains, wherein the derivative comprises amino acid residues corresponding to amino acid residues 817, 892, 899, 942, 986, and 987 of the spike protein found at the spike protein of GenBank Accession No. MN908947.3 substituted with prolines, and wherein the derivative lacks a polybasic cleavage site. In specific embodiments, the lack of a polybasic cleavage means that the polybasic site is altered such that it cannot be cleaved by, e.g., furin. The SARS-CoV-2 Omicron variant spike protein ectodomain may lack the polybasic cleavage site as a result of amino acid residues 682 to 685 of the polybasic cleavage site being substituted with a single alanine. See, e.g., Table 2, infra, with the transmembrane and cytoplasmic domains of NDV F protein indicated. In specific embodiments, the derivative of the SARS-CoV-2 Omicron variant spike protein ectodomain is fused to the NDV F protein transmembrane and cytoplasmic domains via a linker (e.g., GGGGS (SEQ ID NO:24)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO:73), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused directly to the derivative of the SARS-CoV-2 spike protein ectodomain. In certain embodiments, the transgene encoding the chimeric F protein is codon optimized. See, e.g., Section 5.1.4, infra, for a discussion regarding codon optimization. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between NDV NP and P transcription units, or between the NDV HN and L transcription units).

In another embodiment, provided herein is a transgene comprising a nucleotide sequence that is at least 85%, at least 90%, at least 95%, at least 96%, at least 97%, at least 98% or at least 99% identical to the nucleotide sequence of SEQ ID NO:6, 7, 10, 11, 14, 15, 30, 36, 42, 48, 54, 60, or 66. In another embodiment, provided herein is a transgene comprising a nucleotide sequence that is at least 95%, at least 96%, at least 97%, at least 98% or at least 99% identical to the nucleotide sequence of SEQ ID NO: 6, 7, 10, 11, 14, 15, 30, 36, 42, 48, 54, 60, or 66. In another embodiment, provided herein is a transgene comprising a nucleotide sequence that is at least 97%, at least 98% or at least 99% identical to the nucleotide sequence of SEQ ID NO: 6, 7, 10, 11, 14, 15, 30, 36, 42, 48, 54, 60, or 66. Methods/techniques known in the art may be used to determine sequence identity (see, e.g., “Best Fit” or “Gap” program of the Sequence Analysis Software Package, version 10; Genetics Computer Group, Inc.). In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In specific embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein, wherein the chimeric F protein comprises (or consists of) a SARS-CoV-2 Omicron variant spike protein ectodomain and NDV F protein transmembrane and cytoplasmic domains. In another embodiment, provided herein is a transgene comprising a nucleotide sequence encoding a chimeric F protein comprising a derivative of a SARS-CoV-2 Omicron variant spike protein ectodomain and NDV F protein transmembrane and cytoplasmic domains, wherein the derivative comprises a SARS-CoV-2 Omicron spike protein ectodomain with 1, 2, 3, 4, 5, 6, 7, 8, 9, 10, 11, 12, 13, 14, or 15 amino acids substituted with another amino acid (e.g., a conservative amino acid substitution) and lacks a polybasic cleavage site (e.g., as a result of one, two, or more amino acid substitutions in polybasic cleavage site), and wherein amino acid residues corresponding to amino acid residues 817, 892, 899, 942, 986, and 987 of the spike protein found at the spike protein of GenBank Accession No. MN908947.3 are substituted with prolines. The SARS-CoV-2 Omicron variant spike protein ectodomain may lack the polybasic cleavage site as a result of a substitution of amino acid residues RRAR to A at amino acid residues corresponding to amino acid residues 682 to 685 of the spike protein of GenBank Accession No. MN908947.3. In certain embodiments, the derivative of the SARS-CoV-2 Omicron variant spike protein ectodomain comprises the amino acid sequence of SEQ ID NO: 19, 21, 23, 33, 35, 39, 41, 45, 47, 51, 53, 57, 59, 63, 65, 69, or 71. In specific embodiments, the derivative of the SARS-CoV-2 Omicron spike protein ectodomain is fused to the NDV F protein transmembrane and cytoplasmic domains via a linker (e.g., GGGGS (SEQ ID NO:24)).

In certain embodiments, the derivative of the SARS-CoV-2 Omicron spike protein ectodomain comprises the amino acid sequence of SEQ ID NO: 19, 21, 23, 33, 35, 39, 41, 45, 47, 51, 53, 57, 59, 63, 65, 69, or 71. In certain embodiments, the derivative of the SARS-CoV-2 Omicron variant spike protein ectodomain comprises an amino acid sequence that is at least 90%, at least 95%, at least 96%, at least 97%, at least 98%, or at least 99% identical to the amino acid sequence of SEQ ID NO: 19, 21, 23, 33, 35, 39, 41, 45, 47, 51, 53, 57, 59, 63, 65, 69, or 71. In some embodiments, the derivative of the SARS-CoV-2 Omicron spike protein ectodomain comprises two or more (e.g., 1, 2, 3, 4, 5, 6, or 7), or all of the amino acid modifications at amino acid positions corresponding to the indicated amino acid positions of SEQ ID NO: 104: N440K, S477N, Y505H, N679K, N764K, D796Y, Q954H, and/or N969K. In certain embodiments, the derivative of the SARS-CoV-2 Omicron spike protein ectodomain is encoded by nucleotide sequence that is at least 80%, at least 85%, or at least 90% identical to the nucleotide sequence of SEQ ID NO: 18, 20, 22, 32, 34, 38, 40, 44, 46, 50, 52, 56, 58, 62, 64, 68, or 70. In certain embodiments, the derivative of the SARS-CoV-2 Omicron spike protein ectodomain is encoded by a nucleotide sequence that is at least 95%, at least 98%, or at least 99% identical to the nucleotide sequence of SEQ ID NO: 18, 20, 22, 32, 34, 38, 40, 44, 46, 50, 52, 56, 58, 62, 64, 68, or 70. In certain embodiments, the derivative of the SARS-CoV-2 Omicron spike protein ectodomain is encoded by the nucleotide sequence of SEQ ID NO: 18, 20, 22, 32, 34, 38, 40, 44, 46, 50, 52, 56, 58, 62, 64, 68, or 70. In some embodiments, a derivative of the SARS-CoV-2 Omicron spike protein ectodomain comprises the amino acid sequence of GenBank Accession No. MN908947.3, with (1) an amino acid substitution at amino acid residues 682 to 685 (RRAR) of the spike protein of GenBank Accession No. MN908947.3 with a single alanine; (2) amino acid substitutions at amino acid residues F817P, A892P, A899P, A942P, K986P, and V987P of the spike protein of GenBank Accession No. MN908947.3; and (3) the following amino acid substitutions at the following amino acid residues of the spike protein of GenBank Accession No. MN908947.3: A67V, T95I, G142D, L212I, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F. In some embodiments, a derivative of the SARS-CoV-2 Omicron spike protein ectodomain comprises the amino acid sequence of GenBank Accession No. MN908947.3, with (1) an amino acid substitution at amino acid residues 682 to 685 (RRAR) of the spike protein of GenBank Accession No. MN908947.3 with a single alanine; (2) amino acid substitutions at amino acid residues F817P, A892P, A899P, A942P, K986P, and V987P of the spike protein of GenBank Accession No. MN908947.3; and (3) the following amino acid substitutions at the following amino acid residues of the spike protein of GenBank Accession No. MN908947.3: A67V, T95I, G142D, L212I, G339D, S371L, S373P, S375F, K417N, N440K, G446S, S477N, T478K, E484A, Q493R, G496S, Q498R, N501Y, Y505H, T547K, D614G, H655, V687I, N764K, D796Y, N856K, Q954H, N969K, and L981F. In some embodiments, a derivative of the SARS-CoV-2 Omicron spike protein ectodomain comprises the amino acid sequence of the spike protein of GenBank Accession No. MN908947.3, with (1) an amino acid substitution at amino acid residues 682 to 685 (RRAR) of the spike protein of GenBank Accession No. MN908947.3 with a single alanine; (2) amino acid substitutions at amino acid residues F817P, A892P, A899P, A942P, K986P, and V987P of the spike protein of GenBank Accession No. MN908947.3; and (3) the following amino acid substitutions at the following amino acid residues of the spike protein of GenBank Accession No. MN908947.3: A67V, T95I, G142D, L212I, G339D, S371L, S373P, S375F, K417N, N440K, G446S, S477N, T478K, E484A, Q493, G496S, Q498, N501Y, Y505H, T547K, D614G, H655Y, N679K, P681H, N764K, D796Y, N856K, Q954H, N969K, and L981F. In some embodiments, a derivative of the SARS-CoV-2 Omicron spike protein ectodomain comprises the amino acid sequence of the spike protein of GenBank Accession No. MN908947.3, with (1) an amino acid substitution at amino acid residues 682 to 685 (RRAR) of the spike protein of GenBank Accession No. MN908947.3 with a single alanine; (2) amino acid substitutions at amino acid residues F817P, A892P, A899P, A942P, K986P, and V987P of GenBank Accession No. MN908947.3; and (3) 1, 2, 3, 4, 5, 6, 7, 8, 9, 10, 11, 12 or more of the amino acid substitutions at the amino acid residues of the spike protein of GenBank Accession No. MN908947.3 set forth in Table 5, 6, 7, 8, 9, 10, or 11. In some embodiments, a derivative of the SARS-CoV-2 Omicron spike protein ectodomain comprises the amino acid sequence of the spike protein of GenBank Accession No. MN908947.3, with (1) an amino acid substitution at amino acid residues 682 to 685 (RRAR) of the spike protein of GenBank Accession No. MN908947.3 with a single alanine; (2) amino acid substitutions at amino acid residues F817P, A892P, A899P, A942P, K986P, and V987P of the spike protein of GenBank Accession No. MN908947.3; and (3) the following amino acid mutations at the following amino acid residues of the spike protein of GenBank Accession No. MN908947.3: A67V, HV69-70 deletion, T95I, G142D, VYY143-145 deletion, N211 deletion, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F. In some embodiments, a derivative of the SARS-CoV-2 Omicron spike protein ectodomain comprises the amino acid sequence of GenBank Accession No. MN908947.3, with (1) an amino acid substitution at amino acid residues 682 to 685 (RRAR) of the spike protein of GenBank Accession No. MN908947.3 with a single alanine; (2) amino acid substitutions at amino acid residues F817P, A892P, A899P, A942P, K986P, and V987P of the spike protein of GenBank Accession No. MN908947.3; and (3) the following amino acid mutations at the following amino acid residues of the spike protein of GenBank Accession No. MN908947.3: A67V, HV69-70 deletion, T95I, G142D, VYY143-145 deletion, N211 deletion, L212I, ins214EPE, G339D, S371L, S373P, S375F, K417N, N440K, G446S, S477N, T478K, E484A, Q493R, G496S, Q498R, N501Y, Y505H, T547K, D614G, H655, NSPRRARS 679-686 deletion, V687I, N764K, D796Y, N856K, Q954H, N969K, L981F. In some embodiments, a derivative of the SARS-CoV-2 Omicron spike protein ectodomain comprises the amino acid sequence of the spike protein of GenBank Accession No. MN908947.3, with (1) an amino acid substitution at amino acid residues 682 to 685 (RRAR) of the spike protein of GenBank Accession No. MN908947.3 with a single alanine; (2) amino acid substitutions at amino acid residues F817P, A892P, A899P, A942P, K986P, and V987P of the spike protein of GenBank Accession No. MN908947.3; and (3) the following amino acid mutations at the following amino acid residues of the spike protein of GenBank Accession No. MN908947.3: A67V, HV69-70 deletion, T95I, G142D, VYY143-145 deletion, N211 deletion, L212I, ins214EPE, G339D, S371L, S373P, S375F, K417N, N440K, G446S, S477N, T478K, E484A, Q493, G496S, Q498, N501Y, Y505H, T547K, D614G, H655Y, N679K, P681H, N764K, D796Y, N856K, Q954H, N969K, L981F. In some embodiments, a derivative of the SARS-CoV-2 Omicron spike protein ectodomain comprises the amino acid sequence of the spike protein of GenBank Accession No. MN908947.3, with (1) an amino acid substitution at amino acid residues 682 to 685 (RRAR) of the spike protein of GenBank Accession No. MN908947.3 with a single alanine; (2) amino acid substitutions at amino acid residues F817P, A892P, A899P, A942P, K986P, and V987P of the spike protein of GenBank Accession No. MN908947.3; and (3) 1, 2, 3, 4, 5, 6, 7, 8, 9, 10, 11, 12 or more of the amino acid mutations at the amino acid residues of the spike protein of GenBank Accession No. MN908947.3 set forth in Table 5, 6, 7, 8, 9, 10, or 11. In a specific embodiment, the derivative comprises: (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P, A942P, K986P, and V987P; and (3) the mutations at amino acid residues corresponding to the amino acid residues of one of the constructs set forth in Table 6, 7, 8, 9, 10, or 11. In a specific embodiment, the derivative comprises: (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P, A942P, K986P, and V987P; and (3) mutations at amino acid residues corresponding to the following amino acid residues of the spike protein of GenBank Accession No. MN908947.3: A67V, HV69-70 deletion, T95I, G142D, VYY143-145 deletion, N211 deletion, L212I, ins214EPE, G339D, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F. In a specific embodiment, the derivative comprises: (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P, A942P, K986P, and V987P; and (3) mutations at amino acid residues corresponding to the following amino acid residues of the spike protein of GenBank Accession No. MN908947.3: T19I, del24-26 (LPP), A27S, G142D, V213G, G339D, T376A, D405N, R408S, K417N, N440K, S477N, T478K, E484A, Q493R, Q498R, N501Y, Y505H, D614G, H655Y, N679K, P681H, N764K, D796Y, Q954H, N969K. In a specific embodiment, the derivative comprises: (1) an amino acid substitution at amino acid residues corresponding to amino acid residues 682 to 685 (RRAR) of the spike protein of GenBank Accession No. MN908947.3 with a single alanine; (2) amino acid substitutions at amino acid residues corresponding to the following amino acid residues of the spike protein of GenBank Accession No. MN908947.3: F817P, A892P, A899P, A942P, K986P, and V987P; and (3) mutations at amino acid residues corresponding to the following amino acid residues of the spike protein of GenBank Accession No. MN908947.3: T19I, del24-26 (LPP), A27S, del69-70 (HV), G142D, V213G, G339D, T376A, D405N, R408S, K417N, N440K, S477N, L452R, T478K, E484A, F486V, Q498R, N501Y, Y505H, D614G, H655Y, N679K, P681H, N764K, D796Y, Q954H, and N969K. In some embodiments, the derivative of the SARS-CoV-2 Omicron variant spike protein ectodomain includes a serine at amino acid positions corresponding to amino acid positions 371, 373, and/or 375 of SEQ ID NO: 104. In some embodiments, the derivative of the SARS-CoV-2 Omicron variant spike protein ectodomain includes a serine at amino acid positions corresponding to amino acid positions 371, 373, and/or 375 of SEQ ID NO: 104, and a leucine at the amino acid position corresponding to 452 of SEQ ID NO: 104.

In specific embodiments, the derivative of the SARS-CoV-2 Omicron variant spike protein ectodomain is fused to the NDV F protein transmembrane and cytoplasmic domains via a linker (e.g., GGGGS (SEQ ID NO:24)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO:73), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In other embodiments, the derivative of the SARS-CoV-2 Omicron variant spike protein ectodomain is fused directly to the NDV F protein transmembrane and cytoplasmic domains.

In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein comprising (or consisting of) a derivative of a SARS-CoV-2 Omicron variant spike protein ectodomain and NDV F protein transmembrane and cytoplasmic domains, wherein the derivative comprises a SARS-CoV-2 spike protein ectodomain with 1, 2, 3, 4, 5, 6, 7, 8, 9, 10, 11, or 12 amino acids deleted and lacks a polybasic cleavage site (e.g., as a result of one, two, or more amino acid substitutions in polybasic cleavage site), and wherein amino acid residues corresponding to amino acid residues 817, 892, 899, 942, 986, and 987 of the spike protein found at GenBank Accession No. MN908947.3 are substituted with prolines. In specific embodiments, the lack of a polybasic cleavage means that the polybasic site is altered such that it cannot be cleaved by, e.g., furin. The derivative of the SARS-CoV-2 Omicron variant spike protein ectodomain may lack the polybasic cleavage site as a result of a substitution of amino acid residues RRAR to A at amino acid residues corresponding to amino acid residues 682 to 685 of the spike protein of GenBank Accession No. MN908947.3. In specific embodiments, the derivative of the SARS-CoV-2 Omicron variant spike protein ectodomain is fused to the NDV F protein transmembrane and cytoplasmic domains via a linker (e.g., GGGGS (SEQ ID NO: 24)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO:73), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In other embodiments, the derivative of the SARS-CoV-2 Omicron variant spike protein is fused directly to the NDV F protein transmembrane and cytoplasmic domains. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In specific embodiments, the NDV F protein transmembrane and cytoplasmic domains of the chimeric F protein are from a different NDV strain than the transcription units of the NDV genome. In a specific embodiment, the NDV genome is of the LaSota strain.

In another specific embodiment, provided herein is a transgene comprising a nucleotide sequence encoding a chimeric F protein, wherein chimeric F protein comprises a derivative of SARS-CoV-2 Omicron variant spike protein ectodomain and NDV F protein transmembrane and cytoplasmic domains, wherein the derivative of SARS-CoV-2 Omicron variant spike protein ectodomain is encoded by a nucleotide sequence that can hybridize under high, moderate or typical stringency hybridization conditions to the nucleic acid sequence set forth in SEQ ID NO: 18, 20, 22, 32, 34, 38, 40, 44, 46, 50, 52, 56, 58, 62, 64, 68, 70, 76, 78, 84, 94, 96, 100, or 102. Hybridization conditions are known to one of skill in the art (see, e.g., U.S. Patent Application No. 2005/0048549 at, e.g., paragraphs 72 and 73). In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein comprising (or consisting of) a derivative of a SARS-CoV-2 Omicron variant spike protein ectodomain and NDV F protein transmembrane and cytoplasmic domains, wherein the derivative comprises a SARS-CoV-2 Omicron variant spike protein with 1, 2, 3, 4, 5, 6, 7, 8, 9, 10, 11, 12, 13, 14 or 15 mutations (e.g. amino acid substitutions, amino acid additions, amino acid deletions or a combination thereof) and lacks a polybasic cleavage site (e.g., as a result of one, two, or more amino acid substitutions in polybasic cleavage site), and wherein amino acid residues corresponding to amino acid residues 817, 892, 899, 942, 986, and 987 of the spike protein found at GenBank Accession No. MN908947.3 are substituted with prolines. In specific embodiments, the lack of a polybasic cleavage means that the polybasic site is altered such that it cannot be cleaved by, e.g., furin. The derivative of the SARS-CoV-2 Omicron variant spike protein ectodomain may lack the polybasic cleavage site as a result of a substitution of amino acid residues RRAR to A at amino acid residues corresponding to amino acid residues 682 to 685 of the spike protein of GenBank Accession No. MN908947.3. In certain embodiments, the derivative of the SARS-CoV-2 Omicron variant spike protein ectodomain comprises an amino acid sequence that is at least 90%, at least 95%, at least 96%, at least 97%, at least 98%, or at least 99% identical to the amino acid sequence of SEQ ID NO: 19, 21, 23, 33, 35, 39, 41, 45, 47, 51, 53, 57, 59, 63, 65, 69, 71, 77, 79, 83, 85, 89, 91, 95, 97, 101, or 103. In certain embodiments, the derivative of the SARS-CoV-2 Omicron variant spike protein ectodomain comprises the amino acid sequence of SEQ ID NO: 19, 21, 23, 33, 35, 39, 41, 45, 47, 51, 53, 57, 59, 63, 65, 69, 71, 77, 79, 83, 85, 89, 91, 95, 97, 101, or 103 . . . . In specific embodiments, the derivative of the SARS-CoV-2 Omicron variant spike protein ectodomain is fused to the NDV F protein transmembrane and cytoplasmic domains via a linker (e.g., GGGGS (SEQ ID NO:24)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO:73), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In other embodiments, the derivative of the SARS-CoV-2 Omicron variant spike protein is fused directly to the NDV F protein transmembrane and cytoplasmic domains. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another specific embodiment, provided herein is a transgene comprising a nucleotide sequence that can hybridize under high, moderate or typical stringency hybridization conditions to the nucleic acid sequence set forth in SEQ ID NO:6, 7, 10, 11, 14 15, 30, 36, 42, 48, 54, 60, 66, 74, 80, 86, 92, or 98. Hybridization conditions are known to one of skill in the art (see, e.g., U.S. Patent Application No. 2005/0048549 at, e.g., paragraphs 72 and 73). In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene that comprises a nucleotide sequence encoding a chimeric F protein, wherein the chimeric F protein comprises a derivative of a SARS-CoV-2 Omicron variant spike protein ectodomain and an NDV F protein transmembrane and cytoplasmic domains, wherein the derivative comprises the amino acid sequence set forth in SEQ ID NO: 19, 21, 23, 33, 35, 39, 41, 45, 47, 51, 53, 57, 59, 63, 65, 69, 71, 77, 79, 83, 85, 89, 91, 95, 97, 101, or 103 . . . . In another embodiment, provided herein is a transgene that comprises a nucleotide sequence encoding a chimeric F protein, wherein the chimeric F protein comprises a derivative of a SARS-CoV-2 Omicron variant spike protein ectodomain and an NDV F protein transmembrane and cytoplasmic domains, wherein the derivative comprises an amino acid sequence that is at least 85%, at least 90%, or at least 95%, identical to the amino acid sequence set forth in SEQ ID NO: 19, 21, 23, 33, 35, 39, 41, 45, 47, 51, 53, 57, 59, 63, 65, 69, 71, 77, 79, 83, 85, 89, 91, 95, 97, 101, or 103 . . . . In another embodiment, provided herein is a transgene that comprises a nucleotide sequence encoding a chimeric F protein, wherein the chimeric F protein comprises a derivative of a SARS-CoV-2 Omicron variant spike protein ectodomain and an NDV F protein transmembrane and cytoplasmic domains, wherein the derivative comprises an amino acid sequence that is at least 96%, at least 97%, or at least 98%, at least 99%, or at least 99.5% identical to the amino acid sequence set forth in SEQ ID NO: 19, 21, 23, 33, 35, 39, 41, 45, 47, 51, 53, 57, 59, 63, 65, 69, 71, 77, 79, 83, 85, 89, 91, 95, 97, 101, or 103 . . . . See, e.g., Table 2, infra, with the transmembrane and cytoplasmic domains of NDV F protein indicated. In specific embodiments, the derivative of the SARS-CoV-2 Omicron variant spike protein ectodomain is fused to the NDV F protein transmembrane and cytoplasmic domains via a linker (e.g., GGGGS (SEQ ID NO:24)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO:73), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused directly to the derivative of the SARS-CoV-2 Omicron variant spike protein ectodomain. In certain embodiments, the transgene encoding the chimeric F protein is codon optimized. See, e.g., Section 5.1.4, infra, for a discussion regarding codon optimization. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units).

In another embodiment, provided herein is a transgene that comprises a nucleotide sequence encoding a chimeric F protein, wherein the chimeric F protein comprises a derivative of a SARS-CoV-2 Omicron variant spike protein ectodomain and an NDV F protein transmembrane and cytoplasmic domains, wherein the derivative is encoded by a nucleotide sequence that is at least 80%, at least 85%, or at least 90% identical to the nucleotide sequence of SEQ ID NO: 18, 20, 22, 32, 34, 38, 40, 44, 46, 50, 52, 56, 58, 62, 64, 68, 70, 76, 78, 82, 85, 88, 90, 94, 97, 100, or 102. In another embodiment, provided herein is a transgene that comprises a nucleotide sequence encoding a chimeric F protein, wherein the chimeric F protein comprises a derivative of a SARS-CoV-2 Omicron variant spike protein ectodomain and an NDV F protein transmembrane and cytoplasmic domains, wherein the derivative is encoded by a nucleotide sequence that is at least 95%, at least 98%, or at least 99% identical to the nucleotide sequence of SEQ ID NO: 18, 20, 22, 32, 34, 38, 40, 44, 46, 50, 52, 56, 58, 62, 64, 68, or 70. See, e.g., Table 2, infra, with the transmembrane and cytoplasmic domains of NDV F protein indicated. In specific embodiments, the derivative of the SARS-CoV-2 Omicron variant spike protein ectodomain is fused to the NDV F protein transmembrane and cytoplasmic domains via a linker (e.g., GGGGS (SEQ ID NO:24)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO: 73), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:24). In some embodiments, the NDV F protein transmembrane and cytoplasmic domains are fused directly to the derivative of the SARS-CoV-2 Omicron variant spike protein ectodomain. In certain embodiments, the transgene encoding the chimeric F protein is codon optimized. See, e.g., Section 5.1.4, infra, for a discussion regarding codon optimization. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units).

In another embodiment, provided herein is a transgene that comprises a nucleotide sequence encoding a chimeric F protein, wherein the chimeric F protein comprises the amino acid sequence of SEQ ID NO:8, 9, 12, 13, 16, 17, 31, 37, 43, 49, 55, 61, 67, 75, 81, 87, 93, or 99. In another embodiment, provided herein is a transgene that comprises a nucleotide sequence encoding a chimeric F protein, wherein the chimeric F protein comprises an amino acid sequence that is at least 85%, at least 90%, or at least 95%, identical to the amino acid sequence set forth in SEQ ID NO: 8, 9, 12, 13, 16, 17, 31, 37, 43, 49, 55, 61, 67, 75, 81, 87, 93, or 99. In another embodiment, provided herein is a transgene that comprises a nucleotide sequence encoding a chimeric F protein, wherein the chimeric F protein comprises an amino acid sequence that is at least 96%, at least 97%, or at least 98%, at least 99%, or at least 99.5% identical to the amino acid sequence set forth in SEQ ID NO: 8, 9, 12, 13, 16, 17, 31, 37, 43, 49, 55, 61, 67, 75, 81, 87, 93, or 99. In certain embodiments, the transgene encoding the chimeric F protein is codon optimized. See, e.g., Section 5.1.4, infra, for a discussion regarding codon optimization. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units).

In another embodiment, provided herein is a transgene that comprises a nucleotide sequence of SEQ ID NO: 6, 7, 10, 11, 14, 15, 30, 36, 42, 48, 54, 60, 66, 74, 80, 86, 92, or 98. In another embodiment, provided herein is a transgene that comprises a nucleotide sequence that is at least 85%, at least 90%, or at least 95%, identical to the nucleotide sequence of SEQ ID NO: 6, 7, 10, 11, 14, 15, 30, 36, 42, 48, 54, 60, or 66. In another embodiment, provided herein is a transgene that comprises a nucleotide sequence comprises an nucleotide sequence that is at least 96%, at least 97%, or at least 98%, at least 99%, or at least 99.5% identical to the nucleotide sequence set forth in SEQ ID NO: 6, 7, 10, 11, 14, 15, 30, 36, 42, 48, 54, 60, 66, 74, 80, 86, 92, or 98. In certain embodiments, the transgene encoding the chimeric F protein is codon optimized. See, e.g., Section 5.1.4, infra, for a discussion regarding codon optimization. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units).

In a specific embodiment, a transgene encodes a protein described herein. In a specific embodiment, a transgene encoding a chimeric F protein is one described in the Example (Section 6), infra. In a specific embodiment, a transgene comprises a nucleotide sequence encoding the ectodomain of a chimeric F protein described in the Example (Section 6), infra. In a specific embodiment, a transgene comprises a nucleotide sequence described in Table 3, infra. In a specific embodiment, a transgene encodes a protein comprising an amino acid sequence described in Table 3, infra. In a specific embodiment, a transgene encodes a chimeric F protein comprising an amino acid sequence described in Table 3, infra. In some embodiments, a protein (e.g., a chimeric F protein) is one encoded by a transgene described herein. In some embodiments, provided herein is a recombinant protein encoded by a transgene described herein, a polynucleotide described herein, nucleic acid sequence described herein, or nucleotide sequence described herein. In a specific embodiment, a chimeric F protein is one described in Section 6, infra. In some embodiments, provided herein is a recombinant protein comprising (or consisting of) an amino acid described herein (e.g., in Table 3, infra). In a specific embodiment, a chimeric F protein comprises an amino acid sequence described in Table 3, infra.

In specific embodiments, NDV F protein transmembrane and cytoplasmic domains of a chimeric F protein may be from any NDV strain known in the art or described herein. For example, NDV F protein transmembrane and cytoplasmic domains of a chimeric F protein may be from the NDV F protein of LaSota strain, Hitchner B1 strain, Fuller strain, Ulster strain, Roakin strain, or Komarov strain. In some embodiments, the NDV F protein transmembrane and cytoplasmic domains comprise the amino acid sequence of SEQ ID NO: 5.

In certain embodiments, a transgene encoding a protein comprising (or consisting of) a SARS-CoV-2 Omicron variant spike protein or portion thereof (e.g., the ectodomain, S1 domain, S2 domain, or receptor binding domain of SARS-CoV-2 Omicron spike protein) comprises NDV regulatory signals (e.g., gene end, intergenic, and gene start sequences) and Kozak sequences. In some embodiments, a transgene encoding a protein comprising (or consisting of) the ectodomain of a SARS-CoV-2 Omicron variant spike protein comprises NDV regulatory signals (e.g., gene end, intergenic, and gene start sequences) and Kozak sequences. In certain embodiments, a transgene encoding a protein comprising (or consisting of) a derivative of SARS-CoV-2 Omicron variant spike protein or portion thereof (e.g., the ectodomain, S1 domain, S2 domain, or receptor binding domain of SARS-CoV-2 Omicron variant spike protein) comprises NDV regulatory signals (e.g., gene end, intergenic, and gene start sequences) and Kozak sequences. In some embodiments, a transgene encoding a protein comprising (or consisting of) a derivative of the ectodomain of a SARS-CoV-2 Omicron variant spike protein comprises NDV regulatory signals (e.g., gene end, intergenic, and gene start sequences) and Kozak sequences. In certain embodiments, a transgene encoding a chimeric F protein comprises NDV regulatory signals (e.g., gene end, intergenic, and gene start sequences) and Kozak sequences. In some embodiments, a transgene encoding a protein comprising (or consisting of) a SARS-CoV-2 Omicron variant spike protein or portion thereof (e.g., the ectodomain, S1 domain, S2 domain, or receptor binding domain of SARS-CoV-2 Omicron variant spike protein), or a chimeric F protein comprises NDV regulatory signals (e.g., gene end, intergenic, and gene start sequences), Kozak sequences and restriction sites to facilitate cloning. In some embodiments, a transgene encoding a protein comprising (or consisting of) a derivative of a SARS-CoV-2 Omicron variant spike protein or portion thereof (e.g., the ectodomain, S1 domain, S2 domain, or receptor binding domain of SARS-CoV-2 Omicron variant spike protein) comprises NDV regulatory signals (e.g., gene end, intergenic, and gene start sequences), Kozak sequences and restriction sites to facilitate cloning. In some embodiments, a transgene encoding a chimeric F protein comprises NDV regulatory signals (e.g., gene end, intergenic, and gene start sequences), Kozak sequences and restriction sites to facilitate cloning. In certain embodiments, a transgene encoding a protein comprising (or consisting of) a SARS-CoV-2 Omicron variant spike protein or portion thereof (e.g., the ectodomain, S1 domain, S2 domain, or receptor binding domain of SARS-CoV-2 Omicron variant spike protein) comprises NDV regulatory signals (gene end, intergenic and gene start sequences), Kozak sequences, restriction sites to facilitate cloning, and additional nucleotides in the non-coding region to ensure compliance with the rule of six. In some embodiments, a transgene encoding a protein comprising (or consisting of) the ectodomain of a SARS-CoV-2 Omicron variant spike protein comprises NDV regulatory signals (gene end, intergenic and gene start sequences), Kozak sequences, restriction sites to facilitate cloning, and additional nucleotides in the non-coding region to ensure compliance with the rule of six. In certain embodiments, a transgene encoding a protein comprising (or consisting of) a derivative of a SARS-CoV-2 Omicron variant spike protein or portion thereof (e.g., the ectodomain, S1 domain, S2 domain, or receptor binding domain of SARS-CoV-2 Omicron spike protein) comprises NDV regulatory signals (gene end, intergenic and gene start sequences), Kozak sequences, restriction sites to facilitate cloning, and additional nucleotides in the non-coding region to ensure compliance with the rule of six. In some embodiments, a transgene encoding a protein comprising (or consisting of) a derivative of the ectodomain of a SARS-CoV-2 Omicron variant spike protein comprises NDV regulatory signals (gene end, intergenic and gene start sequences), Kozak sequences, restriction sites to facilitate cloning, and additional nucleotides in the non-coding region to ensure compliance with the rule of six. In certain embodiments, a transgene encoding a protein comprising (or consisting of) a chimeric F protein comprises NDV regulatory signals (gene end, intergenic and gene start sequences), Kozak sequences, restriction sites to facilitate cloning, and additional nucleotides in the non-coding region to ensure compliance with the rule of six. See, e.g., SEQ ID NOS: 25-28 for examples of a restriction sequence (SacII), a gene end sequence, a gene start sequence and a Kozak sequence that may be used. In a preferred embodiment, the transgene complies with the rule of six.

In some embodiments, provided herein is a vector (e.g., a plasmid or viral vector) comprising a transgene, or nucleic acid described herein, nucleotide sequence described herein, or polynucleotide sequence described herein.

In a specific embodiment, a transgene described herein is isolated. In specific embodiments, a polynucleotide or nucleic acid sequence described herein is isolated. In certain embodiments, an “isolated” nucleic acid sequence or polynucleotide refers to a nucleic acid molecule which is separated from other nucleic acid molecules which are present in the natural source of the nucleic acid. In other words, the isolated nucleic acid sequence or polynucleotide can comprise heterologous nucleic acids that are not associated with it in nature. In other embodiments, an “isolated” nucleic acid sequence or polynucleotide, such as a cDNA or RNA sequence, can be substantially free of other cellular material, or culture medium when produced by recombinant techniques, or substantially free of chemical precursors or other chemicals when chemically synthesized. The term “substantially free of cellular material” includes preparations of nucleic acid sequences or polynucleotides in which the nucleic acid sequence or polynucleotide is separated from cellular components of the cells from which it is isolated or recombinantly produced. Thus, a nucleic acid sequence or polynucleotide that is substantially free of cellular material includes preparations of nucleic acid sequence or polynucleotide having less than about 30%, 20%, 10%, or 5% (by dry weight) of other nucleic acids. The term “substantially free of culture medium” includes preparations of nucleic acid sequence or polynucleotide in which the culture medium represents less than about 50%, 20%, 10%, or 5% of the volume of the preparation. The term “substantially free of chemical precursors or other chemicals” includes preparations in which the nucleic acid sequence or polynucleotide is separated from chemical precursors or other chemicals which are involved in the synthesis of the nucleic acid sequence or polynucleotide. In specific embodiments, such preparations of the nucleic acid sequence or polynucleotide have less than about 50%, 30%, 20%, 10%, 5% (by dry weight) of chemical precursors or compounds other than the nucleic acid sequence of interest or polynucleotide of interest.

Also, provided herein is a protein (e.g., a recombinant protein) encoded by a polynucleotide described herein, a nucleic acid sequence described herein, a nucleotide sequence described herein, or a transgene described herein. A protein described herein may be isolated from a cell (e.g., a cell line or primary cell) or embryonated egg (e.g., embryonated chicken egg). An “isolated” protein is a protein which is substantially separated from other proteins.

In specific embodiments, a protein described herein comprising a SARS-CoV-2 ectodomain or a derivative thereof has a pre-fusion conformation of a SARS-CoV-2 spike protein. In some embodiments, a chimeric F protein described herein comprising a SARS-CoV-2 ectodomain or a derivative thereof has a post-fusion conformation of a SARS-CoV-2 spike protein.

An “isolated” protein is one which is separated from other proteins which are present in the natural source of the protein. Moreover, an “isolated” protein can be substantially free of other cellular material, or culture medium when produced by recombinant techniques, or substantially free of chemical precursors or other chemicals when chemically synthesized.

5.1.3 Recombinant NDV Encoding a SARS-CoV-2 Spike Protein or a Chimeric F Protein with a SARS-CoV-2 Spike Protein Ectodomain

In one aspect, presented herein are recombinant Newcastle disease virus (“NDV”) comprising a packaged genome, wherein the packaged genome comprises a transgene described herein. In one embodiment, a recombinant NDV comprises a packaged genome, wherein the packaged genome comprises a transgene encoding a SARS-CoV-2 Omicron variant spike protein or portion thereof (e.g., the ectodomain, S1 domain, S2 domain, or receptor binding domain of SARS-CoV-2 Omicron variant spike protein), or derivative thereof. See, e.g., Sections 5.1.2 and 6 for transgenes encoding a SARS-CoV-2 Omicron variant spike protein or portion thereof (e.g., the ectodomain, S1 domain, S2 domain, or receptor binding domain of SARS-CoV-2 Omicron variant spike protein) which the packaged genome may comprise. In a specific embodiment, the transgene encoding a SARS-CoV-2 Omicron variant spike protein or portion thereof (e.g., the ectodomain, S1 domain, S2 domain, or receptor binding domain of SARS-CoV-2 Omicron spike protein), or derivative thereof is one described in Section 5.1.2, supra. In a specific embodiment, the SARS-CoV-2 Omicron variant spike protein or portion thereof (e.g., the ectodomain, S1 domain, S2 domain, or receptor binding domain of SARS-CoV-2 Omicron variant spike protein), or derivative thereof is expressed by cells infected with the recombinant NDV. In certain embodiments, the SARS-CoV-2 Omicron variant spike protein or portion thereof (e.g., the ectodomain, S1 domain, S2 domain, or receptor binding domain of SARS-CoV-2 Omicron spike protein) is incorporated into the NDV virion.

In another embodiment, a recombinant NDV comprises a packaged genome, wherein the packaged genome comprises a transgene encoding a protein comprising a SARS-CoV-2 Omicron variant spike protein or portion thereof (e.g., the ectodomain, S1 domain, S2 domain, or receptor binding domain of SARS-CoV-2 Omicron variant spike protein), or derivative thereof. See, e.g., Sections 5.1.2 and 6 for transgenes encoding a SARS-CoV-2 Omicron variant spike protein or portion thereof (e.g., the ectodomain, S1 domain, S2 domain, or receptor binding domain of SARS-CoV-2 Omicron variant spike protein), or a derivative thereof which the packaged genome may comprise. In a specific embodiment, the transgene is one described in Section 5.1.2 or 6. In a specific embodiment, the SARS-CoV-2 Omicron variant spike protein or portion thereof (e.g., the ectodomain, S1 domain, S2 domain, or receptor binding domain of SARS-CoV-2 Omicron variant spike protein), or derivative thereof is expressed by cells infected with the recombinant NDV. In certain embodiments, the SARS-CoV-2 Omicron variant spike protein or portion thereof (e.g., the ectodomain, S1 domain, S2 domain, or receptor binding domain of SARS-CoV-2 Omicron variant spike protein), or derivative thereof is incorporated into the NDV virion.

In another embodiment, described herein are recombinant NDV comprising a packaged genome, wherein the packaged genome comprises a transgene encoding a protein described herein. In another embodiment, described herein are recombinant NDV comprising a packaged genome, wherein the packaged genome comprises a transgene encoding a chimeric F protein described herein. In a specific embodiment, the chimeric F protein is expressed by cells infected with the recombinant NDV. In another specific embodiment, the chimeric F protein is incorporated into the NDV virion. In another specific embodiment, the chimeric F protein is expressed by cells infected with the recombinant NDV and the chimeric F protein is incorporated into the NDV virion.

In a specific embodiment, a recombinant NDV is one described in the Example (Section 6), infra. In specific embodiments, a recombinant NDV described herein is replication competent. In other embodiments, a recombinant NDV described herein has been inactivated.

In some embodiments, the genome of the recombinant NDV comprises a heterologous sequence encoding a heterologous protein in addition to nucleotide sequence encoding a SARS-CoV-2 Omicron variant spike protein or portion thereof (e.g., the ectodomain, S1 domain, S2 domain, or receptor binding domain of SARS-CoV-2 Omicron variant spike protein), or a derivative thereof. In some embodiments, the genome of the recombinant NDV comprises a heterologous sequence encoding a heterologous protein in addition to nucleotide sequence encoding a chimeric F protein.

In certain embodiments, the genome of the recombinant NDV does not comprise a heterologous sequence encoding a heterologous protein other than a SARS-CoV-2 Omicron variant spike protein or portion thereof (e.g., the ectodomain, S1 domain, S2 domain, or receptor binding domain of SARS-CoV-2 Omicron variant spike protein), or a derivative thereof. In some embodiments, the genome of the recombinant NDV does not comprise a transgene other than a transgene encoding a SARS-CoV-2 Omicron variant spike protein or portion thereof (e.g., the ectodomain, S1 domain, S2 domain, or receptor binding domain of SARS-CoV-2 Omicron variant spike protein), or a derivative thereof. In a specific embodiment, a heterologous sequence encodes a protein that is not found associated with naturally-occurring NDV. In certain embodiments, a recombinant NDV described herein comprises a packaged genome, wherein the genome comprises the genes found in NDV and a transgene encoding a SARS-CoV-2 Omicron variant spike protein or portion thereof (e.g., the ectodomain, S1 domain, S2 domain, or receptor binding domain of SARS-CoV-2 Omicron variant spike protein), or a derivative thereof. In other words, the recombinant NDV encodes for both NDV F protein and the SARS-CoV-2 Omicron variant spike protein or portion thereof (e.g., the ectodomain, S1 domain, S2 domain, or receptor binding domain of SARS-CoV-2 Omicron variant spike protein), or derivative thereof. In some embodiments, a recombinant NDV described herein comprises a packaged genome, wherein the genome comprises the genes found in NDV and a transgene encoding a SARS-CoV-2 Omicron variant spike protein or portion thereof (e.g., the ectodomain, S1 domain, S2 domain or receptor binding domain of SARS-CoV-2 Omicron variant spike protein), or a derivative thereof but does not include any other transgenes.

In some embodiments, the packaged genome of recombinant NDV encodes a chimeric F protein described herein. In certain embodiment, the genome of the recombinant NDV does not comprise a heterologous sequence encoding a heterologous protein other than the chimeric F protein. In a specific embodiment, a heterologous sequence encodes a protein that is not found associated with naturally-occurring NDV. In some embodiments, the genome of the recombinant NDV does not comprise a transgene other than a transgene encoding a chimeric F protein described herein. In preferred embodiments, a recombinant NDV described herein comprises a packaged genome, wherein the genome comprises the genes found in NDV and a transgene encoding a chimeric F protein. In other words, the recombinant NDV encodes for both NDV F protein and the chimeric F protein. In some embodiments, a recombinant NDV described herein comprises a packaged genome, wherein the genome comprises the genes found in NDV and a transgene encoding a chimeric F protein, but does not include any other transgenes.

In a specific embodiment, provided herein is a NDV virion comprising a protein comprising (or consisting of) a SARS-CoV-2 Omicron variant spike protein or a portion thereof (e.g., ectodomain, S1 domain, S2 domain, or receptor binding domain thereof) described herein (e.g., a SARS-CoV-2 Omicron variant spike protein or portion thereof encoded by a transgene described herein), or a derivative thereof. See, e.g., Section 5.1.2 for examples of such a protein that may incorporated into the virion of a recombinant NDV. In a specific embodiment, the protein is one described in Section 5.1.2, supra. In specific embodiments, the NDV virion is recombinantly produced.

In a specific embodiment, provided herein is a NDV virion comprising a protein comprising (or consisting of) a derivative of a SARS-CoV-2 Omicron variant spike protein or a portion thereof (e.g., ectodomain, S1 domain, S2 domain, or receptor binding domain thereof) described herein. See, e.g., Section 5.1.2 for examples of such a protein that may incorporated into the virion of a recombinant NDV. In a specific embodiment, the protein is one described in Section 5.1.2, supra. In specific embodiments, the NDV virion is recombinantly produced.

In a specific embodiment, provided herein is a NDV virion comprising a chimeric F protein described herein (e.g., a chimeric F protein encoded by a transgene described herein). See, e.g., Section 5.1.2 and the Example (e.g., Section 6) for examples of a chimeric F protein that may incorporated into the virion of a recombinant NDV. In a specific embodiment, the chimeric F protein comprises an amino acid sequence that is at least 80%, at least 85%, or at least 90% identical to the amino acid sequence of SEQ ID NO: 8, 9, 12, 13, 16, 17, 31, 37, 43, 49, 55, 61, 67, 75, 81, 87, 93, or 94. In a specific embodiment, the chimeric F protein comprises an amino acid sequence that is at least 95%, at least 98%, or at least 99% identical to the amino acid sequence of SEQ ID NO: 8, 9, 12, 13, 16, 17, 31, 37, 43, 49, 55, 61, 67, 75, 81, 87, 93, or 94. In a specific embodiment, the chimeric F protein comprises the amino acid sequence of SEQ ID NO: 8, 9, 12, 13, 16, 17, 31, 37, 43, 49, 55, 61, 67, 75, 81, 87, 93, or 94. In a specific embodiment, the chimeric F protein is one described in Section 5.1.2 or 6. In specific embodiments, the NDV virion is recombinantly produced.

In a specific embodiment, provided herein is a NDV virion comprising a chimeric F protein described in Section 5.1.2 or 6.

In a specific embodiment, a chimeric F protein described herein is in a pre-fusion conformation. In some embodiments, a chimeric F protein described herein is in a post-fusion conformation.

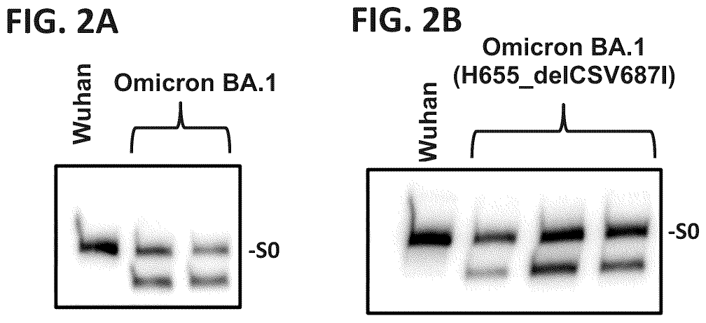

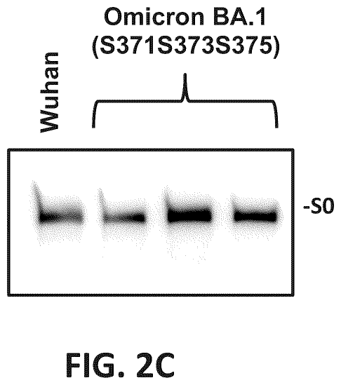

As shown in FIGS. 2A, 2B, 3, 4A, 5A, 5B, 5C, and 7A, certain chimeric F protein expressed by recombinant NDV described in Section 6 undergo proteolysis. Also, as shown in FIG. 2C, 4B, 5B, 6A, 6C, 7B, or 7C, proteolysis of the certain chimeric F protein expressed by certain recombinant NDV described in Section 6 prevented. In specific embodiments, a chimeric F protein described herein does not undergo proteolysis such as shown in FIG. 2A, 2B, 3, 4A, 5A, 5B, 5C, or 7A. In some embodiments, a chimeric F protein described herein that does not undergo proteolysis such as shown in FIG. 2A, 2B, 3, 4A, 5A, 5B, 5C, or 7A, maintains a conformation similar to the ectodomain of the spike protein of a SARS-CoV-2 Omicron variant. In some embodiments, a chimeric F protein described herein that does not undergo proteolysis such as shown in FIG. 2A, 2B, 3, 4A, 5A, 5B, 5C, or 7A yields antibodies that bind to the spike protein of a SARS-CoV-2 Omicron variant in an immunoassay. In some embodiments, a chimeric F protein described herein that does not undergo proteolysis such as shown in FIG. 2A, 2B, 3, 4A, 5A, 5B, 5C, or 7A induces antibodies in a subject that neutralize a SARS-CoV-2 Omicron variant.

### 5.1.4 Codon Optimization

Any codon optimization technique known to one of skill in the art may be used to codon optimize a nucleic acid sequence encoding a SARS-CoV-2 Omicron variant spike protein or a portion thereof (e.g., ectodomain, S1 domain, S2 domain, or receptor binding domain thereof), a derivative of a SARS-CoV-2 Omicron variant spike protein or a portion thereof (e.g., ectodomain, S1 domain, S2 domain, or receptor binding domain thereof), or a chimeric F protein. Methods of codon optimization are known in the art, e.g., the OptimumGene™ (GenScript®) protocol and Genewiz® protocol, which are incorporated by reference herein in its entirety. See also U.S. Pat. No. 8,326,547 for methods for codon optimization, which is incorporated herein by reference in its entirety.

As an exemplary method for codon optimization, each codon in the open frame of the nucleic acid sequence encoding a SARS-CoV-2 Omicron variant spike protein or a portion thereof (e.g., the ectodomain, S1 domain, S2 domain, or receptor binding domain thereof), a derivative of a SARS-CoV-2 Omicron variant spike protein or a portion thereof (e.g., ectodomain, S1 domain, S2 domain, or receptor binding domain thereof), or a chimeric F protein is replaced by the codon most frequently used in mammalian proteins. This may be done using a web-based program (www.encorbio.com/protocols/Codon.htm) that uses the Codon Usage Database, maintained by the Department of Plant Gene Research in Kazusa, Japan. This nucleic acid sequence optimized for mammalian expression may be inspected for: (1) the presence of stretches of 5xA or more that may act as transcription terminators; (2) the presence of restriction sites that may interfere with subcloning; (3) compliance with the rule of six. Following inspection, (1) stretches of 5xA or more that may act as transcription terminators may be replaced by synonymous mutations; (2) restriction sites that may interfere with subcloning may be replaced by synonymous mutations; (3) NDV regulatory signals (gene end, intergenic and gene start sequences), and Kozak sequences for optimal protein expression may be added; and (4) nucleotides may be added in the non-coding region to ensure compliance with the rule of six. Synonymous mutations are typically nucleotide changes that do not change the amino acid encoded. For example, in the case of a stretch of 6 As (AAAAAA), which sequence encodes Lys-Lys, a synonymous sequence would be AAGAAG, which sequence also encodes Lys-Lys.

### 5.2 Construction of NDVS

The recombinant NDVs described herein (see, e.g., Sections 5.1 and 6) can be generated using the reverse genetics technique. The reverse genetics technique involves the preparation of synthetic recombinant viral RNAs that contain the non-coding regions of the negative-strand, viral RNA which are essential for the recognition by viral polymerases and for packaging signals necessary to generate a mature virion. The recombinant RNAs are synthesized from a recombinant DNA template and reconstituted in vitro with purified viral polymerase complex to form recombinant ribonucleoproteins (RNPs) which can be used to transfect cells. A more efficient transfection is achieved if the viral polymerase proteins are present during transcription of the synthetic RNAs either in vitro or in vivo. The synthetic recombinant RNPs can be rescued into infectious virus particles. The foregoing techniques are described in U.S. Pat. No. 5,166,057 issued Nov. 24, 1992; in U.S. Pat. No. 5,854,037 issued Dec. 29, 1998; in U.S. Pat. No. 6,146,642 issued Nov. 14, 2000; in European Patent Publication EP 0702085A1, published Feb. 20, 1996; in U.S. patent application Ser. No. 09/152,845; in International Patent Publications PCT WO 97/12032 published Apr. 3, 1997; WO 96/34625 published Nov. 7, 1996; in European Patent Publication EP A780475; WO 99/02657 published Jan. 21, 1999; WO 98/53078 published Nov. 26, 1998; WO 98/02530 published Jan. 22, 1998; WO 99/15672 published Apr. 1, 1999; WO 98/13501 published Apr. 2, 1998; WO 97/06270 published Feb. 20, 1997; and EPO 780 475A1 published Jun. 25, 1997, each of which is incorporated by reference herein in its entirety.

The helper-free plasmid technology can also be utilized to engineer a NDV described herein. Briefly, a complete cDNA of a NDV (e.g., the Hitchner B1 strain or LaSota strain) is constructed, inserted into a plasmid vector and engineered to contain a unique restriction site between two transcription units (e.g., the NDV P and M genes; the NDV NP and P genes; or the NDV HN and L genes). A nucleotide sequence encoding a heterologous amino acid sequence (e.g., a transgene or other sequence described herein such as, e.g., a nucleotide sequence encoding a SARS-CoV-2 Omicron variant spike protein or portion thereof (e.g., ectodomain or receptor binding domain of the SARS-CoV-2 Omicron variant spike protein), a derivative of a SARS-CoV-2 Omicron variant spike protein or a portion thereof (e.g., ectodomain, S1 domain, S2 domain, or receptor binding domain thereof), or a chimeric F protein) may be inserted into the viral genome at the unique restriction site. Alternatively, a nucleotide sequence encoding a heterologous amino acid sequence (e.g., a transgene or other sequence described herein such as, e.g., a nucleotide sequence encoding SARS-CoV-2 Omicron variant spike protein or portion thereof (e.g., ectodomain or receptor binding domain of the SARS-CoV-2 Omicron variant spike protein), a derivative of a SARS-CoV-2 Omicron variant spike protein or a portion thereof (e.g., ectodomain, S1 domain, S2 domain, or receptor binding domain thereof), or a chimeric F protein) may be engineered into a NDV transcription unit so long as the insertion does not affect the ability of the virus to infect and replicate. The single segment is positioned between a T7 promoter and the hepatitis delta virus ribozyme to produce an exact negative or positive transcript from the T7 polymerase. The plasmid vector and expression vectors comprising the necessary viral proteins are transfected into cells leading to production of recombinant viral particles (see, e.g., International Publication No. WO 01/04333; U.S. Pat. Nos. 7,442,379, 6,146,642, 6,649,372, 6,544,785 and 7,384,774; Swayne et al. (2003). Avian Dis. 47:1047-1050; and Swayne et al. (2001). J. Virol. 11868-11873, each of which is incorporated by reference in its entirety).

Bicistronic techniques to produce multiple proteins from a single mRNA are known to one of skill in the art. Bicistronic techniques allow the engineering of coding sequences of multiple proteins into a single mRNA through the use of IRES sequences. IRES sequences direct the internal recruitment of ribosomes to the RNA molecule and allow downstream translation in a cap independent manner. Briefly, a coding region of one protein is inserted downstream of the ORF of a second protein. The insertion is flanked by an IRES and any untranslated signal sequences necessary for proper expression and/or function. The insertion must not disrupt the open reading frame, polyadenylation or transcriptional promoters of the second protein (see, e.g., Garcia-Sastre et al., 1994, J. Virol. 68:6254-6261 and Garcia-Sastre et al., 1994 Dev. Biol. Stand. 82:237-246, each of which are incorporated by reference herein in their entirety).

Methods for cloning recombinant NDV to encode a transgene and express a heterologous protein encoded by the transgene (e.g., a transgene comprises a nucleotide sequence encoding SARS-CoV-2 Omicron variant spike protein or portion thereof (e.g., ectodomain, S1 domain, S2 domain, or receptor binding domain of the SARS-CoV-2 Omicron variant spike protein), or a derivative thereof, a derivative of a SARS-CoV-2 Omicron variant spike protein or a portion thereof (e.g., ectodomain, S1 domain, S2 domain, or receptor binding domain thereof), or a chimeric F protein) are known to one skilled in the art, such as, e.g., insertion of the transgene into a restriction site that has been engineered into the NDV genome, inclusion an appropriate signals in the transgene for recognition by the NDV RNA-dependent-RNA polymerase (e.g., sequences upstream of the open reading frame of the transgene that allow for the NDV polymerase to recognize the end of the previous gene and the beginning of the transgene, which may be, e.g., spaced by a single nucleotide intergenic sequence), inclusion of a valid Kozak sequence (e.g., to improve eukaryotic ribosomal translation); incorporation of a transgene that satisfies the “rule of six” for NDV cloning; and inclusion of silent mutations to remove extraneous gene end and/or gene start sequences within the transgene. See, e.g., SEQ ID NO:25-28 for examples of a restriction site sequence, gene end sequence, gene start sequence, and Kozak sequence. Regarding the rule of six, one skilled in the art will understand that efficient replication of NDV (and more generally, most members of the paramyxoviridae family) is dependent on the genome length being a multiple of six, known as the “rule of six” (see, e.g., Calain, P. & Roux, L. The rule of six, a basic feature of efficient replication of Sendai virus defective interfering RNA. J. Virol. 67, 4822-4830 (1993)). Thus, when constructing a recombinant NDV described herein, care should be taken to satisfy the “Rule of Six” for NDV cloning. Methods known to one skilled in the art to satisfy the Rule of Six for NDV cloning may be used, such as, e.g., addition of nucleotides downstream of the transgene. See, e.g., Ayllon et al., Rescue of Recombinant Newcastle Disease Virus from cDNA. J. Vis. Exp. (80), e50830, doi: 10.3791/50830 (2013) for a discussion of methods for cloning and rescuing of NDV (e.g., recombinant NDV), which is incorporated by reference herein in its entirety.

In a specific embodiment, an NDV described herein (see, e.g., Section 5.1, and 6) may be generated according to a method described in Section 6, infra.

In a specific embodiment, a recombinant NDV comprising a packaged genome comprising a transgene that comprises a nucleotide sequence encoding a SARS-CoV-2 Omicron variant spike protein or portion thereof (e.g., ectodomain or receptor binding domain of the SARS-CoV-2 Omicron variant spike protein) described herein comprises a LaSota strain backbone. In another specific embodiment, a recombinant NDV comprising a packaged genome comprising a transgene that comprises a nucleotide sequence encoding a SARS-CoV-2 Omicron variant spike protein or portion thereof (e.g., ectodomain, S1 domain, S2 domain, or receptor binding domain of the SARS-CoV-2 Omicron variant spike protein) described herein comprises a LaSota strain backbone. In a specific embodiment, the genomic sequence of the LaSota strain backbone (i.e., without the transgene) is as set forth in SEQ ID NO: 1. In a specific embodiment, the genomic sequence of the LaSota strain backbone (i.e., without the transgene) is as set forth in SEQ ID NO:3. As the skilled person will appreciate, the genome of NDV is negative-sense and single stranded. SEQ ID NOS: 1 and 3 provide cDNA sequences.

In a specific embodiment, a recombinant NDV comprising a packaged genome comprising a transgene that comprises a nucleotide sequence encoding a derivative of a SARS-CoV-2 Omicron variant spike protein or portion thereof (e.g., ectodomain or receptor binding domain of the SARS-CoV-2 Omicron variant spike protein) described herein comprises a LaSota strain backbone. In another specific embodiment, a recombinant NDV comprising a packaged genome comprising a transgene that comprises a nucleotide sequence encoding a derivative of a SARS-CoV-2 Omicron variant spike protein or portion thereof (e.g., ectodomain, S1 domain, S2 domain, or receptor binding domain of the SARS-CoV-2 Omicron variant spike protein) described herein comprises a LaSota strain backbone. In a specific embodiment, the genomic sequence of the LaSota strain backbone (i.e., without the transgene) is as set forth in SEQ ID NO:1. In a specific embodiment, the genomic sequence of the LaSota strain backbone (i.e., without the transgene) is as set forth in SEQ ID NO:3. As the skilled person will appreciate, the genome of NDV is negative-sense and single stranded. SEQ ID NOS: 1 and 3 provide cDNA sequences.

In a specific embodiment, a recombinant NDV comprising a packaged genome comprising a transgene encoding a chimeric F protein described herein comprises a LaSota strain backbone. In a specific embodiment, a recombinant NDV comprising a packaged genome comprising a transgene encoding a chimeric F protein described herein comprises a LaSota strain backbone. In a specific embodiment, the genomic sequence of the LaSota strain backbone (i.e., without the transgene) is as set forth in SEQ ID NO:1. In another specific embodiment, the genomic sequence of the LaSota strain backbone (i.e., without the transgene) is as set forth in SEQ ID NO:3. As the skilled person will appreciate, the genome of NDV is negative-sense and single stranded. SEQ ID NOS: 1 and 3 provide cDNA sequences.

Techniques and procedures described or referenced herein include those that are generally well understood and/or commonly employed using conventional methodology by those skilled in the art, such as, for example, the widely utilized methodologies described in, e.g., Sambrook et al., Molecular Cloning: A Laboratory Manual (3d ed. 2001); Current Protocols in Molecular Biology (Ausubel et al. eds., 2003). Conventional methodologies well understood and/or commonly employed by those of skill in the art may be used to produced a protein described herein.

### 5.3 Propagation of NDVS

The recombinant NDVs described herein (e.g., Sections 5.1 and 6) can be propagated in any substrate that allows the virus to grow to titers that permit the uses of the viruses described herein. In one embodiment, the substrate allows the recombinant NDVs described herein to grow to titers comparable to those determined for the corresponding wild-type viruses.

The recombinant NDVs described herein (e.g., Sections 5.1 and 6) may be grown in cells (e.g., avian cells, chicken cells, etc.) that are susceptible to infection by the viruses, embryonated eggs (e.g., chicken eggs or quail eggs) or animals (e.g., birds). Such methods are well known to those skilled in the art. In a specific embodiment, the recombinant NDVs described herein may be propagated in cancer cells, e.g., carcinoma cells (e.g., breast cancer cells and prostate cancer cells), sarcoma cells, leukemia cells, lymphoma cells, and germ cell tumor cells (e.g., testicular cancer cells and ovarian cancer cells). In another specific embodiment, the recombinant NDVs described herein may be propagated in cell lines, e.g., cancer cell lines such as HeLa cells, MCF7 cells, THP-1 cells, U87 cells, DU145 cells, Lncap cells, and T47D cells. In certain embodiments, the cells or cell lines (e.g., cancer cells or cancer cell lines) are obtained, derived, or obtained and derived from a human(s). In another embodiment, the recombinant NDVs described herein are propagated in interferon deficient systems or interferon (IFN) deficient substrates, such as, e.g., IFN deficient cells (e.g., IFN deficient cell lines) or IFN deficient embryonated eggs. In another embodiment, the recombinant NDVs described herein are propagated in chicken cells or embryonated chicken eggs. Representative chicken cells include, but are not limited to, chicken embryo fibroblasts and chicken embryo kidney cells. In a specific embodiment, the recombinant NDVs described herein are propagated in Vero cells. In another specific embodiment, the recombinant NDVs described herein are propagated in chicken eggs or quail eggs. In certain embodiments, a recombinant NDV virus described herein is first propagated in embryonated eggs and then propagated in cells (e.g., a cell line). In another specific embodiment, the recombinant NDVs described herein are propagated as described in Section 6, infra.

The recombinant NDVs described herein may be propagated in embryonated eggs (e.g. chicken embryonated eggs), e.g., from 6 to 14 days old, 6 to 12 days old, 6 to 10 days old, 6 to 9 days old, 6 to 8 days old, 8 to 10 day old, 9 to 11 days old, or 10 to 12 days old. In a specific embodiment, 10 day old embryonated chicken eggs are used to propagate the recombinant NDVs described herein. Young or immature embryonated eggs (e.g. chicken embryonated eggs) can be used to propagate the recombinant NDVs described herein. Immature embryonated eggs encompass eggs which are less than ten day old eggs, e.g., eggs 6 to 9 days old or 6 to 8 days old that are IFN-deficient. Immature embryonated eggs also encompass eggs which artificially mimic immature eggs up to, but less than ten day old, as a result of alterations to the growth conditions, e.g., changes in incubation temperatures; treating with drugs; or any other alteration which results in an egg with a retarded development, such that the IFN system is not fully developed as compared with ten to twelve day old eggs. The recombinant NDVs described herein can be propagated in different locations of the embryonated egg, e.g., the allantoic cavity (such as, e.g., the allantoic cavity of chicken embryonated eggs). For a detailed discussion on the growth and propagation viruses, see, e.g., U.S. Pat. Nos. 6,852,522 and 7,494,808, both of which are hereby incorporated by reference in their entireties.

In a specific embodiment, a virus is propagated as described in the Example below (e.g., Section 6).

For virus isolation, the recombinant NDVs described herein can be removed from embryonated eggs or cell culture and separated from cellular components, typically by well-known clarification procedures, e.g., such as centrifugation, depth filtration, and microfiltration, and may be further purified as desired using procedures well known to those skilled in the art, e.g., tangential flow filtration (TFF), density gradient centrifugation, differential extraction, or chromatography.

In a specific embodiment, virus isolation from allantoic fluid of an infected egg (e.g., a chicken egg) begins with harvesting allantoic fluid, which is clarified using a filtration system to remove cells and other large debris.

In a specific embodiment, provided herein is a cell (e.g., a cell line) or embryonated egg (e.g., a chicken embryonated egg) comprising a recombinant NDV described herein. In another specific embodiment, provided herein is a method for propagating a recombinant NDV described herein, the method comprising culturing a cell (e.g., a cell line) or embryonated egg (e.g., a chicken embryonated egg) infected with the recombinant NDV. In some embodiments, the method may further comprise isolating or purifying the recombinant NDV from the cell or embryonated egg. In a specific embodiment, provided herein is a method for propagating a recombinant NDV described herein, the method comprising (a) culturing a cell (e.g., a cell line) or embryonated egg infected with a recombinant NDV described herein; and (b) isolating the recombinant NDV from the cell or embryonated egg. The cell or embryonated egg may be one described herein or known to one of skill in the art. In some embodiments, the cell or embryonated egg is IFN deficient. The cell may be one described herein. In specific embodiments, the cell is in vitro or ex vivo. In specific embodiments, the cell(s) is isolated.

In a specific embodiment, provided herein is a method for producing a pharmaceutical composition (e.g., an immunogenic composition) comprising a recombinant NDV described herein, the method comprising (a) propagating a recombinant NDV described herein a cell (e.g., a cell line) or embryonated egg; and (b) isolating the recombinant NDV from the cell or embryonated egg. The method may further comprise adding the recombinant NDV to a container along with a pharmaceutically acceptable carrier.

In some embodiments, provided herein are cells (e.g., cell line) comprising a transgene described herein, polynucleotide described herein, nucleic acid sequence described herein, vector described herein, or nucleotide sequence described herein. The cells may be transfected, transformed, or transduced with the transgene described herein, polynucleotide described herein, nucleic acid sequence described herein, vector described herein, or nucleotide sequence described herein. In some embodiments, provided herein are cells (e.g., cell line) expressing a protein (e.g., a chimeric F protein) described herein. In specific embodiments, the cells are isolated. The cell(s) may be one described herein. In specific embodiments, the cell(s) is in vitro or ex vivo.

### 5.4 Compositions and Routes of Administration

Provided herein are compositions comprising a recombinant NDV described herein (e.g., Section 5.1, or 6). In a specific embodiment, the compositions are pharmaceutical compositions, such as immunogenic compositions (e.g., vaccine compositions). In some embodiments provided herein are compositions (e.g., immunogenic compositions) comprising a transgene described herein, a polynucleotide described herein, a nucleotide sequence described herein, a vector described herein, or a recombinant protein described herein (e.g., Section 5.1, or 6). In some embodiments provided herein are compositions (e.g., immunogenic compositions) comprising a transgene described herein, a polynucleotide described herein, or nucleotide sequence described herein. In some embodiments provided herein are compositions (e.g., immunogenic compositions) comprising a vector described herein. In some embodiments provided herein are compositions (e.g., immunogenic compositions) comprising a recombinant protein described herein (e.g., Section 5.1, or 6). In a specific embodiment, provided herein are immunogenic compositions comprising a recombinant NDV described herein (e.g., Section 5.1, or 6). The compositions may be include a carrier or excipient. For example, the compositions may comprise a pharmaceutically acceptable carrier. The compositions may include an adjuvant (e.g., an adjuvant described herein) or be administered in combination with an adjuvant. The compositions may be used in methods of inducing an immune response to SARS-CoV-2 spike protein. The compositions may or may not include one or more additional prophylactic or therapeutic agents. The compositions may be used in methods for inducing an immune response to SARS-CoV-2 Omicron variant or immunizing against SARS-CoV-2. The compositions may be used in methods for immunizing against COVID-19. The compositions may be used in methods for preventing COVID-19, such as, e.g., preventing severe or moderate COVID-19.

In one embodiment, an immunogenic composition comprises a recombinant NDV described herein (e.g., Section 5.1, or 6), in an admixture with a pharmaceutically acceptable carrier. In some embodiments, the immunogenic composition further comprises one or more additional prophylactic or therapeutic agents. In a specific embodiment, an immunogenic composition comprises an effective amount of a recombinant NDV described herein (e.g., Section 5.1, or 6), and optionally one or more additional prophylactic or therapeutic agents, in a pharmaceutically acceptable carrier. In some embodiments, the recombinant NDV (e.g., Section 5.1, or 6) is the only active ingredient included in the immunogenic composition. In some embodiments, the immunogenic composition is bivalent or multivalent. In some embodiments, the immunogenic composition is monovalent. In a particular embodiment, the immunogenic composition is a vaccine.

In a specific embodiment, administration of an immunogenic composition described herein to a subject (e.g., a human) generates neutralizing antibody (e.g., anti-SARS-CoV-2 spike protein IgG). In certain embodiments, administration of an immunogenic composition described herein to a subject (e.g., a human) generates an immune response that provides some level of protection against developing COVID-19.

In a specific embodiment, the recombinant NDV included in an immunogenic composition described herein is a live virus. In particular, embodiment, the recombinant NDV included in a pharmaceutical composition described herein is an attenuated live virus. In some embodiments, the recombinant NDV included in an immunogenic composition described herein is inactivated. Any technique known to one of skill in the art may be used to inactivate a recombinant NDV described herein. For example, formalin or beta-propiolactone may be used to inactivate a recombinant NDV described herein. In a specific embodiment, the recombinant NDV included in a composition described herein is inactivated using 0.05% to 2% (e.g., 0.05%, 0.1%, 0.5%, 1%, or 2%) beta-Propiolactone, or another technique known to one of skill in the art. For example, in certain embodiments, to prepare inactivated concentrated recombinant NDV, 1 part of 0.5 M disodium phosphate (DSP) may be mixed with 38 parts of the allantoic fluid of an embryonated egg infected with the virus to stabilize the pH, one part of 2% beta-Propiolactone (BPL) is added dropwise to the mixture during shaking, and incubated on ice for 30 min, the mixture is then placed in a 37° C. water bath for approximately 1 to 3 hours shaken every 5-30 min. In another example, recombinant NDV in allantoic fluid is inactivated in 0.05% beta-propiolactone. The inactivated allantoic fluid may be clarified by centrifugation at 4,000 rpm for 20-40 minutes (e.g., about 30 minutes). The clarified allantoic fluids may be laid on top of a 20% sucrose cushion in PBS and ultracentrifuged at 25,000 rpm for about 2 hours at 4° C. using, e.g., a Beckman L7-65 ultracentrifuge with a Beckman SW28 rotor, to pellet the virus through the sucrose cushion to remove soluble egg protein. The virus may then be resuspended in PBS at, e.g., about pH 7 to about 7.6 (such as, e.g., pH 7.4). In specific embodiments, the total protein is determined using the bicinchoninic acid (BCA) assay, or another assay known to one of skill in the art. In a specific embodiment, a chimeric F protein is stable in an inactivated recombinant NDV described herein for a period of time (e.g., for 1 month, 2 months, 3 months, 4 months, 5 months, 6 months, or longer), as assessed by the ability of the inactivated recombinant NDV to induce anti-SARS-CoV-2 spike protein antibodies.

In specific embodiments, an immunogenic composition described herein or a recombinant NDV described herein does not require frozen storage, which makes it difficult to transport and store in low-income countries. In specific embodiments, an immunogenic composition described herein or a recombinant NDV described herein may be stored at about 2° C. to about 8° C. (e.g., 4° C.).

The immunogenic compositions provided herein can be in any form that allows for the composition to be administered to a subject. In a specific embodiment, the pharmaceutical compositions are suitable for veterinary administration, human administration, or both. As used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. The formulation should suit the mode of administration.

In a specific embodiment, the immunogenic compositions are formulated to be suitable for the intended route of administration to a subject. For example, an immunogenic composition may be formulated to be suitable for parenteral, intravenous, intraarterial, intrapleural, inhalation, intranasal, intraperitoneal, oral, intradermal, colorectal, intraperitoneal, and intracranial administration. In one embodiment, an immunogenic composition may be formulated for intravenous, intraarterial, oral, intraperitoneal, intranasal, intratracheal, intrapleural, intracranial, subcutaneous, intramuscular, topical, or pulmonary administration. In a specific embodiment, an immunogenic composition may be formulated for intranasal administration. In certain embodiments, an immunogenic composition is formulated for a nasal spray. In another embodiment, an immunogenic composition may be formulated for intramuscular administration.

In a specific embodiment, an immunogenic composition comprising a recombinant NDV described herein (see, e.g., Sections 5.1 and 6) is formulated to be suitable for intranasal administration to the subject (e.g., human subject).

In a specific embodiment, an immunogenic composition comprising an inactivated recombinant NDV described herein may comprise an adjuvant. In some embodiments, an immunogenic composition comprising a polynucleotide described herein, nucleotide sequence described herein, a vector described herein, or a recombinant protein described herein may comprise an adjuvant. In certain embodiments, the compositions described herein comprise, or are administered in combination with, an adjuvant. The adjuvant for administration in combination with a composition described herein may be administered before, concomitantly with, or after administration of the composition. In specific embodiments, an inactivated virus immunogenic composition described herein comprises one or more adjuvants. In some embodiments, the term “adjuvant” refers to a compound that when administered in conjunction with or as part of a composition described herein augments, enhances and/or boosts the immune response to a recombinant NDV, but when the compound is administered alone does not generate an immune response to the virus. In some embodiments, the adjuvant generates an immune response to a recombinant NDV and does not produce an allergy or other adverse reaction. Adjuvants can enhance an immune response by several mechanisms including, e.g., lymphocyte recruitment, stimulation of B and/or T cells, and stimulation of macrophages. Specific examples of adjuvants include, but are not limited to, aluminum salts (alum) (such as aluminum hydroxide, aluminum phosphate, and aluminum sulfate), 3 De-O-acylated monophosphoryl lipid A (MPL) (see GB 2220211), MF59 (Novartis), AS03 (GlaxoSmithKline), AS04 (GlaxoSmithKline), polysorbate 80 (Tween 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al., in Vaccine Design: The Subunit and Adjuvant Approach (eds. Powell & Newman, Plenum Press, NY, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al, N. Engl. J. Med. 336, 86-91 (1997)). Another adjuvant is CpG (Bioworld Today, Nov. 15, 1998). Such adjuvants can be used with or without other specific immunostimulating agents such as MPL or 3-DMP, QS21, polymeric or monomeric amino acids such as poly glutamic acid or polylysine. In certain embodiments, the adjuvant is a liposomal suspension adjuvant (R-enantiomer of the cationic lipid DOTAP, R-DOTAP) or an MF-59 like oil-in-water emulsion adjuvant (AddaVax). The adjuvant may be a toll-like receptor (TLR) agonist (e.g., a TLR7 agonist, TLR8 agonist, TLR7/8 agonist, or TLR9 agonist). In some embodiments, the adjuvant is a toll-like receptor 9 (TLR9) agonist adjuvant. In certain embodiments, the adjuvant is CpG 1018. In some embodiments, a composition described herein (e.g., a live recombinant NDV composition) does not contain an adjuvant.

In certain embodiments, an immunogenic composition described herein comprises an effective amount of a recombinant NDV described herein. In specific embodiments, an effective amount of a recombinant NDV described herein is an amount of recombinant NDV to generate an immune response in a subject or a population of subjects. In specific embodiments, an effective amount of a recombinant NDV described herein is 10⁴ to 10¹² PFU or EID50. In some embodiments, an effective amount comprises 1 to 15 micrograms of a recombinant protein described herein. In some embodiments, an effective amount comprises 1 to 15 micrograms of a SARS-CoV-2 spike protein or a portion thereof (e.g., an ectodomain), a derivative of a SARS-CoV-2 spike protein or a portion thereof (e.g., an ectodomain), or a chimeric F protein expressed by a recombinant NDV described herein.

In certain embodiments, an immunogenic composition described herein comprises 10⁴ to 10¹² EID50 of a recombinant NDV described herein. In some embodiments, an immunogenic composition described herein comprises 1 to 15 micrograms of a SARS-CoV-2 Omicron variant spike protein or a portion thereof (e.g., an ectodomain), a derivative of a SARS-CoV-2 Omicron variant spike protein or a portion thereof (e.g., an ectodomain), or a chimeric F protein expressed by a recombinant NDV described herein. In some embodiments, an immunogenic composition described herein comprises 1 to 15 micrograms of a SARS-CoV-2 Omicron variant spike protein or a portion thereof (e.g., an ectodomain), a derivative of a SARS-CoV-2 Omicron variant spike protein or a portion thereof (e.g., an ectodomain), or a chimeric F protein expressed by a recombinant NDV described herein. In some embodiments, pharmaceutical composition (e.g., an immunogenic composition) described herein comprises 1 to 15 micrograms per ml of a SARS-CoV-2 Omicron variant spike protein or a portion thereof (e.g., an ectodomain), a derivative of a SARS-CoV-2 spike protein or a portion thereof (e.g., an ectodomain), or a chimeric F protein expressed by a recombinant NDV described herein.

In some embodiments, an immunogenic composition described herein comprises 1 to 15 micrograms of inactivated recombinant NDV described herein.

In a specific embodiment, an immunogenic composition described herein may be stored at 2° to 8° C. (e.g., 4° C.). In certain embodiments, an immunogenic composition described herein is stable for at least 1 month, at least 2 months, at least 3 months, at least 4 months, at least 5 months, at least 6 months, at least 9 months or at least 1 year at 2° to 8° C. In some embodiments, an immunogenic composition described herein is stable for 3-6 months, 3-9 months, 6-12 months, or 9-12 months at 2° to 8° C. (e.g., 4° C.). In certain embodiments, the stability is assessed by protein denaturation assays, immunoassays or a combination thereof.

### 5.5 Uses of a Recombinant NDV

### 5.5.1 Prevention of COVID-19

The recombinant NDV(s) described herein or an immunogenic composition described herein may be used to immunize a subject against SARS-CoV-2, induce an immune response to SARS-CoV-2 spike protein, or prevent COVID-19. In a specific aspect, the recombinant NDV(s) described herein may be used to immunize a subject against a SARS-CoV-2 Omicron variant, induce an immune response to a SARS-CoV-2 Omicron variant spike protein, or prevent COVID-19 caused by or associated with a SARS-CoV-2 Omicron variant. In another specific aspect, an immunogenic composition described herein may be used to immunize a subject against a SARS-CoV-2 Omicron variant, induce an immune response to a SARS-CoV-2 Omicron variant spike protein, or prevent COVID-19 caused by or associated with a SARS-CoV-2 Omicron variant.

In one aspect, presented herein are methods for inducing an immune response in a subject (e.g., a human subject), comprising administering the subject (e.g., a human subject) a recombinant NDV described herein or an immunogenic composition comprising a recombinant NDV described herein. In one embodiment, presented herein is a method for inducing an immune response to a SARS-CoV-2 spike protein in a subject (e.g., a human subject), comprising administering the subject (e.g., a human subject) a recombinant NDV described herein or an immunogenic composition described herein, such as described in Section 5.4. In another embodiment, presented herein is a method for inducing an immune response to a SARS-CoV-2 spike protein in a subject (e.g., a human subject), comprising administering the subject (e.g., a human subject) an effective amount of a recombinant NDV described herein or an immunogenic composition described herein. See, e.g., Section 5.1 and 6 for recombinant NDV and Section 5.4 or 6 for immunogenic compositions. In a specific embodiment, the recombinant NDV is one described in Section 5.1 or 6, and the immunogenic composition is one described in Section 5.4 or 6.

In a specific embodiment, presented herein is a method for inducing an immune response to a SARS-CoV-2 Omicron variant spike protein in a subject (e.g., a human subject), comprising administering the subject (e.g., a human subject) a recombinant NDV described herein, or an immunogenic composition described herein. In another specific embodiment, presented herein is a method for inducing an immune response to a SARS-CoV-2 Omicron variant spike protein in a subject (e.g., a human subject), comprising administering the subject (e.g., a human subject) an effective amount of a recombinant NDV described herein, or an immunogenic composition described herein. In a specific embodiment, the immunogenic composition is one described in Section 5.4 or 6.

In another aspect, presented herein are methods for immunizing a subject (e.g., a human subject) against SARS-CoV-2 (e.g., a SARS-CoV-2 Omicron variant) comprising administering the subject (e.g., a human subject) a recombinant NDV described herein or an immunogenic composition comprising a recombinant NDV described herein. In one embodiment, presented herein is a method for immunizing a subject (e.g., a human subject) against SARS-CoV-2 (e.g., a SARS-CoV-2 Omicron variant), comprising administering the subject (e.g., a human subject) a recombinant NDV described herein, or an immunogenic composition described herein. In another embodiment, presented herein is a method for immunizing a subject (e.g., a human subject) against SARS-CoV-2 (e.g., a SARS-CoV-2 Omicron variant), comprising administering the subject (e.g., a human subject) an effective amount of a recombinant NDV described herein, or an immunogenic composition described herein. See, e.g., Section 5.1 and 6 for recombinant NDV and Section 5.4 and 6 for compositions. In a specific embodiment, the recombinant NDV is one described in Section 5.1 or 6, and the immunogenic composition is one described in Section 5.4 or 6.

In another aspect, presented herein are methods for preventing COVID-19 in a subject (e.g., a human subject), comprising administering the subject (e.g., a human subject) a recombinant NDV described herein, or an immunogenic composition comprising a recombinant NDV described herein. In one embodiment, presented herein is a method for preventing COVID-19 in a subject (e.g., a human subject), comprising administering the subject (e.g., a human subject) a recombinant NDV described herein or an immunogenic composition described herein. In another embodiment, presented herein is a method for preventing COVID-19 in a subject (e.g., a human subject), comprising administering the subject (e.g., a human subject) an effective amount of a recombinant NDV described herein or an immunogenic composition described herein. In some embodiments, moderate COVID-19 is prevented. In some embodiments, severe COVID-19 is prevented. In a specific embodiment, the recombinant NDV is one described in Section 5.1 or 6, and the immunogenic composition is one described in Section 5.4 or 6. The COVID-19 may be caused by or associated with a SARS-CoV-2 Omicron variant.

The recombinant NDV described herein may be administered to a subject in combination with one or more other therapies. The recombinant NDV and one or more other therapies may be administered by the same or different routes of administration to the subject. In a specific embodiment, the recombinant NDV is administered to a subject intranasally. See, e.g., Sections 5.1, and 6, infra for information regarding recombinant NDV, Section 5.5.3 for information regarding other therapies, and Section 5.4, infra, for information regarding compositions and routes of administration.

The recombinant NDV and one or more additional therapies may be administered concurrently or sequentially to the subject. In certain embodiments, the recombinant NDV and one or more additional therapies are administered in the same composition. In other embodiments, the recombinant NDV and one or more additional therapies are administered in different compositions. The recombinant NDV and one or more other therapies may be administered by the same or different routes of administration to the subject. Any route known to one of skill in the art or described herein may be used to administer the recombinant NDV and one or more other therapies. In a specific embodiment, the recombinant NDV is administered intranasally or intramuscularly and the one or more other therapies are administered by the same or a different route. In a specific embodiment, the recombinant NDV is administered intranasally and the one or more other therapies is administered intravenously.

In some embodiments, two immunogenic compositions described herein are administered concurrently or sequentially to the subject. In some embodiments, three immunogenic compositions described herein are administered concurrently or sequentially to the subject. In some embodiments, three immunogenic compositions described herein are administered concurrently or sequentially to the subject. In some embodiments, four immunogenic compositions described herein are administered concurrently or sequentially to the subject.

In certain embodiments, a recombinant NDV described herein or an immunogenic composition described herein is administered to a subject previously vaccinated with a COVID-19 vaccine. In some embodiments, a recombinant NDV described herein or an immunogenic composition described herein is administered to a subject previously vaccinated with a COVID-19 vaccine other than a NDV-based COVID-19 vaccine. The COVID-19 vaccine may be a protein subunit vaccine, vector vaccine, or an mRNA vaccine. The COVID-19 vaccine may be Pfizer's COVID-19 vaccine, Pfizer-BioNTech bivalent COVID-19 vaccine, Moderna's COVID-19 vaccine, Moderna's bivalent COVID-19 vaccine, AstraZeneca's COVID-19 vaccine, Johnson & Johnson's COVID-19, Novavax COVID-19 Vaccine, Adjuvanted, SinoVac's COVID-19 vaccine, SinoPharm's COVID-19 vaccine, Bharat's COVID-19 vaccine, Cansino's COVID-19 vaccine, or another COVID-19 vaccine. In certain embodiments, a recombinant NDV described herein or an immunogenic composition described herein is administered to a subject previously vaccinated with an immunogenic composition other than one described herein. In certain embodiments, a recombinant NDV described herein or an immunogenic composition described herein is administered to a subject previously infected with SARS-CoV-2. In some embodiments, a recombinant NDV described herein or an immunogenic composition described herein is administered to a subject previously diagnosed with a SARS-CoV-2 infection. In some embodiments, a recombinant NDV described herein or an immunogenic composition described herein is administered to a subject previously experiencing symptoms of COVID-19. In certain embodiments, a recombinant NDV described herein or an immunogenic composition described herein is administered to a subject previously diagnosed with COVID-19.

In a specific embodiment, the immune response resulting from administration of a recombinant NDV described herein, or an immunogenic composition described herein provides some protection against COVID-19 caused by or associated with a SARS-CoV-2 Omicron variant. In another specific embodiment, an antibody induced by a recombinant NDV described herein, or an immunogenic composition described herein binds to a SARS-CoV-2 spike protein Omicron variant. In another specific embodiment, an antibody induced by a recombinant NDV described herein, or an immunogenic composition described herein may neutralize a SARS-CoV-2 Omicron variant, as assessed by an assay described herein or known to one of skill in the art. In some embodiments, the immune response resulting from administration of a recombinant NDV described herein, or an immunogenic composition described herein provides some protection against COVID-19 caused by or associated a SARS-CoV-2 Omicron variant, as assessed by an assay described herein or known to one of skill in the art.

In some embodiments, a recombinant NDV described herein or an immunogenic composition described herein, or a combination therapy described herein is administered to a patient to prevent the onset of one, two or more symptoms of COVID-19. In a specific embodiment, the administration of a recombinant NDV described herein or an immunogenic composition described herein, or a combination therapy described herein to a subject prevents the onset or development of one, two or more symptoms of COVID-19, or reduces the severity of one, two or more symptoms of COVID-19. In a specific embodiment, the administration of a recombinant NDV described herein or an immunogenic composition described herein, or a combination therapy described herein to a subject prevents the onset or development of one, two or more symptoms of COVID-19 and reduces the severity of one, two or more symptoms of COVID-19. Symptoms of COVID-19 include congested or runny nose, cough, fever, sore throat, fatigue, headache, wheezing, rapid or shallow breathing or difficulty breathing, bluish color the skin due to lack of oxygen, chills, muscle pain, loss of taste and/or smell, nausea, vomiting, and diarrhea.

In one embodiment, the administration of a recombinant NDV described herein, an immunogenic composition described herein, or a combination therapy described herein to a subject prevents the spread of SARS-CoV-2 infection. In a specific embodiment, the administration of a recombinant NDV described herein, an immunogenic composition described herein, or a combination therapy described herein to a subject prevents the spread of SARS-CoV-2 Omicron variant virus infection. In another specific embodiment, the administration of a recombinant NDV described herein, an immunogenic composition described herein, or a combination therapy described herein to a subject prevents hospitalization. In another specific embodiment, the administration of a recombinant NDV described herein or an immunogenic composition described herein, or a combination therapy described herein to a subject prevents COVID-19. In another specific embodiment, the administration of a recombinant NDV described herein or an immunogenic composition described herein, or a combination therapy described herein to a subject prevents moderate or severe COVID-19. In another embodiment, the administration of a recombinant NDV described herein, an immunogenic composition described herein, or a combination therapy described herein to a subject reduces the length of hospitalization. In another embodiment, the administration of a recombinant NDV described herein, an immunogenic composition described herein, or a combination therapy described herein to a subject reduces the likelihood of intubation. In another specific embodiment, the administration of a recombinant NDV described herein, an immunogenic composition described herein, or a combination therapy described herein to a subject prevents recurring SARS-CoV-2 infections. In another specific embodiment, the administration of a recombinant NDV described herein, an immunogenic composition described herein, or a combination therapy described herein to a subject prevents recurring SARS-CoV-2 Omicron virus infections. In another specific embodiment, the administration of a recombinant NDV described herein, an immunogenic composition described herein, or a combination therapy described herein to a subject prevents asymptomatic SARS-CoV-2 infection. In another specific embodiment, the administration of a recombinant NDV described herein, an immunogenic composition described herein, or a combination therapy described herein to a subject prevents asymptomatic SARS-CoV-2 Omicron variant virus infection.

In another specific embodiment, the administration of a recombinant NDV described herein, or an immunogenic composition described herein induces antibodies to SARS-CoV-2 spike protein. In another specific embodiment, the administration of a recombinant NDV described herein, or an immunogenic composition described herein induces antibodies specific to SARS-CoV-2 spike protein. An antibody (ies) may specifically bind to a SARS-CoV-2 Omicron variant spike protein if it binds to the SARS-CoV-2 Omicron variant spike protein with a higher affinity than a spike protein that is not a SARS-CoV-2 Omicron variant spike protein, or other unrelated protein. For example, an antibody (ies) specific for SARS-CoV-2 Omicron variant spike protein may bind to a SARS-CoV-2 Omicron variant spike protein with a 10 fold higher for affinity than the antibody (ies) binds to a spike protein that is not a SARS-CoV-2 Omicron spike protein, or other unrelated protein. In some embodiments, the administration of a recombinant NDV described herein, or an immunogenic composition induces a higher concentration of antibody (ies) that specifically bind to a SARS-CoV-2 Omicron variant spike protein than the administration of a recombinant NDV comprising a chimeric F protein comprising the ectodomain of SEQ ID NO: 104, and the transmembrane and cytoplasmic domains of NDV F protein, such as described in Example 5.

In another specific embodiment, the administration of a recombinant NDV described herein, or an immunogenic composition described herein induces both mucosal and systemic antibodies to SARS-CoV-2 Omicron spike protein (e.g., neutralizing antibodies). In another specific embodiment, the administration of a recombinant NDV described herein, an immunogenic composition described herein, or a combination therapy described herein to a subject induces neutralizing IgG antibody to SARS-CoV-2 Omicron variant spike protein. In another specific embodiment, the administration of a recombinant NDV described herein, an immunogenic composition described herein, or a combination therapy described herein to a subject induces IgG antibody to SARS-CoV-2 Omicron variant spike protein at a level that is considerate moderate to high in an ELISA approved by the FDA for measuring antibody in a patient specimen.

In another specific embodiment, the administration of a recombinant NDV described herein, an immunogenic composition described herein, or a combination therapy described herein to a subject induces neutralizing antibody to SARS-CoV-2 spike protein. In another specific embodiment, the administration of a recombinant NDV described herein, an immunogenic composition described herein, or a combination therapy described herein to a subject induces neutralizing antibody to SARS-CoV-2 Omicron variant spike protein.

In some embodiments, a recombinant NDV described herein or a composition thereof, or a combination therapy described herein is administered to a subject predisposed or susceptible to COVID-19.

In certain embodiments, a recombinant NDV described herein, an immunogenic composition described herein, or a combination therapy described herein is administered to a human. In some embodiments, a recombinant NDV described herein, an immunogenic composition described herein, or a combination therapy described herein is administered to a human infant. In another specific embodiment, the subject is a human infant six months old or older. In other embodiments, a recombinant NDV described herein, an immunogenic composition described herein, or a combination therapy described herein is administered to a human toddler. In other embodiments, a recombinant NDV described herein, an immunogenic composition described herein, or a combination therapy described herein is administered to a human child. In other embodiments, a recombinant NDV described herein, an immunogenic composition described herein, or a combination therapy described herein is administered to a human adult. In yet other embodiments, a recombinant NDV described herein, an immunogenic composition described herein, or a combination therapy described herein is administered to an elderly human.

In a specific embodiment, a recombinant NDV described herein, an immunogenic composition described herein, or a combination therapy described herein is administered a subject (e.g., a human subject) in close contact with an individual with increased risk of COVID-19 or SARS-CoV-2 infection (e.g., a SARS-CoV-2 Omicron variant infection). In some embodiments, a recombinant NDV described herein, an immunogenic composition described herein, or a combination therapy described herein is administered a subject (e.g., a human subject) with a condition that increases susceptibility to SARS-CoV-2 complications or for which SARS-CoV-2 increases complications associated with the condition. Examples of conditions that increase susceptibility to SARS-CoV-2 complications or for which SARS-CoV-2 increases complications associated with the condition include conditions that affect the lung, such as cystic fibrosis, chronic obstructive pulmonary disease (COPD), emphysema, asthma, or bacterial infections (e.g., infections caused by Haemophilus influenzae, Streptococcus pneumoniae, Legionella pneumophila, and Chlamydia trachomatous); cardiovascular disease (e.g., congenital heart disease, congestive heart failure, and coronary artery disease); and endocrine disorders (e.g., diabetes).

In some embodiments, a recombinant NDV described herein, an immunogenic composition described herein, or a combination therapy described herein is administered a subject (e.g., a human subject) that resides in a group home, such as a nursing home. In some embodiments, a recombinant NDV described herein, an immunogenic composition described herein, or a combination therapy described herein is administered a subject (e.g., a human subject) that works in, or spends a significant amount of time in, a group home, e.g., a nursing home. In some embodiments, a recombinant NDV described herein, an immunogenic composition described herein, or a combination therapy described herein is administered a subject (e.g., a human subject) that is a health care worker (e.g., a doctor or nurse). In some embodiments, a recombinant NDV described herein, an immunogenic composition described herein, or a combination therapy described herein is administered a subject (e.g., a human subject) that is a smoker.

In some embodiments, a recombinant NDV described herein, an immunogenic composition described herein, or a combination therapy described herein is administered to: (1) a subject (e.g., a human subject) who can transmit SARS-CoV-2 to those at high risk for complications, such as, e.g., members of households with high-risk subjects, including households that will include human infants (e.g., infants younger than 6 months), (2) a subject coming into contact with human infants (e.g., infants less than 6 months of age), (3) a subject who will come into contact with subjects who live in nursing homes or other long-term care facilities, (4) a subject who is or will come into contact with an elderly human, or (5) a subject who will come into contact with subjects with long-term disorders of the lungs, heart, or circulation; individuals with metabolic diseases (e.g., diabetes) or subjects with weakened immune systems (including immunosuppression caused by medications, malignancies such as cancer, organ transplant, or HIV infection).

### 5.5.2 Dosage and Frequency

The amount of a recombinant NDV or an immunogenic composition described herein, which will be effective in the prevention of COVID-19, or immunization against SARS-CoV-2 (e.g., SARS-CoV-2 Omicron variant) will depend on the route of administration, the general health of the subject, etc. Suitable dosage ranges of a recombinant NDV for administration are generally about 10⁴ to about 10¹² EID50, and can be administered to a subject once, twice, three, four or more times with intervals as often as needed. In some embodiments, a recombinant NDV described herein is administered to a subject (e.g., human) at a dose of 10⁴ to about 10¹² EID50. In some embodiments, a dose of about 10⁴ to about 10¹² EID50 of a composition comprising live recombinant NDV is administered to a subject (e.g., human). In a specific embodiment, a live recombinant NDV described herein is administered to a subject (e.g., human) at a dose of 10⁷ to 10⁹ EID50. In another specific embodiment, a dose of 10⁷ to 10⁹ EID50 of a composition comprising a live recombinant NDV described herein is administered to a subject (e.g., a human). In a specific embodiment, a live recombinant NDV described herein is administered to a subject (e.g., human) at a dose of about 10⁸ to about 10⁹ EID50. In a specific embodiment, a live recombinant NDV described herein is administered to a subject (e.g., human) at a dose of about 10⁷ to about 10⁸ EID50.

In certain embodiments, a recombinant NDV described herein is administered to a subject (e.g., human) at a dose of 1 to 15 micrograms of SARS-CoV-2 Omicron variant spike protein or a portion thereof (e.g., the ectodomain of a SARS-CoV-2 spike protein), a derivative of SARS-CoV-2 Omicron variant spike protein or a portion thereof (e.g., a derivative of the ectodomain of a SARS-CoV-2 Omicron variant spike protein), or a chimeric F protein. In some embodiments, a recombinant NDV described herein is administered to a subject (e.g., human) at a dose of 1 to 10 micrograms of SARS-CoV-Omicron variant spike protein or a portion thereof (e.g., the ectodomain of a SARS-CoV-2 Omicron variant spike protein), a derivative of a SARS-CoV-2 Omicron variant spike protein or a portion thereof (e.g., a derivative of the ectodomain of a SARS-CoV-2 Omicron variant spike protein), or a chimeric F protein. In a specific embodiment, a recombinant NDV described herein is administered to a subject (e.g., human) at a dose of 1 microgram, 3 micrograms, or 10 micrograms of SARS-CoV-2 Omicron variant spike protein or a portion thereof (e.g., the ectodomain of a SARS-CoV-2 Omicron variant spike protein), a derivative of SARS-CoV-2 Omicron variant spike protein or a portion thereof (e.g., a derivative of the ectodomain of a SARS-CoV-2 Omicron variant spike protein), or a chimeric F protein. In another specific embodiment, a recombinant NDV described herein is administered to a subject (e.g., human) at a dose of 4 micrograms, 5 micrograms, 6 micrograms, 7 micrograms, 8 micrograms or 9 micrograms of SARS-CoV-2 Omicron variant spike protein or a portion thereof (e.g., the ectodomain of a SARS-CoV-2 Omicron variant spike protein), a derivative of SARS-CoV-2 Omicron variant spike protein or a portion thereof (e.g., a derivative of the ectodomain of a SARS-CoV-2 Omicron variant spike protein), or a chimeric F protein.

In certain embodiments, a composition described herein is administered to a subject (e.g., human) at a dose of 1 to 15 micrograms of SARS-CoV-2 Omicron variant spike protein or a portion thereof (e.g., the ectodomain of a SARS-CoV-2 Omicron variant spike protein), a derivative of SARS-CoV-2 Omicron variant spike protein or a portion thereof (e.g., a derivative of the ectodomain of a SARS-CoV-2 Omicron variant spike protein), or a chimeric F protein. In some embodiments, an immunogenic composition described herein is administered to a subject (e.g., human) at a dose of 1 to 10 micrograms of SARS-CoV-2 Omicron variant spike protein or a portion thereof (e.g., the ectodomain of a SARS-CoV-2 Omicron variant spike protein), a derivative of SARS-CoV-2 Omicron variant spike protein or a portion thereof (e.g., a derivative of the ectodomain of a SARS-CoV-2 Omicron variant spike protein), or a chimeric F protein. In a specific embodiment, an immunogenic composition NDV described herein is administered to a subject (e.g., human) at a dose of 1 microgram, 3 micrograms, or 10 micrograms of SARS-CoV-2 Omicron variant spike protein or a portion thereof (e.g., the ectodomain of a SARS-CoV-2 Omicron variant spike protein), a derivative of SARS-CoV-2 Omicron variant spike protein or a portion thereof (e.g., a derivative of the ectodomain of a SARS-CoV-2 Omicron variant spike protein), or a chimeric F protein. In another specific embodiment, an immunogenic composition described herein is administered to a subject (e.g., human) at a dose of 4 micrograms, 5 micrograms, 6 micrograms, 7 micrograms, 8 micrograms or 9 micrograms of SARS-CoV-2 Omicron variant spike protein or a portion thereof (e.g., the ectodomain of a SARS-CoV-2 Omicron variant spike protein), a derivative of SARS-CoV-2 Omicron variant spike protein or a portion thereof (e.g., a derivative of the ectodomain of a SARS-CoV-2 Omicron variant spike protein), or a chimeric F protein.

In some embodiments, an immunogenic composition described herein is administered to a subject (e.g., human) at a dose of 10 to 100 micrograms of inactivated recombinant NDV described herein. In some embodiments, an immunogenic composition described herein is administered to a subject (e.g., human) at a dose of 50 to 100 micrograms of inactivated recombinant NDV described herein. In specific embodiments, an immunogenic composition described herein is administered to a subject (e.g., human) at a dose of 10 micrograms, 25 micrograms, 30 micrograms, 50 micrograms, 75 micrograms, or 100 micrograms of inactivated recombinant NDV described herein.

In certain embodiments, dosages of a recombinant NDV described herein, or a composition described herein similar to those currently being used in clinical trials for NDV are administered to a subject.

In certain embodiments, a recombinant NDV or an immunogenic composition described herein is administered to a subject as a single dose followed by a second dose 1 to 6 weeks, 1 to 5 weeks, 1 to 4 weeks, 1 to 3 weeks, 1 to 2 weeks, 6 to 12 weeks, 3 to 6 months, 6 to 9 months, 6 to 12 months, or 6 to 9 months later. In some embodiments, a recombinant NDV or an immunogenic composition described herein is administered to a subject as a single dose followed by a second dose about 3 to about 6 months, about 6 to about 9 months, or about 6 to about 12 months later. In specific embodiments, a recombinant NDV or an immunogenic composition described herein is administered to a subject as a single dose followed by a second dose about 6 months, about 9 months, or about 12 months later. In accordance with these embodiments, booster inoculations may be administered to the subject at 3 to 6 month or 6 to 12 month intervals following the second inoculation. In accordance with these embodiments, booster inoculations may be administered to the subject at about 6 months following the second inoculation. In certain embodiments, a subject is administered one or more boosters. The recombinant NDV used for each booster may be the same or different. The two, three, four, or more recombinant NDVs described herein, or immunogenic compositions described herein administered to the subject may administered by the same or different routes. For example, one recombinant NDV or an immunogenic composition described herein may be administered to the subject intranasally and another recombinant NDV described herein or immunogenic composition described herein may be administered to the subject intramuscularly. In another example, one recombinant NDV herein or an immunogenic composition described herein may be administered to the subject intramuscularly and another recombinant NDV described herein or immunogenic composition described herein may be administered to the subject intranasally. In another example, one recombinant NDV described herein or an immunogenic composition described herein may be administered to the subject intranasally or intramuscularly and another recombinant NDV or immunogenic composition described herein may be administered to the subject by the same route of administration.

In certain embodiments, administration of the same recombinant NDV or immunogenic composition may be repeated and the administrations may be separated by at least 7 days, 10 days, 14 days, 15 days, 21 days, 28 days, 30 days, 45 days, 2 months, 75 days, 3 months, or at least 6 months. In other embodiments, administration of the same recombinant NDV or immunogenic composition may be repeated and the administrations may be separated by 1 to 14 days, 1 to 7 days, 7 to 14 days, 1 to 30 days, 15 to 30 days, 15 to 45 days, 15 to 75 days, 15 to 90 days, 1 to 3 months, 3 to 6 months, 3 to 12 months, or 6 to 12 months. In some embodiments, a first recombinant NDV or immunogenic composition is administered to a subject followed by the administration of a second recombinant NDV or a immunogenic composition. In some embodiments, the first and second recombinant NDV are different from each other. In certain embodiments, a first immunogenic composition is administered to a subject as a priming dose and after a certain period (e.g., 1 month, 2 months, 3 months, 4 months, 5 months, 6 months, 9 months, 12 months, 1-6 months, 6-9 months, or 9-12 months) a booster dose of a second immunogenic composition is administered.

In some embodiments, a regimen, such as described in Example 5, or similar to a regimen described in Example 5, is used to administer an immunogenic composition described herein.

In certain embodiments, a first dose of a recombinant NDV described herein or an immunogenic composition described herein may be administered to a subject (e.g., a human) and a second dose of the recombinant NDV or immunogenic composition may be administered to the subject 3 to 6 weeks later. In some embodiments, a first dose of a recombinant NDV described herein or an immunogenic composition described herein may be administered to a subject (e.g., a human) and a second dose of the recombinant NDV or immunogenic composition may be administered to the subject about 21 days later. In some embodiments, a first dose of a recombinant NDV described herein or immunogenic composition may be administered to a subject (e.g., a human) and a second dose of the recombinant NDV or an immunogenic composition described herein may be administered to the subject about 3-6 months later. In some embodiments, a first dose of a recombinant NDV described herein or an immunogenic composition described herein may be administered to a subject (e.g., a human) and a second dose of the recombinant NDV or immunogenic composition may be administered to the subject about 6-12 months later. In some embodiments, the subject is administered two or more boosters of the recombinant NDV.

In some embodiments, a recombinant NDV described herein or an immunogenic composition described is administered as a booster to a subject previously vaccinated with a COVID-19 vaccine. The COVID-9 vaccine may be an mRNA vaccine, a vector vaccine (e.g., a virus vector vaccine), or a protein subunit-based vaccine. The COVID-19 vaccine may be Pfizer's COVID-19 vaccine (BNT162b2), Pfizer-BioNTech bivalent COVID-19 vaccine, Moderna's COVID-19 vaccine (mRNA-1273), Moderna's bivalent COVID-19 vaccine, AstraZeneca's COVID-19 vaccine, Johnson & Johnson's COVID-19 (Ad26.COV2.S), SinoVac's COVID-19 vaccine, SinoPharm's COVID-19 vaccine, Bharat's COVID-19 vaccine, Novavax COVID-19 Vaccine, Adjuvanted, Cansino's COVID-19 vaccine, or another COVID-19 vaccine. In a specific embodiment, the subject was previously vaccinated with a COVID-19 other than an immunogenic composition described herein. In a specific embodiment, the subject was previously vaccinated with a COVID-19 other than a recombinant NDV-based COVID-19 vaccine.

In some embodiments, a recombinant NDV described herein or an immunogenic composition described herein is administered as a booster to a subject previously infected with SARS-CoV-2. In certain embodiments, a recombinant NDV described herein or an immunogenic composition described herein is administered as a booster to a subject previously diagnosed with a SARS-CoV-2 infection.

In certain embodiments, a recombinant NDV or an immunogenic composition described herein is administered to a subject in combination with one or more additional therapies, such as a therapy described in Section 5.5.3, infra. The dosage of the other one or more additional therapies will depend upon various factors including, e.g., the therapy, the route of administration, the general health of the subject, etc. and should be decided according to the judgment of a medical practitioner. In specific embodiments, the dose of the other therapy is the dose and/or frequency of administration of the therapy recommended for the therapy for use as a single agent is used in accordance with the methods disclosed herein. Recommended doses for approved therapies can be found in the Physician's Desk Reference.

In certain embodiments, a recombinant NDV or an immunogenic composition described herein is administered to a subject concurrently with the administration of one or more additional therapies. In some embodiments, an immunogenic composition comprising recombinant NDV and a pharmaceutical composition comprising one or more additional therapies may be administered concurrently, or before or after each other. In certain embodiments, the immunogenic composition and pharmaceutical composition are administered concurrently to the subject, or within 1 minute, 2 minutes, 5 minutes, 10 minutes, 15 minutes, 20 minutes, 30 minutes, 45 minutes, 60 minutes, 1.5 hours, 2 hours, 3 hours, 4 hours, 5 hours, or 6 hours of each other. In certain embodiments, the first and second pharmaceutical compositions are administered to the subject within 7 days, 2 weeks, 3 weeks, 4 weeks, 5 weeks, 6 weeks, 7 weeks, 8 weeks or 12 weeks of each other. In certain embodiments, the immunogenic composition and pharmaceutical compositions are administered to the subject within 3-6 months, 6-9 months, 6-12 months, or 3 months, 4 months, 6 months, 9 months, or 12 months of each other.

### 5.5.3 Additional Therapies

Additional therapies that can be used in a combination with a recombinant NDV described herein or a composition thereof include, but are not limited to, acetaminophen, ibuprofen, throat lozenges, cough suppressants, inhalers, antibiotics, monoclonal antibodies, and oxygen. In a specific embodiment, the additional therapy is a second recombinant NDV described herein. Additional therapies (e.g., acetaminophen, ibuprofen, throat lozenges, cough suppressants, inhalers, antibiotics, monoclonal antibodies, and oxygen) may also be used in combination with a composition described herein. In a specific embodiment, the additional therapy is a monoclonal antibody, such as sotrovimab. In another specific embodiment, the additional therapy(ies) may include remdesivir, sotrovimab, bamlanivimab plus etesevimab (AIIa), casirivimab plus imdevimab (AIIa), dexamethasone, tocilizumab, oxygen, or a combination thereof.

### 5.5.4 Other Uses of Recombinant NDV

In some embodiments, a recombinant NDV described herein, or an immunogenic composition described herein is administered to a non-human subject (e.g., a mouse, rat, etc.) and the antibodies generated in response to the polypeptide are isolated. In some embodiments, a polynucleotide described herein, a nucleotide sequence described herein, or a vector described herein is administered to a non-human subject (e.g., a mouse, rat, etc.) and the antibodies generated in response to the polypeptide are isolated. In some embodiments, a recombinant protein described herein is administered to a non-human subject (e.g., a mouse, rat, etc.) and the antibodies generated in response to the polypeptide are isolated. Hybridomas may be made and monoclonal antibodies produced as known to one of skill in the art. The antibodies may also be optimized. In some embodiments, the antibodies produced are humanized or chimerized. In certain embodiments, the non-human subject produces human antibodies. The antibodies produced using a recombinant NDV described herein, or immunogenic composition described herein may be optimized, using techniques known to one of skill in the art. In a specific embodiment, antibodies generated using a recombinant NDV described herein, or an immunogenic composition described herein may be used to prevent, treat or prevent and treat COVID-19.

In some embodiments, a recombinant NDV described herein is used in an immunoassay (e.g., an ELISA assay) known to one of skill in the art or described herein to detect antibody specific for SARS-CoV-2 Omicron variant spike protein. In one embodiment, method for detecting the presence of antibody specific to SARS-CoV-2 Omicron variant spike protein, comprising contacting a specimen with the recombinant NDV described herein in an immunoassay (e.g., an ELISA). In some embodiments, a recombinant protein described herein is used in an immunoassay (e.g., an ELISA assay) known to one of skill in the art or described herein to detect antibody specific for SARS-CoV-2 Omicron variant spike protein. In one embodiment, method for detecting the presence of antibody specific to SARS-CoV-2 Omicron variant spike protein, comprising contacting a specimen with a recombinant protein described herein in an immunoassay (e.g., an ELISA). In some embodiments, the specimen is a biological specimen. In a specific embodiment, the biological specimen is blood, plasma or sera from a subject (e.g., a human subject). In other embodiments, the specimen is an antibody or antisera.

### 5.6 BIOLOGICAL ASSAYS

In a specific embodiment, one, two or more of the assays described in Section 6 may be used to characterize a recombinant NDV described herein, a SARS-CoV-2 Omicron variant spike protein or portion thereof (e.g., the ectodomain or receptor binding domain of the SARS-CoV-2 Omicron variant spike protein), a derivative of a SARS-CoV-2 Omicron variant spike protein or portion thereof (e.g., the ectodomain or receptor binding domain of the SARS-CoV-2 Omicron variant spike protein), a recombinant protein described herein, or a chimeric F protein. In another specific embodiment, assays known to one of skill in the art may be used to characterize immunoglobulin samples from a subject (e.g., a human subject) administered a recombinant NDV described herein or a composition described herein. For example, the IgG titer and microneutralization of IgG induced may be assessed as described herein or known to one of skill in the art. In some embodiments, a subject administered a recombinant NDV described herein or a composition described herein is assessed for anti-NDV antibodies as well as anti-SARS-CoV-2 Omicron variant spike protein antibodies. In some embodiments, a subject administered a recombinant NDV described herein or a composition described herein is assessed for anti-SARS-CoV-2 spike protein antibodies that cross-react with the spike protein of SARS-CoV-2 variants other than Omicron.

### 5.6.1 In Vitro Viral Assays

Viral assays include those that indirectly measure viral replication (as determined, e.g., by plaque formation) or the production of viral proteins (as determined, e.g., by western blot analysis) or viral RNAs (as determined, e.g., by RT-PCR or northern blot analysis) in cultured cells in vitro using methods which are well known in the art.

Growth of the recombinant NDVs described herein can be assessed by any method known in the art or described herein (e.g., in cell culture (e.g., cultures of BSTT7 or embryonated chicken cells) (see, e.g., Section 6). Viral titer may be determined by inoculating serial dilutions of a recombinant NDV described herein into cell cultures (e.g., BSTT7 or embryonated chicken cells), chick embryos (e.g., 9 to 11 day old embryonated eggs), or live non-human animals. After incubation of the virus for a specified time, the virus is isolated using standard methods. Physical quantitation of the virus titer can be performed using PCR applied to viral supernatants (Quinn & Trevor, 1997; Morgan et al., 1990), hemagglutination assays, tissue culture infectious doses (TCID50) or egg infectious doses (EID50).

Incorporation of nucleotide sequences encoding a heterologous peptide or protein (e.g., a transgene into the genome of a recombinant NDV described herein can be assessed by any method known in the art or described herein (e.g., in cell culture, an animal model or viral culture in embryonated eggs)). For example, viral particles from cell culture of the allantoic fluid of embryonated eggs can be purified by centrifugation through a sucrose cushion and subsequently analyzed for protein expression by Western blotting using methods well known in the art. In a specific embodiment, a method described in Section 6, infra, is used to assess the incorporation of a transgene into the genome of a recombinant NDV.

Immunofluorescence-based approaches may also be used to detect virus and assess viral growth. Such approaches are well known to those of skill in the art, e.g., fluorescence microscopy and flow cytometry. Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry, 2^{nd} ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, OR; Sigma-Aldrich (2003) Catalogue, St. Louis, MO).

Standard methods of histology of the immune system are described (see, e.g., Muller-Harmelink (ed.) (1986) Human Thymus: Histopathology and Pathology, Springer Verlag, New York, NY; Hiatt, et al. (2000) Color Atlas of Histology, Lippincott, Williams, and Wilkins, Phila, PA; Louis, et al. (2002) Basic Histology: Text and Atlas, McGraw-Hill, New York, NY).

### 5.6.2 Interferon Assays

IFN induction and release induced by a recombinant NDV described herein or a composition described herein may be determined using techniques known to one of skill in the art. For example, the amount of IFN induced in cells following infection with a recombinant NDV described herein may be determined using an immunoassay (e.g., an ELISA or Western blot assay) to measure IFN expression or to measure the expression of a protein whose expression is induced by IFN. Alternatively, the amount of IFN induced may be measured at the RNA level by assays, such as Northern blots and quantitative RT-PCR, known to one of skill in the art. In specific embodiments, the amount of IFN released may be measured using an ELISPOT assay. Further, the induction and release of cytokines and/or interferon-stimulated genes may be determined by, e.g., an immunoassay or ELISPOT assay at the protein level and/or quantitative RT-PCR or northern blots at the RNA level.

### 5.6.3 Toxicity Studies

In some embodiments, the recombinant NDVs described herein or compositions thereof, or combination therapies described herein are tested for cytotoxicity in mammalian, preferably human, cell lines. In certain embodiments, cytotoxicity is assessed in one or more of the following non-limiting examples of cell lines: U937, a human monocyte cell line; primary peripheral blood mononuclear cells (PBMC); Huh7, a human hepatoblastoma cell line; HL60 cells, HT1080, HEK 293T and 293H, MLPC cells, human embryonic kidney cell lines; human melanoma cell lines, such as SkMel2, SkMel-119 and SkMel-197; THP-1, monocytic cells; a HeLa cell line; and neuroblastoma cells lines, such as MC-IXC, SK-N-MC, SK-N-MC, SK-N-DZ, SH-SY5Y, and BE(2)-C. In some embodiments, the ToxLite assay is used to assess cytotoxicity. Many assays well-known in the art can be used to assess viability of cells or cell lines and, thus, determine the cytotoxicity.

Many assays well-known in the art can be used to assess viability of cells or cell lines following infection with a recombinant NDV described herein or a composition thereof, and, thus, determine the cytotoxicity of the recombinant NDV or composition thereof. For example, cell proliferation can be assayed by measuring Bromodeoxyuridine (BrdU) incorporation, (³H) thymidine incorporation, by direct cell count, or by detecting changes in transcription, translation or activity of known genes such as proto-oncogenes (e.g., fos, myc) or cell cycle markers (Rb, cdc2, cyclin A, D1, D2, D3, E, etc.). The levels of such protein and mRNA and activity can be determined by any method well known in the art. For example, protein can be quantitated by known immunodiagnostic methods such as ELISA, Western blotting or immunoprecipitation using antibodies, including commercially available antibodies. mRNA can be quantitated using methods that are well known and routine in the art, for example, using northern analysis, RNase protection, or polymerase chain reaction in connection with reverse transcription. Cell viability can be assessed by using trypan-blue staining or other cell death or viability markers known in the art. In a specific embodiment, the level of cellular ATP is measured to determined cell viability. In preferred embodiments, a recombinant NDV described herein or composition thereof does not kill healthy (i.e., non-cancerous) cells.

In specific embodiments, cell viability may be measured in three-day and seven-day periods using an assay standard in the art, such as the CellTiter-Glo Assay Kit (Promega) which measures levels of intracellular ATP. A reduction in cellular ATP is indicative of a cytotoxic effect. In another specific embodiment, cell viability can be measured in the neutral red uptake assay. In other embodiments, visual observation for morphological changes may include enlargement, granularity, cells with ragged edges, a filmy appearance, rounding, detachment from the surface of the well, or other changes.

The recombinant NDVs described herein or compositions described herein, or combination therapies can be tested for in vivo toxicity in animal models. For example, animals are administered a range of pfu of a recombinant NDV described herein, and subsequently, the animals are monitored over time for various parameters, such as one, two or more of the following: lethality, weight loss or failure to gain weight, and levels of serum markers that may be indicative of tissue damage (e.g., creatine phosphokinase level as an indicator of general tissue damage, level of glutamic oxalic acid transaminase or pyruvic acid transaminase as indicators for possible liver damage). These in vivo assays may also be adapted to test the toxicity of various administration mode and regimen in addition to dosages. See, e.g., the Examples, infra, for assays that may be used to assess toxicity.

The toxicity, efficacy or both of a recombinant NDV described herein or a composition thereof, or a combination therapy described herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapies that exhibit large therapeutic indices are preferred.

The data obtained from the cell culture assays and animal studies can be used in formulating a range of dosage of the therapies for use in subjects.

### 5.6.4 Biological Activity Assays

The recombinant NDVs described herein or compositions described herein, or combination therapies described herein can be tested for biological activity using animal models for inhibiting COVID-19, antibody response to the recombinant NDVs, etc. Such animal model systems include, but are not limited to, rats, mice, hamsters, cotton rats, chicken, cows, monkeys (e.g., African green monkey), pigs, dogs, rabbits, etc.

In a specific embodiment, the recombinant NDVs described herein, compositions described herein, or combination therapies described herein may be tested using animal models for the ability to induce a certain geometric mean titer of antibody (ies) that binds to the SARS-CoV-2 spike protein. An immunoassay, such as an ELISA, or known to one of skill in the art may be used to measure antibody titer. In another specific embodiment, the recombinant NDVs described herein, compositions described herein, or combination therapies described herein may be tested using animal models for the ability to induce antibodies that have neutralizing activity against SARS-CoV-2 spike protein (e.g., SARS-CoV-2 Omicron variant spike protein) in a microneutralization assay. In some embodiments, the recombinant NDVs described herein, compositions described herein, or combination therapies described herein may be tested using animal models for the ability to induce antibodies that neutralize SARS-CoV-2 (e.g., SARS-CoV-2 Omicron virus) in a microneutralization assay. In some embodiments, the recombinant NDVs described herein, compositions described herein, or combination therapies described herein may be tested using animal models for the ability to induce a certain geometric mean titer of antibody (ies) that binds to the SARS-CoV-2 spike protein (e.g., SARS-CoV-2 Omicron variant spike) and neutralizes SARS-CoV-2 (e.g., SARS-CoV-2 Omicron variant) in a microneutralization assay. In some embodiments, the recombinant NDVs described herein or compositions thereof, or combination therapies described herein may be tested using animal models for the ability to induce a certain geometric mean titer of antibody (ies) that binds to the SARS-CoV-2 spike protein (e.g., SARS-CoV-2 Omicron variant spike protein) and neutralizes SARS-CoV-2 (e.g., SARS-CoV-2 Omicron variant) in a microneutralization assay such as described herein. In certain embodiments, the recombinant NDVs described herein, or compositions described herein, or combination therapies described herein may be tested using animal models for the ability to induce a protective immune response. In some embodiments, the recombinant NDVs described herein, or compositions described herein, or combination therapies described herein may be tested using animal models, such as described in Example 5.

In a specific embodiment, a recombinant NDV described herein, a composition described herein, or a combination therapy described herein may be tested in a clinical trial study. In certain embodiments, a recombinant NDV described herein, a composition described herein, or a combination therapy described herein is administered to a human subject. In some embodiments, a human subject administered a recombinant NDV described herein, a composition described herein, or a combination therapy described herein may be assessed for one, two or more, or all of the following may be assessed following administration of a recombinant NDV described herein, or a composition described herein, or a combination therapy described herein: GMT, anti-SARS-CoV-2 spike protein Ig (e.g., IgG, IgA, IgM, etc.), T cell response, NT50 seropositive response, NT80 seropositive response, T cell response, anti-NDV HN antibody, and anti-NDV F antibody.

### 5.6.5 Expression of Transgene

Assays for testing the expression of SARS-CoV-2 Omicron variant spike protein or portion thereof (e.g., SARS-CoV-2 spike protein ectodomain or receptor binding domain), a derivative of a SARS-CoV-2 Omicron variant spike protein or portion thereof (e.g., SARS-CoV-2 Omicron variant spike protein ectodomain or receptor binding domain), or a chimeric F protein in cells infected with a recombinant NDV comprising a packaged genome comprising a transgene that comprises a nucleotide sequence encoding SARS-CoV-2 Omicron variant spike protein or portion thereof (e.g., SARS-CoV-2 Omicron variant spike protein ectodomain or receptor binding domain), a derivative of a SARS-CoV-2 Omicron variant spike protein or portion thereof (e.g., SARS-CoV-2 Omicron variant spike protein ectodomain or receptor binding domain), or chimeric F protein, respectively may be conducted using any assay known in the art, such as, e.g., western blot, immunofluorescence, and ELISA, or any assay described herein. Immunoassays, such as e.g., western blot, immunofluorescence, and ELISA, or another known to one of skill in the art may be used to assess expression of a protein described herein.

In a specific aspect, ELISA is utilized to detect expression of a SARS-CoV-2 Omicron variant spike protein or portion thereof (e.g., SARS-CoV-2 Omicron variant spike protein ectodomain or receptor binding domain), a derivative of a SARS-CoV-2 Omicron variant spike protein or portion thereof (e.g., SARS-CoV-2 Omicron variant spike protein ectodomain or receptor binding domain), or a chimeric F protein in cells infected with a recombinant NDV comprising a packaged genome comprising a transgene that comprises a nucleotide sequence encoding of a SARS-CoV-2 Omicron variant spike protein or portion thereof (e.g., SARS-CoV-2 Omicron variant spike protein ectodomain or receptor binding domain), a derivative of a SARS-CoV-2 Omicron variant spike protein or portion thereof (e.g., SARS-CoV-2 Omicron variant spike protein ectodomain or receptor binding domain), or a chimeric F protein.

In one embodiment, a SARS-CoV-Omicron variant spike protein or portion thereof (e.g., SARS-CoV-2 Omicron variant spike protein ectodomain or receptor binding domain), a derivative of a SARS-CoV-2 Omicron variant spike protein or portion thereof (e.g., SARS-CoV-2 Omicron variant spike protein ectodomain or receptor binding domain), or a chimeric F protein encoded by a packaged genome of a recombinant NDV described herein is assayed for proper folding by testing its ability to bind specifically to an anti-SARS-CoV-2 Omicron variant spike protein using any assay for antibody-antigen interaction known in the art. In another embodiment, a SARS-CoV-2 Omicron variant spike protein or portion thereof (e.g., SARS-CoV-2 Omicron variant spike protein ectodomain or receptor binding domain), a derivative of a SARS-CoV-2 Omicron variant spike protein or portion thereof (e.g., SARS-CoV-2 Omicron variant spike protein ectodomain or receptor binding domain), or a chimeric F protein encoded by a packaged genome of a recombinant NDV described herein is assayed for proper folding by determination of the structure or conformation of the SARS-CoV-2 Omicron variant spike protein or portion thereof (e.g., SARS-CoV-2 Omicron variant spike protein ectodomain or receptor binding domain), a derivative of a SARS-CoV-2 Omicron spike protein or portion thereof (e.g., SARS-CoV-2 Omicron variant spike protein ectodomain or receptor binding domain), or chimeric F protein, respectively using any method known in the art such as, e.g., NMR, X-ray crystallographic methods, or secondary structure prediction methods, e.g., circular dichroism. Additional assays assessing the conformation and antigenicity of SARS-CoV-2 Omicron variant spike protein or portion thereof (e.g., SARS-CoV-2 Omicron variant spike protein ectodomain or receptor binding domain), a derivative of a SARS-CoV-2 Omicron variant spike protein or portion thereof (e.g., SARS-CoV-2 Omicron variant spike protein ectodomain or receptor binding domain), or a chimeric F protein may include, e.g., immunofluorescence microscopy, flow cytometry, western blot, and ELISA may be used. Assays such as, e.g., NMR, X-ray crystallographic methods, secondary structure prediction methods, e.g., circular dichroism, or other assays/techniques known to one of skill in the art may be used to assess the structure or conformation of a protein described herein.

### 5.7 Kits

In one aspect, provided herein is a pharmaceutical pack or kit comprising one or more containers filled with one or more of the ingredients of a composition (e.g., an immunogenic compositions) described herein. In a specific embodiment, provided herein is a pharmaceutical pack or kit comprising a container, wherein the container comprises a recombinant NDV described herein. In a specific embodiment, provided herein is a pharmaceutical pack or kit comprising a container, wherein the container comprises an immunogenic composition described herein. The immunogenic composition may be monovalent or multivalent. Optionally associated with such container(s) can be a notice in the form prescribed by a governmental agency regulating the manufacture, use or sale of pharmaceuticals or biological products, which notice reflects approval by the agency of manufacture, use or sale for human administration.

In another embodiment, provided herein is a kit comprising in one or more containers filled with one or more recombinant NDVs described herein. In another embodiment, provided herein is a kit comprising in one or more containers one or more transgenes described herein. In another embodiment, provided herein is a kit comprising in one or more containers one or more nucleotide sequences comprising the genome of NDV and a transgene described herein. In another embodiment, provided herein is a kit comprising, in a container, a vector comprising a transgene described herein.

In another embodiment, provided herein is a kit comprising in one or more containers filled with one or more recombinant proteins described herein or nucleic acid sequence described herein. In another embodiment, provided herein is a kit comprising in one or more containers filled with one or more polynucleotides described herein or nucleic acid sequence described herein. In another embodiment, provided herein is a kit comprising, in a container, a vector comprising a polynucleotide described herein, a nucleotide sequence described herein, or a nucleic acid sequence described herein.

In a specific embodiment, provided herein is a kit comprising, in a container, a nucleotide sequence comprising a transgene described herein and (1) a NDV F transcription unit, (2) a NDV NP transcription unit, (3) a NDV M transcription unit, (4) a NDV L transcription unit, (5) a NDV P transcription unit, and (6) a NDV HN transcription unit. In some embodiments, the NDV F transcription unit encodes a NDV F protein comprising a leucine to alanine amino acid substitution at the amino residue corresponding to amino acid residue 289 of the LaSota NDV strain.

In a specific embodiment, provided herein is a kit comprising, in a container, a vector comprising a nucleotide sequence, wherein the nucleotide sequence comprises a transgene described herein and (1) a NDV F transcription unit, (2) a NDV NP transcription unit, (3) a NDV M transcription unit, (4) a NDV L transcription unit, (5) a NDV P transcription unit, and (6) a NDV HN transcription unit. In some embodiments, the NDV F transcription unit encodes a NDV F protein comprising a leucine to alanine amino acid substitution at the amino residue corresponding to amino acid residue 289 of the LaSota NDV strain.

### 5.8 Sequences

TABLE 1
cDNA of genome of NDV Strains

| | | SEQ ID |
|---|---|---|
| Description | Sequence | NO: |
| cDNA of | accaaacagagaatccgtgagttacgataaaaggcgaaggagcaattgaagtcgcacggg | SEQ ID |
| genomic | tagaaggtgtgaatctcgagtgcgagcccgaagcacaaactcgagaaagccttctgccaac | NO: 1 |
| sequence of | atgtcttccgtatttgatgagtacgaacagctcctcgcggctcagactcgccccaatggagct | |
| NDV strain | catggagggggagaaaaagggagtaccttaaaagtagacgtcccggtattcactcttaaca | |
| LaSota | gtgatgacccagaagatagatggagctttgtggtattctgcctccggattgctgttagcgaag | |
| | atgccaacaaaccactcaggcaaggtgctctcatatctcttttatgctcccactcacaggtaat | |
| | gaggaaccatgttgccCttgcagggaaacagaatgaagccacattggccgtgcttgagatt | |
| | gatggctttgccaacggcacgccccagttcaacaataggagtggagtgtctgaagagagag | |
| | cacagagatttgcgatgatagcaggatctctccctcgggcatgcagcaacggaaccccgttc | |
| | gtcacagccggggcCgaagatgatgcaccagaagacatcaccgataccctggagaggat | |
| | cctctctatccaggctcaagtatgggtcacagtagcaaaagccatgactgcgtatgagactg | |
| | cagatgagtcggaaacaaggcgaatcaataagtatatgcagcaaggcagggtccaaaaga | |
| | aatacatcctctaccccgtatgcaggagcacaatccaactcacgatcagacagtctcttgcag | |
| | tccgcatctttttggttagcgagctcaagagaggccgcaacacggcaggtggtacctctactt | |
| | attataacctggtaggggacgtagactcatacatcaggaataccgggcttactgcattcttctt | |
| | gacactcaagtacggaatcaacaccaagacatcagcccttgcacttagtagcctctcaggcg | |
| | acatccagaagatgaagcagctcatgcgtttgtatcggatgaaaggagataatgcgccgtac | |
| | atgacattacttggtgatagtgaccagatgagctttgcgcctgccgagtatgcacaactttact | |
| | cctttgccatgggtatggcatcagtcctagataaaggtactgggaaataccaatttgccaggg | |
| | actttatgagcacatcattctggagacttggagtagagtacgctcaggctcagggaagtagc | |
| | attaacgaggatatggctgccgagctaaagctaaccccagcagcaaGgaGgggcctggc | |
| | agctgctgcccaacgggtctccgaGgaGaccagcagcataGacatgcctactcaacaag | |
| | tcggagtcctcactgggcttagcgagggggggtcccaagctctacaaggcggatcgaata | |
| | gatcgcaagggcaaccagaagccggggatggggagacccaattcctggatctgatgaga | |
| | gcggtagcaaatagcatgagggaggcgccaaactctgcacagggcactccccaatcggg | |
| | gcctcccccaactcctgggccatoccaagataacgacaccgactgggggtattgatggaca | |
| | aaacccagcctgcttccacaaaaacatcccaatgccctcacccgtagtcgacccctcgatttg | |
| | cggctctatatgaccacaccctcaaacaaacatccccctotttcctccctccccctgctgtaca | |
| | actAcgTacgccctagataccacaggcacaatgcggctcactaacaatcaaaacagagc | |
| | cgagggaattagaaaaaagtacgggtagaagagggatattcagagatcagggcaagtctc | |
| | ccgagtctctgctctctcctctacctgatagaccaggacaaacatggccacctttacagatgc | |
| | agagatcgacgagctatttgagacaagtggaactgtcattgacaacataattacagcccagg | |
| | gtaaaccagcagagactgttggaaggagtgcaatcccacaaggcaagaccaaggtgctga | |
| | gcgcagcatgggagaagcatgggagcatccagccaccggccagtcaagacaaccccgat | |
| | cgacaggacagatctgacaaacaaccatccacacccgagcaaacgaccccgcatgacag | |
| | cccgccggccacatccgccgaccagccccccacccaggccacagacgaagccgtcgac | |
| | acacagCtcaggaccggagcaagcaactctctgctgttgatgcttgacaagctcagcaata | |
| | aatcgtccaatgctaaaaagggcccatggtcgagcccccaagaggggaatcaccaacgtc | |
| | cgactcaacagcaggggagtcaacccagtcgcggaaacagtcaggaaagaccgcagaa | |
| | ccaagtcaaggccgcccctggaaaccagggcacagacgtgaacacagcatatcatggac | |
| | aatgggaggagtcacaactatcagctggtgcaacccctcatgctctccgatcaaggcagag | |
| | ccaagacaatacccttgtatctgcggatcatgtccagccacctgtagactttgtgcaagcgat | |
| | gatgtctatgatggaggcgatatcacagagagtaagtaaggttgactatcagctagatcttgtc | |
| | ttgaaacagacatcctccatccctatgatgoggtccgaaatccaacagctgaaaacatctgtt | |
| | gcagtcatggaagccaacttgggaatgatgaagattctggatcccggttgtgccaacatttca | |
| | tctctgagtgatctacgggcagttgcccgatctcacccggttttagtttcaggccctggagacc | |
| | cctctccctatgtgacacaaggaggcgaaatggcacttaataaactttcgcaaccagtgcca | |
| | catccatctgaattgattaaacccgccactgcatgcgggcctgatataggagtggaaaagga | |
| | cactgtccgtgcattgatcatgtcacgcccaatgcacccgagttcttcagccaagctcctaag | |
| | caagttagatgcagccgggtcgatcgaggaaatcaggaaaatcaagcgccttgctctaaat | |
| | ggctaattactactgccacacgtagcgggtccctgtccactcggcatcacacggaatctgca | |
| | ccgagttcccccccgcGgacccaaggtccaactctccaagcggcaatcctctctcgcttcct | |
| | cagccccactgaatgAtcgcgtaaccgtaattaatctagctacatttaagattaagaaaaaat | |
| | acgggtagaattggagtgccccaattgtgccaagatggactcatctaggacaattgggctgt | |
| | actttgattctgcccattcttctagcaacctgttagcatttccgatcgtcctacaagAcacagga | |
| | gatgggaagaagcaaatcgccccgcaatataggatccagcgccttgacttgtggactgata | |
| | gtaaggaggactcagtattcatcaccacctatggattcatctttcaagttgggaatgaagaagc | |
| | cacCgtcggcatgatcgatgataaacccaagcgcgagttactttccgctgcgatgctctgcc | |
| | taggaagcgtcccaaataccggagaccttattgagctggcaagggcctgtctcactatgata | |
| | gtcacatgcaagaagagtgcaactaatactgagagaatggttttctcagtagtgcaggcacc | |
| | ccaagtgctgcaaagctgtagggttgtggcaaacaaatactcatcagtgaatgcagtcaagc | |
| | acgtgaaagcgccagagaagattcccgggagtggaaccctagaatacaaggtgaactttgt | |
| | ctccttgactgtggtaccgaagaGggatgtctacaagatcccagctgcagtattgaaggtttc | |
| | tggctcgagtctgtacaatcttgcgctcaatgtcactattaatgtggaggtagacccgaggagt | |
| | cctttggttaaatctCtgtctaagtctgacagcggatactatgctaacctcttcttgcatattgga | |
| | cttatgaccacTgtagataggaaggggaagaaagtgacatttgacaagctggaaaagaaa | |
| | ataaggagccttgatctatctgtcgggctcagtgatgtgctcgggccttccgtgttggtaaaag | |
| | caagaggtgcacggactaagcttttggcacctttcttctctagcagtgggacagcctgctatc | |
| | ccatagcaaatgcttctcctcaggtggccaagatactctggagtcaaaccgcgtgcctgcgg | |
| | agcgttaaaatcattatccaagcaggtacccaacgcgctgtcgcagtgaccgccgaccacg | |
| | aggttacctctactaagctggagaaggggcacacccttgccaaatacaatccttttaagaaat | |
| | aagctgcgtctctgagattgcgctccgcccactcacccagatcatcatgacacaaaaaacta | |
| | atctgtcttgattatttacagttagtttacctgtctatcaagttagaaaaaacacgggtagaagatt | |
| | ctggatcccggttggcgccctccaggtgcaagatgggctccagaccttctaccaagaaccc | |
| | agcacctatgatgctgactatccgggttgcgctggtactgagttgcatctgtccggcaaactc | |
| | cattgatggcaggcctcttgcagctgcaggaattgtggttacaggagacaaagccgtcaaca | |
| | tatacacctcatcccagacaggatcaatcatagttaagctcctcccgaatctgcccaaggata | |
| | aggaggcatgtgcgaaagcccccttggatgcatacaacaggacattgaccactttgctcacc | |
| | ccccttggtgactctatccgtaggatacaagagtctgtgactacatctggaggggggagaca | |
| | ggggcgccttataggcgccattattggcggtgtggctcttggggttgcaactgccgcacaaa | |
| | taacagcggccgcagctctgatacaagccaaacaaaatgctgccaacatcctccgacttaaa | |
| | gagagcattgccgcaaccaatgaggctgtgcatgaggtcactgacggattatcgcaactag | |
| | cagtggcagttgggaagatgcagcagtttgttaatgaccaatttaataaaacagctcaggaat | |
| | tagactgcatcaaaattgcacagcaagttggtgtagagctcaacctgtacctaaccgaattga | |
| | ctacagtattcggaccacaaatcacttcacctgctttaaacaagctgactattcaggcactttac | |
| | aatctagctggtggaaatatggattacttattgactaagttaggtgtagggaacaatcaactca | |
| | gctcattaatcggtagcggcttaatcaccggtaaccctattctatacgactcacagactcaact | |
| | cttgggtatacaggtaactctaccttcagtcgggaacctaaataatatgcgtgccacctacttg | |
| | gaaaccttatccgtaagcacaaccaggggatttgcctcggcacttgtcccAaaagtggtga | |
| | cacaggtcggttctgtgatagaagaacttgacacctcatactgtatagaaactgacttagattta | |
| | tattgtacaagaatagtaacgttccctatgtcccctggtatttattcctgcttgagcggcaatacg | |
| | tcggcctgtatgtactcaaagaccgaaggcgcacttactacaccatacatgactatcaaaggt | |
| | tcagtcatcgccaactgcaagatgacaacatgtagatgtgtaaaccccccgggtatcatatcg | |
| | caaaactatggagaagccgtgtctctaatagataaacaatcatgcaatgttttatccttaggcg | |
| | ggataactttaaggctcagtggggaattcgatgtaacttatcagaagaatatctcaatacaaga | |
| | ttctcaagtaataataacaggcaatcttgatatctcaactgagcttgggaatgtcaacaactcg | |
| | atcagtaatgctttgaataagttagaggaaagcaacagaaaactagacaaagtcaatgtcaa | |
| | actgactagcacatctgctctcattacctatatcgttttgactatcatatctcttgtttttggtata | |
| | cttagcctgattctagcatgctacctaatgtacaagcaaaaggcgcaacaaaagaccttattatgg | |
| | cttgggaataatactctagatcagatgagagccactacaaaaatgtgaacacagatgaggaa | |
| | cgaaggtttccctaatagtaatttgtgtgaaagttctggtagtctgtcagttcagagagttaaga | |
| | aaaaactaccggttgtagatgaccaaaggacgatatacgggtagaacggtaagagaggcc | |
| | gcccctcaattgcgagccaggcttcacaacctccgttctaccgcttcaccgacaacagtcctc | |
| | aatcatggaccgcgccgttagccaagttgcgttagagaatgatgaaagagaggcaaaaaat | |
| | acatggcgcttgatattccggattgcaatcttattcttaacagtagtgaccttggctatatctgta | |
| | gcctcccttttatatagcatgggggctagcacacctagcgatcttgtaggcataccgactagg | |
| | atttccagggcagaagaaaagattacatctacacttggttccaatcaagatgtagtagatagg | |
| | atatataagcaagtggcccttgagtctccgttggcattgttaaatactgagaccacaattatgaa | |
| | cgcaataacatctctctcttatcagattaatggagctgcaaacaacagtgggggggggcac | |
| | ctatccatgacccagattatataggggggataggcaaagaactcattgtagatgatgctagtg | |
| | atgtcacatcattctatccctctgcatttcaagaacatctgaattttatcccggcgcctactacag | |
| | gatcaggttgcactcgaataccctcatttgacatgagtgctacccattactgctacacccataat | |
| | gtaatattgtctggatgcagagatcactcacattcatatcagtatttagcacttggtgtgctccg | |
| | gacatctgcaacagggagggtattcttttctactctgcgttccatcaacctggacgacaccca | |
| | aaatcggaagtcttgcagtgtgagtgcaactcccctgggttgtgatatgctgtgctcgaaagt | |
| | cacggagacagaggaagaagattataactcagctgtccctacgcggatggtacatgggag | |
| | gttagggttcgacggccagtaccacgaaaaggacctagatgtcacaacattattcggggact | |
| | gggtggccaactacccaggagtagggggtggatcttttattgacagccgcgtatggttctca | |
| | gtctacggagggttaaaacccaattcacccagtgacactgtacaggaagggaaatatgtgat | |
| | atacaagcgatacaatgacacatgcccagatgagcaagactaccagattcgaatggccaag | |
| | tcttcgtataagcctggacggtttggtgggaaacgcatacagcaggctatcttatctatcaagg | |
| | tgtcaacatccttaggcgaagacccggtactgactgtaccgcccaacacagtcacactcatg | |
| | ggggccgaaggcagaattctcacagtagggacatctcatttcttgtatcaacgagggtcatca | |
| | tacttctctcccgcgttattatatcctatgacagtcagcaacaaaacagccactcttcatagtcct | |
| | tatacattcaatgccttcactcggccaggtagtatcccttgccaggcttcagcaagatgcccca | |
| | actcgtgtgttactggagtctatacagatccatatcccctaatcttctatagaaaccacaccttg | |
| | cgaggggtattcgggacaatgcttgatggtgtacaagcaagacttaaccctgcgtctgcagt | |
| | attcgatagcacatcccgcagtcgcattactcgagtgagttcaagcagtaccaaagcagcat | |
| | acacaacatcaacttgttttaaagtggtcaagactaataagacctattgtctcagcattgctgaa | |
| | atatctaatactctcttcggagaattcagaatcgtcccgttactagttgagatcctcaaagatga | |
| | cggggttagagaagccaggtctggctagttgagtcaattataaaggagttggaaagatggca | |
| | ttgtatcacctatcttctgcgacatcaagaatcaaaccgaatgccggcgcgtgctcgaattcca | |
| | tgttgccagttgaccacaatcagccagtgctcatgcgatcagattaagccttgtcaAtaGtct | |
| | cttgattaagaaaaaatgtaagtggcaatgagatacaaggcaaaacagctcatggtTaaCa | |
| | atacgggtaggacatggcgagctccggtcctgaaagggcagagcatcagattatcctacca | |
| | gagTcacacctgtcttcaccattggtcaagcacaaactactctattactggaaattaactggg | |
| | ctaccgcttcctgatgaatgtgacttcgaccacctcattctcagccgacaatggaaaaaaata | |
| | cttgaatcggcctctcctgatactgagagaatgataaaactcggaagggcagtacaccaaac | |
| | tcttaaccacaattccagaataaccggagtgctccaccccaggtgtttagaaGaactggcta | |
| | atattgaggtcccagattcaaccaacaaatttcggaagattgagaagaagatccaaattcaca | |
| | acacgagatatggagaactgttcacaaggctgtgtacgcatatagagaagaaactgctggg | |
| | gtcatcttggtctaacaatgtcccccggtcagaggagttcagcagcattcgtacggatccggc | |
| | attctggtttcactcaaaatggtccacagccaagtttgcatggctccatataaaacagatccag | |
| | aggcatctgatggtggcagctaGgacaaggtctgcggccaacaaattggtgatgctaaccc | |
| | ataaggtaggccaagtctttgtcactcctgaacttgtcgttgtgacgcatacgaatgagaacaa | |
| | gttcacatgtcttacccaggaacttgtattgatgtatgcagatatgatggagggcagagatatg | |
| | gtcaacataatatcaaccacggcggtgcatctcagaagcttatcagagaaaattgatgacattt | |
| | tgcggttaatagacgctctggcaaaagacttgggtaatcaagtctacgatgttgtatcactaat | |
| | ggagggatttgcatacggagctgtccagctactcgagccgtcaggtacatttgcaggagattt | |
| | cttcgcattcaacctgcaggagcttaaagacattctaattggcctcctccccaatgatatagca | |
| | gaatccgtgactcatgcaatcgctactgtattctctggtttagaacagaatcaagcagctgaga | |
| | tgttgtgtctgttgcgtctgtggggtcacccactgcttgagtcccgtattgcagcaaaggcagt | |
| | caggagccaaatgtgcgcaccgaaaatggtagactttgatatgatccttcaggtactgtctttc | |
| | ttcaagggaacaatcatcaacgggtacagaaagaagaatgcaggtgtgtggccgcgagtc | |
| | aaagtggatacaatatatgggaaggtcattgggcaactacatgcagattcagcagagatttca | |
| | cacgatatcatgttgagagagtataagagtttatctgcacttgaatttgagccatgtatagaatat | |
| | gaccctgtcaccaacctgagcatgttcctaaaagacaaggcaatcgcacaccccaacgata | |
| | attggcttgcctcgtttaggcggaaccttctctccgaagaccagaagaaacatgtaaaagaa | |
| | gcaacttcgactaatcgcctcttgatagagtttttagagtcaaatgattttgatccatataaagag | |
| | atggaatatctgacgacccttgagtaccttagagatgacaatgtggcagtatcatactcgctca | |
| | aggagaaggaagtgaaagttaatggacggatcttcgctaagctgacaaagaagttaaggaa | |
| | ctgtcaggtgatggcggaagggatcctagccgatcagattgcacctttctttcagggaaatgg | |
| | agtcattcaggatagcatatccttgaccaagagtatgctagcgatgagtcaactgtcttttaaca | |
| | gcaataagaaacgtatcactgactgtaaagaaagagtatcttcaaaccgcaatcatgatccga | |
| | aaagcaagaaccgtcggagagttgcaaccttcataacaactgacctgcaaaagtactgtctt | |
| | aattggagatatcagacaatcaaattgttcgctcatgccatcaatcagttgatgggcctacctc | |
| | acttcttcgaatggattcacctaagactgatggacactacgatgttcgtaggagaccctttcaat | |
| | cctccaagtgaccctactgactgtgacctctcaagagtccctaatgatgacatatatattgtca | |
| | gtgccagagggggtatcgaaggattatgccagaagctatggacaatgatctcaattgctgca | |
| | atccaacttgctgcagctagatcgcattgtcgtgttgcctgtatggtacagggtgataatcaag | |
| | taatagcagtaacgagagaggtaagatcagacgactctccggagatggtgttgacacagttg | |
| | catcaagccagtgataatttcttcaaggaattaattcatgtcaatcatttgattggccataatttga | |
| | aggatcgtgaaaccatcaggtcagacacattcttcatatacagcaaacgaatcttcaaagatg | |
| | gagcaatcctcagtcaagtcctcaaaaattcatctaaattagtgctagtgtcaggtgatctcagt | |
| | gaaaacaccgtaatgtcctgtgccaacattgcctctactgtagcacggctatgcgagaacgg | |
| | gcttcccaaagacttctgttactatttaaactatataatgagttgtgtgcagacatactttgactct | |
| | gagttctccatcaccaacaattcgcaccccgatcttaatcagtcgtggattgaggacatctcttt | |
| | tgtgcactcatatgttctgactcctgcccaattagggggactgagtaaccttcaatactcaagg | |
| | ctctacactagaaatatcggtgacccggggactactgcttttgcagagatcaagcgactaga | |
| | agcagtgggattactgagtcctaacattatgactaatatcttaactaggccgcctgggaatgg | |
| | agattgggccagtctgtgcaacgacccatactctttcaattttgagactgttgcaagcccaaat | |
| | attgttcttaagaaacatacgcaaagagtcctatttgaaacttgttcaaatcccttattgtctgga | |
| | gtgcacacagaggataatgaggcagaagagaaggcattggctgaattcttgcttaatcaaga | |
| | ggtgattcatccccgcgttgcgcatgccatcatggaggcaagctctgtaggtaggagaaag | |
| | caaattcaagggcttgttgacacaacaaacaccgtaattaagattgcgcttactaggaggcca | |
| | ttaggcatcaagaggctgatgcggatagtcaattattctagcatgcatgcaatgctgtttagag | |
| | acgatgttttttcctccagtagatccaaccaccccttagtctcttctaatatgtgttctctgacact | |
| | ggcagactatgcacggaatagaagctggtcacctttgacgggaggcaggaaaatactgggt | |
| | gtatctaatcctgatacgatagaactcgtagagggtgagattcttagtgtaagcggagggtgt | |
| | acaagatgtgacagcggagatgaacaatttacttggttccatcttccaagcaatatagaattga | |
| | ccgatgacaccagcaagaatcctccgatgagggtaccatatctcgggtcaaagacacagga | |
| | gaggagagctgcctcacttgcaaaaatagctcatatgtcgccacatgtaaaggctgccctaa | |
| | gggcatcatccgtgttgatctgggcttatggggataatgaagtaaattggactgctgctcttac | |
| | gattgcaaaatctcggtgtaatgtaaacttagagtatcttcggttactgtcccctttacccacgg | |
| | ctgggaatcttcaacatagactagatgatggtataactcagatgacattcacccctgcatctct | |
| | ctacaggGtgtcaccttacattcacatatccaatgattctcaaaggctgttcactgaagaagg | |
| | agtcaaagaggggaatgtggtttaccaacagatcatgctcttgggtttatctctaatcgaatcg | |
| | atctttccaatgacaacaaccaggacatatgatgagatcacactgcacctacatagtaaattta | |
| | gttgctgtatcagagaagcacctgttgcggttcctttcgagctacttggggtggtaccggaact | |
| | gaggacagtgacctcaaataagtttatgtatgatcctagccctgtatcggagggagactttgc | |
| | gagacttgacttagctatcttcaagagttatgagcttaatctggagtcatatcccacgatagag | |
| | ctaatgaacattctttcaatatccagcgggaagttgattggccagtctgtggtttcttatgatgaa | |
| | gatacctccataaagaatgacgccataatagtgtatgacaatacccgaaattggatcagtgaa | |
| | gctcagaattcagatgtggtccgcctatttgaatatgcagcacttgaagtgctcctcgactgttc | |
| | ttaccaactctattacctgagagtaagaggcctGgacaatattgtcttatatatgggtgatttata | |
| | caagaatatgccaggaattctactttccaacattgcagctacaatatctcatcccgtcattcattc | |
| | aaggttacatgcagtgggcctggtcaaccatgacggatcacaccaacttgcagatacggatt | |
| | ttatcgaaatgtctgcaaaactattagtatcttgcacccgacgtgtgatctccggcttatattcag | |
| | gaaataagtatgatctgctgttcccatctgtcttagatgataacctgaatgagaagatgcttcag | |
| | ctgatatcccggttatgctgtctgtacacggtactctttgctacaacaagagaaatcccgaaaa | |
| | taagaggcttaactgcagaagagaaatgttcaatactcactgagtatttactgtcggatgctgt | |
| | gaaaccattacttagccccgatcaagtgagctctatcatgtctcctaacataattacattcccag | |
| | ctaatctgtactacatgtctcggaagagcctcaatttgatcagggaaagggaggacagggat | |
| | actatcctggcgttgttgttcccccaagagccattattagagttcccttctgtgcaagatattggt | |
| | gctcgagtgaaagatccattcacccgacaacctgcggcatttttgcaagagttagatttgagt | |
| | gctccagcaaggtatgacgcattcacacttagtcagattcatcctgaactcacatctccaaatc | |
| | cggaggaagactacttagtacgatacttgttcagagggatagggactgcatcttcctcttggta | |
| | taaggcatctcatctcctttctgtacccgaggtaagatgtgcaagacacgggaactccttatac | |
| | ttagctgaagggagcggagccatcatgagtcttctcgaactgcatgtaccacatgaaactatc | |
| | tattacaatacgctcttttcaaatgagatgaaccccccgcaacgacatttcgggcegacccca | |
| | actcagtttttgaattcggttgtttataggaatctacaggcggaggtaacatgcaaagatggatt | |
| | tgtccaagagttccgtccattatggagagaaaatacagaggaaagCgacctgacctcagat | |
| | aaagTagtggggtatattacatctgcagtgccctacagatctgtatcattgctgcattgtgaca | |
| | ttgaaattcctccagggtccaatcaaagcttactagatcaactagctatcaatttatctctgattg | |
| | ccatgcattctgtaagggagggcggggtagtaatcatcaaagtgttgtatgcaatgggatact | |
| | actttcatctactcatgaacttgtttgctccgtgttccacaaaaggatatattctctctaatggttat | |
| | gcatgtcgaggagatatggagtgttacctggtatttgtcatgggttacctgggcgggcctaca | |
| | tttgtacatgaggtggtgaggatggcGaaaactctggtgcagcggcacggtacgctTttgt | |
| | ctaaatcagatgagatcacactgaccaggttattcacctcacagcggcagcgtgtgacagac | |
| | atcctatccagtcctttaccaagattaataaagtacttgaggaagaatattgacactgcgctgat | |
| | tgaagccgggggacagcccgtccgtccattctgtgcggagagtctggtgagcacgctagc | |
| | gaacataactcagataacccagatCatcgctagtcacattgacacagttatccggtctgtgat | |
| | atatatggaagctgagggtgatctcgctgacacagtatttctatttaccccttacaatctctctac | |
| | tgacgggaaaaagaggacatcacttaAacagtgcacgagacagatcctagaggttacaat | |
| | actaggtcttagagtcgaaaatctcaataaaataggcgatataatcagcctagtgcttaaagg | |
| | catgatctccatggaggaccttatcccactaaggacatacttgaagcatagtacctgccctaa | |
| | atatttgaaggctgtcctaggtattaccaaactcaaagaaatgtttacagacacttctgtaCtgt | |
| | acttgactcgtgctcaacaaaaattctacatgaaaactataggcaatgcagtcaaaggatatta | |
| | cagtaactgtgactcttaacgaaaatcacatattaataggctccttttttggccaattgtattcttgt | |
| | tgatttaatcatattatgttagaaaaaagttgaaccctgactccttaggactcgaattcgaactca | |
| | aataaatgtcttaaaaaaaggttgcgcacaattattcttgagtgtagtctcgtcattcaccaaatc | |
| | tttgtttggt | |
| cDNA of | ACCAAACAGAGAATCCGTAAGTTACGATAAAAGGCGA | SEQ ID |
| genomic | AGGAGCAATTGAAGTCGCACGGGTAGAAGGTGTGAATC | NO: 2 |
| sequence of | TCGAGTGCGAGCCCGAAGCACAAACTCGAGGAAGCCTT | |
| NDV strain | CTGCCAACATGTCTTCCGTATTCGACGAGTACGAACAG | |
| Hitchner B1 | CTCCTCGCGGCTCAGACTCGCCCCAATGGAGCTCATGG | |
| | AGGGGGGGAGAAAGGGAGTACCTTAAAAGTAGACGTC | |
| | CCGGTATTCACTCTTAACAGTGATGACCCAGAAGATAG | |
| | GTGGAGCTTTGTGGTATTCTGCCTCCGGATTGCTGTTAG | |
| | CGAAGATGCCAACAAACCACTCAGGCAAGGTGCTCTCA | |
| | TATCTCTTTTATGCTCCCACTCACAGGTAATGAGGAACC | |
| | ATGTTGCCCTTGCAGGGAAACAGAATGAAGCCACATTG | |
| | GCCGTGCTTGAGATTGATGGCTTTGCCAACGGCACGCC | |
| | CCAGTTCAACAATAGGAGTGGAGTGTCTGAAGAGAGAG | |
| | CACAGAGATTTGCGATGATAGCAGGATCTCTCCCTCGG | |
| | GCATGCAGCAACGGCACCCCGTTCGTCACAGCCGGGGC | |
| | TGAAGATGATGCACCAGAAGACATCACCGATACCCTGG | |
| | AGAGGATCCTCTCTATCCAGGCTCAAGTATGGGTCACA | |
| | GTAGCAAAAGCCATGACTGCGTATGAGACTGCAGATGA | |
| | GTCGGAAACAAGGCGAATCAATAAGTATATGCAGCAAG | |
| | GCAGGGTCCAAAAGAAATACATCCTCTACCCCGTATGC | |
| | AGGAGCACAATCCAACTCACGATCAGACAGTCTCTTGC | |
| | AGTCCGCATCTTTTTGGTTAGCGAGCTCAAGAGAGGCC | |
| | GCAACACGGCAGGTGGTACCTCTACTTATTATAACCTA | |
| | GTAGGGGACGTAGACTCATATATCAGGAATACCGGGCT | |
| | TACTGCATTCTTCTTGACACTCAAGTACGGAATCAACAC | |
| | CAAGACATCAGCCCTTGCACTTAGTAGCCTCTCAGGCG | |
| | ACATCCAGAAGATGAAGCAGCTCATGCGTTTGTATCGG | |
| | ATGAAAGGAGATAATGCGCCGTACATGACATTACTTGG | |
| | TGATAGTGACCAGATGAGCTTTGCGCCTGCCGAGTATG | |
| | CACAACTTTACTCCTTTGCCATGGGTATGGCATCAGTCC | |
| | TAGATAAAGGTACTGGGAAATACCAATTTGCCAGGGAC | |
| | TTTATGAGCACATCATTCTGGAGACTTGGAGTAGAGTA | |
| | CGCTCAGGCTCAGGGAAGTAGCATTAACGAGGATATGG | |
| | CTGCCGAGCTAAAGCTAACCCCGGCAGCAAGGAGGGGC | |
| | CTGGCAGCTGCTGCCCAACGAGTCTCCGAGGTGACCAG | |
| | CAGCATAGACATGCCTACTCAACAAGTCGGAGTCCTCA | |
| | CTGGGCTTAGCGAGGGGGGATCCCAAGCCCTACAAGGC | |
| | GGATCGAATAGATCGCAAGGGCAACCAGAAGCCGGGG | |
| | ATGGGGAGACCCAATTCCTGGATCTGATGAGAGCGGTA | |
| | GCAAATAGCATGAGGGAGGCGCCAAACTCTGCACAGG | |
| | GCACTCCCCAATCGGGGCCTCCCCCAACTCCTGGGCCAT | |
| | CCCAAGATAACGACACCGACTGGGGGTATTGATTGACA | |
| | AAACCCAGCCTGCTTCTACAAGAACATCCCAATGCTCTC | |
| | ACCCGTAGTCGACCCCTCGATTTGCGGCTCTATATGACC | |
| | ACACCCTCAAACAAACATCCCCCTCTTTCCTCCCTCCCC | |
| | CTGCTGTACAACTCCGCACGCCCTAGATACCACAGGCA | |
| | CACCGCGGCTCACTAACAATCAAAACAGAGCCGAGGGA | |
| | ATTAGAAAAAAGTACGGGTAGAAGAGGGATATTCAGA | |
| | GATCAGGGCAAGTCTCCCGAGTCTCTGCTCTCTCCTCTA | |
| | CCTGATAGACCAGGACAAACATGGCCACCTTTACAGAT | |
| | GCAGAGATCGACGAGCTATTTGAGACAAGTGGAACTGT | |
| | CATTGACAACATAATTACAGCCCAGGGTAAACCAGCAG | |
| | AGACTGTTGGAAGGAGTGCAATCCCACAGGGCAAGACC | |
| | AAGGTGCTGAGCGCAGCATGGGAGAAGCATGGGAGCA | |
| | TCCAGCCACCGGCCAGTCAAGACAACCTCGATCGACAG | |
| | GACAGATCTGACAAACAACCATCCACACCCGAGCAAAC | |
| | GACCCCGCACGACAGCCCGCCGGCCACATCCGCTGACC | |
| | AGCCCCCCACCCAGGCCACAGACGAAGCCGTCGACACA | |
| | CAGCTCAGGACCGGAGCAAGCAACTCTCTGCTGTTGAT | |
| | GCTTGACAAGCTCAGCAATAAATCGTCCAATGCTAAAA | |
| | AGGGCCCATGGTCGAGCCCCCAAGAGGGGAATCACCAA | |
| | CGTCCGACTCAACAGCAGGGGAGTCAACCCAGTCGCGG | |
| | AAACAGCCAGGAAAGACTGCAGAACCAAGTCAAGGCC | |
| | GCCCCTGGAAACCAGGGCACAGACGTGAACACAGCATA | |
| | TCATGGACAATGGGAGGAGTCACAACTATCAGCTGGTG | |
| | CAACCCCTCATGCTCTCCGATCAAGGCAGAGCCAAGAC | |
| | AATACCCTTGTATCTGCGGATCATGTCCAGCCACCTGTA | |
| | GACTTTGTGCAAGCGATGATGTCTATGATGGGGGCGAT | |
| | ATCACAGAGAGTAAGTAAGGTTGACTATCAGCTAGATC | |
| | TTGTCTTGAAACAGACATCCTCCATCCCTATGATGCGGT | |
| | CCGAAATCCAACAGCTGAAAACATCTGTTGCAGTCATG | |
| | GAAGCCAACTTGGGAATGATGAAGATTCTGGATCCCGG | |
| | TTGTGCCAACATTTCATCTCTGAGTGATCTACGGGCAGT | |
| | TGCCCGATCTCACCCGGTTTTAGTTTCAGGCCCTGGAGA | |
| | CCCATCTCCCTATGTGATACAAGGAGGCGAAATGGCAC | |
| | TTAATAAACTTTCGCAACCAGTGCCACATCCATCTGAAT | |
| | TGATTAAACCCGCCACTGCATGCGGGCCTGATATAGGA | |
| | GTGGAGAGGGACACTGTCCGTGCATTGATCATGTCACG | |
| | CCCAATGCACCCGAGTTCTTCAGCCAAGCTCCTAAGCA | |
| | AGTTAGATGCAGCCGGGTCGATCGAGGAAATCAGGAAA | |
| | ATCAAGCGCCTTGCTCTAAATGGCTAATTACTACTGCCA | |
| | CACGTAGCGGGTCCCTGTCCACTCGGCATCACACGGAA | |
| | TCTGCACCGAGTTCCCCCCCGCAGACCCAAGGTCCAAC | |
| | TCTAGAAGCGGCAATCCTCTCTCGCTTCCTCAGCCCCAC | |
| | TGAATGATCGCGTAACCGTAATTAATCTAGCTACATTAA | |
| | GGATTAAGAAAAAATACGGGTAGAATTGGAGTGCCCCA | |
| | ATTGTGCCAAGATGGACTCATCTAGGACAATTGGGCTG | |
| | TACTTTGATTCTGCCCATTCTTCTAGCAACCTGTTAGCA | |
| | TTTCCGATCGTCCTACAAGACACAGGAGATGGGAAGAA | |
| | GCAAATCGCCCCGCAATATAGGATCCAGCGCCTTGACT | |
| | CGTGGACTGATAGTAAGGAAGACTCAGTATTCATCACC | |
| | ACCTATGGATTCATCTTTCAAGTTGGGAATGAGGAAGC | |
| | CACTGTCGGCATGATCGATGATAAACCCAAGCGCGAGT | |
| | TACTTTCCGCTGCGATGCTCTGCCTAGGAAGCGTCCCAA | |
| | ATACCGGAGACCTTGTTGAGCTGGCAAGGGCCTGTCTC | |
| | ACTATGATGGTCACATGCAAGAAGAGTGCAACTAATAC | |
| | TGAGAGAATGGTTTTCTCAGTAGTGCAGGCACCCCAAG | |
| | TGCTGCAAAGCTGTAGGGTTGTGGCAAATAAATACTCA | |
| | TCAGTGAATGCAGTCAAGCACGTGAAAGCGCCAGAGAA | |
| | GATCCCCGGGAGTGGAACCCTAGAATACAAGGTGAACT | |
| | TTGTCTCCTTGACTGTGGTACCGAAGAAGGATGTCTACA | |
| | AGATCCCAGCTGCAGTATTGAAGATTTCTGGCTCGAGTC | |
| | TGTACAATCTTGCGCTCAATGTCACTATTAATGTGGAGG | |
| | TAGACCCGAGGAGTCCTTTGGTTAAATCTCTGTCTAAGT | |
| | CTGACAGCGGATACTATGCTAACCTCTTCTTGCATATTG | |
| | GACTTATGACCACCGTAGATAGGAAGGGGAAGAAAGT | |
| | GACATTTGACAAGCTGGAAAAGAAAATAAGGAGCCTTG | |
| | ATCTATCTGTCGGGCTCAGTGATGTGCTCGGGCCTTCCG | |
| | TGTTGGTAAAAGCAAGAGGTGCACGGACTAAGCTTTTG | |
| | GCACCTTTCTTCTCTAGCAGTGGGACAGCCTGCTATCCC | |
| | ATAGCAAATGCTTCTCCTCAGGTGGCCAAGATACTCTG | |
| | GAGTCAAACCGCGTGCCTGCGGAGCGTTAAAATCATTA | |
| | TCCAAGCAGGTACCCAACGCGCTGTCGCAGTGACCGCT | |
| | GACCACGAGGTTACCTCTACTAAGCTGGAGAAGGGGCA | |
| | CACCCTTGCCAAATACAATCCTTTTAAGAAATAAGCTGC | |
| | GTCTCTGAGATTGCGCTCCGCCCACTCACCCAGATCATC | |
| | ATGACACAAAAAACTAATCTGTCTTGATTATTTACAGTT | |
| | AGTTTACCTGTCCATCAAGTTAGAAAAAACACGGGTAG | |
| | AAGATTCTGGATCCCGGTTGGCGCCCTCCAGGTGCAGG | |
| | ATGGGCTCCAGACCTTCTACCAAGAACCCAGCACCTAT | |
| | GATGCTGACTATCCGGGTCGCGCTGGTACTGAGTTGCAT | |
| | CTGCCCGGCAAACTCCATTGATGGCAGGCCTCTTGCAG | |
| | CTGCAGGAATTGTGGTTACAGGAGACAAAGCAGTCAAC | |
| | ATATACACCTCATCCCAGACAGGATCAATCATAGTTAA | |
| | GCTCCTCCCGAATCTGCCCAAGGATAAGGAGGCATGTG | |
| | CGAAAGCCCCCTTGGATGCATACAACAGGACATTGACC | |
| | ACTTTGCTCACCCCCCTTGGTGACTCTATCCGTAGGATA | |
| | CAAGAGTCTGTGACTACATCTGGAGGGGGGAGACAGGG | |
| | GCGCCTTATAGGCGCCATTATTGGCGGTGTGGCTCTTGG | |
| | GGTTGCAACTGCCGCACAAATAACAGCGGCCGCAGCTC | |
| | TGATACAAGCCAAACAAAATGCTGCCAACATCCTCCGA | |
| | CTTAAAGAGAGCATTGCCGCAACCAATGAGGCTGTGCA | |
| | TGAGGTCACTGACGGATTATCGCAACTAGCAGTGGCAG | |
| | TTGGGAAGATGCAGCAGTTTGTTAATGACCAATTTAAT | |
| | AAAACAGCTCAGGAATTAGACTGCATCAAAATTGCACA | |
| | GCAAGTTGGTGTAGAGCTCAACCTGTACCTAACCGAAT | |
| | TGACTACAGTATTCGGACCACAAATCACTTCACCTGCCT | |
| | TAAACAAGCTGACTATTCAGGCACTTTACAATCTAGCTG | |
| | GTGGGAATATGGATTACTTATTGACTAAGTTAGGTATA | |
| | GGGAACAATCAACTCAGCTCATTAATCGGTAGCGGCTT | |
| | AATCACCGGTAACCCTATTCTATACGACTCACAGACTCA | |
| | ACTCTTGGGTATACAGGTAACTCTACCTTCAGTCGGGAA | |
| | CCTAAATAATATGCGTGCCACCTACTTGGAAACCTTATC | |
| | CGTAAGCACAACCAGGGGATTTGCCTCGGCACTTGTCC | |
| | CAAAAGTGGTGACACAGGTCGGTTCTGTGATAGAAGAA | |
| | CTTGACACCTCATACTGTATAGAAACTGACTTAGATTTA | |
| | TATTGTACAAGAATAGTAACGTTCCCTATGTCCCCTGGT | |
| | ATTTACTCCTGCTTGAGCGGCAATACATCGGCCTGTATG | |
| | TACTCAAAGACCGAAGGCGCACTTACTACACCATATAT | |
| | GACTATCAAAGGCTCAGTCATCGCTAACTGCAAGATGA | |
| | CAACATGTAGATGTGTAAACCCCCCGGGTATCATATCG | |
| | CAAAACTATGGAGAAGCCGTGTCTCTAATAGATAAACA | |
| | ATCATGCAATGTTTTATCCTTAGGCGGGATAACTTTAAG | |
| | GCTCAGTGGGGAATTCGATGTAACTTATCAGAAGAATA | |
| | TCTCAATACAAGATTCTCAAGTAATAATAACAGGCAAT | |
| | CTTGATATCTCAACTGAGCTTGGGAATGTCAACAACTCG | |
| | ATCAGTAATGCTTTGAATAAGTTAGAGGAAAGCAACAG | |
| | AAAACTAGACAAAGTCAATGTCAAACTGACCAGCACAT | |
| | CTGCTCTCATTACCTATATCGTTTTGACTATCATATCTCT | |
| | TGTTTTTGGTATACTTAGCCTGATTCTAGCATGCTACCT | |
| | AATGTACAAGCAAAAGGCGCAACAAAAGACCTTATTAT | |
| | GGCTTGGGAATAATACCCTAGATCAGATGAGAGCCACT | |
| | ACAAAAATGTGAACACAGATGAGGAACGAAGGTTTCCC | |
| | TAATAGTAATTTGTGTGAAAGTTCTGGTAGTCTGTCAGT | |
| | TCGGAGAGTTAAGAAAAAACTACCGGTTGTAGATGACC | |
| | AAAGGACGATATACGGGTAGAACGGTAAGAGAGGCCG | |
| | CCCCTCAATTGCGAGCCAGACTTCACAACCTCCGTTCTA | |
| | CCGCTTCACCGACAACAGTCCTCAATCATGGACCGCGC | |
| | CGTTAGCCAAGTTGCGTTAGAGAATGATGAAAGAGAGG | |
| | CAAAAAATACATGGCGCTTGATATTCCGGATTGCAATC | |
| | TTATTCTTAACAGTAGTGACCTTGGCTATATCTGTAGCC | |
| | TCCCTTTTATATAGCATGGGGGCTAGCACACCTAGCGAT | |
| | CTTGTAGGCATACCGACTAGGATTTCCAGGGCAGAAGA | |
| | AAAGATTACATCTACACTTGGTTCCAATCAAGATGTAGT | |
| | AGATAGGATATATAAGCAAGTGGCCCTTGAGTCTCCAT | |
| | TGGCATTGTTAAATACTGAGACCACAATTATGAACGCA | |
| | ATAACATCTCTCTCTTATCAGATTAATGGAGCTGCAAAC | |
| | AACAGCGGGTGGGGGGCACCTATTCATGACCCAGATTA | |
| | TATAGGGGGGATAGGCAAAGAACTCATTGTAGATGATG | |
| | CTAGTGATGTCACATCATTCTATCCCTCTGCATTTCAAG | |
| | AACATCTGAATTTTATCCCGGCGCCTACTACAGGATCAG | |
| | GTTGCACTCGAATACCCTCATTTGACATGAGTGCTACCC | |
| | ATTACTGCTACACCCATAATGTAATATTGTCTGGATGCA | |
| | GAGATCACTCACACTCACATCAGTATTTAGCACTTGGTG | |
| | TGCTCCGGACATCTGCAACAGGGAGGGTATTCTTTTCTA | |
| | CTCTGCGTTCCATCAACCTGGACGACACCCAAAATCGG | |
| | AAGTCTTGCAGTGTGAGTGCAACTCCCCTGGGTTGTGAT | |
| | ATGCTGTGCTCGAAAGCCACGGAGACAGAGGAAGAAG | |
| | ATTATAACTCAGCTGTCCCTACGCGGATGGTACATGGG | |
| | AGGTTAGGGTTCGACGGCCAATATCACGAAAAGGACCT | |
| | AGATGTCACAACATTATTCGGGGACTGGGTGGCCAACT | |
| | ACCCAGGAGTAGGGGGTGGATCTTTTATTGACAGCCGC | |
| | GTATGGTTCTCAGTCTACGGAGGGTTAAAACCCAATAC | |
| | ACCCAGTGACACTGTACAGGAAGGGAAATATGTGATAT | |
| | ACAAGCGATACAATGACACATGCCCAGATGAGCAAGAC | |
| | TACCAGATTCGAATGGCCAAGTCTTCGTATAAGCCTGG | |
| | ACGGTTTGGTGGGAAACGCATACAGCAGGCTATCTTAT | |
| | CTATCAAAGTGTCAACATCCTTAGGCGAAGACCCGGTA | |
| | CTGACTGTACCGCCCAACACAGTCACACTCATGGGGGC | |
| | CGAAGGCAGAATTCTCACAGTAGGGACATCCCATTTCT | |
| | TGTATCAGCGAGGGTCATCATACTTCTCTCCCGCGTTAT | |
| | TATATCCTATGACAGTCAGCGACAAAACAGCCACTCTT | |
| | CATAGTCCTTATACATTCAATGCCTTCACTCGGCCAGGT | |
| | AGTATCCCTTGCCAGGCTTCAGCAAGATGCCCCAACTC | |
| | GTGTGTTACTGGAGTCTATACAGATCCATATCCCCTAAT | |
| | CTTCTATAGAAACCACACCTTGCGAGGGGTATTCGGGA | |
| | CAATGCTTGATGGTGAACAAGCAAGACTTAACCCTGCG | |
| | TCTGCAGTATTCGATAGCACATCCCGCAGTCGCATAACT | |
| | CGAGTGAGTTCAAGCAGCATCAAAGCAGCATACACAAC | |
| | ATCAACTTGTTTTAAAGTGGTCAAGACCAATAAGACCT | |
| | ATTGTCTCAGCATTGCTGAAATATCTAATACTCTCTTCG | |
| | GAGAATTCAGAATCGTCCCGTTACTAGTTGAGATCCTCA | |
| | AAGATGACGGGGTTAGAGAAGCCAGGTCTGGCTAGTTG | |
| | AGTCAACTATGAAAGAGTTGGAAAGATGGCATTGTATC | |
| | ACCTATCTTCTGCGACATCAAGAATCAAACCGAATGCC | |
| | GGCGCGTGCTCGAATTCCATGTCGCCAGTTGACCACAA | |
| | TCAGCCAGTGCTCATGCGATCAGATTAAGCCTTGTCAAT | |
| | AGTCTCTTGATTAAGAAAAAATGTAAGTGGCAATGAGA | |
| | TACAAGGCAAAACAGCTCACGGTAAATAATACGGGTAG | |
| | GACATGGCGAGCTCCGGTCCTGAAAGGGCAGAGCATCA | |
| | GATTATCCTACCAGAGTCACACCTGTCTTCACCATTGGT | |
| | CAAGCACAAACTACTCTATTATTGGAAATTAACTGGGC | |
| | TACCGCTTCCTGATGAATGTGACTTCGACCACCTCATTC | |
| | TCAGCCGACAATGGAAAAAAATACTTGAATCGGCCTCT | |
| | CCTGATACTGAGAGAATGATAAAACTCGGAAGGGCAGT | |
| | ACACCAAACTCTTAACCACAATTCCAGAATAACCGGAG | |
| | TACTCCACCCCAGGTGTTTAGAAGAACTGGCTAATATTG | |
| | AGGTCCCTGATTCAACCAACAAATTTCGGAAGATTGAG | |
| | AAGAAGATCCAAATTCACAACACGAGATATGGAGAACT | |
| | GTTCACAAGGCTGTGTACGCATATAGAGAAGAAACTGC | |
| | TGGGGTCATCTTGGTCTAACAATGTCCCCCGGTCAGAG | |
| | GAGTTCAGCAGCATTCGTACGGATCCGGCATTCTGGTTT | |
| | CACTCAAAATGGTCCACAGCCAAGTTTGCATGGCTCCA | |
| | TATAAAACAGATCCAGAGGCATCTGATTGTGGCAGCTA | |
| | GGACAAGGTCTGCGGCCAACAAATTGGTGATGCTAACC | |
| | CATAAGGTAGGCCAAGTCTTTGTCACTCCTGAACTTGTT | |
| | GTTGTGACGCATACGAATGAGAACAAGTTCACATGTCT | |
| | TACCCAGGAACTTGTATTGATGTATGCAGATATGATGG | |
| | AGGGCAGAGATATGGTCAACATAATATCAACCACGGCG | |
| | GTGCATCTCAGAAGCTTATCAGAGAAAATTGATGACAT | |
| | TTTGCGGTTAATAGACGCTCTGGCAAAAGACTTGGGTA | |
| | ATCAAGTCTACGATGTTGTATCACTAATGGAGGGATTTG | |
| | CATACGGAGCTGTCCAGCTACTCGAGCCGTCAGGTACA | |
| | TTTGCGGGAGATTTCTTCGCATTCAACCTGCAGGAGCTT | |
| | AAAGACATTCTAATTGGCCTCCTCCCCAATGATATAGCA | |
| | GAATCCGTGACTCATGCAATCGCTACTGTATTCTCTGGT | |
| | TTAGAACAGAATCAAGCAGCTGAGATGTTGTGCCTGTT | |
| | GCGTCTGTGGGGTCACCCACTGCTTGAGTCCCGTATTGC | |
| | AGCAAAGGCAGTCAGGAGCCAAATGTGCGCACCGAAA | |
| | ATGGTAGACTTTGATATGATCCTTCAGGTACTGTCTTTC | |
| | TTCAAGGGAACAATCATCAACGGATACAGAAAGAAGA | |
| | ATGCAGGTGTGTGGCCGCGAGTCAAAGTGGATACAATA | |
| | TATGGGAAGGTCATTGGGCAACTACATGCAGATTCAGC | |
| | AGAGATTTCACACGATATCATGTTGAGAGAGTATAAGA | |
| | GTTTATCTGCACTTGAATTTGAGCCATGTATAGAATACG | |
| | ACCCTGTCACTAACCTGAGCATGTTCCTAAAAGACAAG | |
| | GCAATCGCACACCCCAACGATAATTGGCTTGCCTCGTTT | |
| | AGGCGGAACCTTCTCTCCGAAGACCAGAAGAAACATGT | |
| | AAAGGAAGCGACTTCGACTAACCGCCTCTTGATAGAGT | |
| | TTTTAGAGTCAAATGATTTTGATCCATATAAAGAGATGG | |
| | AATATCTGACGACCCTTGAGTACCTTAGAGATGACAAT | |
| | GTGGCAGTATCATACTCGCTCAAAGAGAAGGAAGTGAA | |
| | AGTTAATGGACGGATCTTCGCTAAGCTGACAAAGAAGT | |
| | TAAGGAACTGTCAGGTGATGGCGGAAGGGATCCTAGCC | |
| | GATCAGATTGCACCTTTCTTTCAGGGAAATGGAGTCATT | |
| | CAGGATAGCATATCCTTGACCAAGAGTATGCTAGCGAT | |
| | GAGTCAACTGTCTTTTAACAGCAATAAGAAACGTATCA | |
| | CTGACTGTAAAGAAAGAGTATGTTCAAACCGCAATCAT | |
| | GATCCGAAAAGCAAGAACCGTCGGAGAGTTGCAACCTT | |
| | CATAACAACTGACCTGCAAAAGTACTGTCTTAATTGGA | |
| | GATATCAGACGATCAAATTGTTCGCTCATGCCATCAATC | |
| | AGTTGATGGGCCTACCTCATTTCTTCGAGTGGATTCACC | |
| | TAAGACTGATGGACACTACGATGTTCGTAGGAGACCCT | |
| | TTCAATCCTCCAAGTGACCCTACTGACTGTGACCTCTCA | |
| | AGAGTCCCTAATGATGACATATATATTGTCAGTGCCAG | |
| | AGGGGGTATCGAAGGATTATGCCAGAAGCTATGGACAA | |
| | TGATCTCAATTGCTGCAATCCAACTTGCTGCAGCTAGAT | |
| | CGCATTGTCGTGTTGCCTGTATGGTACAGGGTGATAATC | |
| | AAGTAATAGCAGTAACGAGAGAGGTAAGATCAGATGA | |
| | CTCTCCGGAGATGGTGTTGACACAGTTGCATCAAGCCA | |
| | GTGATAATTTCTTCAAGGAATTAATCCATGTCAATCATT | |
| | TGATTGGCCATAATTTGAAGGATCGTGAAACCATCAGG | |
| | TCAGACACATTCTTCATATACAGCAAACGAATCTTCAA | |
| | AGATGGAGCAATCCTCAGTCAAGTCCTCAAAAATTCAT | |
| | CTAAATTAGTGCTAGTGTCAGGTGATCTCAGTGAAAAC | |
| | ACCGTAATGTCCTGTGCCAACATTGCCTCTACTGTAGCA | |
| | CGGCTATGCGAGAACGGGCTTCCCAAAGACTTCTGTTA | |
| | CTATTTAAACTATATAATGAGTTGTGTGCAGACATACTT | |
| | TGACTCTGAGTTCTCCATCACCAACAATTCGCACCCCGA | |
| | TCTTAATCAGTCGTGGATTGAGGACATCTCTTTTGTGCA | |
| | CTCATATGTTCTGACTCCTGCCCAATTAGGGGGACTGAG | |
| | TAACCTTCAATACTCAAGGCTCTACACTAGAAATATCG | |
| | GTGACCCGGGGACTACTGCTTTTGCAGAGATCAAGCGA | |
| | CTAGAAGCAGTGGGACTACTGAGTCCTAACATTAGGAC | |
| | TAATATCTTAACTAGGCCGCCTGGGAATGGAGATTGGG | |
| | CCAGTCTGTGCAACGACCCATACTCTTTCAATTTTGAGA | |
| | CTGTTGCAAGCCCAAACATTGTTCTTAAGAAACATACG | |
| | CAAAGAGTCCTATTTGAAACTTGTTCAAATCCCTTATTG | |
| | TCTGGAGTGCACACAGAGGATAATGAGGCAGAAGAGA | |
| | AGGCATTGGCTGAATTCTTGCTTAATCAAGAGGTGATTC | |
| | ATCCCCGCGTTGCGCATGCCATCATGGAGGCAAGCTCT | |
| | GTAGGTAGGAGAAAGCAAATTCAAGGGCTTGTTGACAC | |
| | AACAAACACTGTAATTAAGATTGCGCTTACTAGGAGGC | |
| | CATTAGGCATCAAGAGGCTGATGCGGATAGTCAATTAT | |
| | TCTAGCATGCATGCAATGCTGTTTAGAGACGATGTTTTT | |
| | TCCTCTAGTAGATCCAACCACCCCTTAGTCTCTTCTAAT | |
| | ATGTGTTCTCTGACACTGGCAGACTATGCACGGAATAG | |
| | AAGCTGGTCACCTTTGACGGGAGGCAGGAAAATACTGG | |
| | GTGTATCTAATCCTGATACGATAGAACTCGTAGAGGGT | |
| | GAGATTCTTAGTGTAAGCGGAGGGTGTACAAGATGTGA | |
| | CAGCGGAGATGAACAATTTACTTGGTTCCATCTTCCAAG | |
| | CAATATAGAATTGACCGATGACACCAGCAAGAATCCTC | |
| | CGATGAGGGTACCATATCTCGGGTCAAAGACACAGGAG | |
| | AGGAGAGCTGCCTCACTTGCGAAAATAGCTCATATGTC | |
| | GCCACATGTGAAGGCTGCCCTAAGGGCATCATCCGTGT | |
| | TGATCTGGGCTTATGGGGATAATGAAGTAAATTGGACT | |
| | GCTGCTCTTACGATTGCAAAATCTCGGTGTAATGTAAAC | |
| | TTAGAGTATCTTCGGTTACTGTCCCCTTTACCCACGGCT | |
| | GGGAATCTTCAACATAGACTAGATGATGGTATAACTCA | |
| | GATGACATTCACCCCTGCATCTCTCTACAGGGTGTCACC | |
| | TTACATTCACATATCCAATGATTCTCAAAGGCTGTTCAC | |
| | TGAAGAAGGAGTCAAAGAGGGGAATGTGGTTTACCAAC | |
| | AGATCATGCTCTTGGGTTTATCTCTAATCGAATCGATCT | |
| | TTCCAATGACAACAACCAGAACATATGATGAGATCACA | |
| | CTGCACCTACATAGTAAATTTAGTTGCTGTATCAGGGAA | |
| | GCACCTGTTGCGGTTCCTTTCGAGCTACTTGGGGTGGCA | |
| | CCGGAACTGAGGACAGTGACCTCAAATAAGTTTATGTA | |
| | TGATCCTAGCCCTGTATCGGAGGGAGACTTTGCGAGAC | |
| | TTGACTTAGCTATCTTCAAGAGTTATGAGCTTAATCTGG | |
| | AGTCATATCCCACGATAGAGCTAATGAACATTCTTTCAA | |
| | TATCCAGCGGGAAGTTGATTGGCCAGTCTGTGGTTTCTT | |
| | ATGATGAAGATACCTCCATAAAGAATGATGCCATAATA | |
| | GTGTATGACAATACCCGAAATTGGATCAGTGAAGCTCA | |
| | GAATTCAGATGTGGTCCGCCTATTTGAATATGCAGCACT | |
| | TGAAGTGCTCCTCGACTGTTCTTACCAACTCTATTACCT | |
| | GAGAGTAAGAGACCTAGACAATATTGTCTTATATATGG | |
| | GTGATTTATACAAGAATATGCCAGGAATTCTACTTTCCA | |
| | ACATTGCAGCTACAATATCTCATCCTGTCATTCATTCAA | |
| | GGTTACATGCAGTGGGCCTGGTCAACCATGACGGATCA | |
| | CACCAACTTGCAGATACGGATTTTATCGAAATGTCTGCA | |
| | AAACTGTTAGTATCTTGCACCCGACGTGTGATCTCCGGC | |
| | TTATATTCAGGAAATAAGTATGATCTGCTGTTCCCATCT | |
| | GTCTTAGATGATAACCTGAATGAGAAGATGCTTCAGCT | |
| | GATATCCCGGTTATGCTGTCTGTACACGGTACTCTTTGC | |
| | TACAACAAGAGAAATCCCGAAAATAAGAGGCTTAACTG | |
| | CAGAAGAGAAATGTTCAATACTCACTGAGTATTTACTG | |
| | TCGGATGCTGTGAAACCATTACTTAGCCCCGATCAAGT | |
| | GAGCTCTATCATGTCTCCTAACATAATTACATTCCCAGC | |
| | TAATCTGTACTACATGTCTCGGAAGAGCCTCAATTTGAT | |
| | CAGGGAAAGGGAGGACAGGGATACTATCCTGGCGTTGT | |
| | TGTTCCCCCAAGAGCCATTATTAGAGTTCCCTTCTGTGC | |
| | AAGATATTGGTGCTCGAGTGAAAGATCCATTCACCCGA | |
| | CAACCTGCGGCATTTTTGCAAGAGTTAGATTTGAGTGCT | |
| | CCAGCAAGGTATGACGCATTCACACTTAGTCAGATTCA | |
| | TCCTGAACTCACATCTCCAAATCCGGAGGAAGACTACT | |
| | TAGTACGATACTTGTTCAGAGGGATAGGGACTGCATCT | |
| | TCCTCTTGGTATAAGGCATCCCATCTCCTTTCTGTACCC | |
| | GAGGTAAGATGTGCAAGACACGGGAACTCCTTATACTT | |
| | GGCTGAAGGAAGCGGAGCCATCATGAGTCTTCTTGAAC | |
| | TGCATGTACCACATGAAACTATCTATTACAATACGCTCT | |
| | TTTCAAATGAGATGAACCCCCCGCAACGACATTTCGGG | |
| | CCGACCCCAACTCAGTTTTTGAATTCGGTTGTTTATAGG | |
| | AATCTACAGGCGGAGGTAACATGCAAGGATGGATTTGT | |
| | CCAAGAGTTCCGTCCATTATGGAGAGAAAATACAGAGG | |
| | AAAGTGACCTGACCTCAGATAAAGCAGTGGGGTATATT | |
| | ACATCTGCAGTACCCTACAGATCTGTATCATTGCTGCAT | |
| | TGTGACATTGAAATTCCTCCAGGGTCCAATCAAAGCTTA | |
| | CTAGATCAACTAGCTATCAATTTATCTCTGATTGCCATG | |
| | CATTCTGTAAGGGAGGGCGGGGTAGTAATCATCAAAGT | |
| | GTTGTATGCAATGGGATACTACTTTCATCTACTCATGAA | |
| | CTTGTTTGCTCCGTGTTCCACAAAAGGATATATTCTCTC | |
| | TAATGGTTATGCATGTCGAGGGGATATGGAGTGTTACC | |
| | TGGTATTTGTCATGGGTTACCTGGGCGGGCCTACATTTG | |
| | TACATGAGGTGGTGAGGATGGCAAAAACTCTGGTGCAG | |
| | CGGCACGGTACGCTTTTGTCTAAATCAGATGAGATCAC | |
| | ACTGACCAGGTTATTCACCTCACAGCGGCAGCGTGTGA | |
| | CAGACATCCTATCCAGTCCTTTACCAAGATTAATAAAGT | |
| | ACTTGAGGAAGAATATTGACACTGCGCTGATTGAAGCC | |
| | GGGGGACAGCCCGTCCGTCCATTCTGTGCGGAGAGTCT | |
| | GGTGAGCACGCTAGCGAACATAACTCAGATAACCCAGA | |
| | TCATCGCTAGTCACATTGACACAGTCATCCGGTCTGTGA | |
| | TATATATGGAAGCTGAGGGTGATCTCGCTGACACAGTA | |
| | TTTCTATTTACCCCTTACAATCTCTCTACTGACGGGAAA | |
| | AAGAGGACATCACTTAAACAGTGCACGAGACAGATCCT | |
| | AGAGGTTACAATACTAGGTCTTAGAGTCGAAAATCTCA | |
| | ATAAAATAGGCGATATAATCAGCCTAGTGCTTAAAGGC | |
| | ATGATCTCCATGGAGGACCTTATCCCACTAAGGACATA | |
| | CTTGAAGCATAGTACCTGCCCTAAATATTTGAAGGCTGT | |
| | CCTAGGTATTACCAAACTCAAAGAAATGTTTACAGACA | |
| | CTTCTGTACTGTACTTGACTCGTGCTCAACAAAAATTCT | |
| | ACATGAAAACTATAGGCAATGCAGTCAAAGGATATTAC | |
| | AGTAACTGTGACTCCTAACGAAAATCACATATTAATAG | |
| | GCTCCTTTTTTGGCCAATTGTATTCTTGTTGATTTAATTA | |
| | TATTATGTTAGAAAAAAGTTGAACTCTGACTCCTTAGGA | |
| | CTCGAATTCGAACTCAAATAAATGTCTTTAAAAAAGGT | |
| | TGCGCACAATTATTCTTGAGTGTAGTCTCGTCATTCACC | |
| | AAATCTTTGTTTGGT | |
| cDNA of | accaaacagagaatccgtgagttacgataaaaggcgaaggagcaattgaagtcgcacggg | SEQ ID |
| genomic | tagaaggtgtgaatctcgagtgcgagcccgaagcacaaactegagaaagccttctgccaac | NO: 3 |
| sequence of | atgtcttccgtatttgatgagtacgaacagctcctcgcggctcagactcgccccaatggagct | |
| NDV strain | catggagggggagaaaaagggagtaccttaaaagtagacgtcccggtattcactcttaaca | |
| LaSota | gtgatgacccagaagatagatggagctttgtggtattctgcctccggattgctgttagcgaag | |
| (L289A | atgccaacaaaccactcaggcaaggtgctctcatatctcttttatgctcccactcacaggtaat | |
| mutation) | gaggaaccatgttgcccttgcagggaaacagaatgaagccacattggccgtgcttgagattg | |
| | atggctttgccaacggcacgccccagttcaacaataggagtggagtgtctgaagagagagc | |
| | acagagatttgcgatgatagcaggatctctccctcgggcatgcagcaacggaaccccgttc | |
| | gtcacagccggggccgaagatgatgcaccagaagacatcaccgataccctggagaggat | |
| | cctctctatccaggctcaagtatgggtcacagtagcaaaagccatgactgcgtatgagactg | |
| | cagatgagtcggaaacaaggcgaatcaataagtatatgcagcaaggcagggtccaaaaga | |
| | aatacatcctctaccccgtatgcaggagcacaatccaactcacgatcagacagtctcttgcag | |
| | tccgcatctttttggttagcgagctcaagagaggccgcaacacggcaggtggtacctctactt | |
| | attataacctggtaggggacgtagactcatacatcaggaataccgggcttactgcattcttctt | |
| | gacactcaagtacggaatcaacaccaagacatcagcccttgcacttagtagcctctcaggcg | |
| | acatccagaagatgaagcagctcatgcgtttgtatcggatgaaaggagataatgcgccgtac | |
| | atgacattacttggtgatagtgaccagatgagctttgcgcctgccgagtatgcacaactttact | |
| | cctttgccatgggtatggcatcagtcctagataaaggtactgggaaataccaatttgccaggg | |
| | actttatgagcacatcattctggagacttggagtagagtacgctcaggctcagggaagtagc | |
| | attaacgaggatatggctgccgagctaaagctaaccccagcagcaaggaggggcctggca | |
| | gctgctgcccaacgggtctccgaggagaccagcagcatagacatgcctactcaacaagtc | |
| | ggagtcctcactgggcttagcgagggggggtcccaagctctacaaggcggatcgaataga | |
| | tcgcaagggcaaccagaagccggggatggggagacccaattcctggatctgatgagagc | |
| | ggtagcaaatagcatgagggaggcgccaaactctgcacagggcactccccaatcggggc | |
| | ctcccccaactcctgggccatcccaagataacgacaccgactgggggtattgatggacaaa | |
| | acccagcctgcttccacaaaaacatcccaatgccctcacccgtagtcgacccctcgatttgc | |
| | ggctctatatgaccacaccctcaaacaaacatccccctctttcctccctccccctgctgtacaa | |
| | ctacgtacgccctagataccacaggcacaatgcggctcactaacaatcaaaacagagccga | |
| | gggaattagaaaaaagtacgggtagaagagggatattcagagatcagggcaagtctcccg | |
| | agtctctgctctctcctctacctgatagaccaggacaaacatggccacctttacagatgcaga | |
| | gatcgacgagctatttgagacaagtggaactgtcattgacaacataattacagcccagggta | |
| | aaccagcagagactgttggaaggagtgcaatcccacaaggcaagaccaaggtgctgagc | |
| | gcagcatgggagaagcatgggagcatccagccaccggccagtcaagacaaccccgatcg | |
| | acaggacagatctgacaaacaaccatccacacccgagcaaacgaccccgcatgacagcc | |
| | cgccggccacatccgccgaccagccccccacccaggccacagacgaagccgtcgacac | |
| | acagctcaggaccggagcaagcaactctctgctgttgatgcttgacaagctcagcaataaat | |
| | cgtccaatgctaaaaagggcccatggtcgagcccccaagaggggaatcaccaacgtccga | |
| | ctcaacagcaggggagtcaacccagtcgcggaaacagtcaggaaagaccgcagaaccaa | |
| | gtcaaggccgcccctggaaaccagggcacagacgtgaacacagcatatcatggacaatgg | |
| | gaggagtcacaactatcagctggtgcaacccctcatgctctccgatcaaggcagagccaag | |
| | acaatacccttgtatctgcggatcatgtccagccacctgtagactttgtgcaagcgatgatgtc | |
| | tatgatggaggcgatatcacagagagtaagtaaggttgactatcagctagatcttgtcttgaaa | |
| | cagacatcctccatccctatgatgcggtccgaaatccaacagctgaaaacatctgttgcagtc | |
| | atggaagccaacttgggaatgatgaagattctggatcccggttgtgccaacatttcatctctga | |
| | gtgatctacgggcagttgcccgatctcacccggttttagtttcaggccctggagacccctctc | |
| | cctatgtgacacaaggaggcgaaatggcacttaataaactttcgcaaccagtgccacatcca | |
| | tctgaattgattaaacccgccactgcatgcgggcctgatataggagtggaaaaggacactgt | |
| | ccgtgcattgatcatgtcacgcccaatgcacccgagttcttcagccaagctcctaagcaagtt | |
| | agatgcagccgggtcgatcgaggaaatcaggaaaatcaagcgccttgctctaaatggctaa | |
| | ttactactgccacacgtagcgggtccctgtccactcggcatcacacggaatctgcaccgagtt | |
| | cccccccgcggacccaaggtccaactctccaagcggcaatcctctctcgcttcctcagcccc | |
| | actgaatgatcgcgtaaccgtaattaatctagctacatttaagattaagaaaaaatacgggtag | |
| | aattggagtgccccaattgtgccaagatggactcatctaggacaattgggctgtactttgattct | |
| | gcccattcttctagcaacctgttagcatttccgatcgtcctacaagacacaggagatgggaag | |
| | aagcaaatcgccccgcaatataggatccagcgccttgacttgtggactgatagtaaggagg | |
| | actcagtattcatcaccacctatggattcatctttcaagttgggaatgaagaagccaccgtcgg | |
| | catgatcgatgataaacccaagcgcgagttactttccgctgcgatgctctgcctaggaagcgt | |
| | cccaaataccggagaccttattgagctggcaagggcctgtctcactatgatagtcacatgcaa | |
| | gaagagtgcaactaatactgagagaatggttttctcagtagtgcaggcaccccaagtgctgc | |
| | aaagctgtagggttgtggcaaacaaatactcatcagtgaatgcagtcaagcacgtgaaagc | |
| | gccagagaagattcccgggagtggaaccctagaatacaaggtgaactttgtctccttgactgt | |
| | ggtaccgaagagggatgtctacaagatcccagctgcagtattgaaggtttctggctcgagtct | |
| | gtacaatcttgcgctcaatgtcactattaatgtggaggtagacccgaggagtcctttggttaaat | |
| | ctctgtctaagtctgacagcggatactatgctaacctcttcttgcatattggacttatgaccactg | |
| | tagataggaaggggaagaaagtgacatttgacaagctggaaaagaaaataaggagccttg | |
| | atctatctgtcgggctcagtgatgtgctcgggccttccgtgttggtaaaagcaagaggtgcac | |
| | ggactaagcttttggcacctttcttctctagcagtgggacagcctgctatcccatagcaaatgc | |
| | ttctcctcaggtggccaagatactctggagtcaaaccgcgtgcctgcggagcgttaaaatcat | |
| | tatccaagcaggtacccaacgcgctgtcgcagtgaccgccgaccacgaggttacctctact | |
| | aagctggagaaggggcacacccttgccaaatacaatccttttaagaaataagctgcgtctctg | |
| | agattgcgctccgcccactcacccagatcatcatgacacaaaaaactaatctgtcttgattattt | |
| | acagttagtttacctgtctatcaagttagaaaaaacacgggtagaagattctggatcccggttg | |
| | gcgccctccaggtgcaagatgggctccagaccttctaccaagaacccagcacctatgatgc | |
| | tgactatccgggttgcgctggtactgagttgcatctgtccggcaaactccattgatggcaggc | |
| | ctcttgcagctgcaggaattgtggttacaggagacaaagccgtcaacatatacacctcatccc | |
| | agacaggatcaatcatagttaagctcctcccgaatctgcccaaggataaggaggcatgtgcg | |
| | aaagcccccttggatgcatacaacaggacattgaccactttgctcaccccccttggtgactct | |
| | atccgtaggatacaagagtctgtgactacatctggaggggggagacaggggcgccttatag | |
| | gcgccattattggcggtgtggctcttggggttgcaactgccgcacaaataacagcggccgc | |
| | agctctgatacaagccaaacaaaatgctgccaacatcctccgacttaaagagagcattgccg | |
| | caaccaatgaggctgtgcatgaggtcactgacggattatcgcaactagcagtggcagttgg | |
| | gaagatgcagcagtttgttaatgaccaatttaataaaacagctcaggaattagactgcatcaaa | |
| | attgcacagcaagttggtgtagagctcaacctgtacctaaccgaattgactacagtattcgga | |
| | ccacaaatcacttcacctgctttaaacaagctgactattcaggcactttacaatctagctggtgg | |
| | aaatatggattacttattgactaagttaggtgtagggaacaatcaactcagctcattaatcggta | |
| | gcggcttaatcaccggtaaccctattctatacgactcacagactcaactcttgggtatacaggt | |
| | aactgccccttcagtcgggaacctaaataatatgcgtgccacctacttggaaaccttatccgta | |
| | agcacaaccaggggatttgcctcggcacttgtcccaaaagtggtgacacaggtcggttctgt | |
| | gatagaagaacttgacacctcatactgtatagaaactgacttagatttatattgtacaagaatag | |
| | taacgttccctatgtcccctggtatttattcctgcttgagcggcaatacgtcggcctgtatgtact | |
| | caaagaccgaaggcgcacttactacaccatacatgactatcaaaggttcagtcatcgccaac | |
| | tgcaagatgacaacatgtagatgtgtaaaccccccgggtatcatatcgcaaaactatggaga | |
| | agccgtgtctctaatagataaacaatcatgcaatgttttatccttaggcgggataactttaaggc | |
| | tcagtggggaattcgatgtaacttatcagaagaatatctcaatacaagattctcaagtaataata | |
| | acaggcaatcttgatatctcaactgagcttgggaatgtcaacaactcgatcagtaatgctttga | |
| | ataagttagaggaaagcaacagaaaactagacaaagtcaatgtcaaactgactagcacatct | |
| | gctctcattacctatatcgttttgactatcatatctcttgtttttggtatacttagcctgattctag | |
| | catgctacctaatgtacaagcaaaaggcgcaacaaaagaccttattatggcttgggaataatactc | |
| | tagatcagatgagagccactacaaaaatgtgaacacagatgaggaacgaaggtttccctaat | |
| | agtaatttgtgtgaaagttctggtagtctgtcagttcagagagttaagaaaaaactaccggttgt | |
| | agatgaccaaaggacgatatacgggtagaacggtaagagaggccgcccctcaattgcgag | |
| | ccaggcttcacaacctccgttctaccgcttcaccgacaacagtcctcaatcatggaccgcgc | |
| | cgttagccaagttgcgttagagaatgatgaaagagaggcaaaaaatacatggcgcttgatatt | |
| | ccggattgcaatcttattcttaacagtagtgaccttggctatatctgtagcctcccttttatatagc | |
| | atgggggctagcacacctagcgatcttgtaggcataccgactaggatttccagggcagaag | |
| | aaaagattacatctacacttggttccaatcaagatgtagtagataggatatataagcaagtggc | |
| | ccttgagtctccgttggcattgttaaatactgagaccacaattatgaacgcaataacatctctct | |
| | cttatcagattaatggagctgcaaacaacagtgggggggggcacctatccatgacccagat | |
| | tatataggggggataggcaaagaactcattgtagatgatgctagtgatgtcacatcattctatc | |
| | cctctgcatttcaagaacatctgaattttatcccggcgcctactacaggatcaggttgcactcg | |
| | aataccctcatttgacatgagtgctacccattactgctacacccataatgtaatattgtctggatg | |
| | cagagatcactcacattcatatcagtatttagcacttggtgtgctccggacatctgcaacaggg | |
| | agggtattcttttctactctgcgttccatcaacctggacgacacccaaaatcggaagtcttgca | |
| | gtgtgagtgcaactcccctgggttgtgatatgctgtgctcgaaagtcacggagacagaggaa | |
| | gaagattataactcagctgtccctacgcggatggtacatgggaggttagggttcgacggcca | |
| | gtaccacgaaaaggacctagatgtcacaacattattcggggactgggtggccaactaccca | |
| | ggagtagggggtggatcttttattgacagccgcgtatggttctcagtctacggagggttaaaa | |
| | cccaattcacccagtgacactgtacaggaagggaaatatgtgatatacaagcgatacaatga | |
| | cacatgcccagatgagcaagactaccagattcgaatggccaagtcttcgtataagcctggac | |
| | ggtttggtgggaaacgcatacagcaggctatcttatctatcaaggtgtcaacatccttaggcg | |
| | aagacccggtactgactgtaccgcccaacacagtcacactcatgggggccgaaggcagaa | |
| | ttctcacagtagggacatctcatttcttgtatcaacgagggtcatcatacttctctcccgcgttatt | |
| | atatcctatgacagtcagcaacaaaacagccactcttcatagtccttatacattcaatgccttca | |
| | ctcggccaggtagtatcccttgccaggcttcagcaagatgccccaactcgtgtgttactggag | |
| | tctatacagatccatatcccctaatcttctatagaaaccacaccttgcgaggggtattcgggac | |
| | aatgcttgatggtgtacaagcaagacttaaccctgcgtctgcagtattcgatagcacatcccg | |
| | cagtcgcattactcgagtgagttcaagcagtaccaaagcagcatacacaacatcaacttgtttt | |
| | aaagtggtcaagactaataagacctattgtctcagcattgctgaaatatctaatactctcttcgg | |
| | agaattcagaatcgtcccgttactagttgagatcctcaaagatgacggggttagagaagcca | |
| | ggtctggctagttgagtcaattataaaggagttggaaagatggcattgtatcacctatcttctgc | |
| | gacatcaagaatcaaaccgaatgccggcgcgtgctcgaattccatgttgccagttgaccaca | |
| | atcagccagtgctcatgcgatcagattaagccttgtcaatagtctcttgattaagaaaaaatgta | |
| | agtggcaatgagatacaaggcaaaacagctcatggttaacaatacgggtaggacatggcga | |
| | gctccggtcctgaaagggcagagcatcagattatcctaccagagtcacacctgtcttcaccat | |
| | tggtcaagcacaaactactctattactggaaattaactgggctaccgcttcctgatgaatgtga | |
| | cttcgaccacctcattctcagccgacaatggaaaaaaatacttgaatcggcctctcctgatact | |
| | gagagaatgataaaactcggaagggcagtacaccaaactcttaaccacaattccagaataa | |
| | ccggagtgctccaccccaggtgtttagaagaactggctaatattgaggtcccagattcaacc | |
| | aacaaatttcggaagattgagaagaagatccaaattcacaacacgagatatggagaactgtt | |
| | cacaaggctgtgtacgcatatagagaagaaactgctggggtcatcttggtctaacaatgtccc | |
| | ccggtcagaggagttcagcagcattcgtacggatccggcattctggtttcactcaaaatggtc | |
| | cacagccaagtttgcatggctccatataaaacagatccagaggcatctgatggtggcagcta | |
| | ggacaaggtctgcggccaacaaattggtgatgctaacccataaggtaggccaagtctttgtc | |
| | actcctgaacttgtcgttgtgacgcatacgaatgagaacaagttcacatgtcttacccaggaa | |
| | cttgtattgatgtatgcagatatgatggagggcagagatatggtcaacataatatcaaccacg | |
| | gcggtgcatctcagaagcttatcagagaaaattgatgacattttgcggttaatagacgctctgg | |
| | caaaagacttgggtaatcaagtctacgatgttgtatcactaatggagggatttgcatacggag | |
| | ctgtccagctactcgagccgtcaggtacatttgcaggagatttcttcgcattcaacctgcagga | |
| | gcttaaagacattctaattggcctcctccccaatgatatagcagaatccgtgactcatgcaatc | |
| | gctactgtattctctggtttagaacagaatcaagcagctgagatgttgtgtctgttgcgtctgtg | |
| | gggtcacccactgcttgagtcccgtattgcagcaaaggcagtcaggagccaaatgtgcgca | |
| | ccgaaaatggtagactttgatatgatccttcaggtactgtctttcttcaagggaacaatcatcaa | |
| | cgggtacagaaagaagaatgcaggtgtgtggccgcgagtcaaagtggatacaatatatgg | |
| | gaaggtcattgggcaactacatgcagattcagcagagatttcacacgatatcatgttgagaga | |
| | gtataagagtttatctgcacttgaatttgagccatgtatagaatatgaccctgtcaccaacctga | |
| | gcatgttcctaaaagacaaggcaatcgcacaccccaacgataattggcttgcctcgtttaggc | |
| | ggaaccttctctccgaagaccagaagaaacatgtaaaagaagcaacttcgactaatcgcctc | |
| | ttgatagagtttttagagtcaaatgattttgatccatataaagagatggaatatctgacgaccctt | |
| | gagtaccttagagatgacaatgtggcagtatcatactcgctcaaggagaaggaagtgaaagt | |
| | taatggacggatcttcgctaagctgacaaagaagttaaggaactgtcaggtgatggcggaa | |
| | gggatcctagccgatcagattgcacctttctttcagggaaatggagtcattcaggatagcatat | |
| | ccttgaccaagagtatgctagcgatgagtcaactgtcttttaacagcaataagaaacgtatcac | |
| | tgactgtaaagaaagagtatcttcaaaccgcaatcatgatccgaaaagcaagaaccgtcgga | |
| | gagttgcaaccttcataacaactgacctgcaaaagtactgtcttaattggagatatcagacaat | |
| | caaattgttcgctcatgccatcaatcagttgatgggcctacctcacttcttcgaatggattcacct | |
| | aagactgatggacactacgatgttcgtaggagaccctttcaatcctccaagtgaccctactga | |
| | ctgtgacctctcaagagtccctaatgatgacatatatattgtcagtgccagagggggtatcga | |
| | aggattatgccagaagctatggacaatgatctcaattgctgcaatccaacttgctgcagctag | |
| | atcgcattgtcgtgttgcctgtatggtacagggtgataatcaagtaatagcagtaacgagaga | |
| | ggtaagatcagacgactctccggagatggtgttgacacagttgcatcaagccagtgataattt | |
| | cttcaaggaattaattcatgtcaatcatttgattggccataatttgaaggatcgtgaaaccatca | |
| | ggtcagacacattcttcatatacagcaaacgaatcttcaaagatggagcaatcctcagtcaag | |
| | tcctcaaaaattcatctaaattagtgctagtgtcaggtgatctcagtgaaaacaccgtaatgtcc | |
| | tgtgccaacattgcctctactgtagcacggctatgcgagaacgggcttcccaaagacttctgtt | |
| | actatttaaactatataatgagttgtgtgcagacatactttgactctgagttctccatcaccaaca | |
| | attcgcaccccgatcttaatcagtcgtggattgaggacatctcttttgtgcactcatatgttctga | |
| | ctcctgcccaattagggggactgagtaaccttcaatactcaaggctctacactagaaatatcg | |
| | gtgacccggggactactgcttttgcagagatcaagcgactagaagcagtgggattactgagt | |
| | cctaacattatgactaatatcttaactaggccgcctgggaatggagattgggccagtctgtgc | |
| | aacgacccatactctttcaattttgagactgttgcaagcccaaatattgttcttaagaaacatac | |
| | gcaaagagtcctatttgaaacttgttcaaatcccttattgtctggagtgcacacagaggataat | |
| | gaggcagaagagaaggcattggctgaattcttgcttaatcaagaggtgattcatccccgcgtt | |
| | gogcatgccatcatggaggcaagctctgtaggtaggagaaagcaaattcaagggcttgttg | |
| | acacaacaaacaccgtaattaagattgcgcttactaggaggccattaggcatcaagaggctg | |
| | atgcggatagtcaattattctagcatgcatgcaatgctgtttagagacgatgttttttcctccagt | |
| | agatccaaccaccccttagtctcttctaatatgtgttctctgacactggcagactatgcacgga | |
| | atagaagctggtcacctttgacgggaggcaggaaaatactgggtgtatctaatcctgatacg | |
| | atagaactcgtagagggtgagattcttagtgtaagcggagggtgtacaagatgtgacagcg | |
| | gagatgaacaatttacttggttccatcttccaagcaatatagaattgaccgatgacaccagcaa | |
| | gaatcctccgatgagggtaccatatctcgggtcaaagacacaggagaggagagctgcctca | |
| | cttgcaaaaatagctcatatgtcgccacatgtaaaggctgccctaagggcatcatccgtgttg | |
| | atctgggcttatggggataatgaagtaaattggactgctgctcttacgattgcaaaatctcggt | |
| | gtaatgtaaacttagagtatcttcggttactgtcccctttacccacggctgggaatcttcaacat | |
| | agactagatgatggtataactcagatgacattcacccctgcatctctctacagggtgtcacctt | |
| | acattcacatatccaatgattctcaaaggctgttcactgaagaaggagtcaaagaggggaat | |
| | gtggtttaccaacagatcatgctcttgggtttatctctaatcgaatcgatctttccaatgacaaca | |
| | accaggacatatgatgagatcacactgcacctacatagtaaatttagttgctgtatcagagaa | |
| | gcacctgttgcggttcctttcgagctacttggggggtaccggaactgaggacagtgacctca | |
| | aataagtttatgtatgatcctagccctgtatcggagggagactttgcgagacttgacttagctat | |
| | cttcaagagttatgagcttaatctggagtcatatcccacgatagagctaatgaacattctttcaat | |
| | atccagcgggaagttgattggccagtctgtggtttcttatgatgaagatacctccataaagaat | |
| | gacgccataatagtgtatgacaatacccgaaattggatcagtgaagctcagaattcagatgtg | |
| | gtccgcctatttgaatatgcagcacttgaagtgctcctcgactgttcttaccaactctattacctg | |
| | agagtaagaggcctggacaatattgtcttatatatgggtgatttatacaagaatatgccaggaa | |
| | ttctactttccaacattgcagctacaatatctcatcccgtcattcattcaaggttacatgcagtgg | |
| | gcctggtcaaccatgacggatcacaccaacttgcagatacggattttatcgaaatgtctgcaa | |
| | aactattagtatcttgcacccgacgtgtgatctccggcttatattcaggaaataagtatgatctg | |
| | ctgttcccatctgtcttagatgataacctgaatgagaagatgcttcagctgatatcccggttatg | |
| | ctgtctgtacacggtactctttgctacaacaagagaaatcccgaaaataagaggcttaactgc | |
| | agaagagaaatgttcaatactcactgagtatttactgtcggatgctgtgaaaccattacttagcc | |
| | ccgatcaagtgagctctatcatgtctcctaacataattacattcccagctaatctgtactacatgt | |
| | ctcggaagagcctcaatttgatcagggaaagggaggacagggatactatcctggcgttgttg | |
| | ttcccccaagagccattattagagttcccttctgtgcaagatattggtgctcgagtgaaagatc | |
| | cattcacccgacaacctgcggcatttttgcaagagttagatttgagtgctccagcaaggtatga | |
| | cgcattcacacttagtcagattcatcctgaactcacatctccaaatccggaggaagactactta | |
| | gtacgatacttgttcagagggatagggactgcatcttcctcttggtataaggcatctcatctcct | |
| | ttctgtacccgaggtaagatgtgcaagacacgggaactccttatacttagctgaagggagcg | |
| | gagccatcatgagtcttctcgaactgcatgtaccacatgaaactatctattacaatacgctctttt | |
| | caaatgagatgaaccccccgcaacgacatttcgggccgaccccaactcagtttttgaattcg | |
| | gttgtttataggaatctacaggcggaggtaacatgcaaagatggatttgtccaagagttccgtc | |
| | cattatggagagaaaatacagaggaaagcgacctgacctcagataaagtagtggggtatatt | |
| | acatctgcagtgccctacagatctgtatcattgctgcattgtgacattgaaattcctccagggtc | |
| | caatcaaagcttactagatcaactagctatcaatttatctctgattgccatgcattctgtaaggga | |
| | gggcggggtagtaatcatcaaagtgttgtatgcaatgggatactactttcatctactcatgaact | |
| | tgtttgctccgtgttccacaaaaggatatattctctctaatggttatgcatgtcgaggagatatgg | |
| | agtgttacctggtatttgtcatgggttacctgggcgggcctacatttgtacatgaggtggtgag | |
| | gatggcgaaaactctggtgcagcggcacggtacgcttttgtctaaatcagatgagatcacact | |
| | gaccaggttattcacctcacagcggcagcgtgtgacagacatcctatccagtcctttaccaag | |
| | attaataaagtacttgaggaagaatattgacactgcgctgattgaagccgggggacagcccg | |
| | tccgtccattctgtgcggagagtctggtgagcacgctagcgaacataactcagataacccag | |
| | atcatcgctagtcacattgacacagttatccggtctgtgatatatatggaagctgagggtgatct | |
| | cgctgacacagtatttctatttaccccttacaatctctctactgacgggaaaaagaggacatca | |
| | cttaaacagtgcacgagacagatcctagaggttacaatactaggtcttagagtcgaaaatctc | |
| | aataaaataggcgatataatcagcctagtgcttaaaggcatgatctccatggaggaccttatc | |
| | ccactaaggacatacttgaagcatagtacctgccctaaatatttgaaggctgtcctaggtatta | |
| | ccaaactcaaagaaatgtttacagacacttctgtactgtacttgactcgtgctcaacaaaaattc | |
| | tacatgaaaactataggcaatgcagtcaaaggatattacagtaactgtgactcttaacgaaaat | |
| | cacatattaataggctccttttttggccaattgtattcttgttgatttaatcatattatgttagaaa | |
| | aaagttgaaccctgactccttaggactcgaattcgaactcaaataaatgtcttaaaaaaaggttgc | |
| | gcacaattattcttgagtgtagtctcgtcattcaccaaatctttgtttggt | |

TABLE 2
NDV LaSota F protein

| | | SEQ ID |
|---|---|---|
| Description | Sequence | NO: |
| Amino acid | MGSRPSTKNPAPMTLTIRVALVLSCICPANSIDGRPLAAAG | SEQ ID |
| sequence of F | IVVTGDKAVNIYTSSQTGSIIVKLLPNLPKDKEACAKAPLD | NO: 4 |
| protein of NDV | AYNRTLTTLLTPLGDSIRRIQESVTTSGGGRQGRLIGAIIGG | |
| strain LaSota | VALGVATAAQITAAAALIQAKQNAANILRLKESIAATNEA | |
| (transmembrane | VHEVTDGLSQLAVAVGKMQQFVNDQFNKTAQELDCIKIA | |
| domain is | QQVGVELNLYLTELTTVFGPQITSPALNKLTIQALYNLAG | |
| underlined and | GNMDYLLTKLGVGNNQLSSLIGSGLITGNPILYDSQTQLLG | |
| cytoplasmic | IQVTLPSVGNLNNMRATYLETLSVSTTRGFASALVPKVVT | |
| domain is in | QVGSVIEELDTSYCIETDLDLYCTRIVTFPMSPGIYSCLSGN | |
| bold) | TSACMYSKTEGALTTPYMTIKGSVIANCKMTTCRCVNPPG | |
| | IISQNYGEAVSLIDKQSCNVLSLGGITLRLSGEFDVTYQKNI | |
| | SIQDSQVIITGNLDISTELGNVNNSISNALNKLEESNRKLDK | |
| | VNVKLTSTSALITYIVLTIISLVFGILSLILACYLMYKQKAQ | |
| | QKTLLWLGNNTLDQMRATTKM | |
| Amino acid | LITYIVLTIISLVFGILSLILACYLMYKQKAQQKTLLWLGNN | SEQ ID |
| sequence of | TLDQMRATTKM | NO:5 |
| transmembrane | | |
| and cytoplasmic | | |
| domains of F | | |
| protein of NDV | | |
| strain LaSota | | |

TABLE 3
SARS-CoV-2 Omicron Spike Nucleotide and Protein Sequences

Nucleotide sequence of Omicron BA.1: SEQ ID NO: 6 (signal peptide in bold; HXP-S
ectodomain; GS linker sequence in bold and underlined; transmembrane and
cytoplasmic domains of NDV F protein double underlined)
ATGTTCGTGTTTCTGGTGCTGCTGCCTCTGGTGTCCAGCCAGTGTGTGAACCT
GACCACAAGAACCCAGCTGCCTCCAGCCTACACCAACAGCTTTACCAGAGGCGT
GTACTACCCCGACAAGGTGTTCAGATCCAGCGTGCTGCACTCTACCCAGGACCTG
TTCCTGCCTTTCTTCAGCAACGTGACCTGGTTCCACgtgATCTCCGGCACCAATGGC
ACCAAGAGATTCGACAACCCCGTGCTGCCCTTCAACGACGGGGTGTACTTTGCCA
GCatcGAGAAGTCCAACATCATCAGAGGCTGGATCTTCGGCACCACACTGGACAG
CAAGACCCAGAGCCTGCTGATCGTGAACAACGCCACCAACGTGGTCATCAAAGT
GTGCGAGTTCCAGTTCTGCAACGACCCCTTCCTGgacCACAAGAACAACAAGAGC
TGGATGGAAAGCGAGTTCCGGGTGTACAGCAGCGCCAACAACTGCACCTTCGAG
TACGTGTCCCAGCCTTTCCTGATGGACCTGGAAGGCAAGCAGGGCAACTTCAAG
AACCTGCGCGAGTTCGTGTTCAAGAACATCGACGGCTACTTCAAGATCTACAGCA
AGCACACCCCTATCatcGTGCGGgaacccgaaGATCTGCCTCAGGGCTTCTCTGCTCTG
GAACCCCTGGTGGATCTGCCCATCGGCATCAACATCACCCGGTTTCAGACACTGC
TGGCCCTGCACAGAAGCTACCTGACACCTGGCGATAGCAGCAGCGGATGGACAG
CTGGTGCCGCCGCTTACTATGTGGGCTACCTGCAGCCTAGAACCTTTCTGCTGAA
GTACAACGAGAACGGCACCATCACCGACGCCGTGGATTGTGCTCTGGATCCTCTG
AGCGAGACAAAGTGCACCCTGAAGTCCTTCACCGTGGAgAAGGGCATCTACCAG
ACCAGCAACTTCCGGGTGCAGCCCACCGAATCCATCGTGCGGTTCCCCAATATCA
CCAATCTGTGCCCCTTCgacGAGGTGTTCAATGCCACCAGATTCGCCTCTGTGTAC
GCCTGGAACCGGAAGCGGATCAGCAATTGCGTGGCCGACTACTCCGTGCTGTAC
AACctgGCCcctTTCttcACCTTCAAGTGCTACGGCGTGTCCCCTACCAAGCTGAACGA
CCTGTGCTTCACAAACGTGTACGCCGACAGCTTCGTGATCCGGGGAGATGAAGT
GCGGCAGATTGCCCCTGGACAGACAGGCaacATCGCCGACTACAACTACAAGCTG
CCCGACGACTTCACCGGCTGTGTGATTGCCTGGAACAGCAACaagCTGGACTCCAA
AGTCagcGGCAACTACAATTACCTGTACCGGCTGTTCCGGAAGTCCAATCTGAAGC
CCTTCGAGCGGGACATCTCCACCGAGATCTATCAGGCCGGCaacaagCCTTGTAACG
GCGTGgccGGCTTCAACTGCTACTTCCCACTGagaTCCTACagcTTTagaCCCACAtacGG
CGTGGGCcacCAGCCCTACAGAGTGGTGGTGCTGAGCTTCGAACTGCTGCATGCCC
CTGCCACAGTGTGCGGCCCTAAGAAgAGCACCAATCTCGTGAAGAACAAATGCG
TGAACTTCAACTTCAACGGCCTGaagGGCACCGGCGTGCTGACAGAGAGCAACAA
GAAGTTCCTGCCATTCCAGCAGTTTGGCCGGGATATCGCCGATACCACAGACGCC
GTTAGAGATCCCCAGACACTGGAAATCCTGGACATCACCCCTTGCAGCTTCGGCG
GAGTGTCTGTGATCACCCCTGGCACCAACACCAGCAATCAGGTGGCAGTGCTGT
ACCAGggcGTGAACTGTACCGAAGTGCCCGTGGCCATTCACGCCGATCAGCTGAC
ACCTACATGGCGGGTGTACTCCACCGGCAGCAATGTGTTTCAGACCAGAGCCGG
CTGTCTGATCGGAGCCGAGtacGTGAACAATAGCTACGAGTGCGACATCCCCATCG
GCGCTGGCATCTGTGCCAGCTACCAGACACAGACAaagAGCcacGCCTCTGTGGCC
AGCCAGAGCATCATTGCCTACACAATGTCTCTGGGCGCCGAGAACAGCGTGGCC
TACTCCAACAACTCTATCGCTATCCCCACCAACTTCACCATCAGCGTGACCACAG
AGATCCTGCCTGTGTCCATGACCAAGACCAGCGTGGACTGCACCATGTACATCTG
CGGCGATTCCACCGAGTGCTCCAACCTGCTGCTGCAGTACGGCAGCTTCTGCACC
CAGCTGaagAGAGCCCTGACAGGGATCGCCGTGGAACAGGACAAGAACACCCAA
GAGGTGTTCGCCCAAGTGAAGCAGATCTACAAGACCCCTCCTATCAAGtacTTCGG
CGGCTTCAATTTCAGCCAGATTCTGCCCGATCCTAGCAAGCCCAGCAAGCGGAGC
cctATCGAGGACCTGCTGTTCAACAAAGTGACACTGGCCGACGCCGGCTTCATCAA
GCAGTATGGCGATTGTCTGGGCGACATTGCCGCCAGGGATCTGATTTGCGCCCAG
AAGTTTaagGGACTGACAGTGCTGCCTCCTCTGCTGACCGATGAGATGATCGCCCA
GTACACATCTGCCCTGCTGGCCGGCACAATCACAAGCGGCTGGACATTTGGAGCT
GGCcctGCTCTGCAGATCCCCTTTccaATGCAGATGGCCTACCGGTTCAACGGCATC
GGAGTGACCCAGAATGTGCTGTACGAGAACCAGAAGCTGATCGCCAACCAGTTC
AACAGCGCCATCGGCAAGATCCAGGACAGCCTGAGCAGCACAcccAGCGCCCTGG
GAAAGCTGCAGGACGTGGTCAACcacAATGCCCAGGCACTGAACACCCTGGTCAA
GCAGCTGTCCTCCaagTTCGGCGCCATCAGCTCTGTGCTGAACGATATCttcAGCAG
ACTGGACccccctGAAGCCGAGGTGCAGATCGACAGACTGATCACCGGAAGGCTGC
AGTCCCTGCAGACCTACGTTACCCAGCAGCTGATCAGAGCCGCCGAGATTAGAG
CCTCTGCCAATCTGGCCGCCACCAAGATGTCTGAGTGTGTGCTGGGCCAGAGCAA
GAGAGTGGACTTTTGCGGCAAGGGCTACCACCTGATGAGCTTCCCTCAGTCTGCC
CCTCACGGCGTGGTGTTTCTGCACGTGACATACGTGCCCGCTCAAGAGAAGAATT
TCACCACCGCTCCAGCCATCTGCCACGACGGCAAAGCCCACTTTCCTAGAGAAG
GCGTGTTCGTGTCCAACGGCACCCATTGGTTCGTGACCCAGCGGAACTTCTACGA
GCCCCAGATCATCACCACCGACAACACCTTCGTGTCTGGCAACTGCGACGTCGTG
ATCGGCATTGTGAACAATACCGTGTACGACCCTCTGCAGCCCGAGCTGGACAGCT
TCAAAGAGGAACTGGATAAGTACTTTAAGAACCACACAAGCCCCGACGTGGACC
TGGGCGATATCAGCGGAATCAATGCCAGCGTCGTGAACATCCAGAAAGAGATCG
ACCGGCTGAACGAGGTGGCCAAGAATCTGAACGAGAGCCTGATCGACCTGCAAG
AACTGGGGAAGTACGAGCAGTACATCAAGTGGCCCggcggaggtgggtcgCTCATAACA
TACATCGTCCTGACTATAATCAGCTTGGTATTTGGTATTTTGTCTTTGATTCTTGC
ATGCTATTTGATGTATAAACAGAAAGCTCAGCAGAAGACTCTCCTGTGGCTCGGT
AACAACACACTCGACCAGATGAGAGCAACTACAAAGATGTGA
Nucleotide sequence of Omicron BA.1: SEQ ID NO: 7 (HXP-S ectodomain; GS linker
sequence in bold and underlined; transmembrane and cytoplasmic domains of NDV F
protein double underlined)
CAGTGTGTGAACCTGACCACAAGAACCCAGCTGCCTCCAGCCTACACCAACAGC
TTTACCAGAGGCGTGTACTACCCCGACAAGGTGTTCAGATCCAGCGTGCTGCACT
CTACCCAGGACCTGTTCCTGCCTTTCTTCAGCAACGTGACCTGGTTCCACgtgATCT
CCGGCACCAATGGCACCAAGAGATTCGACAACCCCGTGCTGCCCTTCAACGACG
GGGTGTACTTTGCCAGCatcGAGAAGTCCAACATCATCAGAGGCTGGATCTTCGGC
ACCACACTGGACAGCAAGACCCAGAGCCTGCTGATCGTGAACAACGCCACCAAC
GTGGTCATCAAAGTGTGCGAGTTCCAGTTCTGCAACGACCCCTTCCTGgacCACAA
GAACAACAAGAGCTGGATGGAAAGCGAGTTCCGGGTGTACAGCAGCGCCAACA
ACTGCACCTTCGAGTACGTGTCCCAGCCTTTCCTGATGGACCTGGAAGGCAAGCA
GGGCAACTTCAAGAACCTGCGCGAGTTCGTGTTCAAGAACATCGACGGCTACTTC
AAGATCTACAGCAAGCACACCCCTATCatcGTGCGGgaacccgaaGATCTGCCTCAGGG
CTTCTCTGCTCTGGAACCCCTGGTGGATCTGCCCATCGGCATCAACATCACCCGG
TTTCAGACACTGCTGGCCCTGCACAGAAGCTACCTGACACCTGGCGATAGCAGC
AGCGGATGGACAGCTGGTGCCGCCGCTTACTATGTGGGCTACCTGCAGCCTAGA
ACCTTTCTGCTGAAGTACAACGAGAACGGCACCATCACCGACGCCGTGGATTGT
GCTCTGGATCCTCTGAGCGAGACAAAGTGCACCCTGAAGTCCTTCACCGTGGAgA
AGGGCATCTACCAGACCAGCAACTTCCGGGTGCAGCCCACCGAATCCATCGTGC
GGTTCCCCAATATCACCAATCTGTGCCCCTTCgacGAGGTGTTCAATGCCACCAGA
TTCGCCTCTGTGTACGCCTGGAACCGGAAGCGGATCAGCAATTGCGTGGCCGACT
ACTCCGTGCTGTACAACctgGCCcctTTCttcACCTTCAAGTGCTACGGCGTGTCCCCT
ACCAAGCTGAACGACCTGTGCTTCACAAACGTGTACGCCGACAGCTTCGTGATCC
GGGGAGATGAAGTGCGGCAGATTGCCCCTGGACAGACAGGCaacATCGCCGACTA
CAACTACAAGCTGCCCGACGACTTCACCGGCTGTGTGATTGCCTGGAACAGCAA
CaagCTGGACTCCAAAGTCagcGGCAACTACAATTACCTGTACCGGCTGTTCCGGAA
GTCCAATCTGAAGCCCTTCGAGCGGGACATCTCCACCGAGATCTATCAGGCCGGC
aacaagCCTTGTAACGGCGTGgccGGCTTCAACTGCTACTTCCCACTGagaTCCTACagc
TTTagaCCCACAtacGGCGTGGGCcacCAGCCCTACAGAGTGGTGGTGCTGAGCTTCG
AACTGCTGCATGCCCCTGCCACAGTGTGCGGCCCTAAGAAgAGCACCAATCTCGT
GAAGAACAAATGCGTGAACTTCAACTTCAACGGCCTGaagGGCACCGGCGTGCTG
ACAGAGAGCAACAAGAAGTTCCTGCCATTCCAGCAGTTTGGCCGGGATATCGCC
GATACCACAGACGCCGTTAGAGATCCCCAGACACTGGAAATCCTGGACATCACC
CCTTGCAGCTTCGGCGGAGTGTCTGTGATCACCCCTGGCACCAACACCAGCAATC
AGGTGGCAGTGCTGTACCAGggcGTGAACTGTACCGAAGTGCCCGTGGCCATTCA
CGCCGATCAGCTGACACCTACATGGCGGGTGTACTCCACCGGCAGCAATGTGTTT
CAGACCAGAGCCGGCTGTCTGATCGGAGCCGAGtacGTGAACAATAGCTACGAGT
GCGACATCCCCATCGGCGCTGGCATCTGTGCCAGCTACCAGACACAGACAaagAG
CcacGCCTCTGTGGCCAGCCAGAGCATCATTGCCTACACAATGTCTCTGGGCGCCG
AGAACAGCGTGGCCTACTCCAACAACTCTATCGCTATCCCCACCAACTTCACCAT
CAGCGTGACCACAGAGATCCTGCCTGTGTCCATGACCAAGACCAGCGTGGACTG
CACCATGTACATCTGCGGCGATTCCACCGAGTGCTCCAACCTGCTGCTGCAGTAC
GGCAGCTTCTGCACCCAGCTGaagAGAGCCCTGACAGGGATCGCCGTGGAACAGG
ACAAGAACACCCAAGAGGTGTTCGCCCAAGTGAAGCAGATCTACAAGACCCCTC
CTATCAAGtacTTCGGCGGCTTCAATTTCAGCCAGATTCTGCCCGATCCTAGCAAG
CCCAGCAAGCGGAGCcctATCGAGGACCTGCTGTTCAACAAAGTGACACTGGCCG
ACGCCGGCTTCATCAAGCAGTATGGCGATTGTCTGGGCGACATTGCCGCCAGGG
ATCTGATTTGCGCCCAGAAGTTTaagGGACTGACAGTGCTGCCTCCTCTGCTGACC
GATGAGATGATCGCCCAGTACACATCTGCCCTGCTGGCCGGCACAATCACAAGC
GGCTGGACATTTGGAGCTGGCcctGCTCTGCAGATCCCCTTTccaATGCAGATGGCC
TACCGGTTCAACGGCATCGGAGTGACCCAGAATGTGCTGTACGAGAACCAGAAG
CTGATCGCCAACCAGTTCAACAGCGCCATCGGCAAGATCCAGGACAGCCTGAGC
AGCACAcccAGCGCCCTGGGAAAGCTGCAGGACGTGGTCAACcacAATGCCCAGGC
ACTGAACACCCTGGTCAAGCAGCTGTCCTCCaagTTCGGCGCCATCAGCTCTGTGC
TGAACGATATCttcAGCAGACTGGACccccctGAAGCCGAGGTGCAGATCGACAGACT
GATCACCGGAAGGCTGCAGTCCCTGCAGACCTACGTTACCCAGCAGCTGATCAG
AGCCGCCGAGATTAGAGCCTCTGCCAATCTGGCCGCCACCAAGATGTCTGAGTGT
GTGCTGGGCCAGAGCAAGAGAGTGGACTTTTGCGGCAAGGGCTACCACCTGATG
AGCTTCCCTCAGTCTGCCCCTCACGGCGTGGTGTTTCTGCACGTGACATACGTGC
CCGCTCAAGAGAAGAATTTCACCACCGCTCCAGCCATCTGCCACGACGGCAAAG
CCCACTTTCCTAGAGAAGGCGTGTTCGTGTCCAACGGCACCCATTGGTTCGTGAC
CCAGCGGAACTTCTACGAGCCCCAGATCATCACCACCGACAACACCTTCGTGTCT
GGCAACTGCGACGTCGTGATCGGCATTGTGAACAATACCGTGTACGACCCTCTGC
AGCCCGAGCTGGACAGCTTCAAAGAGGAACTGGATAAGTACTTTAAGAACCACA
CAAGCCCCGACGTGGACCTGGGCGATATCAGCGGAATCAATGCCAGCGTCGTGA
ACATCCAGAAAGAGATCGACCGGCTGAACGAGGTGGCCAAGAATCTGAACGAG
AGCCTGATCGACCTGCAAGAACTGGGGAAGTACGAGCAGTACATCAAGTGGCCC
ggcggaggtgggtcgCTCATAACATACATCGTCCTGACTATAATCAGCTTGGTATTTGGT
ATTTTGTCTTTGATTCTTGCATGCTATTTGATGTATAAACAGAAAGCTCAGCAGA
AGACTCTCCTGTGGCTCGGTAACAACACACTCGACCAGATGAGAGCAACTACAA
AGATGTGA
Amino acid sequence of Omicron BA.1: SEQ ID NO: 8 (signal peptide in bold; HXP-S
ectodomain; GS linker sequence in bold and underlined; transmembrane and
cytoplasmic domains of NDV F protein double underlined)
MFVFLVLLPLVSSQCVNLTTRTQLPPAYTNSFTRGVYYPDKVFRSSVLHSTQDLFLP
FFSNVTWFHVISGTNGTKRFDNPVLPFNDGVYFASIEKSNIIRGWIFGTTLDSKTQSLL
IVNNATNVVIKVCEFQFCNDPFLDHKNNKSWMESEFRVYSSANNCTFEYVSQPFLM
DLEGKQGNFKNLREFVFKNIDGYFKIYSKHTPIIVREPEDLPQGFSALEPLVDLPIGINI
TRFQTLLALHRSYLTPGDSSSGWTAGAAAYYVGYLQPRTFLLKYNENGTITDAVDC
ALDPLSETKCTLKSFTVEKGIYQTSNFRVQPTESIVRFPNITNLCPFDEVFNATRFASV
YAWNRKRISNCVADYSVLYNLAPFFTFKCYGVSPTKLNDLCFTNVYADSFVIRGDEV
RQIAPGQTGNIADYNYKLPDDFTGCVIAWNSNKLDSKVSGNYNYLYRLFRKSNLKPF
ERDISTEIYQAGNKPCNGVAGFNCYFPLRSYSFRPTYGVGHQPYRVVVLSFELLHAP
ATVCGPKKSTNLVKNKCVNFNFNGLKGTGVLTESNKKFLPFQQFGRDIADTTDAVR
DPQTLEILDITPCSFGGVSVITPGTNTSNQVAVLYQGVNCTEVPVAIHADQLTPTWRV
YSTGSNVFQTRAGCLIGAEYVNNSYECDIPIGAGICASYQTQTKSHASVASQSIIAYT
MSLGAENSVAYSNNSIAIPTNFTISVTTEILPVSMTKTSVDCTMYICGDSTECSNLLLQ
YGSFCTQLKRALTGIAVEQDKNTQEVFAQVKQIYKTPPIKYFGGFNFSQILPDPSKPS
KRSPIEDLLFNKVTLADAGFIKQYGDCLGDIAARDLICAQKFKGLTVLPPLLTDEMIA
QYTSALLAGTITSGWTFGAGPALQIPFPMQMAYRENGIGVTQNVLYENQKLIANQFN
SAIGKIQDSLSSTPSALGKLQDVVNHNAQALNTLVKQLSSKFGAISSVLNDIFSRLDPP
EAEVQIDRLITGRLQSLQTYVTQQLIRAAEIRASANLAATKMSECVLGQSKRVDFCG
KGYHLMSFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKEELDKYFKN
HTSPDVDLGDISGINASVVNIQKEIDRLNEVAKNLNESLIDLQELGKYEQYIKWPGGG
GSLITYIVLTIISLVEGILSLILACYLMYKQKAQQKTLLWLGNNTLDQMRATTKM*
Amino acid sequence of Omicron BA.1: SEQ ID NO: 9 (HXP-S ectodomain; GS linker
sequence in bold and underlined; transmembrane and cytoplasmic domains of NDV F
protein double underlined)
QCVNLTTRTQLPPAYTNSFTRGVYYPDKVFRSSVLHSTQDLFLPFFSNVTWFHVISGT
NGTKRFDNPVLPFNDGVYFASIEKSNIIRGWIFGTTLDSKTQSLLIVNNATNVVIKVCE
FQFCNDPFLDHKNNKSWMESEFRVYSSANNCTFEYVSQPFLMDLEGKQGNFKNLRE
FVFKNIDGYFKIYSKHTPIIVREPEDLPQGFSALEPLVDLPIGINITRFQTLLALHRSYLT
PGDSSSGWTAGAAAYYVGYLQPRTFLLKYNENGTITDAVDCALDPLSETKCTLKSFT
VEKGIYQTSNFRVQPTESIVRFPNITNLCPFDEVFNATRFASVYAWNRKRISNCVADY
SVLYNLAPFFTFKCYGVSPTKLNDLCFTNVYADSFVIRGDEVRQIAPGQTGNIADYN
YKLPDDFTGCVIAWNSNKLDSKVSGNYNYLYRLFRKSNLKPFERDISTEIYQAGNKP
CNGVAGFNCYFPLRSYSFRPTYGVGHQPYRVVVLSFELLHAPATVCGPKKSTNLVK
NKCVNFNFNGLKGTGVLTESNKKFLPFQQFGRDIADTTDAVRDPQTLEILDITPCSFG
GVSVITPGTNTSNQVAVLYQGVNCTEVPVAIHADQLTPTWRVYSTGSNVFQTRAGC
LIGAEYVNNSYECDIPIGAGICASYQTQTKSHASVASQSIIAYTMSLGAENSVAYSNN
SIAIPTNFTISVTTEILPVSMTKTSVDCTMYICGDSTECSNLLLQYGSFCTQLKRALTGI
AVEQDKNTQEVFAQVKQIYKTPPIKYFGGFNFSQILPDPSKPSKRSPIEDLLFNKVTLA
DAGFIKQYGDCLGDIAARDLICAQKFKGLTVLPPLLTDEMIAQYTSALLAGTITSGWT
FGAGPALQIPFPMQMAYRENGIGVTQNVLYENQKLIANQFNSAIGKIQDSLSSTPSAL
GKLQDVVNHNAQALNTLVKQLSSKFGAISSVLNDIFSRLDPPEAEVQIDRLITGRLQS
LQTYVTQQLIRAAEIRASANLAATKMSECVLGQSKRVDFCGKGYHLMSFPQSAPHG
VVFLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISGINA
SVVNIQKEIDRLNEVAKNLNESLIDLQELGKYEQYIKWPGGGGSLITYIVLTIISLVFGI
LSLILACYLMYKQKAQQKTLLWLGNNTLDQMRATTKM*
Nucleotide sequence of Omicron BA.1 (H655_deCSV6871): SEQ ID NO: 10 (signal
peptide in bold; HXP-S ectodomain; GS linker sequence bold and underlined;
transmembrane and cytoplasmic domains of NDV F protein double underlined)
ATGTTCGTGTTTCTGGTGCTGCTGCCTCTGGTGTCCAGCCAGTGTGTGAACCT
GACCACAAGAACCCAGCTGCCTCCAGCCTACACCAACAGCTTTACCAGAGGCGT
GTACTACCCCGACAAGGTGTTCAGATCCAGCGTGCTGCACTCTACCCAGGACCTG
TTCCTGCCTTTCTTCAGCAACGTGACCTGGTTCCACgtgATCTCCGGCACCAATGGC
ACCAAGAGATTCGACAACCCCGTGCTGCCCTTCAACGACGGGGTGTACTTTGCCA
GCatcGAGAAGTCCAACATCATCAGAGGCTGGATCTTCGGCACCACACTGGACAG
CAAGACCCAGAGCCTGCTGATCGTGAACAACGCCACCAACGTGGTCATCAAAGT
GTGCGAGTTCCAGTTCTGCAACGACCCCTTCCTGgacCACAAGAACAACAAGAGC
TGGATGGAAAGCGAGTTCCGGGTGTACAGCAGCGCCAACAACTGCACCTTCGAG
TACGTGTCCCAGCCTTTCCTGATGGACCTGGAAGGCAAGCAGGGCAACTTCAAG
AACCTGCGCGAGTTCGTGTTCAAGAACATCGACGGCTACTTCAAGATCTACAGCA
AGCACACCCCTATCatcGTGCGGgaacccgaaGATCTGCCTCAGGGCTTCTCTGCTCTG
GAACCCCTGGTGGATCTGCCCATCGGCATCAACATCACCCGGTTTCAGACACTGC
TGGCCCTGCACAGAAGCTACCTGACACCTGGCGATAGCAGCAGCGGATGGACAG
CTGGTGCCGCCGCTTACTATGTGGGCTACCTGCAGCCTAGAACCTTTCTGCTGAA
GTACAACGAGAACGGCACCATCACCGACGCCGTGGATTGTGCTCTGGATCCTCTG
AGCGAGACAAAGTGCACCCTGAAGTCCTTCACCGTGGAgAAGGGCATCTACCAG
ACCAGCAACTTCCGGGTGCAGCCCACCGAATCCATCGTGCGGTTCCCCAATATCA
CCAATCTGTGCCCCTTCgacGAGGTGTTCAATGCCACCAGATTCGCCTCTGTGTAC
GCCTGGAACCGGAAGCGGATCAGCAATTGCGTGGCCGACTACTCCGTGCTGTAC
AACctgGCCcctTTCttcACCTTCAAGTGCTACGGCGTGTCCCCTACCAAGCTGAACGA
CCTGTGCTTCACAAACGTGTACGCCGACAGCTTCGTGATCCGGGGAGATGAAGT
GCGGCAGATTGCCCCTGGACAGACAGGCaacATCGCCGACTACAACTACAAGCTG
CCCGACGACTTCACCGGCTGTGTGATTGCCTGGAACAGCAACaagCTGGACTCCAA
AGTCagcGGCAACTACAATTACCTGTACCGGCTGTTCCGGAAGTCCAATCTGAAGC
CCTTCGAGCGGGACATCTCCACCGAGATCTATCAGGCCGGCaacaagCCTTGTAACG
GCGTGgccGGCTTCAACTGCTACTTCCCACTGagaTCCTACagcTTTagaCCCACAtacGG
CGTGGGCcacCAGCCCTACAGAGTGGTGGTGCTGAGCTTCGAACTGCTGCATGCCC
CTGCCACAGTGTGCGGCCCTAAGAAgAGCACCAATCTCGTGAAGAACAAATGCG
TGAACTTCAACTTCAACGGCCTGaagGGCACCGGCGTGCTGACAGAGAGCAACAA
GAAGTTCCTGCCATTCCAGCAGTTTGGCCGGGATATCGCCGATACCACAGACGCC
GTTAGAGATCCCCAGACACTGGAAATCCTGGACATCACCCCTTGCAGCTTCGGCG
GAGTGTCTGTGATCACCCCTGGCACCAACACCAGCAATCAGGTGGCAGTGCTGT
ACCAGggcGTGAACTGTACCGAAGTGCCCGTGGCCATTCACGCCGATCAGCTGAC
ACCTACATGGCGGGTGTACTCCACCGGCAGCAATGTGTTTCAGACCAGAGCCGG
CTGTCTGATCGGAGCCGAGcacGTGAACAATAGCTACGAGTGCGACATCCCCATC
GGCGCTGGCATCTGTGCCAGCTACCAGACACAGACAatcGCCAGCCAGAGCATCA
TTGCCTACACAATGTCTCTGGGCGCCGAGAACAGCGTGGCCTACTCCAACAACTC
TATCGCTATCCCCACCAACTTCACCATCAGCGTGACCACAGAGATCCTGCCTGTG
TCCATGACCAAGACCAGCGTGGACTGCACCATGTACATCTGCGGCGATTCCACCG
AGTGCTCCAACCTGCTGCTGCAGTACGGCAGCTTCTGCACCCAGCTGaagAGAGCC
CTGACAGGGATCGCCGTGGAACAGGACAAGAACACCCAAGAGGTGTTCGCCCAA
GTGAAGCAGATCTACAAGACCCCTCCTATCAAGtacTTCGGCGGCTTCAATTTCAG
CCAGATTCTGCCCGATCCTAGCAAGCCCAGCAAGCGGAGCcctATCGAGGACCTG
CTGTTCAACAAAGTGACACTGGCCGACGCCGGCTTCATCAAGCAGTATGGCGATT
GTCTGGGCGACATTGCCGCCAGGGATCTGATTTGCGCCCAGAAGTTTaagGGACTG
ACAGTGCTGCCTCCTCTGCTGACCGATGAGATGATCGCCCAGTACACATCTGCCC
TGCTGGCCGGCACAATCACAAGCGGCTGGACATTTGGAGCTGGCcctGCTCTGCAG
ATCCCCTTTccaATGCAGATGGCCTACCGGTTCAACGGCATCGGAGTGACCCAGAA
TGTGCTGTACGAGAACCAGAAGCTGATCGCCAACCAGTTCAACAGCGCCATCGG
CAAGATCCAGGACAGCCTGAGCAGCACAcccAGCGCCCTGGGAAAGCTGCAGGAC
GTGGTCAACcacAATGCCCAGGCACTGAACACCCTGGTCAAGCAGCTGTCCTCCaag
TTCGGCGCCATCAGCTCTGTGCTGAACGATATCttcAGCAGACTGGACccccctGAAG
CCGAGGTGCAGATCGACAGACTGATCACCGGAAGGCTGCAGTCCCTGCAGACCT
ACGTTACCCAGCAGCTGATCAGAGCCGCCGAGATTAGAGCCTCTGCCAATCTGG
CCGCCACCAAGATGTCTGAGTGTGTGCTGGGCCAGAGCAAGAGAGTGGACTTTT
GCGGCAAGGGCTACCACCTGATGAGCTTCCCTCAGTCTGCCCCTCACGGCGTGGT
GTTTCTGCACGTGACATACGTGCCCGCTCAAGAGAAGAATTTCACCACCGCTCCA
GCCATCTGCCACGACGGCAAAGCCCACTTTCCTAGAGAAGGCGTGTTCGTGTCCA
ACGGCACCCATTGGTTCGTGACCCAGCGGAACTTCTACGAGCCCCAGATCATCAC
CACCGACAACACCTTCGTGTCTGGCAACTGCGACGTCGTGATCGGCATTGTGAAC
AATACCGTGTACGACCCTCTGCAGCCCGAGCTGGACAGCTTCAAAGAGGAACTG
GATAAGTACTTTAAGAACCACACAAGCCCCGACGTGGACCTGGGCGATATCAGC
GGAATCAATGCCAGCGTCGTGAACATCCAGAAAGAGATCGACCGGCTGAACGAG
GTGGCCAAGAATCTGAACGAGAGCCTGATCGACCTGCAAGAACTGGGGAAGTAC
GAGCAGTACATCAAGTGGCCCggcggaggtgggtcgCTCATAACATACATCGTCCTGAC
TATAATCAGCTTGGTATTTGGTATTTTGTCTTTGATTCTTGCATGCTATTTGATGTA
TAAACAGAAAGCTCAGCAGAAGACTCTCCTGTGGCTCGGTAACAACACACTCGA
CCAGATGAGAGCAACTACAAAGATGTGA
Nucleotide sequence of Omicron BA.1 (H655_deCSV687I): SEQ ID NO: 11 (HXP-S
ectodomain; GS linker sequence bold and underlined; transmembrane and cytoplasmic
domains of NDV F protein double underlined)
CAGTGTGTGAACCTGACCACAAGAACCCAGCTGCCTCCAGCCTACACCAACAGC
TTTACCAGAGGCGTGTACTACCCCGACAAGGTGTTCAGATCCAGCGTGCTGCACT
CTACCCAGGACCTGTTCCTGCCTTTCTTCAGCAACGTGACCTGGTTCCACgtgATCT
CCGGCACCAATGGCACCAAGAGATTCGACAACCCCGTGCTGCCCTTCAACGACG
GGGTGTACTTTGCCAGCatcGAGAAGTCCAACATCATCAGAGGCTGGATCTTCGGC
ACCACACTGGACAGCAAGACCCAGAGCCTGCTGATCGTGAACAACGCCACCAAC
GTGGTCATCAAAGTGTGCGAGTTCCAGTTCTGCAACGACCCCTTCCTGgacCACAA
GAACAACAAGAGCTGGATGGAAAGCGAGTTCCGGGTGTACAGCAGCGCCAACA
ACTGCACCTTCGAGTACGTGTCCCAGCCTTTCCTGATGGACCTGGAAGGCAAGCA
GGGCAACTTCAAGAACCTGCGCGAGTTCGTGTTCAAGAACATCGACGGCTACTTC
AAGATCTACAGCAAGCACACCCCTATCatcGTGCGGgaacccgaaGATCTGCCTCAGGG
CTTCTCTGCTCTGGAACCCCTGGTGGATCTGCCCATCGGCATCAACATCACCCGG
TTTCAGACACTGCTGGCCCTGCACAGAAGCTACCTGACACCTGGCGATAGCAGC
AGCGGATGGACAGCTGGTGCCGCCGCTTACTATGTGGGCTACCTGCAGCCTAGA
ACCTTTCTGCTGAAGTACAACGAGAACGGCACCATCACCGACGCCGTGGATTGT
GCTCTGGATCCTCTGAGCGAGACAAAGTGCACCCTGAAGTCCTTCACCGTGGAgA
AGGGCATCTACCAGACCAGCAACTTCCGGGTGCAGCCCACCGAATCCATCGTGC
GGTTCCCCAATATCACCAATCTGTGCCCCTTCgacGAGGTGTTCAATGCCACCAGA
TTCGCCTCTGTGTACGCCTGGAACCGGAAGCGGATCAGCAATTGCGTGGCCGACT
ACTCCGTGCTGTACAACctgGCCcctTTCttcACCTTCAAGTGCTACGGCGTGTCCCCT
ACCAAGCTGAACGACCTGTGCTTCACAAACGTGTACGCCGACAGCTTCGTGATCC
GGGGAGATGAAGTGCGGCAGATTGCCCCTGGACAGACAGGCaacATCGCCGACTA
CAACTACAAGCTGCCCGACGACTTCACCGGCTGTGTGATTGCCTGGAACAGCAA
CaagCTGGACTCCAAAGTCagcGGCAACTACAATTACCTGTACCGGCTGTTCCGGAA
GTCCAATCTGAAGCCCTTCGAGCGGGACATCTCCACCGAGATCTATCAGGCCGGC
aacaagCCTTGTAACGGCGTGgccGGCTTCAACTGCTACTTCCCACTGagaTCCTACagc
TTTagaCCCACAtacGGCGTGGGCcacCAGCCCTACAGAGTGGTGGTGCTGAGCTTCG
AACTGCTGCATGCCCCTGCCACAGTGTGCGGCCCTAAGAAgAGCACCAATCTCGT
GAAGAACAAATGCGTGAACTTCAACTTCAACGGCCTGaagGGCACCGGCGTGCTG
ACAGAGAGCAACAAGAAGTTCCTGCCATTCCAGCAGTTTGGCCGGGATATCGCC
GATACCACAGACGCCGTTAGAGATCCCCAGACACTGGAAATCCTGGACATCACC
CCTTGCAGCTTCGGCGGAGTGTCTGTGATCACCCCTGGCACCAACACCAGCAATC
AGGTGGCAGTGCTGTACCAGggcGTGAACTGTACCGAAGTGCCCGTGGCCATTCA
CGCCGATCAGCTGACACCTACATGGCGGGTGTACTCCACCGGCAGCAATGTGTTT
CAGACCAGAGCCGGCTGTCTGATCGGAGCCGAGcacGTGAACAATAGCTACGAGT
GCGACATCCCCATCGGCGCTGGCATCTGTGCCAGCTACCAGACACAGACAatcGCC
AGCCAGAGCATCATTGCCTACACAATGTCTCTGGGCGCCGAGAACAGCGTGGCC
TACTCCAACAACTCTATCGCTATCCCCACCAACTTCACCATCAGCGTGACCACAG
AGATCCTGCCTGTGTCCATGACCAAGACCAGCGTGGACTGCACCATGTACATCTG
CGGCGATTCCACCGAGTGCTCCAACCTGCTGCTGCAGTACGGCAGCTTCTGCACC
CAGCTGaagAGAGCCCTGACAGGGATCGCCGTGGAACAGGACAAGAACACCCAA
GAGGTGTTCGCCCAAGTGAAGCAGATCTACAAGACCCCTCCTATCAAGtacTTCGG
CGGCTTCAATTTCAGCCAGATTCTGCCCGATCCTAGCAAGCCCAGCAAGCGGAGC
cctATCGAGGACCTGCTGTTCAACAAAGTGACACTGGCCGACGCCGGCTTCATCAA
GCAGTATGGCGATTGTCTGGGCGACATTGCCGCCAGGGATCTGATTTGCGCCCAG
AAGTTTaagGGACTGACAGTGCTGCCTCCTCTGCTGACCGATGAGATGATCGCCCA
GTACACATCTGCCCTGCTGGCCGGCACAATCACAAGCGGCTGGACATTTGGAGCT
GGCcctGCTCTGCAGATCCCCTTTccaATGCAGATGGCCTACCGGTTCAACGGCATC
GGAGTGACCCAGAATGTGCTGTACGAGAACCAGAAGCTGATCGCCAACCAGTTC
AACAGCGCCATCGGCAAGATCCAGGACAGCCTGAGCAGCACAcccAGCGCCCTGG
GAAAGCTGCAGGACGTGGTCAACcacAATGCCCAGGCACTGAACACCCTGGTCAA
GCAGCTGTCCTCCaagTTCGGCGCCATCAGCTCTGTGCTGAACGATATCttcAGCAG
ACTGGACccccctGAAGCCGAGGTGCAGATCGACAGACTGATCACCGGAAGGCTGC
AGTCCCTGCAGACCTACGTTACCCAGCAGCTGATCAGAGCCGCCGAGATTAGAG
CCTCTGCCAATCTGGCCGCCACCAAGATGTCTGAGTGTGTGCTGGGCCAGAGCAA
GAGAGTGGACTTTTGCGGCAAGGGCTACCACCTGATGAGCTTCCCTCAGTCTGCC
CCTCACGGCGTGGTGTTTCTGCACGTGACATACGTGCCCGCTCAAGAGAAGAATT
TCACCACCGCTCCAGCCATCTGCCACGACGGCAAAGCCCACTTTCCTAGAGAAG
GCGTGTTCGTGTCCAACGGCACCCATTGGTTCGTGACCCAGCGGAACTTCTACGA
GCCCCAGATCATCACCACCGACAACACCTTCGTGTCTGGCAACTGCGACGTCGTG
ATCGGCATTGTGAACAATACCGTGTACGACCCTCTGCAGCCCGAGCTGGACAGCT
TCAAAGAGGAACTGGATAAGTACTTTAAGAACCACACAAGCCCCGACGTGGACC
TGGGCGATATCAGCGGAATCAATGCCAGCGTCGTGAACATCCAGAAAGAGATCG
ACCGGCTGAACGAGGTGGCCAAGAATCTGAACGAGAGCCTGATCGACCTGCAAG
AACTGGGGAAGTACGAGCAGTACATCAAGTGGCCCggcggaggtgggtcgCTCATAACA
TACATCGTCCTGACTATAATCAGCTTGGTATTTGGTATTTTGTCTTTGATTCTTGC
ATGCTATTTGATGTATAAACAGAAAGCTCAGCAGAAGACTCTCCTGTGGCTCGGT
AACAACACACTCGACCAGATGAGAGCAACTACAAAGATGTGA
Amino acid sequence of Omicron BA.1 (H655_deCSV6871): SEQ ID NO: 12 (signal
peptide in bold; HXP-S ectodomain; GS linker sequence bold and underlined;
transmembrane and cytoplasmic domains of NDV F protein double underlined)
MFVFLVLLPLVSSQCVNLTTRTQLPPAYTNSFTRGVYYPDKVFRSSVLHSTQDLFLP
FFSNVTWFHVISGTNGTKRFDNPVLPFNDGVYFASIEKSNIIRGWIFGTTLDSKTQSLL
IVNNATNVVIKVCEFQFCNDPFLDHKNNKSWMESEFRVYSSANNCTFEYVSQPFLM
DLEGKQGNFKNLREFVFKNIDGYFKIYSKHTPIIVREPEDLPQGFSALEPLVDLPIGINI
TRFQTLLALHRSYLTPGDSSSGWTAGAAAYYVGYLQPRTFLLKYNENGTITDAVDC
ALDPLSETKCTLKSFTVEKGIYQTSNFRVQPTESIVRFPNITNLCPFDEVFNATRFASV
YAWNRKRISNCVADYSVLYNLAPFFTFKCYGVSPTKLNDLCFTNVYADSFVIRGDEV
RQIAPGQTGNIADYNYKLPDDFTGCVIAWNSNKLDSKVSGNYNYLYRLFRKSNLKPF
ERDISTEIYQAGNKPCNGVAGFNCYFPLRSYSFRPTYGVGHQPYRVVVLSFELLHAP
ATVCGPKKSTNLVKNKCVNFNFNGLKGTGVLTESNKKFLPFQQFGRDIADTTDAVR
DPQTLEILDITPCSFGGVSVITPGTNTSNQVAVLYQGVNCTEVPVAIHADQLTPTWRV
YSTGSNVFQTRAGCLIGAEHVNNSYECDIPIGAGICASYQTQTIASQSIIAYTMSLGAE
NSVAYSNNSIAIPTNFTISVTTEILPVSMTKTSVDCTMYICGDSTECSNLLLQYGSFCT
QLKRALTGIAVEQDKNTQEVFAQVKQIYKTPPIKYFGGFNFSQILPDPSKPSKRSPIED
LLFNKVTLADAGFIKQYGDCLGDIAARDLICAQKFKGLTVLPPLLTDEMIAQYTSALL
AGTITSGWTFGAGPALQIPFPMQMAYRFNGIGVTQNVLYENQKLIANQFNSAIGKIQ
DSLSSTPSALGKLQDVVNHNAQALNTLVKQLSSKFGAISSVLNDIFSRLDPPEAEVQI
DRLITGRLQSLQTYVTQQLIRAAEIRASANLAATKMSECVLGQSKRVDFCGKGYHL
MSFPQSAPHGVVFLHVTYVPAQEKNFTTAPAICHDGKAHFPREGVFVSNGTHWFVT
QRNFYEPQIITTDNTFVSGNCDVVIGIVNNTVYDPLQPELDSFKEELDKYFKNHTSPD
VDLGDISGINASVVNIQKEIDRLNEVAKNLNESLIDLQELGKYEQYIKWPGGGGSLIT
YIVLTIISLVFGILSLILACYLMYKQKAQQKTLLWLGNNTLDQMRATTKM*
Amino acid sequence of Omicron BA.1 (H655_deCSV6871): SEQ ID NO: 13 (HXP-S
ectodomain; GS linker sequence bold and underlined; transmembrane and cytoplasmic
domains of NDV F protein double underlined)
QCVNLTTRTQLPPAYTNSFTRGVYYPDKVFRSSVLHSTQDLFLPFFSNVTWFHVISGT
NGTKRFDNPVLPFNDGVYFASIEKSNIIRGWIFGTTLDSKTQSLLIVNNATNVVIKVCE
FQFCNDPFLDHKNNKSWMESEFRVYSSANNCTFEYVSQPFLMDLEGKQGNFKNLRE
FVFKNIDGYFKIYSKHTPIIVREPEDLPQGFSALEPLVDLPIGINITRFQTLLALHRSYLT
PGDSSSGWTAGAAAYYVGYLQPRTFLLKYNENGTITDAVDCALDPLSETKCTLKSFT
VEKGIYQTSNFRVQPTESIVRFPNITNLCPFDEVFNATRFASVYAWNRKRISNCVADY
SVLYNLAPFFTFKCYGVSPTKLNDLCFTNVYADSFVIRGDEVRQIAPGQTGNIADYN
YKLPDDFTGCVIAWNSNKLDSKVSGNYNYLYRLFRKSNLKPFERDISTEIYQAGNKP
CNGVAGFNCYFPLRSYSFRPTYGVGHQPYRVVVLSFELLHAPATVCGPKKSTNLVK
NKCVNFNFNGLKGTGVLTESNKKFLPFQQFGRDIADTTDAVRDPQTLEILDITPCSFG
GVSVITPGTNTSNQVAVLYQGVNCTEVPVAIHADQLTPTWRVYSTGSNVFQTRAGC
LIGAEHVNNSYECDIPIGAGICASYQTQTIASQSIIAYTMSLGAENSVAYSNNSIAIPTN
FTISVTTEILPVSMTKTSVDCTMYICGDSTECSNLLLQYGSFCTQLKRALTGIAVEQD
KNTQEVFAQVKQIYKTPPIKYFGGFNFSQILPDPSKPSKRSPIEDLLFNKVTLADAGFI
KQYGDCLGDIAARDLICAQKFKGLTVLPPLLTDEMIAQYTSALLAGTITSGWTFGAG
PALQIPFPMQMAYRFNGIGVTQNVLYENQKLIANQFNSAIGKIQDSLSSTPSALGKLQ
DVVNHNAQALNTLVKQLSSKFGAISSVLNDIFSRLDPPEAEVQIDRLITGRLQSLQTY
VTQQLIRAAEIRASANLAATKMSECVLGQSKRVDFCGKGYHLMSFPQSAPHGVVFL
HVTYVPAQEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASVVN
IQKEIDRLNEVAKNLNESLIDLQELGKYEQYIKWPGGGGSLITYIVLTIISLVFGILSLIL
ACYLMYKQKAQQKTLLWLGNNTLDQMRATTKM*
Nucleotide sequence of Omicron BA.1 (Q493Q498): SEQ ID NO: 14 (signal peptide in
bold; HXP-S ectodomain; GS linker sequence bold and underlined; transmembrane
and cytoplasmic domains of NDV F protein double underlined)
ATGTTCGTGTTTCTGGTGCTGCTGCCTCTGGTGTCCAGCCAGTGTGTGAACCT
GACCACAAGAACCCAGCTGCCTCCAGCCTACACCAACAGCTTTACCAGAGGCGT
GTACTACCCCGACAAGGTGTTCAGATCCAGCGTGCTGCACTCTACCCAGGACCTG
TTCCTGCCTTTCTTCAGCAACGTGACCTGGTTCCACgtgATCTCCGGCACCAATGGC
ACCAAGAGATTCGACAACCCCGTGCTGCCCTTCAACGACGGGGTGTACTTTGCCA
GCatcGAGAAGTCCAACATCATCAGAGGCTGGATCTTCGGCACCACACTGGACAG
CAAGACCCAGAGCCTGCTGATCGTGAACAACGCCACCAACGTGGTCATCAAAGT
GTGCGAGTTCCAGTTCTGCAACGACCCCTTCCTGgacCACAAGAACAACAAGAGC
TGGATGGAAAGCGAGTTCCGGGTGTACAGCAGCGCCAACAACTGCACCTTCGAG
TACGTGTCCCAGCCTTTCCTGATGGACCTGGAAGGCAAGCAGGGCAACTTCAAG
AACCTGCGCGAGTTCGTGTTCAAGAACATCGACGGCTACTTCAAGATCTACAGCA
AGCACACCCCTATCatcGTGCGGgaacccgaaGATCTGCCTCAGGGCTTCTCTGCTCTG
GAACCCCTGGTGGATCTGCCCATCGGCATCAACATCACCCGGTTTCAGACACTGC
TGGCCCTGCACAGAAGCTACCTGACACCTGGCGATAGCAGCAGCGGATGGACAG
CTGGTGCCGCCGCTTACTATGTGGGCTACCTGCAGCCTAGAACCTTTCTGCTGAA
GTACAACGAGAACGGCACCATCACCGACGCCGTGGATTGTGCTCTGGATCCTCTG
AGCGAGACAAAGTGCACCCTGAAGTCCTTCACCGTGGAgAAGGGCATCTACCAG
ACCAGCAACTTCCGGGTGCAGCCCACCGAATCCATCGTGCGGTTCCCCAATATCA
CCAATCTGTGCCCCTTCgacGAGGTGTTCAATGCCACCAGATTCGCCTCTGTGTAC
GCCTGGAACCGGAAGCGGATCAGCAATTGCGTGGCCGACTACTCCGTGCTGTAC
AACctgGCCcctTTCttcACCTTCAAGTGCTACGGCGTGTCCCCTACCAAGCTGAACGA
CCTGTGCTTCACAAACGTGTACGCCGACAGCTTCGTGATCCGGGGAGATGAAGT
GCGGCAGATTGCCCCTGGACAGACAGGCaacATCGCCGACTACAACTACAAGCTG
CCCGACGACTTCACCGGCTGTGTGATTGCCTGGAACAGCAACaagCTGGACTCCAA
AGTCagcGGCAACTACAATTACCTGTACCGGCTGTTCCGGAAGTCCAATCTGAAGC
CCTTCGAGCGGGACATCTCCACCGAGATCTATCAGGCCGGCaacaagCCTTGTAACG
GCGTGgccGGCTTCAACTGCTACTTCCCACTGcagTCCTACagcTTTcagCCCACAtacGG
CGTGGGCcacCAGCCCTACAGAGTGGTGGTGCTGAGCTTCGAACTGCTGCATGCCC
CTGCCACAGTGTGCGGCCCTAAGAAgAGCACCAATCTCGTGAAGAACAAATGCG
TGAACTTCAACTTCAACGGCCTGaagGGCACCGGCGTGCTGACAGAGAGCAACAA
GAAGTTCCTGCCATTCCAGCAGTTTGGCCGGGATATCGCCGATACCACAGACGCC
GTTAGAGATCCCCAGACACTGGAAATCCTGGACATCACCCCTTGCAGCTTCGGCG
GAGTGTCTGTGATCACCCCTGGCACCAACACCAGCAATCAGGTGGCAGTGCTGT
ACCAGggcGTGAACTGTACCGAAGTGCCCGTGGCCATTCACGCCGATCAGCTGAC
ACCTACATGGCGGGTGTACTCCACCGGCAGCAATGTGTTTCAGACCAGAGCCGG
CTGTCTGATCGGAGCCGAGtacGTGAACAATAGCTACGAGTGCGACATCCCCATCG
GCGCTGGCATCTGTGCCAGCTACCAGACACAGACAaagAGCcacGCCTCTGTGGCC
AGCCAGAGCATCATTGCCTACACAATGTCTCTGGGCGCCGAGAACAGCGTGGCC
TACTCCAACAACTCTATCGCTATCCCCACCAACTTCACCATCAGCGTGACCACAG
AGATCCTGCCTGTGTCCATGACCAAGACCAGCGTGGACTGCACCATGTACATCTG
CGGCGATTCCACCGAGTGCTCCAACCTGCTGCTGCAGTACGGCAGCTTCTGCACC
CAGCTGaagAGAGCCCTGACAGGGATCGCCGTGGAACAGGACAAGAACACCCAA
GAGGTGTTCGCCCAAGTGAAGCAGATCTACAAGACCCCTCCTATCAAGtacTTCGG
CGGCTTCAATTTCAGCCAGATTCTGCCCGATCCTAGCAAGCCCAGCAAGCGGAGC
cctATCGAGGACCTGCTGTTCAACAAAGTGACACTGGCCGACGCCGGCTTCATCAA
GCAGTATGGCGATTGTCTGGGCGACATTGCCGCCAGGGATCTGATTTGCGCCCAG
AAGTTTaagGGACTGACAGTGCTGCCTCCTCTGCTGACCGATGAGATGATCGCCCA
GTACACATCTGCCCTGCTGGCCGGCACAATCACAAGCGGCTGGACATTTGGAGCT
GGCcctGCTCTGCAGATCCCCTTTccaATGCAGATGGCCTACCGGTTCAACGGCATC
GGAGTGACCCAGAATGTGCTGTACGAGAACCAGAAGCTGATCGCCAACCAGTTC
AACAGCGCCATCGGCAAGATCCAGGACAGCCTGAGCAGCACAcccAGCGCCCTGG
GAAAGCTGCAGGACGTGGTCAACcacAATGCCCAGGCACTGAACACCCTGGTCAA
GCAGCTGTCCTCCaagTTCGGCGCCATCAGCTCTGTGCTGAACGATATCttcAGCAG
ACTGGACccccctGAAGCCGAGGTGCAGATCGACAGACTGATCACCGGAAGGCTGC
AGTCCCTGCAGACCTACGTTACCCAGCAGCTGATCAGAGCCGCCGAGATTAGAG
CCTCTGCCAATCTGGCCGCCACCAAGATGTCTGAGTGTGTGCTGGGCCAGAGCAA
GAGAGTGGACTTTTGCGGCAAGGGCTACCACCTGATGAGCTTCCCTCAGTCTGCC
CCTCACGGCGTGGTGTTTCTGCACGTGACATACGTGCCCGCTCAAGAGAAGAATT
TCACCACCGCTCCAGCCATCTGCCACGACGGCAAAGCCCACTTTCCTAGAGAAG
GCGTGTTCGTGTCCAACGGCACCCATTGGTTCGTGACCCAGCGGAACTTCTACGA
GCCCCAGATCATCACCACCGACAACACCTTCGTGTCTGGCAACTGCGACGTCGTG
ATCGGCATTGTGAACAATACCGTGTACGACCCTCTGCAGCCCGAGCTGGACAGCT
TCAAAGAGGAACTGGATAAGTACTTTAAGAACCACACAAGCCCCGACGTGGACC
TGGGCGATATCAGCGGAATCAATGCCAGCGTCGTGAACATCCAGAAAGAGATCG
ACCGGCTGAACGAGGTGGCCAAGAATCTGAACGAGAGCCTGATCGACCTGCAAG
AACTGGGGAAGTACGAGCAGTACATCAAGTGGCCCggcggaggtgggtcgCTCATAACA
TACATCGTCCTGACTATAATCAGCTTGGTATTTGGTATTTTGTCTTTGATTCTTGC
ATGCTATTTGATGTATAAACAGAAAGCTCAGCAGAAGACTCTCCTGTGGCTCGGT
AACAACACACTCGACCAGATGAGAGCAACTACAAAGATGTGA
Nucleotide sequence of Omicron BA.1 (Q493Q498): SEQ ID NO: 15 (HXP-S
ectodomain; GS linker sequence bold and underlined; transmembrane and cytoplasmic
domains of NDV F protein double underlined)
CAGTGTGTGAACCTGACCACAAGAACCCAGCTGCCTCCAGCCTACACCAACAGC
TTTACCAGAGGCGTGTACTACCCCGACAAGGTGTTCAGATCCAGCGTGCTGCACT
CTACCCAGGACCTGTTCCTGCCTTTCTTCAGCAACGTGACCTGGTTCCACgtgATCT
CCGGCACCAATGGCACCAAGAGATTCGACAACCCCGTGCTGCCCTTCAACGACG
GGGTGTACTTTGCCAGCatcGAGAAGTCCAACATCATCAGAGGCTGGATCTTCGGC
ACCACACTGGACAGCAAGACCCAGAGCCTGCTGATCGTGAACAACGCCACCAAC
GTGGTCATCAAAGTGTGCGAGTTCCAGTTCTGCAACGACCCCTTCCTGgacCACAA
GAACAACAAGAGCTGGATGGAAAGCGAGTTCCGGGTGTACAGCAGCGCCAACA
ACTGCACCTTCGAGTACGTGTCCCAGCCTTTCCTGATGGACCTGGAAGGCAAGCA
GGGCAACTTCAAGAACCTGCGCGAGTTCGTGTTCAAGAACATCGACGGCTACTTC
AAGATCTACAGCAAGCACACCCCTATCatcGTGCGGgaacccgaaGATCTGCCTCAGGG
CTTCTCTGCTCTGGAACCCCTGGTGGATCTGCCCATCGGCATCAACATCACCCGG
TTTCAGACACTGCTGGCCCTGCACAGAAGCTACCTGACACCTGGCGATAGCAGC
AGCGGATGGACAGCTGGTGCCGCCGCTTACTATGTGGGCTACCTGCAGCCTAGA
ACCTTTCTGCTGAAGTACAACGAGAACGGCACCATCACCGACGCCGTGGATTGT
GCTCTGGATCCTCTGAGCGAGACAAAGTGCACCCTGAAGTCCTTCACCGTGGAgA
AGGGCATCTACCAGACCAGCAACTTCCGGGTGCAGCCCACCGAATCCATCGTGC
GGTTCCCCAATATCACCAATCTGTGCCCCTTCgacGAGGTGTTCAATGCCACCAGA
TTCGCCTCTGTGTACGCCTGGAACCGGAAGCGGATCAGCAATTGCGTGGCCGACT
ACTCCGTGCTGTACAACctgGCCcctTTCttcACCTTCAAGTGCTACGGCGTGTCCCCT
ACCAAGCTGAACGACCTGTGCTTCACAAACGTGTACGCCGACAGCTTCGTGATCC
GGGGAGATGAAGTGCGGCAGATTGCCCCTGGACAGACAGGCaacATCGCCGACTA
CAACTACAAGCTGCCCGACGACTTCACCGGCTGTGTGATTGCCTGGAACAGCAA
CaagCTGGACTCCAAAGTCagcGGCAACTACAATTACCTGTACCGGCTGTTCCGGAA
GTCCAATCTGAAGCCCTTCGAGCGGGACATCTCCACCGAGATCTATCAGGCCGGC
aacaagCCTTGTAACGGCGTGgccGGCTTCAACTGCTACTTCCCACTGcagTCCTACagc
TTTcagCCCACAtacGGCGTGGGCcacCAGCCCTACAGAGTGGTGGTGCTGAGCTTCG
AACTGCTGCATGCCCCTGCCACAGTGTGCGGCCCTAAGAAgAGCACCAATCTCGT
GAAGAACAAATGCGTGAACTTCAACTTCAACGGCCTGaagGGCACCGGCGTGCTG
ACAGAGAGCAACAAGAAGTTCCTGCCATTCCAGCAGTTTGGCCGGGATATCGCC
GATACCACAGACGCCGTTAGAGATCCCCAGACACTGGAAATCCTGGACATCACC
CCTTGCAGCTTCGGCGGAGTGTCTGTGATCACCCCTGGCACCAACACCAGCAATC
AGGTGGCAGTGCTGTACCAGggcGTGAACTGTACCGAAGTGCCCGTGGCCATTCA
CGCCGATCAGCTGACACCTACATGGCGGGTGTACTCCACCGGCAGCAATGTGTTT
CAGACCAGAGCCGGCTGTCTGATCGGAGCCGAGtacGTGAACAATAGCTACGAGT
GCGACATCCCCATCGGCGCTGGCATCTGTGCCAGCTACCAGACACAGACAaagAG
CcacGCCTCTGTGGCCAGCCAGAGCATCATTGCCTACACAATGTCTCTGGGCGCCG
AGAACAGCGTGGCCTACTCCAACAACTCTATCGCTATCCCCACCAACTTCACCAT
CAGCGTGACCACAGAGATCCTGCCTGTGTCCATGACCAAGACCAGCGTGGACTG
CACCATGTACATCTGCGGCGATTCCACCGAGTGCTCCAACCTGCTGCTGCAGTAC
GGCAGCTTCTGCACCCAGCTGaagAGAGCCCTGACAGGGATCGCCGTGGAACAGG
ACAAGAACACCCAAGAGGTGTTCGCCCAAGTGAAGCAGATCTACAAGACCCCTC
CTATCAAGtacTTCGGCGGCTTCAATTTCAGCCAGATTCTGCCCGATCCTAGCAAG
CCCAGCAAGCGGAGCcctATCGAGGACCTGCTGTTCAACAAAGTGACACTGGCCG
ACGCCGGCTTCATCAAGCAGTATGGCGATTGTCTGGGCGACATTGCCGCCAGGG
ATCTGATTTGCGCCCAGAAGTTTaagGGACTGACAGTGCTGCCTCCTCTGCTGACC
GATGAGATGATCGCCCAGTACACATCTGCCCTGCTGGCCGGCACAATCACAAGC
GGCTGGACATTTGGAGCTGGCcctGCTCTGCAGATCCCCTTTccaATGCAGATGGCC
TACCGGTTCAACGGCATCGGAGTGACCCAGAATGTGCTGTACGAGAACCAGAAG
CTGATCGCCAACCAGTTCAACAGCGCCATCGGCAAGATCCAGGACAGCCTGAGC
AGCACAcccAGCGCCCTGGGAAAGCTGCAGGACGTGGTCAACcacAATGCCCAGGC
ACTGAACACCCTGGTCAAGCAGCTGTCCTCCaagTTCGGCGCCATCAGCTCTGTGC
TGAACGATATCttcAGCAGACTGGACccccctGAAGCCGAGGTGCAGATCGACAGACT
GATCACCGGAAGGCTGCAGTCCCTGCAGACCTACGTTACCCAGCAGCTGATCAG
AGCCGCCGAGATTAGAGCCTCTGCCAATCTGGCCGCCACCAAGATGTCTGAGTGT
GTGCTGGGCCAGAGCAAGAGAGTGGACTTTTGCGGCAAGGGCTACCACCTGATG
AGCTTCCCTCAGTCTGCCCCTCACGGCGTGGTGTTTCTGCACGTGACATACGTGC
CCGCTCAAGAGAAGAATTTCACCACCGCTCCAGCCATCTGCCACGACGGCAAAG
CCCACTTTCCTAGAGAAGGCGTGTTCGTGTCCAACGGCACCCATTGGTTCGTGAC
CCAGCGGAACTTCTACGAGCCCCAGATCATCACCACCGACAACACCTTCGTGTCT
GGCAACTGCGACGTCGTGATCGGCATTGTGAACAATACCGTGTACGACCCTCTGC
AGCCCGAGCTGGACAGCTTCAAAGAGGAACTGGATAAGTACTTTAAGAACCACA
CAAGCCCCGACGTGGACCTGGGCGATATCAGCGGAATCAATGCCAGCGTCGTGA
ACATCCAGAAAGAGATCGACCGGCTGAACGAGGTGGCCAAGAATCTGAACGAG
AGCCTGATCGACCTGCAAGAACTGGGGAAGTACGAGCAGTACATCAAGTGGCCC
ggcggaggtgggtcgCTCATAACATACATCGTCCTGACTATAATCAGCTTGGTATTTGGT
ATTTTGTCTTTGATTCTTGCATGCTATTTGATGTATAAACAGAAAGCTCAGCAGA
AGACTCTCCTGTGGCTCGGTAACAACACACTCGACCAGATGAGAGCAACTACAA
AGATGTGA
Amino acid sequence of Omicron BA.1 (Q493Q498): SEQ ID NO: 16 (signal peptide in
bold; HXP-S ectodomain; GS linker sequence bold and underlined; transmembrane
and cytoplasmic domains of NDV F protein double underlined)
MFVFLVLLPLVSSQCVNLTTRTQLPPAYTNSFTRGVYYPDKVFRSSVLHSTQDLFLP
FFSNVTWFHVISGTNGTKRFDNPVLPFNDGVYFASIEKSNIIRGWIFGTTLDSKTQSLL
IVNNATNVVIKVCEFQFCNDPFLDHKNNKSWMESEFRVYSSANNCTFEYVSQPFLM
DLEGKQGNFKNLREFVFKNIDGYFKIYSKHTPIIVREPEDLPQGFSALEPLVDLPIGINI
TRFQTLLALHRSYLTPGDSSSGWTAGAAAYYVGYLQPRTFLLKYNENGTITDAVDC
ALDPLSETKCTLKSFTVEKGIYQTSNFRVQPTESIVRFPNITNLCPFDEVFNATRFASV
YAWNRKRISNCVADYSVLYNLAPFFTFKCYGVSPTKLNDLCFTNVYADSFVIRGDEV
RQIAPGQTGNIADYNYKLPDDFTGCVIAWNSNKLDSKVSGNYNYLYRLFRKSNLKPF
ERDISTEIYQAGNKPCNGVAGFNCYFPLQSYSFQPTYGVGHQPYRVVVLSFELLHAP
ATVCGPKKSTNLVKNKCVNFNFNGLKGTGVLTESNKKFLPFQQFGRDIADTTDAVR
DPQTLEILDITPCSFGGVSVITPGTNTSNQVAVLYQGVNCTEVPVAIHADQLTPTWRV
YSTGSNVFQTRAGCLIGAEYVNNSYECDIPIGAGICASYQTQTKSHASVASQSIIAYT
MSLGAENSVAYSNNSIAIPTNFTISVTTEILPVSMTKTSVDCTMYICGDSTECSNLLLQ
YGSFCTQLKRALTGIAVEQDKNTQEVFAQVKQIYKTPPIKYFGGFNFSQILPDPSKPS
KRSPIEDLLFNKVTLADAGFIKQYGDCLGDIAARDLICAQKFKGLTVLPPLLTDEMIA
QYTSALLAGTITSGWTFGAGPALQIPFPMQMAYRENGIGVTQNVLYENQKLIANQFN
SAIGKIQDSLSSTPSALGKLQDVVNHNAQALNTLVKQLSSKFGAISSVLNDIFSRLDPP
EAEVQIDRLITGRLQSLQTYVTQQLIRAAEIRASANLAATKMSECVLGQSKRVDFCG
KGYHLMSFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKEELDKYFKN
HTSPDVDLGDISGINASVVNIQKEIDRLNEVAKNLNESLIDLQELGKYEQYIKWPGGG
GSLITYIVLTIISLVFGILSLILACYLMYKQKAQQKTLLWLGNNTLDQMRATTKM*
Amino acid sequence of Omicron BA.1 (Q493Q498): SEQ ID NO: 17 (HXP-S
ectodomain; GS linker sequence bold and underlined; transmembrane and cytoplasmic
domains of NDV F protein double underlined)
QCVNLTTRTQLPPAYTNSFTRGVYYPDKVFRSSVLHSTQDLFLPFFSNVTWFHVISGT
NGTKRFDNPVLPFNDGVYFASIEKSNIIRGWIFGTTLDSKTQSLLIVNNATNVVIKVCE
FQFCNDPFLDHKNNKSWMESEFRVYSSANNCTFEYVSQPFLMDLEGKQGNFKNLRE
FVFKNIDGYFKIYSKHTPIIVREPEDLPQGFSALEPLVDLPIGINITRFQTLLALHRSYLT
PGDSSSGWTAGAAAYYVGYLQPRTFLLKYNENGTITDAVDCALDPLSETKCTLKSFT
VEKGIYQTSNFRVQPTESIVRFPNITNLCPFDEVFNATRFASVYAWNRKRISNCVADY
SVLYNLAPFFTFKCYGVSPTKLNDLCFTNVYADSFVIRGDEVRQIAPGQTGNIADYN
YKLPDDFTGCVIAWNSNKLDSKVSGNYNYLYRLFRKSNLKPFERDISTEIYQAGNKP
CNGVAGFNCYFPLQSYSFQPTYGVGHQPYRVVVLSFELLHAPATVCGPKKSTNLVK
NKCVNFNFNGLKGTGVLTESNKKFLPFQQFGRDIADTTDAVRDPQTLEILDITPCSFG
GVSVITPGTNTSNQVAVLYQGVNCTEVPVAIHADQLTPTWRVYSTGSNVFQTRAGC
LIGAEYVNNSYECDIPIGAGICASYQTQTKSHASVASQSIIAYTMSLGAENSVAYSNN
SIAIPTNFTISVTTEILPVSMTKTSVDCTMYICGDSTECSNLLLQYGSFCTQLKRALTGI
AVEQDKNTQEVFAQVKQIYKTPPIKYFGGFNFSQILPDPSKPSKRSPIEDLLFNKVTLA
DAGFIKQYGDCLGDIAARDLICAQKFKGLTVLPPLLTDEMIAQYTSALLAGTITSGWT
FGAGPALQIPFPMQMAYRENGIGVTQNVLYENQKLIANQFNSAIGKIQDSLSSTPSAL
GKLQDVVNHNAQALNTLVKQLSSKFGAISSVLNDIFSRLDPPEAEVQIDRLITGRLQS
LQTYVTQQLIRAAEIRASANLAATKMSECVLGQSKRVDFCGKGYHLMSFPQSAPHG
VVFLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISGINA
SVVNIQKEIDRLNEVAKNLNESLIDLQELGKYEQYIKWPGGGGSLITYIVLTIISLVFGI
LSLILACYLMYKQKAQQKTLLWLGNNTLDQMRATTKM*
Nucleotide sequence of Omicron BA.1 Spike Protein Ectodomain: SEQ ID NO: 18
CAGTGTGTGAACCTGACCACAAGAACCCAGCTGCCTCCAGCCTACACCAACAGC
TTTACCAGAGGCGTGTACTACCCCGACAAGGTGTTCAGATCCAGCGTGCTGCACT
CTACCCAGGACCTGTTCCTGCCTTTCTTCAGCAACGTGACCTGGTTCCACgtgATCT
CCGGCACCAATGGCACCAAGAGATTCGACAACCCCGTGCTGCCCTTCAACGACG
GGGTGTACTTTGCCAGCatcGAGAAGTCCAACATCATCAGAGGCTGGATCTTCGGC
ACCACACTGGACAGCAAGACCCAGAGCCTGCTGATCGTGAACAACGCCACCAAC
GTGGTCATCAAAGTGTGCGAGTTCCAGTTCTGCAACGACCCCTTCCTGgacCACAA
GAACAACAAGAGCTGGATGGAAAGCGAGTTCCGGGTGTACAGCAGCGCCAACA
ACTGCACCTTCGAGTACGTGTCCCAGCCTTTCCTGATGGACCTGGAAGGCAAGCA
GGGCAACTTCAAGAACCTGCGCGAGTTCGTGTTCAAGAACATCGACGGCTACTTC
AAGATCTACAGCAAGCACACCCCTATCatcGTGCGGgaacccgaaGATCTGCCTCAGGG
CTTCTCTGCTCTGGAACCCCTGGTGGATCTGCCCATCGGCATCAACATCACCCGG
TTTCAGACACTGCTGGCCCTGCACAGAAGCTACCTGACACCTGGCGATAGCAGC
AGCGGATGGACAGCTGGTGCCGCCGCTTACTATGTGGGCTACCTGCAGCCTAGA
ACCTTTCTGCTGAAGTACAACGAGAACGGCACCATCACCGACGCCGTGGATTGT
GCTCTGGATCCTCTGAGCGAGACAAAGTGCACCCTGAAGTCCTTCACCGTGGAgA
AGGGCATCTACCAGACCAGCAACTTCCGGGTGCAGCCCACCGAATCCATCGTGC
GGTTCCCCAATATCACCAATCTGTGCCCCTTCgacGAGGTGTTCAATGCCACCAGA
TTCGCCTCTGTGTACGCCTGGAACCGGAAGCGGATCAGCAATTGCGTGGCCGACT
ACTCCGTGCTGTACAACctgGCCcctTTCttcACCTTCAAGTGCTACGGCGTGTCCCCT
ACCAAGCTGAACGACCTGTGCTTCACAAACGTGTACGCCGACAGCTTCGTGATCC
GGGGAGATGAAGTGCGGCAGATTGCCCCTGGACAGACAGGCaacATCGCCGACTA
CAACTACAAGCTGCCCGACGACTTCACCGGCTGTGTGATTGCCTGGAACAGCAA
CaagCTGGACTCCAAAGTCagcGGCAACTACAATTACCTGTACCGGCTGTTCCGGAA
GTCCAATCTGAAGCCCTTCGAGCGGGACATCTCCACCGAGATCTATCAGGCCGGC
aacaagCCTTGTAACGGCGTGgccGGCTTCAACTGCTACTTCCCACTGagaTCCTACagc
TTTagaCCCACAtacGGCGTGGGCcacCAGCCCTACAGAGTGGTGGTGCTGAGCTTCG
AACTGCTGCATGCCCCTGCCACAGTGTGCGGCCCTAAGAAgAGCACCAATCTCGT
GAAGAACAAATGCGTGAACTTCAACTTCAACGGCCTGaagGGCACCGGCGTGCTG
ACAGAGAGCAACAAGAAGTTCCTGCCATTCCAGCAGTTTGGCCGGGATATCGCC
GATACCACAGACGCCGTTAGAGATCCCCAGACACTGGAAATCCTGGACATCACC
CCTTGCAGCTTCGGCGGAGTGTCTGTGATCACCCCTGGCACCAACACCAGCAATC
AGGTGGCAGTGCTGTACCAGggcGTGAACTGTACCGAAGTGCCCGTGGCCATTCA
CGCCGATCAGCTGACACCTACATGGCGGGTGTACTCCACCGGCAGCAATGTGTTT
CAGACCAGAGCCGGCTGTCTGATCGGAGCCGAGtacGTGAACAATAGCTACGAGT
GCGACATCCCCATCGGCGCTGGCATCTGTGCCAGCTACCAGACACAGACAaagAG
CcacGCCTCTGTGGCCAGCCAGAGCATCATTGCCTACACAATGTCTCTGGGCGCCG
AGAACAGCGTGGCCTACTCCAACAACTCTATCGCTATCCCCACCAACTTCACCAT
CAGCGTGACCACAGAGATCCTGCCTGTGTCCATGACCAAGACCAGCGTGGACTG
CACCATGTACATCTGCGGCGATTCCACCGAGTGCTCCAACCTGCTGCTGCAGTAC
GGCAGCTTCTGCACCCAGCTGaagAGAGCCCTGACAGGGATCGCCGTGGAACAGG
ACAAGAACACCCAAGAGGTGTTCGCCCAAGTGAAGCAGATCTACAAGACCCCTC
CTATCAAGtacTTCGGCGGCTTCAATTTCAGCCAGATTCTGCCCGATCCTAGCAAG
CCCAGCAAGCGGAGCcctATCGAGGACCTGCTGTTCAACAAAGTGACACTGGCCG
ACGCCGGCTTCATCAAGCAGTATGGCGATTGTCTGGGCGACATTGCCGCCAGGG
ATCTGATTTGCGCCCAGAAGTTTaagGGACTGACAGTGCTGCCTCCTCTGCTGACC
GATGAGATGATCGCCCAGTACACATCTGCCCTGCTGGCCGGCACAATCACAAGC
GGCTGGACATTTGGAGCTGGCcctGCTCTGCAGATCCCCTTTccaATGCAGATGGCC
TACCGGTTCAACGGCATCGGAGTGACCCAGAATGTGCTGTACGAGAACCAGAAG
CTGATCGCCAACCAGTTCAACAGCGCCATCGGCAAGATCCAGGACAGCCTGAGC
AGCACAcccAGCGCCCTGGGAAAGCTGCAGGACGTGGTCAACcacAATGCCCAGGC
ACTGAACACCCTGGTCAAGCAGCTGTCCTCCaagTTCGGCGCCATCAGCTCTGTGC
TGAACGATATCttcAGCAGACTGGACccccctGAAGCCGAGGTGCAGATCGACAGACT
GATCACCGGAAGGCTGCAGTCCCTGCAGACCTACGTTACCCAGCAGCTGATCAG
AGCCGCCGAGATTAGAGCCTCTGCCAATCTGGCCGCCACCAAGATGTCTGAGTGT
GTGCTGGGCCAGAGCAAGAGAGTGGACTTTTGCGGCAAGGGCTACCACCTGATG
AGCTTCCCTCAGTCTGCCCCTCACGGCGTGGTGTTTCTGCACGTGACATACGTGC
CCGCTCAAGAGAAGAATTTCACCACCGCTCCAGCCATCTGCCACGACGGCAAAG
CCCACTTTCCTAGAGAAGGCGTGTTCGTGTCCAACGGCACCCATTGGTTCGTGAC
CCAGCGGAACTTCTACGAGCCCCAGATCATCACCACCGACAACACCTTCGTGTCT
GGCAACTGCGACGTCGTGATCGGCATTGTGAACAATACCGTGTACGACCCTCTGC
AGCCCGAGCTGGACAGCTTCAAAGAGGAACTGGATAAGTACTTTAAGAACCACA
CAAGCCCCGACGTGGACCTGGGCGATATCAGCGGAATCAATGCCAGCGTCGTGA
ACATCCAGAAAGAGATCGACCGGCTGAACGAGGTGGCCAAGAATCTGAACGAG
AGCCTGATCGACCTGCAAGAACTGGGGAAGTACGAGCAGTACATCAAGTGGCCC
Amino acid sequence of Omicron BA.1 Spike Protein Ectodomain: SEQ ID NO: 19
QCVNLTTRTQLPPAYTNSFTRGVYYPDKVFRSSVLHSTQDLFLPFFSNVTWFHVISGT
NGTKRFDNPVLPFNDGVYFASIEKSNIIRGWIFGTTLDSKTQSLLIVNNATNVVIKVCE
FQFCNDPFLDHKNNKSWMESEFRVYSSANNCTFEYVSQPFLMDLEGKQGNFKNLRE
FVFKNIDGYFKIYSKHTPIIVREPEDLPQGFSALEPLVDLPIGINITRFQTLLALHRSYLT
PGDSSSGWTAGAAAYYVGYLQPRTFLLKYNENGTITDAVDCALDPLSETKCTLKSFT
VEKGIYQTSNFRVQPTESIVRFPNITNLCPFDEVFNATRFASVYAWNRKRISNCVADY
SVLYNLAPFFTFKCYGVSPTKLNDLCFTNVYADSFVIRGDEVRQIAPGQTGNIADYN
YKLPDDFTGCVIAWNSNKLDSKVSGNYNYLYRLFRKSNLKPFERDISTEIYQAGNKP
CNGVAGFNCYFPLRSYSFRPTYGVGHQPYRVVVLSFELLHAPATVCGPKKSTNLVK
NKCVNFNFNGLKGTGVLTESNKKFLPFQQFGRDIADTTDAVRDPQTLEILDITPCSFG
GVSVITPGTNTSNQVAVLYQGVNCTEVPVAIHADQLTPTWRVYSTGSNVFQTRAGC
LIGAEYVNNSYECDIPIGAGICASYQTQTKSHASVASQSIIAYTMSLGAENSVAYSNN
SIAIPTNFTISVTTEILPVSMTKTSVDCTMYICGDSTECSNLLLQYGSFCTQLKRALTGI
AVEQDKNTQEVFAQVKQIYKTPPIKYFGGFNFSQILPDPSKPSKRSPIEDLLFNKVTLA
DAGFIKQYGDCLGDIAARDLICAQKFKGLTVLPPLLTDEMIAQYTSALLAGTITSGWT
FGAGPALQIPFPMQMAYRFNGIGVTQNVLYENQKLIANQFNSAIGKIQDSLSSTPSAL
GKLQDVVNHNAQALNTLVKQLSSKFGAISSVLNDIFSRLDPPEAEVQIDRLITGRLQS
LQTYVTQQLIRAAEIRASANLAATKMSECVLGQSKRVDFCGKGYHLMSFPQSAPHG
VVFLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISGINA
SVVNIQKEIDRLNEVAKNLNESLIDLQELGKYEQYIKWP
Nucleotide sequence of Omicron BA.1 (H655_deCSV6871) Spike Protein Ectodomain:
SEQ ID NO: 20
CAGTGTGTGAACCTGACCACAAGAACCCAGCTGCCTCCAGCCTACACCAACAGC
TTTACCAGAGGCGTGTACTACCCCGACAAGGTGTTCAGATCCAGCGTGCTGCACT
CTACCCAGGACCTGTTCCTGCCTTTCTTCAGCAACGTGACCTGGTTCCACgtgATCT
CCGGCACCAATGGCACCAAGAGATTCGACAACCCCGTGCTGCCCTTCAACGACG
GGGTGTACTTTGCCAGCatcGAGAAGTCCAACATCATCAGAGGCTGGATCTTCGGC
ACCACACTGGACAGCAAGACCCAGAGCCTGCTGATCGTGAACAACGCCACCAAC
GTGGTCATCAAAGTGTGCGAGTTCCAGTTCTGCAACGACCCCTTCCTGgacCACAA
GAACAACAAGAGCTGGATGGAAAGCGAGTTCCGGGTGTACAGCAGCGCCAACA
ACTGCACCTTCGAGTACGTGTCCCAGCCTTTCCTGATGGACCTGGAAGGCAAGCA
GGGCAACTTCAAGAACCTGCGCGAGTTCGTGTTCAAGAACATCGACGGCTACTTC
AAGATCTACAGCAAGCACACCCCTATCatcGTGCGGgaacccgaaGATCTGCCTCAGGG
CTTCTCTGCTCTGGAACCCCTGGTGGATCTGCCCATCGGCATCAACATCACCCGG
TTTCAGACACTGCTGGCCCTGCACAGAAGCTACCTGACACCTGGCGATAGCAGC
AGCGGATGGACAGCTGGTGCCGCCGCTTACTATGTGGGCTACCTGCAGCCTAGA
ACCTTTCTGCTGAAGTACAACGAGAACGGCACCATCACCGACGCCGTGGATTGT
GCTCTGGATCCTCTGAGCGAGACAAAGTGCACCCTGAAGTCCTTCACCGTGGAgA
AGGGCATCTACCAGACCAGCAACTTCCGGGTGCAGCCCACCGAATCCATCGTGC
GGTTCCCCAATATCACCAATCTGTGCCCCTTCgacGAGGTGTTCAATGCCACCAGA
TTCGCCTCTGTGTACGCCTGGAACCGGAAGCGGATCAGCAATTGCGTGGCCGACT
ACTCCGTGCTGTACAACctgGCCcctTTCttcACCTTCAAGTGCTACGGCGTGTCCCCT
ACCAAGCTGAACGACCTGTGCTTCACAAACGTGTACGCCGACAGCTTCGTGATCC
GGGGAGATGAAGTGCGGCAGATTGCCCCTGGACAGACAGGCaacATCGCCGACTA
CAACTACAAGCTGCCCGACGACTTCACCGGCTGTGTGATTGCCTGGAACAGCAA
CaagCTGGACTCCAAAGTCagcGGCAACTACAATTACCTGTACCGGCTGTTCCGGAA
GTCCAATCTGAAGCCCTTCGAGCGGGACATCTCCACCGAGATCTATCAGGCCGGC
aacaagCCTTGTAACGGCGTGgccGGCTTCAACTGCTACTTCCCACTGagaTCCTACagc
TTTagaCCCACAtacGGCGTGGGCcacCAGCCCTACAGAGTGGTGGTGCTGAGCTTCG
AACTGCTGCATGCCCCTGCCACAGTGTGCGGCCCTAAGAAgAGCACCAATCTCGT
GAAGAACAAATGCGTGAACTTCAACTTCAACGGCCTGaagGGCACCGGCGTGCTG
ACAGAGAGCAACAAGAAGTTCCTGCCATTCCAGCAGTTTGGCCGGGATATCGCC
GATACCACAGACGCCGTTAGAGATCCCCAGACACTGGAAATCCTGGACATCACC
CCTTGCAGCTTCGGCGGAGTGTCTGTGATCACCCCTGGCACCAACACCAGCAATC
AGGTGGCAGTGCTGTACCAGggcGTGAACTGTACCGAAGTGCCCGTGGCCATTCA
CGCCGATCAGCTGACACCTACATGGCGGGTGTACTCCACCGGCAGCAATGTGTTT
CAGACCAGAGCCGGCTGTCTGATCGGAGCCGAGcacGTGAACAATAGCTACGAGT
GCGACATCCCCATCGGCGCTGGCATCTGTGCCAGCTACCAGACACAGACAatcGCC
AGCCAGAGCATCATTGCCTACACAATGTCTCTGGGCGCCGAGAACAGCGTGGCC
TACTCCAACAACTCTATCGCTATCCCCACCAACTTCACCATCAGCGTGACCACAG
AGATCCTGCCTGTGTCCATGACCAAGACCAGCGTGGACTGCACCATGTACATCTG
CGGCGATTCCACCGAGTGCTCCAACCTGCTGCTGCAGTACGGCAGCTTCTGCACC
CAGCTGaagAGAGCCCTGACAGGGATCGCCGTGGAACAGGACAAGAACACCCAA
GAGGTGTTCGCCCAAGTGAAGCAGATCTACAAGACCCCTCCTATCAAGtacTTCGG
CGGCTTCAATTTCAGCCAGATTCTGCCCGATCCTAGCAAGCCCAGCAAGCGGAGC
cctATCGAGGACCTGCTGTTCAACAAAGTGACACTGGCCGACGCCGGCTTCATCAA
GCAGTATGGCGATTGTCTGGGCGACATTGCCGCCAGGGATCTGATTTGCGCCCAG
AAGTTTaagGGACTGACAGTGCTGCCTCCTCTGCTGACCGATGAGATGATCGCCCA
GTACACATCTGCCCTGCTGGCCGGCACAATCACAAGCGGCTGGACATTTGGAGCT
GGCcctGCTCTGCAGATCCCCTTTccaATGCAGATGGCCTACCGGTTCAACGGCATC
GGAGTGACCCAGAATGTGCTGTACGAGAACCAGAAGCTGATCGCCAACCAGTTC
AACAGCGCCATCGGCAAGATCCAGGACAGCCTGAGCAGCACAcccAGCGCCCTGG
GAAAGCTGCAGGACGTGGTCAACcacAATGCCCAGGCACTGAACACCCTGGTCAA
GCAGCTGTCCTCCaagTTCGGCGCCATCAGCTCTGTGCTGAACGATATCttcAGCAG
ACTGGACccccctGAAGCCGAGGTGCAGATCGACAGACTGATCACCGGAAGGCTGC
AGTCCCTGCAGACCTACGTTACCCAGCAGCTGATCAGAGCCGCCGAGATTAGAG
CCTCTGCCAATCTGGCCGCCACCAAGATGTCTGAGTGTGTGCTGGGCCAGAGCAA
GAGAGTGGACTTTTGCGGCAAGGGCTACCACCTGATGAGCTTCCCTCAGTCTGCC
CCTCACGGCGTGGTGTTTCTGCACGTGACATACGTGCCCGCTCAAGAGAAGAATT
TCACCACCGCTCCAGCCATCTGCCACGACGGCAAAGCCCACTTTCCTAGAGAAG
GCGTGTTCGTGTCCAACGGCACCCATTGGTTCGTGACCCAGCGGAACTTCTACGA
GCCCCAGATCATCACCACCGACAACACCTTCGTGTCTGGCAACTGCGACGTCGTG
ATCGGCATTGTGAACAATACCGTGTACGACCCTCTGCAGCCCGAGCTGGACAGCT
TCAAAGAGGAACTGGATAAGTACTTTAAGAACCACACAAGCCCCGACGTGGACC
TGGGCGATATCAGCGGAATCAATGCCAGCGTCGTGAACATCCAGAAAGAGATCG
ACCGGCTGAACGAGGTGGCCAAGAATCTGAACGAGAGCCTGATCGACCTGCAAG
AACTGGGGAAGTACGAGCAGTACATCAAGTGGCCC
Amino acid sequence of Omicron BA.1 (H655_deCSV6871) Spike Protein Ectodomain:
SEQ ID NO: 21
QCVNLTTRTQLPPAYTNSFTRGVYYPDKVFRSSVLHSTQDLFLPFFSNVTWFHVISGT
NGTKRFDNPVLPFNDGVYFASIEKSNIIRGWIFGTTLDSKTQSLLIVNNATNVVIKVCE
FQFCNDPFLDHKNNKSWMESEFRVYSSANNCTFEYVSQPFLMDLEGKQGNFKNLRE
FVFKNIDGYFKIYSKHTPIIVREPEDLPQGFSALEPLVDLPIGINITRFQTLLALHRSYLT
PGDSSSGWTAGAAAYYVGYLQPRTFLLKYNENGTITDAVDCALDPLSETKCTLKSFT
VEKGIYQTSNFRVQPTESIVRFPNITNLCPFDEVFNATRFASVYAWNRKRISNCVADY
SVLYNLAPFFTFKCYGVSPTKLNDLCFTNVYADSFVIRGDEVRQIAPGQTGNIADYN
YKLPDDFTGCVIAWNSNKLDSKVSGNYNYLYRLFRKSNLKPFERDISTEIYQAGNKP
CNGVAGFNCYFPLRSYSFRPTYGVGHQPYRVVVLSFELLHAPATVCGPKKSTNLVK
NKCVNFNFNGLKGTGVLTESNKKFLPFQQFGRDIADTTDAVRDPQTLEILDITPCSFG
GVSVITPGTNTSNQVAVLYQGVNCTEVPVAIHADQLTPTWRVYSTGSNVFQTRAGC
LIGAEHVNNSYECDIPIGAGICASYQTQTIASQSIIAYTMSLGAENSVAYSNNSIAIPTN
FTISVTTEILPVSMTKTSVDCTMYICGDSTECSNLLLQYGSFCTQLKRALTGIAVEQD
KNTQEVFAQVKQIYKTPPIKYFGGFNFSQILPDPSKPSKRSPIEDLLFNKVTLADAGFI
KQYGDCLGDIAARDLICAQKFKGLTVLPPLLTDEMIAQYTSALLAGTITSGWTFGAG
PALQIPFPMQMAYRENGIGVTQNVLYENQKLIANQFNSAIGKIQDSLSSTPSALGKLQ
DVVNHNAQALNTLVKQLSSKFGAISSVLNDIFSRLDPPEAEVQIDRLITGRLQSLQTY
VTQQLIRAAEIRASANLAATKMSECVLGQSKRVDFCGKGYHLMSFPQSAPHGVVFL
HVTYVPAQEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASVVN
IQKEIDRLNEVAKNLNESLIDLQELGKYEQYIKWP
Nucleotide sequence of Omicron BA.1 (Q493Q498) Spike Protein Ectodomain:
SEQ ID NO: 22
CAGTGTGTGAACCTGACCACAAGAACCCAGCTGCCTCCAGCCTACACCAACAGC
TTTACCAGAGGCGTGTACTACCCCGACAAGGTGTTCAGATCCAGCGTGCTGCACT
CTACCCAGGACCTGTTCCTGCCTTTCTTCAGCAACGTGACCTGGTTCCACgtgATCT
CCGGCACCAATGGCACCAAGAGATTCGACAACCCCGTGCTGCCCTTCAACGACG
GGGTGTACTTTGCCAGCatcGAGAAGTCCAACATCATCAGAGGCTGGATCTTCGGC
ACCACACTGGACAGCAAGACCCAGAGCCTGCTGATCGTGAACAACGCCACCAAC
GTGGTCATCAAAGTGTGCGAGTTCCAGTTCTGCAACGACCCCTTCCTGgacCACAA
GAACAACAAGAGCTGGATGGAAAGCGAGTTCCGGGTGTACAGCAGCGCCAACA
ACTGCACCTTCGAGTACGTGTCCCAGCCTTTCCTGATGGACCTGGAAGGCAAGCA
GGGCAACTTCAAGAACCTGCGCGAGTTCGTGTTCAAGAACATCGACGGCTACTTC
AAGATCTACAGCAAGCACACCCCTATCatcGTGCGGgaacccgaaGATCTGCCTCAGGG
CTTCTCTGCTCTGGAACCCCTGGTGGATCTGCCCATCGGCATCAACATCACCCGG
TTTCAGACACTGCTGGCCCTGCACAGAAGCTACCTGACACCTGGCGATAGCAGC
AGCGGATGGACAGCTGGTGCCGCCGCTTACTATGTGGGCTACCTGCAGCCTAGA
ACCTTTCTGCTGAAGTACAACGAGAACGGCACCATCACCGACGCCGTGGATTGT
GCTCTGGATCCTCTGAGCGAGACAAAGTGCACCCTGAAGTCCTTCACCGTGGAgA
AGGGCATCTACCAGACCAGCAACTTCCGGGTGCAGCCCACCGAATCCATCGTGC
GGTTCCCCAATATCACCAATCTGTGCCCCTTCgacGAGGTGTTCAATGCCACCAGA
TTCGCCTCTGTGTACGCCTGGAACCGGAAGCGGATCAGCAATTGCGTGGCCGACT
ACTCCGTGCTGTACAACctgGCCcctTTCttcACCTTCAAGTGCTACGGCGTGTCCCCT
ACCAAGCTGAACGACCTGTGCTTCACAAACGTGTACGCCGACAGCTTCGTGATCC
GGGGAGATGAAGTGCGGCAGATTGCCCCTGGACAGACAGGCaacATCGCCGACTA
CAACTACAAGCTGCCCGACGACTTCACCGGCTGTGTGATTGCCTGGAACAGCAA
CaagCTGGACTCCAAAGTCagcGGCAACTACAATTACCTGTACCGGCTGTTCCGGAA
GTCCAATCTGAAGCCCTTCGAGCGGGACATCTCCACCGAGATCTATCAGGCCGGC
aacaagCCTTGTAACGGCGTGgccGGCTTCAACTGCTACTTCCCACTGcagTCCTACagc
TTTcagCCCACAtacGGCGTGGGCcacCAGCCCTACAGAGTGGTGGTGCTGAGCTTCG
AACTGCTGCATGCCCCTGCCACAGTGTGCGGCCCTAAGAAgAGCACCAATCTCGT
GAAGAACAAATGCGTGAACTTCAACTTCAACGGCCTGaagGGCACCGGCGTGCTG
ACAGAGAGCAACAAGAAGTTCCTGCCATTCCAGCAGTTTGGCCGGGATATCGCC
GATACCACAGACGCCGTTAGAGATCCCCAGACACTGGAAATCCTGGACATCACC
CCTTGCAGCTTCGGCGGAGTGTCTGTGATCACCCCTGGCACCAACACCAGCAATC
AGGTGGCAGTGCTGTACCAGggcGTGAACTGTACCGAAGTGCCCGTGGCCATTCA
CGCCGATCAGCTGACACCTACATGGCGGGTGTACTCCACCGGCAGCAATGTGTTT
CAGACCAGAGCCGGCTGTCTGATCGGAGCCGAGtacGTGAACAATAGCTACGAGT
GCGACATCCCCATCGGCGCTGGCATCTGTGCCAGCTACCAGACACAGACAaagAG
CcacGCCTCTGTGGCCAGCCAGAGCATCATTGCCTACACAATGTCTCTGGGCGCCG
AGAACAGCGTGGCCTACTCCAACAACTCTATCGCTATCCCCACCAACTTCACCAT
CAGCGTGACCACAGAGATCCTGCCTGTGTCCATGACCAAGACCAGCGTGGACTG
CACCATGTACATCTGCGGCGATTCCACCGAGTGCTCCAACCTGCTGCTGCAGTAC
GGCAGCTTCTGCACCCAGCTGaagAGAGCCCTGACAGGGATCGCCGTGGAACAGG
ACAAGAACACCCAAGAGGTGTTCGCCCAAGTGAAGCAGATCTACAAGACCCCTC
CTATCAAGtacTTCGGCGGCTTCAATTTCAGCCAGATTCTGCCCGATCCTAGCAAG
CCCAGCAAGCGGAGCcctATCGAGGACCTGCTGTTCAACAAAGTGACACTGGCCG
ACGCCGGCTTCATCAAGCAGTATGGCGATTGTCTGGGCGACATTGCCGCCAGGG
ATCTGATTTGCGCCCAGAAGTTTaagGGACTGACAGTGCTGCCTCCTCTGCTGACC
GATGAGATGATCGCCCAGTACACATCTGCCCTGCTGGCCGGCACAATCACAAGC
GGCTGGACATTTGGAGCTGGCcctGCTCTGCAGATCCCCTTTccaATGCAGATGGCC
TACCGGTTCAACGGCATCGGAGTGACCCAGAATGTGCTGTACGAGAACCAGAAG
CTGATCGCCAACCAGTTCAACAGCGCCATCGGCAAGATCCAGGACAGCCTGAGC
AGCACAcccAGCGCCCTGGGAAAGCTGCAGGACGTGGTCAACcacAATGCCCAGGC
ACTGAACACCCTGGTCAAGCAGCTGTCCTCCaagTTCGGCGCCATCAGCTCTGTGC
TGAACGATATCttcAGCAGACTGGACccccctGAAGCCGAGGTGCAGATCGACAGACT
GATCACCGGAAGGCTGCAGTCCCTGCAGACCTACGTTACCCAGCAGCTGATCAG
AGCCGCCGAGATTAGAGCCTCTGCCAATCTGGCCGCCACCAAGATGTCTGAGTGT
GTGCTGGGCCAGAGCAAGAGAGTGGACTTTTGCGGCAAGGGCTACCACCTGATG
AGCTTCCCTCAGTCTGCCCCTCACGGCGTGGTGTTTCTGCACGTGACATACGTGC
CCGCTCAAGAGAAGAATTTCACCACCGCTCCAGCCATCTGCCACGACGGCAAAG
CCCACTTTCCTAGAGAAGGCGTGTTCGTGTCCAACGGCACCCATTGGTTCGTGAC
CCAGCGGAACTTCTACGAGCCCCAGATCATCACCACCGACAACACCTTCGTGTCT
GGCAACTGCGACGTCGTGATCGGCATTGTGAACAATACCGTGTACGACCCTCTGC
AGCCCGAGCTGGACAGCTTCAAAGAGGAACTGGATAAGTACTTTAAGAACCACA
CAAGCCCCGACGTGGACCTGGGCGATATCAGCGGAATCAATGCCAGCGTCGTGA
ACATCCAGAAAGAGATCGACCGGCTGAACGAGGTGGCCAAGAATCTGAACGAG
AGCCTGATCGACCTGCAAGAACTGGGGAAGTACGAGCAGTACATCAAGTGGCCC
Amino acid sequence of Omicron BA.1 (Q493Q498) Spike Protein Ectodomain:
SEQ ID NO: 23
QCVNLTTRTQLPPAYTNSFTRGVYYPDKVFRSSVLHSTQDLFLPFFSNVTWFHVISGT
NGTKRFDNPVLPFNDGVYFASIEKSNIIRGWIFGTTLDSKTQSLLIVNNATNVVIKVCE
FQFCNDPFLDHKNNKSWMESEFRVYSSANNCTFEYVSQPFLMDLEGKQGNFKNLRE
FVFKNIDGYFKIYSKHTPIIVREPEDLPQGFSALEPLVDLPIGINITRFQTLLALHRSYLT
PGDSSSGWTAGAAAYYVGYLQPRTFLLKYNENGTITDAVDCALDPLSETKCTLKSFT
VEKGIYQTSNFRVQPTESIVRFPNITNLCPFDEVFNATRFASVYAWNRKRISNCVADY
SVLYNLAPFFTFKCYGVSPTKLNDLCFTNVYADSFVIRGDEVRQIAPGQTGNIADYN
YKLPDDFTGCVIAWNSNKLDSKVSGNYNYLYRLFRKSNLKPFERDISTEIYQAGNKP
CNGVAGFNCYFPLQSYSFQPTYGVGHQPYRVVVLSFELLHAPATVCGPKKSTNLVK
NKCVNFNFNGLKGTGVLTESNKKFLPFQQFGRDIADTTDAVRDPQTLEILDITPCSFG
GVSVITPGTNTSNQVAVLYQGVNCTEVPVAIHADQLTPTWRVYSTGSNVFQTRAGC
LIGAEYVNNSYECDIPIGAGICASYQTQTKSHASVASQSIIAYTMSLGAENSVAYSNN
SIAIPTNFTISVTTEILPVSMTKTSVDCTMYICGDSTECSNLLLQYGSFCTQLKRALTGI
AVEQDKNTQEVFAQVKQIYKTPPIKYFGGFNFSQILPDPSKPSKRSPIEDLLFNKVTLA
DAGFIKQYGDCLGDIAARDLICAQKFKGLTVLPPLLTDEMIAQYTSALLAGTITSGWT
FGAGPALQIPFPMQMAYRFNGIGVTQNVLYENQKLIANQFNSAIGKIQDSLSSTPSAL
GKLQDVVNHNAQALNTLVKQLSSKFGAISSVLNDIFSRLDPPEAEVQIDRLITGRLQS
LQTYVTQQLIRAAEIRASANLAATKMSECVLGQSKRVDFCGKGYHLMSFPQSAPHG
VVFLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISGINA
SVVNIQKEIDRLNEVAKNLNESLIDLQELGKYEQYIKWP
Nucleotide Sequence of BA.1 S371 S373 S375 (Signal peptide in bold; GS linker is
in italics; TM and CT From F is in bold and double underlined; Spike ectodomain
is underlined; S371 S373 S375 is in bold, underlined and italics): SEQ ID NO: 30
ATGTTCGTGTTTCTGGTGCTGCTGCCTCTGGTGTCCAGCCAGTGTGTGAACCT
GACCACAAGAACCCAGCTGCCTCCAGCCTACACCAACAGCTTTACCAGAGGCGT
GTACTACCCCGACAAGGTGTTCAGATCCAGCGTGCTGCACTCTACCCAGGACCTG
TTCCTGCCTTTCTTCAGCAACGTGACCTGGTTCCACgtgATCTCCGGCACCAATGGC
ACCAAGAGATTCGACAACCCCGTGCTGCCCTTCAACGACGGGGTGTACTTTGCCA
GCatcGAGAAGTCCAACATCATCAGAGGCTGGATCTTCGGCACCACACTGGACAG
CAAGACCCAGAGCCTGCTGATCGTGAACAACGCCACCAACGTGGTCATCAAAGT
GTGCGAGTTCCAGTTCTGCAACGACCCCTTCCTGgacCACAAGAACAACAAGAGC
TGGATGGAAAGCGAGTTCCGGGTGTACAGCAGCGCCAACAACTGCACCTTCGAG
TACGTGTCCCAGCCTTTCCTGATGGACCTGGAAGGCAAGCAGGGCAACTTCAAG
AACCTGCGCGAGTTCGTGTTCAAGAACATCGACGGCTACTTCAAGATCTACAGCA
AGCACACCCCTATCatcGTGCGGgaacccgaaGATCTGCCTCAGGGCTTCTCTGCTCTG
GAACCCCTGGTGGATCTGCCCATCGGCATCAACATCACCCGGTTTCAGACACTGC
TGGCCCTGCACAGAAGCTACCTGACACCTGGCGATAGCAGCAGCGGATGGACAG
CTGGTGCCGCCGCTTACTATGTGGGCTACCTGCAGCCTAGAACCTTTCTGCTGAA
GTACAACGAGAACGGCACCATCACCGACGCCGTGGATTGTGCTCTGGATCCTCTG
AGCGAGACAAAGTGCACCCTGAAGTCCTTCACCGTGGAgAAGGGCATCTACCAG
ACCAGCAACTTCCGGGTGCAGCCCACCGAATCCATCGTGCGGTTCCCCAATATCA
CCAATCTGTGCCCCTTCgacGAGGTGTTCAATGCCACCAGATTCGCCTCTGTGTAC
GCCTGGAACCGGAAGCGGATCAGCAATTGCGTGGCCGACTACTCCGTGCTGTAC
AACtccGCCagcTTCagcACCTTCAAGTGCTACGGCGTGTCCCCTACCAAGCTGAACG
ACCTGTGCTTCACAAACGTGTACGCCGACAGCTTCGTGATCCGGGGAGATGAAG
TGCGGCAGATTGCCCCTGGACAGACAGGCaacATCGCCGACTACAACTACAAGCT
GCCCGACGACTTCACCGGCTGTGTGATTGCCTGGAACAGCAACaagCTGGACTCCA
AAGTCagcGGCAACTACAATTACCTGTACCGGCTGTTCCGGAAGTCCAATCTGAAG
CCCTTCGAGCGGGACATCTCCACCGAGATCTATCAGGCCGGCaacaagCCTTGTAAC
GGCGTGgccGGCTTCAACTGCTACTTCCCACTGagaTCCTACagcTTTagaCCCACAtacG
GCGTGGGCcacCAGCCCTACAGAGTGGTGGTGCTGAGCTTCGAACTGCTGCATGCC
CCTGCCACAGTGTGCGGCCCTAAGAAgAGCACCAATCTCGTGAAGAACAAATGC
GTGAACTTCAACTTCAACGGCCTGaagGGCACCGGCGTGCTGACAGAGAGCAACA
AGAAGTTCCTGCCATTCCAGCAGTTTGGCCGGGATATCGCCGATACCACAGACGC
CGTTAGAGATCCCCAGACACTGGAAATCCTGGACATCACCCCTTGCAGCTTCGGC
GGAGTGTCTGTGATCACCCCTGGCACCAACACCAGCAATCAGGTGGCAGTGCTG
TACCAGggcGTGAACTGTACCGAAGTGCCCGTGGCCATTCACGCCGATCAGCTGA
CACCTACATGGCGGGTGTACTCCACCGGCAGCAATGTGTTTCAGACCAGAGCCG
GCTGTCTGATCGGAGCCGAGtacGTGAACAATAGCTACGAGTGCGACATCCCCATC
GGCGCTGGCATCTGTGCCAGCTACCAGACACAGACAaagAGCcacGCCTCTGTGGC
CAGCCAGAGCATCATTGCCTACACAATGTCTCTGGGCGCCGAGAACAGCGTGGC
CTACTCCAACAACTCTATCGCTATCCCCACCAACTTCACCATCAGCGTGACCACA
GAGATCCTGCCTGTGTCCATGACCAAGACCAGCGTGGACTGCACCATGTACATCT
GCGGCGATTCCACCGAGTGCTCCAACCTGCTGCTGCAGTACGGCAGCTTCTGCAC
CCAGCTGaagAGAGCCCTGACAGGGATCGCCGTGGAACAGGACAAGAACACCCA
AGAGGTGTTCGCCCAAGTGAAGCAGATCTACAAGACCCCTCCTATCAAGtacTTCG
GCGGCTTCAATTTCAGCCAGATTCTGCCCGATCCTAGCAAGCCCAGCAAGCGGA
GCcctATCGAGGACCTGCTGTTCAACAAAGTGACACTGGCCGACGCCGGCTTCATC
AAGCAGTATGGCGATTGTCTGGGCGACATTGCCGCCAGGGATCTGATTTGCGCCC
AGAAGTTTaagGGACTGACAGTGCTGCCTCCTCTGCTGACCGATGAGATGATCGCC
CAGTACACATCTGCCCTGCTGGCCGGCACAATCACAAGCGGCTGGACATTTGGA
GCTGGCcctGCTCTGCAGATCCCCTTTccaATGCAGATGGCCTACCGGTTCAACGGC
ATCGGAGTGACCCAGAATGTGCTGTACGAGAACCAGAAGCTGATCGCCAACCAG
TTCAACAGCGCCATCGGCAAGATCCAGGACAGCCTGAGCAGCACAcccAGCGCCC
TGGGAAAGCTGCAGGACGTGGTCAACcacAATGCCCAGGCACTGAACACCCTGGT
CAAGCAGCTGTCCTCCaagTTCGGCGCCATCAGCTCTGTGCTGAACGATATCttcAG
CAGACTGGACccccctGAAGCCGAGGTGCAGATCGACAGACTGATCACCGGAAGGC
TGCAGTCCCTGCAGACCTACGTTACCCAGCAGCTGATCAGAGCCGCCGAGATTA
GAGCCTCTGCCAATCTGGCCGCCACCAAGATGTCTGAGTGTGTGCTGGGCCAGA
GCAAGAGAGTGGACTTTTGCGGCAAGGGCTACCACCTGATGAGCTTCCCTCAGTC
TGCCCCTCACGGCGTGGTGTTTCTGCACGTGACATACGTGCCCGCTCAAGAGAAG
AATTTCACCACCGCTCCAGCCATCTGCCACGACGGCAAAGCCCACTTTCCTAGAG
AAGGCGTGTTCGTGTCCAACGGCACCCATTGGTTCGTGACCCAGCGGAACTTCTA
CGAGCCCCAGATCATCACCACCGACAACACCTTCGTGTCTGGCAACTGCGACGTC
GTGATCGGCATTGTGAACAATACCGTGTACGACCCTCTGCAGCCCGAGCTGGAC
AGCTTCAAAGAGGAACTGGATAAGTACTTTAAGAACCACACAAGCCCCGACGTG
GACCTGGGCGATATCAGCGGAATCAATGCCAGCGTCGTGAACATCCAGAAAGAG
ATCGACCGGCTGAACGAGGTGGCCAAGAATCTGAACGAGAGCCTGATCGACCTG
CAAGAACTGGGGAAGTACGAGCAGTACATCAAGTGGCCCggcggaggtgggtcgCTCAT
AACATACATCGTCCTGACTATAATCAGCTTGGTATTTGGTATTTTGTCTTTGA
TTCTTGCATGCTATTTGATGTATAAACAGAAAGCTCAGCAGAAGACTCTCCT
GTGGCTCGGTAACAACACACTCGACCAGATGAGAGCAACTACAAAGATGTG
A
Amino Acid Sequence of BA.1 S371 S373 S375 (Signal peptide in bold; GS linker is
in italics; TM and CT From F is in bold and double underlined; Spike ectodomain
is underlined; S371 S373 S375 is in bold, underlined and italics): SEQ ID NO: 31
MFVFLVLLPLVSSQCVNLTTRTQLPPAYTNSFTRGVYYPDKVFRSSVLHSTQDLFLP
FFSNVTWFHVISGTNGTKRFDNPVLPFNDGVYFASIEKSNIIRGWIFGTTLDSKTQSLL
IVNNATNVVIKVCEFQFCNDPFLDHKNNKSWMESEFRVYSSANNCTFEYVSQPFLM
DLEGKQGNFKNLREFVFKNIDGYFKIYSKHTPIIVREPEDLPQGFSALEPLVDLPIGINI
TRFQTLLALHRSYLTPGDSSSGWTAGAAAYYVGYLQPRTFLLKYNENGTITDAVDC
ALDPLSETKCTLKSFTVEKGIYQTSNFRVQPTESIVRFPNITNLCPFDEVFNATRFASV
YAWNRKRISNCVADYSVLYNSASFSTFKCYGVSPTKLNDLCFTNVYADSFVIRGDEV
RQIAPGQTGNIADYNYKLPDDFTGCVIAWNSNKLDSKVSGNYNYLYRLFRKSNLKPF
ERDISTEIYQAGNKPCNGVAGFNCYFPLRSYSFRPTYGVGHQPYRVVVLSFELLHAP
ATVCGPKKSTNLVKNKCVNFNFNGLKGTGVLTESNKKFLPFQQFGRDIADTTDAVR
DPQTLEILDITPCSFGGVSVITPGTNTSNQVAVLYQGVNCTEVPVAIHADQLTPTWRV
YSTGSNVFQTRAGCLIGAEYVNNSYECDIPIGAGICASYQTQTKSHASVASQSIIAYT
MSLGAENSVAYSNNSIAIPTNFTISVTTEILPVSMTKTSVDCTMYICGDSTECSNLLLQ
YGSFCTQLKRALTGIAVEQDKNTQEVFAQVKQIYKTPPIKYFGGFNFSQILPDPSKPS
KRSPIEDLLFNKVTLADAGFIKQYGDCLGDIAARDLICAQKFKGLTVLPPLLTDEMIA
QYTSALLAGTITSGWTFGAGPALQIPFPMQMAYRENGIGVTQNVLYENQKLIANQFN
SAIGKIQDSLSSTPSALGKLQDVVNHNAQALNTLVKQLSSKFGAISSVLNDIFSRLDPP
EAEVQIDRLITGRLQSLQTYVTQQLIRAAEIRASANLAATKMSECVLGQSKRVDFCG
KGYHLMSFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKEELDKYFKN
HTSPDVDLGDISGINASVVNIQKEIDRLNEVAKNLNESLIDLQELGKYEQYIKWPGGG
GSLITYIVLTIISLVFGILSLILACYLMYKQKAQQKTLLWLGNNTLDQMRATTK
M*
Nucleotide Sequence of BA.1 S371 S373 S375 (Signal peptide in bold; Spike
ectodomain is underlined; S371 S373 S375 is in bold, underlined and italics):
SEQ ID NO: 32
ATGTTCGTGTTTCTGGTGCTGCTGCCTCTGGTGTCCAGCCAGTGTGTGAACCT
GACCACAAGAACCCAGCTGCCTCCAGCCTACACCAACAGCTTTACCAGAGGCGT
GTACTACCCCGACAAGGTGTTCAGATCCAGCGTGCTGCACTCTACCCAGGACCTG
TTCCTGCCTTTCTTCAGCAACGTGACCTGGTTCCACgtgATCTCCGGCACCAATGGC
ACCAAGAGATTCGACAACCCCGTGCTGCCCTTCAACGACGGGGTGTACTTTGCCA
GCatcGAGAAGTCCAACATCATCAGAGGCTGGATCTTCGGCACCACACTGGACAG
CAAGACCCAGAGCCTGCTGATCGTGAACAACGCCACCAACGTGGTCATCAAAGT
GTGCGAGTTCCAGTTCTGCAACGACCCCTTCCTGgacCACAAGAACAACAAGAGC
TGGATGGAAAGCGAGTTCCGGGTGTACAGCAGCGCCAACAACTGCACCTTCGAG
TACGTGTCCCAGCCTTTCCTGATGGACCTGGAAGGCAAGCAGGGCAACTTCAAG
AACCTGCGCGAGTTCGTGTTCAAGAACATCGACGGCTACTTCAAGATCTACAGCA
AGCACACCCCTATCatcGTGCGGgaacccgaaGATCTGCCTCAGGGCTTCTCTGCTCTG
GAACCCCTGGTGGATCTGCCCATCGGCATCAACATCACCCGGTTTCAGACACTGC
TGGCCCTGCACAGAAGCTACCTGACACCTGGCGATAGCAGCAGCGGATGGACAG
CTGGTGCCGCCGCTTACTATGTGGGCTACCTGCAGCCTAGAACCTTTCTGCTGAA
GTACAACGAGAACGGCACCATCACCGACGCCGTGGATTGTGCTCTGGATCCTCTG
AGCGAGACAAAGTGCACCCTGAAGTCCTTCACCGTGGAgAAGGGCATCTACCAG
ACCAGCAACTTCCGGGTGCAGCCCACCGAATCCATCGTGCGGTTCCCCAATATCA
CCAATCTGTGCCCCTTCgacGAGGTGTTCAATGCCACCAGATTCGCCTCTGTGTAC
GCCTGGAACCGGAAGCGGATCAGCAATTGCGTGGCCGACTACTCCGTGCTGTAC
AACtccGCCagcTTCagcACCTTCAAGTGCTACGGCGTGTCCCCTACCAAGCTGAACG
ACCTGTGCTTCACAAACGTGTACGCCGACAGCTTCGTGATCCGGGGAGATGAAG
TGCGGCAGATTGCCCCTGGACAGACAGGCaacATCGCCGACTACAACTACAAGCT
GCCCGACGACTTCACCGGCTGTGTGATTGCCTGGAACAGCAACaagCTGGACTCCA
AAGTCagcGGCAACTACAATTACCTGTACCGGCTGTTCCGGAAGTCCAATCTGAAG
CCCTTCGAGCGGGACATCTCCACCGAGATCTATCAGGCCGGCaacaagCCTTGTAAC
GGCGTGgccGGCTTCAACTGCTACTTCCCACTGagaTCCTACagcTTTagaCCCACAtacG
GCGTGGGCcacCAGCCCTACAGAGTGGTGGTGCTGAGCTTCGAACTGCTGCATGCC
CCTGCCACAGTGTGCGGCCCTAAGAAgAGCACCAATCTCGTGAAGAACAAATGC
GTGAACTTCAACTTCAACGGCCTGaagGGCACCGGCGTGCTGACAGAGAGCAACA
AGAAGTTCCTGCCATTCCAGCAGTTTGGCCGGGATATCGCCGATACCACAGACGC
CGTTAGAGATCCCCAGACACTGGAAATCCTGGACATCACCCCTTGCAGCTTCGGC
GGAGTGTCTGTGATCACCCCTGGCACCAACACCAGCAATCAGGTGGCAGTGCTG
TACCAGggcGTGAACTGTACCGAAGTGCCCGTGGCCATTCACGCCGATCAGCTGA
CACCTACATGGCGGGTGTACTCCACCGGCAGCAATGTGTTTCAGACCAGAGCCG
GCTGTCTGATCGGAGCCGAGtacGTGAACAATAGCTACGAGTGCGACATCCCCATC
GGCGCTGGCATCTGTGCCAGCTACCAGACACAGACAaagAGCcacGCCTCTGTGGC
CAGCCAGAGCATCATTGCCTACACAATGTCTCTGGGCGCCGAGAACAGCGTGGC
CTACTCCAACAACTCTATCGCTATCCCCACCAACTTCACCATCAGCGTGACCACA
GAGATCCTGCCTGTGTCCATGACCAAGACCAGCGTGGACTGCACCATGTACATCT
GCGGCGATTCCACCGAGTGCTCCAACCTGCTGCTGCAGTACGGCAGCTTCTGCAC
CCAGCTGaagAGAGCCCTGACAGGGATCGCCGTGGAACAGGACAAGAACACCCA
AGAGGTGTTCGCCCAAGTGAAGCAGATCTACAAGACCCCTCCTATCAAGtacTTCG
GCGGCTTCAATTTCAGCCAGATTCTGCCCGATCCTAGCAAGCCCAGCAAGCGGA
GCcctATCGAGGACCTGCTGTTCAACAAAGTGACACTGGCCGACGCCGGCTTCATC
AAGCAGTATGGCGATTGTCTGGGCGACATTGCCGCCAGGGATCTGATTTGCGCCC
AGAAGTTTaagGGACTGACAGTGCTGCCTCCTCTGCTGACCGATGAGATGATCGCC
CAGTACACATCTGCCCTGCTGGCCGGCACAATCACAAGCGGCTGGACATTTGGA
GCTGGCcctGCTCTGCAGATCCCCTTTccaATGCAGATGGCCTACCGGTTCAACGGC
ATCGGAGTGACCCAGAATGTGCTGTACGAGAACCAGAAGCTGATCGCCAACCAG
TTCAACAGCGCCATCGGCAAGATCCAGGACAGCCTGAGCAGCACAcccAGCGCCC
TGGGAAAGCTGCAGGACGTGGTCAACcacAATGCCCAGGCACTGAACACCCTGGT
CAAGCAGCTGTCCTCCaagTTCGGCGCCATCAGCTCTGTGCTGAACGATATCttcAG
CAGACTGGACccccctGAAGCCGAGGTGCAGATCGACAGACTGATCACCGGAAGGC
TGCAGTCCCTGCAGACCTACGTTACCCAGCAGCTGATCAGAGCCGCCGAGATTA
GAGCCTCTGCCAATCTGGCCGCCACCAAGATGTCTGAGTGTGTGCTGGGCCAGA
GCAAGAGAGTGGACTTTTGCGGCAAGGGCTACCACCTGATGAGCTTCCCTCAGTC
TGCCCCTCACGGCGTGGTGTTTCTGCACGTGACATACGTGCCCGCTCAAGAGAAG
AATTTCACCACCGCTCCAGCCATCTGCCACGACGGCAAAGCCCACTTTCCTAGAG
AAGGCGTGTTCGTGTCCAACGGCACCCATTGGTTCGTGACCCAGCGGAACTTCTA
CGAGCCCCAGATCATCACCACCGACAACACCTTCGTGTCTGGCAACTGCGACGTC
GTGATCGGCATTGTGAACAATACCGTGTACGACCCTCTGCAGCCCGAGCTGGAC
AGCTTCAAAGAGGAACTGGATAAGTACTTTAAGAACCACACAAGCCCCGACGTG
GACCTGGGCGATATCAGCGGAATCAATGCCAGCGTCGTGAACATCCAGAAAGAG
ATCGACCGGCTGAACGAGGTGGCCAAGAATCTGAACGAGAGCCTGATCGACCTG
CAAGAACTGGGGAAGTACGAGCAGTACATCAAGTGGCCC
Amino Acid Sequence of BA.1 S371 S373 S375 (Signal peptide in bold; Spike
ectodomain is underlined; S371 S373 S375 is in bold, underlined and italics): SEQ
ID NO: 33
MFVFLVLLPLVSSQCVNLTTRTQLPPAYTNSFTRGVYYPDKVFRSSVLHSTQDLFLP
FFSNVTWFHVISGTNGTKRFDNPVLPFNDGVYFASIEKSNIIRGWIFGTTLDSKTQSLL
IVNNATNVVIKVCEFQFCNDPFLDHKNNKSWMESEFRVYSSANNCTFEYVSQPFLM
DLEGKQGNFKNLREFVFKNIDGYFKIYSKHTPIIVREPEDLPQGFSALEPLVDLPIGINI
TRFQTLLALHRSYLTPGDSSSGWTAGAAAYYVGYLQPRTFLLKYNENGTITDAVDC
ALDPLSETKCTLKSFTVEKGIYQTSNFRVQPTESIVRFPNITNLCPFDEVFNATRFASV
YAWNRKRISNCVADYSVLYNSASFSTFKCYGVSPTKLNDLCFTNVYADSFVIRGDEV
RQIAPGQTGNIADYNYKLPDDFTGCVIAWNSNKLDSKVSGNYNYLYRLFRKSNLKPF
ERDISTEIYQAGNKPCNGVAGFNCYFPLRSYSFRPTYGVGHQPYRVVVLSFELLHAP
ATVCGPKKSTNLVKNKCVNFNFNGLKGTGVLTESNKKFLPFQQFGRDIADTTDAVR
DPQTLEILDITPCSFGGVSVITPGTNTSNQVAVLYQGVNCTEVPVAIHADQLTPTWRV
YSTGSNVFQTRAGCLIGAEYVNNSYECDIPIGAGICASYQTQTKSHASVASQSIIAYT
MSLGAENSVAYSNNSIAIPTNFTISVTTEILPVSMTKTSVDCTMYICGDSTECSNLLLQ
YGSFCTQLKRALTGIAVEQDKNTQEVFAQVKQIYKTPPIKYFGGFNFSQILPDPSKPS
KRSPIEDLLFNKVTLADAGFIKQYGDCLGDIAARDLICAQKFKGLTVLPPLLTDEMIA
QYTSALLAGTITSGWTFGAGPALQIPFPMQMAYRENGIGVTQNVLYENQKLIANQFN
SAIGKIQDSLSSTPSALGKLQDVVNHNAQALNTLVKQLSSKFGAISSVLNDIFSRLDPP
EAEVQIDRLITGRLQSLQTYVTQQLIRAAEIRASANLAATKMSECVLGQSKRVDFCG
KGYHLMSFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKEELDKYFKN
HTSPDVDLGDISGINASVVNIQKEIDRLNEVAKNLNESLIDLQELGKYEQYIKWP
Nucleotide Sequence of BA.1 S371 S373 S375 (Spike ectodomain without signal
peptide is underlined; S371 S373 S375 is in bold, underlined and italics): SEQ ID
NO: 34
CAGTGTGTGAACCTGACCACAAGAACCCAGCTGCCTCCAGCCTACACCAACAGC
TTTACCAGAGGCGTGTACTACCCCGACAAGGTGTTCAGATCCAGCGTGCTGCACT
CTACCCAGGACCTGTTCCTGCCTTTCTTCAGCAACGTGACCTGGTTCCACgtgATCT
CCGGCACCAATGGCACCAAGAGATTCGACAACCCCGTGCTGCCCTTCAACGACG
GGGTGTACTTTGCCAGCatcGAGAAGTCCAACATCATCAGAGGCTGGATCTTCGGC
ACCACACTGGACAGCAAGACCCAGAGCCTGCTGATCGTGAACAACGCCACCAAC
GTGGTCATCAAAGTGTGCGAGTTCCAGTTCTGCAACGACCCCTTCCTGgacCACAA
GAACAACAAGAGCTGGATGGAAAGCGAGTTCCGGGTGTACAGCAGCGCCAACA
ACTGCACCTTCGAGTACGTGTCCCAGCCTTTCCTGATGGACCTGGAAGGCAAGCA
GGGCAACTTCAAGAACCTGCGCGAGTTCGTGTTCAAGAACATCGACGGCTACTTC
AAGATCTACAGCAAGCACACCCCTATCatcGTGCGGgaacccgaaGATCTGCCTCAGGG
CTTCTCTGCTCTGGAACCCCTGGTGGATCTGCCCATCGGCATCAACATCACCCGG
TTTCAGACACTGCTGGCCCTGCACAGAAGCTACCTGACACCTGGCGATAGCAGC
AGCGGATGGACAGCTGGTGCCGCCGCTTACTATGTGGGCTACCTGCAGCCTAGA
ACCTTTCTGCTGAAGTACAACGAGAACGGCACCATCACCGACGCCGTGGATTGT
GCTCTGGATCCTCTGAGCGAGACAAAGTGCACCCTGAAGTCCTTCACCGTGGAgA
AGGGCATCTACCAGACCAGCAACTTCCGGGTGCAGCCCACCGAATCCATCGTGC
GGTTCCCCAATATCACCAATCTGTGCCCCTTCgacGAGGTGTTCAATGCCACCAGA
TTCGCCTCTGTGTACGCCTGGAACCGGAAGCGGATCAGCAATTGCGTGGCCGACT
ACTCCGTGCTGTACAACtccGCCagcTTCagcACCTTCAAGTGCTACGGCGTGTCCCC
TACCAAGCTGAACGACCTGTGCTTCACAAACGTGTACGCCGACAGCTTCGTGATC
CGGGGAGATGAAGTGCGGCAGATTGCCCCTGGACAGACAGGCaacATCGCCGACT
ACAACTACAAGCTGCCCGACGACTTCACCGGCTGTGTGATTGCCTGGAACAGCA
ACaagCTGGACTCCAAAGTCagcGGCAACTACAATTACCTGTACCGGCTGTTCCGGA
AGTCCAATCTGAAGCCCTTCGAGCGGGACATCTCCACCGAGATCTATCAGGCCG
GCaacaagCCTTGTAACGGCGTGgccGGCTTCAACTGCTACTTCCCACTGagaTCCTACa
gcTTTagaCCCACAtacGGCGTGGGCcacCAGCCCTACAGAGTGGTGGTGCTGAGCTTC
GAACTGCTGCATGCCCCTGCCACAGTGTGCGGCCCTAAGAAgAGCACCAATCTCG
TGAAGAACAAATGCGTGAACTTCAACTTCAACGGCCTGaagGGCACCGGCGTGCT
GACAGAGAGCAACAAGAAGTTCCTGCCATTCCAGCAGTTTGGCCGGGATATCGC
CGATACCACAGACGCCGTTAGAGATCCCCAGACACTGGAAATCCTGGACATCAC
CCCTTGCAGCTTCGGCGGAGTGTCTGTGATCACCCCTGGCACCAACACCAGCAAT
CAGGTGGCAGTGCTGTACCAGggcGTGAACTGTACCGAAGTGCCCGTGGCCATTC
ACGCCGATCAGCTGACACCTACATGGCGGGTGTACTCCACCGGCAGCAATGTGTT
TCAGACCAGAGCCGGCTGTCTGATCGGAGCCGAGtacGTGAACAATAGCTACGAG
TGCGACATCCCCATCGGCGCTGGCATCTGTGCCAGCTACCAGACACAGACAaagA
GCcacGCCTCTGTGGCCAGCCAGAGCATCATTGCCTACACAATGTCTCTGGGCGCC
GAGAACAGCGTGGCCTACTCCAACAACTCTATCGCTATCCCCACCAACTTCACCA
TCAGCGTGACCACAGAGATCCTGCCTGTGTCCATGACCAAGACCAGCGTGGACT
GCACCATGTACATCTGCGGCGATTCCACCGAGTGCTCCAACCTGCTGCTGCAGTA
CGGCAGCTTCTGCACCCAGCTGaagAGAGCCCTGACAGGGATCGCCGTGGAACAG
GACAAGAACACCCAAGAGGTGTTCGCCCAAGTGAAGCAGATCTACAAGACCCCT
CCTATCAAGtacTTCGGCGGCTTCAATTTCAGCCAGATTCTGCCCGATCCTAGCAA
GCCCAGCAAGCGGAGCcctATCGAGGACCTGCTGTTCAACAAAGTGACACTGGCC
GACGCCGGCTTCATCAAGCAGTATGGCGATTGTCTGGGCGACATTGCCGCCAGG
GATCTGATTTGCGCCCAGAAGTTTaagGGACTGACAGTGCTGCCTCCTCTGCTGAC
CGATGAGATGATCGCCCAGTACACATCTGCCCTGCTGGCCGGCACAATCACAAG
CGGCTGGACATTTGGAGCTGGCcctGCTCTGCAGATCCCCTTTccaATGCAGATGGC
CTACCGGTTCAACGGCATCGGAGTGACCCAGAATGTGCTGTACGAGAACCAGAA
GCTGATCGCCAACCAGTTCAACAGCGCCATCGGCAAGATCCAGGACAGCCTGAG
CAGCACAcccAGCGCCCTGGGAAAGCTGCAGGACGTGGTCAACcacAATGCCCAGG
CACTGAACACCCTGGTCAAGCAGCTGTCCTCCaagTTCGGCGCCATCAGCTCTGTG
CTGAACGATATCttcAGCAGACTGGACccccctGAAGCCGAGGTGCAGATCGACAGAC
TGATCACCGGAAGGCTGCAGTCCCTGCAGACCTACGTTACCCAGCAGCTGATCA
GAGCCGCCGAGATTAGAGCCTCTGCCAATCTGGCCGCCACCAAGATGTCTGAGT
GTGTGCTGGGCCAGAGCAAGAGAGTGGACTTTTGCGGCAAGGGCTACCACCTGA
TGAGCTTCCCTCAGTCTGCCCCTCACGGCGTGGTGTTTCTGCACGTGACATACGT
GCCCGCTCAAGAGAAGAATTTCACCACCGCTCCAGCCATCTGCCACGACGGCAA
AGCCCACTTTCCTAGAGAAGGCGTGTTCGTGTCCAACGGCACCCATTGGTTCGTG
ACCCAGCGGAACTTCTACGAGCCCCAGATCATCACCACCGACAACACCTTCGTGT
CTGGCAACTGCGACGTCGTGATCGGCATTGTGAACAATACCGTGTACGACCCTCT
GCAGCCCGAGCTGGACAGCTTCAAAGAGGAACTGGATAAGTACTTTAAGAACCA
CACAAGCCCCGACGTGGACCTGGGCGATATCAGCGGAATCAATGCCAGCGTCGT
GAACATCCAGAAAGAGATCGACCGGCTGAACGAGGTGGCCAAGAATCTGAACG
AGAGCCTGATCGACCTGCAAGAACTGGGGAAGTACGAGCAGTACATCAAGTGGC
CC
Amino Acid Sequence of BA.1 S371 S373 S375 (Spike ectodomain without signal
peptide is underlined; S371 S373 S375 is in bold, underlined and italics): SEQ ID
NO: 35
QCVNLTTRTQLPPAYTNSFTRGVYYPDKVFRSSVLHSTQDLFLPFFSNVTWFHVISGT
NGTKRFDNPVLPFNDGVYFASIEKSNIIRGWIFGTTLDSKTQSLLIVNNATNVVIKVCE
FQFCNDPFLDHKNNKSWMESEFRVYSSANNCTFEYVSQPFLMDLEGKQGNFKNLRE
FVFKNIDGYFKIYSKHTPIIVREPEDLPQGFSALEPLVDLPIGINITRFQTLLALHRSYLT
PGDSSSGWTAGAAAYYVGYLQPRTFLLKYNENGTITDAVDCALDPLSETKCTLKSFT
VEKGIYQTSNFRVQPTESIVRFPNITNLCPFDEVFNATRFASVYAWNRKRISNCVADY
SVLYNSASFSTFKCYGVSPTKLNDLCFTNVYADSFVIRGDEVRQIAPGQTGNIADYN
YKLPDDFTGCVIAWNSNKLDSKVSGNYNYLYRLFRKSNLKPFERDISTEIYQAGNKP
CNGVAGFNCYFPLRSYSFRPTYGVGHQPYRVVVLSFELLHAPATVCGPKKSTNLVK
NKCVNFNFNGLKGTGVLTESNKKFLPFQQFGRDIADTTDAVRDPQTLEILDITPCSFG
GVSVITPGTNTSNQVAVLYQGVNCTEVPVAIHADQLTPTWRVYSTGSNVFQTRAGC
LIGAEYVNNSYECDIPIGAGICASYQTQTKSHASVASQSIIAYTMSLGAENSVAYSNN
SIAIPTNFTISVTTEILPVSMTKTSVDCTMYICGDSTECSNLLLQYGSFCTQLKRALTGI
AVEQDKNTQEVFAQVKQIYKTPPIKYFGGFNFSQILPDPSKPSKRSPIEDLLFNKVTLA
DAGFIKQYGDCLGDIAARDLICAQKFKGLTVLPPLLTDEMIAQYTSALLAGTITSGWT
FGAGPALQIPFPMQMAYRENGIGVTQNVLYENQKLIANQFNSAIGKIQDSLSSTPSAL
GKLQDVVNHNAQALNTLVKQLSSKFGAISSVLNDIFSRLDPPEAEVQIDRLITGRLQS
LQTYVTQQLIRAAEIRASANLAATKMSECVLGQSKRVDFCGKGYHLMSFPQSAPHG
VVFLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISGINA
SVVNIQKEIDRLNEVAKNLNESLIDLQELGKYEQYIKWP
Nucleotide Sequence of BA.2 (Signal peptide is in bold and underlined; GS linker
is in italics; TM and CT From F is in bold and double underlined; Spike ectodomain
is underlined): SEQ ID NO: 36
ATGTTCGTGTTTCTGGTGCTGCTGCCTCTGGTGTCCAGCCAGTGTGTGAACCT
GatcACAAGAACCCAGagcTACACCAACAGCTTTACCAGAGGCGTGTACTACCCCG
ACAAGGTGTTCAGATCCAGCGTGCTGCACTCTACCCAGGACCTGTTCCTGCCTTT
CTTCAGCAACGTGACCTGGTTCCACGCCATCCACGTGTCCGGCACCAATGGCACC
AAGAGATTCGACAACCCCGTGCTGCCCTTCAACGACGGGGTGTACTTTGCCAGCA
CCGAGAAGTCCAACATCATCAGAGGCTGGATCTTCGGCACCACACTGGACAGCA
AGACCCAGAGCCTGCTGATCGTGAACAACGCCACCAACGTGGTCATCAAAGTGT
GCGAGTTCCAGTTCTGCAACGACCCCTTCCTGgacGTCTACTATCACAAGAACAAC
AAGAGCTGGATGGAAAGCGAGTTCCGGGTGTACAGCAGCGCCAACAACTGCACC
TTCGAGTACGTGTCCCAGCCTTTCCTGATGGACCTGGAAGGCAAGCAGGGCAACT
TCAAGAACCTGCGCGAGTTCGTGTTCAAGAACATCGACGGCTACTTCAAGATCTA
CAGCAAGCACACCCCTATCAACCTCggcCGGGATCTGCCTCAGGGCTTCTCTGCTC
TGGAACCCCTGGTGGATCTGCCCATCGGCATCAACATCACCCGGTTTCAGACACT
GCTGGCCCTGCACAGAAGCTACCTGACACCTGGCGATAGCAGCAGCGGATGGAC
AGCTGGTGCCGCCGCTTACTATGTGGGCTACCTGCAGCCTAGAACCTTTCTGCTG
AAGTACAACGAGAACGGCACCATCACCGACGCCGTGGATTGTGCTCTGGATCCT
CTGAGCGAGACAAAGTGCACCCTGAAGTCCTTCACCGTGGAgAAGGGCATCTAC
CAGACCAGCAACTTCCGGGTGCAGCCCACCGAATCCATCGTGCGGTTCCCCAATA
TCACCAATCTGTGCCCCTTCgacGAGGTGTTCAATGCCACCAGATTCGCCTCTGTG
TACGCCTGGAACCGGAAGCGGATCAGCAATTGCGTGGCCGACTACTCCGTGCTG
TACAACttcGCCcctTTCttcgccTTCAAGTGCTACGGCGTGTCCCCTACCAAGCTGAAC
GACCTGTGCTTCACAAACGTGTACGCCGACAGCTTCGTGATCCGGGGAaacGAAG
TGagcCAGATTGCCCCTGGACAGACAGGCaacATCGCCGACTACAACTACAAGCTG
CCCGACGACTTCACCGGCTGTGTGATTGCCTGGAACAGCAACaagCTGGACTCCAA
AGTCGGCGGCAACTACAATTACCTGTACCGGCTGTTCCGGAAGTCCAATCTGAAG
CCCTTCGAGCGGGACATCTCCACCGAGATCTATCAGGCCGGCaacaagCCTTGTAAC
GGCGTGgccGGCTTCAACTGCTACTTCCCACTGcggTCCTACGGCTTTcggCCCACAta
cGGCGTGGGCcacCAGCCCTACAGAGTGGTGGTGCTGAGCTTCGAACTGCTGCATG
CCCCTGCCACAGTGTGCGGCCCTAAGAAgAGCACCAATCTCGTGAAGAACAAAT
GCGTGAACTTCAACTTCAACGGCCTGACCGGCACCGGCGTGCTGACAGAGAGCA
ACAAGAAGTTCCTGCCATTCCAGCAGTTTGGCCGGGATATCGCCGATACCACAG
ACGCCGTTAGAGATCCCCAGACACTGGAAATCCTGGACATCACCCCTTGCAGCTT
CGGCGGAGTGTCTGTGATCACCCCTGGCACCAACACCAGCAATCAGGTGGCAGT
GCTGTACCAGggcGTGAACTGTACCGAAGTGCCCGTGGCCATTCACGCCGATCAG
CTGACACCTACATGGCGGGTGTACTCCACCGGCAGCAATGTGTTTCAGACCAGA
GCCGGCTGTCTGATCGGAGCCGAGtacGTGAACAATAGCTACGAGTGCGACATCC
CCATCGGCGCTGGCATCTGTGCCAGCTACCAGACACAGACAaagAGCcacGCCTCT
GTGGCCAGCCAGAGCATCATTGCCTACACAATGTCTCTGGGCGCCGAGAACAGC
GTGGCCTACTCCAACAACTCTATCGCTATCCCCACCAACTTCACCATCAGCGTGA
CCACAGAGATCCTGCCTGTGTCCATGACCAAGACCAGCGTGGACTGCACCATGT
ACATCTGCGGCGATTCCACCGAGTGCTCCAACCTGCTGCTGCAGTACGGCAGCTT
CTGCACCCAGCTGaagAGAGCCCTGACAGGGATCGCCGTGGAACAGGACAAGAAC
ACCCAAGAGGTGTTCGCCCAAGTGAAGCAGATCTACAAGACCCCTCCTATCAAGt
acTTCGGCGGCTTCAATTTCAGCCAGATTCTGCCCGATCCTAGCAAGCCCAGCAAG
CGGAGCcctATCGAGGACCTGCTGTTCAACAAAGTGACACTGGCCGACGCCGGCTT
CATCAAGCAGTATGGCGATTGTCTGGGCGACATTGCCGCCAGGGATCTGATTTGC
GCCCAGAAGTTTAACGGACTGACAGTGCTGCCTCCTCTGCTGACCGATGAGATGA
TCGCCCAGTACACATCTGCCCTGCTGGCCGGCACAATCACAAGCGGCTGGACATT
TGGAGCTGGCcctGCTCTGCAGATCCCCTTTccaATGCAGATGGCCTACCGGTTCAA
CGGCATCGGAGTGACCCAGAATGTGCTGTACGAGAACCAGAAGCTGATCGCCAA
CCAGTTCAACAGCGCCATCGGCAAGATCCAGGACAGCCTGAGCAGCACAcccAGC
GCCCTGGGAAAGCTGCAGGACGTGGTCAACcacAATGCCCAGGCACTGAACACCC
TGGTCAAGCAGCTGTCCTCCaagTTCGGCGCCATCAGCTCTGTGCTGAACGATATC
CTGAGCAGACTGGACccccctGAAGCCGAGGTGCAGATCGACAGACTGATCACCGG
AAGGCTGCAGTCCCTGCAGACCTACGTTACCCAGCAGCTGATCAGAGCCGCCGA
GATTAGAGCCTCTGCCAATCTGGCCGCCACCAAGATGTCTGAGTGTGTGCTGGGC
CAGAGCAAGAGAGTGGACTTTTGCGGCAAGGGCTACCACCTGATGAGCTTCCCT
CAGTCTGCCCCTCACGGCGTGGTGTTTCTGCACGTGACATACGTGCCCGCTCAAG
AGAAGAATTTCACCACCGCTCCAGCCATCTGCCACGACGGCAAAGCCCACTTTCC
TAGAGAAGGCGTGTTCGTGTCCAACGGCACCCATTGGTTCGTGACCCAGCGGAA
CTTCTACGAGCCCCAGATCATCACCACCGACAACACCTTCGTGTCTGGCAACTGC
GACGTCGTGATCGGCATTGTGAACAATACCGTGTACGACCCTCTGCAGCCCGAGC
TGGACAGCTTCAAAGAGGAACTGGATAAGTACTTTAAGAACCACACAAGCCCCG
ACGTGGACCTGGGCGATATCAGCGGAATCAATGCCAGCGTCGTGAACATCCAGA
AAGAGATCGACCGGCTGAACGAGGTGGCCAAGAATCTGAACGAGAGCCTGATCG
ACCTGCAAGAACTGGGGAAGTACGAGCAGTACATCAAGTGGCCCggcggaggtgggtcg
CTCATAACATACATCGTCCTGACTATAATCAGCTTGGTATTTGGTATTTTGTC
TTTGATTCTTGCATGCTATTTGATGTATAAACAGAAAGCTCAGCAGAAGACT
CTCCTGTGGCTCGGTAACAACACACTCGACCAGATGAGAGCAACTACAAAG
ATGTGA
Amino Acid Sequence of BA.2 (Signal peptide is in bold and underlined; GS linker
is in italics; TM and CT From F is in bold and double underlined; Spike ectodomain
is underlined): SEQ ID NO: 37
MFVFLVLLPLVSSQCVNLITRTQSYTNSFTRGVYYPDKVFRSSVLHSTQDLFLPFFSN
VTWFHAIHVSGTNGTKRFDNPVLPFNDGVYFASTEKSNIIRGWIFGTTLDSKTQSLLI
VNNATNVVIKVCEFQFCNDPFLDVYYHKNNKSWMESEFRVYSSANNCTFEYVSQPF
LMDLEGKQGNFKNLREFVFKNIDGYFKIYSKHTPINLGRDLPQGFSALEPLVDLPIGIN
ITRFQTLLALHRSYLTPGDSSSGWTAGAAAYYVGYLQPRTFLLKYNENGTITDAVDC
ALDPLSETKCTLKSFTVEKGIYQTSNFRVQPTESIVRFPNITNLCPFDEVFNATRFASV
YAWNRKRISNCVADYSVLYNFAPFFAFKCYGVSPTKLNDLCFTNVYADSFVIRGNE
VSQIAPGQTGNIADYNYKLPDDFTGCVIAWNSNKLDSKVGGNYNYLYRLFRKSNLK
PFERDISTEIYQAGNKPCNGVAGFNCYFPLRSYGFRPTYGVGHQPYRVVVLSFELLH
APATVCGPKKSTNLVKNKCVNFNFNGLTGTGVLTESNKKFLPFQQFGRDIADTTDA
VRDPQTLEILDITPCSFGGVSVITPGTNTSNQVAVLYQGVNCTEVPVAIHADQLTPTW
RVYSTGSNVFQTRAGCLIGAEYVNNSYECDIPIGAGICASYQTQTKSHASVASQSIIAY
TMSLGAENSVAYSNNSIAIPTNFTISVTTEILPVSMTKTSVDCTMYICGDSTECSNLLL
QYGSFCTQLKRALTGIAVEQDKNTQEVFAQVKQIYKTPPIKYFGGFNFSQILPDPSKP
SKRSPIEDLLFNKVTLADAGFIKQYGDCLGDIAARDLICAQKFNGLTVLPPLLTDEMI
AQYTSALLAGTITSGWTFGAGPALQIPFPMQMAYRENGIGVTQNVLYENQKLIANQF
NSAIGKIQDSLSSTPSALGKLQDVVNHNAQALNTLVKQLSSKFGAISSVLNDILSRLD
PPEAEVQIDRLITGRLQSLQTYVTQQLIRAAEIRASANLAATKMSECVLGQSKRVDFC
GKGYHLMSFPQSAPHGVVFLHVTYVPAQEKNFTTAPAICHDGKAHFPREGVFVSNG
THWFVTQRNFYEPQIITTDNTFVSGNCDVVIGIVNNTVYDPLQPELDSFKEELDKYFK
NHTSPDVDLGDISGINASVVNIQKEIDRLNEVAKNLNESLIDLQELGKYEQYIKWPGG
GGSLITYIVLTIISLVFGILSLILACYLMYKQKAQQKTLLWLGNNTLDQMRATTK
M*
Nucleotide Sequence of BA.2 (Signal peptide is in bold and underlined; Spike
ectodomain is underlined): SEQ ID NO: 38
ATGTTCGTGTTTCTGGTGCTGCTGCCTCTGGTGTCCAGCCAGTGTGTGAACCT
GatcACAAGAACCCAGagcTACACCAACAGCTTTACCAGAGGCGTGTACTACCCCG
ACAAGGTGTTCAGATCCAGCGTGCTGCACTCTACCCAGGACCTGTTCCTGCCTTT
CTTCAGCAACGTGACCTGGTTCCACGCCATCCACGTGTCCGGCACCAATGGCACC
AAGAGATTCGACAACCCCGTGCTGCCCTTCAACGACGGGGTGTACTTTGCCAGCA
CCGAGAAGTCCAACATCATCAGAGGCTGGATCTTCGGCACCACACTGGACAGCA
AGACCCAGAGCCTGCTGATCGTGAACAACGCCACCAACGTGGTCATCAAAGTGT
GCGAGTTCCAGTTCTGCAACGACCCCTTCCTGgacGTCTACTATCACAAGAACAAC
AAGAGCTGGATGGAAAGCGAGTTCCGGGTGTACAGCAGCGCCAACAACTGCACC
TTCGAGTACGTGTCCCAGCCTTTCCTGATGGACCTGGAAGGCAAGCAGGGCAACT
TCAAGAACCTGCGCGAGTTCGTGTTCAAGAACATCGACGGCTACTTCAAGATCTA
CAGCAAGCACACCCCTATCAACCTCggcCGGGATCTGCCTCAGGGCTTCTCTGCTC
TGGAACCCCTGGTGGATCTGCCCATCGGCATCAACATCACCCGGTTTCAGACACT
GCTGGCCCTGCACAGAAGCTACCTGACACCTGGCGATAGCAGCAGCGGATGGAC
AGCTGGTGCCGCCGCTTACTATGTGGGCTACCTGCAGCCTAGAACCTTTCTGCTG
AAGTACAACGAGAACGGCACCATCACCGACGCCGTGGATTGTGCTCTGGATCCT
CTGAGCGAGACAAAGTGCACCCTGAAGTCCTTCACCGTGGAgAAGGGCATCTAC
CAGACCAGCAACTTCCGGGTGCAGCCCACCGAATCCATCGTGCGGTTCCCCAATA
TCACCAATCTGTGCCCCTTCgacGAGGTGTTCAATGCCACCAGATTCGCCTCTGTG
TACGCCTGGAACCGGAAGCGGATCAGCAATTGCGTGGCCGACTACTCCGTGCTG
TACAACttcGCCcctTTCttcgccTTCAAGTGCTACGGCGTGTCCCCTACCAAGCTGAAC
GACCTGTGCTTCACAAACGTGTACGCCGACAGCTTCGTGATCCGGGGAaacGAAG
TGagcCAGATTGCCCCTGGACAGACAGGCaacATCGCCGACTACAACTACAAGCTG
CCCGACGACTTCACCGGCTGTGTGATTGCCTGGAACAGCAACaagCTGGACTCCAA
AGTCGGCGGCAACTACAATTACCTGTACCGGCTGTTCCGGAAGTCCAATCTGAAG
CCCTTCGAGCGGGACATCTCCACCGAGATCTATCAGGCCGGCaacaagCCTTGTAAC
GGCGTGgccGGCTTCAACTGCTACTTCCCACTGcggTCCTACGGCTTTcggCCCACAta
cGGCGTGGGCcacCAGCCCTACAGAGTGGTGGTGCTGAGCTTCGAACTGCTGCATG
CCCCTGCCACAGTGTGCGGCCCTAAGAAgAGCACCAATCTCGTGAAGAACAAAT
GCGTGAACTTCAACTTCAACGGCCTGACCGGCACCGGCGTGCTGACAGAGAGCA
ACAAGAAGTTCCTGCCATTCCAGCAGTTTGGCCGGGATATCGCCGATACCACAG
ACGCCGTTAGAGATCCCCAGACACTGGAAATCCTGGACATCACCCCTTGCAGCTT
CGGCGGAGTGTCTGTGATCACCCCTGGCACCAACACCAGCAATCAGGTGGCAGT
GCTGTACCAGggcGTGAACTGTACCGAAGTGCCCGTGGCCATTCACGCCGATCAG
CTGACACCTACATGGCGGGTGTACTCCACCGGCAGCAATGTGTTTCAGACCAGA
GCCGGCTGTCTGATCGGAGCCGAGtacGTGAACAATAGCTACGAGTGCGACATCC
CCATCGGCGCTGGCATCTGTGCCAGCTACCAGACACAGACAaagAGCcacGCCTCT
GTGGCCAGCCAGAGCATCATTGCCTACACAATGTCTCTGGGCGCCGAGAACAGC
GTGGCCTACTCCAACAACTCTATCGCTATCCCCACCAACTTCACCATCAGCGTGA
CCACAGAGATCCTGCCTGTGTCCATGACCAAGACCAGCGTGGACTGCACCATGT
ACATCTGCGGCGATTCCACCGAGTGCTCCAACCTGCTGCTGCAGTACGGCAGCTT
CTGCACCCAGCTGaagAGAGCCCTGACAGGGATCGCCGTGGAACAGGACAAGAAC
ACCCAAGAGGTGTTCGCCCAAGTGAAGCAGATCTACAAGACCCCTCCTATCAAGt
acTTCGGCGGCTTCAATTTCAGCCAGATTCTGCCCGATCCTAGCAAGCCCAGCAAG
CGGAGCcctATCGAGGACCTGCTGTTCAACAAAGTGACACTGGCCGACGCCGGCTT
CATCAAGCAGTATGGCGATTGTCTGGGCGACATTGCCGCCAGGGATCTGATTTGC
GCCCAGAAGTTTAACGGACTGACAGTGCTGCCTCCTCTGCTGACCGATGAGATGA
TCGCCCAGTACACATCTGCCCTGCTGGCCGGCACAATCACAAGCGGCTGGACATT
TGGAGCTGGCcctGCTCTGCAGATCCCCTTTccaATGCAGATGGCCTACCGGTTCAA
CGGCATCGGAGTGACCCAGAATGTGCTGTACGAGAACCAGAAGCTGATCGCCAA
CCAGTTCAACAGCGCCATCGGCAAGATCCAGGACAGCCTGAGCAGCACAcccAGC
GCCCTGGGAAAGCTGCAGGACGTGGTCAACcacAATGCCCAGGCACTGAACACCC
TGGTCAAGCAGCTGTCCTCCaagTTCGGCGCCATCAGCTCTGTGCTGAACGATATC
CTGAGCAGACTGGACccccctGAAGCCGAGGTGCAGATCGACAGACTGATCACCGG
AAGGCTGCAGTCCCTGCAGACCTACGTTACCCAGCAGCTGATCAGAGCCGCCGA
GATTAGAGCCTCTGCCAATCTGGCCGCCACCAAGATGTCTGAGTGTGTGCTGGGC
CAGAGCAAGAGAGTGGACTTTTGCGGCAAGGGCTACCACCTGATGAGCTTCCCT
CAGTCTGCCCCTCACGGCGTGGTGTTTCTGCACGTGACATACGTGCCCGCTCAAG
AGAAGAATTTCACCACCGCTCCAGCCATCTGCCACGACGGCAAAGCCCACTTTCC
TAGAGAAGGCGTGTTCGTGTCCAACGGCACCCATTGGTTCGTGACCCAGCGGAA
CTTCTACGAGCCCCAGATCATCACCACCGACAACACCTTCGTGTCTGGCAACTGC
GACGTCGTGATCGGCATTGTGAACAATACCGTGTACGACCCTCTGCAGCCCGAGC
TGGACAGCTTCAAAGAGGAACTGGATAAGTACTTTAAGAACCACACAAGCCCCG
ACGTGGACCTGGGCGATATCAGCGGAATCAATGCCAGCGTCGTGAACATCCAGA
AAGAGATCGACCGGCTGAACGAGGTGGCCAAGAATCTGAACGAGAGCCTGATCG
ACCTGCAAGAACTGGGGAAGTACGAGCAGTACATCAAGTGGCCC
Amino Acid Sequence of BA.2 (Signal peptide is in bold and underlined; Spike
ectodomain is underlined): SEQ ID NO: 39
MFVFLVLLPLVSSQCVNLITRTQSYTNSFTRGVYYPDKVFRSSVLHSTQDLFLPFFSN
VTWFHAIHVSGTNGTKRFDNPVLPFNDGVYFASTEKSNIIRGWIFGTTLDSKTQSLLI
VNNATNVVIKVCEFQFCNDPFLDVYYHKNNKSWMESEFRVYSSANNCTFEYVSQPF
LMDLEGKQGNFKNLREFVFKNIDGYFKIYSKHTPINLGRDLPQGFSALEPLVDLPIGIN
ITRFQTLLALHRSYLTPGDSSSGWTAGAAAYYVGYLQPRTFLLKYNENGTITDAVDC
ALDPLSETKCTLKSFTVEKGIYQTSNFRVQPTESIVRFPNITNLCPFDEVFNATRFASV
YAWNRKRISNCVADYSVLYNFAPFFAFKCYGVSPTKLNDLCFTNVYADSFVIRGNE
VSQIAPGQTGNIADYNYKLPDDFTGCVIAWNSNKLDSKVGGNYNYLYRLFRKSNLK
PFERDISTEIYQAGNKPCNGVAGFNCYFPLRSYGFRPTYGVGHQPYRVVVLSFELLH
APATVCGPKKSTNLVKNKCVNFNFNGLTGTGVLTESNKKFLPFQQFGRDIADTTDA
VRDPQTLEILDITPCSFGGVSVITPGTNTSNQVAVLYQGVNCTEVPVAIHADQLTPTW
RVYSTGSNVFQTRAGCLIGAEYVNNSYECDIPIGAGICASYQTQTKSHASVASQSIIAY
TMSLGAENSVAYSNNSIAIPTNFTISVTTEILPVSMTKTSVDCTMYICGDSTECSNLLL
QYGSFCTQLKRALTGIAVEQDKNTQEVFAQVKQIYKTPPIKYFGGFNFSQILPDPSKP
SKRSPIEDLLFNKVTLADAGFIKQYGDCLGDIAARDLICAQKENGLTVLPPLLTDEMI
AQYTSALLAGTITSGWTFGAGPALQIPFPMQMAYRENGIGVTQNVLYENQKLIANQF
NSAIGKIQDSLSSTPSALGKLQDVVNHNAQALNTLVKQLSSKFGAISSVLNDILSRLD
PPEAEVQIDRLITGRLQSLQTYVTQQLIRAAEIRASANLAATKMSECVLGQSKRVDFC
GKGYHLMSFPQSAPHGVVFLHVTYVPAQEKNFTTAPAICHDGKAHFPREGVFVSNG
THWFVTQRNFYEPQIITTDNTFVSGNCDVVIGIVNNTVYDPLQPELDSFKEELDKYFK
NHTSPDVDLGDISGINASVVNIQKEIDRLNEVAKNLNESLIDLQELGKYEQYIKWP
Nucleotide Sequence of BA.2 (Spike ectodomain without signal peptide): SEQ
ID NO: 40
CAGTGTGTGAACCTGatcACAAGAACCCAGagcTACACCAACAGCTTTACCAGAGG
CGTGTACTACCCCGACAAGGTGTTCAGATCCAGCGTGCTGCACTCTACCCAGGAC
CTGTTCCTGCCTTTCTTCAGCAACGTGACCTGGTTCCACGCCATCCACGTGTCCGG
CACCAATGGCACCAAGAGATTCGACAACCCCGTGCTGCCCTTCAACGACGGGGT
GTACTTTGCCAGCACCGAGAAGTCCAACATCATCAGAGGCTGGATCTTCGGCACC
ACACTGGACAGCAAGACCCAGAGCCTGCTGATCGTGAACAACGCCACCAACGTG
GTCATCAAAGTGTGCGAGTTCCAGTTCTGCAACGACCCCTTCCTGgacGTCTACTA
TCACAAGAACAACAAGAGCTGGATGGAAAGCGAGTTCCGGGTGTACAGCAGCGC
CAACAACTGCACCTTCGAGTACGTGTCCCAGCCTTTCCTGATGGACCTGGAAGGC
AAGCAGGGCAACTTCAAGAACCTGCGCGAGTTCGTGTTCAAGAACATCGACGGC
TACTTCAAGATCTACAGCAAGCACACCCCTATCAACCTCggcCGGGATCTGCCTCA
GGGCTTCTCTGCTCTGGAACCCCTGGTGGATCTGCCCATCGGCATCAACATCACC
CGGTTTCAGACACTGCTGGCCCTGCACAGAAGCTACCTGACACCTGGCGATAGC
AGCAGCGGATGGACAGCTGGTGCCGCCGCTTACTATGTGGGCTACCTGCAGCCT
AGAACCTTTCTGCTGAAGTACAACGAGAACGGCACCATCACCGACGCCGTGGAT
TGTGCTCTGGATCCTCTGAGCGAGACAAAGTGCACCCTGAAGTCCTTCACCGTGG
AgAAGGGCATCTACCAGACCAGCAACTTCCGGGTGCAGCCCACCGAATCCATCGT
GCGGTTCCCCAATATCACCAATCTGTGCCCCTTCgacGAGGTGTTCAATGCCACCA
GATTCGCCTCTGTGTACGCCTGGAACCGGAAGCGGATCAGCAATTGCGTGGCCG
ACTACTCCGTGCTGTACAACttcGCCcctTTCttcgccTTCAAGTGCTACGGCGTGTCCCC
TACCAAGCTGAACGACCTGTGCTTCACAAACGTGTACGCCGACAGCTTCGTGATC
CGGGGAaacGAAGTGagcCAGATTGCCCCTGGACAGACAGGCaacATCGCCGACTAC
AACTACAAGCTGCCCGACGACTTCACCGGCTGTGTGATTGCCTGGAACAGCAACa
agCTGGACTCCAAAGTCGGCGGCAACTACAATTACCTGTACCGGCTGTTCCGGAA
GTCCAATCTGAAGCCCTTCGAGCGGGACATCTCCACCGAGATCTATCAGGCCGGC
aacaagCCTTGTAACGGCGTGgccGGCTTCAACTGCTACTTCCCACTGeggTCCTACGG
CTTTcggCCCACAtacGGCGTGGGCcacCAGCCCTACAGAGTGGTGGTGCTGAGCTTC
GAACTGCTGCATGCCCCTGCCACAGTGTGCGGCCCTAAGAAgAGCACCAATCTCG
TGAAGAACAAATGCGTGAACTTCAACTTCAACGGCCTGACCGGCACCGGCGTGC
TGACAGAGAGCAACAAGAAGTTCCTGCCATTCCAGCAGTTTGGCCGGGATATCG
CCGATACCACAGACGCCGTTAGAGATCCCCAGACACTGGAAATCCTGGACATCA
CCCCTTGCAGCTTCGGCGGAGTGTCTGTGATCACCCCTGGCACCAACACCAGCAA
TCAGGTGGCAGTGCTGTACCAGggcGTGAACTGTACCGAAGTGCCCGTGGCCATT
CACGCCGATCAGCTGACACCTACATGGCGGGTGTACTCCACCGGCAGCAATGTG
TTTCAGACCAGAGCCGGCTGTCTGATCGGAGCCGAGtacGTGAACAATAGCTACG
AGTGCGACATCCCCATCGGCGCTGGCATCTGTGCCAGCTACCAGACACAGACAaa
gAGCcacGCCTCTGTGGCCAGCCAGAGCATCATTGCCTACACAATGTCTCTGGGCG
CCGAGAACAGCGTGGCCTACTCCAACAACTCTATCGCTATCCCCACCAACTTCAC
CATCAGCGTGACCACAGAGATCCTGCCTGTGTCCATGACCAAGACCAGCGTGGA
CTGCACCATGTACATCTGCGGCGATTCCACCGAGTGCTCCAACCTGCTGCTGCAG
TACGGCAGCTTCTGCACCCAGCTGaagAGAGCCCTGACAGGGATCGCCGTGGAAC
AGGACAAGAACACCCAAGAGGTGTTCGCCCAAGTGAAGCAGATCTACAAGACCC
CTCCTATCAAGtacTTCGGCGGCTTCAATTTCAGCCAGATTCTGCCCGATCCTAGCA
AGCCCAGCAAGCGGAGCcctATCGAGGACCTGCTGTTCAACAAAGTGACACTGGC
CGACGCCGGCTTCATCAAGCAGTATGGCGATTGTCTGGGCGACATTGCCGCCAG
GGATCTGATTTGCGCCCAGAAGTTTAACGGACTGACAGTGCTGCCTCCTCTGCTG
ACCGATGAGATGATCGCCCAGTACACATCTGCCCTGCTGGCCGGCACAATCACA
AGCGGCTGGACATTTGGAGCTGGCcctGCTCTGCAGATCCCCTTTccaATGCAGATG
GCCTACCGGTTCAACGGCATCGGAGTGACCCAGAATGTGCTGTACGAGAACCAG
AAGCTGATCGCCAACCAGTTCAACAGCGCCATCGGCAAGATCCAGGACAGCCTG
AGCAGCACAcccAGCGCCCTGGGAAAGCTGCAGGACGTGGTCAACcacAATGCCCA
GGCACTGAACACCCTGGTCAAGCAGCTGTCCTCCaagTTCGGCGCCATCAGCTCTG
TGCTGAACGATATCCTGAGCAGACTGGACccccctGAAGCCGAGGTGCAGATCGAC
AGACTGATCACCGGAAGGCTGCAGTCCCTGCAGACCTACGTTACCCAGCAGCTG
ATCAGAGCCGCCGAGATTAGAGCCTCTGCCAATCTGGCCGCCACCAAGATGTCT
GAGTGTGTGCTGGGCCAGAGCAAGAGAGTGGACTTTTGCGGCAAGGGCTACCAC
CTGATGAGCTTCCCTCAGTCTGCCCCTCACGGCGTGGTGTTTCTGCACGTGACAT
ACGTGCCCGCTCAAGAGAAGAATTTCACCACCGCTCCAGCCATCTGCCACGACG
GCAAAGCCCACTTTCCTAGAGAAGGCGTGTTCGTGTCCAACGGCACCCATTGGTT
CGTGACCCAGCGGAACTTCTACGAGCCCCAGATCATCACCACCGACAACACCTTC
GTGTCTGGCAACTGCGACGTCGTGATCGGCATTGTGAACAATACCGTGTACGACC
CTCTGCAGCCCGAGCTGGACAGCTTCAAAGAGGAACTGGATAAGTACTTTAAGA
ACCACACAAGCCCCGACGTGGACCTGGGCGATATCAGCGGAATCAATGCCAGCG
TCGTGAACATCCAGAAAGAGATCGACCGGCTGAACGAGGTGGCCAAGAATCTGA
ACGAGAGCCTGATCGACCTGCAAGAACTGGGGAAGTACGAGCAGTACATCAAGT
GGCCC
Amino Acid Sequence of BA.2 (Spike ectodomain without signal peptide): SEQ ID
NO: 41
QCVNLITRTQSYTNSFTRGVYYPDKVFRSSVLHSTQDLFLPFFSNVTWFHAIHVSGTN
GTKRFDNPVLPFNDGVYFASTEKSNIIRGWIFGTTLDSKTQSLLIVNNATNVVIKVCEF
QFCNDPFLDVYYHKNNKSWMESEFRVYSSANNCTFEYVSQPFLMDLEGKQGNFKN
LREFVFKNIDGYFKIYSKHTPINLGRDLPQGFSALEPLVDLPIGINITRFQTLLALHRSY
LTPGDSSSGWTAGAAAYYVGYLQPRTFLLKYNENGTITDAVDCALDPLSETKCTLKS
FTVEKGIYQTSNFRVQPTESIVRFPNITNLCPFDEVFNATRFASVYAWNRKRISNCVA
DYSVLYNFAPFFAFKCYGVSPTKLNDLCFTNVYADSFVIRGNEVSQIAPGQTGNIAD
YNYKLPDDFTGCVIAWNSNKLDSKVGGNYNYLYRLFRKSNLKPFERDISTEIYQAGN
KPCNGVAGFNCYFPLRSYGFRPTYGVGHQPYRVVVLSFELLHAPATVCGPKKSTNL
VKNKCVNFNFNGLTGTGVLTESNKKFLPFQQFGRDIADTTDAVRDPQTLEILDITPCS
FGGVSVITPGTNTSNQVAVLYQGVNCTEVPVAIHADQLTPTWRVYSTGSNVFQTRA
GCLIGAEYVNNSYECDIPIGAGICASYQTQTKSHASVASQSIIAYTMSLGAENSVAYS
NNSIAIPTNFTISVTTEILPVSMTKTSVDCTMYICGDSTECSNLLLQYGSFCTQLKRAL
TGIAVEQDKNTQEVFAQVKQIYKTPPIKYFGGFNFSQILPDPSKPSKRSPIEDLLFNKV
TLADAGFIKQYGDCLGDIAARDLICAQKFNGLTVLPPLLTDEMIAQYTSALLAGTITS
GWTFGAGPALQIPFPMQMAYRFNGIGVTQNVLYENQKLIANQFNSAIGKIQDSLSSTP
SALGKLQDVVNHNAQALNTLVKQLSSKFGAISSVLNDILSRLDPPEAEVQIDRLITGR
LQSLQTYVTQQLIRAAEIRASANLAATKMSECVLGQSKRVDFCGKGYHLMSFPQSAP
HGVVFLHVTYVPAQEKNFTTAPAICHDGKAHFPREGVFVSNGTHWFVTQRNFYEPQ
IITTDNTFVSGNCDVVIGIVNNTVYDPLQPELDSFKEELDKYFKNHTSPDVDLGDISGI
NASVVNIQKEIDRLNEVAKNLNESLIDLQELGKYEQYIKWP
Nucleotide Sequence of BA.2 S371 S373 S375 (Signal peptide is in bold and
underlined; GS linker is in italics; TM and CT From F is in bold and double
underlined; Spike ectodomain is underlined; S371 S373 S375 is in bold, italics
and underlined): SEQ ID NO: 42
ATGTTCGTGTTTCTGGTGCTGCTGCCTCTGGTGTCCAGCCAGTGTGTGAACCT
GatcACAAGAACCCAGagcTACACCAACAGCTTTACCAGAGGCGTGTACTACCCCG
ACAAGGTGTTCAGATCCAGCGTGCTGCACTCTACCCAGGACCTGTTCCTGCCTTT
CTTCAGCAACGTGACCTGGTTCCACGCCATCCACGTGTCCGGCACCAATGGCACC
AAGAGATTCGACAACCCCGTGCTGCCCTTCAACGACGGGGTGTACTTTGCCAGCA
CCGAGAAGTCCAACATCATCAGAGGCTGGATCTTCGGCACCACACTGGACAGCA
AGACCCAGAGCCTGCTGATCGTGAACAACGCCACCAACGTGGTCATCAAAGTGT
GCGAGTTCCAGTTCTGCAACGACCCCTTCCTGgacGTCTACTATCACAAGAACAAC
AAGAGCTGGATGGAAAGCGAGTTCCGGGTGTACAGCAGCGCCAACAACTGCACC
TTCGAGTACGTGTCCCAGCCTTTCCTGATGGACCTGGAAGGCAAGCAGGGCAACT
TCAAGAACCTGCGCGAGTTCGTGTTCAAGAACATCGACGGCTACTTCAAGATCTA
CAGCAAGCACACCCCTATCAACCTCggcCGGGATCTGCCTCAGGGCTTCTCTGCTC
TGGAACCCCTGGTGGATCTGCCCATCGGCATCAACATCACCCGGTTTCAGACACT
GCTGGCCCTGCACAGAAGCTACCTGACACCTGGCGATAGCAGCAGCGGATGGAC
AGCTGGTGCCGCCGCTTACTATGTGGGCTACCTGCAGCCTAGAACCTTTCTGCTG
AAGTACAACGAGAACGGCACCATCACCGACGCCGTGGATTGTGCTCTGGATCCT
CTGAGCGAGACAAAGTGCACCCTGAAGTCCTTCACCGTGGAgAAGGGCATCTAC
CAGACCAGCAACTTCCGGGTGCAGCCCACCGAATCCATCGTGCGGTTCCCCAATA
TCACCAATCTGTGCCCCTTCgacGAGGTGTTCAATGCCACCAGATTCGCCTCTGTG
TACGCCTGGAACCGGAAGCGGATCAGCAATTGCGTGGCCGACTACTCCGTGCTG
TACAACtccGCCagcTTCagcgccTTCAAGTGCTACGGCGTGTCCCCTACCAAGCTGAA
CGACCTGTGCTTCACAAACGTGTACGCCGACAGCTTCGTGATCCGGGGAaacGAA
GTGagcCAGATTGCCCCTGGACAGACAGGCaacATCGCCGACTACAACTACAAGCT
GCCCGACGACTTCACCGGCTGTGTGATTGCCTGGAACAGCAACaagCTGGACTCCA
AAGTCGGCGGCAACTACAATTACCTGTACCGGCTGTTCCGGAAGTCCAATCTGAA
GCCCTTCGAGCGGGACATCTCCACCGAGATCTATCAGGCCGGCaacaagCCTTGTAA
CGGCGTGgccGGCTTCAACTGCTACTTCCCACTGcggTCCTACGGCTTTcggCCCACAt
acGGCGTGGGCcacCAGCCCTACAGAGTGGTGGTGCTGAGCTTCGAACTGCTGCAT
GCCCCTGCCACAGTGTGCGGCCCTAAGAAgAGCACCAATCTCGTGAAGAACAAA
TGCGTGAACTTCAACTTCAACGGCCTGACCGGCACCGGCGTGCTGACAGAGAGC
AACAAGAAGTTCCTGCCATTCCAGCAGTTTGGCCGGGATATCGCCGATACCACA
GACGCCGTTAGAGATCCCCAGACACTGGAAATCCTGGACATCACCCCTTGCAGCT
TCGGCGGAGTGTCTGTGATCACCCCTGGCACCAACACCAGCAATCAGGTGGCAG
TGCTGTACCAGggcGTGAACTGTACCGAAGTGCCCGTGGCCATTCACGCCGATCA
GCTGACACCTACATGGCGGGTGTACTCCACCGGCAGCAATGTGTTTCAGACCAG
AGCCGGCTGTCTGATCGGAGCCGAGtacGTGAACAATAGCTACGAGTGCGACATC
CCCATCGGCGCTGGCATCTGTGCCAGCTACCAGACACAGACAaagAGCcacGCCTCT
GTGGCCAGCCAGAGCATCATTGCCTACACAATGTCTCTGGGCGCCGAGAACAGC
GTGGCCTACTCCAACAACTCTATCGCTATCCCCACCAACTTCACCATCAGCGTGA
CCACAGAGATCCTGCCTGTGTCCATGACCAAGACCAGCGTGGACTGCACCATGT
ACATCTGCGGCGATTCCACCGAGTGCTCCAACCTGCTGCTGCAGTACGGCAGCTT
CTGCACCCAGCTGaagAGAGCCCTGACAGGGATCGCCGTGGAACAGGACAAGAAC
ACCCAAGAGGTGTTCGCCCAAGTGAAGCAGATCTACAAGACCCCTCCTATCAAGt
acTTCGGCGGCTTCAATTTCAGCCAGATTCTGCCCGATCCTAGCAAGCCCAGCAAG
CGGAGCcctATCGAGGACCTGCTGTTCAACAAAGTGACACTGGCCGACGCCGGCTT
CATCAAGCAGTATGGCGATTGTCTGGGCGACATTGCCGCCAGGGATCTGATTTGC
GCCCAGAAGTTTAACGGACTGACAGTGCTGCCTCCTCTGCTGACCGATGAGATGA
TCGCCCAGTACACATCTGCCCTGCTGGCCGGCACAATCACAAGCGGCTGGACATT
TGGAGCTGGCcctGCTCTGCAGATCCCCTTTccaATGCAGATGGCCTACCGGTTCAA
CGGCATCGGAGTGACCCAGAATGTGCTGTACGAGAACCAGAAGCTGATCGCCAA
CCAGTTCAACAGCGCCATCGGCAAGATCCAGGACAGCCTGAGCAGCACAcccAGC
GCCCTGGGAAAGCTGCAGGACGTGGTCAACcacAATGCCCAGGCACTGAACACCC
TGGTCAAGCAGCTGTCCTCCaagTTCGGCGCCATCAGCTCTGTGCTGAACGATATC
CTGAGCAGACTGGACccccctGAAGCCGAGGTGCAGATCGACAGACTGATCACCGG
AAGGCTGCAGTCCCTGCAGACCTACGTTACCCAGCAGCTGATCAGAGCCGCCGA
GATTAGAGCCTCTGCCAATCTGGCCGCCACCAAGATGTCTGAGTGTGTGCTGGGC
CAGAGCAAGAGAGTGGACTTTTGCGGCAAGGGCTACCACCTGATGAGCTTCCCT
CAGTCTGCCCCTCACGGCGTGGTGTTTCTGCACGTGACATACGTGCCCGCTCAAG
AGAAGAATTTCACCACCGCTCCAGCCATCTGCCACGACGGCAAAGCCCACTTTCC
TAGAGAAGGCGTGTTCGTGTCCAACGGCACCCATTGGTTCGTGACCCAGCGGAA
CTTCTACGAGCCCCAGATCATCACCACCGACAACACCTTCGTGTCTGGCAACTGC
GACGTCGTGATCGGCATTGTGAACAATACCGTGTACGACCCTCTGCAGCCCGAGC
TGGACAGCTTCAAAGAGGAACTGGATAAGTACTTTAAGAACCACACAAGCCCCG
ACGTGGACCTGGGCGATATCAGCGGAATCAATGCCAGCGTCGTGAACATCCAGA
AAGAGATCGACCGGCTGAACGAGGTGGCCAAGAATCTGAACGAGAGCCTGATCG
ACCTGCAAGAACTGGGGAAGTACGAGCAGTACATCAAGTGGCCCggcggaggtgggtcg
CTCATAACATACATCGTCCTGACTATAATCAGCTTGGTATTTGGTATTTTGTC
TTTGATTCTTGCATGCTATTTGATGTATAAACAGAAAGCTCAGCAGAAGACT
CTCCTGTGGCTCGGTAACAACACACTCGACCAGATGAGAGCAACTACAAAG
ATGTGA
Amino Acid Sequence of BA.2 S371 S373 S375 (Signal peptide is in bold and
underlined; GS linker is in italics; TM and CT From F is in bold and double
underlined; Spike ectodomain is underlined; S371 S373 S375 is in bold, italics and
underlined): SEQ ID NO: 43
MFVFLVLLPLVSSQCVNLITRTQSYTNSFTRGVYYPDKVFRSSVLHSTQDLFLPFFSN
VTWFHAIHVSGTNGTKRFDNPVLPFNDGVYFASTEKSNIIRGWIFGTTLDSKTQSLLI
VNNATNVVIKVCEFQFCNDPFLDVYYHKNNKSWMESEFRVYSSANNCTFEYVSQPF
LMDLEGKQGNFKNLREFVFKNIDGYFKIYSKHTPINLGRDLPQGFSALEPLVDLPIGIN
ITRFQTLLALHRSYLTPGDSSSGWTAGAAAYYVGYLQPRTFLLKYNENGTITDAVDC
ALDPLSETKCTLKSFTVEKGIYQTSNFRVQPTESIVRFPNITNLCPFDEVFNATRFASV
YAWNRKRISNCVADYSVLYNSASFSAFKCYGVSPTKLNDLCFTNVYADSFVIRGNE
VSQIAPGQTGNIADYNYKLPDDFTGCVIAWNSNKLDSKVGGNYNYLYRLFRKSNLK
PFERDISTEIYQAGNKPCNGVAGFNCYFPLRSYGFRPTYGVGHQPYRVVVLSFELLH
APATVCGPKKSTNLVKNKCVNFNFNGLTGTGVLTESNKKFLPFQQFGRDIADTTDA
VRDPQTLEILDITPCSFGGVSVITPGTNTSNQVAVLYQGVNCTEVPVAIHADQLTPTW
RVYSTGSNVFQTRAGCLIGAEYVNNSYECDIPIGAGICASYQTQTKSHASVASQSIIAY
TMSLGAENSVAYSNNSIAIPTNFTISVTTEILPVSMTKTSVDCTMYICGDSTECSNLLL
QYGSFCTQLKRALTGIAVEQDKNTQEVFAQVKQIYKTPPIKYFGGFNFSQILPDPSKP
SKRSPIEDLLFNKVTLADAGFIKQYGDCLGDIAARDLICAQKFNGLTVLPPLLTDEMI
AQYTSALLAGTITSGWTFGAGPALQIPFPMQMAYRENGIGVTQNVLYENQKLIANQF
NSAIGKIQDSLSSTPSALGKLQDVVNHNAQALNTLVKQLSSKFGAISSVLNDILSRLD
PPEAEVQIDRLITGRLQSLQTYVTQQLIRAAEIRASANLAATKMSECVLGQSKRVDFC
GKGYHLMSFPQSAPHGVVFLHVTYVPAQEKNFTTAPAICHDGKAHFPREGVFVSNG
THWFVTQRNFYEPQIITTDNTFVSGNCDVVIGIVNNTVYDPLQPELDSFKEELDKYFK
NHTSPDVDLGDISGINASVVNIQKEIDRLNEVAKNLNESLIDLQELGKYEQYIKWPGG
GGSLITYIVLTIISLVFGILSLILACYLMYKQKAQQKTLLWLGNNTLDQMRATTK
M*
Nucleotide Sequence of BA.2 S371 S373 S375 (Signal peptide is in bold and
underlined; Spike ectodomain is underlined; S371 S373 S375 is in bold, italics
and underlined): SEQ ID NO: 44
ATGTTCGTGTTTCTGGTGCTGCTGCCTCTGGTGTCCAGCCAGTGTGTGAACCT
GatcACAAGAACCCAGagcTACACCAACAGCTTTACCAGAGGCGTGTACTACCCCG
ACAAGGTGTTCAGATCCAGCGTGCTGCACTCTACCCAGGACCTGTTCCTGCCTTT
CTTCAGCAACGTGACCTGGTTCCACGCCATCCACGTGTCCGGCACCAATGGCACC
AAGAGATTCGACAACCCCGTGCTGCCCTTCAACGACGGGGTGTACTTTGCCAGCA
CCGAGAAGTCCAACATCATCAGAGGCTGGATCTTCGGCACCACACTGGACAGCA
AGACCCAGAGCCTGCTGATCGTGAACAACGCCACCAACGTGGTCATCAAAGTGT
GCGAGTTCCAGTTCTGCAACGACCCCTTCCTGgacGTCTACTATCACAAGAACAAC
AAGAGCTGGATGGAAAGCGAGTTCCGGGTGTACAGCAGCGCCAACAACTGCACC
TTCGAGTACGTGTCCCAGCCTTTCCTGATGGACCTGGAAGGCAAGCAGGGCAACT
TCAAGAACCTGCGCGAGTTCGTGTTCAAGAACATCGACGGCTACTTCAAGATCTA
CAGCAAGCACACCCCTATCAACCTCggcCGGGATCTGCCTCAGGGCTTCTCTGCTC
TGGAACCCCTGGTGGATCTGCCCATCGGCATCAACATCACCCGGTTTCAGACACT
GCTGGCCCTGCACAGAAGCTACCTGACACCTGGCGATAGCAGCAGCGGATGGAC
AGCTGGTGCCGCCGCTTACTATGTGGGCTACCTGCAGCCTAGAACCTTTCTGCTG
AAGTACAACGAGAACGGCACCATCACCGACGCCGTGGATTGTGCTCTGGATCCT
CTGAGCGAGACAAAGTGCACCCTGAAGTCCTTCACCGTGGAgAAGGGCATCTAC
CAGACCAGCAACTTCCGGGTGCAGCCCACCGAATCCATCGTGCGGTTCCCCAATA
TCACCAATCTGTGCCCCTTCgacGAGGTGTTCAATGCCACCAGATTCGCCTCTGTG
TACGCCTGGAACCGGAAGCGGATCAGCAATTGCGTGGCCGACTACTCCGTGCTG
TACAACtccGCCagcTTCagcgccTTCAAGTGCTACGGCGTGTCCCCTACCAAGCTGAA
CGACCTGTGCTTCACAAACGTGTACGCCGACAGCTTCGTGATCCGGGGAaacGAA
GTGagcCAGATTGCCCCTGGACAGACAGGCaacATCGCCGACTACAACTACAAGCT
GCCCGACGACTTCACCGGCTGTGTGATTGCCTGGAACAGCAACaagCTGGACTCCA
AAGTCGGCGGCAACTACAATTACCTGTACCGGCTGTTCCGGAAGTCCAATCTGAA
GCCCTTCGAGCGGGACATCTCCACCGAGATCTATCAGGCCGGCaacaagCCTTGTAA
CGGCGTGgccGGCTTCAACTGCTACTTCCCACTGcggTCCTACGGCTTTcggCCCACAt
acGGCGTGGGCcacCAGCCCTACAGAGTGGTGGTGCTGAGCTTCGAACTGCTGCAT
GCCCCTGCCACAGTGTGCGGCCCTAAGAAgAGCACCAATCTCGTGAAGAACAAA
TGCGTGAACTTCAACTTCAACGGCCTGACCGGCACCGGCGTGCTGACAGAGAGC
AACAAGAAGTTCCTGCCATTCCAGCAGTTTGGCCGGGATATCGCCGATACCACA
GACGCCGTTAGAGATCCCCAGACACTGGAAATCCTGGACATCACCCCTTGCAGCT
TCGGCGGAGTGTCTGTGATCACCCCTGGCACCAACACCAGCAATCAGGTGGCAG
TGCTGTACCAGggcGTGAACTGTACCGAAGTGCCCGTGGCCATTCACGCCGATCA
GCTGACACCTACATGGCGGGTGTACTCCACCGGCAGCAATGTGTTTCAGACCAG
AGCCGGCTGTCTGATCGGAGCCGAGtacGTGAACAATAGCTACGAGTGCGACATC
CCCATCGGCGCTGGCATCTGTGCCAGCTACCAGACACAGACAaagAGCcacGCCTCT
GTGGCCAGCCAGAGCATCATTGCCTACACAATGTCTCTGGGCGCCGAGAACAGC
GTGGCCTACTCCAACAACTCTATCGCTATCCCCACCAACTTCACCATCAGCGTGA
CCACAGAGATCCTGCCTGTGTCCATGACCAAGACCAGCGTGGACTGCACCATGT
ACATCTGCGGCGATTCCACCGAGTGCTCCAACCTGCTGCTGCAGTACGGCAGCTT
CTGCACCCAGCTGaagAGAGCCCTGACAGGGATCGCCGTGGAACAGGACAAGAAC
ACCCAAGAGGTGTTCGCCCAAGTGAAGCAGATCTACAAGACCCCTCCTATCAAGt
acTTCGGCGGCTTCAATTTCAGCCAGATTCTGCCCGATCCTAGCAAGCCCAGCAAG
CGGAGCcctATCGAGGACCTGCTGTTCAACAAAGTGACACTGGCCGACGCCGGCTT
CATCAAGCAGTATGGCGATTGTCTGGGCGACATTGCCGCCAGGGATCTGATTTGC
GCCCAGAAGTTTAACGGACTGACAGTGCTGCCTCCTCTGCTGACCGATGAGATGA
TCGCCCAGTACACATCTGCCCTGCTGGCCGGCACAATCACAAGCGGCTGGACATT
TGGAGCTGGCcctGCTCTGCAGATCCCCTTTccaATGCAGATGGCCTACCGGTTCAA
CGGCATCGGAGTGACCCAGAATGTGCTGTACGAGAACCAGAAGCTGATCGCCAA
CCAGTTCAACAGCGCCATCGGCAAGATCCAGGACAGCCTGAGCAGCACAcccAGC
GCCCTGGGAAAGCTGCAGGACGTGGTCAACcacAATGCCCAGGCACTGAACACCC
TGGTCAAGCAGCTGTCCTCCaagTTCGGCGCCATCAGCTCTGTGCTGAACGATATC
CTGAGCAGACTGGACccccctGAAGCCGAGGTGCAGATCGACAGACTGATCACCGG
AAGGCTGCAGTCCCTGCAGACCTACGTTACCCAGCAGCTGATCAGAGCCGCCGA
GATTAGAGCCTCTGCCAATCTGGCCGCCACCAAGATGTCTGAGTGTGTGCTGGGC
CAGAGCAAGAGAGTGGACTTTTGCGGCAAGGGCTACCACCTGATGAGCTTCCCT
CAGTCTGCCCCTCACGGCGTGGTGTTTCTGCACGTGACATACGTGCCCGCTCAAG
AGAAGAATTTCACCACCGCTCCAGCCATCTGCCACGACGGCAAAGCCCACTTTCC
TAGAGAAGGCGTGTTCGTGTCCAACGGCACCCATTGGTTCGTGACCCAGCGGAA
CTTCTACGAGCCCCAGATCATCACCACCGACAACACCTTCGTGTCTGGCAACTGC
GACGTCGTGATCGGCATTGTGAACAATACCGTGTACGACCCTCTGCAGCCCGAGC
TGGACAGCTTCAAAGAGGAACTGGATAAGTACTTTAAGAACCACACAAGCCCCG
ACGTGGACCTGGGCGATATCAGCGGAATCAATGCCAGCGTCGTGAACATCCAGA
AAGAGATCGACCGGCTGAACGAGGTGGCCAAGAATCTGAACGAGAGCCTGATCG
ACCTGCAAGAACTGGGGAAGTACGAGCAGTACATCAAGTGGCCC
Amino Acid Sequence of BA.2 S371 S373 S375 (Signal peptide is in bold and
underlined; Spike ectodomain is underlined; S371 S373 S375 is in bold, italics
and underlined): SEQ ID NO: 45
MFVFLVLLPLVSSQCVNLITRTQSYTNSFTRGVYYPDKVFRSSVLHSTQDLFLPFFSN
VTWFHAIHVSGTNGTKRFDNPVLPFNDGVYFASTEKSNIIRGWIFGTTLDSKTQSLLI
VNNATNVVIKVCEFQFCNDPFLDVYYHKNNKSWMESEFRVYSSANNCTFEYVSQPF
LMDLEGKQGNFKNLREFVFKNIDGYFKIYSKHTPINLGRDLPQGFSALEPLVDLPIGIN
ITRFQTLLALHRSYLTPGDSSSGWTAGAAAYYVGYLQPRTFLLKYNENGTITDAVDC
ALDPLSETKCTLKSFTVEKGIYQTSNFRVQPTESIVRFPNITNLCPFDEVFNATRFASV
YAWNRKRISNCVADYSVLYNSASFSAFKCYGVSPTKLNDLCFTNVYADSFVIRGNE
VSQIAPGQTGNIADYNYKLPDDFTGCVIAWNSNKLDSKVGGNYNYLYRLFRKSNLK
PFERDISTEIYQAGNKPCNGVAGFNCYFPLRSYGFRPTYGVGHQPYRVVVLSFELLH
APATVCGPKKSTNLVKNKCVNFNFNGLTGTGVLTESNKKFLPFQQFGRDIADTTDA
VRDPQTLEILDITPCSFGGVSVITPGTNTSNQVAVLYQGVNCTEVPVAIHADQLTPTW
RVYSTGSNVFQTRAGCLIGAEYVNNSYECDIPIGAGICASYQTQTKSHASVASQSIIAY
TMSLGAENSVAYSNNSIAIPTNFTISVTTEILPVSMTKTSVDCTMYICGDSTECSNLLL
QYGSFCTQLKRALTGIAVEQDKNTQEVFAQVKQIYKTPPIKYFGGFNFSQILPDPSKP
SKRSPIEDLLFNKVTLADAGFIKQYGDCLGDIAARDLICAQKFNGLTVLPPLLTDEMI
AQYTSALLAGTITSGWTFGAGPALQIPFPMQMAYRFNGIGVTQNVLYENQKLIANQF
NSAIGKIQDSLSSTPSALGKLQDVVNHNAQALNTLVKQLSSKFGAISSVLNDILSRLD
PPEAEVQIDRLITGRLQSLQTYVTQQLIRAAEIRASANLAATKMSECVLGQSKRVDFC
GKGYHLMSFPQSAPHGVVFLHVTYVPAQEKNFTTAPAICHDGKAHFPREGVFVSNG
THWFVTQRNFYEPQIITTDNTFVSGNCDVVIGIVNNTVYDPLQPELDSFKEELDKYFK
NHTSPDVDLGDISGINASVVNIQKEIDRLNEVAKNLNESLIDLQELGKYEQYIKWP
Nucleotide Sequence of BA.2 S371 S373 S375 (Spike ectodomain without signal
peptide is underlined; S371 S373 S375 is in bold, italics and underlined): SEQ ID
NO: 46
CAGTGTGTGAACCTGatcACAAGAACCCAGagcTACACCAACAGCTTTACCAGAGG
CGTGTACTACCCCGACAAGGTGTTCAGATCCAGCGTGCTGCACTCTACCCAGGAC
CTGTTCCTGCCTTTCTTCAGCAACGTGACCTGGTTCCACGCCATCCACGTGTCCGG
CACCAATGGCACCAAGAGATTCGACAACCCCGTGCTGCCCTTCAACGACGGGGT
GTACTTTGCCAGCACCGAGAAGTCCAACATCATCAGAGGCTGGATCTTCGGCACC
ACACTGGACAGCAAGACCCAGAGCCTGCTGATCGTGAACAACGCCACCAACGTG
GTCATCAAAGTGTGCGAGTTCCAGTTCTGCAACGACCCCTTCCTGgacGTCTACTA
TCACAAGAACAACAAGAGCTGGATGGAAAGCGAGTTCCGGGTGTACAGCAGCGC
CAACAACTGCACCTTCGAGTACGTGTCCCAGCCTTTCCTGATGGACCTGGAAGGC
AAGCAGGGCAACTTCAAGAACCTGCGCGAGTTCGTGTTCAAGAACATCGACGGC
TACTTCAAGATCTACAGCAAGCACACCCCTATCAACCTCggcCGGGATCTGCCTCA
GGGCTTCTCTGCTCTGGAACCCCTGGTGGATCTGCCCATCGGCATCAACATCACC
CGGTTTCAGACACTGCTGGCCCTGCACAGAAGCTACCTGACACCTGGCGATAGC
AGCAGCGGATGGACAGCTGGTGCCGCCGCTTACTATGTGGGCTACCTGCAGCCT
AGAACCTTTCTGCTGAAGTACAACGAGAACGGCACCATCACCGACGCCGTGGAT
TGTGCTCTGGATCCTCTGAGCGAGACAAAGTGCACCCTGAAGTCCTTCACCGTGG
AgAAGGGCATCTACCAGACCAGCAACTTCCGGGTGCAGCCCACCGAATCCATCGT
GCGGTTCCCCAATATCACCAATCTGTGCCCCTTCgacGAGGTGTTCAATGCCACCA
GATTCGCCTCTGTGTACGCCTGGAACCGGAAGCGGATCAGCAATTGCGTGGCCG
ACTACTCCGTGCTGTACAACtccGCCagcTTCagcgccTTCAAGTGCTACGGCGTGTCC
CCTACCAAGCTGAACGACCTGTGCTTCACAAACGTGTACGCCGACAGCTTCGTGA
TCCGGGGAaacGAAGTGagcCAGATTGCCCCTGGACAGACAGGCaacATCGCCGACT
ACAACTACAAGCTGCCCGACGACTTCACCGGCTGTGTGATTGCCTGGAACAGCA
ACaagCTGGACTCCAAAGTCGGCGGCAACTACAATTACCTGTACCGGCTGTTCCGG
AAGTCCAATCTGAAGCCCTTCGAGCGGGACATCTCCACCGAGATCTATCAGGCC
GGCaacaagCCTTGTAACGGCGTGgccGGCTTCAACTGCTACTTCCCACTGcggTCCTA
CGGCTTTcggCCCACAtacGGCGTGGGCcacCAGCCCTACAGAGTGGTGGTGCTGAGC
TTCGAACTGCTGCATGCCCCTGCCACAGTGTGCGGCCCTAAGAAgAGCACCAATC
TCGTGAAGAACAAATGCGTGAACTTCAACTTCAACGGCCTGACCGGCACCGGCG
TGCTGACAGAGAGCAACAAGAAGTTCCTGCCATTCCAGCAGTTTGGCCGGGATA
TCGCCGATACCACAGACGCCGTTAGAGATCCCCAGACACTGGAAATCCTGGACA
TCACCCCTTGCAGCTTCGGCGGAGTGTCTGTGATCACCCCTGGCACCAACACCAG
CAATCAGGTGGCAGTGCTGTACCAGggcGTGAACTGTACCGAAGTGCCCGTGGCC
ATTCACGCCGATCAGCTGACACCTACATGGCGGGTGTACTCCACCGGCAGCAAT
GTGTTTCAGACCAGAGCCGGCTGTCTGATCGGAGCCGAGtacGTGAACAATAGCTA
CGAGTGCGACATCCCCATCGGCGCTGGCATCTGTGCCAGCTACCAGACACAGAC
AaagAGCcacGCCTCTGTGGCCAGCCAGAGCATCATTGCCTACACAATGTCTCTGGG
CGCCGAGAACAGCGTGGCCTACTCCAACAACTCTATCGCTATCCCCACCAACTTC
ACCATCAGCGTGACCACAGAGATCCTGCCTGTGTCCATGACCAAGACCAGCGTG
GACTGCACCATGTACATCTGCGGCGATTCCACCGAGTGCTCCAACCTGCTGCTGC
AGTACGGCAGCTTCTGCACCCAGCTGaagAGAGCCCTGACAGGGATCGCCGTGGA
ACAGGACAAGAACACCCAAGAGGTGTTCGCCCAAGTGAAGCAGATCTACAAGAC
CCCTCCTATCAAGtacTTCGGCGGCTTCAATTTCAGCCAGATTCTGCCCGATCCTAG
CAAGCCCAGCAAGCGGAGCcctATCGAGGACCTGCTGTTCAACAAAGTGACACTG
GCCGACGCCGGCTTCATCAAGCAGTATGGCGATTGTCTGGGCGACATTGCCGCCA
GGGATCTGATTTGCGCCCAGAAGTTTAACGGACTGACAGTGCTGCCTCCTCTGCT
GACCGATGAGATGATCGCCCAGTACACATCTGCCCTGCTGGCCGGCACAATCAC
AAGCGGCTGGACATTTGGAGCTGGCcctGCTCTGCAGATCCCCTTTccaATGCAGAT
GGCCTACCGGTTCAACGGCATCGGAGTGACCCAGAATGTGCTGTACGAGAACCA
GAAGCTGATCGCCAACCAGTTCAACAGCGCCATCGGCAAGATCCAGGACAGCCT
GAGCAGCACAcccAGCGCCCTGGGAAAGCTGCAGGACGTGGTCAACcacAATGCCC
AGGCACTGAACACCCTGGTCAAGCAGCTGTCCTCCaagTTCGGCGCCATCAGCTCT
GTGCTGAACGATATCCTGAGCAGACTGGACccccctGAAGCCGAGGTGCAGATCGA
CAGACTGATCACCGGAAGGCTGCAGTCCCTGCAGACCTACGTTACCCAGCAGCT
GATCAGAGCCGCCGAGATTAGAGCCTCTGCCAATCTGGCCGCCACCAAGATGTC
TGAGTGTGTGCTGGGCCAGAGCAAGAGAGTGGACTTTTGCGGCAAGGGCTACCA
CCTGATGAGCTTCCCTCAGTCTGCCCCTCACGGCGTGGTGTTTCTGCACGTGACA
TACGTGCCCGCTCAAGAGAAGAATTTCACCACCGCTCCAGCCATCTGCCACGACG
GCAAAGCCCACTTTCCTAGAGAAGGCGTGTTCGTGTCCAACGGCACCCATTGGTT
CGTGACCCAGCGGAACTTCTACGAGCCCCAGATCATCACCACCGACAACACCTTC
GTGTCTGGCAACTGCGACGTCGTGATCGGCATTGTGAACAATACCGTGTACGACC
CTCTGCAGCCCGAGCTGGACAGCTTCAAAGAGGAACTGGATAAGTACTTTAAGA
ACCACACAAGCCCCGACGTGGACCTGGGCGATATCAGCGGAATCAATGCCAGCG
TCGTGAACATCCAGAAAGAGATCGACCGGCTGAACGAGGTGGCCAAGAATCTGA
ACGAGAGCCTGATCGACCTGCAAGAACTGGGGAAGTACGAGCAGTACATCAAGT
GGCCC
Amino Acid Sequence of BA.2 S371 S373 S375 (Spike ectodomain without signal
peptide is underlined; S371 S373 S375 is in bold, italics and underlined): SEQ
ID NO: 47
QCVNLITRTQSYTNSFTRGVYYPDKVFRSSVLHSTQDLFLPFFSNVTWFHAIHVSGTN
GTKRFDNPVLPFNDGVYFASTEKSNIIRGWIFGTTLDSKTQSLLIVNNATNVVIKVCEF
QFCNDPFLDVYYHKNNKSWMESEFRVYSSANNCTFEYVSQPFLMDLEGKQGNFKN
LREFVFKNIDGYFKIYSKHTPINLGRDLPQGFSALEPLVDLPIGINITRFQTLLALHRSY
LTPGDSSSGWTAGAAAYYVGYLQPRTFLLKYNENGTITDAVDCALDPLSETKCTLKS
FTVEKGIYQTSNFRVQPTESIVRFPNITNLCPFDEVFNATRFASVYAWNRKRISNCVA
DYSVLYNSASFSAFKCYGVSPTKLNDLCFTNVYADSFVIRGNEVSQIAPGQTGNIAD
YNYKLPDDFTGCVIAWNSNKLDSKVGGNYNYLYRLFRKSNLKPFERDISTEIYQAGN
KPCNGVAGFNCYFPLRSYGFRPTYGVGHQPYRVVVLSFELLHAPATVCGPKKSTNL
VKNKCVNFNFNGLTGTGVLTESNKKFLPFQQFGRDIADTTDAVRDPQTLEILDITPCS
FGGVSVITPGTNTSNQVAVLYQGVNCTEVPVAIHADQLTPTWRVYSTGSNVFQTRA
GCLIGAEYVNNSYECDIPIGAGICASYQTQTKSHASVASQSIIAYTMSLGAENSVAYS
NNSIAIPTNFTISVTTEILPVSMTKTSVDCTMYICGDSTECSNLLLQYGSFCTQLKRAL
TGIAVEQDKNTQEVFAQVKQIYKTPPIKYFGGFNFSQILPDPSKPSKRSPIEDLLFNKV
TLADAGFIKQYGDCLGDIAARDLICAQKFNGLTVLPPLLTDEMIAQYTSALLAGTITS
GWTFGAGPALQIPFPMQMAYRENGIGVTQNVLYENQKLIANQFNSAIGKIQDSLSSTP
SALGKLQDVVNHNAQALNTLVKQLSSKFGAISSVLNDILSRLDPPEAEVQIDRLITGR
LQSLQTYVTQQLIRAAEIRASANLAATKMSECVLGQSKRVDFCGKGYHLMSFPQSAP
HGVVFLHVTYVPAQEKNFTTAPAICHDGKAHFPREGVFVSNGTHWFVTQRNFYEPQ
IITTDNTFVSGNCDVVIGIVNNTVYDPLQPELDSFKEELDKYFKNHTSPDVDLGDISGI
NASVVNIQKEIDRLNEVAKNLNESLIDLQELGKYEQYIKWP
Nucleotide Sequence of BA.2.12.1 (Signal peptide is in bold and underlined; Spike
protein ectodomain underlined; GS linker is in italics; TM and CT From F in bold
and double underlined): SEQ ID NO: 48
ATGTTCGTGTTTCTGGTGCTGCTGCCTCTGGTGTCCAGCCAGTGTGTGAACCT
GatcACAAGAACCCAGagcTACACCAACAGCTTTACCAGAGGCGTGTACTACCCCG
ACAAGGTGTTCAGATCCAGCGTGCTGCACTCTACCCAGGACCTGTTCCTGCCTTT
CTTCAGCAACGTGACCTGGTTCCACGCCATCCACGTGTCCGGCACCAATGGCACC
AAGAGATTCGACAACCCCGTGCTGCCCTTCAACGACGGGGTGTACTTTGCCAGCA
CCGAGAAGTCCAACATCATCAGAGGCTGGATCTTCGGCACCACACTGGACAGCA
AGACCCAGAGCCTGCTGATCGTGAACAACGCCACCAACGTGGTCATCAAAGTGT
GCGAGTTCCAGTTCTGCAACGACCCCTTCCTGgacGTCTACTATCACAAGAACAAC
AAGAGCTGGATGGAAAGCGAGTTCCGGGTGTACAGCAGCGCCAACAACTGCACC
TTCGAGTACGTGTCCCAGCCTTTCCTGATGGACCTGGAAGGCAAGCAGGGCAACT
TCAAGAACCTGCGCGAGTTCGTGTTCAAGAACATCGACGGCTACTTCAAGATCTA
CAGCAAGCACACCCCTATCAACCTCggcCGGGATCTGCCTCAGGGCTTCTCTGCTC
TGGAACCCCTGGTGGATCTGCCCATCGGCATCAACATCACCCGGTTTCAGACACT
GCTGGCCCTGCACAGAAGCTACCTGACACCTGGCGATAGCAGCAGCGGATGGAC
AGCTGGTGCCGCCGCTTACTATGTGGGCTACCTGCAGCCTAGAACCTTTCTGCTG
AAGTACAACGAGAACGGCACCATCACCGACGCCGTGGATTGTGCTCTGGATCCT
CTGAGCGAGACAAAGTGCACCCTGAAGTCCTTCACCGTGGAgAAGGGCATCTAC
CAGACCAGCAACTTCCGGGTGCAGCCCACCGAATCCATCGTGCGGTTCCCCAATA
TCACCAATCTGTGCCCCTTCgacGAGGTGTTCAATGCCACCAGATTCGCCTCTGTG
TACGCCTGGAACCGGAAGCGGATCAGCAATTGCGTGGCCGACTACTCCGTGCTG
TACAACttcGCCcctTTCttcgccTTCAAGTGCTACGGCGTGTCCCCTACCAAGCTGAAC
GACCTGTGCTTCACAAACGTGTACGCCGACAGCTTCGTGATCCGGGGAaacGAAG
TGagcCAGATTGCCCCTGGACAGACAGGCaacATCGCCGACTACAACTACAAGCTG
CCCGACGACTTCACCGGCTGTGTGATTGCCTGGAACAGCAACaagCTGGACTCCAA
AGTCGGCGGCAACTACAATTACcagTACCGGCTGTTCCGGAAGTCCAATCTGAAG
CCCTTCGAGCGGGACATCTCCACCGAGATCTATCAGGCCGGCaacaagCCTTGTAAC
GGCGTGgccGGCTTCAACTGCTACTTCCCACTGcggTCCTACGGCTTTcggCCCACAta
cGGCGTGGGCcacCAGCCCTACAGAGTGGTGGTGCTGAGCTTCGAACTGCTGCATG
CCCCTGCCACAGTGTGCGGCCCTAAGAAgAGCACCAATCTCGTGAAGAACAAAT
GCGTGAACTTCAACTTCAACGGCCTGACCGGCACCGGCGTGCTGACAGAGAGCA
ACAAGAAGTTCCTGCCATTCCAGCAGTTTGGCCGGGATATCGCCGATACCACAG
ACGCCGTTAGAGATCCCCAGACACTGGAAATCCTGGACATCACCCCTTGCAGCTT
CGGCGGAGTGTCTGTGATCACCCCTGGCACCAACACCAGCAATCAGGTGGCAGT
GCTGTACCAGggcGTGAACTGTACCGAAGTGCCCGTGGCCATTCACGCCGATCAG
CTGACACCTACATGGCGGGTGTACTCCACCGGCAGCAATGTGTTTCAGACCAGA
GCCGGCTGTCTGATCGGAGCCGAGtacGTGAACAATAGCTACGAGTGCGACATCC
CCATCGGCGCTGGCATCTGTGCCAGCTACCAGACACAGACAaagAGCcacGCCTCT
GTGGCCAGCCAGAGCATCATTGCCTACACAATGTCTCTGGGCGCCGAGAACctgGT
GGCCTACTCCAACAACTCTATCGCTATCCCCACCAACTTCACCATCAGCGTGACC
ACAGAGATCCTGCCTGTGTCCATGACCAAGACCAGCGTGGACTGCACCATGTAC
ATCTGCGGCGATTCCACCGAGTGCTCCAACCTGCTGCTGCAGTACGGCAGCTTCT
GCACCCAGCTGaagAGAGCCCTGACAGGGATCGCCGTGGAACAGGACAAGAACA
CCCAAGAGGTGTTCGCCCAAGTGAAGCAGATCTACAAGACCCCTCCTATCAAGtac
TTCGGCGGCTTCAATTTCAGCCAGATTCTGCCCGATCCTAGCAAGCCCAGCAAGC
GGAGCcctATCGAGGACCTGCTGTTCAACAAAGTGACACTGGCCGACGCCGGCTTC
ATCAAGCAGTATGGCGATTGTCTGGGCGACATTGCCGCCAGGGATCTGATTTGCG
CCCAGAAGTTTAACGGACTGACAGTGCTGCCTCCTCTGCTGACCGATGAGATGAT
CGCCCAGTACACATCTGCCCTGCTGGCCGGCACAATCACAAGCGGCTGGACATTT
GGAGCTGGCcctGCTCTGCAGATCCCCTTTccaATGCAGATGGCCTACCGGTTCAAC
GGCATCGGAGTGACCCAGAATGTGCTGTACGAGAACCAGAAGCTGATCGCCAAC
CAGTTCAACAGCGCCATCGGCAAGATCCAGGACAGCCTGAGCAGCACAcccAGCG
CCCTGGGAAAGCTGCAGGACGTGGTCAACcacAATGCCCAGGCACTGAACACCCT
GGTCAAGCAGCTGTCCTCCaagTTCGGCGCCATCAGCTCTGTGCTGAACGATATCC
TGAGCAGACTGGACccccctGAAGCCGAGGTGCAGATCGACAGACTGATCACCGGA
AGGCTGCAGTCCCTGCAGACCTACGTTACCCAGCAGCTGATCAGAGCCGCCGAG
ATTAGAGCCTCTGCCAATCTGGCCGCCACCAAGATGTCTGAGTGTGTGCTGGGCC
AGAGCAAGAGAGTGGACTTTTGCGGCAAGGGCTACCACCTGATGAGCTTCCCTC
AGTCTGCCCCTCACGGCGTGGTGTTTCTGCACGTGACATACGTGCCCGCTCAAGA
GAAGAATTTCACCACCGCTCCAGCCATCTGCCACGACGGCAAAGCCCACTTTCCT
AGAGAAGGCGTGTTCGTGTCCAACGGCACCCATTGGTTCGTGACCCAGCGGAAC
TTCTACGAGCCCCAGATCATCACCACCGACAACACCTTCGTGTCTGGCAACTGCG
ACGTCGTGATCGGCATTGTGAACAATACCGTGTACGACCCTCTGCAGCCCGAGCT
GGACAGCTTCAAAGAGGAACTGGATAAGTACTTTAAGAACCACACAAGCCCCGA
CGTGGACCTGGGCGATATCAGCGGAATCAATGCCAGCGTCGTGAACATCCAGAA
AGAGATCGACCGGCTGAACGAGGTGGCCAAGAATCTGAACGAGAGCCTGATCGA
CCTGCAAGAACTGGGGAAGTACGAGCAGTACATCAAGTGGCCCggcggaggtgggtcgC
TCATAACATACATCGTCCTGACTATAATCAGCTTGGTATTTGGTATTTTGTCT
TTGATTCTTGCATGCTATTTGATGTATAAACAGAAAGCTCAGCAGAAGACTC
TCCTGTGGCTCGGTAACAACACACTCGACCAGATGAGAGCAACTACAAAGA
TGTGA
Amino Acid Sequence of BA.2.12.1 (Signal peptide is in bold and underlined; Spike
protein ectodomain underlined; GS linker is in italics; TM and CT From F in bold
and double underlined): SEQ ID NO: 49
MFVFLVLLPLVSSQCVNLITRTQSYTNSFTRGVYYPDKVFRSSVLHSTQDLFLPFFSN
VTWFHAIHVSGTNGTKRFDNPVLPFNDGVYFASTEKSNIIRGWIFGTTLDSKTQSLLI
VNNATNVVIKVCEFQFCNDPFLDVYYHKNNKSWMESEFRVYSSANNCTFEYVSQPF
LMDLEGKQGNFKNLREFVFKNIDGYFKIYSKHTPINLGRDLPQGFSALEPLVDLPIGIN
ITRFQTLLALHRSYLTPGDSSSGWTAGAAAYYVGYLQPRTFLLKYNENGTITDAVDC
ALDPLSETKCTLKSFTVEKGIYQTSNFRVQPTESIVRFPNITNLCPFDEVFNATRFASV
YAWNRKRISNCVADYSVLYNFAPFFAFKCYGVSPTKLNDLCFTNVYADSFVIRGNE
VSQIAPGQTGNIADYNYKLPDDFTGCVIAWNSNKLDSKVGGNYNYQYRLFRKSNLK
PFERDISTEIYQAGNKPCNGVAGFNCYFPLRSYGFRPTYGVGHQPYRVVVLSFELLH
APATVCGPKKSTNLVKNKCVNFNFNGLTGTGVLTESNKKFLPFQQFGRDIADTTDA
VRDPQTLEILDITPCSFGGVSVITPGTNTSNQVAVLYQGVNCTEVPVAIHADQLTPTW
RVYSTGSNVFQTRAGCLIGAEYVNNSYECDIPIGAGICASYQTQTKSHASVASQSIIAY
TMSLGAENLVAYSNNSIAIPTNFTISVTTEILPVSMTKTSVDCTMYICGDSTECSNLLL
QYGSFCTQLKRALTGIAVEQDKNTQEVFAQVKQIYKTPPIKYFGGFNFSQILPDPSKP
SKRSPIEDLLFNKVTLADAGFIKQYGDCLGDIAARDLICAQKFNGLTVLPPLLTDEMI
AQYTSALLAGTITSGWTFGAGPALQIPFPMQMAYRENGIGVTQNVLYENQKLIANQE
NSAIGKIQDSLSSTPSALGKLQDVVNHNAQALNTLVKQLSSKFGAISSVLNDILSRLD
PPEAEVQIDRLITGRLQSLQTYVTQQLIRAAEIRASANLAATKMSECVLGQSKRVDFC
GKGYHLMSFPQSAPHGVVFLHVTYVPAQEKNFTTAPAICHDGKAHFPREGVFVSNG
THWFVTQRNFYEPQIITTDNTFVSGNCDVVIGIVNNTVYDPLQPELDSFKEELDKYFK
NHTSPDVDLGDISGINASVVNIQKEIDRLNEVAKNLNESLIDLQELGKYEQYIKWPGG
GGSLITYIVLTIISLVFGILSLILACYLMYKQKAQQKTLLWLGNNTLDQMRATTK
M*
Nucleotide Sequence of BA.2.12.1 (Signal peptide is in bold and underlined; Spike
protein ectodomain underlined): SEQ ID NO: 50
ATGTTCGTGTTTCTGGTGCTGCTGCCTCTGGTGTCCAGCCAGTGTGTGAACCT
GatcACAAGAACCCAGagcTACACCAACAGCTTTACCAGAGGCGTGTACTACCCCG
ACAAGGTGTTCAGATCCAGCGTGCTGCACTCTACCCAGGACCTGTTCCTGCCTTT
CTTCAGCAACGTGACCTGGTTCCACGCCATCCACGTGTCCGGCACCAATGGCACC
AAGAGATTCGACAACCCCGTGCTGCCCTTCAACGACGGGGTGTACTTTGCCAGCA
CCGAGAAGTCCAACATCATCAGAGGCTGGATCTTCGGCACCACACTGGACAGCA
AGACCCAGAGCCTGCTGATCGTGAACAACGCCACCAACGTGGTCATCAAAGTGT
GCGAGTTCCAGTTCTGCAACGACCCCTTCCTGgacGTCTACTATCACAAGAACAAC
AAGAGCTGGATGGAAAGCGAGTTCCGGGTGTACAGCAGCGCCAACAACTGCACC
TTCGAGTACGTGTCCCAGCCTTTCCTGATGGACCTGGAAGGCAAGCAGGGCAACT
TCAAGAACCTGCGCGAGTTCGTGTTCAAGAACATCGACGGCTACTTCAAGATCTA
CAGCAAGCACACCCCTATCAACCTCggcCGGGATCTGCCTCAGGGCTTCTCTGCTC
TGGAACCCCTGGTGGATCTGCCCATCGGCATCAACATCACCCGGTTTCAGACACT
GCTGGCCCTGCACAGAAGCTACCTGACACCTGGCGATAGCAGCAGCGGATGGAC
AGCTGGTGCCGCCGCTTACTATGTGGGCTACCTGCAGCCTAGAACCTTTCTGCTG
AAGTACAACGAGAACGGCACCATCACCGACGCCGTGGATTGTGCTCTGGATCCT
CTGAGCGAGACAAAGTGCACCCTGAAGTCCTTCACCGTGGAgAAGGGCATCTAC
CAGACCAGCAACTTCCGGGTGCAGCCCACCGAATCCATCGTGCGGTTCCCCAATA
TCACCAATCTGTGCCCCTTCgacGAGGTGTTCAATGCCACCAGATTCGCCTCTGTG
TACGCCTGGAACCGGAAGCGGATCAGCAATTGCGTGGCCGACTACTCCGTGCTG
TACAACttcGCCcctTTCttcgccTTCAAGTGCTACGGCGTGTCCCCTACCAAGCTGAAC
GACCTGTGCTTCACAAACGTGTACGCCGACAGCTTCGTGATCCGGGGAaacGAAG
TGagcCAGATTGCCCCTGGACAGACAGGCaacATCGCCGACTACAACTACAAGCTG
CCCGACGACTTCACCGGCTGTGTGATTGCCTGGAACAGCAACaagCTGGACTCCAA
AGTCGGCGGCAACTACAATTACcagTACCGGCTGTTCCGGAAGTCCAATCTGAAG
CCCTTCGAGCGGGACATCTCCACCGAGATCTATCAGGCCGGCaacaagCCTTGTAAC
GGCGTGgccGGCTTCAACTGCTACTTCCCACTGcggTCCTACGGCTTTcggCCCACAta
cGGCGTGGGCcacCAGCCCTACAGAGTGGTGGTGCTGAGCTTCGAACTGCTGCATG
CCCCTGCCACAGTGTGCGGCCCTAAGAAgAGCACCAATCTCGTGAAGAACAAAT
GCGTGAACTTCAACTTCAACGGCCTGACCGGCACCGGCGTGCTGACAGAGAGCA
ACAAGAAGTTCCTGCCATTCCAGCAGTTTGGCCGGGATATCGCCGATACCACAG
ACGCCGTTAGAGATCCCCAGACACTGGAAATCCTGGACATCACCCCTTGCAGCTT
CGGCGGAGTGTCTGTGATCACCCCTGGCACCAACACCAGCAATCAGGTGGCAGT
GCTGTACCAGggcGTGAACTGTACCGAAGTGCCCGTGGCCATTCACGCCGATCAG
CTGACACCTACATGGCGGGTGTACTCCACCGGCAGCAATGTGTTTCAGACCAGA
GCCGGCTGTCTGATCGGAGCCGAGtacGTGAACAATAGCTACGAGTGCGACATCC
CCATCGGCGCTGGCATCTGTGCCAGCTACCAGACACAGACAaagAGCcacGCCTCT
GTGGCCAGCCAGAGCATCATTGCCTACACAATGTCTCTGGGCGCCGAGAACctgGT
GGCCTACTCCAACAACTCTATCGCTATCCCCACCAACTTCACCATCAGCGTGACC
ACAGAGATCCTGCCTGTGTCCATGACCAAGACCAGCGTGGACTGCACCATGTAC
ATCTGCGGCGATTCCACCGAGTGCTCCAACCTGCTGCTGCAGTACGGCAGCTTCT
GCACCCAGCTGaagAGAGCCCTGACAGGGATCGCCGTGGAACAGGACAAGAACA
CCCAAGAGGTGTTCGCCCAAGTGAAGCAGATCTACAAGACCCCTCCTATCAAGtac
TTCGGCGGCTTCAATTTCAGCCAGATTCTGCCCGATCCTAGCAAGCCCAGCAAGC
GGAGCcctATCGAGGACCTGCTGTTCAACAAAGTGACACTGGCCGACGCCGGCTTC
ATCAAGCAGTATGGCGATTGTCTGGGCGACATTGCCGCCAGGGATCTGATTTGCG
CCCAGAAGTTTAACGGACTGACAGTGCTGCCTCCTCTGCTGACCGATGAGATGAT
CGCCCAGTACACATCTGCCCTGCTGGCCGGCACAATCACAAGCGGCTGGACATTT
GGAGCTGGCcctGCTCTGCAGATCCCCTTTccaATGCAGATGGCCTACCGGTTCAAC
GGCATCGGAGTGACCCAGAATGTGCTGTACGAGAACCAGAAGCTGATCGCCAAC
CAGTTCAACAGCGCCATCGGCAAGATCCAGGACAGCCTGAGCAGCACAcccAGCG
CCCTGGGAAAGCTGCAGGACGTGGTCAACcacAATGCCCAGGCACTGAACACCCT
GGTCAAGCAGCTGTCCTCCaagTTCGGCGCCATCAGCTCTGTGCTGAACGATATCC
TGAGCAGACTGGACccccctGAAGCCGAGGTGCAGATCGACAGACTGATCACCGGA
AGGCTGCAGTCCCTGCAGACCTACGTTACCCAGCAGCTGATCAGAGCCGCCGAG
ATTAGAGCCTCTGCCAATCTGGCCGCCACCAAGATGTCTGAGTGTGTGCTGGGCC
AGAGCAAGAGAGTGGACTTTTGCGGCAAGGGCTACCACCTGATGAGCTTCCCTC
AGTCTGCCCCTCACGGCGTGGTGTTTCTGCACGTGACATACGTGCCCGCTCAAGA
GAAGAATTTCACCACCGCTCCAGCCATCTGCCACGACGGCAAAGCCCACTTTCCT
AGAGAAGGCGTGTTCGTGTCCAACGGCACCCATTGGTTCGTGACCCAGCGGAAC
TTCTACGAGCCCCAGATCATCACCACCGACAACACCTTCGTGTCTGGCAACTGCG
ACGTCGTGATCGGCATTGTGAACAATACCGTGTACGACCCTCTGCAGCCCGAGCT
GGACAGCTTCAAAGAGGAACTGGATAAGTACTTTAAGAACCACACAAGCCCCGA
CGTGGACCTGGGCGATATCAGCGGAATCAATGCCAGCGTCGTGAACATCCAGAA
AGAGATCGACCGGCTGAACGAGGTGGCCAAGAATCTGAACGAGAGCCTGATCGA
CCTGCAAGAACTGGGGAAGTACGAGCAGTACATCAAGTGGCCC
Amino Acid Sequence of BA.2.12.1 (Signal peptide is in bold and underlined; Spike
protein ectodomain underlined): SEQ ID NO: 51
MFVFLVLLPLVSSQCVNLITRTQSYTNSFTRGVYYPDKVFRSSVLHSTQDLFLPFFSN
VTWFHAIHVSGTNGTKRFDNPVLPFNDGVYFASTEKSNIIRGWIFGTTLDSKTQSLLI
VNNATNVVIKVCEFQFCNDPFLDVYYHKNNKSWMESEFRVYSSANNCTFEYVSQPF
LMDLEGKQGNFKNLREFVFKNIDGYFKIYSKHTPINLGRDLPQGFSALEPLVDLPIGIN
ITRFQTLLALHRSYLTPGDSSSGWTAGAAAYYVGYLQPRTFLLKYNENGTITDAVDC
ALDPLSETKCTLKSFTVEKGIYQTSNFRVQPTESIVRFPNITNLCPFDEVFNATRFASV
YAWNRKRISNCVADYSVLYNFAPFFAFKCYGVSPTKLNDLCFTNVYADSFVIRGNE
VSQIAPGQTGNIADYNYKLPDDFTGCVIAWNSNKLDSKVGGNYNYQYRLFRKSNLK
PFERDISTEIYQAGNKPCNGVAGFNCYFPLRSYGFRPTYGVGHQPYRVVVLSFELLH
APATVCGPKKSTNLVKNKCVNFNFNGLTGTGVLTESNKKFLPFQQFGRDIADTTDA
VRDPQTLEILDITPCSFGGVSVITPGTNTSNQVAVLYQGVNCTEVPVAIHADQLTPTW
RVYSTGSNVFQTRAGCLIGAEYVNNSYECDIPIGAGICASYQTQTKSHASVASQSIIAY
TMSLGAENLVAYSNNSIAIPTNFTISVTTEILPVSMTKTSVDCTMYICGDSTECSNLLL
QYGSFCTQLKRALTGIAVEQDKNTQEVFAQVKQIYKTPPIKYFGGFNFSQILPDPSKP
SKRSPIEDLLFNKVTLADAGFIKQYGDCLGDIAARDLICAQKFNGLTVLPPLLTDEMI
AQYTSALLAGTITSGWTFGAGPALQIPFPMQMAYRENGIGVTQNVLYENQKLIANQF
NSAIGKIQDSLSSTPSALGKLQDVVNHNAQALNTLVKQLSSKFGAISSVLNDILSRLD
PPEAEVQIDRLITGRLQSLQTYVTQQLIRAAEIRASANLAATKMSECVLGQSKRVDFC
GKGYHLMSFPQSAPHGVVFLHVTYVPAQEKNFTTAPAICHDGKAHFPREGVFVSNG
THWFVTQRNFYEPQIITTDNTFVSGNCDVVIGIVNNTVYDPLQPELDSFKEELDKYFK
NHTSPDVDLGDISGINASVVNIQKEIDRLNEVAKNLNESLIDLQELGKYEQYIKWP
Nucleotide Sequence of BA.2.12.1 (Spike protein ectodomain without signal
peptide): SEQ ID NO: 52
CAGTGTGTGAACCTGatcACAAGAACCCAGagcTACACCAACAGCTTTACCAGAGG
CGTGTACTACCCCGACAAGGTGTTCAGATCCAGCGTGCTGCACTCTACCCAGGAC
CTGTTCCTGCCTTTCTTCAGCAACGTGACCTGGTTCCACGCCATCCACGTGTCCGG
CACCAATGGCACCAAGAGATTCGACAACCCCGTGCTGCCCTTCAACGACGGGGT
GTACTTTGCCAGCACCGAGAAGTCCAACATCATCAGAGGCTGGATCTTCGGCACC
ACACTGGACAGCAAGACCCAGAGCCTGCTGATCGTGAACAACGCCACCAACGTG
GTCATCAAAGTGTGCGAGTTCCAGTTCTGCAACGACCCCTTCCTGgacGTCTACTA
TCACAAGAACAACAAGAGCTGGATGGAAAGCGAGTTCCGGGTGTACAGCAGCGC
CAACAACTGCACCTTCGAGTACGTGTCCCAGCCTTTCCTGATGGACCTGGAAGGC
AAGCAGGGCAACTTCAAGAACCTGCGCGAGTTCGTGTTCAAGAACATCGACGGC
TACTTCAAGATCTACAGCAAGCACACCCCTATCAACCTCggcCGGGATCTGCCTCA
GGGCTTCTCTGCTCTGGAACCCCTGGTGGATCTGCCCATCGGCATCAACATCACC
CGGTTTCAGACACTGCTGGCCCTGCACAGAAGCTACCTGACACCTGGCGATAGC
AGCAGCGGATGGACAGCTGGTGCCGCCGCTTACTATGTGGGCTACCTGCAGCCT
AGAACCTTTCTGCTGAAGTACAACGAGAACGGCACCATCACCGACGCCGTGGAT
TGTGCTCTGGATCCTCTGAGCGAGACAAAGTGCACCCTGAAGTCCTTCACCGTGG
AgAAGGGCATCTACCAGACCAGCAACTTCCGGGTGCAGCCCACCGAATCCATCGT
GCGGTTCCCCAATATCACCAATCTGTGCCCCTTCgacGAGGTGTTCAATGCCACCA
GATTCGCCTCTGTGTACGCCTGGAACCGGAAGCGGATCAGCAATTGCGTGGCCG
ACTACTCCGTGCTGTACAACttcGCCcctTTCttegccTTCAAGTGCTACGGCGTGTCCCC
TACCAAGCTGAACGACCTGTGCTTCACAAACGTGTACGCCGACAGCTTCGTGATC
CGGGGAaacGAAGTGagcCAGATTGCCCCTGGACAGACAGGCaacATCGCCGACTAC
AACTACAAGCTGCCCGACGACTTCACCGGCTGTGTGATTGCCTGGAACAGCAACa
agCTGGACTCCAAAGTCGGCGGCAACTACAATTACcagTACCGGCTGTTCCGGAAG
TCCAATCTGAAGCCCTTCGAGCGGGACATCTCCACCGAGATCTATCAGGCCGGCa
acaagCCTTGTAACGGCGTGgccGGCTTCAACTGCTACTTCCCACTGcggTCCTACGGC
TTTcggCCCACAtacGGCGTGGGCcacCAGCCCTACAGAGTGGTGGTGCTGAGCTTCG
AACTGCTGCATGCCCCTGCCACAGTGTGCGGCCCTAAGAAgAGCACCAATCTCGT
GAAGAACAAATGCGTGAACTTCAACTTCAACGGCCTGACCGGCACCGGCGTGCT
GACAGAGAGCAACAAGAAGTTCCTGCCATTCCAGCAGTTTGGCCGGGATATCGC
CGATACCACAGACGCCGTTAGAGATCCCCAGACACTGGAAATCCTGGACATCAC
CCCTTGCAGCTTCGGCGGAGTGTCTGTGATCACCCCTGGCACCAACACCAGCAAT
CAGGTGGCAGTGCTGTACCAGggcGTGAACTGTACCGAAGTGCCCGTGGCCATTC
ACGCCGATCAGCTGACACCTACATGGCGGGTGTACTCCACCGGCAGCAATGTGTT
TCAGACCAGAGCCGGCTGTCTGATCGGAGCCGAGtacGTGAACAATAGCTACGAG
TGCGACATCCCCATCGGCGCTGGCATCTGTGCCAGCTACCAGACACAGACAaagA
GCcacGCCTCTGTGGCCAGCCAGAGCATCATTGCCTACACAATGTCTCTGGGCGCC
GAGAACctgGTGGCCTACTCCAACAACTCTATCGCTATCCCCACCAACTTCACCAT
CAGCGTGACCACAGAGATCCTGCCTGTGTCCATGACCAAGACCAGCGTGGACTG
CACCATGTACATCTGCGGCGATTCCACCGAGTGCTCCAACCTGCTGCTGCAGTAC
GGCAGCTTCTGCACCCAGCTGaagAGAGCCCTGACAGGGATCGCCGTGGAACAGG
ACAAGAACACCCAAGAGGTGTTCGCCCAAGTGAAGCAGATCTACAAGACCCCTC
CTATCAAGtacTTCGGCGGCTTCAATTTCAGCCAGATTCTGCCCGATCCTAGCAAG
CCCAGCAAGCGGAGCcctATCGAGGACCTGCTGTTCAACAAAGTGACACTGGCCG
ACGCCGGCTTCATCAAGCAGTATGGCGATTGTCTGGGCGACATTGCCGCCAGGG
ATCTGATTTGCGCCCAGAAGTTTAACGGACTGACAGTGCTGCCTCCTCTGCTGAC
CGATGAGATGATCGCCCAGTACACATCTGCCCTGCTGGCCGGCACAATCACAAG
CGGCTGGACATTTGGAGCTGGCcctGCTCTGCAGATCCCCTTTccaATGCAGATGGC
CTACCGGTTCAACGGCATCGGAGTGACCCAGAATGTGCTGTACGAGAACCAGAA
GCTGATCGCCAACCAGTTCAACAGCGCCATCGGCAAGATCCAGGACAGCCTGAG
CAGCACAcccAGCGCCCTGGGAAAGCTGCAGGACGTGGTCAACcacAATGCCCAGG
CACTGAACACCCTGGTCAAGCAGCTGTCCTCCaagTTCGGCGCCATCAGCTCTGTG
CTGAACGATATCCTGAGCAGACTGGACccccctGAAGCCGAGGTGCAGATCGACAG
ACTGATCACCGGAAGGCTGCAGTCCCTGCAGACCTACGTTACCCAGCAGCTGATC
AGAGCCGCCGAGATTAGAGCCTCTGCCAATCTGGCCGCCACCAAGATGTCTGAG
TGTGTGCTGGGCCAGAGCAAGAGAGTGGACTTTTGCGGCAAGGGCTACCACCTG
ATGAGCTTCCCTCAGTCTGCCCCTCACGGCGTGGTGTTTCTGCACGTGACATACG
TGCCCGCTCAAGAGAAGAATTTCACCACCGCTCCAGCCATCTGCCACGACGGCA
AAGCCCACTTTCCTAGAGAAGGCGTGTTCGTGTCCAACGGCACCCATTGGTTCGT
GACCCAGCGGAACTTCTACGAGCCCCAGATCATCACCACCGACAACACCTTCGT
GTCTGGCAACTGCGACGTCGTGATCGGCATTGTGAACAATACCGTGTACGACCCT
CTGCAGCCCGAGCTGGACAGCTTCAAAGAGGAACTGGATAAGTACTTTAAGAAC
CACACAAGCCCCGACGTGGACCTGGGCGATATCAGCGGAATCAATGCCAGCGTC
GTGAACATCCAGAAAGAGATCGACCGGCTGAACGAGGTGGCCAAGAATCTGAAC
GAGAGCCTGATCGACCTGCAAGAACTGGGGAAGTACGAGCAGTACATCAAGTGG
CCC
Amino Acid Sequence of BA.2.12.1 (Spike protein ectodomain without signal
peptide): SEQ ID NO: 53
QCVNLITRTQSYTNSFTRGVYYPDKVFRSSVLHSTQDLFLPFFSNVTWFHAIHVSGTN
GTKRFDNPVLPFNDGVYFASTEKSNIIRGWIFGTTLDSKTQSLLIVNNATNVVIKVCEF
QFCNDPFLDVYYHKNNKSWMESEFRVYSSANNCTFEYVSQPFLMDLEGKQGNFKN
LREFVFKNIDGYFKIYSKHTPINLGRDLPQGFSALEPLVDLPIGINITRFQTLLALHRSY
LTPGDSSSGWTAGAAAYYVGYLQPRTFLLKYNENGTITDAVDCALDPLSETKCTLKS
FTVEKGIYQTSNFRVQPTESIVRFPNITNLCPFDEVFNATRFASVYAWNRKRISNCVA
DYSVLYNFAPFFAFKCYGVSPTKLNDLCFTNVYADSFVIRGNEVSQIAPGQTGNIAD
YNYKLPDDFTGCVIAWNSNKLDSKVGGNYNYQYRLFRKSNLKPFERDISTEIYQAG
NKPCNGVAGFNCYFPLRSYGFRPTYGVGHQPYRVVVLSFELLHAPATVCGPKKSTN
LVKNKCVNFNFNGLTGTGVLTESNKKFLPFQQFGRDIADTTDAVRDPQTLEILDITPC
SFGGVSVITPGTNTSNQVAVLYQGVNCTEVPVAIHADQLTPTWRVYSTGSNVFQTRA
GCLIGAEYVNNSYECDIPIGAGICASYQTQTKSHASVASQSIIAYTMSLGAENLVAYS
NNSIAIPTNFTISVTTEILPVSMTKTSVDCTMYICGDSTECSNLLLQYGSFCTQLKRAL
TGIAVEQDKNTQEVFAQVKQIYKTPPIKYFGGFNFSQILPDPSKPSKRSPIEDLLFNKV
TLADAGFIKQYGDCLGDIAARDLICAQKFNGLTVLPPLLTDEMIAQYTSALLAGTITS
GWTFGAGPALQIPFPMQMAYRFNGIGVTQNVLYENQKLIANQFNSAIGKIQDSLSSTP
SALGKLQDVVNHNAQALNTLVKQLSSKFGAISSVLNDILSRLDPPEAEVQIDRLITGR
LQSLQTYVTQQLIRAAEIRASANLAATKMSECVLGQSKRVDFCGKGYHLMSFPQSAP
HGVVFLHVTYVPAQEKNFTTAPAICHDGKAHFPREGVFVSNGTHWFVTQRNFYEPQ
IITTDNTFVSGNCDVVIGIVNNTVYDPLQPELDSFKEELDKYFKNHTSPDVDLGDISGI
NASVVNIQKEIDRLNEVAKNLNESLIDLQELGKYEQYIKWP
Nucleotide Sequence of BA.2.12.1 S371 S373 S375 (Signal peptide is in bold and
underlined; GS linker is in italics; TM and CT From F is in bold and double
underlined; Spike ectodomain is underlined; S371 S373 S375 is in bold, underlined
and italics): SEQ ID NO: 54
ATGTTCGTGTTTCTGGTGCTGCTGCCTCTGGTGTCCAGCCAGTGTGTGAACCT
GatcACAAGAACCCAGagcTACACCAACAGCTTTACCAGAGGCGTGTACTACCCCG
ACAAGGTGTTCAGATCCAGCGTGCTGCACTCTACCCAGGACCTGTTCCTGCCTTT
CTTCAGCAACGTGACCTGGTTCCACGCCATCCACGTGTCCGGCACCAATGGCACC
AAGAGATTCGACAACCCCGTGCTGCCCTTCAACGACGGGGTGTACTTTGCCAGCA
CCGAGAAGTCCAACATCATCAGAGGCTGGATCTTCGGCACCACACTGGACAGCA
AGACCCAGAGCCTGCTGATCGTGAACAACGCCACCAACGTGGTCATCAAAGTGT
GCGAGTTCCAGTTCTGCAACGACCCCTTCCTGgacGTCTACTATCACAAGAACAAC
AAGAGCTGGATGGAAAGCGAGTTCCGGGTGTACAGCAGCGCCAACAACTGCACC
TTCGAGTACGTGTCCCAGCCTTTCCTGATGGACCTGGAAGGCAAGCAGGGCAACT
TCAAGAACCTGCGCGAGTTCGTGTTCAAGAACATCGACGGCTACTTCAAGATCTA
CAGCAAGCACACCCCTATCAACCTCggcCGGGATCTGCCTCAGGGCTTCTCTGCTC
TGGAACCCCTGGTGGATCTGCCCATCGGCATCAACATCACCCGGTTTCAGACACT
GCTGGCCCTGCACAGAAGCTACCTGACACCTGGCGATAGCAGCAGCGGATGGAC
AGCTGGTGCCGCCGCTTACTATGTGGGCTACCTGCAGCCTAGAACCTTTCTGCTG
AAGTACAACGAGAACGGCACCATCACCGACGCCGTGGATTGTGCTCTGGATCCT
CTGAGCGAGACAAAGTGCACCCTGAAGTCCTTCACCGTGGAgAAGGGCATCTAC
CAGACCAGCAACTTCCGGGTGCAGCCCACCGAATCCATCGTGCGGTTCCCCAATA
TCACCAATCTGTGCCCCTTCgacGAGGTGTTCAATGCCACCAGATTCGCCTCTGTG
TACGCCTGGAACCGGAAGCGGATCAGCAATTGCGTGGCCGACTACTCCGTGCTG
TACAACtccGCCagcTTCagcgccTTCAAGTGCTACGGCGTGTCCCCTACCAAGCTGAA
CGACCTGTGCTTCACAAACGTGTACGCCGACAGCTTCGTGATCCGGGGAaacGAA
GTGagcCAGATTGCCCCTGGACAGACAGGCaacATCGCCGACTACAACTACAAGCT
GCCCGACGACTTCACCGGCTGTGTGATTGCCTGGAACAGCAACaagCTGGACTCCA
AAGTCGGCGGCAACTACAATTACcagTACCGGCTGTTCCGGAAGTCCAATCTGAA
GCCCTTCGAGCGGGACATCTCCACCGAGATCTATCAGGCCGGCaacaagCCTTGTAA
CGGCGTGgccGGCTTCAACTGCTACTTCCCACTGcggTCCTACGGCTTTcggCCCACAt
acGGCGTGGGCcacCAGCCCTACAGAGTGGTGGTGCTGAGCTTCGAACTGCTGCAT
GCCCCTGCCACAGTGTGCGGCCCTAAGAAgAGCACCAATCTCGTGAAGAACAAA
TGCGTGAACTTCAACTTCAACGGCCTGACCGGCACCGGCGTGCTGACAGAGAGC
AACAAGAAGTTCCTGCCATTCCAGCAGTTTGGCCGGGATATCGCCGATACCACA
GACGCCGTTAGAGATCCCCAGACACTGGAAATCCTGGACATCACCCCTTGCAGCT
TCGGCGGAGTGTCTGTGATCACCCCTGGCACCAACACCAGCAATCAGGTGGCAG
TGCTGTACCAGggcGTGAACTGTACCGAAGTGCCCGTGGCCATTCACGCCGATCA
GCTGACACCTACATGGCGGGTGTACTCCACCGGCAGCAATGTGTTTCAGACCAG
AGCCGGCTGTCTGATCGGAGCCGAGtacGTGAACAATAGCTACGAGTGCGACATC
CCCATCGGCGCTGGCATCTGTGCCAGCTACCAGACACAGACAaagAGCcacGCCTCT
GTGGCCAGCCAGAGCATCATTGCCTACACAATGTCTCTGGGCGCCGAGAACctgGT
GGCCTACTCCAACAACTCTATCGCTATCCCCACCAACTTCACCATCAGCGTGACC
ACAGAGATCCTGCCTGTGTCCATGACCAAGACCAGCGTGGACTGCACCATGTAC
ATCTGCGGCGATTCCACCGAGTGCTCCAACCTGCTGCTGCAGTACGGCAGCTTCT
GCACCCAGCTGaagAGAGCCCTGACAGGGATCGCCGTGGAACAGGACAAGAACA
CCCAAGAGGTGTTCGCCCAAGTGAAGCAGATCTACAAGACCCCTCCTATCAAGtac
TTCGGCGGCTTCAATTTCAGCCAGATTCTGCCCGATCCTAGCAAGCCCAGCAAGC
GGAGCcctATCGAGGACCTGCTGTTCAACAAAGTGACACTGGCCGACGCCGGCTTC
ATCAAGCAGTATGGCGATTGTCTGGGCGACATTGCCGCCAGGGATCTGATTTGCG
CCCAGAAGTTTAACGGACTGACAGTGCTGCCTCCTCTGCTGACCGATGAGATGAT
CGCCCAGTACACATCTGCCCTGCTGGCCGGCACAATCACAAGCGGCTGGACATTT
GGAGCTGGCcctGCTCTGCAGATCCCCTTTccaATGCAGATGGCCTACCGGTTCAAC
GGCATCGGAGTGACCCAGAATGTGCTGTACGAGAACCAGAAGCTGATCGCCAAC
CAGTTCAACAGCGCCATCGGCAAGATCCAGGACAGCCTGAGCAGCACAcccAGCG
CCCTGGGAAAGCTGCAGGACGTGGTCAACcacAATGCCCAGGCACTGAACACCCT
GGTCAAGCAGCTGTCCTCCaagTTCGGCGCCATCAGCTCTGTGCTGAACGATATCC
TGAGCAGACTGGACccccctGAAGCCGAGGTGCAGATCGACAGACTGATCACCGGA
AGGCTGCAGTCCCTGCAGACCTACGTTACCCAGCAGCTGATCAGAGCCGCCGAG
ATTAGAGCCTCTGCCAATCTGGCCGCCACCAAGATGTCTGAGTGTGTGCTGGGCC
AGAGCAAGAGAGTGGACTTTTGCGGCAAGGGCTACCACCTGATGAGCTTCCCTC
AGTCTGCCCCTCACGGCGTGGTGTTTCTGCACGTGACATACGTGCCCGCTCAAGA
GAAGAATTTCACCACCGCTCCAGCCATCTGCCACGACGGCAAAGCCCACTTTCCT
AGAGAAGGCGTGTTCGTGTCCAACGGCACCCATTGGTTCGTGACCCAGCGGAAC
TTCTACGAGCCCCAGATCATCACCACCGACAACACCTTCGTGTCTGGCAACTGCG
ACGTCGTGATCGGCATTGTGAACAATACCGTGTACGACCCTCTGCAGCCCGAGCT
GGACAGCTTCAAAGAGGAACTGGATAAGTACTTTAAGAACCACACAAGCCCCGA
CGTGGACCTGGGCGATATCAGCGGAATCAATGCCAGCGTCGTGAACATCCAGAA
AGAGATCGACCGGCTGAACGAGGTGGCCAAGAATCTGAACGAGAGCCTGATCGA
CCTGCAAGAACTGGGGAAGTACGAGCAGTACATCAAGTGGCCCggcggaggtgggtcgC
TCATAACATACATCGTCCTGACTATAATCAGCTTGGTATTTGGTATTTTGTCT
TTGATTCTTGCATGCTATTTGATGTATAAACAGAAAGCTCAGCAGAAGACTC
TCCTGTGGCTCGGTAACAACACACTCGACCAGATGAGAGCAACTACAAAGA
TGTGA
Amino Acid Sequence of BA.2.12.1 S371 S373 S375 (Signal peptide is in bold and
underlined; GS linker is in italics; TM and CT From F is in bold and double
underlined; Spike ectodomain is underlined; S371 S373 S375 is in bold, underlined
and italics): SEQ ID NO: 55
MFVFLVLLPLVSSQCVNLITRTQSYTNSFTRGVYYPDKVFRSSVLHSTQDLFLPFFSN
VTWFHAIHVSGTNGTKRFDNPVLPFNDGVYFASTEKSNIIRGWIFGTTLDSKTQSLLI
VNNATNVVIKVCEFQFCNDPFLDVYYHKNNKSWMESEFRVYSSANNCTFEYVSQPF
LMDLEGKQGNFKNLREFVFKNIDGYFKIYSKHTPINLGRDLPQGFSALEPLVDLPIGIN
ITRFQTLLALHRSYLTPGDSSSGWTAGAAAYYVGYLQPRTELLKYNENGTITDAVDC
ALDPLSETKCTLKSFTVEKGIYQTSNFRVQPTESIVRFPNITNLCPFDEVFNATRFASV
YAWNRKRISNCVADYSVLYNSASFSAFKCYGVSPTKLNDLCFTNVYADSFVIRGNE
VSQIAPGQTGNIADYNYKLPDDFTGCVIAWNSNKLDSKVGGNYNYQYRLFRKSNLK
PFERDISTEIYQAGNKPCNGVAGFNCYFPLRSYGFRPTYGVGHQPYRVVVLSFELLH
APATVCGPKKSTNLVKNKCVNFNFNGLTGTGVLTESNKKFLPFQQFGRDIADTTDA
VRDPQTLEILDITPCSFGGVSVITPGTNTSNQVAVLYQGVNCTEVPVAIHADQLTPTW
RVYSTGSNVFQTRAGCLIGAEYVNNSYECDIPIGAGICASYQTQTKSHASVASQSIIAY
TMSLGAENLVAYSNNSIAIPTNFTISVTTEILPVSMTKTSVDCTMYICGDSTECSNLLL
QYGSFCTQLKRALTGIAVEQDKNTQEVFAQVKQIYKTPPIKYFGGFNFSQILPDPSKP
SKRSPIEDLLFNKVTLADAGFIKQYGDCLGDIAARDLICAQKFNGLTVLPPLLTDEMI
AQYTSALLAGTITSGWTFGAGPALQIPFPMQMAYRENGIGVTQNVLYENQKLIANQF
NSAIGKIQDSLSSTPSALGKLQDVVNHNAQALNTLVKQLSSKFGAISSVLNDILSRLD
PPEAEVQIDRLITGRLQSLQTYVTQQLIRAAEIRASANLAATKMSECVLGQSKRVDFC
GKGYHLMSFPQSAPHGVVFLHVTYVPAQEKNFTTAPAICHDGKAHFPREGVFVSNG
THWFVTQRNFYEPQIITTDNTFVSGNCDVVIGIVNNTVYDPLQPELDSFKEELDKYFK
NHTSPDVDLGDISGINASVVNIQKEIDRLNEVAKNLNESLIDLQELGKYEQYIKWPGG
GGSLITYIVLTIISLVFGILSLILACYLMYKQKAQQKTLLWLGNNTLDQMRATTK
M*
Nucleotide Sequence of BA.2.12.1 S371 S373 S375 (Signal peptide is in bold and
underlined; Spike ectodomain is underlined; S371 S373 S375 is in bold, underlined
and italics): SEQ ID NO: 56
ATGTTCGTGTTTCTGGTGCTGCTGCCTCTGGTGTCCAGCCAGTGTGTGAACCT
GatcACAAGAACCCAGagcTACACCAACAGCTTTACCAGAGGCGTGTACTACCCCG
ACAAGGTGTTCAGATCCAGCGTGCTGCACTCTACCCAGGACCTGTTCCTGCCTTT
CTTCAGCAACGTGACCTGGTTCCACGCCATCCACGTGTCCGGCACCAATGGCACC
AAGAGATTCGACAACCCCGTGCTGCCCTTCAACGACGGGGTGTACTTTGCCAGCA
CCGAGAAGTCCAACATCATCAGAGGCTGGATCTTCGGCACCACACTGGACAGCA
AGACCCAGAGCCTGCTGATCGTGAACAACGCCACCAACGTGGTCATCAAAGTGT
GCGAGTTCCAGTTCTGCAACGACCCCTTCCTGgacGTCTACTATCACAAGAACAAC
AAGAGCTGGATGGAAAGCGAGTTCCGGGTGTACAGCAGCGCCAACAACTGCACC
TTCGAGTACGTGTCCCAGCCTTTCCTGATGGACCTGGAAGGCAAGCAGGGCAACT
TCAAGAACCTGCGCGAGTTCGTGTTCAAGAACATCGACGGCTACTTCAAGATCTA
CAGCAAGCACACCCCTATCAACCTCggcCGGGATCTGCCTCAGGGCTTCTCTGCTC
TGGAACCCCTGGTGGATCTGCCCATCGGCATCAACATCACCCGGTTTCAGACACT
GCTGGCCCTGCACAGAAGCTACCTGACACCTGGCGATAGCAGCAGCGGATGGAC
AGCTGGTGCCGCCGCTTACTATGTGGGCTACCTGCAGCCTAGAACCTTTCTGCTG
AAGTACAACGAGAACGGCACCATCACCGACGCCGTGGATTGTGCTCTGGATCCT
CTGAGCGAGACAAAGTGCACCCTGAAGTCCTTCACCGTGGAgAAGGGCATCTAC
CAGACCAGCAACTTCCGGGTGCAGCCCACCGAATCCATCGTGCGGTTCCCCAATA
TCACCAATCTGTGCCCCTTCgacGAGGTGTTCAATGCCACCAGATTCGCCTCTGTG
TACGCCTGGAACCGGAAGCGGATCAGCAATTGCGTGGCCGACTACTCCGTGCTG
TACAACtccGCCagcTTCagcgccTTCAAGTGCTACGGCGTGTCCCCTACCAAGCTGAA
CGACCTGTGCTTCACAAACGTGTACGCCGACAGCTTCGTGATCCGGGGAaacGAA
GTGagcCAGATTGCCCCTGGACAGACAGGCaacATCGCCGACTACAACTACAAGCT
GCCCGACGACTTCACCGGCTGTGTGATTGCCTGGAACAGCAACaagCTGGACTCCA
AAGTCGGCGGCAACTACAATTACcagTACCGGCTGTTCCGGAAGTCCAATCTGAA
GCCCTTCGAGCGGGACATCTCCACCGAGATCTATCAGGCCGGCaacaagCCTTGTAA
CGGCGTGgccGGCTTCAACTGCTACTTCCCACTGcggTCCTACGGCTTTcggCCCACAt
acGGCGTGGGCcacCAGCCCTACAGAGTGGTGGTGCTGAGCTTCGAACTGCTGCAT
GCCCCTGCCACAGTGTGCGGCCCTAAGAAgAGCACCAATCTCGTGAAGAACAAA
TGCGTGAACTTCAACTTCAACGGCCTGACCGGCACCGGCGTGCTGACAGAGAGC
AACAAGAAGTTCCTGCCATTCCAGCAGTTTGGCCGGGATATCGCCGATACCACA
GACGCCGTTAGAGATCCCCAGACACTGGAAATCCTGGACATCACCCCTTGCAGCT
TCGGCGGAGTGTCTGTGATCACCCCTGGCACCAACACCAGCAATCAGGTGGCAG
TGCTGTACCAGggcGTGAACTGTACCGAAGTGCCCGTGGCCATTCACGCCGATCA
GCTGACACCTACATGGCGGGTGTACTCCACCGGCAGCAATGTGTTTCAGACCAG
AGCCGGCTGTCTGATCGGAGCCGAGtacGTGAACAATAGCTACGAGTGCGACATC
CCCATCGGCGCTGGCATCTGTGCCAGCTACCAGACACAGACAaagAGCcacGCCTCT
GTGGCCAGCCAGAGCATCATTGCCTACACAATGTCTCTGGGCGCCGAGAACctgGT
GGCCTACTCCAACAACTCTATCGCTATCCCCACCAACTTCACCATCAGCGTGACC
ACAGAGATCCTGCCTGTGTCCATGACCAAGACCAGCGTGGACTGCACCATGTAC
ATCTGCGGCGATTCCACCGAGTGCTCCAACCTGCTGCTGCAGTACGGCAGCTTCT
GCACCCAGCTGaagAGAGCCCTGACAGGGATCGCCGTGGAACAGGACAAGAACA
CCCAAGAGGTGTTCGCCCAAGTGAAGCAGATCTACAAGACCCCTCCTATCAAGtac
TTCGGCGGCTTCAATTTCAGCCAGATTCTGCCCGATCCTAGCAAGCCCAGCAAGC
GGAGCcctATCGAGGACCTGCTGTTCAACAAAGTGACACTGGCCGACGCCGGCTTC
ATCAAGCAGTATGGCGATTGTCTGGGCGACATTGCCGCCAGGGATCTGATTTGCG
CCCAGAAGTTTAACGGACTGACAGTGCTGCCTCCTCTGCTGACCGATGAGATGAT
CGCCCAGTACACATCTGCCCTGCTGGCCGGCACAATCACAAGCGGCTGGACATTT
GGAGCTGGCcctGCTCTGCAGATCCCCTTTccaATGCAGATGGCCTACCGGTTCAAC
GGCATCGGAGTGACCCAGAATGTGCTGTACGAGAACCAGAAGCTGATCGCCAAC
CAGTTCAACAGCGCCATCGGCAAGATCCAGGACAGCCTGAGCAGCACAcccAGCG
CCCTGGGAAAGCTGCAGGACGTGGTCAACcacAATGCCCAGGCACTGAACACCCT
GGTCAAGCAGCTGTCCTCCaagTTCGGCGCCATCAGCTCTGTGCTGAACGATATCC
TGAGCAGACTGGACccccctGAAGCCGAGGTGCAGATCGACAGACTGATCACCGGA
AGGCTGCAGTCCCTGCAGACCTACGTTACCCAGCAGCTGATCAGAGCCGCCGAG
ATTAGAGCCTCTGCCAATCTGGCCGCCACCAAGATGTCTGAGTGTGTGCTGGGCC
AGAGCAAGAGAGTGGACTTTTGCGGCAAGGGCTACCACCTGATGAGCTTCCCTC
AGTCTGCCCCTCACGGCGTGGTGTTTCTGCACGTGACATACGTGCCCGCTCAAGA
GAAGAATTTCACCACCGCTCCAGCCATCTGCCACGACGGCAAAGCCCACTTTCCT
AGAGAAGGCGTGTTCGTGTCCAACGGCACCCATTGGTTCGTGACCCAGCGGAAC
TTCTACGAGCCCCAGATCATCACCACCGACAACACCTTCGTGTCTGGCAACTGCG
ACGTCGTGATCGGCATTGTGAACAATACCGTGTACGACCCTCTGCAGCCCGAGCT
GGACAGCTTCAAAGAGGAACTGGATAAGTACTTTAAGAACCACACAAGCCCCGA
CGTGGACCTGGGCGATATCAGCGGAATCAATGCCAGCGTCGTGAACATCCAGAA
AGAGATCGACCGGCTGAACGAGGTGGCCAAGAATCTGAACGAGAGCCTGATCGA
CCTGCAAGAACTGGGGAAGTACGAGCAGTACATCAAGTGGCCC
Amino Acid Sequence of BA.2.12.1 S371 S373 S375 (Signal peptide is in bold and
underlined; Spike ectodomain is underlined; S371 S373 S375 is in bold, underlined
and italics): SEQ ID NO: 57
MFVFLVLLPLVSSQCVNLITRTQSYTNSFTRGVYYPDKVFRSSVLHSTQDLFLPFFSN
VTWFHAIHVSGTNGTKRFDNPVLPFNDGVYFASTEKSNIIRGWIFGTTLDSKTQSLLI
VNNATNVVIKVCEFQFCNDPFLDVYYHKNNKSWMESEFRVYSSANNCTFEYVSQPF
LMDLEGKQGNFKNLREFVFKNIDGYFKIYSKHTPINLGRDLPQGFSALEPLVDLPIGIN
ITRFQTLLALHRSYLTPGDSSSGWTAGAAAYYVGYLQPRTFLLKYNENGTITDAVDC
ALDPLSETKCTLKSFTVEKGIYQTSNFRVQPTESIVRFPNITNLCPFDEVFNATRFASV
YAWNRKRISNCVADYSVLYNSASFSAFKCYGVSPTKLNDLCFTNVYADSFVIRGNE
VSQIAPGQTGNIADYNYKLPDDFTGCVIAWNSNKLDSKVGGNYNYQYRLFRKSNLK
PFERDISTEIYQAGNKPCNGVAGFNCYFPLRSYGFRPTYGVGHQPYRVVVLSFELLH
APATVCGPKKSTNLVKNKCVNFNFNGLTGTGVLTESNKKFLPFQQFGRDIADTTDA
VRDPQTLEILDITPCSFGGVSVITPGTNTSNQVAVLYQGVNCTEVPVAIHADQLTPTW
RVYSTGSNVFQTRAGCLIGAEYVNNSYECDIPIGAGICASYQTQTKSHASVASQSIIAY
TMSLGAENLVAYSNNSIAIPTNFTISVTTEILPVSMTKTSVDCTMYICGDSTECSNLLL
QYGSFCTQLKRALTGIAVEQDKNTQEVFAQVKQIYKTPPIKYFGGFNFSQILPDPSKP
SKRSPIEDLLFNKVTLADAGFIKQYGDCLGDIAARDLICAQKFNGLTVLPPLLTDEMI
AQYTSALLAGTITSGWTFGAGPALQIPFPMQMAYRENGIGVTQNVLYENQKLIANQF
NSAIGKIQDSLSSTPSALGKLQDVVNHNAQALNTLVKQLSSKFGAISSVLNDILSRLD
PPEAEVQIDRLITGRLQSLQTYVTQQLIRAAEIRASANLAATKMSECVLGQSKRVDFC
GKGYHLMSFPQSAPHGVVFLHVTYVPAQEKNFTTAPAICHDGKAHFPREGVFVSNG
THWFVTQRNFYEPQIITTDNTFVSGNCDVVIGIVNNTVYDPLQPELDSFKEELDKYFK
NHTSPDVDLGDISGINASVVNIQKEIDRLNEVAKNLNESLIDLQELGKYEQYIKWP
Nucleotide Sequence of BA.2.12.1 S371 S373 S375 (Spike ectodomain without signal
peptide is underlined; S371 S373 S375 is in bold, underlined and italics): SEQ ID
NO: 58
CAGTGTGTGAACCTGatcACAAGAACCCAGagcTACACCAACAGCTTTACCAGAGG
CGTGTACTACCCCGACAAGGTGTTCAGATCCAGCGTGCTGCACTCTACCCAGGAC
CTGTTCCTGCCTTTCTTCAGCAACGTGACCTGGTTCCACGCCATCCACGTGTCCGG
CACCAATGGCACCAAGAGATTCGACAACCCCGTGCTGCCCTTCAACGACGGGGT
GTACTTTGCCAGCACCGAGAAGTCCAACATCATCAGAGGCTGGATCTTCGGCACC
ACACTGGACAGCAAGACCCAGAGCCTGCTGATCGTGAACAACGCCACCAACGTG
GTCATCAAAGTGTGCGAGTTCCAGTTCTGCAACGACCCCTTCCTGgacGTCTACTA
TCACAAGAACAACAAGAGCTGGATGGAAAGCGAGTTCCGGGTGTACAGCAGCGC
CAACAACTGCACCTTCGAGTACGTGTCCCAGCCTTTCCTGATGGACCTGGAAGGC
AAGCAGGGCAACTTCAAGAACCTGCGCGAGTTCGTGTTCAAGAACATCGACGGC
TACTTCAAGATCTACAGCAAGCACACCCCTATCAACCTCggcCGGGATCTGCCTCA
GGGCTTCTCTGCTCTGGAACCCCTGGTGGATCTGCCCATCGGCATCAACATCACC
CGGTTTCAGACACTGCTGGCCCTGCACAGAAGCTACCTGACACCTGGCGATAGC
AGCAGCGGATGGACAGCTGGTGCCGCCGCTTACTATGTGGGCTACCTGCAGCCT
AGAACCTTTCTGCTGAAGTACAACGAGAACGGCACCATCACCGACGCCGTGGAT
TGTGCTCTGGATCCTCTGAGCGAGACAAAGTGCACCCTGAAGTCCTTCACCGTGG
AgAAGGGCATCTACCAGACCAGCAACTTCCGGGTGCAGCCCACCGAATCCATCGT
GCGGTTCCCCAATATCACCAATCTGTGCCCCTTCgacGAGGTGTTCAATGCCACCA
GATTCGCCTCTGTGTACGCCTGGAACCGGAAGCGGATCAGCAATTGCGTGGCCG
ACTACTCCGTGCTGTACAACtccGCCagcTTCagcgccTTCAAGTGCTACGGCGTGTCC
CCTACCAAGCTGAACGACCTGTGCTTCACAAACGTGTACGCCGACAGCTTCGTGA
TCCGGGGAaacGAAGTGagcCAGATTGCCCCTGGACAGACAGGCaacATCGCCGACT
ACAACTACAAGCTGCCCGACGACTTCACCGGCTGTGTGATTGCCTGGAACAGCA
ACaagCTGGACTCCAAAGTCGGCGGCAACTACAATTACcagTACCGGCTGTTCCGG
AAGTCCAATCTGAAGCCCTTCGAGCGGGACATCTCCACCGAGATCTATCAGGCC
GGCaacaagCCTTGTAACGGCGTGgccGGCTTCAACTGCTACTTCCCACTGcggTCCTA
CGGCTTTcggCCCACAtacGGCGTGGGCcacCAGCCCTACAGAGTGGTGGTGCTGAGC
TTCGAACTGCTGCATGCCCCTGCCACAGTGTGCGGCCCTAAGAAgAGCACCAATC
TCGTGAAGAACAAATGCGTGAACTTCAACTTCAACGGCCTGACCGGCACCGGCG
TGCTGACAGAGAGCAACAAGAAGTTCCTGCCATTCCAGCAGTTTGGCCGGGATA
TCGCCGATACCACAGACGCCGTTAGAGATCCCCAGACACTGGAAATCCTGGACA
TCACCCCTTGCAGCTTCGGCGGAGTGTCTGTGATCACCCCTGGCACCAACACCAG
CAATCAGGTGGCAGTGCTGTACCAGggcGTGAACTGTACCGAAGTGCCCGTGGCC
ATTCACGCCGATCAGCTGACACCTACATGGCGGGTGTACTCCACCGGCAGCAAT
GTGTTTCAGACCAGAGCCGGCTGTCTGATCGGAGCCGAGtacGTGAACAATAGCTA
CGAGTGCGACATCCCCATCGGCGCTGGCATCTGTGCCAGCTACCAGACACAGAC
AaagAGCcacGCCTCTGTGGCCAGCCAGAGCATCATTGCCTACACAATGTCTCTGGG
CGCCGAGAACctgGTGGCCTACTCCAACAACTCTATCGCTATCCCCACCAACTTCA
CCATCAGCGTGACCACAGAGATCCTGCCTGTGTCCATGACCAAGACCAGCGTGG
ACTGCACCATGTACATCTGCGGCGATTCCACCGAGTGCTCCAACCTGCTGCTGCA
GTACGGCAGCTTCTGCACCCAGCTGaagAGAGCCCTGACAGGGATCGCCGTGGAA
CAGGACAAGAACACCCAAGAGGTGTTCGCCCAAGTGAAGCAGATCTACAAGACC
CCTCCTATCAAGtacTTCGGCGGCTTCAATTTCAGCCAGATTCTGCCCGATCCTAGC
AAGCCCAGCAAGCGGAGCcctATCGAGGACCTGCTGTTCAACAAAGTGACACTGG
CCGACGCCGGCTTCATCAAGCAGTATGGCGATTGTCTGGGCGACATTGCCGCCAG
GGATCTGATTTGCGCCCAGAAGTTTAACGGACTGACAGTGCTGCCTCCTCTGCTG
ACCGATGAGATGATCGCCCAGTACACATCTGCCCTGCTGGCCGGCACAATCACA
AGCGGCTGGACATTTGGAGCTGGCcctGCTCTGCAGATCCCCTTTccaATGCAGATG
GCCTACCGGTTCAACGGCATCGGAGTGACCCAGAATGTGCTGTACGAGAACCAG
AAGCTGATCGCCAACCAGTTCAACAGCGCCATCGGCAAGATCCAGGACAGCCTG
AGCAGCACAcccAGCGCCCTGGGAAAGCTGCAGGACGTGGTCAACcacAATGCCCA
GGCACTGAACACCCTGGTCAAGCAGCTGTCCTCCaagTTCGGCGCCATCAGCTCTG
TGCTGAACGATATCCTGAGCAGACTGGACccccctGAAGCCGAGGTGCAGATCGAC
AGACTGATCACCGGAAGGCTGCAGTCCCTGCAGACCTACGTTACCCAGCAGCTG
ATCAGAGCCGCCGAGATTAGAGCCTCTGCCAATCTGGCCGCCACCAAGATGTCT
GAGTGTGTGCTGGGCCAGAGCAAGAGAGTGGACTTTTGCGGCAAGGGCTACCAC
CTGATGAGCTTCCCTCAGTCTGCCCCTCACGGCGTGGTGTTTCTGCACGTGACAT
ACGTGCCCGCTCAAGAGAAGAATTTCACCACCGCTCCAGCCATCTGCCACGACG
GCAAAGCCCACTTTCCTAGAGAAGGCGTGTTCGTGTCCAACGGCACCCATTGGTT
CGTGACCCAGCGGAACTTCTACGAGCCCCAGATCATCACCACCGACAACACCTTC
GTGTCTGGCAACTGCGACGTCGTGATCGGCATTGTGAACAATACCGTGTACGACC
CTCTGCAGCCCGAGCTGGACAGCTTCAAAGAGGAACTGGATAAGTACTTTAAGA
ACCACACAAGCCCCGACGTGGACCTGGGCGATATCAGCGGAATCAATGCCAGCG
TCGTGAACATCCAGAAAGAGATCGACCGGCTGAACGAGGTGGCCAAGAATCTGA
ACGAGAGCCTGATCGACCTGCAAGAACTGGGGAAGTACGAGCAGTACATCAAGT
GGCCC
Amino Acid Sequence of BA.2.12.1 S371 S373 S375 (Spike ectodomain without signal
peptide is underlined; S371 S373 S375 is in bold, underlined and italics): SEQ ID
NO: 59
QCVNLITRTQSYTNSFTRGVYYPDKVFRSSVLHSTQDLFLPFFSNVTWFHAIHVSGTN
GTKRFDNPVLPFNDGVYFASTEKSNIIRGWIFGTTLDSKTQSLLIVNNATNVVIKVCEF
QFCNDPFLDVYYHKNNKSWMESEFRVYSSANNCTFEYVSQPFLMDLEGKQGNFKN
LREFVFKNIDGYFKIYSKHTPINLGRDLPQGFSALEPLVDLPIGINITRFQTLLALHRSY
LTPGDSSSGWTAGAAAYYVGYLQPRTFLLKYNENGTITDAVDCALDPLSETKCTLKS
FTVEKGIYQTSNFRVQPTESIVRFPNITNLCPFDEVFNATRFASVYAWNRKRISNCVA
DYSVLYNSASFSAFKCYGVSPTKLNDLCFTNVYADSFVIRGNEVSQIAPGQTGNIAD
YNYKLPDDFTGCVIAWNSNKLDSKVGGNYNYQYRLFRKSNLKPFERDISTEIYQAG
NKPCNGVAGFNCYFPLRSYGFRPTYGVGHQPYRVVVLSFELLHAPATVCGPKKSTN
LVKNKCVNFNFNGLTGTGVLTESNKKFLPFQQFGRDIADTTDAVRDPQTLEILDITPC
SFGGVSVITPGTNTSNQVAVLYQGVNCTEVPVAIHADQLTPTWRVYSTGSNVFQTRA
GCLIGAEYVNNSYECDIPIGAGICASYQTQTKSHASVASQSIIAYTMSLGAENLVAYS
NNSIAIPTNFTISVTTEILPVSMTKTSVDCTMYICGDSTECSNLLLQYGSFCTQLKRAL
TGIAVEQDKNTQEVFAQVKQIYKTPPIKYFGGFNFSQILPDPSKPSKRSPIEDLLFNKV
TLADAGFIKQYGDCLGDIAARDLICAQKFNGLTVLPPLLTDEMIAQYTSALLAGTITS
GWTFGAGPALQIPFPMQMAYRENGIGVTQNVLYENQKLIANQFNSAIGKIQDSLSSTP
SALGKLQDVVNHNAQALNTLVKQLSSKFGAISSVLNDILSRLDPPEAEVQIDRLITGR
LQSLQTYVTQQLIRAAEIRASANLAATKMSECVLGQSKRVDFCGKGYHLMSFPQSAP
HGVVFLHVTYVPAQEKNFTTAPAICHDGKAHFPREGVFVSNGTHWFVTQRNFYEPQ
IITTDNTFVSGNCDVVIGIVNNTVYDPLQPELDSFKEELDKYFKNHTSPDVDLGDISGI
NASVVNIQKEIDRLNEVAKNLNESLIDLQELGKYEQYIKWP
Nucleotide Sequence of BA.4/5 (Signal peptide is in bold and underlined; GS linker
is in italics; TM and CT From F is in bold and double underlined; Spike ectodomain
is underlined): SEQ ID NO: 60
ATGTTCGTGTTTCTGGTGCTGCTGCCTCTGGTGTCCAGCCAGTGTGTGAACCT
GatcACAAGAACCCAGagcTACACCAACAGCTTTACCAGAGGCGTGTACTACCCCG
ACAAGGTGTTCAGATCCAGCGTGCTGCACTCTACCCAGGACCTGTTCCTGCCTTT
CTTCAGCAACGTGACCTGGTTCCACGCCATCTCCGGCACCAATGGCACCAAGAG
ATTCGACAACCCCGTGCTGCCCTTCAACGACGGGGTGTACTTTGCCAGCACCGAG
AAGTCCAACATCATCAGAGGCTGGATCTTCGGCACCACACTGGACAGCAAGACC
CAGAGCCTGCTGATCGTGAACAACGCCACCAACGTGGTCATCAAAGTGTGCGAG
TTCCAGTTCTGCAACGACCCCTTCCTGgacGTCTACTATCACAAGAACAACAAGAG
CTGGATGGAAAGCGAGTTCCGGGTGTACAGCAGCGCCAACAACTGCACCTTCGA
GTACGTGTCCCAGCCTTTCCTGATGGACCTGGAAGGCAAGCAGGGCAACTTCAA
GAACCTGCGCGAGTTCGTGTTCAAGAACATCGACGGCTACTTCAAGATCTACAGC
AAGCACACCCCTATCAACCTCggcCGGGATCTGCCTCAGGGCTTCTCTGCTCTGGA
ACCCCTGGTGGATCTGCCCATCGGCATCAACATCACCCGGTTTCAGACACTGCTG
GCCCTGCACAGAAGCTACCTGACACCTGGCGATAGCAGCAGCGGATGGACAGCT
GGTGCCGCCGCTTACTATGTGGGCTACCTGCAGCCTAGAACCTTTCTGCTGAAGT
ACAACGAGAACGGCACCATCACCGACGCCGTGGATTGTGCTCTGGATCCTCTGA
GCGAGACAAAGTGCACCCTGAAGTCCTTCACCGTGGAgAAGGGCATCTACCAGA
CCAGCAACTTCCGGGTGCAGCCCACCGAATCCATCGTGCGGTTCCCCAATATCAC
CAATCTGTGCCCCTTCgacGAGGTGTTCAATGCCACCAGATTCGCCTCTGTGTACG
CCTGGAACCGGAAGCGGATCAGCAATTGCGTGGCCGACTACTCCGTGCTGTACA
ACttcGCCcctTTCttcgccTTCAAGTGCTACGGCGTGTCCCCTACCAAGCTGAACGACC
TGTGCTTCACAAACGTGTACGCCGACAGCTTCGTGATCCGGGGAaacGAAGTGagcC
AGATTGCCCCTGGACAGACAGGCaacATCGCCGACTACAACTACAAGCTGCCCGA
CGACTTCACCGGCTGTGTGATTGCCTGGAACAGCAACaagCTGGACTCCAAAGTCg
gcGGCAACTACAATTACcggTACCGGCTGTTCCGGAAGTCCAATCTGAAGCCCTTC
GAGCGGGACATCTCCACCGAGATCTATCAGGCCGGCaacaagCCTTGTAACGGCGT
GgccGGCgtgAACTGCTACTTCCCACTGcagTCCTACGGCTTTcggCCCACAtacGGCGT
GGGCcacCAGCCCTACAGAGTGGTGGTGCTGAGCTTCGAACTGCTGCATGCCCCTG
CCACAGTGTGCGGCCCTAAGAAgAGCACCAATCTCGTGAAGAACAAATGCGTGA
ACTTCAACTTCAACGGCCTGACCGGCACCGGCGTGCTGACAGAGAGCAACAAGA
AGTTCCTGCCATTCCAGCAGTTTGGCCGGGATATCGCCGATACCACAGACGCCGT
TAGAGATCCCCAGACACTGGAAATCCTGGACATCACCCCTTGCAGCTTCGGCGG
AGTGTCTGTGATCACCCCTGGCACCAACACCAGCAATCAGGTGGCAGTGCTGTAC
CAGggcGTGAACTGTACCGAAGTGCCCGTGGCCATTCACGCCGATCAGCTGACAC
CTACATGGCGGGTGTACTCCACCGGCAGCAATGTGTTTCAGACCAGAGCCGGCT
GTCTGATCGGAGCCGAGtacGTGAACAATAGCTACGAGTGCGACATCCCCATCGG
CGCTGGCATCTGTGCCAGCTACCAGACACAGACAaagAGCcacGCCTCTGTGGCCA
GCCAGAGCATCATTGCCTACACAATGTCTCTGGGCGCCGAGAACAGCGTGGCCT
ACTCCAACAACTCTATCGCTATCCCCACCAACTTCACCATCAGCGTGACCACAGA
GATCCTGCCTGTGTCCATGACCAAGACCAGCGTGGACTGCACCATGTACATCTGC
GGCGATTCCACCGAGTGCTCCAACCTGCTGCTGCAGTACGGCAGCTTCTGCACCC
AGCTGaagAGAGCCCTGACAGGGATCGCCGTGGAACAGGACAAGAACACCCAAG
AGGTGTTCGCCCAAGTGAAGCAGATCTACAAGACCCCTCCTATCAAGtacTTCGGC
GGCTTCAATTTCAGCCAGATTCTGCCCGATCCTAGCAAGCCCAGCAAGCGGAGCc
ctATCGAGGACCTGCTGTTCAACAAAGTGACACTGGCCGACGCCGGCTTCATCAA
GCAGTATGGCGATTGTCTGGGCGACATTGCCGCCAGGGATCTGATTTGCGCCCAG
AAGTTTAACGGACTGACAGTGCTGCCTCCTCTGCTGACCGATGAGATGATCGCCC
AGTACACATCTGCCCTGCTGGCCGGCACAATCACAAGCGGCTGGACATTTGGAG
CTGGCcctGCTCTGCAGATCCCCTTTccaATGCAGATGGCCTACCGGTTCAACGGCA
TCGGAGTGACCCAGAATGTGCTGTACGAGAACCAGAAGCTGATCGCCAACCAGT
TCAACAGCGCCATCGGCAAGATCCAGGACAGCCTGAGCAGCACAcccAGCGCCCT
GGGAAAGCTGCAGGACGTGGTCAACcacAATGCCCAGGCACTGAACACCCTGGTC
AAGCAGCTGTCCTCCaagTTCGGCGCCATCAGCTCTGTGCTGAACGATATCCTGAG
CAGACTGGACccccctGAAGCCGAGGTGCAGATCGACAGACTGATCACCGGAAGGC
TGCAGTCCCTGCAGACCTACGTTACCCAGCAGCTGATCAGAGCCGCCGAGATTA
GAGCCTCTGCCAATCTGGCCGCCACCAAGATGTCTGAGTGTGTGCTGGGCCAGA
GCAAGAGAGTGGACTTTTGCGGCAAGGGCTACCACCTGATGAGCTTCCCTCAGTC
TGCCCCTCACGGCGTGGTGTTTCTGCACGTGACATACGTGCCCGCTCAAGAGAAG
AATTTCACCACCGCTCCAGCCATCTGCCACGACGGCAAAGCCCACTTTCCTAGAG
AAGGCGTGTTCGTGTCCAACGGCACCCATTGGTTCGTGACCCAGCGGAACTTCTA
CGAGCCCCAGATCATCACCACCGACAACACCTTCGTGTCTGGCAACTGCGACGTC
GTGATCGGCATTGTGAACAATACCGTGTACGACCCTCTGCAGCCCGAGCTGGAC
AGCTTCAAAGAGGAACTGGATAAGTACTTTAAGAACCACACAAGCCCCGACGTG
GACCTGGGCGATATCAGCGGAATCAATGCCAGCGTCGTGAACATCCAGAAAGAG
ATCGACCGGCTGAACGAGGTGGCCAAGAATCTGAACGAGAGCCTGATCGACCTG
CAAGAACTGGGGAAGTACGAGCAGTACATCAAGTGGCCCggcggaggtgggtcgCTCAT
AACATACATCGTCCTGACTATAATCAGCTTGGTATTTGGTATTTTGTCTTTGA
TTCTTGCATGCTATTTGATGTATAAACAGAAAGCTCAGCAGAAGACTCTCCT
GTGGCTCGGTAACAACACACTCGACCAGATGAGAGCAACTACAAAGATGTG
A
Amino Acid Sequence of BA.4/5 (Signal peptide is in bold and underlined; GS linker
is in italics; TM and CT From F is in bold and double underlined; Spike ectodomain
is underlined): SEQ ID NO: 61
MFVFLVLLPLVSSQCVNLITRTQSYTNSFTRGVYYPDKVFRSSVLHSTQDLFLPFFSN
VTWFHAISGTNGTKRFDNPVLPFNDGVYFASTEKSNIIRGWIFGTTLDSKTQSLLIVN
NATNVVIKVCEFQFCNDPFLDVYYHKNNKSWMESEFRVYSSANNCTFEYVSQPFLM
DLEGKQGNFKNLREFVFKNIDGYFKIYSKHTPINLGRDLPQGFSALEPLVDLPIGINIT
RFQTLLALHRSYLTPGDSSSGWTAGAAAYYVGYLQPRTFLLKYNENGTITDAVDCA
LDPLSETKCTLKSFTVEKGIYQTSNFRVQPTESIVRFPNITNLCPFDEVFNATRFASVY
AWNRKRISNCVADYSVLYNFAPFFAFKCYGVSPTKLNDLCFTNVYADSFVIRGNEVS
QIAPGQTGNIADYNYKLPDDFTGCVIAWNSNKLDSKVGGNYNYRYRLFRKSNLKPF
ERDISTEIYQAGNKPCNGVAGVNCYFPLQSYGFRPTYGVGHQPYRVVVLSFELLHAP
ATVCGPKKSTNLVKNKCVNFNFNGLTGTGVLTESNKKFLPFQQFGRDIADTTDAVR
DPQTLEILDITPCSFGGVSVITPGTNTSNQVAVLYQGVNCTEVPVAIHADQLTPTWRV
YSTGSNVFQTRAGCLIGAEYVNNSYECDIPIGAGICASYQTQTKSHASVASQSIIAYT
MSLGAENSVAYSNNSIAIPTNFTISVTTEILPVSMTKTSVDCTMYICGDSTECSNLLLQ
YGSFCTQLKRALTGIAVEQDKNTQEVFAQVKQIYKTPPIKYFGGFNFSQILPDPSKPS
KRSPIEDLLFNKVTLADAGFIKQYGDCLGDIAARDLICAQKFNGLTVLPPLLTDEMIA
QYTSALLAGTITSGWTFGAGPALQIPFPMQMAYRENGIGVTQNVLYENQKLIANQFN
SAIGKIQDSLSSTPSALGKLQDVVNHNAQALNTLVKQLSSKFGAISSVLNDILSRLDPP
EAEVQIDRLITGRLQSLQTYVTQQLIRAAEIRASANLAATKMSECVLGQSKRVDFCG
KGYHLMSFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKEELDKYFKN
HTSPDVDLGDISGINASVVNIQKEIDRLNEVAKNLNESLIDLQELGKYEQYIKWPGGG
GSLITYIVLTIISLVFGILSLILACYLMYKQKAQQKTLLWLGNNTLDQMRATTK
M*
Nucleotide Sequence of BA.4/5 (Signal peptide is in bold and underlined; Spike
ectodomain is underlined): SEQ ID NO: 62
ATGTTCGTGTTTCTGGTGCTGCTGCCTCTGGTGTCCAGCCAGTGTGTGAACCT
GatcACAAGAACCCAGagcTACACCAACAGCTTTACCAGAGGCGTGTACTACCCCG
ACAAGGTGTTCAGATCCAGCGTGCTGCACTCTACCCAGGACCTGTTCCTGCCTTT
CTTCAGCAACGTGACCTGGTTCCACGCCATCTCCGGCACCAATGGCACCAAGAG
ATTCGACAACCCCGTGCTGCCCTTCAACGACGGGGTGTACTTTGCCAGCACCGAG
AAGTCCAACATCATCAGAGGCTGGATCTTCGGCACCACACTGGACAGCAAGACC
CAGAGCCTGCTGATCGTGAACAACGCCACCAACGTGGTCATCAAAGTGTGCGAG
TTCCAGTTCTGCAACGACCCCTTCCTGgacGTCTACTATCACAAGAACAACAAGAG
CTGGATGGAAAGCGAGTTCCGGGTGTACAGCAGCGCCAACAACTGCACCTTCGA
GTACGTGTCCCAGCCTTTCCTGATGGACCTGGAAGGCAAGCAGGGCAACTTCAA
GAACCTGCGCGAGTTCGTGTTCAAGAACATCGACGGCTACTTCAAGATCTACAGC
AAGCACACCCCTATCAACCTCggcCGGGATCTGCCTCAGGGCTTCTCTGCTCTGGA
ACCCCTGGTGGATCTGCCCATCGGCATCAACATCACCCGGTTTCAGACACTGCTG
GCCCTGCACAGAAGCTACCTGACACCTGGCGATAGCAGCAGCGGATGGACAGCT
GGTGCCGCCGCTTACTATGTGGGCTACCTGCAGCCTAGAACCTTTCTGCTGAAGT
ACAACGAGAACGGCACCATCACCGACGCCGTGGATTGTGCTCTGGATCCTCTGA
GCGAGACAAAGTGCACCCTGAAGTCCTTCACCGTGGAgAAGGGCATCTACCAGA
CCAGCAACTTCCGGGTGCAGCCCACCGAATCCATCGTGCGGTTCCCCAATATCAC
CAATCTGTGCCCCTTCgacGAGGTGTTCAATGCCACCAGATTCGCCTCTGTGTACG
CCTGGAACCGGAAGCGGATCAGCAATTGCGTGGCCGACTACTCCGTGCTGTACA
ACttcGCCcctTTCttcgccTTCAAGTGCTACGGCGTGTCCCCTACCAAGCTGAACGACC
TGTGCTTCACAAACGTGTACGCCGACAGCTTCGTGATCCGGGGAaacGAAGTGagcC
AGATTGCCCCTGGACAGACAGGCaacATCGCCGACTACAACTACAAGCTGCCCGA
CGACTTCACCGGCTGTGTGATTGCCTGGAACAGCAACaagCTGGACTCCAAAGTCg
gcGGCAACTACAATTACcggTACCGGCTGTTCCGGAAGTCCAATCTGAAGCCCTTC
GAGCGGGACATCTCCACCGAGATCTATCAGGCCGGCaacaagCCTTGTAACGGCGT
GgccGGCgtgAACTGCTACTTCCCACTGcagTCCTACGGCTTTcggCCCACAtacGGCGT
GGGCcacCAGCCCTACAGAGTGGTGGTGCTGAGCTTCGAACTGCTGCATGCCCCTG
CCACAGTGTGCGGCCCTAAGAAgAGCACCAATCTCGTGAAGAACAAATGCGTGA
ACTTCAACTTCAACGGCCTGACCGGCACCGGCGTGCTGACAGAGAGCAACAAGA
AGTTCCTGCCATTCCAGCAGTTTGGCCGGGATATCGCCGATACCACAGACGCCGT
TAGAGATCCCCAGACACTGGAAATCCTGGACATCACCCCTTGCAGCTTCGGCGG
AGTGTCTGTGATCACCCCTGGCACCAACACCAGCAATCAGGTGGCAGTGCTGTAC
CAGggcGTGAACTGTACCGAAGTGCCCGTGGCCATTCACGCCGATCAGCTGACAC
CTACATGGCGGGTGTACTCCACCGGCAGCAATGTGTTTCAGACCAGAGCCGGCT
GTCTGATCGGAGCCGAGtacGTGAACAATAGCTACGAGTGCGACATCCCCATCGG
CGCTGGCATCTGTGCCAGCTACCAGACACAGACAaagAGCcacGCCTCTGTGGCCA
GCCAGAGCATCATTGCCTACACAATGTCTCTGGGCGCCGAGAACAGCGTGGCCT
ACTCCAACAACTCTATCGCTATCCCCACCAACTTCACCATCAGCGTGACCACAGA
GATCCTGCCTGTGTCCATGACCAAGACCAGCGTGGACTGCACCATGTACATCTGC
GGCGATTCCACCGAGTGCTCCAACCTGCTGCTGCAGTACGGCAGCTTCTGCACCC
AGCTGaagAGAGCCCTGACAGGGATCGCCGTGGAACAGGACAAGAACACCCAAG
AGGTGTTCGCCCAAGTGAAGCAGATCTACAAGACCCCTCCTATCAAGtacTTCGGC
GGCTTCAATTTCAGCCAGATTCTGCCCGATCCTAGCAAGCCCAGCAAGCGGAGCc
ctATCGAGGACCTGCTGTTCAACAAAGTGACACTGGCCGACGCCGGCTTCATCAA
GCAGTATGGCGATTGTCTGGGCGACATTGCCGCCAGGGATCTGATTTGCGCCCAG
AAGTTTAACGGACTGACAGTGCTGCCTCCTCTGCTGACCGATGAGATGATCGCCC
AGTACACATCTGCCCTGCTGGCCGGCACAATCACAAGCGGCTGGACATTTGGAG
CTGGCcctGCTCTGCAGATCCCCTTTccaATGCAGATGGCCTACCGGTTCAACGGCA
TCGGAGTGACCCAGAATGTGCTGTACGAGAACCAGAAGCTGATCGCCAACCAGT
TCAACAGCGCCATCGGCAAGATCCAGGACAGCCTGAGCAGCACAcccAGCGCCCT
GGGAAAGCTGCAGGACGTGGTCAACcacAATGCCCAGGCACTGAACACCCTGGTC
AAGCAGCTGTCCTCCaagTTCGGCGCCATCAGCTCTGTGCTGAACGATATCCTGAG
CAGACTGGACccccctGAAGCCGAGGTGCAGATCGACAGACTGATCACCGGAAGGC
TGCAGTCCCTGCAGACCTACGTTACCCAGCAGCTGATCAGAGCCGCCGAGATTA
GAGCCTCTGCCAATCTGGCCGCCACCAAGATGTCTGAGTGTGTGCTGGGCCAGA
GCAAGAGAGTGGACTTTTGCGGCAAGGGCTACCACCTGATGAGCTTCCCTCAGTC
TGCCCCTCACGGCGTGGTGTTTCTGCACGTGACATACGTGCCCGCTCAAGAGAAG
AATTTCACCACCGCTCCAGCCATCTGCCACGACGGCAAAGCCCACTTTCCTAGAG
AAGGCGTGTTCGTGTCCAACGGCACCCATTGGTTCGTGACCCAGCGGAACTTCTA
CGAGCCCCAGATCATCACCACCGACAACACCTTCGTGTCTGGCAACTGCGACGTC
GTGATCGGCATTGTGAACAATACCGTGTACGACCCTCTGCAGCCCGAGCTGGAC
AGCTTCAAAGAGGAACTGGATAAGTACTTTAAGAACCACACAAGCCCCGACGTG
GACCTGGGCGATATCAGCGGAATCAATGCCAGCGTCGTGAACATCCAGAAAGAG
ATCGACCGGCTGAACGAGGTGGCCAAGAATCTGAACGAGAGCCTGATCGACCTG
CAAGAACTGGGGAAGTACGAGCAGTACATCAAGTGGCCC
Amino Acid Sequence of BA.4/5 (Signal peptide is in bold and underlined; Spike
ectodomain is underlined): SEQ ID NO: 63
MFVFLVLLPLVSSQCVNLITRTQSYTNSFTRGVYYPDKVFRSSVLHSTQDLFLPFFSN
VTWFHAISGTNGTKRFDNPVLPFNDGVYFASTEKSNIIRGWIFGTTLDSKTQSLLIVN
NATNVVIKVCEFQFCNDPFLDVYYHKNNKSWMESEFRVYSSANNCTFEYVSQPFLM
DLEGKQGNFKNLREFVFKNIDGYFKIYSKHTPINLGRDLPQGFSALEPLVDLPIGINIT
RFQTLLALHRSYLTPGDSSSGWTAGAAAYYVGYLQPRTFLLKYNENGTITDAVDCA
LDPLSETKCTLKSFTVEKGIYQTSNFRVQPTESIVRFPNITNLCPFDEVFNATRFASVY
AWNRKRISNCVADYSVLYNFAPFFAFKCYGVSPTKLNDLCFTNVYADSFVIRGNEVS
QIAPGQTGNIADYNYKLPDDFTGCVIAWNSNKLDSKVGGNYNYRYRLFRKSNLKPF
ERDISTEIYQAGNKPCNGVAGVNCYFPLQSYGFRPTYGVGHQPYRVVVLSFELLHAP
ATVCGPKKSTNLVKNKCVNFNFNGLTGTGVLTESNKKFLPFQQFGRDIADTTDAVR
DPQTLEILDITPCSFGGVSVITPGTNTSNQVAVLYQGVNCTEVPVAIHADQLTPTWRV
YSTGSNVFQTRAGCLIGAEYVNNSYECDIPIGAGICASYQTQTKSHASVASQSIIAYT
MSLGAENSVAYSNNSIAIPTNFTISVTTEILPVSMTKTSVDCTMYICGDSTECSNLLLQ
YGSFCTQLKRALTGIAVEQDKNTQEVFAQVKQIYKTPPIKYFGGFNFSQILPDPSKPS
KRSPIEDLLFNKVTLADAGFIKQYGDCLGDIAARDLICAQKFNGLTVLPPLLTDEMIA
QYTSALLAGTITSGWTFGAGPALQIPFPMQMAYRENGIGVTQNVLYENQKLIANQFN
SAIGKIQDSLSSTPSALGKLQDVVNHNAQALNTLVKQLSSKFGAISSVLNDILSRLDPP
EAEVQIDRLITGRLQSLQTYVTQQLIRAAEIRASANLAATKMSECVLGQSKRVDFCG
KGYHLMSFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKEELDKYFKN
HTSPDVDLGDISGINASVVNIQKEIDRLNEVAKNLNESLIDLQELGKYEQYIKWP
Nucleotide Sequence of BA.4/5 (Spike ectodomain without signal peptide): SEQ ID
NO: 64
CAGTGTGTGAACCTGatcACAAGAACCCAGagcTACACCAACAGCTTTACCAGAGG
CGTGTACTACCCCGACAAGGTGTTCAGATCCAGCGTGCTGCACTCTACCCAGGAC
CTGTTCCTGCCTTTCTTCAGCAACGTGACCTGGTTCCACGCCATCTCCGGCACCAA
TGGCACCAAGAGATTCGACAACCCCGTGCTGCCCTTCAACGACGGGGTGTACTTT
GCCAGCACCGAGAAGTCCAACATCATCAGAGGCTGGATCTTCGGCACCACACTG
GACAGCAAGACCCAGAGCCTGCTGATCGTGAACAACGCCACCAACGTGGTCATC
AAAGTGTGCGAGTTCCAGTTCTGCAACGACCCCTTCCTGgacGTCTACTATCACAA
GAACAACAAGAGCTGGATGGAAAGCGAGTTCCGGGTGTACAGCAGCGCCAACA
ACTGCACCTTCGAGTACGTGTCCCAGCCTTTCCTGATGGACCTGGAAGGCAAGCA
GGGCAACTTCAAGAACCTGCGCGAGTTCGTGTTCAAGAACATCGACGGCTACTTC
AAGATCTACAGCAAGCACACCCCTATCAACCTCggcCGGGATCTGCCTCAGGGCTT
CTCTGCTCTGGAACCCCTGGTGGATCTGCCCATCGGCATCAACATCACCCGGTTT
CAGACACTGCTGGCCCTGCACAGAAGCTACCTGACACCTGGCGATAGCAGCAGC
GGATGGACAGCTGGTGCCGCCGCTTACTATGTGGGCTACCTGCAGCCTAGAACCT
TTCTGCTGAAGTACAACGAGAACGGCACCATCACCGACGCCGTGGATTGTGCTCT
GGATCCTCTGAGCGAGACAAAGTGCACCCTGAAGTCCTTCACCGTGGAgAAGGG
CATCTACCAGACCAGCAACTTCCGGGTGCAGCCCACCGAATCCATCGTGCGGTTC
CCCAATATCACCAATCTGTGCCCCTTCgacGAGGTGTTCAATGCCACCAGATTCGC
CTCTGTGTACGCCTGGAACCGGAAGCGGATCAGCAATTGCGTGGCCGACTACTCC
GTGCTGTACAACttcGCCcctTTCttcgccTTCAAGTGCTACGGCGTGTCCCCTACCAAG
CTGAACGACCTGTGCTTCACAAACGTGTACGCCGACAGCTTCGTGATCCGGGGAa
acGAAGTGagcCAGATTGCCCCTGGACAGACAGGCaacATCGCCGACTACAACTACA
AGCTGCCCGACGACTTCACCGGCTGTGTGATTGCCTGGAACAGCAACaagCTGGA
CTCCAAAGTCggcGGCAACTACAATTACcggTACCGGCTGTTCCGGAAGTCCAATCT
GAAGCCCTTCGAGCGGGACATCTCCACCGAGATCTATCAGGCCGGCaacaagCCTTG
TAACGGCGTGgccGGCgtgAACTGCTACTTCCCACTGcagTCCTACGGCTTTcggCCCA
CAtacGGCGTGGGCcacCAGCCCTACAGAGTGGTGGTGCTGAGCTTCGAACTGCTGC
ATGCCCCTGCCACAGTGTGCGGCCCTAAGAAgAGCACCAATCTCGTGAAGAACA
AATGCGTGAACTTCAACTTCAACGGCCTGACCGGCACCGGCGTGCTGACAGAGA
GCAACAAGAAGTTCCTGCCATTCCAGCAGTTTGGCCGGGATATCGCCGATACCAC
AGACGCCGTTAGAGATCCCCAGACACTGGAAATCCTGGACATCACCCCTTGCAG
CTTCGGCGGAGTGTCTGTGATCACCCCTGGCACCAACACCAGCAATCAGGTGGC
AGTGCTGTACCAGggcGTGAACTGTACCGAAGTGCCCGTGGCCATTCACGCCGAT
CAGCTGACACCTACATGGCGGGTGTACTCCACCGGCAGCAATGTGTTTCAGACCA
GAGCCGGCTGTCTGATCGGAGCCGAGtacGTGAACAATAGCTACGAGTGCGACAT
CCCCATCGGCGCTGGCATCTGTGCCAGCTACCAGACACAGACAaagAGCcacGCCT
CTGTGGCCAGCCAGAGCATCATTGCCTACACAATGTCTCTGGGCGCCGAGAACA
GCGTGGCCTACTCCAACAACTCTATCGCTATCCCCACCAACTTCACCATCAGCGT
GACCACAGAGATCCTGCCTGTGTCCATGACCAAGACCAGCGTGGACTGCACCAT
GTACATCTGCGGCGATTCCACCGAGTGCTCCAACCTGCTGCTGCAGTACGGCAGC
TTCTGCACCCAGCTGaagAGAGCCCTGACAGGGATCGCCGTGGAACAGGACAAGA
ACACCCAAGAGGTGTTCGCCCAAGTGAAGCAGATCTACAAGACCCCTCCTATCA
AGtacTTCGGCGGCTTCAATTTCAGCCAGATTCTGCCCGATCCTAGCAAGCCCAGC
AAGCGGAGCcctATCGAGGACCTGCTGTTCAACAAAGTGACACTGGCCGACGCCG
GCTTCATCAAGCAGTATGGCGATTGTCTGGGCGACATTGCCGCCAGGGATCTGAT
TTGCGCCCAGAAGTTTAACGGACTGACAGTGCTGCCTCCTCTGCTGACCGATGAG
ATGATCGCCCAGTACACATCTGCCCTGCTGGCCGGCACAATCACAAGCGGCTGG
ACATTTGGAGCTGGCcctGCTCTGCAGATCCCCTTTccaATGCAGATGGCCTACCGG
TTCAACGGCATCGGAGTGACCCAGAATGTGCTGTACGAGAACCAGAAGCTGATC
GCCAACCAGTTCAACAGCGCCATCGGCAAGATCCAGGACAGCCTGAGCAGCACA
cccAGCGCCCTGGGAAAGCTGCAGGACGTGGTCAACcacAATGCCCAGGCACTGAA
CACCCTGGTCAAGCAGCTGTCCTCCaagTTCGGCGCCATCAGCTCTGTGCTGAACG
ATATCCTGAGCAGACTGGACccccctGAAGCCGAGGTGCAGATCGACAGACTGATC
ACCGGAAGGCTGCAGTCCCTGCAGACCTACGTTACCCAGCAGCTGATCAGAGCC
GCCGAGATTAGAGCCTCTGCCAATCTGGCCGCCACCAAGATGTCTGAGTGTGTGC
TGGGCCAGAGCAAGAGAGTGGACTTTTGCGGCAAGGGCTACCACCTGATGAGCT
TCCCTCAGTCTGCCCCTCACGGCGTGGTGTTTCTGCACGTGACATACGTGCCCGC
TCAAGAGAAGAATTTCACCACCGCTCCAGCCATCTGCCACGACGGCAAAGCCCA
CTTTCCTAGAGAAGGCGTGTTCGTGTCCAACGGCACCCATTGGTTCGTGACCCAG
CGGAACTTCTACGAGCCCCAGATCATCACCACCGACAACACCTTCGTGTCTGGCA
ACTGCGACGTCGTGATCGGCATTGTGAACAATACCGTGTACGACCCTCTGCAGCC
CGAGCTGGACAGCTTCAAAGAGGAACTGGATAAGTACTTTAAGAACCACACAAG
CCCCGACGTGGACCTGGGCGATATCAGCGGAATCAATGCCAGCGTCGTGAACAT
CCAGAAAGAGATCGACCGGCTGAACGAGGTGGCCAAGAATCTGAACGAGAGCC
TGATCGACCTGCAAGAACTGGGGAAGTACGAGCAGTACATCAAGTGGCCC
Amino Acid Sequence of BA.4/5 (Spike ectodomain without signal peptide): SEQ ID
NO: 65
QCVNLITRTQSYTNSFTRGVYYPDKVFRSSVLHSTQDLFLPFFSNVTWFHAISGTNGT
KRFDNPVLPFNDGVYFASTEKSNIIRGWIFGTTLDSKTQSLLIVNNATNVVIKVCEFQF
CNDPFLDVYYHKNNKSWMESEFRVYSSANNCTFEYVSQPFLMDLEGKQGNFKNLR
EFVFKNIDGYFKIYSKHTPINLGRDLPQGFSALEPLVDLPIGINITRFQTLLALHRSYLT
PGDSSSGWTAGAAAYYVGYLQPRTFLLKYNENGTITDAVDCALDPLSETKCTLKSFT
VEKGIYQTSNFRVQPTESIVRFPNITNLCPFDEVFNATRFASVYAWNRKRISNCVADY
SVLYNFAPFFAFKCYGVSPTKLNDLCFTNVYADSFVIRGNEVSQIAPGQTGNIADYN
YKLPDDFTGCVIAWNSNKLDSKVGGNYNYRYRLFRKSNLKPFERDISTEIYQAGNKP
CNGVAGVNCYFPLQSYGFRPTYGVGHQPYRVVVLSFELLHAPATVCGPKKSTNLVK
NKCVNFNFNGLTGTGVLTESNKKFLPFQQFGRDIADTTDAVRDPQTLEILDITPCSFG
GVSVITPGTNTSNQVAVLYQGVNCTEVPVAIHADQLTPTWRVYSTGSNVFQTRAGC
LIGAEYVNNSYECDIPIGAGICASYQTQTKSHASVASQSIIAYTMSLGAENSVAYSNN
SIAIPTNFTISVTTEILPVSMTKTSVDCTMYICGDSTECSNLLLQYGSFCTQLKRALTGI
AVEQDKNTQEVFAQVKQIYKTPPIKYFGGFNFSQILPDPSKPSKRSPIEDLLFNKVTLA
DAGFIKQYGDCLGDIAARDLICAQKFNGLTVLPPLLTDEMIAQYTSALLAGTITSGWT
FGAGPALQIPFPMQMAYRENGIGVTQNVLYENQKLIANQFNSAIGKIQDSLSSTPSAL
GKLQDVVNHNAQALNTLVKQLSSKFGAISSVLNDILSRLDPPEAEVQIDRLITGRLQS
LQTYVTQQLIRAAEIRASANLAATKMSECVLGQSKRVDFCGKGYHLMSFPQSAPHG
VVFLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISGINA
SVVNIQKEIDRLNEVAKNLNESLIDLQELGKYEQYIKWP
Nucleotide Sequence of BA.4/5 S371 S373 S375 (Signal peptide is in bold and
underlined; GS linker is in italics; TM and CT From F is in bold and double
underlined; Spike ectodomain is underlined; S371 S373 S375 is in bold, underlined
and italics): SEQ ID NO: 66
ATGTTCGTGTTTCTGGTGCTGCTGCCTCTGGTGTCCAGCCAGTGTGTGAACCT
GatcACAAGAACCCAGagcTACACCAACAGCTTTACCAGAGGCGTGTACTACCCCG
ACAAGGTGTTCAGATCCAGCGTGCTGCACTCTACCCAGGACCTGTTCCTGCCTTT
CTTCAGCAACGTGACCTGGTTCCACGCCATCTCCGGCACCAATGGCACCAAGAG
ATTCGACAACCCCGTGCTGCCCTTCAACGACGGGGTGTACTTTGCCAGCACCGAG
AAGTCCAACATCATCAGAGGCTGGATCTTCGGCACCACACTGGACAGCAAGACC
CAGAGCCTGCTGATCGTGAACAACGCCACCAACGTGGTCATCAAAGTGTGCGAG
TTCCAGTTCTGCAACGACCCCTTCCTGgacGTCTACTATCACAAGAACAACAAGAG
CTGGATGGAAAGCGAGTTCCGGGTGTACAGCAGCGCCAACAACTGCACCTTCGA
GTACGTGTCCCAGCCTTTCCTGATGGACCTGGAAGGCAAGCAGGGCAACTTCAA
GAACCTGCGCGAGTTCGTGTTCAAGAACATCGACGGCTACTTCAAGATCTACAGC
AAGCACACCCCTATCAACCTCggcCGGGATCTGCCTCAGGGCTTCTCTGCTCTGGA
ACCCCTGGTGGATCTGCCCATCGGCATCAACATCACCCGGTTTCAGACACTGCTG
GCCCTGCACAGAAGCTACCTGACACCTGGCGATAGCAGCAGCGGATGGACAGCT
GGTGCCGCCGCTTACTATGTGGGCTACCTGCAGCCTAGAACCTTTCTGCTGAAGT
ACAACGAGAACGGCACCATCACCGACGCCGTGGATTGTGCTCTGGATCCTCTGA
GCGAGACAAAGTGCACCCTGAAGTCCTTCACCGTGGAgAAGGGCATCTACCAGA
CCAGCAACTTCCGGGTGCAGCCCACCGAATCCATCGTGCGGTTCCCCAATATCAC
CAATCTGTGCCCCTTCgacGAGGTGTTCAATGCCACCAGATTCGCCTCTGTGTACG
CCTGGAACCGGAAGCGGATCAGCAATTGCGTGGCCGACTACTCCGTGCTGTACA
ACtccGCCagcTTCagcgccTTCAAGTGCTACGGCGTGTCCCCTACCAAGCTGAACGAC
CTGTGCTTCACAAACGTGTACGCCGACAGCTTCGTGATCCGGGGAaacGAAGTGagc
CAGATTGCCCCTGGACAGACAGGCaacATCGCCGACTACAACTACAAGCTGCCCG
ACGACTTCACCGGCTGTGTGATTGCCTGGAACAGCAACaagCTGGACTCCAAAGTC
ggcGGCAACTACAATTACcggTACCGGCTGTTCCGGAAGTCCAATCTGAAGCCCTTC
GAGCGGGACATCTCCACCGAGATCTATCAGGCCGGCaacaagCCTTGTAACGGCGT
GgccGGCgtgAACTGCTACTTCCCACTGcagTCCTACGGCTTTcggCCCACAtacGGCGT
GGGCcacCAGCCCTACAGAGTGGTGGTGCTGAGCTTCGAACTGCTGCATGCCCCTG
CCACAGTGTGCGGCCCTAAGAAgAGCACCAATCTCGTGAAGAACAAATGCGTGA
ACTTCAACTTCAACGGCCTGACCGGCACCGGCGTGCTGACAGAGAGCAACAAGA
AGTTCCTGCCATTCCAGCAGTTTGGCCGGGATATCGCCGATACCACAGACGCCGT
TAGAGATCCCCAGACACTGGAAATCCTGGACATCACCCCTTGCAGCTTCGGCGG
AGTGTCTGTGATCACCCCTGGCACCAACACCAGCAATCAGGTGGCAGTGCTGTAC
CAGggcGTGAACTGTACCGAAGTGCCCGTGGCCATTCACGCCGATCAGCTGACAC
CTACATGGCGGGTGTACTCCACCGGCAGCAATGTGTTTCAGACCAGAGCCGGCT
GTCTGATCGGAGCCGAGtacGTGAACAATAGCTACGAGTGCGACATCCCCATCGG
CGCTGGCATCTGTGCCAGCTACCAGACACAGACAaagAGCcacGCCTCTGTGGCCA
GCCAGAGCATCATTGCCTACACAATGTCTCTGGGCGCCGAGAACAGCGTGGCCT
ACTCCAACAACTCTATCGCTATCCCCACCAACTTCACCATCAGCGTGACCACAGA
GATCCTGCCTGTGTCCATGACCAAGACCAGCGTGGACTGCACCATGTACATCTGC
GGCGATTCCACCGAGTGCTCCAACCTGCTGCTGCAGTACGGCAGCTTCTGCACCC
AGCTGaagAGAGCCCTGACAGGGATCGCCGTGGAACAGGACAAGAACACCCAAG
AGGTGTTCGCCCAAGTGAAGCAGATCTACAAGACCCCTCCTATCAAGtacTTCGGC
GGCTTCAATTTCAGCCAGATTCTGCCCGATCCTAGCAAGCCCAGCAAGCGGAGCc
ctATCGAGGACCTGCTGTTCAACAAAGTGACACTGGCCGACGCCGGCTTCATCAA
GCAGTATGGCGATTGTCTGGGCGACATTGCCGCCAGGGATCTGATTTGCGCCCAG
AAGTTTAACGGACTGACAGTGCTGCCTCCTCTGCTGACCGATGAGATGATCGCCC
AGTACACATCTGCCCTGCTGGCCGGCACAATCACAAGCGGCTGGACATTTGGAG
CTGGCcctGCTCTGCAGATCCCCTTTccaATGCAGATGGCCTACCGGTTCAACGGCA
TCGGAGTGACCCAGAATGTGCTGTACGAGAACCAGAAGCTGATCGCCAACCAGT
TCAACAGCGCCATCGGCAAGATCCAGGACAGCCTGAGCAGCACAcccAGCGCCCT
GGGAAAGCTGCAGGACGTGGTCAACcacAATGCCCAGGCACTGAACACCCTGGTC
AAGCAGCTGTCCTCCaagTTCGGCGCCATCAGCTCTGTGCTGAACGATATCCTGAG
CAGACTGGACccccctGAAGCCGAGGTGCAGATCGACAGACTGATCACCGGAAGGC
TGCAGTCCCTGCAGACCTACGTTACCCAGCAGCTGATCAGAGCCGCCGAGATTA
GAGCCTCTGCCAATCTGGCCGCCACCAAGATGTCTGAGTGTGTGCTGGGCCAGA
GCAAGAGAGTGGACTTTTGCGGCAAGGGCTACCACCTGATGAGCTTCCCTCAGTC
TGCCCCTCACGGCGTGGTGTTTCTGCACGTGACATACGTGCCCGCTCAAGAGAAG
AATTTCACCACCGCTCCAGCCATCTGCCACGACGGCAAAGCCCACTTTCCTAGAG
AAGGCGTGTTCGTGTCCAACGGCACCCATTGGTTCGTGACCCAGCGGAACTTCTA
CGAGCCCCAGATCATCACCACCGACAACACCTTCGTGTCTGGCAACTGCGACGTC
GTGATCGGCATTGTGAACAATACCGTGTACGACCCTCTGCAGCCCGAGCTGGAC
AGCTTCAAAGAGGAACTGGATAAGTACTTTAAGAACCACACAAGCCCCGACGTG
GACCTGGGCGATATCAGCGGAATCAATGCCAGCGTCGTGAACATCCAGAAAGAG
ATCGACCGGCTGAACGAGGTGGCCAAGAATCTGAACGAGAGCCTGATCGACCTG
CAAGAACTGGGGAAGTACGAGCAGTACATCAAGTGGCCCggcggaggtgggtcgCTCAT
AACATACATCGTCCTGACTATAATCAGCTTGGTATTTGGTATTTTGTCTTTGA
TTCTTGCATGCTATTTGATGTATAAACAGAAAGCTCAGCAGAAGACTCTCCT
GTGGCTCGGTAACAACACACTCGACCAGATGAGAGCAACTACAAAGATGTG
A
Amino Acid Sequence of BA.4/5 S371 S373 S375 (Signal peptide is in bold and
underlined; GS linker is in italics; TM and CT From F is in bold and double
underlined; Spike ectodomain is underlined; S371 S373 S375 is in bold, underlined
and italics): SEQ ID NO: 67
MFVFLVLLPLVSSQCVNLITRTQSYTNSFTRGVYYPDKVFRSSVLHSTQDLFLPFFSN
VTWFHAISGTNGTKRFDNPVLPFNDGVYFASTEKSNIIRGWIFGTTLDSKTQSLLIVN
NATNVVIKVCEFQFCNDPFLDVYYHKNNKSWMESEFRVYSSANNCTFEYVSQPFLM
DLEGKQGNFKNLREFVFKNIDGYFKIYSKHTPINLGRDLPQGFSALEPLVDLPIGINIT
RFQTLLALHRSYLTPGDSSSGWTAGAAAYYVGYLQPRTFLLKYNENGTITDAVDCA
LDPLSETKCTLKSFTVEKGIYQTSNFRVQPTESIVRFPNITNLCPFDEVFNATRFASVY
AWNRKRISNCVADYSVLYNSASFSAFKCYGVSPTKLNDLCFTNVYADSFVIRGNEVS
QIAPGQTGNIADYNYKLPDDFTGCVIAWNSNKLDSKVGGNYNYRYRLFRKSNLKPF
ERDISTEIYQAGNKPCNGVAGVNCYFPLQSYGFRPTYGVGHQPYRVVVLSFELLHAP
ATVCGPKKSTNLVKNKCVNFNFNGLTGTGVLTESNKKFLPFQQFGRDIADTTDAVR
DPQTLEILDITPCSFGGVSVITPGTNTSNQVAVLYQGVNCTEVPVAIHADQLTPTWRV
YSTGSNVFQTRAGCLIGAEYVNNSYECDIPIGAGICASYQTQTKSHASVASQSIIAYT
MSLGAENSVAYSNNSIAIPTNFTISVTTEILPVSMTKTSVDCTMYICGDSTECSNLLLQ
YGSFCTQLKRALTGIAVEQDKNTQEVFAQVKQIYKTPPIKYFGGFNFSQILPDPSKPS
KRSPIEDLLFNKVTLADAGFIKQYGDCLGDIAARDLICAQKFNGLTVLPPLLTDEMIA
QYTSALLAGTITSGWTFGAGPALQIPFPMQMAYRENGIGVTQNVLYENQKLIANQFN
SAIGKIQDSLSSTPSALGKLQDVVNHNAQALNTLVKQLSSKFGAISSVLNDILSRLDPP
EAEVQIDRLITGRLQSLQTYVTQQLIRAAEIRASANLAATKMSECVLGQSKRVDFCG
KGYHLMSFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKEELDKYFKN
HTSPDVDLGDISGINASVVNIQKEIDRLNEVAKNLNESLIDLQELGKYEQYIKWPGGG
GSLITYIVLTIISLVFGILSLILACYLMYKQKAQQKTLLWLGNNTLDQMRATTK
M*
Nucleotide Sequence of BA.4/5 S371 S373 S375 (Signal peptide is in bold and
underlined; Spike ectodomain is underlined; S371 S373 S375 is in bold, underlined
and italics): SEQ ID NO: 68
ATGTTCGTGTTTCTGGTGCTGCTGCCTCTGGTGTCCAGCCAGTGTGTGAACCT
GatcACAAGAACCCAGagcTACACCAACAGCTTTACCAGAGGCGTGTACTACCCCG
ACAAGGTGTTCAGATCCAGCGTGCTGCACTCTACCCAGGACCTGTTCCTGCCTTT
CTTCAGCAACGTGACCTGGTTCCACGCCATCTCCGGCACCAATGGCACCAAGAG
ATTCGACAACCCCGTGCTGCCCTTCAACGACGGGGTGTACTTTGCCAGCACCGAG
AAGTCCAACATCATCAGAGGCTGGATCTTCGGCACCACACTGGACAGCAAGACC
CAGAGCCTGCTGATCGTGAACAACGCCACCAACGTGGTCATCAAAGTGTGCGAG
TTCCAGTTCTGCAACGACCCCTTCCTGgacGTCTACTATCACAAGAACAACAAGAG
CTGGATGGAAAGCGAGTTCCGGGTGTACAGCAGCGCCAACAACTGCACCTTCGA
GTACGTGTCCCAGCCTTTCCTGATGGACCTGGAAGGCAAGCAGGGCAACTTCAA
GAACCTGCGCGAGTTCGTGTTCAAGAACATCGACGGCTACTTCAAGATCTACAGC
AAGCACACCCCTATCAACCTCggcCGGGATCTGCCTCAGGGCTTCTCTGCTCTGGA
ACCCCTGGTGGATCTGCCCATCGGCATCAACATCACCCGGTTTCAGACACTGCTG
GCCCTGCACAGAAGCTACCTGACACCTGGCGATAGCAGCAGCGGATGGACAGCT
GGTGCCGCCGCTTACTATGTGGGCTACCTGCAGCCTAGAACCTTTCTGCTGAAGT
ACAACGAGAACGGCACCATCACCGACGCCGTGGATTGTGCTCTGGATCCTCTGA
GCGAGACAAAGTGCACCCTGAAGTCCTTCACCGTGGAgAAGGGCATCTACCAGA
CCAGCAACTTCCGGGTGCAGCCCACCGAATCCATCGTGCGGTTCCCCAATATCAC
CAATCTGTGCCCCTTCgacGAGGTGTTCAATGCCACCAGATTCGCCTCTGTGTACG
CCTGGAACCGGAAGCGGATCAGCAATTGCGTGGCCGACTACTCCGTGCTGTACA
ACtccGCCagcTTCagcgccTTCAAGTGCTACGGCGTGTCCCCTACCAAGCTGAACGAC
CTGTGCTTCACAAACGTGTACGCCGACAGCTTCGTGATCCGGGGAaacGAAGTGagc
CAGATTGCCCCTGGACAGACAGGCaacATCGCCGACTACAACTACAAGCTGCCCG
ACGACTTCACCGGCTGTGTGATTGCCTGGAACAGCAACaagCTGGACTCCAAAGTC
ggcGGCAACTACAATTACcggTACCGGCTGTTCCGGAAGTCCAATCTGAAGCCCTTC
GAGCGGGACATCTCCACCGAGATCTATCAGGCCGGCaacaagCCTTGTAACGGCGT
GgccGGCgtgAACTGCTACTTCCCACTGcagTCCTACGGCTTTcggCCCACAtacGGCGT
GGGCcacCAGCCCTACAGAGTGGTGGTGCTGAGCTTCGAACTGCTGCATGCCCCTG
CCACAGTGTGCGGCCCTAAGAAgAGCACCAATCTCGTGAAGAACAAATGCGTGA
ACTTCAACTTCAACGGCCTGACCGGCACCGGCGTGCTGACAGAGAGCAACAAGA
AGTTCCTGCCATTCCAGCAGTTTGGCCGGGATATCGCCGATACCACAGACGCCGT
TAGAGATCCCCAGACACTGGAAATCCTGGACATCACCCCTTGCAGCTTCGGCGG
AGTGTCTGTGATCACCCCTGGCACCAACACCAGCAATCAGGTGGCAGTGCTGTAC
CAGggcGTGAACTGTACCGAAGTGCCCGTGGCCATTCACGCCGATCAGCTGACAC
CTACATGGCGGGTGTACTCCACCGGCAGCAATGTGTTTCAGACCAGAGCCGGCT
GTCTGATCGGAGCCGAGtacGTGAACAATAGCTACGAGTGCGACATCCCCATCGG
CGCTGGCATCTGTGCCAGCTACCAGACACAGACAaagAGCcacGCCTCTGTGGCCA
GCCAGAGCATCATTGCCTACACAATGTCTCTGGGCGCCGAGAACAGCGTGGCCT
ACTCCAACAACTCTATCGCTATCCCCACCAACTTCACCATCAGCGTGACCACAGA
GATCCTGCCTGTGTCCATGACCAAGACCAGCGTGGACTGCACCATGTACATCTGC
GGCGATTCCACCGAGTGCTCCAACCTGCTGCTGCAGTACGGCAGCTTCTGCACCC
AGCTGaagAGAGCCCTGACAGGGATCGCCGTGGAACAGGACAAGAACACCCAAG
AGGTGTTCGCCCAAGTGAAGCAGATCTACAAGACCCCTCCTATCAAGtacTTCGGC
GGCTTCAATTTCAGCCAGATTCTGCCCGATCCTAGCAAGCCCAGCAAGCGGAGCc
ctATCGAGGACCTGCTGTTCAACAAAGTGACACTGGCCGACGCCGGCTTCATCAA
GCAGTATGGCGATTGTCTGGGCGACATTGCCGCCAGGGATCTGATTTGCGCCCAG
AAGTTTAACGGACTGACAGTGCTGCCTCCTCTGCTGACCGATGAGATGATCGCCC
AGTACACATCTGCCCTGCTGGCCGGCACAATCACAAGCGGCTGGACATTTGGAG
CTGGCcctGCTCTGCAGATCCCCTTTccaATGCAGATGGCCTACCGGTTCAACGGCA
TCGGAGTGACCCAGAATGTGCTGTACGAGAACCAGAAGCTGATCGCCAACCAGT
TCAACAGCGCCATCGGCAAGATCCAGGACAGCCTGAGCAGCACAcccAGCGCCCT
GGGAAAGCTGCAGGACGTGGTCAACcacAATGCCCAGGCACTGAACACCCTGGTC
AAGCAGCTGTCCTCCaagTTCGGCGCCATCAGCTCTGTGCTGAACGATATCCTGAG
CAGACTGGACccccctGAAGCCGAGGTGCAGATCGACAGACTGATCACCGGAAGGC
TGCAGTCCCTGCAGACCTACGTTACCCAGCAGCTGATCAGAGCCGCCGAGATTA
GAGCCTCTGCCAATCTGGCCGCCACCAAGATGTCTGAGTGTGTGCTGGGCCAGA
GCAAGAGAGTGGACTTTTGCGGCAAGGGCTACCACCTGATGAGCTTCCCTCAGTC
TGCCCCTCACGGCGTGGTGTTTCTGCACGTGACATACGTGCCCGCTCAAGAGAAG
AATTTCACCACCGCTCCAGCCATCTGCCACGACGGCAAAGCCCACTTTCCTAGAG
AAGGCGTGTTCGTGTCCAACGGCACCCATTGGTTCGTGACCCAGCGGAACTTCTA
CGAGCCCCAGATCATCACCACCGACAACACCTTCGTGTCTGGCAACTGCGACGTC
GTGATCGGCATTGTGAACAATACCGTGTACGACCCTCTGCAGCCCGAGCTGGAC
AGCTTCAAAGAGGAACTGGATAAGTACTTTAAGAACCACACAAGCCCCGACGTG
GACCTGGGCGATATCAGCGGAATCAATGCCAGCGTCGTGAACATCCAGAAAGAG
ATCGACCGGCTGAACGAGGTGGCCAAGAATCTGAACGAGAGCCTGATCGACCTG
CAAGAACTGGGGAAGTACGAGCAGTACATCAAGTGGCCC
Amino Acid Sequence of BA.4/5 S371 S373 S375 (Signal peptide is in bold and
underlined; Spike ectodomain is underlined; S371 S373 S375 is in bold, underlined
and italics): SEQ ID NO: 69
MFVFLVLLPLVSSQCVNLITRTQSYTNSFTRGVYYPDKVFRSSVLHSTQDLFLPFFSN
VTWFHAISGTNGTKRFDNPVLPFNDGVYFASTEKSNIIRGWIFGTTLDSKTQSLLIVN
NATNVVIKVCEFQFCNDPFLDVYYHKNNKSWMESEFRVYSSANNCTFEYVSQPFLM
DLEGKQGNFKNLREFVFKNIDGYFKIYSKHTPINLGRDLPQGFSALEPLVDLPIGINIT
RFQTLLALHRSYLTPGDSSSGWTAGAAAYYVGYLQPRTFLLKYNENGTITDAVDCA
LDPLSETKCTLKSFTVEKGIYQTSNFRVQPTESIVRFPNITNLCPFDEVFNATRFASVY
AWNRKRISNCVADYSVLYNSASFSAFKCYGVSPTKLNDLCFTNVYADSFVIRGNEVS
QIAPGQTGNIADYNYKLPDDFTGCVIAWNSNKLDSKVGGNYNYRYRLFRKSNLKPF
ERDISTEIYQAGNKPCNGVAGVNCYFPLQSYGFRPTYGVGHQPYRVVVLSFELLHAP
ATVCGPKKSTNLVKNKCVNFNFNGLTGTGVLTESNKKFLPFQQFGRDIADTTDAVR
DPQTLEILDITPCSFGGVSVITPGTNTSNQVAVLYQGVNCTEVPVAIHADQLTPTWRV
YSTGSNVFQTRAGCLIGAEYVNNSYECDIPIGAGICASYQTQTKSHASVASQSIIAYT
MSLGAENSVAYSNNSIAIPTNFTISVTTEILPVSMTKTSVDCTMYICGDSTECSNLLLQ
YGSFCTQLKRALTGIAVEQDKNTQEVFAQVKQIYKTPPIKYFGGFNFSQILPDPSKPS
KRSPIEDLLFNKVTLADAGFIKQYGDCLGDIAARDLICAQKFNGLTVLPPLLTDEMIA
QYTSALLAGTITSGWTFGAGPALQIPFPMQMAYRENGIGVTQNVLYENQKLIANQFN
SAIGKIQDSLSSTPSALGKLQDVVNHNAQALNTLVKQLSSKFGAISSVLNDILSRLDPP
EAEVQIDRLITGRLQSLQTYVTQQLIRAAEIRASANLAATKMSECVLGQSKRVDFCG
KGYHLMSFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKEELDKYFKN
HTSPDVDLGDISGINASVVNIQKEIDRLNEVAKNLNESLIDLQELGKYEQYIKWP
Nucleotide Sequence of BA.4/5 S371 S373 S375 (Spike ectodomain without signal
peptide is underlined; S371 S373 S375 is in bold, underlined and italics): SEQ ID
NO: 70
CAGTGTGTGAACCTGatcACAAGAACCCAGagcTACACCAACAGCTTTACCAGAGG
CGTGTACTACCCCGACAAGGTGTTCAGATCCAGCGTGCTGCACTCTACCCAGGAC
CTGTTCCTGCCTTTCTTCAGCAACGTGACCTGGTTCCACGCCATCTCCGGCACCAA
TGGCACCAAGAGATTCGACAACCCCGTGCTGCCCTTCAACGACGGGGTGTACTTT
GCCAGCACCGAGAAGTCCAACATCATCAGAGGCTGGATCTTCGGCACCACACTG
GACAGCAAGACCCAGAGCCTGCTGATCGTGAACAACGCCACCAACGTGGTCATC
AAAGTGTGCGAGTTCCAGTTCTGCAACGACCCCTTCCTGgacGTCTACTATCACAA
GAACAACAAGAGCTGGATGGAAAGCGAGTTCCGGGTGTACAGCAGCGCCAACA
ACTGCACCTTCGAGTACGTGTCCCAGCCTTTCCTGATGGACCTGGAAGGCAAGCA
GGGCAACTTCAAGAACCTGCGCGAGTTCGTGTTCAAGAACATCGACGGCTACTTC
AAGATCTACAGCAAGCACACCCCTATCAACCTCggcCGGGATCTGCCTCAGGGCTT
CTCTGCTCTGGAACCCCTGGTGGATCTGCCCATCGGCATCAACATCACCCGGTTT
CAGACACTGCTGGCCCTGCACAGAAGCTACCTGACACCTGGCGATAGCAGCAGC
GGATGGACAGCTGGTGCCGCCGCTTACTATGTGGGCTACCTGCAGCCTAGAACCT
TTCTGCTGAAGTACAACGAGAACGGCACCATCACCGACGCCGTGGATTGTGCTCT
GGATCCTCTGAGCGAGACAAAGTGCACCCTGAAGTCCTTCACCGTGGAgAAGGG
CATCTACCAGACCAGCAACTTCCGGGTGCAGCCCACCGAATCCATCGTGCGGTTC
CCCAATATCACCAATCTGTGCCCCTTCgacGAGGTGTTCAATGCCACCAGATTCGC
CTCTGTGTACGCCTGGAACCGGAAGCGGATCAGCAATTGCGTGGCCGACTACTCC
GTGCTGTACAACtccGCCagcTTCagcgccTTCAAGTGCTACGGCGTGTCCCCTACCAA
GCTGAACGACCTGTGCTTCACAAACGTGTACGCCGACAGCTTCGTGATCCGGGG
AaacGAAGTGagcCAGATTGCCCCTGGACAGACAGGCaacATCGCCGACTACAACTA
CAAGCTGCCCGACGACTTCACCGGCTGTGTGATTGCCTGGAACAGCAACaagCTG
GACTCCAAAGTCggcGGCAACTACAATTACcggTACCGGCTGTTCCGGAAGTCCAA
TCTGAAGCCCTTCGAGCGGGACATCTCCACCGAGATCTATCAGGCCGGCaacaagCC
TTGTAACGGCGTGgccGGCgtgAACTGCTACTTCCCACTGcagTCCTACGGCTTTcggC
CCACAtacGGCGTGGGCcacCAGCCCTACAGAGTGGTGGTGCTGAGCTTCGAACTGC
TGCATGCCCCTGCCACAGTGTGCGGCCCTAAGAAgAGCACCAATCTCGTGAAGAA
CAAATGCGTGAACTTCAACTTCAACGGCCTGACCGGCACCGGCGTGCTGACAGA
GAGCAACAAGAAGTTCCTGCCATTCCAGCAGTTTGGCCGGGATATCGCCGATAC
CACAGACGCCGTTAGAGATCCCCAGACACTGGAAATCCTGGACATCACCCCTTG
CAGCTTCGGCGGAGTGTCTGTGATCACCCCTGGCACCAACACCAGCAATCAGGT
GGCAGTGCTGTACCAGggcGTGAACTGTACCGAAGTGCCCGTGGCCATTCACGCC
GATCAGCTGACACCTACATGGCGGGTGTACTCCACCGGCAGCAATGTGTTTCAGA
CCAGAGCCGGCTGTCTGATCGGAGCCGAGtacGTGAACAATAGCTACGAGTGCGA
CATCCCCATCGGCGCTGGCATCTGTGCCAGCTACCAGACACAGACAaagAGCcacG
CCTCTGTGGCCAGCCAGAGCATCATTGCCTACACAATGTCTCTGGGCGCCGAGAA
CAGCGTGGCCTACTCCAACAACTCTATCGCTATCCCCACCAACTTCACCATCAGC
GTGACCACAGAGATCCTGCCTGTGTCCATGACCAAGACCAGCGTGGACTGCACC
ATGTACATCTGCGGCGATTCCACCGAGTGCTCCAACCTGCTGCTGCAGTACGGCA
GCTTCTGCACCCAGCTGaagAGAGCCCTGACAGGGATCGCCGTGGAACAGGACAA
GAACACCCAAGAGGTGTTCGCCCAAGTGAAGCAGATCTACAAGACCCCTCCTAT
CAAGtacTTCGGCGGCTTCAATTTCAGCCAGATTCTGCCCGATCCTAGCAAGCCCA
GCAAGCGGAGCcctATCGAGGACCTGCTGTTCAACAAAGTGACACTGGCCGACGC
CGGCTTCATCAAGCAGTATGGCGATTGTCTGGGCGACATTGCCGCCAGGGATCTG
ATTTGCGCCCAGAAGTTTAACGGACTGACAGTGCTGCCTCCTCTGCTGACCGATG
AGATGATCGCCCAGTACACATCTGCCCTGCTGGCCGGCACAATCACAAGCGGCT
GGACATTTGGAGCTGGCcctGCTCTGCAGATCCCCTTTccaATGCAGATGGCCTACC
GGTTCAACGGCATCGGAGTGACCCAGAATGTGCTGTACGAGAACCAGAAGCTGA
TCGCCAACCAGTTCAACAGCGCCATCGGCAAGATCCAGGACAGCCTGAGCAGCA
CAcccAGCGCCCTGGGAAAGCTGCAGGACGTGGTCAACcacAATGCCCAGGCACTG
AACACCCTGGTCAAGCAGCTGTCCTCCaagTTCGGCGCCATCAGCTCTGTGCTGAA
CGATATCCTGAGCAGACTGGACccccctGAAGCCGAGGTGCAGATCGACAGACTGA
TCACCGGAAGGCTGCAGTCCCTGCAGACCTACGTTACCCAGCAGCTGATCAGAG
CCGCCGAGATTAGAGCCTCTGCCAATCTGGCCGCCACCAAGATGTCTGAGTGTGT
GCTGGGCCAGAGCAAGAGAGTGGACTTTTGCGGCAAGGGCTACCACCTGATGAG
CTTCCCTCAGTCTGCCCCTCACGGCGTGGTGTTTCTGCACGTGACATACGTGCCC
GCTCAAGAGAAGAATTTCACCACCGCTCCAGCCATCTGCCACGACGGCAAAGCC
CACTTTCCTAGAGAAGGCGTGTTCGTGTCCAACGGCACCCATTGGTTCGTGACCC
AGCGGAACTTCTACGAGCCCCAGATCATCACCACCGACAACACCTTCGTGTCTGG
CAACTGCGACGTCGTGATCGGCATTGTGAACAATACCGTGTACGACCCTCTGCAG
CCCGAGCTGGACAGCTTCAAAGAGGAACTGGATAAGTACTTTAAGAACCACACA
AGCCCCGACGTGGACCTGGGCGATATCAGCGGAATCAATGCCAGCGTCGTGAAC
ATCCAGAAAGAGATCGACCGGCTGAACGAGGTGGCCAAGAATCTGAACGAGAG
CCTGATCGACCTGCAAGAACTGGGGAAGTACGAGCAGTACATCAAGTGGCCC
Amino Acid Sequence of BA.4/5 S371 S373 S375 (Spike ectodomain without signal
peptide is underlined; S371 S373 S375 is in bold, underlined and italics): SEQ ID
NO: 71
QCVNLITRTQSYTNSFTRGVYYPDKVFRSSVLHSTQDLFLPFFSNVTWFHAISGTNGT
KRFDNPVLPFNDGVYFASTEKSNIIRGWIFGTTLDSKTQSLLIVNNATNVVIKVCEFQF
CNDPFLDVYYHKNNKSWMESEFRVYSSANNCTFEYVSQPFLMDLEGKQGNFKNLR
EFVFKNIDGYFKIYSKHTPINLGRDLPQGFSALEPLVDLPIGINITRFQTLLALHRSYLT
PGDSSSGWTAGAAAYYVGYLQPRTFLLKYNENGTITDAVDCALDPLSETKCTLKSFT
VEKGIYQTSNERVQPTESIVRFPNITNLCPFDEVFNATRFASVYAWNRKRISNCVADY
SVLYNSASFSAFKCYGVSPTKLNDLCFTNVYADSFVIRGNEVSQIAPGQTGNIADYN
YKLPDDFTGCVIAWNSNKLDSKVGGNYNYRYRLFRKSNLKPFERDISTEIYQAGNKP
CNGVAGVNCYFPLQSYGFRPTYGVGHQPYRVVVLSFELLHAPATVCGPKKSTNLVK
NKCVNFNFNGLTGTGVLTESNKKFLPFQQFGRDIADTTDAVRDPQTLEILDITPCSFG
GVSVITPGTNTSNQVAVLYQGVNCTEVPVAIHADQLTPTWRVYSTGSNVFQTRAGC
LIGAEYVNNSYECDIPIGAGICASYQTQTKSHASVASQSIIAYTMSLGAENSVAYSNN
SIAIPTNFTISVTTEILPVSMTKTSVDCTMYICGDSTECSNLLLQYGSFCTQLKRALTGI
AVEQDKNTQEVFAQVKQIYKTPPIKYFGGFNFSQILPDPSKPSKRSPIEDLLFNKVTLA
DAGFIKQYGDCLGDIAARDLICAQKFNGLTVLPPLLTDEMIAQYTSALLAGTITSGWT
FGAGPALQIPFPMQMAYRENGIGVTQNVLYENQKLIANQFNSAIGKIQDSLSSTPSAL
GKLQDVVNHNAQALNTLVKQLSSKFGAISSVLNDILSRLDPPEAEVQIDRLITGRLQS
LQTYVTQQLIRAAEIRASANLAATKMSECVLGQSKRVDFCGKGYHLMSFPQSAPHG
VVFLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISGINA
SVVNIQKEIDRLNEVAKNLNESLIDLQELGKYEQYIKWP
Nucleotide Sequence of BA.5 (Signal peptide is in bold and underlined; GS linker
is in italics; TM and CT From F is in bold and double underlined; Spike ectodomain
is underlined): SEQ ID NO: 74
ATGTTCGTGTTTCTGGTGCTGCTGCCTCTGGTGTCCAGCCAGTGTGTGAACCT
GatcACAAGAACCCAGagcTACACCAACAGCTTTACCAGAGGCGTGTACTACCCCG
ACAAGGTGTTCAGATCCAGCGTGCTGCACTCTACCCAGGACCTGTTCCTGCCTTT
CTTCAGCAACGTGACCTGGTTCCACGCCATCTCCGGCACCAATGGCACCAAGAG
ATTCGACAACCCCGTGCTGCCCTTCAACGACGGGGTGTACTTTGCCAGCACCGAG
AAGTCCAACATCATCAGAGGCTGGATCTTCGGCACCACACTGGACAGCAAGACC
CAGAGCCTGCTGATCGTGAACAACGCCACCAACGTGGTCATCAAAGTGTGCGAG
TTCCAGTTCTGCAACGACCCCTTCCTGgacGTCTACTATCACAAGAACAACAAGAG
CTGGATGGAAAGCGAGTTCCGGGTGTACAGCAGCGCCAACAACTGCACCTTCGA
GTACGTGTCCCAGCCTTTCCTGATGGACCTGGAAGGCAAGCAGGGCAACTTCAA
GAACCTGCGCGAGTTCGTGTTCAAGAACATCGACGGCTACTTCAAGATCTACAGC
AAGCACACCCCTATCAACCTCggcCGGGATCTGCCTCAGGGCTTCTCTGCTCTGGA
ACCCCTGGTGGATCTGCCCATCGGCATCAACATCACCCGGTTTCAGACACTGCTG
GCCCTGCACAGAAGCTACCTGACACCTGGCGATAGCAGCAGCGGATGGACAGCT
GGTGCCGCCGCTTACTATGTGGGCTACCTGCAGCCTAGAACCTTTCTGCTGAAGT
ACAACGAGAACGGCACCATCACCGACGCCGTGGATTGTGCTCTGGATCCTCTGA
GCGAGACAAAGTGCACCCTGAAGTCCTTCACCGTGGAgAAGGGCATCTACCAGA
CCAGCAACTTCCGGGTGCAGCCCACCGAATCCATCGTGCGGTTCCCCAATATCAC
CAATCTGTGCCCCTTCgacGAGGTGTTCAATGCCACCAGATTCGCCTCTGTGTACG
CCTGGAACCGGAAGCGGATCAGCAATTGCGTGGCCGACTACTCCGTGCTGTACA
ACttcGCCcctTTCttcgccTTCAAGTGCTACGGCGTGTCCCCTACCAAGCTGAACGACC
TGTGCTTCACAAACGTGTACGCCGACAGCTTCGTGATCCGGGGAaacGAAGTGagcC
AGATTGCCCCTGGACAGACAGGCaacATCGCCGACTACAACTACAAGCTGCCCGA
CGACTTCACCGGCTGTGTGATTGCCTGGAACAGCAACaagCTGGACTCCAAAGTCa
gcGGCAACTACAATTACcggTACCGGCTGTTCCGGAAGTCCAATCTGAAGCCCTTC
GAGCGGGACATCTCCACCGAGATCTATCAGGCCGGCaacaagCCTTGTAACGGCGT
GgccGGCgtgAACTGCTACTTCCCACTGcagTCCTACGGCTTTcggCCCACAtacGGCGT
GGGCcacCAGCCCTACAGAGTGGTGGTGCTGAGCTTCGAACTGCTGCATGCCCCTG
CCACAGTGTGCGGCCCTAAGAAgAGCACCAATCTCGTGAAGAACAAATGCGTGA
ACTTCAACTTCAACGGCCTGACCGGCACCGGCGTGCTGACAGAGAGCAACAAGA
AGTTCCTGCCATTCCAGCAGTTTGGCCGGGATATCGCCGATACCACAGACGCCGT
TAGAGATCCCCAGACACTGGAAATCCTGGACATCACCCCTTGCAGCTTCGGCGG
AGTGTCTGTGATCACCCCTGGCACCAACACCAGCAATCAGGTGGCAGTGCTGTAC
CAGggcGTGAACTGTACCGAAGTGCCCGTGGCCATTCACGCCGATCAGCTGACAC
CTACATGGCGGGTGTACTCCACCGGCAGCAATGTGTTTCAGACCAGAGCCGGCT
GTCTGATCGGAGCCGAGtacGTGAACAATAGCTACGAGTGCGACATCCCCATCGG
CGCTGGCATCTGTGCCAGCTACCAGACACAGACAaagAGCcacGCCTCTGTGGCCA
GCCAGAGCATCATTGCCTACACAATGTCTCTGGGCGCCGAGAACAGCGTGGCCT
ACTCCAACAACTCTATCGCTATCCCCACCAACTTCACCATCAGCGTGACCACAGA
GATCCTGCCTGTGTCCATGACCAAGACCAGCGTGGACTGCACCATGTACATCTGC
GGCGATTCCACCGAGTGCTCCAACCTGCTGCTGCAGTACGGCAGCTTCTGCACCC
AGCTGaagAGAGCCCTGACAGGGATCGCCGTGGAACAGGACAAGAACACCCAAG
AGGTGTTCGCCCAAGTGAAGCAGATCTACAAGACCCCTCCTATCAAGtacTTCGGC
GGCTTCAATTTCAGCCAGATTCTGCCCGATCCTAGCAAGCCCAGCAAGCGGAGCc
ctATCGAGGACCTGCTGTTCAACAAAGTGACACTGGCCGACGCCGGCTTCATCAA
GCAGTATGGCGATTGTCTGGGCGACATTGCCGCCAGGGATCTGATTTGCGCCCAG
AAGTTTAACGGACTGACAGTGCTGCCTCCTCTGCTGACCGATGAGATGATCGCCC
AGTACACATCTGCCCTGCTGGCCGGCACAATCACAAGCGGCTGGACATTTGGAG
CTGGCcctGCTCTGCAGATCCCCTTTccaATGCAGATGGCCTACCGGTTCAACGGCA
TCGGAGTGACCCAGAATGTGCTGTACGAGAACCAGAAGCTGATCGCCAACCAGT
TCAACAGCGCCATCGGCAAGATCCAGGACAGCCTGAGCAGCACAcccAGCGCCCT
GGGAAAGCTGCAGGACGTGGTCAACcacAATGCCCAGGCACTGAACACCCTGGTC
AAGCAGCTGTCCTCCaagTTCGGCGCCATCAGCTCTGTGCTGAACGATATCCTGAG
CAGACTGGACccccctGAAGCCGAGGTGCAGATCGACAGACTGATCACCGGAAGGC
TGCAGTCCCTGCAGACCTACGTTACCCAGCAGCTGATCAGAGCCGCCGAGATTA
GAGCCTCTGCCAATCTGGCCGCCACCAAGATGTCTGAGTGTGTGCTGGGCCAGA
GCAAGAGAGTGGACTTTTGCGGCAAGGGCTACCACCTGATGAGCTTCCCTCAGTC
TGCCCCTCACGGCGTGGTGTTTCTGCACGTGACATACGTGCCCGCTCAAGAGAAG
AATTTCACCACCGCTCCAGCCATCTGCCACGACGGCAAAGCCCACTTTCCTAGAG
AAGGCGTGTTCGTGTCCAACGGCACCCATTGGTTCGTGACCCAGCGGAACTTCTA
CGAGCCCCAGATCATCACCACCGACAACACCTTCGTGTCTGGCAACTGCGACGTC
GTGATCGGCATTGTGAACAATACCGTGTACGACCCTCTGCAGCCCGAGCTGGAC
AGCTTCAAAGAGGAACTGGATAAGTACTTTAAGAACCACACAAGCCCCGACGTG
GACCTGGGCGATATCAGCGGAATCAATGCCAGCGTCGTGAACATCCAGAAAGAG
ATCGACCGGCTGAACGAGGTGGCCAAGAATCTGAACGAGAGCCTGATCGACCTG
CAAGAACTGGGGAAGTACGAGCAGTACATCAAGTGGCCCggcggaggtgggtcgCTCAT
AACATACATCGTCCTGACTATAATCAGCTTGGTATTTGGTATTTTGTCTTTGA
TTCTTGCATGCTATTTGATGTATAAACAGAAAGCTCAGCAGAAGACTCTCCT
GTGGCTCGGTAACAACACACTCGACCAGATGAGAGCAACTACAAAGATGTG
ATAA
Amino Acid Sequence of BA.5 (Signal peptide is in bold and underlined; GS linker
is in italics; TM and CT From F is in bold and double underlined; Spike ectodomain
is underlined): SEQ ID NO: 75
MFVFLVLLPLVSSQCVNLITRTQSYTNSFTRGVYYPDKVFRSSVLHSTQDLFLPFFSN
VTWFHAISGTNGTKRFDNPVLPFNDGVYFASTEKSNIIRGWIFGTTLDSKTQSLLIVN
NATNVVIKVCEFQFCNDPFLDVYYHKNNKSWMESEFRVYSSANNCTFEYVSQPFLM
DLEGKQGNFKNLREFVFKNIDGYFKIYSKHTPINLGRDLPQGFSALEPLVDLPIGINIT
RFQTLLALHRSYLTPGDSSSGWTAGAAAYYVGYLQPRTFLLKYNENGTITDAVDCA
LDPLSETKCTLKSFTVEKGIYQTSNFRVQPTESIVRFPNITNLCPFDEVFNATRFASVY
AWNRKRISNCVADYSVLYNFAPFFAFKCYGVSPTKLNDLCFTNVYADSFVIRGNEVS
QIAPGQTGNIADYNYKLPDDFTGCVIAWNSNKLDSKVSGNYNYRYRLFRKSNLKPFE
RDISTEIYQAGNKPCNGVAGVNCYFPLQSYGFRPTYGVGHQPYRVVVLSFELLHAPA
TVCGPKKSTNLVKNKCVNFNFNGLTGTGVLTESNKKFLPFQQFGRDIADTTDAVRDP
QTLEILDITPCSFGGVSVITPGTNTSNQVAVLYQGVNCTEVPVAIHADQLTPTWRVYS
TGSNVFQTRAGCLIGAEYVNNSYECDIPIGAGICASYQTQTKSHASVASQSIIAYTMSL
GAENSVAYSNNSIAIPTNFTISVTTEILPVSMTKTSVDCTMYICGDSTECSNLLLQYGS
FCTQLKRALTGIAVEQDKNTQEVFAQVKQIYKTPPIKYFGGFNFSQILPDPSKPSKRSP
IEDLLFNKVTLADAGFIKQYGDCLGDIAARDLICAQKENGLTVLPPLLTDEMIAQYTS
ALLAGTITSGWTFGAGPALQIPFPMQMAYRENGIGVTQNVLYENQKLIANQFNSAIG
KIQDSLSSTPSALGKLQDVVNHNAQALNTLVKQLSSKFGAISSVLNDILSRLDPPEAE
VQIDRLITGRLQSLQTYVTQQLIRAAEIRASANLAATKMSECVLGQSKRVDFCGKGY
HLMSFPQSAPHGVVFLHVTYVPAQEKNFTTAPAICHDGKAHFPREGVFVSNGTHWF
VTQRNFYEPQIITTDNTFVSGNCDVVIGIVNNTVYDPLQPELDSFKEELDKYFKNHTS
PDVDLGDISGINASVVNIQKEIDRLNEVAKNLNESLIDLQELGKYEQYIKWPGGGGSL
ITYIVLTIISLVFGILSLILACYLMYKQKAQQKTLLWLGNNTLDQMRATTKM*
Nucleotide Sequence of BA.5 (Signal peptide is in bold and underlined; Spike
ectodomain is underlined): SEQ ID NO: 76
ATGTTCGTGTTTCTGGTGCTGCTGCCTCTGGTGTCCAGCCAGTGTGTGAACCT
GatcACAAGAACCCAGagcTACACCAACAGCTTTACCAGAGGCGTGTACTACCCCG
ACAAGGTGTTCAGATCCAGCGTGCTGCACTCTACCCAGGACCTGTTCCTGCCTTT
CTTCAGCAACGTGACCTGGTTCCACGCCATCTCCGGCACCAATGGCACCAAGAG
ATTCGACAACCCCGTGCTGCCCTTCAACGACGGGGTGTACTTTGCCAGCACCGAG
AAGTCCAACATCATCAGAGGCTGGATCTTCGGCACCACACTGGACAGCAAGACC
CAGAGCCTGCTGATCGTGAACAACGCCACCAACGTGGTCATCAAAGTGTGCGAG
TTCCAGTTCTGCAACGACCCCTTCCTGgacGTCTACTATCACAAGAACAACAAGAG
CTGGATGGAAAGCGAGTTCCGGGTGTACAGCAGCGCCAACAACTGCACCTTCGA
GTACGTGTCCCAGCCTTTCCTGATGGACCTGGAAGGCAAGCAGGGCAACTTCAA
GAACCTGCGCGAGTTCGTGTTCAAGAACATCGACGGCTACTTCAAGATCTACAGC
AAGCACACCCCTATCAACCTCggcCGGGATCTGCCTCAGGGCTTCTCTGCTCTGGA
ACCCCTGGTGGATCTGCCCATCGGCATCAACATCACCCGGTTTCAGACACTGCTG
GCCCTGCACAGAAGCTACCTGACACCTGGCGATAGCAGCAGCGGATGGACAGCT
GGTGCCGCCGCTTACTATGTGGGCTACCTGCAGCCTAGAACCTTTCTGCTGAAGT
ACAACGAGAACGGCACCATCACCGACGCCGTGGATTGTGCTCTGGATCCTCTGA
GCGAGACAAAGTGCACCCTGAAGTCCTTCACCGTGGAgAAGGGCATCTACCAGA
CCAGCAACTTCCGGGTGCAGCCCACCGAATCCATCGTGCGGTTCCCCAATATCAC
CAATCTGTGCCCCTTCgacGAGGTGTTCAATGCCACCAGATTCGCCTCTGTGTACG
CCTGGAACCGGAAGCGGATCAGCAATTGCGTGGCCGACTACTCCGTGCTGTACA
ACttcGCCcctTTCttcgccTTCAAGTGCTACGGCGTGTCCCCTACCAAGCTGAACGACC
TGTGCTTCACAAACGTGTACGCCGACAGCTTCGTGATCCGGGGAaacGAAGTGagcC
AGATTGCCCCTGGACAGACAGGCaacATCGCCGACTACAACTACAAGCTGCCCGA
CGACTTCACCGGCTGTGTGATTGCCTGGAACAGCAACaagCTGGACTCCAAAGTCa
gcGGCAACTACAATTACcggTACCGGCTGTTCCGGAAGTCCAATCTGAAGCCCTTC
GAGCGGGACATCTCCACCGAGATCTATCAGGCCGGCaacaagCCTTGTAACGGCGT
GgccGGCgtgAACTGCTACTTCCCACTGcagTCCTACGGCTTTcggCCCACAtacGGCGT
GGGCcacCAGCCCTACAGAGTGGTGGTGCTGAGCTTCGAACTGCTGCATGCCCCTG
CCACAGTGTGCGGCCCTAAGAAgAGCACCAATCTCGTGAAGAACAAATGCGTGA
ACTTCAACTTCAACGGCCTGACCGGCACCGGCGTGCTGACAGAGAGCAACAAGA
AGTTCCTGCCATTCCAGCAGTTTGGCCGGGATATCGCCGATACCACAGACGCCGT
TAGAGATCCCCAGACACTGGAAATCCTGGACATCACCCCTTGCAGCTTCGGCGG
AGTGTCTGTGATCACCCCTGGCACCAACACCAGCAATCAGGTGGCAGTGCTGTAC
CAGggcGTGAACTGTACCGAAGTGCCCGTGGCCATTCACGCCGATCAGCTGACAC
CTACATGGCGGGTGTACTCCACCGGCAGCAATGTGTTTCAGACCAGAGCCGGCT
GTCTGATCGGAGCCGAGtacGTGAACAATAGCTACGAGTGCGACATCCCCATCGG
CGCTGGCATCTGTGCCAGCTACCAGACACAGACAaagAGCcacGCCTCTGTGGCCA
GCCAGAGCATCATTGCCTACACAATGTCTCTGGGCGCCGAGAACAGCGTGGCCT
ACTCCAACAACTCTATCGCTATCCCCACCAACTTCACCATCAGCGTGACCACAGA
GATCCTGCCTGTGTCCATGACCAAGACCAGCGTGGACTGCACCATGTACATCTGC
GGCGATTCCACCGAGTGCTCCAACCTGCTGCTGCAGTACGGCAGCTTCTGCACCC
AGCTGaagAGAGCCCTGACAGGGATCGCCGTGGAACAGGACAAGAACACCCAAG
AGGTGTTCGCCCAAGTGAAGCAGATCTACAAGACCCCTCCTATCAAGtacTTCGGC
GGCTTCAATTTCAGCCAGATTCTGCCCGATCCTAGCAAGCCCAGCAAGCGGAGCc
ctATCGAGGACCTGCTGTTCAACAAAGTGACACTGGCCGACGCCGGCTTCATCAA
GCAGTATGGCGATTGTCTGGGCGACATTGCCGCCAGGGATCTGATTTGCGCCCAG
AAGTTTAACGGACTGACAGTGCTGCCTCCTCTGCTGACCGATGAGATGATCGCCC
AGTACACATCTGCCCTGCTGGCCGGCACAATCACAAGCGGCTGGACATTTGGAG
CTGGCcctGCTCTGCAGATCCCCTTTccaATGCAGATGGCCTACCGGTTCAACGGCA
TCGGAGTGACCCAGAATGTGCTGTACGAGAACCAGAAGCTGATCGCCAACCAGT
TCAACAGCGCCATCGGCAAGATCCAGGACAGCCTGAGCAGCACAcccAGCGCCCT
GGGAAAGCTGCAGGACGTGGTCAACcacAATGCCCAGGCACTGAACACCCTGGTC
AAGCAGCTGTCCTCCaagTTCGGCGCCATCAGCTCTGTGCTGAACGATATCCTGAG
CAGACTGGACccccctGAAGCCGAGGTGCAGATCGACAGACTGATCACCGGAAGGC
TGCAGTCCCTGCAGACCTACGTTACCCAGCAGCTGATCAGAGCCGCCGAGATTA
GAGCCTCTGCCAATCTGGCCGCCACCAAGATGTCTGAGTGTGTGCTGGGCCAGA
GCAAGAGAGTGGACTTTTGCGGCAAGGGCTACCACCTGATGAGCTTCCCTCAGTC
TGCCCCTCACGGCGTGGTGTTTCTGCACGTGACATACGTGCCCGCTCAAGAGAAG
AATTTCACCACCGCTCCAGCCATCTGCCACGACGGCAAAGCCCACTTTCCTAGAG
AAGGCGTGTTCGTGTCCAACGGCACCCATTGGTTCGTGACCCAGCGGAACTTCTA
CGAGCCCCAGATCATCACCACCGACAACACCTTCGTGTCTGGCAACTGCGACGTC
GTGATCGGCATTGTGAACAATACCGTGTACGACCCTCTGCAGCCCGAGCTGGAC
AGCTTCAAAGAGGAACTGGATAAGTACTTTAAGAACCACACAAGCCCCGACGTG
GACCTGGGCGATATCAGCGGAATCAATGCCAGCGTCGTGAACATCCAGAAAGAG
ATCGACCGGCTGAACGAGGTGGCCAAGAATCTGAACGAGAGCCTGATCGACCTG
CAAGAACTGGGGAAGTACGAGCAGTACATCAAGTGGCCC
Amino Acid Sequence of BA.5 (Signal peptide is in bold and underlined; Spike
ectodomain is underlined): SEQ ID NO: 77
MFVFLVLLPLVSSQCVNLITRTQSYTNSFTRGVYYPDKVFRSSVLHSTQDLFLPFFSN
VTWFHAISGTNGTKRFDNPVLPFNDGVYFASTEKSNIIRGWIFGTTLDSKTQSLLIVN
NATNVVIKVCEFQFCNDPFLDVYYHKNNKSWMESEFRVYSSANNCTFEYVSQPFLM
DLEGKQGNFKNLREFVFKNIDGYFKIYSKHTPINLGRDLPQGFSALEPLVDLPIGINIT
RFQTLLALHRSYLTPGDSSSGWTAGAAAYYVGYLQPRTFLLKYNENGTITDAVDCA
LDPLSETKCTLKSFTVEKGIYQTSNFRVQPTESIVRFPNITNLCPFDEVFNATRFASVY
AWNRKRISNCVADYSVLYNFAPFFAFKCYGVSPTKLNDLCFTNVYADSFVIRGNEVS
QIAPGQTGNIADYNYKLPDDFTGCVIAWNSNKLDSKVSGNYNYRYRLFRKSNLKPFE
RDISTEIYQAGNKPCNGVAGVNCYFPLQSYGFRPTYGVGHQPYRVVVLSFELLHAPA
TVCGPKKSTNLVKNKCVNFNFNGLTGTGVLTESNKKFLPFQQFGRDIADTTDAVRDP
QTLEILDITPCSFGGVSVITPGTNTSNQVAVLYQGVNCTEVPVAIHADQLTPTWRVYS
TGSNVFQTRAGCLIGAEYVNNSYECDIPIGAGICASYQTQTKSHASVASQSIIAYTMSL
GAENSVAYSNNSIAIPTNFTISVTTEILPVSMTKTSVDCTMYICGDSTECSNLLLQYGS
FCTQLKRALTGIAVEQDKNTQEVFAQVKQIYKTPPIKYFGGFNFSQILPDPSKPSKRSP
IEDLLFNKVTLADAGFIKQYGDCLGDIAARDLICAQKFNGLTVLPPLLTDEMIAQYTS
ALLAGTITSGWTFGAGPALQIPFPMQMAYRENGIGVTQNVLYENQKLIANQFNSAIG
KIQDSLSSTPSALGKLQDVVNHNAQALNTLVKQLSSKFGAISSVLNDILSRLDPPEAE
VQIDRLITGRLQSLQTYVTQQLIRAAEIRASANLAATKMSECVLGQSKRVDFCGKGY
HLMSFPQSAPHGVVFLHVTYVPAQEKNFTTAPAICHDGKAHFPREGVFVSNGTHWF
VTQRNFYEPQIITTDNTFVSGNCDVVIGIVNNTVYDPLQPELDSFKEELDKYFKNHTS
PDVDLGDISGINASVVNIQKEIDRLNEVAKNLNESLIDLQELGKYEQYIKWP
Nucleotide Sequence of BA.5 (Spike ectodomain without signal peptide is
underlined): SEQ ID NO: 78
CAGTGTGTGAACCTGatcACAAGAACCCAGagcTACACCAACAGCTTTACCAGAGG
CGTGTACTACCCCGACAAGGTGTTCAGATCCAGCGTGCTGCACTCTACCCAGGAC
CTGTTCCTGCCTTTCTTCAGCAACGTGACCTGGTTCCACGCCATCTCCGGCACCAA
TGGCACCAAGAGATTCGACAACCCCGTGCTGCCCTTCAACGACGGGGTGTACTTT
GCCAGCACCGAGAAGTCCAACATCATCAGAGGCTGGATCTTCGGCACCACACTG
GACAGCAAGACCCAGAGCCTGCTGATCGTGAACAACGCCACCAACGTGGTCATC
AAAGTGTGCGAGTTCCAGTTCTGCAACGACCCCTTCCTGgacGTCTACTATCACAA
GAACAACAAGAGCTGGATGGAAAGCGAGTTCCGGGTGTACAGCAGCGCCAACA
ACTGCACCTTCGAGTACGTGTCCCAGCCTTTCCTGATGGACCTGGAAGGCAAGCA
GGGCAACTTCAAGAACCTGCGCGAGTTCGTGTTCAAGAACATCGACGGCTACTTC
AAGATCTACAGCAAGCACACCCCTATCAACCTCggcCGGGATCTGCCTCAGGGCTT
CTCTGCTCTGGAACCCCTGGTGGATCTGCCCATCGGCATCAACATCACCCGGTTT
CAGACACTGCTGGCCCTGCACAGAAGCTACCTGACACCTGGCGATAGCAGCAGC
GGATGGACAGCTGGTGCCGCCGCTTACTATGTGGGCTACCTGCAGCCTAGAACCT
TTCTGCTGAAGTACAACGAGAACGGCACCATCACCGACGCCGTGGATTGTGCTCT
GGATCCTCTGAGCGAGACAAAGTGCACCCTGAAGTCCTTCACCGTGGAgAAGGG
CATCTACCAGACCAGCAACTTCCGGGTGCAGCCCACCGAATCCATCGTGCGGTTC
CCCAATATCACCAATCTGTGCCCCTTCgacGAGGTGTTCAATGCCACCAGATTCGC
CTCTGTGTACGCCTGGAACCGGAAGCGGATCAGCAATTGCGTGGCCGACTACTCC
GTGCTGTACAACttcGCCcctTTCttcgccTTCAAGTGCTACGGCGTGTCCCCTACCAAG
CTGAACGACCTGTGCTTCACAAACGTGTACGCCGACAGCTTCGTGATCCGGGGAa
acGAAGTGagcCAGATTGCCCCTGGACAGACAGGCaacATCGCCGACTACAACTACA
AGCTGCCCGACGACTTCACCGGCTGTGTGATTGCCTGGAACAGCAACaagCTGGA
CTCCAAAGTCagcGGCAACTACAATTACcggTACCGGCTGTTCCGGAAGTCCAATCT
GAAGCCCTTCGAGCGGGACATCTCCACCGAGATCTATCAGGCCGGCaacaagCCTTG
TAACGGCGTGgccGGCgtgAACTGCTACTTCCCACTGcagTCCTACGGCTTTcggCCCA
CAtacGGCGTGGGCcacCAGCCCTACAGAGTGGTGGTGCTGAGCTTCGAACTGCTGC
ATGCCCCTGCCACAGTGTGCGGCCCTAAGAAgAGCACCAATCTCGTGAAGAACA
AATGCGTGAACTTCAACTTCAACGGCCTGACCGGCACCGGCGTGCTGACAGAGA
GCAACAAGAAGTTCCTGCCATTCCAGCAGTTTGGCCGGGATATCGCCGATACCAC
AGACGCCGTTAGAGATCCCCAGACACTGGAAATCCTGGACATCACCCCTTGCAG
CTTCGGCGGAGTGTCTGTGATCACCCCTGGCACCAACACCAGCAATCAGGTGGC
AGTGCTGTACCAGggcGTGAACTGTACCGAAGTGCCCGTGGCCATTCACGCCGAT
CAGCTGACACCTACATGGCGGGTGTACTCCACCGGCAGCAATGTGTTTCAGACCA
GAGCCGGCTGTCTGATCGGAGCCGAGtacGTGAACAATAGCTACGAGTGCGACAT
CCCCATCGGCGCTGGCATCTGTGCCAGCTACCAGACACAGACAaagAGCcacGCCT
CTGTGGCCAGCCAGAGCATCATTGCCTACACAATGTCTCTGGGCGCCGAGAACA
GCGTGGCCTACTCCAACAACTCTATCGCTATCCCCACCAACTTCACCATCAGCGT
GACCACAGAGATCCTGCCTGTGTCCATGACCAAGACCAGCGTGGACTGCACCAT
GTACATCTGCGGCGATTCCACCGAGTGCTCCAACCTGCTGCTGCAGTACGGCAGC
TTCTGCACCCAGCTGaagAGAGCCCTGACAGGGATCGCCGTGGAACAGGACAAGA
ACACCCAAGAGGTGTTCGCCCAAGTGAAGCAGATCTACAAGACCCCTCCTATCA
AGtacTTCGGCGGCTTCAATTTCAGCCAGATTCTGCCCGATCCTAGCAAGCCCAGC
AAGCGGAGCcctATCGAGGACCTGCTGTTCAACAAAGTGACACTGGCCGACGCCG
GCTTCATCAAGCAGTATGGCGATTGTCTGGGCGACATTGCCGCCAGGGATCTGAT
TTGCGCCCAGAAGTTTAACGGACTGACAGTGCTGCCTCCTCTGCTGACCGATGAG
ATGATCGCCCAGTACACATCTGCCCTGCTGGCCGGCACAATCACAAGCGGCTGG
ACATTTGGAGCTGGCcctGCTCTGCAGATCCCCTTTccaATGCAGATGGCCTACCGG
TTCAACGGCATCGGAGTGACCCAGAATGTGCTGTACGAGAACCAGAAGCTGATC
GCCAACCAGTTCAACAGCGCCATCGGCAAGATCCAGGACAGCCTGAGCAGCACA
cccAGCGCCCTGGGAAAGCTGCAGGACGTGGTCAACcacAATGCCCAGGCACTGAA
CACCCTGGTCAAGCAGCTGTCCTCCaagTTCGGCGCCATCAGCTCTGTGCTGAACG
ATATCCTGAGCAGACTGGACccccctGAAGCCGAGGTGCAGATCGACAGACTGATC
ACCGGAAGGCTGCAGTCCCTGCAGACCTACGTTACCCAGCAGCTGATCAGAGCC
GCCGAGATTAGAGCCTCTGCCAATCTGGCCGCCACCAAGATGTCTGAGTGTGTGC
TGGGCCAGAGCAAGAGAGTGGACTTTTGCGGCAAGGGCTACCACCTGATGAGCT
TCCCTCAGTCTGCCCCTCACGGCGTGGTGTTTCTGCACGTGACATACGTGCCCGC
TCAAGAGAAGAATTTCACCACCGCTCCAGCCATCTGCCACGACGGCAAAGCCCA
CTTTCCTAGAGAAGGCGTGTTCGTGTCCAACGGCACCCATTGGTTCGTGACCCAG
CGGAACTTCTACGAGCCCCAGATCATCACCACCGACAACACCTTCGTGTCTGGCA
ACTGCGACGTCGTGATCGGCATTGTGAACAATACCGTGTACGACCCTCTGCAGCC
CGAGCTGGACAGCTTCAAAGAGGAACTGGATAAGTACTTTAAGAACCACACAAG
CCCCGACGTGGACCTGGGCGATATCAGCGGAATCAATGCCAGCGTCGTGAACAT
CCAGAAAGAGATCGACCGGCTGAACGAGGTGGCCAAGAATCTGAACGAGAGCC
TGATCGACCTGCAAGAACTGGGGAAGTACGAGCAGTACATCAAGTGGCCC
Amino Acid Sequence of BA.5 (Spike ectodomain without signal peptide is underlined):
SEQ ID NO: 79
QCVNLITRTQSYTNSFTRGVYYPDKVFRSSVLHSTQDLFLPFFSNVTWFHAISGTNGT
KRFDNPVLPFNDGVYFASTEKSNIIRGWIFGTTLDSKTQSLLIVNNATNVVIKVCEFQF
CNDPFLDVYYHKNNKSWMESEFRVYSSANNCTFEYVSQPFLMDLEGKQGNFKNLR
EFVFKNIDGYFKIYSKHTPINLGRDLPQGFSALEPLVDLPIGINITRFQTLLALHRSYLT
PGDSSSGWTAGAAAYYVGYLQPRTFLLKYNENGTITDAVDCALDPLSETKCTLKSFT
VEKGIYQTSNERVQPTESIVRFPNITNLCPFDEVFNATRFASVYAWNRKRISNCVADY
SVLYNFAPFFAFKCYGVSPTKLNDLCFTNVYADSFVIRGNEVSQIAPGQTGNIADYN
YKLPDDFTGCVIAWNSNKLDSKVSGNYNYRYRLFRKSNLKPFERDISTEIYQAGNKP
CNGVAGVNCYFPLQSYGFRPTYGVGHQPYRVVVLSFELLHAPATVCGPKKSTNLVK
NKCVNFNFNGLTGTGVLTESNKKFLPFQQFGRDIADTTDAVRDPQTLEILDITPCSFG
GVSVITPGTNTSNQVAVLYQGVNCTEVPVAIHADQLTPTWRVYSTGSNVFQTRAGC
LIGAEYVNNSYECDIPIGAGICASYQTQTKSHASVASQSIIAYTMSLGAENSVAYSNN
SIAIPTNFTISVTTEILPVSMTKTSVDCTMYICGDSTECSNLLLQYGSFCTQLKRALTGI
AVEQDKNTQEVFAQVKQIYKTPPIKYFGGFNFSQILPDPSKPSKRSPIEDLLFNKVTLA
DAGFIKQYGDCLGDIAARDLICAQKENGLTVLPPLLTDEMIAQYTSALLAGTITSGWT
FGAGPALQIPFPMQMAYRENGIGVTQNVLYENQKLIANQFNSAIGKIQDSLSSTPSAL
GKLQDVVNHNAQALNTLVKQLSSKFGAISSVLNDILSRLDPPEAEVQIDRLITGRLQS
LQTYVTQQLIRAAEIRASANLAATKMSECVLGQSKRVDFCGKGYHLMSFPQSAPHG
VVFLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISGINA
SVVNIQKEIDRLNEVAKNLNESLIDLQELGKYEQYIKWP
Nucleotide Sequence of BA.5 S371 S373 S375 L452 (BA.5 SSS L452) (Signal peptide is
in bold and underlined; GS linker is in italics; TM and CT From F is in bold and
double underlined; Spike ectodomain is underlined; S371 S373 S375 is in bold, italics
and underlined): SEQ ID NO: 80
ATGTTCGTGTTTCTGGTGCTGCTGCCTCTGGTGTCCAGCCAGTGTGTGAACCT
GatcACAAGAACCCAGagcTACACCAACAGCTTTACCAGAGGCGTGTACTACCCCG
ACAAGGTGTTCAGATCCAGCGTGCTGCACTCTACCCAGGACCTGTTCCTGCCTTT
CTTCAGCAACGTGACCTGGTTCCACGCCATCTCCGGCACCAATGGCACCAAGAG
ATTCGACAACCCCGTGCTGCCCTTCAACGACGGGGTGTACTTTGCCAGCACCGAG
AAGTCCAACATCATCAGAGGCTGGATCTTCGGCACCACACTGGACAGCAAGACC
CAGAGCCTGCTGATCGTGAACAACGCCACCAACGTGGTCATCAAAGTGTGCGAG
TTCCAGTTCTGCAACGACCCCTTCCTGgacGTCTACTATCACAAGAACAACAAGAG
CTGGATGGAAAGCGAGTTCCGGGTGTACAGCAGCGCCAACAACTGCACCTTCGA
GTACGTGTCCCAGCCTTTCCTGATGGACCTGGAAGGCAAGCAGGGCAACTTCAA
GAACCTGCGCGAGTTCGTGTTCAAGAACATCGACGGCTACTTCAAGATCTACAGC
AAGCACACCCCTATCAACCTCggcCGGGATCTGCCTCAGGGCTTCTCTGCTCTGGA
ACCCCTGGTGGATCTGCCCATCGGCATCAACATCACCCGGTTTCAGACACTGCTG
GCCCTGCACAGAAGCTACCTGACACCTGGCGATAGCAGCAGCGGATGGACAGCT
GGTGCCGCCGCTTACTATGTGGGCTACCTGCAGCCTAGAACCTTTCTGCTGAAGT
ACAACGAGAACGGCACCATCACCGACGCCGTGGATTGTGCTCTGGATCCTCTGA
GCGAGACAAAGTGCACCCTGAAGTCCTTCACCGTGGAgAAGGGCATCTACCAGA
CCAGCAACTTCCGGGTGCAGCCCACCGAATCCATCGTGCGGTTCCCCAATATCAC
CAATCTGTGCCCCTTCgacGAGGTGTTCAATGCCACCAGATTCGCCTCTGTGTACG
CCTGGAACCGGAAGCGGATCAGCAATTGCGTGGCCGACTACTCCGTGCTGTACA
ACtccGCCagcTTCagcgccTTCAAGTGCTACGGCGTGTCCCCTACCAAGCTGAACGAC
CTGTGCTTCACAAACGTGTACGCCGACAGCTTCGTGATCCGGGGAaacGAAGTGagc
CAGATTGCCCCTGGACAGACAGGCaacATCGCCGACTACAACTACAAGCTGCCCG
ACGACTTCACCGGCTGTGTGATTGCCTGGAACAGCAACaagCTGGACTCCAAAGTC
agcGGCAACTACAATTACctgTACCGGCTGTTCCGGAAGTCCAATCTGAAGCCCTTC
GAGCGGGACATCTCCACCGAGATCTATCAGGCCGGCaacaagCCTTGTAACGGCGT
GgccGGCgtgAACTGCTACTTCCCACTGcagTCCTACGGCTTTcggCCCACAtacGGCGT
GGGCcacCAGCCCTACAGAGTGGTGGTGCTGAGCTTCGAACTGCTGCATGCCCCTG
CCACAGTGTGCGGCCCTAAGAAgAGCACCAATCTCGTGAAGAACAAATGCGTGA
ACTTCAACTTCAACGGCCTGACCGGCACCGGCGTGCTGACAGAGAGCAACAAGA
AGTTCCTGCCATTCCAGCAGTTTGGCCGGGATATCGCCGATACCACAGACGCCGT
TAGAGATCCCCAGACACTGGAAATCCTGGACATCACCCCTTGCAGCTTCGGCGG
AGTGTCTGTGATCACCCCTGGCACCAACACCAGCAATCAGGTGGCAGTGCTGTAC
CAGggcGTGAACTGTACCGAAGTGCCCGTGGCCATTCACGCCGATCAGCTGACAC
CTACATGGCGGGTGTACTCCACCGGCAGCAATGTGTTTCAGACCAGAGCCGGCT
GTCTGATCGGAGCCGAGtacGTGAACAATAGCTACGAGTGCGACATCCCCATCGG
CGCTGGCATCTGTGCCAGCTACCAGACACAGACAaagAGCcacGCCTCTGTGGCCA
GCCAGAGCATCATTGCCTACACAATGTCTCTGGGCGCCGAGAACAGCGTGGCCT
ACTCCAACAACTCTATCGCTATCCCCACCAACTTCACCATCAGCGTGACCACAGA
GATCCTGCCTGTGTCCATGACCAAGACCAGCGTGGACTGCACCATGTACATCTGC
GGCGATTCCACCGAGTGCTCCAACCTGCTGCTGCAGTACGGCAGCTTCTGCACCC
AGCTGaagAGAGCCCTGACAGGGATCGCCGTGGAACAGGACAAGAACACCCAAG
AGGTGTTCGCCCAAGTGAAGCAGATCTACAAGACCCCTCCTATCAAGtacTTCGGC
GGCTTCAATTTCAGCCAGATTCTGCCCGATCCTAGCAAGCCCAGCAAGCGGAGCc
ctATCGAGGACCTGCTGTTCAACAAAGTGACACTGGCCGACGCCGGCTTCATCAA
GCAGTATGGCGATTGTCTGGGCGACATTGCCGCCAGGGATCTGATTTGCGCCCAG
AAGTTTAACGGACTGACAGTGCTGCCTCCTCTGCTGACCGATGAGATGATCGCCC
AGTACACATCTGCCCTGCTGGCCGGCACAATCACAAGCGGCTGGACATTTGGAG
CTGGCcctGCTCTGCAGATCCCCTTTccaATGCAGATGGCCTACCGGTTCAACGGCA
TCGGAGTGACCCAGAATGTGCTGTACGAGAACCAGAAGCTGATCGCCAACCAGT
TCAACAGCGCCATCGGCAAGATCCAGGACAGCCTGAGCAGCACAcccAGCGCCCT
GGGAAAGCTGCAGGACGTGGTCAACcacAATGCCCAGGCACTGAACACCCTGGTC
AAGCAGCTGTCCTCCaagTTCGGCGCCATCAGCTCTGTGCTGAACGATATCCTGAG
CAGACTGGACccccctGAAGCCGAGGTGCAGATCGACAGACTGATCACCGGAAGGC
TGCAGTCCCTGCAGACCTACGTTACCCAGCAGCTGATCAGAGCCGCCGAGATTA
GAGCCTCTGCCAATCTGGCCGCCACCAAGATGTCTGAGTGTGTGCTGGGCCAGA
GCAAGAGAGTGGACTTTTGCGGCAAGGGCTACCACCTGATGAGCTTCCCTCAGTC
TGCCCCTCACGGCGTGGTGTTTCTGCACGTGACATACGTGCCCGCTCAAGAGAAG
AATTTCACCACCGCTCCAGCCATCTGCCACGACGGCAAAGCCCACTTTCCTAGAG
AAGGCGTGTTCGTGTCCAACGGCACCCATTGGTTCGTGACCCAGCGGAACTTCTA
CGAGCCCCAGATCATCACCACCGACAACACCTTCGTGTCTGGCAACTGCGACGTC
GTGATCGGCATTGTGAACAATACCGTGTACGACCCTCTGCAGCCCGAGCTGGAC
AGCTTCAAAGAGGAACTGGATAAGTACTTTAAGAACCACACAAGCCCCGACGTG
GACCTGGGCGATATCAGCGGAATCAATGCCAGCGTCGTGAACATCCAGAAAGAG
ATCGACCGGCTGAACGAGGTGGCCAAGAATCTGAACGAGAGCCTGATCGACCTG
CAAGAACTGGGGAAGTACGAGCAGTACATCAAGTGGCCCggcggaggtgggtcgCTCAT
AACATACATCGTCCTGACTATAATCAGCTTGGTATTTGGTATTTTGTCTTTGA
TTCTTGCATGCTATTTGATGTATAAACAGAAAGCTCAGCAGAAGACTCTCCT
GTGGCTCGGTAACAACACACTCGACCAGATGAGAGCAACTACAAAGATGTG
A
Amino Acid Sequence of BA.5 S371 S373 S375 L452 (BA.5 SSS L452) (Signal peptide is
in bold and underlined; GS linker is in italics; TM and CT From F is in bold and double
underlined; Spike ectodomain is underlined; S371 S373 S375 is in bold, italics and
underlined): SEQ ID NO: 81
MFVFLVLLPLVSSQCVNLITRTQSYTNSFTRGVYYPDKVFRSSVLHSTQDLFLPFFSN
VTWFHAISGTNGTKRFDNPVLPFNDGVYFASTEKSNIIRGWIFGTTLDSKTQSLLIVN
NATNVVIKVCEFQFCNDPFLDVYYHKNNKSWMESEFRVYSSANNCTFEYVSQPFLM
DLEGKQGNFKNLREFVFKNIDGYFKIYSKHTPINLGRDLPQGFSALEPLVDLPIGINIT
RFQTLLALHRSYLTPGDSSSGWTAGAAAYYVGYLQPRTFLLKYNENGTITDAVDCA
LDPLSETKCTLKSFTVEKGIYQTSNFRVQPTESIVRFPNITNLCPFDEVFNATRFASVY
AWNRKRISNCVADYSVLYNSASFSAFKCYGVSPTKLNDLCFTNVYADSFVIRGNEVS
QIAPGQTGNIADYNYKLPDDFTGCVIAWNSNKLDSKVSGNYNYLYRLFRKSNLKPFE
RDISTEIYQAGNKPCNGVAGVNCYFPLQSYGFRPTYGVGHQPYRVVVLSFELLHAPA
TVCGPKKSTNLVKNKCVNFNFNGLTGTGVLTESNKKFLPFQQFGRDIADTTDAVRDP
QTLEILDITPCSFGGVSVITPGTNTSNQVAVLYQGVNCTEVPVAIHADQLTPTWRVYS
TGSNVFQTRAGCLIGAEYVNNSYECDIPIGAGICASYQTQTKSHASVASQSIIAYTMSL
GAENSVAYSNNSIAIPTNFTISVTTEILPVSMTKTSVDCTMYICGDSTECSNLLLQYGS
FCTQLKRALTGIAVEQDKNTQEVFAQVKQIYKTPPIKYFGGFNFSQILPDPSKPSKRSP
IEDLLFNKVTLADAGFIKQYGDCLGDIAARDLICAQKFNGLTVLPPLLTDEMIAQYTS
ALLAGTITSGWTFGAGPALQIPFPMQMAYRENGIGVTQNVLYENQKLIANQFNSAIG
KIQDSLSSTPSALGKLQDVVNHNAQALNTLVKQLSSKFGAISSVLNDILSRLDPPEAE
VQIDRLITGRLQSLQTYVTQQLIRAAEIRASANLAATKMSECVLGQSKRVDFCGKGY
HLMSFPQSAPHGVVFLHVTYVPAQEKNFTTAPAICHDGKAHFPREGVFVSNGTHWF
VTQRNFYEPQIITTDNTFVSGNCDVVIGIVNNTVYDPLQPELDSFKEELDKYFKNHTS
PDVDLGDISGINASVVNIQKEIDRLNEVAKNLNESLIDLQELGKYEQYIKWPGGGGSL
ITYIVLTIISLVFGILSLILACYLMYKQKAQQKTLLWLGNNTLDQMRATTKM*
Nucleotide Sequence of BA.5 S371 S373 S375 L452 (BA.5 SSS L452) (Signal peptide is
in bold and underlined; Spike ectodomain is underlined; S371 S373 S375 is in bold,
italics and underlined): SEQ ID NO: 82
ATGTTCGTGTTTCTGGTGCTGCTGCCTCTGGTGTCCAGCCAGTGTGTGAACCT
GatcACAAGAACCCAGagcTACACCAACAGCTTTACCAGAGGCGTGTACTACCCCG
ACAAGGTGTTCAGATCCAGCGTGCTGCACTCTACCCAGGACCTGTTCCTGCCTTT
CTTCAGCAACGTGACCTGGTTCCACGCCATCTCCGGCACCAATGGCACCAAGAG
ATTCGACAACCCCGTGCTGCCCTTCAACGACGGGGTGTACTTTGCCAGCACCGAG
AAGTCCAACATCATCAGAGGCTGGATCTTCGGCACCACACTGGACAGCAAGACC
CAGAGCCTGCTGATCGTGAACAACGCCACCAACGTGGTCATCAAAGTGTGCGAG
TTCCAGTTCTGCAACGACCCCTTCCTGgacGTCTACTATCACAAGAACAACAAGAG
CTGGATGGAAAGCGAGTTCCGGGTGTACAGCAGCGCCAACAACTGCACCTTCGA
GTACGTGTCCCAGCCTTTCCTGATGGACCTGGAAGGCAAGCAGGGCAACTTCAA
GAACCTGCGCGAGTTCGTGTTCAAGAACATCGACGGCTACTTCAAGATCTACAGC
AAGCACACCCCTATCAACCTCggcCGGGATCTGCCTCAGGGCTTCTCTGCTCTGGA
ACCCCTGGTGGATCTGCCCATCGGCATCAACATCACCCGGTTTCAGACACTGCTG
GCCCTGCACAGAAGCTACCTGACACCTGGCGATAGCAGCAGCGGATGGACAGCT
GGTGCCGCCGCTTACTATGTGGGCTACCTGCAGCCTAGAACCTTTCTGCTGAAGT
ACAACGAGAACGGCACCATCACCGACGCCGTGGATTGTGCTCTGGATCCTCTGA
GCGAGACAAAGTGCACCCTGAAGTCCTTCACCGTGGAgAAGGGCATCTACCAGA
CCAGCAACTTCCGGGTGCAGCCCACCGAATCCATCGTGCGGTTCCCCAATATCAC
CAATCTGTGCCCCTTCgacGAGGTGTTCAATGCCACCAGATTCGCCTCTGTGTACG
CCTGGAACCGGAAGCGGATCAGCAATTGCGTGGCCGACTACTCCGTGCTGTACA
ACtccGCCagcTTCagcgccTTCAAGTGCTACGGCGTGTCCCCTACCAAGCTGAACGAC
CTGTGCTTCACAAACGTGTACGCCGACAGCTTCGTGATCCGGGGAaacGAAGTGagc
CAGATTGCCCCTGGACAGACAGGCaacATCGCCGACTACAACTACAAGCTGCCCG
ACGACTTCACCGGCTGTGTGATTGCCTGGAACAGCAACaagCTGGACTCCAAAGTC
agcGGCAACTACAATTACctgTACCGGCTGTTCCGGAAGTCCAATCTGAAGCCCTTC
GAGCGGGACATCTCCACCGAGATCTATCAGGCCGGCaacaagCCTTGTAACGGCGT
GgccGGCgtgAACTGCTACTTCCCACTGcagTCCTACGGCTTTcggCCCACAtacGGCGT
GGGCcacCAGCCCTACAGAGTGGTGGTGCTGAGCTTCGAACTGCTGCATGCCCCTG
CCACAGTGTGCGGCCCTAAGAAgAGCACCAATCTCGTGAAGAACAAATGCGTGA
ACTTCAACTTCAACGGCCTGACCGGCACCGGCGTGCTGACAGAGAGCAACAAGA
AGTTCCTGCCATTCCAGCAGTTTGGCCGGGATATCGCCGATACCACAGACGCCGT
TAGAGATCCCCAGACACTGGAAATCCTGGACATCACCCCTTGCAGCTTCGGCGG
AGTGTCTGTGATCACCCCTGGCACCAACACCAGCAATCAGGTGGCAGTGCTGTAC
CAGggcGTGAACTGTACCGAAGTGCCCGTGGCCATTCACGCCGATCAGCTGACAC
CTACATGGCGGGTGTACTCCACCGGCAGCAATGTGTTTCAGACCAGAGCCGGCT
GTCTGATCGGAGCCGAGtacGTGAACAATAGCTACGAGTGCGACATCCCCATCGG
CGCTGGCATCTGTGCCAGCTACCAGACACAGACAaagAGCcacGCCTCTGTGGCCA
GCCAGAGCATCATTGCCTACACAATGTCTCTGGGCGCCGAGAACAGCGTGGCCT
ACTCCAACAACTCTATCGCTATCCCCACCAACTTCACCATCAGCGTGACCACAGA
GATCCTGCCTGTGTCCATGACCAAGACCAGCGTGGACTGCACCATGTACATCTGC
GGCGATTCCACCGAGTGCTCCAACCTGCTGCTGCAGTACGGCAGCTTCTGCACCC
AGCTGaagAGAGCCCTGACAGGGATCGCCGTGGAACAGGACAAGAACACCCAAG
AGGTGTTCGCCCAAGTGAAGCAGATCTACAAGACCCCTCCTATCAAGtacTTCGGC
GGCTTCAATTTCAGCCAGATTCTGCCCGATCCTAGCAAGCCCAGCAAGCGGAGCc
ctATCGAGGACCTGCTGTTCAACAAAGTGACACTGGCCGACGCCGGCTTCATCAA
GCAGTATGGCGATTGTCTGGGCGACATTGCCGCCAGGGATCTGATTTGCGCCCAG
AAGTTTAACGGACTGACAGTGCTGCCTCCTCTGCTGACCGATGAGATGATCGCCC
AGTACACATCTGCCCTGCTGGCCGGCACAATCACAAGCGGCTGGACATTTGGAG
CTGGCcctGCTCTGCAGATCCCCTTTccaATGCAGATGGCCTACCGGTTCAACGGCA
TCGGAGTGACCCAGAATGTGCTGTACGAGAACCAGAAGCTGATCGCCAACCAGT
TCAACAGCGCCATCGGCAAGATCCAGGACAGCCTGAGCAGCACAcccAGCGCCCT
GGGAAAGCTGCAGGACGTGGTCAACcacAATGCCCAGGCACTGAACACCCTGGTC
AAGCAGCTGTCCTCCaagTTCGGCGCCATCAGCTCTGTGCTGAACGATATCCTGAG
CAGACTGGACccccctGAAGCCGAGGTGCAGATCGACAGACTGATCACCGGAAGGC
TGCAGTCCCTGCAGACCTACGTTACCCAGCAGCTGATCAGAGCCGCCGAGATTA
GAGCCTCTGCCAATCTGGCCGCCACCAAGATGTCTGAGTGTGTGCTGGGCCAGA
GCAAGAGAGTGGACTTTTGCGGCAAGGGCTACCACCTGATGAGCTTCCCTCAGTC
TGCCCCTCACGGCGTGGTGTTTCTGCACGTGACATACGTGCCCGCTCAAGAGAAG
AATTTCACCACCGCTCCAGCCATCTGCCACGACGGCAAAGCCCACTTTCCTAGAG
AAGGCGTGTTCGTGTCCAACGGCACCCATTGGTTCGTGACCCAGCGGAACTTCTA
CGAGCCCCAGATCATCACCACCGACAACACCTTCGTGTCTGGCAACTGCGACGTC
GTGATCGGCATTGTGAACAATACCGTGTACGACCCTCTGCAGCCCGAGCTGGAC
AGCTTCAAAGAGGAACTGGATAAGTACTTTAAGAACCACACAAGCCCCGACGTG
GACCTGGGCGATATCAGCGGAATCAATGCCAGCGTCGTGAACATCCAGAAAGAG
ATCGACCGGCTGAACGAGGTGGCCAAGAATCTGAACGAGAGCCTGATCGACCTG
CAAGAACTGGGGAAGTACGAGCAGTACATCAAGTGGCCC
Amino Acid Sequence of BA.5 S371 S373 S375 L452 (BA.5 SSS L452) (Signal peptide is
in bold and underlined; Spike ectodomain is underlined; S371 S373 S375 is in bold,
italics and underlined): SEQ ID NO: 83
MFVFLVLLPLVSSQCVNLITRTQSYTNSFTRGVYYPDKVFRSSVLHSTQDLFLPFFSN
VTWFHAISGTNGTKRFDNPVLPFNDGVYFASTEKSNIIRGWIFGTTLDSKTQSLLIVN
NATNVVIKVCEFQFCNDPFLDVYYHKNNKSWMESEFRVYSSANNCTFEYVSQPFLM
DLEGKQGNFKNLREFVFKNIDGYFKIYSKHTPINLGRDLPQGFSALEPLVDLPIGINIT
RFQTLLALHRSYLTPGDSSSGWTAGAAAYYVGYLQPRTFLLKYNENGTITDAVDCA
LDPLSETKCTLKSFTVEKGIYQTSNFRVQPTESIVRFPNITNLCPFDEVFNATRFASVY
AWNRKRISNCVADYSVLYNSASFSAFKCYGVSPTKLNDLCFTNVYADSFVIRGNEVS
QIAPGQTGNIADYNYKLPDDFTGCVIAWNSNKLDSKVSGNYNYLYRLFRKSNLKPFE
RDISTEIYQAGNKPCNGVAGVNCYFPLQSYGFRPTYGVGHQPYRVVVLSFELLHAPA
TVCGPKKSTNLVKNKCVNFNFNGLTGTGVLTESNKKFLPFQQFGRDIADTTDAVRDP
QTLEILDITPCSFGGVSVITPGTNTSNQVAVLYQGVNCTEVPVAIHADQLTPTWRVYS
TGSNVFQTRAGCLIGAEYVNNSYECDIPIGAGICASYQTQTKSHASVASQSIIAYTMSL
GAENSVAYSNNSIAIPTNFTISVTTEILPVSMTKTSVDCTMYICGDSTECSNLLLQYGS
FCTQLKRALTGIAVEQDKNTQEVFAQVKQIYKTPPIKYFGGFNFSQILPDPSKPSKRSP
IEDLLFNKVTLADAGFIKQYGDCLGDIAARDLICAQKENGLTVLPPLLTDEMIAQYTS
ALLAGTITSGWTFGAGPALQIPFPMQMAYRENGIGVTQNVLYENQKLIANQFNSAIG
KIQDSLSSTPSALGKLQDVVNHNAQALNTLVKQLSSKFGAISSVLNDILSRLDPPEAE
VQIDRLITGRLQSLQTYVTQQLIRAAEIRASANLAATKMSECVLGQSKRVDFCGKGY
HLMSFPQSAPHGVVFLHVTYVPAQEKNFTTAPAICHDGKAHFPREGVFVSNGTHWF
VTQRNFYEPQIITTDNTFVSGNCDVVIGIVNNTVYDPLQPELDSFKEELDKYFKNHTS
PDVDLGDISGINASVVNIQKEIDRLNEVAKNLNESLIDLQELGKYEQYIKWP
Nucleotide Sequence of BA.5 S371 S373 S375 L452 (BA.5 SSS L452) (Spike ectodomain
without signal peptide is underlined; S371 S373 S375 is in bold, italics and
underlined): SEQ ID NO: 84
CAGTGTGTGAACCTGatcACAAGAACCCAGagcTACACCAACAGCTTTACCAGAGG
CGTGTACTACCCCGACAAGGTGTTCAGATCCAGCGTGCTGCACTCTACCCAGGAC
CTGTTCCTGCCTTTCTTCAGCAACGTGACCTGGTTCCACGCCATCTCCGGCACCAA
TGGCACCAAGAGATTCGACAACCCCGTGCTGCCCTTCAACGACGGGGTGTACTTT
GCCAGCACCGAGAAGTCCAACATCATCAGAGGCTGGATCTTCGGCACCACACTG
GACAGCAAGACCCAGAGCCTGCTGATCGTGAACAACGCCACCAACGTGGTCATC
AAAGTGTGCGAGTTCCAGTTCTGCAACGACCCCTTCCTGgacGTCTACTATCACAA
GAACAACAAGAGCTGGATGGAAAGCGAGTTCCGGGTGTACAGCAGCGCCAACA
ACTGCACCTTCGAGTACGTGTCCCAGCCTTTCCTGATGGACCTGGAAGGCAAGCA
GGGCAACTTCAAGAACCTGCGCGAGTTCGTGTTCAAGAACATCGACGGCTACTTC
AAGATCTACAGCAAGCACACCCCTATCAACCTCggcCGGGATCTGCCTCAGGGCTT
CTCTGCTCTGGAACCCCTGGTGGATCTGCCCATCGGCATCAACATCACCCGGTTT
CAGACACTGCTGGCCCTGCACAGAAGCTACCTGACACCTGGCGATAGCAGCAGC
GGATGGACAGCTGGTGCCGCCGCTTACTATGTGGGCTACCTGCAGCCTAGAACCT
TTCTGCTGAAGTACAACGAGAACGGCACCATCACCGACGCCGTGGATTGTGCTCT
GGATCCTCTGAGCGAGACAAAGTGCACCCTGAAGTCCTTCACCGTGGAgAAGGG
CATCTACCAGACCAGCAACTTCCGGGTGCAGCCCACCGAATCCATCGTGCGGTTC
CCCAATATCACCAATCTGTGCCCCTTCgacGAGGTGTTCAATGCCACCAGATTCGC
CTCTGTGTACGCCTGGAACCGGAAGCGGATCAGCAATTGCGTGGCCGACTACTCC
GTGCTGTACAACtccGCCagcTTCagcgccTTCAAGTGCTACGGCGTGTCCCCTACCAA
GCTGAACGACCTGTGCTTCACAAACGTGTACGCCGACAGCTTCGTGATCCGGGG
AaacGAAGTGagcCAGATTGCCCCTGGACAGACAGGCaacATCGCCGACTACAACTA
CAAGCTGCCCGACGACTTCACCGGCTGTGTGATTGCCTGGAACAGCAACaagCTG
GACTCCAAAGTCagcGGCAACTACAATTACctgTACCGGCTGTTCCGGAAGTCCAAT
CTGAAGCCCTTCGAGCGGGACATCTCCACCGAGATCTATCAGGCCGGCaacaagCCT
TGTAACGGCGTGgccGGCgtgAACTGCTACTTCCCACTGcagTCCTACGGCTTTcggCC
CACAtacGGCGTGGGCcacCAGCCCTACAGAGTGGTGGTGCTGAGCTTCGAACTGCT
GCATGCCCCTGCCACAGTGTGCGGCCCTAAGAAgAGCACCAATCTCGTGAAGAA
CAAATGCGTGAACTTCAACTTCAACGGCCTGACCGGCACCGGCGTGCTGACAGA
GAGCAACAAGAAGTTCCTGCCATTCCAGCAGTTTGGCCGGGATATCGCCGATAC
CACAGACGCCGTTAGAGATCCCCAGACACTGGAAATCCTGGACATCACCCCTTG
CAGCTTCGGCGGAGTGTCTGTGATCACCCCTGGCACCAACACCAGCAATCAGGT
GGCAGTGCTGTACCAGggcGTGAACTGTACCGAAGTGCCCGTGGCCATTCACGCC
GATCAGCTGACACCTACATGGCGGGTGTACTCCACCGGCAGCAATGTGTTTCAGA
CCAGAGCCGGCTGTCTGATCGGAGCCGAGtacGTGAACAATAGCTACGAGTGCGA
CATCCCCATCGGCGCTGGCATCTGTGCCAGCTACCAGACACAGACAaagAGCcacG
CCTCTGTGGCCAGCCAGAGCATCATTGCCTACACAATGTCTCTGGGCGCCGAGAA
CAGCGTGGCCTACTCCAACAACTCTATCGCTATCCCCACCAACTTCACCATCAGC
GTGACCACAGAGATCCTGCCTGTGTCCATGACCAAGACCAGCGTGGACTGCACC
ATGTACATCTGCGGCGATTCCACCGAGTGCTCCAACCTGCTGCTGCAGTACGGCA
GCTTCTGCACCCAGCTGaagAGAGCCCTGACAGGGATCGCCGTGGAACAGGACAA
GAACACCCAAGAGGTGTTCGCCCAAGTGAAGCAGATCTACAAGACCCCTCCTAT
CAAGtacTTCGGCGGCTTCAATTTCAGCCAGATTCTGCCCGATCCTAGCAAGCCCA
GCAAGCGGAGCcctATCGAGGACCTGTGTTCAACAAAGTGACACTGGCCGACGC
CGGCTTCATCAAGCAGTATGGCGATTGTCTGGGCGACATTGCCGCCAGGGATCTG
ATTTGCGCCCAGAAGTTTAACGGACTGACAGTGCTGCCTCCTCTGCTGACCGATG
AGATGATCGCCCAGTACACATCTGCCCTGCTGGCCGGCACAATCACAAGCGGCT
GGACATTTGGAGCTGGCcctGCTCTGCAGATCCCCTTTccaATGCAGATGGCCTACC
GGTTCAACGGCATCGGAGTGACCCAGAATGTGCTGTACGAGAACCAGAAGCTGA
TCGCCAACCAGTTCAACAGCGCCATCGGCAAGATCCAGGACAGCCTGAGCAGCA
CAcccAGCGCCCTGGGAAAGCTGCAGGACGTGGTCAACcacAATGCCCAGGCACTG
AACACCCTGGTCAAGCAGCTGTCCTCCaagTTCGGCGCCATCAGCTCTGTGCTGAA
CGATATCCTGAGCAGACTGGACccccctGAAGCCGAGGTGCAGATCGACAGACTGA
TCACCGGAAGGCTGCAGTCCCTGCAGACCTACGTTACCCAGCAGCTGATCAGAG
CCGCCGAGATTAGAGCCTCTGCCAATCTGGCCGCCACCAAGATGTCTGAGTGTGT
GCTGGGCCAGAGCAAGAGAGTGGACTTTTGCGGCAAGGGCTACCACCTGATGAG
CTTCCCTCAGTCTGCCCCTCACGGCGTGGTGTTTCTGCACGTGACATACGTGCCC
GCTCAAGAGAAGAATTTCACCACCGCTCCAGCCATCTGCCACGACGGCAAAGCC
CACTTTCCTAGAGAAGGCGTGTTCGTGTCCAACGGCACCCATTGGTTCGTGACCC
AGCGGAACTTCTACGAGCCCCAGATCATCACCACCGACAACACCTTCGTGTCTGG
CAACTGCGACGTCGTGATCGGCATTGTGAACAATACCGTGTACGACCCTCTGCAG
CCCGAGCTGGACAGCTTCAAAGAGGAACTGGATAAGTACTTTAAGAACCACACA
AGCCCCGACGTGGACCTGGGCGATATCAGCGGAATCAATGCCAGCGTCGTGAAC
ATCCAGAAAGAGATCGACCGGCTGAACGAGGTGGCCAAGAATCTGAACGAGAG
CCTGATCGACCTGCAAGAACTGGGGAAGTACGAGCAGTACATCAAGTGGCCC
Amino Acid Sequence of BA.5 S371 S373 S375 L452 (BA.5 SSS L452) (Spike
ectodomain without signal peptide is underlined; S371 S373 S375 is in bold, italics
and underlined): SEQ ID NO: 85
QCVNLITRTQSYTNSFTRGVYYPDKVFRSSVLHSTQDLFLPFFSNVTWFHAISGTNGT
KRFDNPVLPFNDGVYFASTEKSNIIRGWIFGTTLDSKTQSLLIVNNATNVVIKVCEFQF
CNDPFLDVYYHKNNKSWMESEFRVYSSANNCTFEYVSQPFLMDLEGKQGNFKNLR
EFVFKNIDGYFKIYSKHTPINLGRDLPQGFSALEPLVDLPIGINITRFQTLLALHRSYLT
PGDSSSGWTAGAAAYYVGYLQPRTFLLKYNENGTITDAVDCALDPLSETKCTLKSFT
VEKGIYQTSNERVQPTESIVRFPNITNLCPFDEVFNATRFASVYAWNRKRISNCVADY
SVLYNSASFSAFKCYGVSPTKLNDLCFTNVYADSFVIRGNEVSQIAPGQTGNIADYN
YKLPDDFTGCVIAWNSNKLDSKVSGNYNYLYRLFRKSNLKPFERDISTEIYQAGNKP
CNGVAGVNCYFPLQSYGFRPTYGVGHQPYRVVVLSFELLHAPATVCGPKKSTNLVK
NKCVNFNFNGLTGTGVLTESNKKFLPFQQFGRDIADTTDAVRDPQTLEILDITPCSFG
GVSVITPGTNTSNQVAVLYQGVNCTEVPVAIHADQLTPTWRVYSTGSNVFQTRAGC
LIGAEYVNNSYECDIPIGAGICASYQTQTKSHASVASQSIIAYTMSLGAENSVAYSNN
SIAIPTNFTISVTTEILPVSMTKTSVDCTMYICGDSTECSNLLLQYGSFCTQLKRALTGI
AVEQDKNTQEVFAQVKQIYKTPPIKYFGGFNFSQILPDPSKPSKRSPIEDLLFNKVTLA
DAGFIKQYGDCLGDIAARDLICAQKFNGLTVLPPLLTDEMIAQYTSALLAGTITSGWT
FGAGPALQIPFPMQMAYRENGIGVTQNVLYENQKLIANQFNSAIGKIQDSLSSTPSAL
GKLQDVVNHNAQALNTLVKQLSSKFGAISSVLNDILSRLDPPEAEVQIDRLITGRLQS
LQTYVTQQLIRAAEIRASANLAATKMSECVLGQSKRVDFCGKGYHLMSFPQSAPHG
VVFLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISGINA
SVVNIQKEIDRLNEVAKNLNESLIDLQELGKYEQYIKWP
Nucleotide Sequence of BQ.1.1 (Signal peptide is in bold and underlined; GS linker is
in italics; TM and CT From F is in bold and double underlined; Spike ectodomain is
underlined): SEQ ID NO: 86
ATGTTCGTGTTTCTGGTGCTGCTGCCTCTGGTGTCCAGCCAGTGTGTGAACCT
GatcACAAGAACCCAGagcTACACCAACAGCTTTACCAGAGGCGTGTACTACCCCG
ACAAGGTGTTCAGATCCAGCGTGCTGCACTCTACCCAGGACCTGTTCCTGCCTTT
CTTCAGCAACGTGACCTGGTTCCACGCCATCTCCGGCACCAATGGCACCAAGAG
ATTCGACAACCCCGTGCTGCCCTTCAACGACGGGGTGTACTTTGCCAGCACCGAG
AAGTCCAACATCATCAGAGGCTGGATCTTCGGCACCACACTGGACAGCAAGACC
CAGAGCCTGCTGATCGTGAACAACGCCACCAACGTGGTCATCAAAGTGTGCGAG
TTCCAGTTCTGCAACGACCCCTTCCTGgacGTCTACTATCACAAGAACAACAAGAG
CTGGATGGAAAGCGAGTTCCGGGTGTACAGCAGCGCCAACAACTGCACCTTCGA
GTACGTGTCCCAGCCTTTCCTGATGGACCTGGAAGGCAAGCAGGGCAACTTCAA
GAACCTGCGCGAGTTCGTGTTCAAGAACATCGACGGCTACTTCAAGATCTACAGC
AAGCACACCCCTATCAACCTCggcCGGGATCTGCCTCAGGGCTTCTCTGCTCTGGA
ACCCCTGGTGGATCTGCCCATCGGCATCAACATCACCCGGTTTCAGACACTGCTG
GCCCTGCACAGAAGCTACCTGACACCTGGCGATAGCAGCAGCGGATGGACAGCT
GGTGCCGCCGCTTACTATGTGGGCTACCTGCAGCCTAGAACCTTTCTGCTGAAGT
ACAACGAGAACGGCACCATCACCGACGCCGTGGATTGTGCTCTGGATCCTCTGA
GCGAGACAAAGTGCACCCTGAAGTCCTTCACCGTGGAgAAGGGCATCTACCAGA
CCAGCAACTTCCGGGTGCAGCCCACCGAATCCATCGTGCGGTTCCCCAATATCAC
CAATCTGTGCCCCTTCgacGAGGTGTTCAATGCCACCaccTTCGCCTCTGTGTACGCC
TGGAACCGGAAGCGGATCAGCAATTGCGTGGCCGACTACTCCGTGCTGTACAACt
tcGCCcctTTCttcgccTTCAAGTGCTACGGCGTGTCCCCTACCAAGCTGAACGACCTGT
GCTTCACAAACGTGTACGCCGACAGCTTCGTGATCCGGGGAaacGAAGTGagcCAG
ATTGCCCCTGGACAGACAGGCaacATCGCCGACTACAACTACAAGCTGCCCGACG
ACTTCACCGGCTGTGTGATTGCCTGGAACAGCAACaagCTGGACTCCaccGTCggcGG
CAACTACAATTACcggTACCGGCTGTTCCGGAAGTCCaagCTGAAGCCCTTCGAGCG
GGACATCTCCACCGAGATCTATCAGGCCGGCaacaagCCTTGTAACGGCGTGgccGG
CgtgAACTGCTACTTCCCACTGcagTCCTACGGCTTTcggCCCACAtacGGCGTGGGCca
cCAGCCCTACAGAGTGGTGGTGCTGAGCTTCGAACTGCTGCATGCCCCTGCCACA
GTGTGCGGCCCTAAGAAgAGCACCAATCTCGTGAAGAACAAATGCGTGAACTTC
AACTTCAACGGCCTGACCGGCACCGGCGTGCTGACAGAGAGCAACAAGAAGTTC
CTGCCATTCCAGCAGTTTGGCCGGGATATCGCCGATACCACAGACGCCGTTAGAG
ATCCCCAGACACTGGAAATCCTGGACATCACCCCTTGCAGCTTCGGCGGAGTGTC
TGTGATCACCCCTGGCACCAACACCAGCAATCAGGTGGCAGTGCTGTACCAGggc
GTGAACTGTACCGAAGTGCCCGTGGCCATTCACGCCGATCAGCTGACACCTACAT
GGCGGGTGTACTCCACCGGCAGCAATGTGTTTCAGACCAGAGCCGGCTGTCTGAT
CGGAGCCGAGtacGTGAACAATAGCTACGAGTGCGACATCCCCATCGGCGCTGGC
ATCTGTGCCAGCTACCAGACACAGACAaagAGCcacGCCTCTGTGGCCAGCCAGAG
CATCATTGCCTACACAATGTCTCTGGGCGCCGAGAACAGCGTGGCCTACTCCAAC
AACTCTATCGCTATCCCCACCAACTTCACCATCAGCGTGACCACAGAGATCCTGC
CTGTGTCCATGACCAAGACCAGCGTGGACTGCACCATGTACATCTGCGGCGATTC
CACCGAGTGCTCCAACCTGCTGCTGCAGTACGGCAGCTTCTGCACCCAGCTGaagA
GAGCCCTGACAGGGATCGCCGTGGAACAGGACAAGAACACCCAAGAGGTGTTCG
CCCAAGTGAAGCAGATCTACAAGACCCCTCCTATCAAGtacTTCGGCGGCTTCAAT
TTCAGCCAGATTCTGCCCGATCCTAGCAAGCCCAGCAAGCGGAGCcctATCGAGG
ACCTGCTGTTCAACAAAGTGACACTGGCCGACGCCGGCTTCATCAAGCAGTATG
GCGATTGTCTGGGCGACATTGCCGCCAGGGATCTGATTTGCGCCCAGAAGTTTAA
CGGACTGACAGTGCTGCCTCCTCTGCTGACCGATGAGATGATCGCCCAGTACACA
TCTGCCCTGCTGGCCGGCACAATCACAAGCGGCTGGACATTTGGAGCTGGCcctGC
TCTGCAGATCCCCTTTccaATGCAGATGGCCTACCGGTTCAACGGCATCGGAGTGA
CCCAGAATGTGCTGTACGAGAACCAGAAGCTGATCGCCAACCAGTTCAACAGCG
CCATCGGCAAGATCCAGGACAGCCTGAGCAGCACAcccAGCGCCCTGGGAAAGCT
GCAGGACGTGGTCAACcacAATGCCCAGGCACTGAACACCCTGGTCAAGCAGCTG
TCCTCCaagTTCGGCGCCATCAGCTCTGTGCTGAACGATATCCTGAGCAGACTGGA
CccccctGAAGCCGAGGTGCAGATCGACAGACTGATCACCGGAAGGCTGCAGTCCC
TGCAGACCTACGTTACCCAGCAGCTGATCAGAGCCGCCGAGATTAGAGCCTCTG
CCAATCTGGCCGCCACCAAGATGTCTGAGTGTGTGCTGGGCCAGAGCAAGAGAG
TGGACTTTTGCGGCAAGGGCTACCACCTGATGAGCTTCCCTCAGTCTGCCCCTCA
CGGCGTGGTGTTTCTGCACGTGACATACGTGCCCGCTCAAGAGAAGAATTTCACC
ACCGCTCCAGCCATCTGCCACGACGGCAAAGCCCACTTTCCTAGAGAAGGCGTG
TTCGTGTCCAACGGCACCCATTGGTTCGTGACCCAGCGGAACTTCTACGAGCCCC
AGATCATCACCACCGACAACACCTTCGTGTCTGGCAACTGCGACGTCGTGATCGG
CATTGTGAACAATACCGTGTACGACCCTCTGCAGCCCGAGCTGGACAGCTTCAAA
GAGGAACTGGATAAGTACTTTAAGAACCACACAAGCCCCGACGTGGACCTGGGC
GATATCAGCGGAATCAATGCCAGCGTCGTGAACATCCAGAAAGAGATCGACCGG
CTGAACGAGGTGGCCAAGAATCTGAACGAGAGCCTGATCGACCTGCAAGAACTG
GGGAAGTACGAGCAGTACATCAAGTGGCCCggcggaggtgggtcgCTCATAACATACA
TCGTCCTGACTATAATCAGCTTGGTATTTGGTATTTTGTCTTTGATTCTTGCA
TGCTATTTGATGTATAAACAGAAAGCTCAGCAGAAGACTCTCCTGTGGCTCG
GTAACAACACACTCGACCAGATGAGAGCAACTACAAAGATGTGATAA
Amino Acid Sequence of BQ.1.1 (Signal peptide is in bold and underlined; GS linker is
in italics; TM and CT From F is in bold and double underlined; Spike ectodomain is
underlined): SEQ ID NO: 87
MFVFLVLLPLVSSQCVNLITRTQSYTNSFTRGVYYPDKVFRSSVLHSTQDLFLPFFSN
VTWFHAISGTNGTKRFDNPVLPFNDGVYFASTEKSNIIRGWIFGTTLDSKTQSLLIVN
NATNVVIKVCEFQFCNDPFLDVYYHKNNKSWMESEFRVYSSANNCTFEYVSQPFLM
DLEGKQGNFKNLREFVFKNIDGYFKIYSKHTPINLGRDLPQGFSALEPLVDLPIGINIT
RFQTLLALHRSYLTPGDSSSGWTAGAAAYYVGYLQPRTFLLKYNENGTITDAVDCA
LDPLSETKCTLKSFTVEKGIYQTSNFRVQPTESIVRFPNITNLCPFDEVFNATTFASVY
AWNRKRISNCVADYSVLYNFAPFFAFKCYGVSPTKLNDLCFTNVYADSFVIRGNEVS
QIAPGQTGNIADYNYKLPDDFTGCVIAWNSNKLDSTVGGNYNYRYRLFRKSKLKPF
ERDISTEIYQAGNKPCNGVAGVNCYFPLQSYGFRPTYGVGHQPYRVVVLSFELLHAP
ATVCGPKKSTNLVKNKCVNFNFNGLTGTGVLTESNKKFLPFQQFGRDIADTTDAVR
DPQTLEILDITPCSFGGVSVITPGTNTSNQVAVLYQGVNCTEVPVAIHADQLTPTWRV
YSTGSNVFQTRAGCLIGAEYVNNSYECDIPIGAGICASYQTQTKSHASVASQSIIAYT
MSLGAENSVAYSNNSIAIPTNFTISVTTEILPVSMTKTSVDCTMYICGDSTECSNLLLQ
YGSFCTQLKRALTGIAVEQDKNTQEVFAQVKQIYKTPPIKYFGGFNFSQILPDPSKPS
KRSPIEDLLFNKVTLADAGFIKQYGDCLGDIAARDLICAQKFNGLTVLPPLLTDEMIA
QYTSALLAGTITSGWTFGAGPALQIPFPMQMAYRENGIGVTQNVLYENQKLIANQFN
SAIGKIQDSLSSTPSALGKLQDVVNHNAQALNTLVKQLSSKFGAISSVLNDILSRLDPP
EAEVQIDRLITGRLQSLQTYVTQQLIRAAEIRASANLAATKMSECVLGQSKRVDFCG
KGYHLMSFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKEELDKYFKN
HTSPDVDLGDISGINASVVNIQKEIDRLNEVAKNLNESLIDLQELGKYEQYIKWPGGG
GSLITYIVLTIISLVFGILSLILACYLMYKQKAQQKTLLWLGNNTLDQMRATTK
M*
Nucleotide Sequence of BQ.1.1 (Signal peptide is in bold and underlined; Spike
ectodomain is underlined): SEQ ID NO: 88
ATGTTCGTGTTTCTGGTGCTGCTGCCTCTGGTGTCCAGCCAGTGTGTGAACCT
GatcACAAGAACCCAGagcTACACCAACAGCTTTACCAGAGGCGTGTACTACCCCG
ACAAGGTGTTCAGATCCAGCGTGCTGCACTCTACCCAGGACCTGTTCCTGCCTTT
CTTCAGCAACGTGACCTGGTTCCACGCCATCTCCGGCACCAATGGCACCAAGAG
ATTCGACAACCCCGTGCTGCCCTTCAACGACGGGGTGTACTTTGCCAGCACCGAG
AAGTCCAACATCATCAGAGGCTGGATCTTCGGCACCACACTGGACAGCAAGACC
CAGAGCCTGCTGATCGTGAACAACGCCACCAACGTGGTCATCAAAGTGTGCGAG
TTCCAGTTCTGCAACGACCCCTTCCTGgacGTCTACTATCACAAGAACAACAAGAG
CTGGATGGAAAGCGAGTTCCGGGTGTACAGCAGCGCCAACAACTGCACCTTCGA
GTACGTGTCCCAGCCTTTCCTGATGGACCTGGAAGGCAAGCAGGGCAACTTCAA
GAACCTGCGCGAGTTCGTGTTCAAGAACATCGACGGCTACTTCAAGATCTACAGC
AAGCACACCCCTATCAACCTCggcCGGGATCTGCCTCAGGGCTTCTCTGCTCTGGA
ACCCCTGGTGGATCTGCCCATCGGCATCAACATCACCCGGTTTCAGACACTGCTG
GCCCTGCACAGAAGCTACCTGACACCTGGCGATAGCAGCAGCGGATGGACAGCT
GGTGCCGCCGCTTACTATGTGGGCTACCTGCAGCCTAGAACCTTTCTGCTGAAGT
ACAACGAGAACGGCACCATCACCGACGCCGTGGATTGTGCTCTGGATCCTCTGA
GCGAGACAAAGTGCACCCTGAAGTCCTTCACCGTGGAgAAGGGCATCTACCAGA
CCAGCAACTTCCGGGTGCAGCCCACCGAATCCATCGTGCGGTTCCCCAATATCAC
CAATCTGTGCCCCTTCgacGAGGTGTTCAATGCCACCaccTTCGCCTCTGTGTACGCC
TGGAACCGGAAGCGGATCAGCAATTGCGTGGCCGACTACTCCGTGCTGTACAACt
tcGCCcctTTCttcgccTTCAAGTGCTACGGCGTGTCCCCTACCAAGCTGAACGACCTGT
GCTTCACAAACGTGTACGCCGACAGCTTCGTGATCCGGGGAaacGAAGTGagcCAG
ATTGCCCCTGGACAGACAGGCaacATCGCCGACTACAACTACAAGCTGCCCGACG
ACTTCACCGGCTGTGTGATTGCCTGGAACAGCAACaagCTGGACTCCaccGTCggcGG
CAACTACAATTACcggTACCGGCTGTTCCGGAAGTCCaagCTGAAGCCCTTCGAGCG
GGACATCTCCACCGAGATCTATCAGGCCGGCaacaagCCTTGTAACGGCGTGgccGG
CgtgAACTGCTACTTCCCACTGcagTCCTACGGCTTTcggCCCACAtacGGCGTGGGCca
CCAGCCCTACAGAGTGGTGGTGCTGAGCTTCGAACTGCTGCATGCCCCTGCCACA
GTGTGCGGCCCTAAGAAgAGCACCAATCTCGTGAAGAACAAATGCGTGAACTTC
AACTTCAACGGCCTGACCGGCACCGGCGTGCTGACAGAGAGCAACAAGAAGTTC
CTGCCATTCCAGCAGTTTGGCCGGGATATCGCCGATACCACAGACGCCGTTAGAG
ATCCCCAGACACTGGAAATCCTGGACATCACCCCTTGCAGCTTCGGCGGAGTGTC
TGTGATCACCCCTGGCACCAACACCAGCAATCAGGTGGCAGTGCTGTACCAGggc
GTGAACTGTACCGAAGTGCCCGTGGCCATTCACGCCGATCAGCTGACACCTACAT
GGCGGGTGTACTCCACCGGCAGCAATGTGTTTCAGACCAGAGCCGGCTGTCTGAT
CGGAGCCGAGtacGTGAACAATAGCTACGAGTGCGACATCCCCATCGGCGCTGGC
ATCTGTGCCAGCTACCAGACACAGACAaagAGCcacGCCTCTGTGGCCAGCCAGAG
CATCATTGCCTACACAATGTCTCTGGGCGCCGAGAACAGCGTGGCCTACTCCAAC
AACTCTATCGCTATCCCCACCAACTTCACCATCAGCGTGACCACAGAGATCCTGC
CTGTGTCCATGACCAAGACCAGCGTGGACTGCACCATGTACATCTGCGGCGATTC
CACCGAGTGCTCCAACCTGCTGCTGCAGTACGGCAGCTTCTGCACCCAGCTGaagA
GAGCCCTGACAGGGATCGCCGTGGAACAGGACAAGAACACCCAAGAGGTGTTCG
CCCAAGTGAAGCAGATCTACAAGACCCCTCCTATCAAGtacTTCGGCGGCTTCAAT
TTCAGCCAGATTCTGCCCGATCCTAGCAAGCCCAGCAAGCGGAGCcctATCGAGG
ACCTGCTGTTCAACAAAGTGACACTGGCCGACGCCGGCTTCATCAAGCAGTATG
GCGATTGTCTGGGCGACATTGCCGCCAGGGATCTGATTTGCGCCCAGAAGTTTAA
CGGACTGACAGTGCTGCCTCCTCTGCTGACCGATGAGATGATCGCCCAGTACACA
TCTGCCCTGCTGGCCGGCACAATCACAAGCGGCTGGACATTTGGAGCTGGCcctGC
TCTGCAGATCCCCTTTccaATGCAGATGGCCTACCGGTTCAACGGCATCGGAGTGA
CCCAGAATGTGCTGTACGAGAACCAGAAGCTGATCGCCAACCAGTTCAACAGCG
CCATCGGCAAGATCCAGGACAGCCTGAGCAGCACAcccAGCGCCCTGGGAAAGCT
GCAGGACGTGGTCAACcacAATGCCCAGGCACTGAACACCCTGGTCAAGCAGCTG
TCCTCCaagTTCGGCGCCATCAGCTCTGTGCTGAACGATATCCTGAGCAGACTGGA
CccccctGAAGCCGAGGTGCAGATCGACAGACTGATCACCGGAAGGCTGCAGTCCC
TGCAGACCTACGTTACCCAGCAGCTGATCAGAGCCGCCGAGATTAGAGCCTCTG
CCAATCTGGCCGCCACCAAGATGTCTGAGTGTGTGCTGGGCCAGAGCAAGAGAG
TGGACTTTTGCGGCAAGGGCTACCACCTGATGAGCTTCCCTCAGTCTGCCCCTCA
CGGCGTGGTGTTTCTGCACGTGACATACGTGCCCGCTCAAGAGAAGAATTTCACC
ACCGCTCCAGCCATCTGCCACGACGGCAAAGCCCACTTTCCTAGAGAAGGCGTG
TTCGTGTCCAACGGCACCCATTGGTTCGTGACCCAGCGGAACTTCTACGAGCCCC
AGATCATCACCACCGACAACACCTTCGTGTCTGGCAACTGCGACGTCGTGATCGG
CATTGTGAACAATACCGTGTACGACCCTCTGCAGCCCGAGCTGGACAGCTTCAAA
GAGGAACTGGATAAGTACTTTAAGAACCACACAAGCCCCGACGTGGACCTGGGC
GATATCAGCGGAATCAATGCCAGCGTCGTGAACATCCAGAAAGAGATCGACCGG
CTGAACGAGGTGGCCAAGAATCTGAACGAGAGCCTGATCGACCTGCAAGAACTG
GGGAAGTACGAGCAGTACATCAAGTGGCCC
Amino Acid Sequence of BQ.1.1 (Signal peptide is in bold and underlined; Spike
ectodomain is underlined): SEQ ID NO: 89
MFVFLVLLPLVSSQCVNLITRTQSYTNSFTRGVYYPDKVFRSSVLHSTQDLFLPFFSN
VTWFHAISGTNGTKRFDNPVLPFNDGVYFASTEKSNIIRGWIFGTTLDSKTQSLLIVN
NATNVVIKVCEFQFCNDPFLDVYYHKNNKSWMESEFRVYSSANNCTFEYVSQPFLM
DLEGKQGNFKNLREFVFKNIDGYFKIYSKHTPINLGRDLPQGFSALEPLVDLPIGINIT
RFQTLLALHRSYLTPGDSSSGWTAGAAAYYVGYLQPRTFLLKYNENGTITDAVDCA
LDPLSETKCTLKSFTVEKGIYQTSNFRVQPTESIVRFPNITNLCPFDEVFNATTFASVY
AWNRKRISNCVADYSVLYNFAPFFAFKCYGVSPTKLNDLCFTNVYADSFVIRGNEVS
QIAPGQTGNIADYNYKLPDDFTGCVIAWNSNKLDSTVGGNYNYRYRLFRKSKLKPF
ERDISTEIYQAGNKPCNGVAGVNCYFPLQSYGFRPTYGVGHQPYRVVVLSFELLHAP
ATVCGPKKSTNLVKNKCVNFNFNGLTGTGVLTESNKKFLPFQQFGRDIADTTDAVR
DPQTLEILDITPCSFGGVSVITPGTNTSNQVAVLYQGVNCTEVPVAIHADQLTPTWRV
YSTGSNVFQTRAGCLIGAEYVNNSYECDIPIGAGICASYQTQTKSHASVASQSIIAYT
MSLGAENSVAYSNNSIAIPTNFTISVTTEILPVSMTKTSVDCTMYICGDSTECSNLLLQ
YGSFCTQLKRALTGIAVEQDKNTQEVFAQVKQIYKTPPIKYFGGFNFSQILPDPSKPS
KRSPIEDLLFNKVTLADAGFIKQYGDCLGDIAARDLICAQKFNGLTVLPPLLTDEMIA
QYTSALLAGTITSGWTFGAGPALQIPFPMQMAYRENGIGVTQNVLYENQKLIANQFN
SAIGKIQDSLSSTPSALGKLQDVVNHNAQALNTLVKQLSSKFGAISSVLNDILSRLDPP
EAEVQIDRLITGRLQSLQTYVTQQLIRAAEIRASANLAATKMSECVLGQSKRVDFCG
KGYHLMSFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKEELDKYFKN
HTSPDVDLGDISGINASVVNIQKEIDRLNEVAKNLNESLIDLQELGKYEQYIKWP
Nucleotide Sequence of BQ.1.1 (Spike ectodomain without signal peptide is underlined):
SEQ ID NO: 90
CAGTGTGTGAACCTGatcACAAGAACCCAGagcTACACCAACAGCTTTACCAGAGG
CGTGTACTACCCCGACAAGGTGTTCAGATCCAGCGTGCTGCACTCTACCCAGGAC
CTGTTCCTGCCTTTCTTCAGCAACGTGACCTGGTTCCACGCCATCTCCGGCACCAA
TGGCACCAAGAGATTCGACAACCCCGTGCTGCCCTTCAACGACGGGGTGTACTTT
GCCAGCACCGAGAAGTCCAACATCATCAGAGGCTGGATCTTCGGCACCACACTG
GACAGCAAGACCCAGAGCCTGCTGATCGTGAACAACGCCACCAACGTGGTCATC
AAAGTGTGCGAGTTCCAGTTCTGCAACGACCCCTTCCTGgacGTCTACTATCACAA
GAACAACAAGAGCTGGATGGAAAGCGAGTTCCGGGTGTACAGCAGCGCCAACA
ACTGCACCTTCGAGTACGTGTCCCAGCCTTTCCTGATGGACCTGGAAGGCAAGCA
GGGCAACTTCAAGAACCTGCGCGAGTTCGTGTTCAAGAACATCGACGGCTACTTC
AAGATCTACAGCAAGCACACCCCTATCAACCTCggcCGGGATCTGCCTCAGGGCTT
CTCTGCTCTGGAACCCCTGGTGGATCTGCCCATCGGCATCAACATCACCCGGTTT
CAGACACTGCTGGCCCTGCACAGAAGCTACCTGACACCTGGCGATAGCAGCAGC
GGATGGACAGCTGGTGCCGCCGCTTACTATGTGGGCTACCTGCAGCCTAGAACCT
TTCTGCTGAAGTACAACGAGAACGGCACCATCACCGACGCCGTGGATTGTGCTCT
GGATCCTCTGAGCGAGACAAAGTGCACCCTGAAGTCCTTCACCGTGGAgAAGGG
CATCTACCAGACCAGCAACTTCCGGGTGCAGCCCACCGAATCCATCGTGCGGTTC
CCCAATATCACCAATCTGTGCCCCTTCgacGAGGTGTTCAATGCCACCaccTTCGCC
TCTGTGTACGCCTGGAACCGGAAGCGGATCAGCAATTGCGTGGCCGACTACTCC
GTGCTGTACAACttcGCCcctTTCttcgccTTCAAGTGCTACGGCGTGTCCCCTACCAAG
CTGAACGACCTGTGCTTCACAAACGTGTACGCCGACAGCTTCGTGATCCGGGGAa
acGAAGTGagcCAGATTGCCCCTGGACAGACAGGCaacATCGCCGACTACAACTACA
AGCTGCCCGACGACTTCACCGGCTGTGTGATTGCCTGGAACAGCAACaagCTGGA
CTCCaccGTCggGGCAACTACAATTACcggTACCGGCTGTTCCGGAAGTCCaagCTGA
AGCCCTTCGAGCGGGACATCTCCACCGAGATCTATCAGGCCGGCaacaagCCTTGTA
ACGGCGTGgccGGCgtgAACTGCTACTTCCCACTGcagTCCTACGGCTTTcggCCCACAt
acGGCGTGGGCcacCAGCCCTACAGAGTGGTGGTGCTGAGCTTCGAACTGCTGCAT
GCCCCTGCCACAGTGTGCGGCCCTAAGAAgAGCACCAATCTCGTGAAGAACAAA
TGCGTGAACTTCAACTTCAACGGCCTGACCGGCACCGGCGTGCTGACAGAGAGC
AACAAGAAGTTCCTGCCATTCCAGCAGTTTGGCCGGGATATCGCCGATACCACA
GACGCCGTTAGAGATCCCCAGACACTGGAAATCCTGGACATCACCCCTTGCAGCT
TCGGCGGAGTGTCTGTGATCACCCCTGGCACCAACACCAGCAATCAGGTGGCAG
TGCTGTACCAGggcGTGAACTGTACCGAAGTGCCCGTGGCCATTCACGCCGATCA
GCTGACACCTACATGGCGGGTGTACTCCACCGGCAGCAATGTGTTTCAGACCAG
AGCCGGCTGTCTGATCGGAGCCGAGtacGTGAACAATAGCTACGAGTGCGACATC
CCCATCGGCGCTGGCATCTGTGCCAGCTACCAGACACAGACAaagAGCcacGCCTCT
GTGGCCAGCCAGAGCATCATTGCCTACACAATGTCTCTGGGCGCCGAGAACAGC
GTGGCCTACTCCAACAACTCTATCGCTATCCCCACCAACTTCACCATCAGCGTGA
CCACAGAGATCCTGCCTGTGTCCATGACCAAGACCAGCGTGGACTGCACCATGT
ACATCTGCGGCGATTCCACCGAGTGCTCCAACCTGCTGCTGCAGTACGGCAGCTT
CTGCACCCAGCTGaagAGAGCCCTGACAGGGATCGCCGTGGAACAGGACAAGAAC
ACCCAAGAGGTGTTCGCCCAAGTGAAGCAGATCTACAAGACCCCTCCTATCAAGt
acTTCGGCGGCTTCAATTTCAGCCAGATTCTGCCCGATCCTAGCAAGCCCAGCAAG
CGGAGCcctATCGAGGACCTGCTGTTCAACAAAGTGACACTGGCCGACGCCGGCTT
CATCAAGCAGTATGGCGATTGTCTGGGCGACATTGCCGCCAGGGATCTGATTTGC
GCCCAGAAGTTTAACGGACTGACAGTGCTGCCTCCTCTGCTGACCGATGAGATGA
TCGCCCAGTACACATCTGCCCTGCTGGCCGGCACAATCACAAGCGGCTGGACATT
TGGAGCTGGCcctGCTCTGCAGATCCCCTTTccaATGCAGATGGCCTACCGGTTCAA
CGGCATCGGAGTGACCCAGAATGTGCTGTACGAGAACCAGAAGCTGATCGCCAA
CCAGTTCAACAGCGCCATCGGCAAGATCCAGGACAGCCTGAGCAGCACAcccAGC
GCCCTGGGAAAGCTGCAGGACGTGGTCAACcacAATGCCCAGGCACTGAACACCC
TGGTCAAGCAGCTGTCCTCCaagTTCGGCGCCATCAGCTCTGTGCTGAACGATATC
CTGAGCAGACTGGACccccctGAAGCCGAGGTGCAGATCGACAGACTGATCACCGG
AAGGCTGCAGTCCCTGCAGACCTACGTTACCCAGCAGCTGATCAGAGCCGCCGA
GATTAGAGCCTCTGCCAATCTGGCCGCCACCAAGATGTCTGAGTGTGTGCTGGGC
CAGAGCAAGAGAGTGGACTTTTGCGGCAAGGGCTACCACCTGATGAGCTTCCCT
CAGTCTGCCCCTCACGGCGTGGTGTTTCTGCACGTGACATACGTGCCCGCTCAAG
AGAAGAATTTCACCACCGCTCCAGCCATCTGCCACGACGGCAAAGCCCACTTTCC
TAGAGAAGGCGTGTTCGTGTCCAACGGCACCCATTGGTTCGTGACCCAGCGGAA
CTTCTACGAGCCCCAGATCATCACCACCGACAACACCTTCGTGTCTGGCAACTGC
GACGTCGTGATCGGCATTGTGAACAATACCGTGTACGACCCTCTGCAGCCCGAGC
TGGACAGCTTCAAAGAGGAACTGGATAAGTACTTTAAGAACCACACAAGCCCCG
ACGTGGACCTGGGCGATATCAGCGGAATCAATGCCAGCGTCGTGAACATCCAGA
AAGAGATCGACCGGCTGAACGAGGTGGCCAAGAATCTGAACGAGAGCCTGATCG
ACCTGCAAGAACTGGGGAAGTACGAGCAGTACATCAAGTGGCCC
Amino Acid Sequence of BQ.1.1 (Spike ectodomain without signal peptide is
underlined): SEQ ID NO: 91
QCVNLITRTQSYTNSFTRGVYYPDKVFRSSVLHSTQDLFLPFFSNVTWFHAISGTNGT
KRFDNPVLPFNDGVYFASTEKSNIIRGWIFGTTLDSKTQSLLIVNNATNVVIKVCEFQF
CNDPFLDVYYHKNNKSWMESEFRVYSSANNCTFEYVSQPFLMDLEGKQGNFKNLR
EFVFKNIDGYFKIYSKHTPINLGRDLPQGFSALEPLVDLPIGINITRFQTLLALHRSYLT
PGDSSSGWTAGAAAYYVGYLQPRTFLLKYNENGTITDAVDCALDPLSETKCTLKSFT
VEKGIYQTSNFRVQPTESIVRFPNITNLCPFDEVFNATTFASVYAWNRKRISNCVADY
SVLYNFAPFFAFKCYGVSPTKLNDLCFTNVYADSFVIRGNEVSQIAPGQTGNIADYN
YKLPDDFTGCVIAWNSNKLDSTVGGNYNYRYRLFRKSKLKPFERDISTEIYQAGNKP
CNGVAGVNCYFPLQSYGFRPTYGVGHQPYRVVVLSFELLHAPATVCGPKKSTNLVK
NKCVNFNFNGLTGTGVLTESNKKFLPFQQFGRDIADTTDAVRDPQTLEILDITPCSFG
GVSVITPGTNTSNQVAVLYQGVNCTEVPVAIHADQLTPTWRVYSTGSNVFQTRAGC
LIGAEYVNNSYECDIPIGAGICASYQTQTKSHASVASQSIIAYTMSLGAENSVAYSNN
SIAIPTNFTISVTTEILPVSMTKTSVDCTMYICGDSTECSNLLLQYGSFCTQLKRALTGI
AVEQDKNTQEVFAQVKQIYKTPPIKYFGGFNFSQILPDPSKPSKRSPIEDLLFNKVTLA
DAGFIKQYGDCLGDIAARDLICAQKFNGLTVLPPLLTDEMIAQYTSALLAGTITSGWT
FGAGPALQIPFPMQMAYRENGIGVTQNVLYENQKLIANQFNSAIGKIQDSLSSTPSAL
GKLQDVVNHNAQALNTLVKQLSSKFGAISSVLNDILSRLDPPEAEVQIDRLITGRLQS
LQTYVTQQLIRAAEIRASANLAATKMSECVLGQSKRVDFCGKGYHLMSFPQSAPHG
VVFLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISGINA
SVVNIQKEIDRLNEVAKNLNESLIDLQELGKYEQYIKWP
Nucleotide Sequence of XBB.1.5 (Signal peptide is in bold and underlined; GS linker is
in italics; TM and CT From F is in bold and double underlined; Spike ectodomain is
underlined): SEQ ID NO: 92
ATGTTCGTGTTTCTGGTGCTGCTGCCTCTGGTGTCCAGCCAGTGTGTGAACCT
GatcACAAGAACCCAGagcTACACCAACAGCTTTACCAGAGGCGTGTACTACCCCG
ACAAGGTGTTCAGATCCAGCGTGCTGCACTCTACCCAGGACCTGTTCCTGCCTTT
CTTCAGCAACGTGACCTGGTTCCACGCCATCCACGTGTCCGGCACCAATGGCACC
AAGAGATTCGACAACCCCgctCTGCCCTTCAACGACGGGGTGTACTTTGCCAGCAC
CGAGAAGTCCAACATCATCAGAGGCTGGATCTTCGGCACCACACTGGACAGCAA
GACCCAGAGCCTGCTGATCGTGAACAACGCCACCAACGTGGTCATCAAAGTGTG
CGAGTTCCAGTTCTGCAACGACCCCTTCCTGgacGTCTATcagAAGAACAACAAGAG
CTGGATGGAAAGCGAGTTCCGGGTGTACAGCAGCGCCAACAACTGCACCTTCGA
GTACGTGTCCCAGCCTTTCCTGATGGACCTGGAAGGCAAGgagGGCAACTTCAAG
AACCTGCGCGAGTTCGTGTTCAAGAACATCGACGGCTACTTCAAGATCTACAGCA
AGCACACCCCTATCAACCTCgagCGGGATCTGCCTCAGGGCTTCTCTGCTCTGGAA
CCCCTGGTGGATCTGCCCATCGGCATCAACATCACCCGGTTTCAGACACTGCTGG
CCCTGCACAGAAGCTACCTGACACCTgtcGATAGCAGCAGCGGATGGACAGCTGG
TGCCGCCGCTTACTATGTGGGCTACCTGCAGCCTAGAACCTTTCTGCTGAAGTAC
AACGAGAACGGCACCATCACCGACGCCGTGGATTGTGCTCTGGATCCTCTGAGC
GAGACAAAGTGCACCCTGAAGTCCTTCACCGTGGAgAAGGGCATCTACCAGACC
AGCAACTTCCGGGTGCAGCCCACCGAATCCATCGTGCGGTTCCCCAATATCACCA
ATCTGTGCCCCTTCcacGAGGTGTTCAATGCCACCaccTTCGCCTCTGTGTACGCCTG
GAACCGGAAGCGGATCAGCAATTGCGTGGCCGACTACTCCGTGatcTACAACttcGC
CcctTTCttcgccTTCAAGTGCTACGGCGTGTCCCCTACCAAGCTGAACGACCTGTGCT
TCACAAACGTGTACGCCGACAGCTTCGTGATCCGGGGAaacGAAGTGagcCAGATT
GCCCCTGGACAGACAGGCaacATCGCCGACTACAACTACAAGCTGCCCGACGACT
TCACCGGCTGTGTGATTGCCTGGAACAGCAACaagCTGGACTCCAAAcccagcGGCA
ACTACAATTACCTGTACCGGCTGTTCCGGAAGTCCaagCTGAAGCCCTTCGAGCGG
GACATCTCCACCGAGATCTATCAGGCCGGCaacaagCCTTGTAACGGCGTGgccGGCc
ctAACTGCTACtctCCACTGcagTCCTACGGCTTTcggCCCACAtacGGCGTGGGCcacCAG
CCCTACAGAGTGGTGGTGCTGAGCTTCGAACTGCTGCATGCCCCTGCCACAGTGT
GCGGCCCTAAGAAgAGCACCAATCTCGTGAAGAACAAATGCGTGAACTTCAACT
TCAACGGCCTGACCGGCACCGGCGTGCTGACAGAGAGCAACAAGAAGTTCCTGC
CATTCCAGCAGTTTGGCCGGGATATCGCCGATACCACAGACGCCGTTAGAGATCC
CCAGACACTGGAAATCCTGGACATCACCCCTTGCAGCTTCGGCGGAGTGTCTGTG
ATCACCCCTGGCACCAACACCAGCAATCAGGTGGCAGTGCTGTACCAGggcGTGA
ACTGTACCGAAGTGCCCGTGGCCATTCACGCCGATCAGCTGACACCTACATGGCG
GGTGTACTCCACCGGCAGCAATGTGTTTCAGACCAGAGCCGGCTGTCTGATCGGA
GCCGAGtacGTGAACAATAGCTACGAGTGCGACATCCCCATCGGCGCTGGCATCTG
TGCCAGCTACCAGACACAGACAaagAGCcacGCCTCTGTGGCCAGCCAGAGCATCA
TTGCCTACACAATGTCTCTGGGCGCCGAGAACAGCGTGGCCTACTCCAACAACTC
TATCGCTATCCCCACCAACTTCACCATCAGCGTGACCACAGAGATCCTGCCTGTG
TCCATGACCAAGACCAGCGTGGACTGCACCATGTACATCTGCGGCGATTCCACCG
AGTGCTCCAACCTGCTGCTGCAGTACGGCAGCTTCTGCACCCAGCTGaagAGAGCC
CTGACAGGGATCGCCGTGGAACAGGACAAGAACACCCAAGAGGTGTTCGCCCAA
GTGAAGCAGATCTACAAGACCCCTCCTATCAAGtacTTCGGCGGCTTCAATTTCAG
CCAGATTCTGCCCGATCCTAGCAAGCCCAGCAAGCGGAGCcctATCGAGGACCTG
CTGTTCAACAAAGTGACACTGGCCGACGCCGGCTTCATCAAGCAGTATGGCGATT
GTCTGGGCGACATTGCCGCCAGGGATCTGATTTGCGCCCAGAAGTTTAACGGACT
GACAGTGCTGCCTCCTCTGCTGACCGATGAGATGATCGCCCAGTACACATCTGCC
CTGCTGGCCGGCACAATCACAAGCGGCTGGACATTTGGAGCTGGCcctGCTCTGCA
GATCCCCTTTccaATGCAGATGGCCTACCGGTTCAACGGCATCGGAGTGACCCAGA
ATGTGCTGTACGAGAACCAGAAGCTGATCGCCAACCAGTTCAACAGCGCCATCG
GCAAGATCCAGGACAGCCTGAGCAGCACAcccAGCGCCCTGGGAAAGCTGCAGG
ACGTGGTCAACcacAATGCCCAGGCACTGAACACCCTGGTCAAGCAGCTGTCCTCC
aagTTCGGCGCCATCAGCTCTGTGCTGAACGATATCCTGAGCAGACTGGACccccctG
AAGCCGAGGTGCAGATCGACAGACTGATCACCGGAAGGCTGCAGTCCCTGCAGA
CCTACGTTACCCAGCAGCTGATCAGAGCCGCCGAGATTAGAGCCTCTGCCAATCT
GGCCGCCACCAAGATGTCTGAGTGTGTGCTGGGCCAGAGCAAGAGAGTGGACTT
TTGCGGCAAGGGCTACCACCTGATGAGCTTCCCTCAGTCTGCCCCTCACGGCGTG
GTGTTTCTGCACGTGACATACGTGCCCGCTCAAGAGAAGAATTTCACCACCGCTC
CAGCCATCTGCCACGACGGCAAAGCCCACTTTCCTAGAGAAGGCGTGTTCGTGTC
CAACGGCACCCATTGGTTCGTGACCCAGCGGAACTTCTACGAGCCCCAGATCATC
ACCACCGACAACACCTTCGTGTCTGGCAACTGCGACGTCGTGATCGGCATTGTGA
ACAATACCGTGTACGACCCTCTGCAGCCCGAGCTGGACAGCTTCAAAGAGGAAC
TGGATAAGTACTTTAAGAACCACACAAGCCCCGACGTGGACCTGGGCGATATCA
GCGGAATCAATGCCAGCGTCGTGAACATCCAGAAAGAGATCGACCGGCTGAACG
AGGTGGCCAAGAATCTGAACGAGAGCCTGATCGACCTGCAAGAACTGGGGAAGT
ACGAGCAGTACATCAAGTGGCCCggcggaggtgggtcgCTCATAACATACATCGTCCT
GACTATAATCAGCTTGGTATTTGGTATTTTGTCTTTGATTCTTGCATGCTATT
TGATGTATAAACAGAAAGCTCAGCAGAAGACTCTCCTGTGGCTCGGTAACA
ACACACTCGACCAGATGAGAGCAACTACAAAGATGTGATAA
Amino Acid Sequence of XBB.1.5 (Signal peptide is in bold and underlined; GS linker=
is in italics; TM and CT From F is in bold and double underlined; Spike ectodomain is
underlined): SEQ ID NO: 93
MFVFLVLLPLVSSQCVNLITRTQSYTNSFTRGVYYPDKVFRSSVLHSTQDLFLPFFSN
VTWFHAIHVSGTNGTKRFDNPALPFNDGVYFASTEKSNIIRGWIFGTTLDSKTQSLLI
VNNATNVVIKVCEFQFCNDPFLDVYQKNNKSWMESEFRVYSSANNCTFEYVSQPFL
MDLEGKEGNFKNLREFVFKNIDGYFKIYSKHTPINLERDLPQGFSALEPLVDLPIGINI
TRFQTLLALHRSYLTPVDSSSGWTAGAAAYYVGYLQPRTFLLKYNENGTITDAVDC
ALDPLSETKCTLKSFTVEKGIYQTSNFRVQPTESIVRFPNITNLCPFHEVFNATTFASV
YAWNRKRISNCVADYSVIYNFAPFFAFKCYGVSPTKLNDLCFTNVYADSFVIRGNEV
SQIAPGQTGNIADYNYKLPDDFTGCVIAWNSNKLDSKPSGNYNYLYRLFRKSKLKPF
ERDISTEIYQAGNKPCNGVAGPNCYSPLQSYGFRPTYGVGHQPYRVVVLSFELLHAP
ATVCGPKKSTNLVKNKCVNFNFNGLTGTGVLTESNKKFLPFQQFGRDIADTTDAVR
DPQTLEILDITPCSFGGVSVITPGTNTSNQVAVLYQGVNCTEVPVAIHADQLTPTWRV
YSTGSNVFQTRAGCLIGAEYVNNSYECDIPIGAGICASYQTQTKSHASVASQSIIAYT
MSLGAENSVAYSNNSIAIPTNFTISVTTEILPVSMTKTSVDCTMYICGDSTECSNLLLQ
YGSFCTQLKRALTGIAVEQDKNTQEVFAQVKQIYKTPPIKYFGGFNFSQILPDPSKPS
KRSPIEDLLFNKVTLADAGFIKQYGDCLGDIAARDLICAQKFNGLTVLPPLLTDEMIA
QYTSALLAGTITSGWTFGAGPALQIPFPMQMAYRENGIGVTQNVLYENQKLIANQFN
SAIGKIQDSLSSTPSALGKLQDVVNHNAQALNTLVKQLSSKFGAISSVLNDILSRLDPP
EAEVQIDRLITGRLQSLQTYVTQQLIRAAEIRASANLAATKMSECVLGQSKRVDFCG
KGYHLMSFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKEELDKYFKN
HTSPDVDLGDISGINASVVNIQKEIDRLNEVAKNLNESLIDLQELGKYEQYIKWPGGG
GSLITYIVLTIISLVFGILSLILACYLMYKQKAQQKTLLWLGNNTLDQMRATTK
M*
Nucleotide Sequence of XBB.1.5 (Signal peptide is in bold and underlined; Spike
ectodomain is underlined): SEQ ID NO: 94
ATGTTCGTGTTTCTGGTGCTGCTGCCTCTGGTGTCCAGCCAGTGTGTGAACCT
GatcACAAGAACCCAGagcTACACCAACAGCTTTACCAGAGGCGTGTACTACCCCG
ACAAGGTGTTCAGATCCAGCGTGCTGCACTCTACCCAGGACCTGTTCCTGCCTTT
CTTCAGCAACGTGACCTGGTTCCACGCCATCCACGTGTCCGGCACCAATGGCACC
AAGAGATTCGACAACCCCgctCTGCCCTTCAACGACGGGGTGTACTTTGCCAGCAC
CGAGAAGTCCAACATCATCAGAGGCTGGATCTTCGGCACCACACTGGACAGCAA
GACCCAGAGCCTGCTGATCGTGAACAACGCCACCAACGTGGTCATCAAAGTGTG
CGAGTTCCAGTTCTGCAACGACCCCTTCCTGgacGTCTATcagAAGAACAACAAGAG
CTGGATGGAAAGCGAGTTCCGGGTGTACAGCAGCGCCAACAACTGCACCTTCGA
GTACGTGTCCCAGCCTTTCCTGATGGACCTGGAAGGCAAGgagGGCAACTTCAAG
AACCTGCGCGAGTTCGTGTTCAAGAACATCGACGGCTACTTCAAGATCTACAGCA
AGCACACCCCTATCAACCTCgagCGGGATCTGCCTCAGGGCTTCTCTGCTCTGGAA
CCCCTGGTGGATCTGCCCATCGGCATCAACATCACCCGGTTTCAGACACTGCTGG
CCCTGCACAGAAGCTACCTGACACCTgtcGATAGCAGCAGCGGATGGACAGCTGG
TGCCGCCGCTTACTATGTGGGCTACCTGCAGCCTAGAACCTTTCTGCTGAAGTAC
AACGAGAACGGCACCATCACCGACGCCGTGGATTGTGCTCTGGATCCTCTGAGC
GAGACAAAGTGCACCCTGAAGTCCTTCACCGTGGAgAAGGGCATCTACCAGACC
AGCAACTTCCGGGTGCAGCCCACCGAATCCATCGTGCGGTTCCCCAATATCACCA
ATCTGTGCCCCTTCcacGAGGTGTTCAATGCCACCaccTTCGCCTCTGTGTACGCCTG
GAACCGGAAGCGGATCAGCAATTGCGTGGCCGACTACTCCGTGatcTACAACttcGC
CcctTTCttcgccTTCAAGTGCTACGGCGTGTCCCCTACCAAGCTGAACGACCTGTGCT
TCACAAACGTGTACGCCGACAGCTTCGTGATCCGGGGAaacGAAGTGagcCAGATT
GCCCCTGGACAGACAGGCaacATCGCCGACTACAACTACAAGCTGCCCGACGACT
TCACCGGCTGTGTGATTGCCTGGAACAGCAACaagCTGGACTCCAAAcccagcGGCA
ACTACAATTACCTGTACCGGCTGTTCCGGAAGTCCaagCTGAAGCCCTTCGAGCGG
GACATCTCCACCGAGATCTATCAGGCCGGCaacaagCCTTGTAACGGCGTGgccGGCc
ctAACTGCTACtctCCACTGcagTCCTACGGCTTTcggCCCACAtacGGCGTGGGCcacCAG
CCCTACAGAGTGGTGGTGCTGAGCTTCGAACTGCTGCATGCCCCTGCCACAGTGT
GCGGCCCTAAGAAgAGCACCAATCTCGTGAAGAACAAATGCGTGAACTTCAACT
TCAACGGCCTGACCGGCACCGGCGTGCTGACAGAGAGCAACAAGAAGTTCCTGC
CATTCCAGCAGTTTGGCCGGGATATCGCCGATACCACAGACGCCGTTAGAGATCC
CCAGACACTGGAAATCCTGGACATCACCCCTTGCAGCTTCGGCGGAGTGTCTGTG
ATCACCCCTGGCACCAACACCAGCAATCAGGTGGCAGTGCTGTACCAGggcGTGA
ACTGTACCGAAGTGCCCGTGGCCATTCACGCCGATCAGCTGACACCTACATGGCG
GGTGTACTCCACCGGCAGCAATGTGTTTCAGACCAGAGCCGGCTGTCTGATCGGA
GCCGAGtacGTGAACAATAGCTACGAGTGCGACATCCCCATCGGCGCTGGCATCTG
TGCCAGCTACCAGACACAGACAaagAGCcacGCCTCTGTGGCCAGCCAGAGCATCA
TTGCCTACACAATGTCTCTGGGCGCCGAGAACAGCGTGGCCTACTCCAACAACTC
TATCGCTATCCCCACCAACTTCACCATCAGCGTGACCACAGAGATCCTGCCTGTG
TCCATGACCAAGACCAGCGTGGACTGCACCATGTACATCTGCGGCGATTCCACCG
AGTGCTCCAACCTGCTGCTGCAGTACGGCAGCTTCTGCACCCAGCTGaagAGAGCC
CTGACAGGGATCGCCGTGGAACAGGACAAGAACACCCAAGAGGTGTTCGCCCAA
GTGAAGCAGATCTACAAGACCCCTCCTATCAAGtacTTCGGCGGCTTCAATTTCAG
CCAGATTCTGCCCGATCCTAGCAAGCCCAGCAAGCGGAGCcctATCGAGGACCTG
CTGTTCAACAAAGTGACACTGGCCGACGCCGGCTTCATCAAGCAGTATGGCGATT
GTCTGGGCGACATTGCCGCCAGGGATCTGATTTGCGCCCAGAAGTTTAACGGACT
GACAGTGCTGCCTCCTCTGCTGACCGATGAGATGATCGCCCAGTACACATCTGCC
CTGCTGGCCGGCACAATCACAAGCGGCTGGACATTTGGAGCTGGCcctGCTCTGCA
GATCCCCTTTccaATGCAGATGGCCTACCGGTTCAACGGCATCGGAGTGACCCAGA
ATGTGCTGTACGAGAACCAGAAGCTGATCGCCAACCAGTTCAACAGCGCCATCG
GCAAGATCCAGGACAGCCTGAGCAGCACAcccAGCGCCCTGGGAAAGCTGCAGG
ACGTGGTCAACcacAATGCCCAGGCACTGAACACCCTGGTCAAGCAGCTGTCCTCC
aagTTCGGCGCCATCAGCTCTGTGCTGAACGATATCCTGAGCAGACTGGACccccctG
AAGCCGAGGTGCAGATCGACAGACTGATCACCGGAAGGCTGCAGTCCCTGCAGA
CCTACGTTACCCAGCAGCTGATCAGAGCCGCCGAGATTAGAGCCTCTGCCAATCT
GGCCGCCACCAAGATGTCTGAGTGTGTGCTGGGCCAGAGCAAGAGAGTGGACTT
TTGCGGCAAGGGCTACCACCTGATGAGCTTCCCTCAGTCTGCCCCTCACGGCGTG
GTGTTTCTGCACGTGACATACGTGCCCGCTCAAGAGAAGAATTTCACCACCGCTC
CAGCCATCTGCCACGACGGCAAAGCCCACTTTCCTAGAGAAGGCGTGTTCGTGTC
CAACGGCACCCATTGGTTCGTGACCCAGCGGAACTTCTACGAGCCCCAGATCATC
ACCACCGACAACACCTTCGTGTCTGGCAACTGCGACGTCGTGATCGGCATTGTGA
ACAATACCGTGTACGACCCTCTGCAGCCCGAGCTGGACAGCTTCAAAGAGGAAC
TGGATAAGTACTTTAAGAACCACACAAGCCCCGACGTGGACCTGGGCGATATCA
GCGGAATCAATGCCAGCGTCGTGAACATCCAGAAAGAGATCGACCGGCTGAACG
AGGTGGCCAAGAATCTGAACGAGAGCCTGATCGACCTGCAAGAACTGGGGAAGT
ACGAGCAGTACATCAAGTGGCCC
Amino Acid Sequence of XBB.1.5 (Signal peptide is in bold and underlined; Spike
ectodomain is underlined): SEQ ID NO: 95
MFVFLVLLPLVSSQCVNLITRTQSYTNSFTRGVYYPDKVFRSSVLHSTQDLFLPFFSN
VTWFHAIHVSGTNGTKRFDNPALPENDGVYFASTEKSNIIRGWIFGTTLDSKTQSLLI
VNNATNVVIKVCEFQFCNDPFLDVYQKNNKSWMESEFRVYSSANNCTFEYVSQPFL
MDLEGKEGNFKNLREFVFKNIDGYFKIYSKHTPINLERDLPQGFSALEPLVDLPIGINI
TRFQTLLALHRSYLTPVDSSSGWTAGAAAYYVGYLQPRTFLLKYNENGTITDAVDC
ALDPLSETKCTLKSFTVEKGIYQTSNFRVQPTESIVRFPNITNLCPFHEVFNATTFASV
YAWNRKRISNCVADYSVIYNFAPFFAFKCYGVSPTKLNDLCFTNVYADSFVIRGNEV
SQIAPGQTGNIADYNYKLPDDFTGCVIAWNSNKLDSKPSGNYNYLYRLFRKSKLKPF
ERDISTEIYQAGNKPCNGVAGPNCYSPLQSYGFRPTYGVGHQPYRVVVLSFELLHAP
ATVCGPKKSTNLVKNKCVNFNFNGLTGTGVLTESNKKFLPFQQFGRDIADTTDAVR
DPQTLEILDITPCSFGGVSVITPGTNTSNQVAVLYQGVNCTEVPVAIHADQLTPTWRV
YSTGSNVFQTRAGCLIGAEYVNNSYECDIPIGAGICASYQTQTKSHASVASQSIIAYT
MSLGAENSVAYSNNSIAIPTNFTISVTTEILPVSMTKTSVDCTMYICGDSTECSNLLLQ
YGSFCTQLKRALTGIAVEQDKNTQEVFAQVKQIYKTPPIKYFGGFNFSQILPDPSKPS
KRSPIEDLLFNKVTLADAGFIKQYGDCLGDIAARDLICAQKFNGLTVLPPLLTDEMIA
QYTSALLAGTITSGWTFGAGPALQIPFPMQMAYRENGIGVTQNVLYENQKLIANQFN
SAIGKIQDSLSSTPSALGKLQDVVNHNAQALNTLVKQLSSKFGAISSVLNDILSRLDPP
EAEVQIDRLITGRLQSLQTYVTQQLIRAAEIRASANLAATKMSECVLGQSKRVDFCG
KGYHLMSFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKEELDKYFKN
HTSPDVDLGDISGINASVVNIQKEIDRLNEVAKNLNESLIDLQELGKYEQYIKWP
Nucleotide Sequence of XBB.1.5 (Spike ectodomain without signal peptide is
underlined): SEQ ID NO: 96
CAGTGTGTGAACCTGatcACAAGAACCCAGagcTACACCAACAGCTTTACCAGAGG
CGTGTACTACCCCGACAAGGTGTTCAGATCCAGCGTGCTGCACTCTACCCAGGAC
CTGTTCCTGCCTTTCTTCAGCAACGTGACCTGGTTCCACGCCATCCACGTGTCCGG
CACCAATGGCACCAAGAGATTCGACAACCCCgctCTGCCCTTCAACGACGGGGTGT
ACTTTGCCAGCACCGAGAAGTCCAACATCATCAGAGGCTGGATCTTCGGCACCA
CACTGGACAGCAAGACCCAGAGCCTGCTGATCGTGAACAACGCCACCAACGTGG
TCATCAAAGTGTGCGAGTTCCAGTTCTGCAACGACCCCTTCCTGgacGTCTATcagA
AGAACAACAAGAGCTGGATGGAAAGCGAGTTCCGGGTGTACAGCAGCGCCAAC
AACTGCACCTTCGAGTACGTGTCCCAGCCTTTCCTGATGGACCTGGAAGGCAAGg
agGGCAACTTCAAGAACCTGCGCGAGTTCGTGTTCAAGAACATCGACGGCTACTT
CAAGATCTACAGCAAGCACACCCCTATCAACCTCgagCGGGATCTGCCTCAGGGC
TTCTCTGCTCTGGAACCCCTGGTGGATCTGCCCATCGGCATCAACATCACCCGGT
TTCAGACACTGCTGGCCCTGCACAGAAGCTACCTGACACCTgtcGATAGCAGCAGC
GGATGGACAGCTGGTGCCGCCGCTTACTATGTGGGCTACCTGCAGCCTAGAACCT
TTCTGCTGAAGTACAACGAGAACGGCACCATCACCGACGCCGTGGATTGTGCTCT
GGATCCTCTGAGCGAGACAAAGTGCACCCTGAAGTCCTTCACCGTGGAgAAGGG
CATCTACCAGACCAGCAACTTCCGGGTGCAGCCCACCGAATCCATCGTGCGGTTC
CCCAATATCACCAATCTGTGCCCCTTCcacGAGGTGTTCAATGCCACCaccTTCGCCT
CTGTGTACGCCTGGAACCGGAAGCGGATCAGCAATTGCGTGGCCGACTACTCCG
TGatcTACAACttcGCCcctTTCttcgccTTCAAGTGCTACGGCGTGTCCCCTACCAAGCTG
AACGACCTGTGCTTCACAAACGTGTACGCCGACAGCTTCGTGATCCGGGGAaacG
AAGTGagcCAGATTGCCCCTGGACAGACAGGCaacATCGCCGACTACAACTACAAG
CTGCCCGACGACTTCACCGGCTGTGTGATTGCCTGGAACAGCAACaagCTGGACTC
CAAAcccagcGGCAACTACAATTACCTGTACCGGCTGTTCCGGAAGTCCaagCTGAAG
CCCTTCGAGCGGGACATCTCCACCGAGATCTATCAGGCCGGCaacaagCCTTGTAAC
GGCGTGgccGGCcctAACTGCTACtctCCACTGcagTCCTACGGCTTTcggCCCACAtacGG
CGTGGGCcacCAGCCCTACAGAGTGGTGGTGCTGAGCTTCGAACTGCTGCATGCCC
CTGCCACAGTGTGCGGCCCTAAGAAgAGCACCAATCTCGTGAAGAACAAATGCG
TGAACTTCAACTTCAACGGCCTGACCGGCACCGGCGTGCTGACAGAGAGCAACA
AGAAGTTCCTGCCATTCCAGCAGTTTGGCCGGGATATCGCCGATACCACAGACGC
CGTTAGAGATCCCCAGACACTGGAAATCCTGGACATCACCCCTTGCAGCTTCGGC
GGAGTGTCTGTGATCACCCCTGGCACCAACACCAGCAATCAGGTGGCAGTGCTG
TACCAGggcGTGAACTGTACCGAAGTGCCCGTGGCCATTCACGCCGATCAGCTGA
CACCTACATGGCGGGTGTACTCCACCGGCAGCAATGTGTTTCAGACCAGAGCCG
GCTGTCTGATCGGAGCCGAGtacGTGAACAATAGCTACGAGTGCGACATCCCCATC
GGCGCTGGCATCTGTGCCAGCTACCAGACACAGACAaagAGCcacGCCTCTGTGGC
CAGCCAGAGCATCATTGCCTACACAATGTCTCTGGGCGCCGAGAACAGCGTGGC
CTACTCCAACAACTCTATCGCTATCCCCACCAACTTCACCATCAGCGTGACCACA
GAGATCCTGCCTGTGTCCATGACCAAGACCAGCGTGGACTGCACCATGTACATCT
GCGGCGATTCCACCGAGTGCTCCAACCTGCTGCTGCAGTACGGCAGCTTCTGCAC
CCAGCTGaagAGAGCCCTGACAGGGATCGCCGTGGAACAGGACAAGAACACCCA
AGAGGTGTTCGCCCAAGTGAAGCAGATCTACAAGACCCCTCCTATCAAGtacTTCG
GCGGCTTCAATTTCAGCCAGATTCTGCCCGATCCTAGCAAGCCCAGCAAGCGGA
GCcctATCGAGGACCTGCTGTTCAACAAAGTGACACTGGCCGACGCCGGCTTCATC
AAGCAGTATGGCGATTGTCTGGGCGACATTGCCGCCAGGGATCTGATTTGCGCCC
AGAAGTTTAACGGACTGACAGTGCTGCCTCCTCTGCTGACCGATGAGATGATCGC
CCAGTACACATCTGCCCTGCTGGCCGGCACAATCACAAGCGGCTGGACATTTGG
AGCTGGCcctGCTCTGCAGATCCCCTTTccaATGCAGATGGCCTACCGGTTCAACGG
CATCGGAGTGACCCAGAATGTGCTGTACGAGAACCAGAAGCTGATCGCCAACCA
GTTCAACAGCGCCATCGGCAAGATCCAGGACAGCCTGAGCAGCACAcccAGCGCC
CTGGGAAAGCTGCAGGACGTGGTCAACcacAATGCCCAGGCACTGAACACCCTGG
TCAAGCAGCTGTCCTCCaagTTCGGCGCCATCAGCTCTGTGCTGAACGATATCCTG
AGCAGACTGGACccccctGAAGCCGAGGTGCAGATCGACAGACTGATCACCGGAAG
GCTGCAGTCCCTGCAGACCTACGTTACCCAGCAGCTGATCAGAGCCGCCGAGATT
AGAGCCTCTGCCAATCTGGCCGCCACCAAGATGTCTGAGTGTGTGCTGGGCCAG
AGCAAGAGAGTGGACTTTTGCGGCAAGGGCTACCACCTGATGAGCTTCCCTCAG
TCTGCCCCTCACGGCGTGGTGTTTCTGCACGTGACATACGTGCCCGCTCAAGAGA
AGAATTTCACCACCGCTCCAGCCATCTGCCACGACGGCAAAGCCCACTTTCCTAG
AGAAGGCGTGTTCGTGTCCAACGGCACCCATTGGTTCGTGACCCAGCGGAACTTC
TACGAGCCCCAGATCATCACCACCGACAACACCTTCGTGTCTGGCAACTGCGACG
TCGTGATCGGCATTGTGAACAATACCGTGTACGACCCTCTGCAGCCCGAGCTGGA
CAGCTTCAAAGAGGAACTGGATAAGTACTTTAAGAACCACACAAGCCCCGACGT
GGACCTGGGCGATATCAGCGGAATCAATGCCAGCGTCGTGAACATCCAGAAAGA
GATCGACCGGCTGAACGAGGTGGCCAAGAATCTGAACGAGAGCCTGATCGACCT
GCAAGAACTGGGGAAGTACGAGCAGTACATCAAGTGGCCC
Amino Acid Sequence of XBB.1.5 (Spike ectodomain without signal peptide is
underlined): SEQ ID NO: 97
QCVNLITRTQSYTNSFTRGVYYPDKVFRSSVLHSTQDLFLPFFSNVTWFHAIHVSGTN
GTKRFDNPALPENDGVYFASTEKSNIIRGWIFGTTLDSKTQSLLIVNNATNVVIKVCEF
QFCNDPFLDVYQKNNKSWMESEFRVYSSANNCTFEYVSQPFLMDLEGKEGNFKNLR
EFVFKNIDGYFKIYSKHTPINLERDLPQGFSALEPLVDLPIGINITRFQTLLALHRSYLT
PVDSSSGWTAGAAAYYVGYLQPRTFLLKYNENGTITDAVDCALDPLSETKCTLKSFT
VEKGIYQTSNERVQPTESIVRFPNITNLCPFHEVFNATTFASVYAWNRKRISNCVADY
SVIYNFAPFFAFKCYGVSPTKLNDLCFTNVYADSFVIRGNEVSQIAPGQTGNIADYNY
KLPDDFTGCVIAWNSNKLDSKPSGNYNYLYRLFRKSKLKPFERDISTEIYQAGNKPC
NGVAGPNCYSPLQSYGFRPTYGVGHQPYRVVVLSFELLHAPATVCGPKKSTNLVKN
KCVNFNFNGLTGTGVLTESNKKFLPFQQFGRDIADTTDAVRDPQTLEILDITPCSFGG
VSVITPGTNTSNQVAVLYQGVNCTEVPVAIHADQLTPTWRVYSTGSNVFQTRAGCLI
GAEYVNNSYECDIPIGAGICASYQTQTKSHASVASQSIIAYTMSLGAENSVAYSNNSI
AIPTNFTISVTTEILPVSMTKTSVDCTMYICGDSTECSNLLLQYGSFCTQLKRALTGIA
VEQDKNTQEVFAQVKQIYKTPPIKYFGGFNFSQILPDPSKPSKRSPIEDLLFNKVTLAD
AGFIKQYGDCLGDIAARDLICAQKFNGLTVLPPLLTDEMIAQYTSALLAGTITSGWTF
GAGPALQIPFPMQMAYRENGIGVTQNVLYENQKLIANQFNSAIGKIQDSLSSTPSALG
KLQDVVNHNAQALNTLVKQLSSKFGAISSVLNDILSRLDPPEAEVQIDRLITGRLQSL
QTYVTQQLIRAAEIRASANLAATKMSECVLGQSKRVDFCGKGYHLMSFPQSAPHGV
VFLHVTYVPAQEKNFTTAPAICHDGKAHFPREGVFVSNGTHWFVTQRNFYEPQIITT
DNTFVSGNCDVVIGIVNNTVYDPLQPELDSFKEELDKYFKNHTSPDVDLGDISGINAS
VVNIQKEIDRLNEVAKNLNESLIDLQELGKYEQYIKWP
Nucleotide Sequence of BA.1 S371 S375 (Signal peptide is in bold and underlined; GS
linker is in italics; transmembrane and cytoplasmic domains of NDV F protein are in
bold and double underlined; HXP-S ectodomain is underlined; S371 S375 is in bold,
italics and underlined): SEQ ID NO: 98
ATGTTCGTGTTTCTGGTGCTGCTGCCTCTGGTGTCCAGCCAGTGTGTGAACCT
GACCACAAGAACCCAGCTGCCTCCAGCCTACACCAACAGCTTTACCAGAGGCGT
GTACTACCCCGACAAGGTGTTCAGATCCAGCGTGCTGCACTCTACCCAGGACCTG
TTCCTGCCTTTCTTCAGCAACGTGACCTGGTTCCACgtgATCTCCGGCACCAATGGC
ACCAAGAGATTCGACAACCCCGTGCTGCCCTTCAACGACGGGGTGTACTTTGCCA
GCatcGAGAAGTCCAACATCATCAGAGGCTGGATCTTCGGCACCACACTGGACAG
CAAGACCCAGAGCCTGCTGATCGTGAACAACGCCACCAACGTGGTCATCAAAGT
GTGCGAGTTCCAGTTCTGCAACGACCCCTTCCTGgacCACAAGAACAACAAGAGC
TGGATGGAAAGCGAGTTCCGGGTGTACAGCAGCGCCAACAACTGCACCTTCGAG
TACGTGTCCCAGCCTTTCCTGATGGACCTGGAAGGCAAGCAGGGCAACTTCAAG
AACCTGCGCGAGTTCGTGTTCAAGAACATCGACGGCTACTTCAAGATCTACAGCA
AGCACACCCCTATCatcGTGCGGgaacccgaaGATCTGCCTCAGGGCTTCTCTGCTCTG
GAACCCCTGGTGGATCTGCCCATCGGCATCAACATCACCCGGTTTCAGACACTGC
TGGCCCTGCACAGAAGCTACCTGACACCTGGCGATAGCAGCAGCGGATGGACAG
CTGGTGCCGCCGCTTACTATGTGGGCTACCTGCAGCCTAGAACCTTTCTGCTGAA
GTACAACGAGAACGGCACCATCACCGACGCCGTGGATTGTGCTCTGGATCCTCTG
AGCGAGACAAAGTGCACCCTGAAGTCCTTCACCGTGGAgAAGGGCATCTACCAG
ACCAGCAACTTCCGGGTGCAGCCCACCGAATCCATCGTGCGGTTCCCCAATATCA
CCAATCTGTGCCCCTTCgacGAGGTGTTCAATGCCACCAGATTCGCCTCTGTGTAC
GCCTGGAACCGGAAGCGGATCAGCAATTGCGTGGCCGACTACTCCGTGCTGTAC
AACtccGCCcctTTCagcACCTTCAAGTGCTACGGCGTGTCCCCTACCAAGCTGAACG
ACCTGTGCTTCACAAACGTGTACGCCGACAGCTTCGTGATCCGGGGAGATGAAG
TGCGGCAGATTGCCCCTGGACAGACAGGCaacATCGCCGACTACAACTACAAGCT
GCCCGACGACTTCACCGGCTGTGTGATTGCCTGGAACAGCAACaagCTGGACTCCA
AAGTCagcGGCAACTACAATTACCTGTACCGGCTGTTCCGGAAGTCCAATCTGAAG
CCCTTCGAGCGGGACATCTCCACCGAGATCTATCAGGCCGGCaacaagCCTTGTAAC
GGCGTGgccGGCTTCAACTGCTACTTCCCACTGagaTCCTACagcTTTagaCCCACAtacG
GCGTGGGCcacCAGCCCTACAGAGTGGTGGTGCTGAGCTTCGAACTGCTGCATGCC
CCTGCCACAGTGTGCGGCCCTAAGAAgAGCACCAATCTCGTGAAGAACAAATGC
GTGAACTTCAACTTCAACGGCCTGaagGGCACCGGCGTGCTGACAGAGAGCAACA
AGAAGTTCCTGCCATTCCAGCAGTTTGGCCGGGATATCGCCGATACCACAGACGC
CGTTAGAGATCCCCAGACACTGGAAATCCTGGACATCACCCCTTGCAGCTTCGGC
GGAGTGTCTGTGATCACCCCTGGCACCAACACCAGCAATCAGGTGGCAGTGCTG
TACCAGggcGTGAACTGTACCGAAGTGCCCGTGGCCATTCACGCCGATCAGCTGA
CACCTACATGGCGGGTGTACTCCACCGGCAGCAATGTGTTTCAGACCAGAGCCG
GCTGTCTGATCGGAGCCGAGtacGTGAACAATAGCTACGAGTGCGACATCCCCATC
GGCGCTGGCATCTGTGCCAGCTACCAGACACAGACAaagAGCcacGCCTCTGTGGC
CAGCCAGAGCATCATTGCCTACACAATGTCTCTGGGCGCCGAGAACAGCGTGGC
CTACTCCAACAACTCTATCGCTATCCCCACCAACTTCACCATCAGCGTGACCACA
GAGATCCTGCCTGTGTCCATGACCAAGACCAGCGTGGACTGCACCATGTACATCT
GCGGCGATTCCACCGAGTGCTCCAACCTGCTGCTGCAGTACGGCAGCTTCTGCAC
CCAGCTGaagAGAGCCCTGACAGGGATCGCCGTGGAACAGGACAAGAACACCCA
AGAGGTGTTCGCCCAAGTGAAGCAGATCTACAAGACCCCTCCTATCAAGtacTTCG
GCGGCTTCAATTTCAGCCAGATTCTGCCCGATCCTAGCAAGCCCAGCAAGCGGA
GCcctATCGAGGACCTGCTGTTCAACAAAGTGACACTGGCCGACGCCGGCTTCATC
AAGCAGTATGGCGATTGTCTGGGCGACATTGCCGCCAGGGATCTGATTTGCGCCC
AGAAGTTTaagGGACTGACAGTGCTGCCTCCTCTGCTGACCGATGAGATGATCGCC
CAGTACACATCTGCCCTGCTGGCCGGCACAATCACAAGCGGCTGGACATTTGGA
GCTGGCcctGCTCTGCAGATCCCCTTTccaATGCAGATGGCCTACCGGTTCAACGGC
ATCGGAGTGACCCAGAATGTGCTGTACGAGAACCAGAAGCTGATCGCCAACCAG
TTCAACAGCGCCATCGGCAAGATCCAGGACAGCCTGAGCAGCACAcccAGCGCCC
TGGGAAAGCTGCAGGACGTGGTCAACcacAATGCCCAGGCACTGAACACCCTGGT
CAAGCAGCTGTCCTCCaagTTCGGCGCCATCAGCTCTGTGCTGAACGATATCttcAG
CAGACTGGACccccctGAAGCCGAGGTGCAGATCGACAGACTGATCACCGGAAGGC
TGCAGTCCCTGCAGACCTACGTTACCCAGCAGCTGATCAGAGCCGCCGAGATTA
GAGCCTCTGCCAATCTGGCCGCCACCAAGATGTCTGAGTGTGTGCTGGGCCAGA
GCAAGAGAGTGGACTTTTGCGGCAAGGGCTACCACCTGATGAGCTTCCCTCAGTC
TGCCCCTCACGGCGTGGTGTTTCTGCACGTGACATACGTGCCCGCTCAAGAGAAG
AATTTCACCACCGCTCCAGCCATCTGCCACGACGGCAAAGCCCACTTTCCTAGAG
AAGGCGTGTTCGTGTCCAACGGCACCCATTGGTTCGTGACCCAGCGGAACTTCTA
CGAGCCCCAGATCATCACCACCGACAACACCTTCGTGTCTGGCAACTGCGACGTC
GTGATCGGCATTGTGAACAATACCGTGTACGACCCTCTGCAGCCCGAGCTGGAC
AGCTTCAAAGAGGAACTGGATAAGTACTTTAAGAACCACACAAGCCCCGACGTG
GACCTGGGCGATATCAGCGGAATCAATGCCAGCGTCGTGAACATCCAGAAAGAG
ATCGACCGGCTGAACGAGGTGGCCAAGAATCTGAACGAGAGCCTGATCGACCTG
CAAGAACTGGGGAAGTACGAGCAGTACATCAAGTGGCCCggcggaggtgggtcgCTCAT
AACATACATCGTCCTGACTATAATCAGCTTGGTATTTGGTATTTTGTCTTTGA
TTCTTGCATGCTATTTGATGTATAAACAGAAAGCTCAGCAGAAGACTCTCCT
GTGGCTCGGTAACAACACACTCGACCAGATGAGAGCAACTACAAAGATGTG
Amino Acid Sequence of BA.1 S371 S375 (Signal peptide is in bold and underlined; GS
linker is in italics; transmembrane and cytoplasmic domains of NDV F protein are in
bold and double underlined; HXP-S ectodomain is underlined; S371 S375 is in bold,
italics and underlined): SEQ ID NO: 99
MFVFLVLLPLVSSQCVNLTTRTQLPPAYTNSFTRGVYYPDKVFRSSVLHSTQDLFLP
FFSNVTWFHVISGTNGTKRFDNPVLPFNDGVYFASIEKSNIIRGWIFGTTLDSKTQSLL
IVNNATNVVIKVCEFQFCNDPFLDHKNNKSWMESEFRVYSSANNCTFEYVSQPFLM
DLEGKQGNFKNLREFVFKNIDGYFKIYSKHTPIIVREPEDLPQGFSALEPLVDLPIGINI
TRFQTLLALHRSYLTPGDSSSGWTAGAAAYYVGYLQPRTFLLKYNENGTITDAVDC
ALDPLSETKCTLKSFTVEKGIYQTSNFRVQPTESIVRFPNITNLCPFDEVFNATRFASV
YAWNRKRISNCVADYSVLYNSAPFSTFKCYGVSPTKLNDLCFTNVYADSFVIRGDEV
RQIAPGQTGNIADYNYKLPDDFTGCVIAWNSNKLDSKVSGNYNYLYRLFRKSNLKPF
ERDISTEIYQAGNKPCNGVAGFNCYFPLRSYSFRPTYGVGHQPYRVVVLSFELLHAP
ATVCGPKKSTNLVKNKCVNFNFNGLKGTGVLTESNKKFLPFQQFGRDIADTTDAVR
DPQTLEILDITPCSFGGVSVITPGTNTSNQVAVLYQGVNCTEVPVAIHADQLTPTWRV
YSTGSNVFQTRAGCLIGAEYVNNSYECDIPIGAGICASYQTQTKSHASVASQSIIAYT
MSLGAENSVAYSNNSIAIPTNFTISVTTEILPVSMTKTSVDCTMYICGDSTECSNLLLQ
YGSFCTQLKRALTGIAVEQDKNTQEVFAQVKQIYKTPPIKYFGGFNFSQILPDPSKPS
KRSPIEDLLFNKVTLADAGFIKQYGDCLGDIAARDLICAQKFKGLTVLPPLLTDEMIA
QYTSALLAGTITSGWTFGAGPALQIPFPMQMAYRENGIGVTQNVLYENQKLIANQFN
SAIGKIQDSLSSTPSALGKLQDVVNHNAQALNTLVKQLSSKFGAISSVLNDIFSRLDPP
EAEVQIDRLITGRLQSLQTYVTQQLIRAAEIRASANLAATKMSECVLGQSKRVDFCG
KGYHLMSFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKEELDKYFKN
HTSPDVDLGDISGINASVVNIQKEIDRLNEVAKNLNESLIDLQELGKYEQYIKWPGGG
GSLITYIVLTIISLVFGILSLILACYLMYKQKAQQKTLLWLGNNTLDQMRATTK
M*
Nucleotide Sequence of BA.1 S371 S375 (Signal peptide are in bold and underlined;
HXP-S ectodomain is underlined; S371 S375 is in bold, italics and underlined): SEQ ID
NO: 100
ATGTTCGTGTTTCTGGTGCTGCTGCCTCTGGTGTCCAGCCAGTGTGTGAACCT
GACCACAAGAACCCAGCTGCCTCCAGCCTACACCAACAGCTTTACCAGAGGCGT
GTACTACCCCGACAAGGTGTTCAGATCCAGCGTGCTGCACTCTACCCAGGACCTG
TTCCTGCCTTTCTTCAGCAACGTGACCTGGTTCCACgtgATCTCCGGCACCAATGGC
ACCAAGAGATTCGACAACCCCGTGCTGCCCTTCAACGACGGGGTGTACTTTGCCA
GCatcGAGAAGTCCAACATCATCAGAGGCTGGATCTTCGGCACCACACTGGACAG
CAAGACCCAGAGCCTGCTGATCGTGAACAACGCCACCAACGTGGTCATCAAAGT
GTGCGAGTTCCAGTTCTGCAACGACCCCTTCCTGgacCACAAGAACAACAAGAGC
TGGATGGAAAGCGAGTTCCGGGTGTACAGCAGCGCCAACAACTGCACCTTCGAG
TACGTGTCCCAGCCTTTCCTGATGGACCTGGAAGGCAAGCAGGGCAACTTCAAG
AACCTGCGCGAGTTCGTGTTCAAGAACATCGACGGCTACTTCAAGATCTACAGCA
AGCACACCCCTATCatcGTGCGGgaacccgaaGATCTGCCTCAGGGCTTCTCTGCTCTG
GAACCCCTGGTGGATCTGCCCATCGGCATCAACATCACCCGGTTTCAGACACTGC
TGGCCCTGCACAGAAGCTACCTGACACCTGGCGATAGCAGCAGCGGATGGACAG
CTGGTGCCGCCGCTTACTATGTGGGCTACCTGCAGCCTAGAACCTTTCTGCTGAA
GTACAACGAGAACGGCACCATCACCGACGCCGTGGATTGTGCTCTGGATCCTCTG
AGCGAGACAAAGTGCACCCTGAAGTCCTTCACCGTGGAgAAGGGCATCTACCAG
ACCAGCAACTTCCGGGTGCAGCCCACCGAATCCATCGTGCGGTTCCCCAATATCA
CCAATCTGTGCCCCTTCgacGAGGTGTTCAATGCCACCAGATTCGCCTCTGTGTAC
GCCTGGAACCGGAAGCGGATCAGCAATTGCGTGGCCGACTACTCCGTGCTGTAC
AACtccGCCcctTTCagcACCTTCAAGTGCTACGGCGTGTCCCCTACCAAGCTGAACG
ACCTGTGCTTCACAAACGTGTACGCCGACAGCTTCGTGATCCGGGGAGATGAAG
TGCGGCAGATTGCCCCTGGACAGACAGGCaacATCGCCGACTACAACTACAAGCT
GCCCGACGACTTCACCGGCTGTGTGATTGCCTGGAACAGCAACaagCTGGACTCCA
AAGTCagcGGCAACTACAATTACCTGTACCGGCTGTTCCGGAAGTCCAATCTGAAG
CCCTTCGAGCGGGACATCTCCACCGAGATCTATCAGGCCGGCaacaagCCTTGTAAC
GGCGTGgccGGCTTCAACTGCTACTTCCCACTGagaTCCTACagcTTTagaCCCACAtacG
GCGTGGGCcacCAGCCCTACAGAGTGGTGGTGCTGAGCTTCGAACTGCTGCATGCC
CCTGCCACAGTGTGCGGCCCTAAGAAgAGCACCAATCTCGTGAAGAACAAATGC
GTGAACTTCAACTTCAACGGCCTGaagGGCACCGGCGTGCTGACAGAGAGCAACA
AGAAGTTCCTGCCATTCCAGCAGTTTGGCCGGGATATCGCCGATACCACAGACGC
CGTTAGAGATCCCCAGACACTGGAAATCCTGGACATCACCCCTTGCAGCTTCGGC
GGAGTGTCTGTGATCACCCCTGGCACCAACACCAGCAATCAGGTGGCAGTGCTG
TACCAGggcGTGAACTGTACCGAAGTGCCCGTGGCCATTCACGCCGATCAGCTGA
CACCTACATGGCGGGTGTACTCCACCGGCAGCAATGTGTTTCAGACCAGAGCCG
GCTGTCTGATCGGAGCCGAGtacGTGAACAATAGCTACGAGTGCGACATCCCCATC
GGCGCTGGCATCTGTGCCAGCTACCAGACACAGACAaagAGCcacGCCTCTGTGGC
CAGCCAGAGCATCATTGCCTACACAATGTCTCTGGGCGCCGAGAACAGCGTGGC
CTACTCCAACAACTCTATCGCTATCCCCACCAACTTCACCATCAGCGTGACCACA
GAGATCCTGCCTGTGTCCATGACCAAGACCAGCGTGGACTGCACCATGTACATCT
GCGGCGATTCCACCGAGTGCTCCAACCTGCTGCTGCAGTACGGCAGCTTCTGCAC
CCAGCTGaagAGAGCCCTGACAGGGATCGCCGTGGAACAGGACAAGAACACCCA
AGAGGTGTTCGCCCAAGTGAAGCAGATCTACAAGACCCCTCCTATCAAGtacTTCG
GCGGCTTCAATTTCAGCCAGATTCTGCCCGATCCTAGCAAGCCCAGCAAGCGGA
GCcctATCGAGGACCTGCTGTTCAACAAAGTGACACTGGCCGACGCCGGCTTCATC
AAGCAGTATGGCGATTGTCTGGGCGACATTGCCGCCAGGGATCTGATTTGCGCCC
AGAAGTTTaagGGACTGACAGTGCTGCCTCCTCTGCTGACCGATGAGATGATCGCC
CAGTACACATCTGCCCTGCTGGCCGGCACAATCACAAGCGGCTGGACATTTGGA
GCTGGCcctGCTCTGCAGATCCCCTTTccaATGCAGATGGCCTACCGGTTCAACGGC
ATCGGAGTGACCCAGAATGTGCTGTACGAGAACCAGAAGCTGATCGCCAACCAG
TTCAACAGCGCCATCGGCAAGATCCAGGACAGCCTGAGCAGCACAcccAGCGCCC
TGGGAAAGCTGCAGGACGTGGTCAACcacAATGCCCAGGCACTGAACACCCTGGT
CAAGCAGCTGTCCTCCaagTTCGGCGCCATCAGCTCTGTGCTGAACGATATCttcAG
CAGACTGGACccccctGAAGCCGAGGTGCAGATCGACAGACTGATCACCGGAAGGC
TGCAGTCCCTGCAGACCTACGTTACCCAGCAGCTGATCAGAGCCGCCGAGATTA
GAGCCTCTGCCAATCTGGCCGCCACCAAGATGTCTGAGTGTGTGCTGGGCCAGA
GCAAGAGAGTGGACTTTTGCGGCAAGGGCTACCACCTGATGAGCTTCCCTCAGTC
TGCCCCTCACGGCGTGGTGTTTCTGCACGTGACATACGTGCCCGCTCAAGAGAAG
AATTTCACCACCGCTCCAGCCATCTGCCACGACGGCAAAGCCCACTTTCCTAGAG
AAGGCGTGTTCGTGTCCAACGGCACCCATTGGTTCGTGACCCAGCGGAACTTCTA
CGAGCCCCAGATCATCACCACCGACAACACCTTCGTGTCTGGCAACTGCGACGTC
GTGATCGGCATTGTGAACAATACCGTGTACGACCCTCTGCAGCCCGAGCTGGAC
AGCTTCAAAGAGGAACTGGATAAGTACTTTAAGAACCACACAAGCCCCGACGTG
GACCTGGGCGATATCAGCGGAATCAATGCCAGCGTCGTGAACATCCAGAAAGAG
ATCGACCGGCTGAACGAGGTGGCCAAGAATCTGAACGAGAGCCTGATCGACCTG
CAAGAACTGGGGAAGTACGAGCAGTACATCAAGTGGCCC
Amino Acid Sequence of BA.1 S371 S375 (Signal peptide is in bold and underlined;
HXP-S ectodomain is underlined; S371 S375 is in bold, italics and underlined): SEQ ID
NO: 101
MFVFLVLLPLVSSQCVNLTTRTQLPPAYTNSFTRGVYYPDKVFRSSVLHSTQDLFLP
FFSNVTWFHVISGTNGTKRFDNPVLPFNDGVYFASIEKSNIIRGWIFGTTLDSKTQSLL
IVNNATNVVIKVCEFQFCNDPFLDHKNNKSWMESEFRVYSSANNCTFEYVSQPFLM
DLEGKQGNFKNLREFVFKNIDGYFKIYSKHTPIIVREPEDLPQGFSALEPLVDLPIGINI
TRFQTLLALHRSYLTPGDSSSGWTAGAAAYYVGYLQPRTFLLKYNENGTITDAVDC
ALDPLSETKCTLKSFTVEKGIYQTSNFRVQPTESIVRFPNITNLCPFDEVFNATRFASV
YAWNRKRISNCVADYSVLYNSAPFSTFKCYGVSPTKLNDLCFTNVYADSFVIRGDEV
RQIAPGQTGNIADYNYKLPDDFTGCVIAWNSNKLDSKVSGNYNYLYRLFRKSNLKPF
ERDISTEIYQAGNKPCNGVAGFNCYFPLRSYSFRPTYGVGHQPYRVVVLSFELLHAP
ATVCGPKKSTNLVKNKCVNFNFNGLKGTGVLTESNKKFLPFQQFGRDIADTTDAVR
DPQTLEILDITPCSFGGVSVITPGTNTSNQVAVLYQGVNCTEVPVAIHADQLTPTWRV
YSTGSNVFQTRAGCLIGAEYVNNSYECDIPIGAGICASYQTQTKSHASVASQSIIAYT
MSLGAENSVAYSNNSIAIPTNFTISVTTEILPVSMTKTSVDCTMYICGDSTECSNLLLQ
YGSFCTQLKRALTGIAVEQDKNTQEVFAQVKQIYKTPPIKYFGGFNFSQILPDPSKPS
KRSPIEDLLFNKVTLADAGFIKQYGDCLGDIAARDLICAQKFKGLTVLPPLLTDEMIA
QYTSALLAGTITSGWTFGAGPALQIPFPMQMAYRENGIGVTQNVLYENQKLIANQFN
SAIGKIQDSLSSTPSALGKLQDVVNHNAQALNTLVKQLSSKFGAISSVLNDIFSRLDPP
EAEVQIDRLITGRLQSLQTYVTQQLIRAAEIRASANLAATKMSECVLGQSKRVDFCG
KGYHLMSFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKEELDKYFKN
HTSPDVDLGDISGINASVVNIQKEIDRLNEVAKNLNESLIDLQELGKYEQYIKWP
Nucleotide Sequence of BA.1 S371 S375 (HXP-S ectodomain without signal peptide is
underlined; S371 S375 is in bold, italics and underlined): SEQ ID NO: 102
CAGTGTGTGAACCTGACCACAAGAACCCAGCTGCCTCCAGCCTACACCAACAGC
TTTACCAGAGGCGTGTACTACCCCGACAAGGTGTTCAGATCCAGCGTGCTGCACT
CTACCCAGGACCTGTTCCTGCCTTTCTTCAGCAACGTGACCTGGTTCCACgtgATCT
CCGGCACCAATGGCACCAAGAGATTCGACAACCCCGTGCTGCCCTTCAACGACG
GGGTGTACTTTGCCAGCatcGAGAAGTCCAACATCATCAGAGGCTGGATCTTCGGC
ACCACACTGGACAGCAAGACCCAGAGCCTGCTGATCGTGAACAACGCCACCAAC
GTGGTCATCAAAGTGTGCGAGTTCCAGTTCTGCAACGACCCCTTCCTGgacCACAA
GAACAACAAGAGCTGGATGGAAAGCGAGTTCCGGGTGTACAGCAGCGCCAACA
ACTGCACCTTCGAGTACGTGTCCCAGCCTTTCCTGATGGACCTGGAAGGCAAGCA
GGGCAACTTCAAGAACCTGCGCGAGTTCGTGTTCAAGAACATCGACGGCTACTTC
AAGATCTACAGCAAGCACACCCCTATCatcGTGCGGgaacccgaaGATCTGCCTCAGGG
CTTCTCTGCTCTGGAACCCCTGGTGGATCTGCCCATCGGCATCAACATCACCCGG
TTTCAGACACTGCTGGCCCTGCACAGAAGCTACCTGACACCTGGCGATAGCAGC
AGCGGATGGACAGCTGGTGCCGCCGCTTACTATGTGGGCTACCTGCAGCCTAGA
ACCTTTCTGCTGAAGTACAACGAGAACGGCACCATCACCGACGCCGTGGATTGT
GCTCTGGATCCTCTGAGCGAGACAAAGTGCACCCTGAAGTCCTTCACCGTGGAgA
AGGGCATCTACCAGACCAGCAACTTCCGGGTGCAGCCCACCGAATCCATCGTGC
GGTTCCCCAATATCACCAATCTGTGCCCCTTCgacGAGGTGTTCAATGCCACCAGA
TTCGCCTCTGTGTACGCCTGGAACCGGAAGCGGATCAGCAATTGCGTGGCCGACT
ACTCCGTGCTGTACAACtccGCCcctTTCagcACCTTCAAGTGCTACGGCGTGTCCCCT
ACCAAGCTGAACGACCTGTGCTTCACAAACGTGTACGCCGACAGCTTCGTGATCC
GGGGAGATGAAGTGCGGCAGATTGCCCCTGGACAGACAGGCaacATCGCCGACTA
CAACTACAAGCTGCCCGACGACTTCACCGGCTGTGTGATTGCCTGGAACAGCAA
CaagCTGGACTCCAAAGTCagcGGCAACTACAATTACCTGTACCGGCTGTTCCGGAA
GTCCAATCTGAAGCCCTTCGAGCGGGACATCTCCACCGAGATCTATCAGGCCGGC
aacaagCCTTGTAACGGCGTGgccGGCTTCAACTGCTACTTCCCACTGagaTCCTACagc
TTTagaCCCACAtacGGCGTGGGCcacCAGCCCTACAGAGTGGTGGTGCTGAGCTTCG
AACTGCTGCATGCCCCTGCCACAGTGTGCGGCCCTAAGAAgAGCACCAATCTCGT
GAAGAACAAATGCGTGAACTTCAACTTCAACGGCCTGaagGGCACCGGCGTGCTG
ACAGAGAGCAACAAGAAGTTCCTGCCATTCCAGCAGTTTGGCCGGGATATCGCC
GATACCACAGACGCCGTTAGAGATCCCCAGACACTGGAAATCCTGGACATCACC
CCTTGCAGCTTCGGCGGAGTGTCTGTGATCACCCCTGGCACCAACACCAGCAATC
AGGTGGCAGTGCTGTACCAGggcGTGAACTGTACCGAAGTGCCCGTGGCCATTCA
CGCCGATCAGCTGACACCTACATGGCGGGTGTACTCCACCGGCAGCAATGTGTTT
CAGACCAGAGCCGGCTGTCTGATCGGAGCCGAGtacGTGAACAATAGCTACGAGT
GCGACATCCCCATCGGCGCTGGCATCTGTGCCAGCTACCAGACACAGACAaagAG
CcacGCCTCTGTGGCCAGCCAGAGCATCATTGCCTACACAATGTCTCTGGGCGCCG
AGAACAGCGTGGCCTACTCCAACAACTCTATCGCTATCCCCACCAACTTCACCAT
CAGCGTGACCACAGAGATCCTGCCTGTGTCCATGACCAAGACCAGCGTGGACTG
CACCATGTACATCTGCGGCGATTCCACCGAGTGCTCCAACCTGCTGCTGCAGTAC
GGCAGCTTCTGCACCCAGCTGaagAGAGCCCTGACAGGGATCGCCGTGGAACAGG
ACAAGAACACCCAAGAGGTGTTCGCCCAAGTGAAGCAGATCTACAAGACCCCTC
CTATCAAGtacTTCGGCGGCTTCAATTTCAGCCAGATTCTGCCCGATCCTAGCAAG
CCCAGCAAGCGGAGCcctATCGAGGACCTGCTGTTCAACAAAGTGACACTGGCCG
ACGCCGGCTTCATCAAGCAGTATGGCGATTGTCTGGGCGACATTGCCGCCAGGG
ATCTGATTTGCGCCCAGAAGTTTaagGGACTGACAGTGCTGCCTCCTCTGCTGACC
GATGAGATGATCGCCCAGTACACATCTGCCCTGCTGGCCGGCACAATCACAAGC
GGCTGGACATTTGGAGCTGGCcctGCTCTGCAGATCCCCTTTccaATGCAGATGGCC
TACCGGTTCAACGGCATCGGAGTGACCCAGAATGTGCTGTACGAGAACCAGAAG
CTGATCGCCAACCAGTTCAACAGCGCCATCGGCAAGATCCAGGACAGCCTGAGC
AGCACAcccAGCGCCCTGGGAAAGCTGCAGGACGTGGTCAACcacAATGCCCAGGC
ACTGAACACCCTGGTCAAGCAGCTGTCCTCCaagTTCGGCGCCATCAGCTCTGTGC
TGAACGATATCttcAGCAGACTGGACccccctGAAGCCGAGGTGCAGATCGACAGACT
GATCACCGGAAGGCTGCAGTCCCTGCAGACCTACGTTACCCAGCAGCTGATCAG
AGCCGCCGAGATTAGAGCCTCTGCCAATCTGGCCGCCACCAAGATGTCTGAGTGT
GTGCTGGGCCAGAGCAAGAGAGTGGACTTTTGCGGCAAGGGCTACCACTGATG
AGCTTCCCTCAGTCTGCCCCTCACGGCGTGGTGTTTCTGCACGTGACATACGTGC
CCGCTCAAGAGAAGAATTTCACCACCGCTCCAGCCATCTGCCACGACGGCAAAG
CCCACTTTCCTAGAGAAGGCGTGTTCGTGTCCAACGGCACCCATTGGTTCGTGAC
CCAGCGGAACTTCTACGAGCCCCAGATCATCACCACCGACAACACCTTCGTGTCT
GGCAACTGCGACGTCGTGATCGGCATTGTGAACAATACCGTGTACGACCCTCTGC
AGCCCGAGCTGGACAGCTTCAAAGAGGAACTGGATAAGTACTTTAAGAACCACA
CAAGCCCCGACGTGGACCTGGGCGATATCAGCGGAATCAATGCCAGCGTCGTGA
ACATCCAGAAAGAGATCGACCGGCTGAACGAGGTGGCCAAGAATCTGAACGAG
AGCCTGATCGACCTGCAAGAACTGGGGAAGTACGAGCAGTACATCAAGTGGCCC
Amino Acid Sequence of BA.1 S371 S375 (HXP-S ectodomain without signal peptide is
underlined; S371 S375 is in bold, italics and underlined): SEQ ID NO: 103
QCVNLTTRTQLPPAYTNSFTRGVYYPDKVFRSSVLHSTQDLFLPFFSNVTWFHVISGT
NGTKRFDNPVLPFNDGVYFASIEKSNIIRGWIFGTTLDSKTQSLLIVNNATNVVIKVCE
FQFCNDPFLDHKNNKSWMESEFRVYSSANNCTFEYVSQPFLMDLEGKQGNFKNLRE
FVFKNIDGYFKIYSKHTPIIVREPEDLPQGFSALEPLVDLPIGINITRFQTLLALHRSYLT
PGDSSSGWTAGAAAYYVGYLQPRTFLLKYNENGTITDAVDCALDPLSETKCTLKSFT
VEKGIYQTSNFRVQPTESIVRFPNITNLCPFDEVFNATRFASVYAWNRKRISNCVADY
SVLYNSAPFSTFKCYGVSPTKLNDLCFTNVYADSFVIRGDEVRQIAPGQTGNIADYN
YKLPDDFTGCVIAWNSNKLDSKVSGNYNYLYRLFRKSNLKPFERDISTEIYQAGNKP
CNGVAGFNCYFPLRSYSFRPTYGVGHQPYRVVVLSFELLHAPATVCGPKKSTNLVK
NKCVNFNFNGLKGTGVLTESNKKFLPFQQFGRDIADTTDAVRDPQTLEILDITPCSFG
GVSVITPGTNTSNQVAVLYQGVNCTEVPVAIHADQLTPTWRVYSTGSNVFQTRAGC
LIGAEYVNNSYECDIPIGAGICASYQTQTKSHASVASQSIIAYTMSLGAENSVAYSNN
SIAIPTNFTISVTTEILPVSMTKTSVDCTMYICGDSTECSNLLLQYGSFCTQLKRALTGI
AVEQDKNTQEVFAQVKQIYKTPPIKYFGGFNFSQILPDPSKPSKRSPIEDLLFNKVTLA
DAGFIKQYGDCLGDIAARDLICAQKFKGLTVLPPLLTDEMIAQYTSALLAGTITSGWT
FGAGPALQIPFPMQMAYRENGIGVTQNVLYENQKLIANQFNSAIGKIQDSLSSTPSAL
GKLQDVVNHNAQALNTLVKQLSSKFGAISSVLNDIFSRLDPPEAEVQIDRLITGRLQS
LQTYVTQQLIRAAEIRASANLAATKMSECVLGQSKRVDFCGKGYHLMSFPQSAPHG
VVFLHVTYVPAQEKNFTTAPAICHDGKAHFPREGVFVSNGTHWFVTQRNFYEPQIIT
TDNTFVSGNCDVVIGIVNNTVYDPLQPELDSFKEELDKYFKNHTSPDVDLGDISGINA
SVVNIQKEIDRLNEVAKNLNESLIDLQELGKYEQYIKWP

Amino Acid Sequence of Wuhan strain (Signal peptide is in bold and
underlined; Spike protein ectodomain is underlined):
SEQ ID NO: 104
MFVFLVLLPLVSSQCVNLTTRTQLPPAYTNSFTRGVYYPDKVFRSSVLHSTQDLFLP
FFSNVTWFHAIHVSGTNGTKRFDNPVLPFNDGVYFASTEKSNIIRGWIFGTTLDSKTQ
SLLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLP
IGINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVFNATRF
ASVYAWNRKRISNCVADYSVLYNSASFSTFKCYGVSPTKLNDLCFTNVYADSFVIRG
DEVRQIAPGQTGKIADYNYKLPDDFTGCVIAWNSNNLDSKVGGNYNYLYRLFRKSN
LKPFERDISTEIYQAGSTPCNGVEGFNCYFPLQSYGFQPTNGVGYQPYRVVVLSFELL
HAPATVCGPKKSTNLVKNKCVNFNFNGLTGTGVLTESNKKFLPFQQFGRDIADTTD
AVRDPQTLEILDITPCSFGGVSVITPGTNTSNQVAVLYQDVNCTEVPVAIHADQLTPT
WRVYSTGSNVFQTRAGCLIGAEHVNNSYECDIPIGAGICASYQTQTNSPRRARSVAS
QSIIAYTMSLGAENSVAYSNNSIAIPTNFTISVTTEILPVSMTKTSVDCTMYICGDSTEC
SNLLLQYGSFCTQLNRALTGIAVEQDKNTQEVFAQVKQIYKTPPIKDFGGFNFSQILP
DPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRENGIGVTQNVLYENQKLI
ANQFNSAIGKIQDSLSSTASALGKLQDVVNQNAQALNTLVKQLSSNFGAISSVLNDIL
SRLDKVEAEVQIDRLITGRLQSLQTYVTQQLIRAAEIRASANLAATKMSECVLGQSK
RVDFCGKGYHLMSFPQSAPHGVVFLHVTYVPAQEKNFTTAPAICHDGKAHFPREGV
FVSNGTHWFVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQELGKYEQYI
KWP
Amino Acid Sequence of Wuhan strain (Spike protein ectodomain without
signal peptide is underlined):
SEQ ID NO: 105
QCVNLTTRTQLPPAYTNSFTRGVYYPDKVFRSSVLHSTQDLFLPFFSNVTWFHAIHVS
GTNGTKRFDNPVLPFNDGVYFASTEKSNIIRGWIFGTTLDSKTQSLLIVNNATNVVIK
VCEFQFCNDPFLGVYYHKNNKSWMESEFRVYSSANNCTFEYVSQPFLMDLEGKQG
NFKNLREFVFKNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASVYAWNRKRIS
NCVADYSVLYNSASFSTFKCYGVSPTKLNDLCFTNVYADSFVIRGDEVRQIAPGQTG
KIADYNYKLPDDFTGCVIAWNSNNLDSKVGGNYNYLYRLFRKSNLKPFERDISTEIY
QAGSTPCNGVEGFNCYFPLQSYGFQPTNGVGYQPYRVVVLSFELLHAPATVCGPKK
STNLVKNKCVNFNFNGLTGTGVLTESNKKFLPFQQFGRDIADTTDAVRDPQTLEILDI
TPCSFGGVSVITPGTNTSNQVAVLYQDVNCTEVPVAIHADQLTPTWRVYSTGSNVFQ
TRAGCLIGAEHVNNSYECDIPIGAGICASYQTQTNSPRRARSVASQSIIAYTMSLGAEN
SVAYSNNSIAIPTNFTISVTTEILPVSMTKTSVDCTMYICGDSTECSNLLLQYGSFCTQL
NRALTGIAVEQDKNTQEVFAQVKQIYKTPPIKDFGGFNFSQILPDPSKPSKRSFIEDLL
FNKVTLADAGFIKQYGDCLGDIAARDLICAQKFNGLTVLPPLLTDEMIAQYTSALLA
GTITSGWTFGAGAALQIPFAMQMAYRENGIGVTQNVLYENQKLIANQFNSAIGKIQD
SLSSTASALGKLQDVVNQNAQALNTLVKQLSSNFGAISSVLNDILSRLDKVEAEVQID
RLITGRLQSLQTYVTQQLIRAAEIRASANLAATKMSECVLGQSKRVDFCGKGYHLMS
FPQSAPHGVVFLHVTYVPAQEKNFTTAPAICHDGKAHFPREGVFVSNGTHWFVTQR
NFYEPQIITTDNTFVSGNCDVVIGIVNNTVYDPLQPELDSFKEELDKYFKNHTSPDVD
LGDISGINASVVNIQKEIDRLNEVAKNLNESLIDLQELGKYEQYIKWP

TABLE 4
Other Sequences

| | | |
|---|---|---|
| Linker Sequence | GGGGS | SEQ ID NO: 24 |
| SacII Restriction Sequence | CCGCGG | SEQ ID NO: 25 |
| NDV Gene End Sequence | TTAGAAAAAA | SEQ ID NO: 26 |
| NDV Gene Start Sequence | ACGGGTAGAA | SEQ ID NO: 27 |
| Kozak Sequence | CCGCCACC | SEQ ID NO: 28 |
| Signal Sequence | MFVFLVLLPLVSS | SEQ ID NO: 29 |

### 6. EXAMPLES

### 6.1 Example 1: Generation and Rescue of NDV-HXP-S

NDV-HXP-S expressing the spike protein of the Omicron variant were generated using the same approach as previously described (1-3). Briefly, Omicron BA. 1 sublineage specific mutations (A67V, HV69-70 deletion, T95I, G142D, VYY143-145 deletion, N211 deletion, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F) were introduced into the HXP-S backbone, which has ⁶⁸²RRAR⁶⁸⁵ changed to “A”, 6 Proline stabilizing mutations and the transmembrane domain (TM) and cytoplasmic tail (CT) from F protein (Table 5).

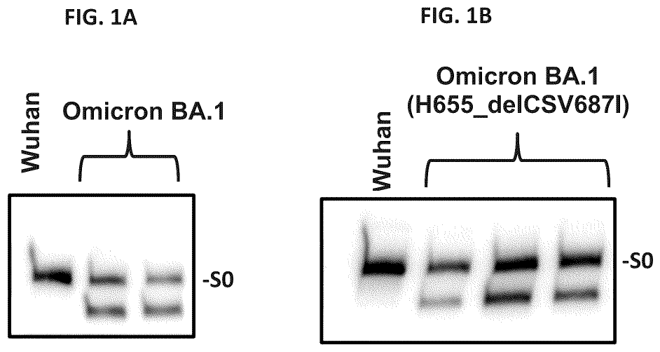

The viruses were rescued and passed once in eggs via limiting dilutions. The expression of the spike protein was examined by western blot (FIG. 1A). As compared to the Wuhan control, a degradation production below S0 was observed that could be identified by anti-S1 antibody. Based on previous structural work on SARS-COV spike with 2P (4), it is possible that such proteolysis event would not change the pre-fusion conformation due to the presence of 6 Proline stabilizing mutations. However, since it is not clear where the proteolysis occur and how it would affect the immunogenicity at this stage, different constructs were generated aiming to minimize the observed proteolysis event. One approach taken is to make more changes to the S1/S2 furin cleavage site. A previous study described a natural deletion and mutation at the furin cleavage site when the SARS-CoV-2 virus was passaged on Vero E6 cells (5). The changes include a larger portion of deletion at the furin cleavage site (⁶⁷⁹KSHRRARS⁶⁸⁶) and an adaptive V687I mutation (delCSV687I). It is also been reported that residues that are structurally close to the cleavage site might contribute to the S1/S2 cleavage such as H655Y, N679K and P681H/P (6). Therefore, the 655Y in the Omicron spike was changed back to the H655, and 679K and 681H have been deleted in the delCSV687I modifications. This construct was rescued and appeared to show some improvement to the S0 content (FIG. 1B). After assessing other candidates with a panel of different mutations, the proteolysis event might occur in the receptor binding domain (RBD) due to Omicron-specific mutations such as Q493R and Q498R which could introduce cleavage sites. Therefore, additional Omicron constructs with 493R and 498R changed back to Q493 and Q498 are being rescued and characterized (Table 5).

TABLE 5

| NDV-HXP-S Omicron constructs | Mutations |
|---|---|
| NDV-HXP-S Omicron BA.1 | A67V, HV69-70 deletion, T95I, G142D, |
| | VYY143-145 deletion, N211 deletion, L212I, |
| | ins214EPE, G339D, S371L, S373P, S375F, |
| | K417N, N440K, G446S, S477N, T478K, E484A, |
| | Q493R, G496S, Q498R, N501Y, Y505H, T547K, |
| | D614G, H655Y, N679K, P681H, N764K, D796Y, |
| | N856K, Q954H, N969K, L981F |
| NDV-HXP-S Omicron BA.1 | A67V, HV69-70 deletion, T95I, G142D, |
| (H655_delCSV687I) | VYY143-145 deletion, N211 deletion, L212I, |
| | ins214EPE, G339D, S371L, S373P, S375F, |
| | K417N, N440K, G446S, S477N, T478K, E484A, |
| | Q493R, G496S, Q498R, N501Y, Y505H, T547K, |
| | D614G, H655, NSPRRARS 679-686 deletion, |
| | V6871, N764K, D796Y, N856K, Q954H, N969K, |
| | L981F |
| NDV-HXP-S Omicron BA. 1 | A67V, HV69-70 deletion, T95I, G142D, |
| (Q493 Q498) | VYY143-145 deletion, N211 deletion, L212I, |
| | ins214EPE, G339D, S371L, S373P, S375F, |
| | K417N, N440K, G446S, S477N, T478K, E484A, |
| | Q493, G496S, Q498, N501Y, Y505H, T547K, |
| | D614G, H655Y, N679K, P681H, N764K, D796Y, |
| | N856K, Q954H, N969K, L981F |

### 6.1.1 REFERENCES CITED EXAMPLE 1

1. Sun W, McCroskery S, Liu W C, Leist S R, Liu Y, Albrecht R A, Slamanig S, Oliva J, Amanat F, Schafer A, Dinnon K H, 3rd, Innis B L, Garcia-Sastre A, Krammer F, Baric R S, Palese P. 2020. A Newcastle Disease Virus (NDV) Expressing a Membrane-Anchored Spike as a Cost-Effective Inactivated SARS-CoV-2 Vaccine. Vaccines (Basel) 8.
2. Sun W, Liu Y, Amanat F, Gonzalez-Dominguez I, McCroskery S, Slamanig S, Coughlan L, Rosado V, Lemus N, Jangra S, Rathnasinghe R, Schotsaert M, Martinez J L, Sano K, Mena I, Innis B L, Wirachwong P, Thai D H, Oliveira R D N, Scharf R, Hjorth R, Raghunandan R, Krammer F, Garcia-Sastre A, Palese P. 2021. A Newcastle disease virus expressing a stabilized spike protein of SARS-CoV-2 induces protective immune responses. Nat Commun 12:6197.
3. Sun W, Leist S R, McCroskery S, Liu Y, Slamanig S, Oliva J, Amanat F, Schaefer A, Dinnon K, Garcia-Sastre A, Krammer F, Baric R S, Palese P. 2020. Newcastle disease virus (NDV) expressing the spike protein of SARS-CoV-2 as vaccine candidate. bioRxiv doi: 10.1101/2020.07.26.221861.
4. Kirchdoerfer R N, Wang N, Pallesen J, Wrapp D, Turner H L, Cottrell C A, Corbett K S, Graham B S, Mclellan J S, Ward A B. 2018. Stabilized coronavirus spikes are resistant to conformational changes induced by receptor recognition or proteolysis. Sci Rep 8:15701.
5. Xia S, Lan Q, Su S, Wang X, Xu W, Liu Z, Zhu Y, Wang Q, Lu L, Jiang S. 2020. The role of furin cleavage site in SARS-CoV-2 spike protein-mediated membrane fusion in the presence or absence of trypsin. Signal Transduct Target Ther 5:92.
6 Escalera A, Gonzalez-Reiche A S, Aslam S, Mena I, Laporte M, Pearl R L, Fossati A, Rathnasinghe R, Alshammary H, van de Guchte A, Farrugia K, Qin Y, Bouhaddou M, Kehrer T, Zuliani-Alvarez L, Meekins D A, Balaraman V, McDowell C, Richt J A, Bajic G, Sordillo E M, Dejosez M, Zwaka T P, Krogan N J, Simon V, Albrecht R A, van Bakel H, Garcia-Sastre A, Aydillo T. 2022. Mutations in SARS-CoV-2 variants of concern link to increased spike cleavage and virus transmission. Cell Host Microbe 30:373-387 e7.

### 6.2 Example 2: Generation and Rescue of Additional NDV-HXP-S

NDV-HXP-S expressing the spike protein of the Omicron variant were generated using the same approach as described in previous work (1-3). Briefly, Omicron BA.1 sublineage specific mutations were introduced into the HXP-S backbone, which has ⁶⁸²RRAR⁶⁸⁵ changed to “A”, 6 Proline stabilizing mutations and the transmembrane domain (TM) and cytoplasmic tail (CT) from F protein (Table 6).

The viruses were rescued and passed once in eggs via limiting dilutions. The expression of the spike protein was examined by western blot (FIG. 2A). As compared to the Wuhan control, a cleavage product below S0 that could be identified by anti-S1 antibody. Based on previous structural work on SARS-COV spike with 2P (4), it is possible that such a proteolysis event would not change the pre-fusion conformation due to the presence of 6 Proline stabilizing mutations. However, since it is not clear where the proteolysis occurs and how it would affect the immunogenicity at this stage, different constructs were generated aiming to minimize the observed proteolysis event. One approach taken was to make more changes to the S1/S2 furin cleavage site. It has been reported that residues that are structurally close to the cleavage site might contribute to the S1/S2 cleavage such as H655Y, N679K and P681H/P (5). Therefore, single, double and triple mutations focusing on these residues in different combinations were made, which turned out to be unsuccessful to show an improvement (data not shown) (sequences of the constructs tested are shown in Table 7). A previous study described a natural deletion and mutation at the furin cleavage site when the SARS-CoV-2 virus was passaged on Vero E6 cells (6). The changes included a larger deletion at the furin cleavage site (⁶⁷⁹KSHRRARS⁶⁸⁶) and an adaptive V687I mutation (delCSV687I). Therefore, the 655Y in the Omicron spike was changed back to the H655 and 679K and 681H were deleted in the delCSV687I modifications (Table 6). This construct was rescued and appeared to show some improvement to the S0 content (likely due to structural changes) but did not completely prevent cleavage (FIG. 2B). After assessing candidates with a panel of different mutations, it was suspected that the proteolysis event might occur in the RBD domain due to Omicron-specific mutations. The attention of the inventors was drawn two sites containing more than one Omicron spike specific mutations. One site contains Q493R and Q498R, while the other site contains S371L, S373P and S375F. Single or double mutations were made regarding Q493R and Q498R, but no improvement was observed (sequences of the constructs tested are shown in Table 7). However, mutating amino acid residues 371, 373 and 375 back to S (Table 6) prevented the cleavage of the Omicron spike (FIG. 2C). Therefore, it was concluded that one, two or three of amino acid residue S371L, S373P and S375F cause the cleavage of the spike protein.

TABLE 6
NDV-HXP-S Omicron Constructs

| NDV-HXP-S Omicron | |
|---|---|
| Constructs | Mutations |
| NDV-HXP-S Omicron | A67V, HV69-70 deletion, T95I, G142D, VYY143-145 deletion, |
| BA.1 | N211 deletion, L212I, ins214EPE, G339D, S371L, S373P, |
| | S375F, K417N, N440K, G446S, S477N, T478K, E484A, Q493R, |
| | G496S, Q498R, N501Y, Y505H, T547K, D614G, H655Y, |
| | N679K, P681H, N764K, D796Y, N856K, Q954H, N969K, |
| | L981F |
| NDV-HXP-S Omicron | A67V, HV69-70 deletion, T95I, G142D, VYY143-145 deletion, |
| BA.1 | N211 deletion, L212I, ins214EPE, G339D, S371L, S373P, |
| (H655_delCSV687I) | S375F, K417N, N440K, G446S, S477N, T478K, E484A, Q493R, |
| | G496S, Q498R, N501Y, Y505H, T547K, D614G, H655, |
| | NSPRRARS 679-686 deletion, V6871, N764K, D796Y, N856K, |
| | Q954H, N969K, L981F |
| NDV-HXP-S Omicron | A67V, HV69-70 deletion, T95I, G142D, VYY143-145 deletion, |
| BA.1 (S371 S373 S375) | N211 deletion, L212I, ins214EPE, G339D, S371, S373, S375, |
| | K417N, N440K, G446S, S477N, T478K, E484A, Q493R, G496S, |
| | Q498R, N501Y, Y505H, T547K, D614G, H655Y, N679K, |
| | P681H, N764K, D796Y, N856K, Q954H, N969K, L981F |

TABLE 7
Examples of other mutant constructs that are still cleaved
to a level similar to that of the NDV-HXP-S Omicron BA.1.

| NDV-HXP-S Omicron | |
|---|---|
| constructs | Mutations |
| NDV-HXP-S Omicron | A67V, HV69-70 deletion, T95I, G142D, VYY143-145 deletion, |
| BA.1 (N679) | N211 deletion, L212I, ins214EPE, G339D, S371L, S373P, |
| | S375F, K417N, N440K, G446S, S477N, T478K, E484A, Q493R, |
| | G496S, Q498R, N501Y, Y505H, T547K, D614G, H655Y, N679, |
| | P681H, N764K, D796Y, N856K, Q954H, N969K, L981F |
| NDV-HXP-S Omicron | A67V, HV69-70 deletion, T95I, G142D, VYY143-145 deletion, |
| BA.1 (P681 N764) | N211 deletion, L212I, ins214EPE, G339D, S371L, S373P, |
| | S375F, K417N, N440K, G446S, S477N, T478K, E484A, Q493R, |
| | G496S, Q498R, N501Y, Y505H, T547K, D614G, H655Y, |
| | N679K, P681, N764, D796Y, N856K, Q954H, N969K, L981F |
| NDV-HXP-S Omicron | A67V, HV69-70 deletion, T95I, G142D, VYY143-145 deletion, |
| BA.1 (N679K P681 | N211 deletion, L212I, ins214EPE, G339D, S371L, S373P, |
| N764) | S375F, K417N, N440K, G446S, S477N, T478K, E484A, Q493R, |
| | G496S, Q498R, N501Y, Y505H, T547K, D614G, H655Y, N679, |
| | P681, N764, D796Y, N856K, Q954H, N969K, L981F |
| NDV-HXP-S Omicron | A67V, HV69-70 deletion, T95I, G142D, VYY143-145 deletion, |
| BA.1 | N211 deletion, L212I, ins214EPE, G339D, S371L, S373P, |
| (H655) | S375F, K417N, N440K, G446S, S477N, T478K, E484A, Q493R, |
| | G496S, Q498R, N501Y, Y505H, T547K, D614G, H655, N679K, |
| | P681H, N764K, D796Y, N856K, Q954H, N969K, L981F |
| NDV-HXP-S Omicron | A67V, HV69-70 deletion, T95I, G142D, VYY143-145 deletion, |
| BA.1 | N211 deletion, L212I, ins214EPE, G339D, S371L, S373P, |
| (P681) | S375F, K417N, N440K, G446S, S477N, T478K, E484A, Q493R, |
| | G496S, Q498R, N501Y, Y505H, T547K, D614G, H655Y, |
| | N679K, P681, N764K, D796Y, N856K, Q954H, N969K, L981F |
| NDV-HXP-S Omicron | A67V, HV69-70 deletion, T95I, G142D, VYY143-145 deletion, |
| BA.1 | N211 deletion, L212I, ins214EPE, G339D, S371L, S373P, |
| (N679 P681) | S375F, K417N, N440K, G446S, S477N, T478K, E484A, Q493R, |
| | G496S, Q498R, N501Y, Y505H, T547K, D614G, H655Y, N679, |
| | P681, N764K, D796Y, N856K, Q954H, N969K, L981F |
| NDV-HXP-S Omicron | A67V, HV69-70 deletion, T95I, G142D, VYY143-145 deletion, |
| BA.1 | N211 deletion, L212I, ins214EPE, G339D, S371L, S373P, |
| (H655 P681) | S375F, K417N, N440K, G446S, S477N, T478K, E484A, Q493R, |
| | G496S, Q498R, N501Y, Y505H, T547K, D614G, H655, N679K, |
| | P681, N764K, D796Y, N856K, Q954H, N969K, L981F |
| NDV-HXP-S Omicron | A67V, HV69-70 deletion, T95I, G142D, VYY143-145 deletion, |
| BA.1 | N211 deletion, L212I, ins214EPE, G339D, S371L, S373P, |
| (H655 N679 P681) | S375F, K417N, N440K, G446S, S477N, T478K, E484A, Q493R, |
| | G496S, Q498R, N501Y, Y505H, T547K, D614G, H655, N679, |
| | P681, N764K, D796Y, N856K, Q954H, N969K, L981F |
| NDV-HXP-S Omicron | A67V, HV69-70 deletion, T95I, G142D, VYY143-145 deletion, |
| BA.1 | N211 deletion, L212I, ins214EPE, G339D, S371L, S373P, |
| (Q493) | S375F, K417N, N440K, G446S, S477N, T478K, E484A, Q493, |
| | G496S, Q498R, N501Y, Y505H, T547K, D614G, H655Y, |
| | N679K, P681H, N764K, D796Y, N856K, Q954H, N969K, |
| | L981F |
| NDV-HXP-S Omicron | A67V, HV69-70 deletion, T95I, G142D, VYY143-145 deletion, |
| BA.1 | N211 deletion, L212I, ins214EPE, G339D, S371L, S373P, |
| (Q498) | S375F, K417N, N440K, G446S, S477N, T478K, E484A, Q493R, |
| | G496S, Q498, N501Y, Y505H, T547K, D614G, H655Y, N679K, |
| | P681H, N764K, D796Y, N856K, Q954H, N969K, L981F |
| NDV-HXP-S Omicron | A67V, HV69-70 deletion, T95I, G142D, VYY143-145 deletion, |
| BA.1 | N211 deletion, L212I, ins214EPE, G339D, S371L, S373P, |
| (Q493 Q498) | S375F, K417N, N440K, G446S, S477N, T478K, E484A, Q493, |
| | G496S, Q498, N501Y, Y505H, T547K, D614G, H655Y, N679K, |
| | P681H, N764K, D796Y, N856K, Q954H, N969K, L981F |

### 6.2.1 REFERENCES CITED IN EXAMPLE 2

1. Sun W, McCroskery S, Liu W C, Leist S R, Liu Y, Albrecht R A, Slamanig S, Oliva J, Amanat F, Schafer A, Dinnon K H, 3rd, Innis B L, Garcia-Sastre A, Krammer F, Baric R S, Palese P. 2020. A Newcastle Disease Virus (NDV) Expressing a Membrane-Anchored Spike as a Cost-Effective Inactivated SARS-CoV-2 Vaccine. Vaccines (Basel) 8.
2. Sun W, Liu Y, Amanat F, Gonzalez-Dominguez I, McCroskery S, Slamanig S, Coughlan L, Rosado V, Lemus N, Jangra S, Rathnasinghe R, Schotsaert M, Martinez J L, Sano K, Mena I, Innis B L, Wirachwong P, Thai D H, Oliveira R D N, Scharf R, Hjorth R, Raghunandan R, Krammer F, Garcia-Sastre A, Palese P. 2021. A Newcastle disease virus expressing a stabilized spike protein of SARS-CoV-2 induces protective immune responses. Nat Commun 12:6197.
3. Sun W, Leist S R, McCroskery S, Liu Y, Slamanig S, Oliva J, Amanat F, Schaefer A, Dinnon K, Garcia-Sastre A, Krammer F, Baric R S, Palese P. 2020. Newcastle disease virus (NDV) expressing the spike protein of SARS-CoV-2 as vaccine candidate. bioRxiv doi: 10.1101/2020.07.26.221861.
4. Kirchdoerfer R N, Wang N, Pallesen J, Wrapp D, Turner H L, Cottrell C A, Corbett K S, Graham B S, McLellan J S, Ward A B. 2018. Stabilized coronavirus spikes are resistant to conformational changes induced by receptor recognition or proteolysis. Sci Rep 8:15701.
5 Escalera A, Gonzalez-Reiche A S, Aslam S, Mena I, Laporte M, Pearl R L, Fossati A, Rathnasinghe R, Alshammary H, van de Guchte A, Farrugia K, Qin Y, Bouhaddou M, Kehrer T, Zuliani-Alvarez L, Meekins D A, Balaraman V, McDowell C, Richt J A, Bajic G, Sordillo E M, Dejosez M, Zwaka T P, Krogan N J, Simon V, Albrecht R A, van Bakel H, Garcia-Sastre A, Aydillo T. 2022. Mutations in SARS-CoV-2 variants of concern link to increased spike cleavage and virus transmission. Cell Host Microbe 30:373-387 e7.
6 Xia S, Lan Q, Su S, Wang X, Xu W, Liu Z, Zhu Y, Wang Q, Lu L, Jiang S. 2020. The role of furin cleavage site in SARS-CoV-2 spike protein-mediated membrane fusion in the presence or absence of trypsin. Signal Transduct Target Ther 5:92.

### 6.3 Example 3: Generation and Rescue of Additional NDV-HXP-S

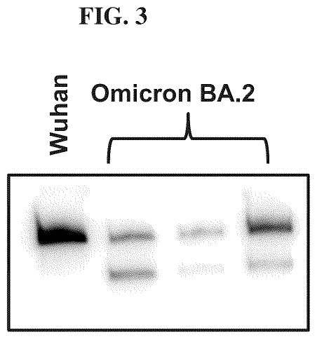

With increased prevalence of BA.2 and BA.4/5 during this time (Table 8), NDV-HXP-S expressing the BA.2 spike protein was generated. Omicron BA.2 sublineage specific mutations were introduced into the HXP-S backbone, which has ⁶⁸²RRAR⁶⁸⁵ changed to “A”, 6 Proline stabilizing mutations and the transmembrane domain (TM) and cytoplasmic tail (CT) from F protein. The virus was rescued and passed once in eggs via limiting dilutions. The expression of the spike protein was examined by western blot (FIG. 3). As compared to the Wuhan control, a cleavage product below S0 that could be identified by anti-S1 antibody. The BA.2 and BA4/5 spike protein both contain S371F, S373P and S375F mutations (see NDV-HXP-S Omicron BA.2 and NDV-HXP-S Omicron BA.4/5 in Table 8). Omicron BA.2 sublineage specific mutations are introduced into the HXP-S backbone, which has ⁶⁸²RRAR⁶⁸⁵ changed to “A”, 6 Proline stabilizing mutations, the transmembrane domain (TM) and cytoplasmic tail (CT) from F protein, and serines are maintained at amino acid positions 371, 373 and 375. (See NDV-HXP-S Omicron BA.2 (S371 S373 S375) in Table 8.) Similarly, Omicron BA.4/5 sublineage specific mutations are introduced into the HXP-S backbone, which has ⁶⁸²RRAR⁶⁸⁵ changed to “A”, 6 Proline stabilizing mutations, the transmembrane domain (TM) and cytoplasmic tail (CT) from F protein, and serines are maintained at amino acid positions 371, 373 and 375. (See NDV-HXP-S Omicron BA.4/5 (S371 S373 S375) in Table 8.)

TABLE 8

| NDV-HXP-S Omicron | |
|---|---|
| Constructs | Mutations |
| NDV-HXP-S Omicron | T19I, del24-26(LPP), A27S, G142D, V213G, G339D, S371F, |
| BA.2 | S373P, S375F. T376A, D405N, R408S, K417N, N440K, S477N, |
| | T478K, E484A, Q493R, Q498R, N501Y, Y505H, D614G, H655Y, |
| | N679K, P681H, N764K, D796Y, Q954H, N969K |
| NDV-HXP-S Omicron | T19I, del24-26(LPP),A27S, del69-70(HV), G142D, V213G, |
| BA.4/5 | G339D, S371F, S373P, S375F, T376A, D405N, R408S, K417N, |
| | N440K, S477N, L452R, T478K, E484A, F486V, Q498R, N501Y, |
| | Y505H, D614G, H655Y, N679K, P681H, N764K, D796Y, Q954H, |
| | N969K |
| NDV-HXP-S Omicron | T19I, del24-26(LPP), A27S, G142D, V213G, G339D, S371, S373, |
| BA.2 | S375, T376A, D405N, R408S, K417N, N440K, S477N, T478K, |
| (S371 S373 S375) | E484A, Q493R, Q498R, N501Y, Y505H, D614G, H655Y, N679K, |
| | P681H, N764K, D796Y, Q954H, N969K |
| NDV-HXP-S Omicron | T19I, del24-26(LPP),A27S, del69-70(HV), G142D, V213G, |
| BA.4/5 | G339D, S371, S373, S375, T376A, D405N, R408S, K417N, |
| (S371 S373 S375) | N440K, S477N, L452R, T478K, E484A, F486V, Q498R, N501Y, |
| | Y505H, D614G, H655Y, N679K, P681H, N764K, D796Y, Q954H, |
| | N969K |

### 6.4 Example 4: Generation and Rescue of Additional NDV-HXP-S

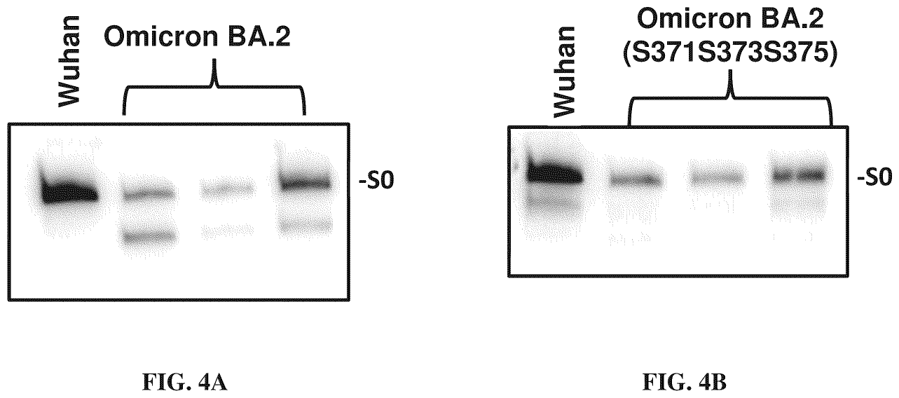

As previously disclosed in Example 2, S371 S373 and S375 in the BA.1 spike could stabilize the spike protein in its uncleaved form. It was hypothesized that the same amino acid composition at 371, 373, and 375 could also stabilize the BA.2 spike since the BA.2 wild-type spike (NDV-HXP-S Omicron BA.2) was observed to be cleaved when expressed by NDV vector (FIG. 4A). Indeed, when amino acid residues 371, 373 and 375 were reverted back to S371, S373 and S375 in the BA.2 SSS (NDV-HXP-S Omicron BA.2 SSS), the spike protein was stabilized (FIG. 4B). See Table 9 for the amino acid substitutions of BA.2 sequences relative to the ancestral HXP-S.

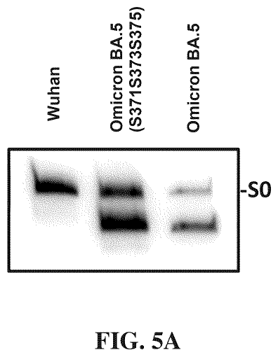

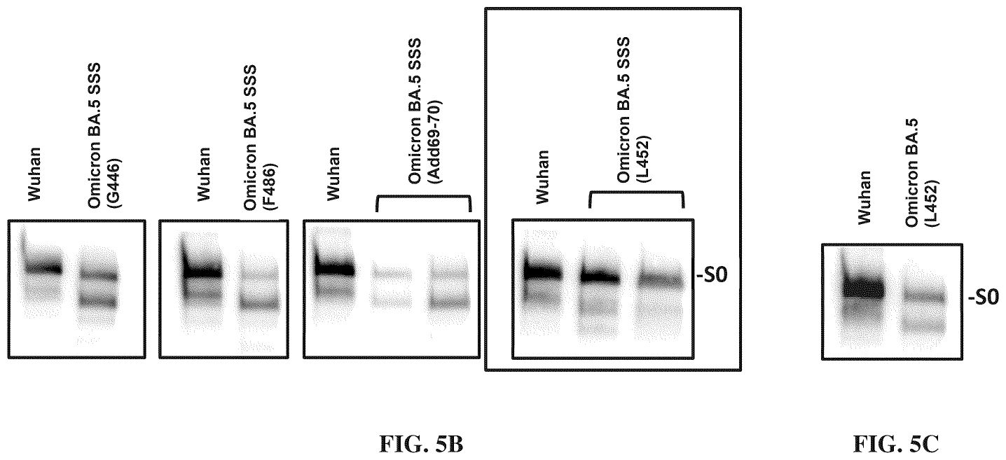

To develop a vaccine candidates for the BA.5, corresponding amino acid substitutions showed in Table 9 were added. Of note, G446S was added to represent an essential mutation in the BA.2.75.2 variant of concern (VOC) that was circulating at that time, which was also present in BA.1. As expected, the BA.5 spike was cleaved. Subsequently, S371, S373 and S375 were applied to the BA.5 construct, but surprisingly, they were not found sufficient to prevent cleavage of the spike (FIG. 5A). To determine which amino acid substitution(s) contributed to the cleavage of the spike in the presence of the SSS, four (4) essential changes from BA.2 were highlighted, including del69-70, G446S, L452R and F486V. Each substitution was reverted back individually to ancestral sequence designated as Add69-70, G446, L452 and F486 in the presence of SSS. It was found that L452R, but not del69-70, G446S, F486V, causes the cleavage of the spike as L452 stabilized the spike protein (FIG. 5B). BA.5 without the SSS but with L452 was still cleaved indicating both were needed to stabilize the spike proteins (FIGS. 5B and 5C). Therefore, BA.5 SSS L452 was pursued as the BA.5-like vaccine candidate.

TABLE 9
Amino acid substitutions of BA.2 and BA.5
sequences relative to the ancestral HXP-S

| NDV-HXP-S Omicron | |
|---|---|
| constructs | Mutations |
| NDV-HXP-S Omicron | T19I, del24-26(LPP), A27S, G142D, V213G, G339D, S371F, |
| BA.2 | S373P, S375F, T376A, D405N, R408S, K417N, N440K, |
| | S477N, T478K, E484A, Q493R, Q498R, N501Y, Y505H, |
| | D614G, H655Y, N679K, P681H, N764K, D796Y, Q954H, |
| | N969K |
| NDV-HXP-S Omicron | T19I, del24-26(LPP), A27S, G142D, V213G, G339D, S371, |
| BA.2 SSS | S373, S375, T376A, D405N, R408S, K417N, N440K, S477N, |
| | T478K, E484A, Q493R, Q498R, N501Y, Y505H, D614G, |
| | H655Y, N679K, P681H, N764K, D796Y, Q954H, N969K |
| NDV-HXP-S Omicron | T19I, del24-26(LPP),A27S, del69-70(HV), G142D, V213G, |
| BA.5 | G339D, S371F, S373P, S375F, T376A, D405N, R408S, |
| | K417N, N440K, *G446S, S477N, L452R, T478K, E484A, |
| | F486V, Q498R, N501Y, Y505H, D614G, H655Y, N679K, |
| | P681H, N764K, D796Y, Q954H, N969K |
| NDV-HXP-S Omicron | T191, del24-26(LPP),A27S, del69-70(HV), G142D, V213G, |
| BA.5 SSS L452 | G339D, S371, S373, S375, T376A, D405N, R408S, K417N, |
| | N440K, *G446S, S477N, L452, T478K, E484A, F486V, |
| | Q498R, N501Y, Y505H, D614G, H655Y, N679K, P681H, |
| | N764K, D796Y, Q954H, N969K |

Note:
*G446S was added for breadth since BA.2.75.2 was circulating as a variant of concern at the same time and G446S was one of the key mutations, which was also present in BA.1.

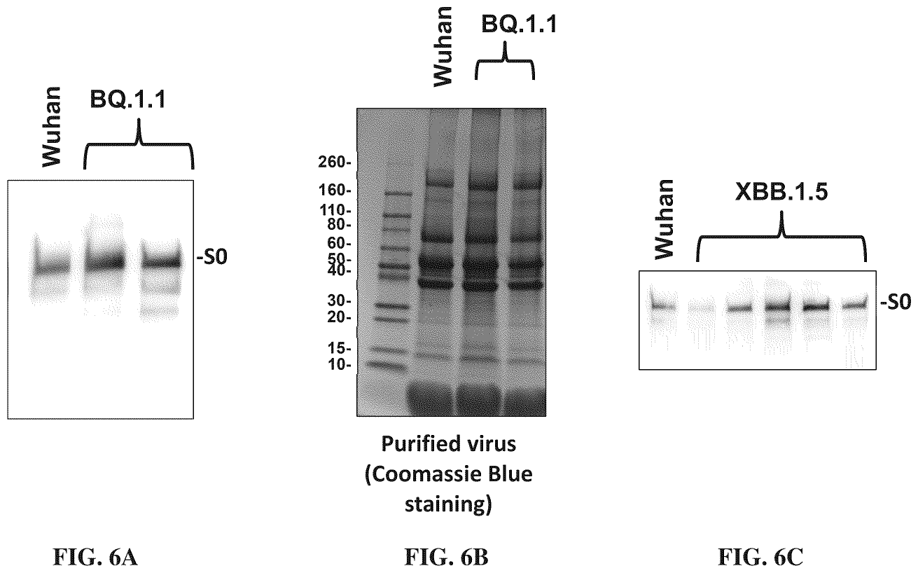

Additionally, NDV vectors expressing the BQ.1.1 variant spike protein or the XBB.1.5 variant spike protein were generated with corresponding amino acid substitutions based on the ancestral HXP-S expressed by the NDV (Table 10). Both BQ.1.1 (FIG. 6A) and XBB.1.5 (FIG. 6C) showed limited cleavage in the spike, which was likely influenced by novel strain-specific mutations in the receptor binding domain (RBD), that the cleavage sites were masked or eliminated. As sufficient uncleaved S0 was expressed (e.g., purified BQ.1.1 as shown in FIG. 6B), no additional modifications were needed for BQ.1.1 and XBB.1.5.

TABLE 10
Amino acid substitutions of BQ.1.1 and XBB.1.5 relative to the ancestral HXP-S

| NDV-HXP-S Omicron | |
|---|---|
| constructs | mutations |
| NDV-HXP-S Omicron | T19I, del24-26(LPP), A27S, del69-70, G142D, V213G, |
| BQ.1.1 | G339D,R346T, S371F, S373P, S375F, T376A, D405N, R408S, |
| | K417N, N440K, K444T, L452R, N460K, S477N, T478K, |
| | E484A, F486V, Q498R, N501Y, Y505H, D614G, H655Y, |
| | N679K, P681H, N764K, D796Y, Q954H, N969K |
| NDV-HXP-S Omicron | T19I, del24-26(LPP), A27S, V83A, G142D, del144, H146Q, |
| XBB.1.5 | Q183E, V213E, G252V, G339H, R346T, L368I, S371F, S373P, |
| | S375F, T376A, D405N, R408S, K417N, N440K, V445P, |
| | G446S, N460K, S477N, T478K, E484A, F486P, F490S, |
| | Q498R, N501Y, Y505H, D614G, H655Y, N679K, P681H, |
| | N764K, D796Y, Q954H, N969K |

### 6.5 Example 5: Immunization in Mice with NDV-HXP-S Vaccines

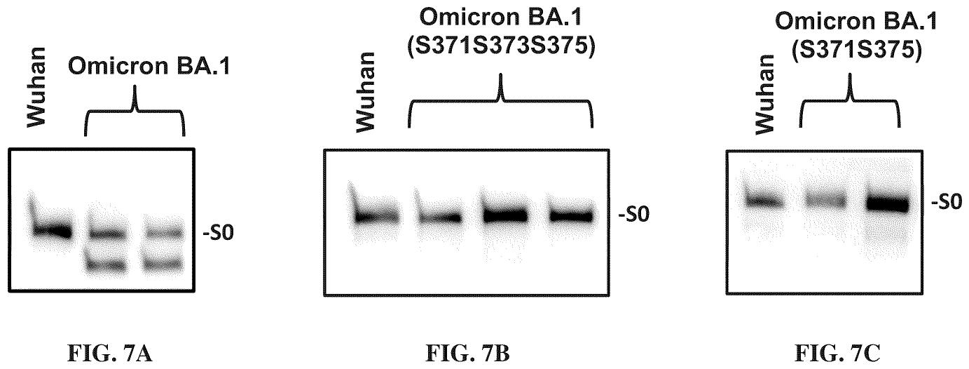

As previously identified in Example 2, changing amino acid residues of 371, 373 and 375 in the Omicron BA.1 spike protein back to ancestral S371 S373 and S375 (SSS) could stabilize the spike protein expressed by NDV (FIGS. 7A and 7B). In the process of characterizing how many mutations out of the three were necessary, S371 and S375 were observed to be sufficient to stabilize the BA. 1 spike without affecting the expression level of the protein (FIG. 7C). See Table 11 for mutations in NDV-HXP-S Omicron BA.1 (S371, S375).

TABLE 11
NDV-HXP-S Omicron Construct

| NDV-HXP-S Omicron | |
|---|---|
| Construct | Mutations |
| NDV-HXP-S Omicron | A67V, HV69-70 deletion, T95I, G142D, VYY143-145 |
| BA.1 (S371, S375) | deletion, N211 deletion, L212I, ins214EPE, G339D, S371, |
| | S373P, S375, K417N, N440K, G446S, S477N, T478K, E484A, |
| | Q493R, G496S, Q498R, N501Y, Y505H, T547K, D614G, |
| | H655Y, N679K, P681H, N764K, D796Y, N856K, Q954H, |
| | N969K, L981F |

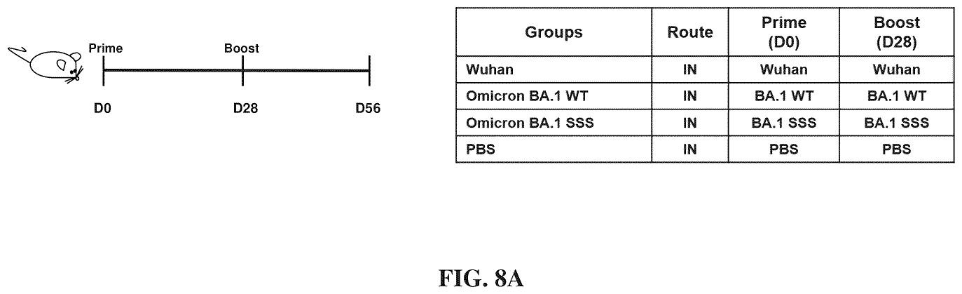

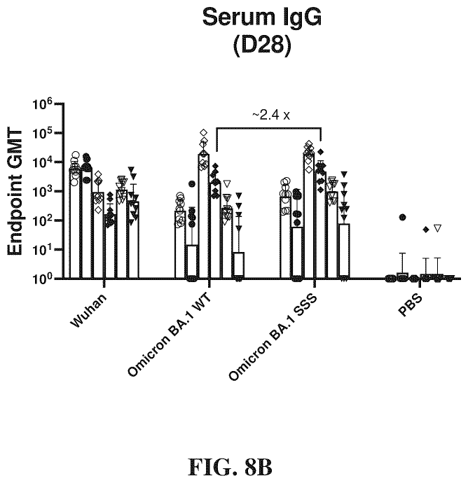

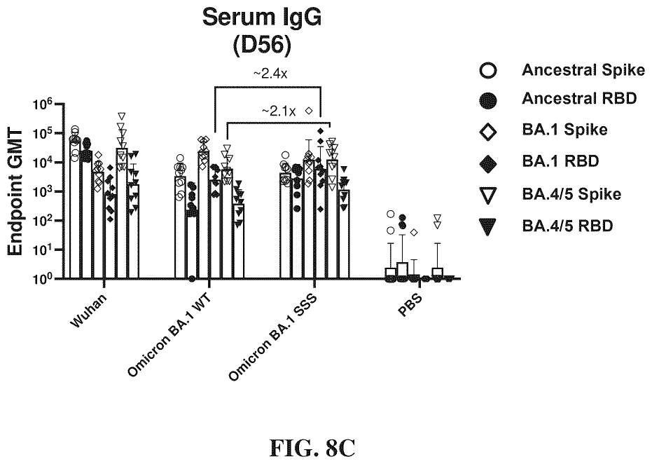

To compare the immunogenicity of the cleaved BA. 1 wild-type spike (NDV-HXP-S Omicron BA.1) and the stabilized BA.1 SSS spike (NDV-HXP-S Omicron BA.1 (S371, S373, S375). An immunization study in mice testing live vaccine via the intranasal route was performed. Briefly, female BALB/c mice were immunized intranasally with 106 EID50 of NDV-HXP-S (Wuhan), NDV-HXP-S (BA.1 WT), and NDV-HXP-S (BA.1 SSS) twice with a 4-week interval. Four weeks after the first and second dose, serum IgG against ancestral spike protein, ancestral RBD protein, BA. 1 spike protein, BA. 1 RBD protein, BA.4/5 spike protein, and BA.4/5 RBD protein were measured by ELISAs (FIG. 8A). After the first vaccination, mice that were vaccinated with the Wuhan vaccine developed high levels of ancestral spike and RBD-binding antibodies, which cross-reacted to BA. 1 and BA.4/5 spike and RBD to a much lower level. Mice that received either BA. 1 WT and BA.1 SSS developed high levels of BA. 1 spike and RBD binding antibodies, substantially higher than those induced by the Wuhan vaccine. With the BA. 1 spike-binding antibodies developed to a similar level of the two groups, the BA.1 SSS induced ˜2.4 fold BA.1 RBD-binding antibodies of that induced by the BA.1 WT vaccines. The difference of binding antibodies to the ancestral and BA.4/5 antigens was more pronounced, that BA. 1 SSS appeared to induce more cross-reactive antibodies than the BA. 1 WT vaccine (FIG. 8B). At four weeks after the booster dose, the Wuhan vaccine induced more binding antibodies to all three types of antigens overall following a similar binding profile as that of post-prime vaccination. Again, the second booster dose of BA. 1 WT and BA.1 SSS induced similar levels of BA.1 spike-binding antibodies, but the difference in BA.1 RBD-binding was still about 2.4 fold. It was also clear that BA.1 SSS vaccine induced substantially more ancestral and BA.4/5 RBD-binding antibodies. Even for cross-reactive antibodies to the BA.4/5 spike, the difference was more than 2 fold (FIG. 8C). These data indicate that as compared to the unstable BA.1 WT spike, the stabilized BA.1 SSS induced better strain-specific as well as cross-reactive antibody responses in vivo in naïve animals.

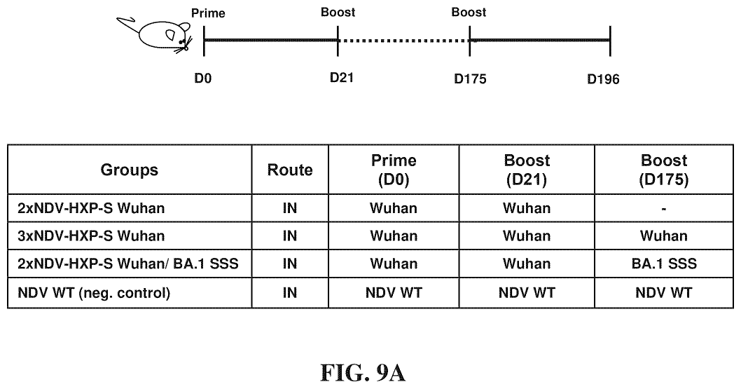

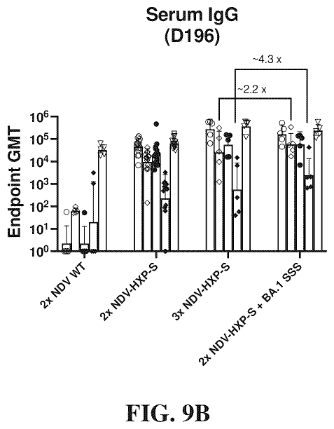

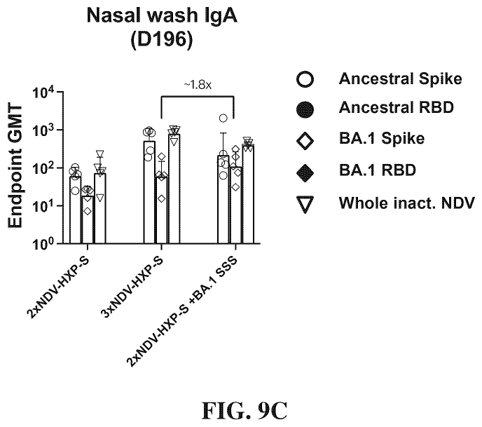

To investigate humoral responses using NDV-HXP-S ancestral and BA.1 SSS as a booster vaccine, a three-vaccination series study was first performed in naïve female BALB/c mice with NDV-based vaccines. Specifically, mice were vaccinated with NDV-HXP-S Wuhan strain twice with a 3-week interval between the first and second dose. Approximately 5 months later, a third booster with either the same ancestral NDV-HXP-S Wuhan vaccine or the NDV-HXP-S BA.1 SSS vaccine was given. This was essentially to mimic a primary vaccination series with NDV-based vaccine and use the NDV-based vaccine again as a third booster when antibodies waned, similar to the real-world COVID-19 booster strategy. Each vaccination was administered intranasally at the same dose (106 EID50) to each mouse. Antibodies induced by the following vaccinations were measured: two vaccinations of the NDV-HXP-S Wuhan (2×NDV-HXP-S), three vaccinations of NDV-HXP-S Wuhan (3×NDV-HXP-S), two vaccinations of NDV-HXP-S Wuhan followed by NDV-HXP-S BA.1 SSS booster, and two vaccinations of the vector (2×NDV WT) (FIG. 9A). Serum IgG titers were measured for all four conditions against the Wuhan spike, BA. 1 spike, Wuhan RBD, BA.1 RBD as well as the vector (inactivated whole virion of NDV WT) by ELISAs. It was observed that as compared to the 2×NDV-HXP-S Wuhan group, a third booster of NDV-HXP-S Wuhan was able to further increase antibody responses to all protein substrates (Wuhan S, BA.1 S, Wuhan RBD, BA.1 RBD) and the vector. Interestingly, as the NDV-HXP-S BA.1 SSS booster brought antibodies against the ancestral and BA. 1 spikes to a similar level as the ancestral booster, it induced more binding-antibodies towards both the ancestral RBD as well as the BA. 1 RBD (FIG. 9B). In the nasal wash, a booster effect of the third dose was also observed at increasing Wuhan spike-, BA.1 spike- and vector-targeting IgA titers, that BA.1 SSS vaccine appeared to increase the BA.1 spike binding antibodies more substantially than the ancestral vaccine (FIG. 9C). In conclusion, a third intransal (i.n.) booster of NDV-HXP-S can increase antibodies titers after a primary i.n. vaccination series with the same vector-based vaccines 5 month later in the mouse model at 106 EID50 per mouse. In this particular study, NDV-HXP-S BA. 1 SSS booster induced more BA. 1 strain specific antibodies than the Wuhan vaccine.

### 7. EMBODIMENTS

The following are exemplary embodiments:

1. A recombinant protein comprising a derivative of a SARS-CoV-2 Omicron spike protein ectodomain, wherein the derivative comprises the ectodomain of the amino acid sequence of SEQ ID NO: 104 without the signal peptide and with amino acid modifications, wherein the amino acid modifications comprise: (1) an amino acid substitution at amino acid residues corresponding to amino acid residues 682 to 685 (RRAR) of SEQ ID NO: 104 to a single alanine; (2) amino acid substitutions at amino acid residues corresponding to the following amino acid residues of SEQ ID NO: 104: F817P, A892P, A899P, A942P, K986P, and V987P; and (3) two or more amino acid modifications to the amino acid sequence of the ectodomain of SEQ ID NO:104 to amino acid residues found at the corresponding amino acid positions in the Omicron spike protein ectodomain, wherein the two or more amino acid modifications comprise two or more amino acid modifications at amino acid positions corresponding to the indicated amino acid positions of SEQ ID NO: 104: N440K, S477N, Y505H, N679K, N764K, D796Y, Q954H, and/or N969K.

2. The recombinant protein of embodiment 1, wherein the two or more amino acid modifications comprise the following amino acid modifications at amino acid positions corresponding to the indicated amino acid positions of SEQ ID NO: 104: N440K, S477N, Y505H, N679K, N764K, D796Y, Q954H, and N969K.

3. The recombinant protein of any one of embodiments 1 or 2, wherein the two or more amino acid modifications does not include amino acid modifications at amino acid positions corresponding to amino acid positions of 371 and 375 in SEQ ID NO:104.

4. The recombinant protein of any one of embodiments 1 or 2, wherein the two or more amino acid modifications does not include amino acid modifications at amino acid positions corresponding to amino acid positions of 371, 373, and 375 in SEQ ID NO:104.

5. The recombinant protein of any one of embodiments 1 to 4, wherein the two or more amino acid modifications further comprises the following amino acid modification at the amino acid position corresponding to the indicated amino acid positions of SEQ ID NO: 104: G339D or G339H.

6. The recombinant protein of any one of embodiments 1 to 5, wherein the two or more amino acid modifications comprise the following amino acid modifications at amino acid positions corresponding to the indicated amino acid positions of SEQ ID NO: 104: A67V, HV69-70 deletion, T95I, G142D, VYY143-145 deletion, N211 deletion, L212I, ins214EPE, G339D, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F.

7. The recombinant protein of any one of embodiments 1 to 5, wherein the two or more amino acid modifications further comprise one or more of the following amino acid modifications at amino acid positions corresponding to the indicated amino acid positions of SEQ ID NO: 104: T19I, del24-26 (LPP), T376A, D405N, R408S, and/or Q498R.

8. The recombinant protein of any one of embodiment 1 to 5, wherein the two or more amino acid modifications further comprise the following amino acid modifications at amino acid positions corresponding to the indicated amino acid positions of SEQ ID NO: 104: T19I, del24-26 (LPP), T376A, D405N, R408S, and Q498R.

9 The recombinant protein of any one of embodiments 1 to 5, 7, or 8, wherein the two or more amino acid modifications further comprise the following amino acid modification at the amino acid position corresponding to the indicated amino acid position of SEQ ID NO: 104: V213G or V213E.

10. The recombinant protein of any one of embodiments 1 to 5, wherein the two or more amino acid modifications comprise the following amino acid modifications at amino acid positions corresponding to the indicated amino acid positions of SEQ ID NO: 104: T19I, del24-26 (LPP), A27S, G142D, V213G, G339D, T376A, D405N, R408S, K417N, N440K, S477N, T478K, E484A, Q493R, Q498R, N501Y, Y505H, D614G, H655Y, N679K, P681H, N764K, D796Y, Q954H, and N969K.

11. The recombinant protein of any one of embodiments 1 to 5, wherein the two or more amino acid modifications comprise the following amino acid modifications at the amino acid positions corresponding to the indicated amino acid positions of SEQ ID NO: 104: T19I, del24-26 (LPP), A27S, del69-70, G142D, V213G, G339D, R346T, T376A, D405N, R408S, K417N, N440K, K444T, L452R, N460K, S477N, T478K, E484A, F486V, Q498R, N501Y, Y505H, D614G, H655Y, N679K, P681H, N764K, D796Y, Q954H, and N969K.

12. The recombinant protein of any one of embodiments 1 to 5, wherein the two or more amino acid modifications comprise the following amino acid modifications at the amino acid positions corresponding to the indicated amino acid positions of SEQ ID NO:104: T19I, del24-26 (LPP), A27S, V83A, G142D, del144, H146Q, Q183E, V213E, G252V, G339H, R346T, L368I, T376A, D405N, R408S, K417N, N440K, V445P, G446S, N460K, S477N, T478K, E484A, F486P, F490S, Q498R, N501Y, Y505H, D614G, H655Y, N679K, P681H, N764K, D796Y, Q954H, and N969K.

13. The recombinant protein of any one of embodiments 1 to 3, wherein the two or more amino acid modifications does not include amino acid modification at the amino acid position corresponding to amino acid position of 452 in SEQ ID NO: 104.

14. The recombinant protein of any one of embodiments 1 to 5, or 13, wherein the two or more amino acid modifications comprise the following amino acid modifications at the amino acid positions corresponding to the indicated amino acid positions of SEQ ID NO: 104: T19I, del24-26 (LPP), A27S, del69-70 (HV), G142D, V213G, G339D, T376A, D405N, R408S, K417N, N440K, S477N, L452R, T478K, E484A, F486V, Q498R, N501Y, Y505H, D614G, H655Y, N679K, P681H, N764K, D796Y, Q954H, and N969K.

15. A recombinant protein comprising a derivative of a SARS-CoV-2 Omicron spike protein ectodomain, wherein the derivative comprises the ectodomain of the amino acid sequence of SEQ ID NO: 104 without the signal peptide and with amino acid modifications, wherein the amino acid modifications comprise: (1) an amino acid substitution at amino acid residues corresponding to amino acid residues 682 to 685 (RRAR) of SEQ ID NO: 104 to a single alanine; (2) amino acid substitutions at amino acid residues corresponding to the following amino acid residues of SEQ ID NO: 104: F817P, A892P, A899P, A942P, K986P, and V987P; and (3) 18 or more amino acid modifications to the amino acid sequence of the ectodomain of SEQ ID NO: 104 to amino acid residues found at the corresponding amino acid positions in the Omicron spike protein ectodomain.

16. The recombinant protein of embodiment 15, wherein the 18 or more amino acid modifications do not include amino acid modifications at the amino acid positions corresponding to the amino acid positions 371 and 375 of SEQ ID NO:104.

17. The recombinant protein of embodiment 15, wherein the 18 or more amino acid modifications do not include amino acid modifications at the amino acid positions corresponding to the amino acid positions 371, 373, and 375 of SEQ ID NO: 104.

18. The recombinant protein of any one of embodiments 15 to 17, wherein the 18 or more amino acid modifications comprise the following amino acid modifications at amino acid positions corresponding to the indicated amino acid positions of SEQ ID NO:104: A67V, HV69-70 deletion, T95I, G142D, VYY143-145 deletion, N211 deletion, L212I, ins214EPE, G339D, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F.

19. The recombinant protein of any one of embodiments 15 to 17, wherein the 18 or more amino acid modifications comprise the following amino acid modifications at amino acid positions corresponding to the indicated amino acid positions of SEQ ID NO: 104: T19I, del24-26 (LPP), A27S, G142D, V213G, G339D, T376A, D405N, R408S, K417N, N440K, S477N, T478K, E484A, Q493R, Q498R, N501Y, Y505H, D614G, H655Y, N679K, P681H, N764K, D796Y, Q954H, and N969K.

20. The recombinant protein of any one of embodiments 15 to 17, wherein the 18 or more amino acid modifications comprise the following amino acid modifications at amino acid positions corresponding to the indicated amino acid positions of SEQ ID NO:104: T19I, del24-26 (LPP), A27S, del69-70, G142D, V213G, G339D, R346T, T376A, D405N, R408S, K417N, N440K, K444T, L452R, N460K, S477N, T478K, E484A, F486V, Q498R, N501Y, Y505H, D614G, H655Y, N679K, P681H, N764K, D796Y, Q954H, and N969K.

21. The recombinant protein of any one of embodiments 15 to 17, wherein the 18 or more amino acid modifications comprise the following amino acid modifications at amino acid positions corresponding to the indicated amino acid positions of SEQ ID NO: 104: T19I, del24-26 (LPP), A27S, V83A, G142D, del144, H146Q, Q183E, V213E, G252V, G339H, R346T, L368I, T376A, D405N, R408S, K417N, N440K, V445P, G446S, N460K, S477N, T478K, E484A, F486P, F490S, Q498R, N501Y, Y505H, D614G, H655Y, N679K, P681H, N764K, D796Y, Q954H, and N969K.

22. The recombinant protein of embodiment 15, wherein the 18 or more amino acid modifications comprise the following amino acid modifications at amino acid positions corresponding to the indicated amino acid positions of SEQ ID NO: 104: T19I, del24-26 (LPP), A27S, del69-70, G142D, V213G, G339D, R346T, S371F, S373P, S375F, T376A, D405N, R408S, K417N, N440K, K444T, L452R, N460K, S477N, T478K, E484A, F486V, Q498R, N501Y, Y505H, D614G, H655Y, N679K, P681H, N764K, D796Y, Q954H, and N969K.

23. The recombinant protein of embodiment 15, wherein the 18 or more amino acid modifications comprise the following amino acid modifications at the amino acid positions corresponding to the indicated amino acid positions of SEQ ID NO: 104: T19I, del24-26 (LPP), A27S, V83A, G142D, del144, H146Q, Q183E, V213E, G252V, G339H, R346T, L368I, S371F, S373P, S375F, T376A, D405N, R408S, K417N, N440K, V445P, G446S, N460K, S477N, T478K, E484A, F486P, F490S, Q498R, N501Y, Y505H, D614G, H655Y, N679K, P681H, N764K, D796Y, Q954H, and N969K.

24. The recombinant protein of any one of embodiments 15 to 17, wherein the 18 or more amino acid modifications does not include amino acid modification at the amino acid position corresponding to amino acid position of 452 in SEQ ID NO: 104.

25. The recombinant protein of any one of embodiments 15 to 17, or 24, wherein the 18 or more amino acid modifications comprise the following amino acid modifications at amino acid positions corresponding to the indicated amino acid positions of SEQ ID NO:104: T19I, del24-26 (LPP), A27S, del69-70 (HV), G142D, V213G, G339D, T376A, D405N, R408S, K417N, N440K, S477N, L452R, T478K, E484A, F486V, Q498R, N501Y, Y505H, D614G, H655Y, N679K, P681H, N764K, D796Y, Q954H, and N969K.

26. The recombinant protein of embodiment 15, wherein the derivative of the ectodomain comprises the amino acid sequence of SEQ ID NO: 103, 35, 85, 47, 59, 91, 97, 19, 21, 23, 41, 53, 65, 71, or 79.

27. A recombinant protein comprising a derivative of the ectodomain of a SARS-CoV-2 variant, wherein the ectodomain comprises an amino acid sequence that is at least 90% identical to the amino acid sequence of SEQ ID NO: 103, 35, 85, 47, 59, 91, 97, 19, 21, 23, 41, 53, 65, 71, 79, 33, 39, 45, 51, 57, 63, 69, 77, 83, 89, 95 or 101.

28. The recombinant protein of embodiment 27, wherein the derivative of the ectodomain comprises: (1) alanine at amino acid residues corresponding to amino acid residues 682 to 685 (RRAR) of the amino acid sequence of SEQ ID NO: 104; (2) proline at amino acid residues corresponding to the following amino acid residues of the amino acid sequence of SEQ ID NO: 104: F817, A892, A899, A942, K986, and V987; and (3) two or more of the following amino acid residues at amino acid positions corresponding to the indicated amino acid positions of the amino acid sequence of SEQ ID NO:104: 440K, 477N, 505H, 679K, 764K, 796Y, 954H, and/or 969K.

29 The recombinant protein of embodiment 27, wherein the ectodomain comprises the amino acid sequence of SEQ ID NO: 103, 35, 85, 47, 59, 91, 97, 19, 21, 23, 41, 53, 65, 71, 79, 33, 39, 45, 51, 57, 63, 69, 77, 83, 89, 95 or 101.

30. The recombinant protein of any one of embodiments 1 to 26, wherein the protein further comprises a signal peptide.

31. The recombinant protein of embodiment 27, wherein the signal peptide comprises the amino acid sequence of SEQ ID NO:29.

32. The recombinant protein of any one of embodiments 1 to 31, wherein the protein further comprises the transmembrane and cytoplasmic domains of NDV F protein.

33. The recombinant protein of any one of embodiments 1 to 32, wherein the protein further comprises a linker and the transmembrane and cytoplasmic domains of NDV F protein.

34. The recombinant protein of embodiment 32 or 33, wherein the transmembrane and cytoplasmic domains of NDV F protein comprises the amino acid sequence of SEQ ID NO: 5.

35. A polynucleotide comprising a nucleotide sequence encoding the protein of any one of embodiments 1 to 31.

36. The polynucleotide of embodiment 35, which comprises the nucleotide sequence of SEQ ID NO: 34, 84, 102, 90, 96, 46, 58, 18, 20, 22, 40, 52, 64, 70, 78, 32, 82, 100, 88, 94, 44, 56, 38, 50, 62, 68, or 76.

37. A polynucleotide comprising a nucleotide sequence encoding the protein of any one of embodiments 32 to 33.

38. The polynucleotide of embodiment 37, which comprises the nucleotide sequence of SEQ ID NO: 30, 42, 54, 66, 80, 86, 92, 98, 6, 7, 10, 11, 14, 15, 36, 48, 60 or 74.

39. The polynucleotide of embodiment 38, wherein the nucleotide sequence encodes the amino acid sequence of SEQ ID NO: 31, 43, 55, 67, 81, 87, 93, 99, 8, 9, 12, 13, 16, 17, 37, 49, 61, or 75.

40. A vector comprising the polynucleotide of any one of embodiments 35 to 39.

41. The vector of embodiment 40, which is a plasmid or a viral vector.

42. A transgene comprising a nucleotide sequence encoding a chimeric F protein, wherein the chimeric F protein comprises:
(a) the amino acid sequence of SEQ ID NO: 31, 43, 55, 67, 81, 87, 93, 99, 8, 12, 16, 37, 49, 61, or 75 without the signal peptide;
(b) the amino acid sequence of SEQ ID NO: 31, 43, 55, 67, 81, 87, 93, 99, 8, 12, 16, 37, 49, 61, or 75;
(c) an amino acid sequence that is at least 90% identical to SEQ ID NO: 31, 43, 55, 67, 81, 87, 93, 99, 8, 12, 16, 37, 49, 61, or 75 without the signal peptide; or
(d) an amino acid sequence that is at least 90% identical to SEQ ID NO: 31, 43, 55, 67, 81, 87, 93, 99, 8, 12, 16, 37, 49, 61, or 75.

43. The transgene of embodiment 42, wherein the chimeric F protein comprises an amino acid sequence that is at least 90% identical to SEQ ID NO: 31, 43, 55, 67, 81, 87, 93, 99, 8, 12, 16, 37, 49, 61, or 75 without the signal peptide.

44. The transgene of embodiment 42, wherein the chimeric F protein comprises an amino acid sequence that is at least 90% identical to SEQ ID NO: 31, 43, 55, 67, 81, 87, 93, 99, 8, 12, 16, 37, 49, 61, or 75.

45. The transgene of embodiment 42, wherein the chimeric F protein comprises an amino acid sequence that is at least 95%, at least 98%, or at least 99% identical to SEQ ID NO: 31, 43, 55, 67, 81, 87, 93, 99, 8, 12, 16, 37, 49, 61, or 75 without the signal peptide.

46. The transgene of embodiment 42, wherein the chimeric F protein comprises an amino acid sequence that is at least 95%, at least 98%, or at least 99% identical to SEQ ID NO: 31, 43, 55, 67, 81, 87, 93, 99, 8, 12, 16, 37, 49, 61, or 75.

47. The transgene of embodiment 42, wherein the chimeric F protein comprises the amino acid sequence of SEQ ID NO: 31, 43, 55, 67, 81, 87, 93, 99, 8, 12, 16, 37, 49, 61, or 75 without the signal peptide.

48. The transgene of embodiment 42, wherein the chimeric F protein comprises the amino acid sequence of SEQ ID NO: 31, 43, 55, 67, 81, 87, 93, 99, 8, 12, 16, 37, 49, 61, or 75.

49. A transgene comprising a nucleotide sequence encoding a chimeric F protein, wherein the chimeric F protein comprises a derivative of a SARS-CoV-2 Omicron virus spike protein ectodomain and NDV F protein transmembrane and cytoplasmic domains, and wherein the derivative of the SARS-CoV-2 Omicron virus spike protein ectodomain comprises:
(a) the amino acid sequence of SEQ ID NO: 35, 85, 103, 91, 97, 47, 59, 19, 21, 23, 41, 53, 65, 71, or 79;
(b) the amino acid sequence of SEQ ID NO: 33, 83, 101, 89, 95, 45, 57, 39, 51, 63, 69, or 77;
(c) an amino acid sequence at least 90% identical to the amino acid sequence of SEQ ID NO: 35, 85, 103, 91, 97, 47, 59, 19, 21, 23, 41, 53, 65, 71, or 79; or
(d) an amino acid sequence at least 90% identical to the amino acid sequence of SEQ ID NO: 33, 83, 101, 89, 95, 45, 57, 39, 51, 63, 69, or 77.

50. The transgene of embodiment 49, wherein the derivative of the SARS-CoV-2 Omicron virus spike protein ectodomain comprises an amino acid sequence at least 90% identical to the amino acid sequence of SEQ ID NO: 35, 85, 103, 91, 97, 47, 59, 19, 21, 23, 41, 53, 65, 71, or 79.

51. The transgene of embodiment 49, wherein the derivative of the SARS-CoV-2 Omicron virus spike protein ectodomain comprises an amino acid sequence at least 90% identical to the amino acid sequence of SEQ ID NO: 33, 83, 101, 89, 95, 45, 57, 39, 51, 63, 69, or 77.

52. The transgene of embodiment 49, wherein the derivative of the SARS-CoV-2 Omicron virus spike protein ectodomain comprises an amino acid sequence at least 95%, at least 98%, or at least 99% identical to the amino acid sequence of SEQ ID NO: 35, 85, 103, 91, 97, 47, 59, 19, 21, 23, 41, 53, 65, 71, or 79.

53. The transgene of embodiment 49, wherein the derivative of the SARS-CoV-2 Omicron virus spike protein ectodomain comprises an amino acid sequence at least 95%, at least 98%, or at least 99% identical to the amino acid sequence of SEQ ID NO: 33, 83, 101, 89, 95, 45, 57, 39, 51, 63, 69, or 77.

54. The transgene of embodiment 49, wherein the derivative of the SARS-CoV-2 Omicron virus spike protein ectodomain comprises the amino acid sequence of SEQ ID NO: 35, 85, 103, 91, 97, 47, 59, 19, 21, 23, 41, 53, 65, 71, or 79.

55. The transgene of embodiment 49, wherein the derivative of the SARS-CoV-2 Omicron virus spike protein ectodomain comprises the amino acid sequence of SEQ ID NO: 33, 83, 101, 89, 95, 45, 57, 39, 51, 63, 69, or 77.

56. A transgene comprising:
(a) the nucleotide sequence of SEQ ID NO: 30, 42, 54, 66, 80, 86, 92, 98, 6, 10, 14, 36, 48, 60, or 74 without the nucleotide sequence encoding the signal peptide;
(b) the nucleotide sequence of SEQ ID NO: 30, 42, 54, 66, 80, 86, 92, 98, 6, 10, 14, 36, 48, 60, or 74;
(c) a nucleotide sequence that is at least 80% identical to SEQ ID NO: 30, 42, 54, 66, 80, 86, 92, 98, 6, 10, 14, 36, 48, 60, or 74 without the nucleotide sequence encoding the signal peptide; or
(d) a nucleotide sequence that is at least 80% identical to SEQ ID NO: 30, 42, 54, 66, 80, 86, 92, 98, 6, 10, 14, 36, 48, 60, or 74.

57. The transgene of embodiment 56, which comprises a nucleotide sequence that is at least 80% identical to SEQ ID NO: 30, 42, 54, 66, 80, 86, 92, 98, 6, 10, 14, 36, 48, 60, or 74 without the nucleotide sequence encoding the signal peptide.

58. The transgene of embodiment 56, which comprises a nucleotide sequence that is at least 80% identical to SEQ ID NO: 30, 42, 54, 66, 80, 86, 92, 98, 6, 10, 14, 36, 48, 60, or 74.

59. The transgene of embodiment 56, which comprises a nucleotide sequence that is at least 85%, at least 90%, at least 95%, at least 98%, or at least 99% identical to SEQ ID NO: 30, 42, 54, 66, 80, 86, 92, 98, 6, 10, 14, 36, 44, 48, 60, or 74 without the nucleotide sequence encoding the signal peptide.

60. The transgene of embodiment 56, which comprises a nucleotide sequence that is at least 85%, at least 90%, at least 95%, at least 98%, or at least 99% identical to SEQ ID NO: 30, 42, 54, 66, 80, 86, 92, 98, 6, 10, 14, 36, 48, 60, or 74.

61. The transgene of embodiment 56, which comprises the nucleotide sequence of SEQ ID NO: 30, 42, 54, 66, 80, 86, 92, 98, 6, 10, 14, 36, 48, 60, or 74 without the nucleotide sequence encoding the signal peptide.

62. The transgene of embodiment 56, which comprises the nucleotide sequence of SEQ ID NO: 30, 42, 54, 66, 80, 86, 92, 98, 6, 10, 14, 36, 48, 60, or 74.

63. A transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises:
(a) the nucleotide sequence of SEQ ID NO: 34, 84, 102, 90, 96, 46, 58, 18, 20, 22, 40, 52, 64, 70, or 78;
(b) the nucleotide sequence of SEQ ID NO: 32, 82, 100, 88, 94, 44, 56, 38, 50, 62, 68, or 76;
(c) a nucleotide sequence that is at least 80% identical to the nucleotide sequence of SEQ ID NO: 34, 84, 102, 90, 96, 46, 58, 18, 20, 22, 40, 52, 64, 70, or 78; or
(d) a nucleotide sequence that is at least 80% identical to the nucleotide sequence of SEQ ID NO: 32, 82, 100, 88, 94, 44, 56, 38, 50, 62, 68, or 76.

64. The transgene of embodiment 63, wherein the nucleotide sequence encoding the derivative of the SARS-CoV-2 Omicron virus spike protein ectodomain comprises a nucleotide sequence that is at least 80% identical to the nucleotide sequence of SEQ ID NO: 34, 84, 102, 90, 96, 46, 58, 18, 20, 22, 40, 52, 64, 70, or 78.

65. The transgene of embodiment 63, wherein the nucleotide sequence encoding the derivative of the SARS-CoV-2 Omicron virus spike protein ectodomain comprises a nucleotide sequence that is at least 80% identical to the nucleotide sequence of SEQ ID NO: 32, 82, 100, 88, 94, 44, 56, 38, 50, 62, 68, or 76.

66. The transgene of embodiment 63, wherein the nucleotide sequence encoding the derivative of the SARS-CoV-2 Omicron virus spike protein ectodomain comprises a nucleotide sequence that is at least 85%, at least 90%, at least 95%, at least 98%, or at least 99% identical to the nucleotide sequence of SEQ ID NO: 34, 84, 102, 90, 96, 46, 58, 18, 20, 22, 40, 52, 64, 70, or 78.

67. The transgene of embodiment 63, wherein the nucleotide sequence encoding the derivative of the SARS-CoV-2 Omicron virus spike protein ectodomain comprises a nucleotide sequence that is at least 85%, at least 90%, at least 95%, at least 98%, or at least 99% identical to the nucleotide sequence of SEQ ID NO: 32, 82, 100, 88, 94, 44, 56, 38, 50, 62, 68, or 76.

68. The transgene of embodiment 63, wherein the nucleotide sequence encoding the derivative of the SARS-CoV-2 Omicron virus spike protein ectodomain comprises the nucleotide sequence of SEQ ID NO: 34, 84, 102, 90, 96, 46, 58, 18, 20, 22, 40, 52, 64, 70, or 78.

69. The transgene of embodiment 63, wherein the nucleotide sequence encoding the derivative of the SARS-CoV-2 Omicron virus spike protein ectodomain comprises the nucleotide sequence of SEQ ID NO: 32, 82, 100, 88, 94, 44, 56, 38, 50, 62, 68, or 76.

70. A transgene comprising:
(a) an RNA sequence corresponding to the negative sense of the cDNA sequence of SEQ ID NO: 30, 42, 54, 66, 80, 86, 92, 98, 6, 10, 14, 36, 48, 60, or 74 without the nucleotide sequence encoding the signal peptide;
(b) an RNA sequence corresponding to the negative sense of the cDNA sequence of SEQ ID NO: 30, 42, 54, 66, 80, 86, 92, 98, 6, 10, 14, 36, 48, 60, or 74;
(c) an RNA sequence corresponding to the negative sense of the cDNA sequence of a nucleotide sequence that is at least 80% identical to the cDNA sequence of SEQ ID NO: 30, 42, 54, 66, 80, 86, 92, 98, 6, 10, 14, 36, 48, 60, or 74 without the nucleotide sequence encoding the signal peptide; or
(d) an RNA sequence corresponding to the negative sense of the cDNA sequence of a nucleotide sequence that is at least 80% identical to the cDNA sequence of SEQ ID NO: 30, 42, 54, 66, 80, 86, 92, 98, 6, 10, 14, 36, 48, 60, or 74.

71. The transgene of embodiment 70, which comprises an RNA sequence corresponding to the negative sense of the cDNA sequence of a nucleotide sequence that is at least 80% identical to the cDNA sequence of SEQ ID NO: 30, 42, 54, 66, 80, 86, 92, 98, 6, 10, 14, 36, 48, 60, or 74 without the nucleotide sequence encoding the signal peptide.

72. The transgene of embodiment 70, which comprises an RNA sequence corresponding to the negative sense of the cDNA sequence of a nucleotide sequence that is at least 80% identical to the cDNA sequence of SEQ ID NO: 30, 42, 54, 66, 80, 86, 92, 98, 6, 10, 14, 36, 48, 60, or 74.

73. The transgene of embodiment 70, which comprises an RNA sequence corresponding to the negative sense of a nucleotide sequence that is at least 85%, at least 90%, at least 95%, at least 98%, or at least 99% identical to the cDNA sequence of SEQ ID NO: 30, 42, 54, 66, 80, 86, 92, 98, 6, 10, 14, 36, 48, 60, or 74 without the nucleotide sequence encoding the signal peptide.

74. The transgene of embodiment 70, which comprises an RNA sequence corresponding to the negative sense of a nucleotide sequence that is at least 85%, at least 90%, at least 95%, at least 98%, or at least 99% identical to the cDNA sequence of SEQ ID NO: 30, 42, 54, 66, 80, 86, 92, 98, 6, 10, 14, 36, 48, 60, or 74.

75. The transgene of embodiment 70, which comprises an RNA sequence corresponding to the negative sense of the cDNA sequence of SEQ ID NO: 30, 42, 54, 66, 80, 86, 92, 98, 6, 10, 14, 36, 48, 60, or 74 without the nucleotide sequence encoding the signal peptide.

76. The transgene of embodiment 70, which comprises an RNA sequence corresponding to the negative sense of the cDNA sequence of SEQ ID NO: 30, 42, 54, 66, 80, 86, 92, 98, 6, 10, 14, 36, 48, 60, or 74.

77. A transgene comprising a polynucleotide encoding a chimeric F protein, wherein the chimeric F protein comprises a derivative of a SARS-CoV-2 Omicron virus spike protein ectodomain and NDV F protein transmembrane and cytoplasmic domains, and wherein the nucleotide sequence encoding the derivative of the SARS-CoV-2 Omicron virus spike protein ectodomain comprises:
(a) an RNA sequence corresponding to the negative sense of the cDNA sequence of SEQ ID NO: 34, 84, 102, 90, 96, 46, 58, 18, 20, 22, 40, 52, 64, 70, or 78;
(b) an RNA sequence corresponding to the negative sense of the cDNA sequence of SEQ ID NO: 32, 82, 100, 88, 94, 44, 56, 38, 50, 62, 68, or 76;
(c) an RNA sequence corresponding to the negative sense of the cDNA sequence of a nucleotide sequence that is at least 80% identical to the cDNA sequence of SEQ ID NO: 34, 84, 102, 90, 96, 46, 58, 18, 20, 22, 40, 52, 64, 70, or 78; or
(d) an RNA sequence corresponding to the negative sense of the cDNA sequence of a nucleotide sequence that is at least 80% identical to the cDNA sequence of SEQ ID NO: 32, 82, 100, 88, 94, 44, 56, 38, 50, 62, 68, or 76.

78. The transgene of embodiment 77, wherein the nucleotide sequence encoding the derivative of the SARS-CoV-2 Omicron virus spike protein ectodomain comprises an RNA sequence corresponding to the negative sense of a nucleotide sequence that is at least 80% identical to the cDNA sequence of SEQ ID NO: 34, 84, 102, 90, 96, 46, 58, 18, 20, 22, 40, 52, 64, 70, or 78.

79. The transgene of embodiment 77, wherein the nucleotide sequence encoding the derivative of the SARS-CoV-2 Omicron virus spike protein ectodomain comprises an RNA sequence corresponding to the negative sense of a nucleotide sequence that is at least 80% identical to the cDNA sequence of SEQ ID NO: 32, 82, 100, 88, 94, 44, 56, 38, 50, 62, 68, or 76.

80. The transgene of embodiment 77, wherein the nucleotide sequence encoding the derivative of the SARS-CoV-2 Omicron virus spike protein ectodomain comprises an RNA sequence corresponding to the negative sense of a nucleotide sequence that is at least 85%, at least 90%, at least 95%, at least 98%, or at least 99% identical to the cDNA sequence of SEQ ID NO: 34, 84, 102, 90, 96, 46, 58, 18, 20, 22, 40, 52, 64, 70, or 78.

81. The transgene of embodiment 77, wherein the nucleotide sequence encoding the derivative of the SARS-CoV-2 Omicron virus spike protein ectodomain comprises an RNA sequence corresponding to the negative sense of a nucleotide sequence that is at least 85%, at least 90%, at least 95%, at least 98%, or at least 99% identical to the cDNA sequence of SEQ ID NO: 32, 82, 100, 88, 94, 44, 56, 38, 50, 62, 68, or 76.

82. The transgene of embodiment 77, wherein the nucleotide sequence encoding the derivative of the SARS-CoV-2 Omicron virus spike protein ectodomain comprises an RNA sequence corresponding to the negative sense of the cDNA sequence of SEQ ID NO: 34, 84, 102, 90, 96, 46, 58, 18, 20, 22, 40, 52, 64, 70, or 78.

83. The transgene of embodiment 77, wherein the nucleotide sequence encoding the derivative of the SARS-CoV-2 Omicron virus spike protein ectodomain comprises an RNA sequence corresponding to the negative sense of the cDNA sequence of SEQ ID NO: 32, 82, 100, 88, 94, 44, 56, 38, 50, 62, 68, or 76.

84. The transgene of any one of embodiments 49 to 55, wherein the SARS-CoV-2 Omicron virus spike protein ectodomain is linked via a linker to the NDV F protein transmembrane and cytoplasmic domains.

85. The transgene of embodiment 84, wherein the linker comprises the amino acid sequence of SEQ ID NO:24.

86. The transgene of any one of embodiments 77 to 82, wherein the SARS-CoV-2 Omicron virus spike protein ectodomain is linked via a linker to the NDV F protein transmembrane and cytoplasmic domains.

87. The transgene of embodiment 82, wherein the linker comprises the amino acid sequence of SEQ ID NO:24.

88. The transgene of any one of embodiments 63 to 69, wherein the SARS-CoV-2 Omicron virus spike protein ectodomain is linked via a linker to the NDV F protein transmembrane and cytoplasmic domains.

89. The transgene of embodiment 88, wherein the linker comprises the amino acid sequence of SEQ ID NO:24.

90. The transgene of any one of embodiments 56 to 69, 88 or 89, wherein the transgene further comprises a Newcastle Disease Virus (NDV) gene start sequence.

91. The transgene of any one of embodiments 56 to 69, 88, or 89, wherein the transgene further comprises a Newcastle Disease Virus (NDV) gene end sequence.

92. The transgene of any one of embodiments 56 to 69, 88, 89, or 91, wherein the transgene further comprises the nucleotide sequence of SEQ ID NO:26 and 27.

93. The transgene of any one of embodiments 56 to 69, 88, 89, 91, or 92, wherein the transgene further comprises the nucleotide sequence of SEQ ID NO: 25, SEQ ID NO:28, or SEQ ID NOS: 25 and 28.

94. The transgene of any one of embodiments 42 to 55, or 77 to 87, wherein the transgene further comprises a Newcastle Disease Virus (NDV) gene start sequence.

95. The transgene of any one of embodiments 42 to 55, 77 to 87, or 94, wherein the transgene further comprises a Newcastle Disease Virus (NDV) gene end sequence.

96. The transgene of any one of embodiments 42 to 55, 77 to 87, 94, or 95, wherein the transgene further comprises an RNA sequence corresponding to the negative sense of the cDNA sequence of SEQ ID NO: 25, SEQ ID NO:28, or an RNA sequence corresponding to the negative sense of the cDNA sequence of SEQ ID NOS: 25 and 28.

97. A vector comprising the transgene of any one of embodiments 42 to 96.

98. A nucleotide sequence comprising the transgene of any one of embodiments 42 to 55, 70 to 87, or 94 to 96, and (1) a NDV F transcription unit, (2) a NDV NP transcription unit, (3) a NDV M transcription unit, (4) a NDV L transcription unit, (5) a NDV P transcription unit, and (6) a NDV HN transcription unit.

99. A nucleotide sequence comprising the transgene of any one of embodiments 42 to 55, 70 to 87, or 94 to 96, and (1) a NDV F transcription unit, (2) a NDV NP transcription unit, (3) a NDV M transcription unit, (4) a NDV L transcription unit, (5) a NDV P transcription unit, and (6) a NDV HN transcription unit, wherein the NDV F transcription unit encodes a NDV F protein comprising a leucine to alanine amino acid substitution at the amino residue corresponding to amino acid residue 289 of the LaSota NDV strain.

100. A nucleotide sequence comprising the transgene of any one of embodiments 56 to 69, or 88 to 93, and (1) a NDV F transcription unit, (2) a NDV NP transcription unit, (3) a NDV M transcription unit, (4) a NDV L transcription unit, (5) a NDV P transcription unit, and (6) a NDV HN transcription unit.

101. A nucleotide sequence comprising the transgene of any one of embodiments 56 to 69, or 88 to 93, and (1) a NDV F transcription unit, (2) a NDV NP transcription unit, (3) a NDV M transcription unit, (4) a NDV L transcription unit, (5) a NDV P transcription unit, and (6) a NDV HN transcription unit, wherein the NDV F transcription unit encodes a NDV F protein comprising a leucine to alanine amino acid substitution at the amino residue corresponding to amino acid residue 289 of the LaSota NDV strain.

102. A vector comprising the nucleotide sequence of any one of embodiments 98 to 101.

103. A recombinant Newcastle disease virus (NDV) comprising a packaged genome, wherein the packaged genome comprises the transgene of any one of embodiments 42 to 55, 70 to 87, or 94 to 96.

104. The recombinant NDV of embodiment 103, wherein the NDV virion comprises the chimeric F protein.

105. A recombinant Newcastle disease virus (NDV) comprising a packaged genome, wherein the packaged genome comprises a transgene, wherein the transgene encodes a protein, wherein the protein comprises a derivative of a SARS-CoV-2 Omicron virus spike protein ectodomain, and wherein the derivative of the SARS-CoV-2 Omicron virus spike protein ectodomain comprises an amino acid sequence SEQ ID NO: 35, 85, 103, 91, 97, 47, 59, 19, 21, 23, 41, 53, 65, 71, or 79, or an amino acid sequence at least 90% identical to the amino acid sequence of SEQ ID NO: 35, 85, 103, 91, 97, 47, 59, 19, 21, 23, 41, 53, 65, 71, or 79.

106. The recombinant NDV of embodiment 105, wherein the derivative of the ectodomain comprises an amino acid sequence at least 90% identical to the amino acid sequence of SEQ ID NO: 35, 85, 103, 91, 97, 47, 59, 19, 21, 23, 41, 53, 65, 71, or 79, and wherein the derivative of the ectodomain comprises: (1) alanine at amino acid residues corresponding to amino acid residues 682 to 685 (RRAR) of the amino acid sequence of SEQ ID NO: 104; (2) proline at amino acid residues corresponding to the following amino acid residues of the amino acid sequence of SEQ ID NO: 104: F817, A892, A899, A942, K986, and V987; and (3) two or more of the following amino acid residues at amino acid positions corresponding to the indicated amino acid positions of the amino acid sequence of SEQ ID NO: 104: 440K, 477N, 505H, 679K, 764K, 796Y, 954H, and/or 969K.

107. The recombinant NDV of embodiment 103 to 106, wherein the genome comprises a NDV F transcription unit, a NDV NP transcription unit, a NDV M transcription unit, a NDV L transcription unit, a NDV P transcription unit, and a NDV HN transcription unit.

108. The recombinant NDV of embodiment 103 to 106, wherein the genome comprises a NDV F transcription unit, a NDV NP transcription unit, a NDV M transcription unit, a NDV L transcription unit, a NDV P transcription unit, and a NDV HN transcription unit, and wherein the NDV F transcription unit encodes a NDV F protein comprising a leucine to alanine amino acid substitution at the amino residue corresponding to amino acid residue 289 of the LaSota NDV strain.

109. The recombinant NDV of any one of embodiments 103 to 108, wherein the transgene is between two NDV transcription units of the packaged genome.

110. The recombinant NDV of embodiment 109, wherein the two transcription units of the packaged genome are the transcription units for the NDV P gene and the NDV M gene.

111. The recombinant NDV of embodiment 109, wherein the two transcription units of the packaged genome are the transcription units for the NDV NP gene and the NDV P gene.

112. A recombinant NDV comprising a chimeric F protein, wherein the chimeric F protein comprises the amino acid sequence of SEQ ID NO: 31, 43, 55, 67, 81, 87, 93, 99, 8, 12, 16, 37, 49, 61, or 75, or an amino acid sequence that is at least 90% identical to SEQ ID NO: 31, 43, 55, 67, 81, 87, 93, 99, 8, 12, 16, 37, 49, 61, or 75.

113. The recombinant NDV of embodiment 112, wherein the chimeric F protein comprises an amino acid sequence that is at least 90%, identical to SEQ ID NO: 31, 43, 55, 67, 81, 87, 93, 99, 8, 12, 16, 37, 49, 61, or 75.

114. The recombinant NDV of embodiment 112, wherein the chimeric F protein comprises an amino acid sequence that is at least 95%, at least 98%, or at least 99% identical to SEQ ID NO: 31, 43, 55, 67, 81, 87, 93, 99, 8, 12, 16, 37, 49, 61, or 75.

115. The recombinant NDV of embodiment 112, wherein the chimeric F protein comprises the amino acid sequence of SEQ ID NO: 31, 43, 55, 67, 81, 87, 93, 99, 8, 12, 16, 37, 49, 61, or 75.

116. A recombinant NDV comprising a protein, wherein the protein comprises a derivative of a SARS-CoV-2 Omicron virus spike protein ectodomain, and wherein the derivative of the SARS-CoV-2 Omicron virus spike protein ectodomain comprises an amino acid sequence SEQ ID NO: 35, 85, 103, 91, 97, 47, 59, 19, 21, 23, 41, 53, 65, 71, or 79, or an amino acid sequence at least 90% identical to the amino acid sequence of SEQ ID NO: 35, 85, 103, 91, 97, 47, 59, 19, 21, 23, 41, 53, 65, 71, or 79.

117. The recombinant NDV of embodiment 116, wherein the derivative of the ectodomain comprises an amino acid sequence at least 90% identical to the amino acid sequence of SEQ ID NO: 35, 85, 103, 91, 97, 47, 59, 19, 21, 23, 41, 53, 65, 71, or 79, and wherein the derivative of the ectodomain comprises: (1) alanine at amino acid residues corresponding to amino acid residues 682 to 685 (RRAR) of the amino acid sequence of SEQ ID NO: 104; (2) proline at amino acid residues corresponding to the following amino acid residues of the amino acid sequence of SEQ ID NO: 104: F817, A892, A899, A942, K986, and V987; and (3) two or more of the following amino acid residues at amino acid positions corresponding to the indicated amino acid positions of the amino acid sequence of SEQ ID NO: 104: 440K, 477N, 505H, 679K, 764K, 796Y, 954H, and/or 969K.

118. A recombinant NDV comprising a chimeric F protein, wherein the chimeric F protein comprises a derivative of a SARS-CoV-2 Omicron virus spike protein ectodomain and NDV F protein transmembrane and cytoplasmic domains, and wherein the derivative of the SARS-CoV-2 Omicron virus spike protein ectodomain comprises an amino acid sequence SEQ ID NO: 35, 85, 103, 91, 97, 47, 59, 19, 21, 23, 41, 53, 65, 71, or 79, or an amino acid sequence at least 90% identical to the amino acid sequence of SEQ ID NO: 35, 85, 103, 91, 97, 47, 59, 19, 21, 23, 41, 53, 65, 71, or 79.

119. The recombinant NDV of embodiment 118, wherein the derivative of the SARS-CoV-2 Omicron virus spike protein ectodomain comprises an amino acid sequence at least 90% identical to the amino acid sequence of SEQ ID NO: 35, 85, 103, 91, 97, 47, 59, 19, 21, 23, 41, 53, 65, 71, or 79.

120. The recombinant NDV of embodiment 118, wherein the derivative of the SARS-CoV-2 Omicron virus spike protein ectodomain comprises an amino acid sequence at least 95%, at least 98%, or at least 99% identical to the amino acid sequence of SEQ ID NO: 35, 85, 103, 91, 97, 47, 59, 19, 21, 23, 41, 53, 65, 71, or 79.

121. The recombinant NDV of embodiment 118, wherein the derivative of the SARS-CoV-2 Omicron virus spike protein ectodomain comprises the amino acid sequence of SEQ ID NO: 35, 85, 103, 91, 97, 47, 59, 19, 21, 23, 41, 53, 65, 71, or 79.

122. The recombinant NDV of any one of embodiments 118 to 121, wherein the SARS-CoV-2 Omicron virus spike protein ectodomain is linked via a linker to the NDV F protein transmembrane and cytoplasmic domains.

123. The recombinant NDV of any one of embodiments 103 to 122, which comprises an NDV backbone which is lentogenic.

124. The recombinant NDV of any one of embodiments 103 to 122, which comprises an NDV backbone of LaSota strain.

125. The recombinant NDV of any one of embodiments 103 to 122, which comprises an NDV backbone of Hitchner B1 strain.

126. A composition comprising the recombinant NDV of any one of embodiments 103 to 125, or vector of any one of embodiments 40, 41, or 102.

127. An immunogenic composition comprising the recombinant NDV of any one of embodiments 103 to 125.

128. The immunogenic composition of embodiment 127, wherein the recombinant NDV is inactivated.

129. An immunogenic composition comprising the polynucleotide of any one of embodiments 35 to 39, or vector of any one of embodiments 40, 41, or 102.

130. The immunogenic composition of embodiment 127 or 129, further comprising an adjuvant.

131. A method for inducing an immune response to SARS-CoV-2 Omicron spike protein, comprising administering the immunogenic composition of any one of embodiments 127 to 130 to a subject.

132. A method for preventing COVID-19, or severe COVID-19, comprising administering the immunogenic composition of any one of embodiments 127 to 130 to a subject.

133. A method for immunizing a subject against SARS-CoV-2, comprising administering the immunogenic composition of any one of embodiments 127 to 130 to a subject.

134. The method of any one of embodiments 131 to 133, wherein the composition is administered to the subject intranasally or intramuscularly.

135. The method of any one of embodiments 127 to 134, wherein the subject is a human.

136. The method of any one of embodiments 127 to 135, wherein the subject has been previously vaccinated with a COVID-19 vaccine.

137. The method of any one of embodiments 127 to 136, wherein the subject is administered at least one booster of the immunogenic composition.

138. A kit comprising the recombinant NDV of any one of embodiments 103 to 125.

139. A kit comprising the transgene of any one of embodiments 42 to 96, the polynucleotide of any one of embodiments 35 to 39, the nucleotide sequence of any one of embodiments 98 to 101, the recombinant protein of any one of embodiments 1 to 34, or the vector of embodiment 40, 41, or 102.

140. A cell line, in vitro cell, or chicken embryonated egg comprising the recombinant NDV of any one of embodiments 103 to 125.

141. A cell line, in vitro cell, or chicken embryonated egg comprising the polynucleotide any one of embodiments 35 to 39, or vector of embodiment 40, 41, or 102, the transgene of any one of embodiments 42 to 96, or the nucleotide sequence of any one of embodiments 98 to 101.

142. A cell line, an in vitro cell, or chicken embryonated egg expressing the recombinant protein any one of embodiments 1 to 34.

143. A method for propagating the recombinant NDV of any one of embodiments 103 to 125, the method comprising culturing the cell line, in vitro cell, or embryonated egg of embodiment 140.

144. The method of embodiment 143, wherein the method further comprises isolating the recombinant NDV from the cell line or embryonated egg.

145. A method for detecting the presence of antibody specific to SARS-CoV-2 Omicron spike protein, comprising contacting a specimen with the recombinant NDV of any one of embodiments 103 to 125, or a recombinant protein of any one of embodiments 1 to 34, in an immunoassay.

146. The method of embodiment 145, wherein the specimen is a biological specimen.

147. The method of embodiment 146, wherein the biological specimen is blood, plasma or sera from a subject.

148. The method of embodiment 147, wherein the subject is human.

149. The method of embodiment 145, wherein the specimen is an antibody or antisera.

The invention is not to be limited in scope by the specific embodiments described herein. Indeed, various modifications of the invention in addition to those described will become apparent to those skilled in the art from the foregoing description and accompanying Figures. Such modifications are intended to fall within the scope of the appended claims.

All references cited herein are incorporated herein by reference in their entirety and for all purposes to the same extent as if each individual publication or patent or patent application was specifically and individually indicated to be incorporated by reference in its entirety for all purposes.

1. A recombinant protein comprising a derivative of a severe-acute-respiratory-syndrome-related coronavirus 2 (SARS-CoV-2) Omicron spike protein ectodomain, wherein
(a) the derivative comprises the ectodomain of the amino acid sequence of SEQ ID NO:104 without the signal peptide and with amino acid modifications, wherein the amino acid modifications comprise: (1) an amino acid substitution at amino acid residues corresponding to amino acid residues 682 to 685 (RRAR) of SEQ ID NO:104 to a single alanine; (2) amino acid substitutions at amino acid residues corresponding to the following amino acid residues of SEQ ID NO: 104: F817P, A892P, A899P, A942P, K986P, and V987P; and (3) two or more amino acid modifications to the amino acid sequence of the ectodomain of SEQ ID NO:104 to amino acid residues found at the corresponding amino acid positions in the Omicron spike protein ectodomain, wherein the two or more amino acid modifications comprise two or more amino acid modifications at amino acid positions corresponding to the indicated amino acid positions of SEQ ID NO: 104: N440K, S477N, Y505H, N679K, N764K, D796Y, Q954H, and/or N969K;
(b) the derivative comprises the ectodomain of the amino acid sequence of SEQ ID NO:104 without the signal peptide and with amino acid modifications, wherein the amino acid modifications comprise: (1) an amino acid substitution at amino acid residues corresponding to amino acid residues 682 to 685 (RRAR) of SEQ ID NO: 104 to a single alanine; (2) amino acid substitutions at amino acid residues corresponding to the following amino acid residues of SEQ ID NO:104: F817P, A892P, A899P, A942P, K986P, and V987P; and (3) 18 or more amino acid modifications to the amino acid sequence of the ectodomain of SEQ ID NO: 104 to amino acid residues found at the corresponding amino acid positions in the Omicron spike protein ectodomain; or
(c) the ectodomain comprises an amino acid sequence that is at least 90% identical to the amino acid sequence of SEQ ID NO: 103, 35, 85, 47, 59, 91, 97, 19, 21, 23, 41, 53, 65, 71, 79, 33, 39, 45, 51, 57, 63, 69, 77, 83, 89, 95 or 101.

2. The recombinant protein of claim 1, wherein
(i) the two or more amino acid modifications comprise the following amino acid modifications at amino acid positions corresponding to the indicated amino acid positions of SEQ ID NO: 104: N440K, S477N, Y505H, N679K, N764K, D796Y, Q954H, and N969K;
(ii1) the two or more amino acid modifications does not include amino acid modifications at amino acid positions corresponding to amino acid positions of 371 and 375 in SEQ ID NO: 104, or (ii2) the two or more amino acid modifications does not include amino acid modifications at amino acid positions corresponding to amino acid positions of 371, 373, and 375 in SEQ ID NO: 104; or
(iii) a combination thereof.

3. (canceled)

4. (canceled)

5. (canceled)

6. The recombinant protein of claim 1, wherein
(i) the two or more amino acid modifications further comprises the following amino acid modification at the amino acid position corresponding to the indicated amino acid positions of SEQ ID NO: 104: G339D or G339H;
(ii) one of:
(1) the two or more amino acid modifications comprise the following amino acid modifications at amino acid positions corresponding to the indicated amino acid positions of SEQ ID NO: 104: A67V, HV69-70 deletion, T95I, G142D, VYY143-145 deletion, N211 deletion, L212I, ins214EPE, G339D, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F;
(2) the two or more amino acid modifications comprise the following amino acid modifications at amino acid positions corresponding to the indicated amino acid positions of SEQ ID NO: 104: T19I, del24-26 (LPP), A27S, G142D, V213G, G339D, T376A, D405N, R408S, K417N, N440K, S477N, T478K, E484A, Q493R, Q498R, N501Y, Y505H, D614G, H655Y, N679K, P681H, N764K, D796Y, Q954H, and N969K;
(3) the two or more amino acid modifications comprise the following amino acid modifications at the amino acid positions corresponding to the indicated amino acid positions of SEQ ID NO: 104: T19I, del24-26 (LPP), A27S, del69-70, G142D, V213G, G339D, R346T, T376A, D405N, R408S, K417N, N440K, K444T, L452R, N460K, S477N, T478K, E484A, F486V, Q498R, N501Y, Y505H, D614G, H655Y, N679K, P681H, N764K, D796Y, Q954H, and N969K; or
(4) the two or more amino acid modifications comprise the following amino acid modifications at the amino acid positions corresponding to the indicated amino acid positions of SEQ ID NO: 104: T19I, del24-26 (LPP), A27S, V83A, G142D, del144, H146Q, Q183E, V213E, G252V, G339H, R346T, L368I, T376A, D405N, R408S, K417N, N440K, V445P, G446S, N460K, S477N, T478K, E484A, F486P, F490S, Q498R, N501Y, Y505H, D614G, H655Y, N679K, P681H, N764K, D796Y, Q954H, and N969K; or
(iii) a combination thereof.

7. The recombinant protein of claim 1, wherein
(i) the two or more amino acid modifications further comprises the following amino acid modification at the amino acid position corresponding to the indicated amino acid positions of SEQ ID NO: 104: G339D or G339H;
(ii1) the two or more amino acid modifications further comprise one or more of the following amino acid modifications at amino acid positions corresponding to the indicated amino acid positions of SEQ ID NO:104: T19I, del24-26 (LPP), T376A, D405N, R408S, and/or Q498R, or (ii2) the two or more amino acid modifications further comprise the following amino acid modifications at amino acid positions corresponding to the indicated amino acid positions of SEQ ID NO: 104: T19I, del24-26 (LPP), T376A, D405N, R408S, and Q498R;
(iii) the two or more amino acid modifications further comprise the following amino acid modification at the amino acid position corresponding to the indicated amino acid position of SEQ ID NO: 104: V213G or V213E; or
(iv) a combination thereof.

8. (canceled)

9. (canceled)

10. (canceled)

11. (canceled)

12. (canceled)

13. The recombinant protein of claim 1, wherein
(i) the two or more amino acid modifications does not include amino acid modification at the amino acid position corresponding to amino acid position of 452 in SEQ ID NO:104;
(ii) the two or more amino acid modifications comprise the following amino acid modifications at the amino acid positions corresponding to the indicated amino acid positions of SEQ ID NO: 104: T19I, del24-26 (LPP), A27S, del69-70 (HV), G142D, V213G, G339D, T376A, D405N, R408S, K417N, N440K, S477N, L452R, T478K, E484A, F486V, Q498R, N501Y, Y505H, D614G, H655Y, N679K, P681H, N764K, D796Y, Q954H, and N969K; or
(iii) a combination thereof.

14. (canceled)

15. (canceled)

16. The recombinant protein of claim 1, wherein
(i1) the 18 or more amino acid modifications do not include amino acid modifications at the amino acid positions corresponding to the amino acid positions 371 and 375 of SEQ ID NO: 104, or (i2) the 18 or more amino acid modifications do not include amino acid modifications at the amino acid positions corresponding to the amino acid positions 371, 373, and 375 of SEQ ID NO:104;
(ii) the 18 or more amino acid modifications comprise the following amino acid modifications at amino acid positions corresponding to the indicated amino acid positions of SEQ ID NO: 104: A67V, HV69-70 deletion, T95I, G142D, VYY143-145 deletion, N211 deletion, L212I, ins214EPE, G339D, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F;
(iii) the 18 or more amino acid modifications comprise the following amino acid modifications at amino acid positions corresponding to the indicated amino acid positions of SEQ ID NO: 104: T19I, del24-26 (LPP), A27S, G142D, V213G, G339D, T376A, D405N, R408S, K417N, N440K, S477N, T478K, E484A, Q493R, Q498R, N501Y, Y505H, D614G, H655Y, N679K, P681H, N764K, D796Y, Q954H, and N969K;
(iv) the 18 or more amino acid modifications comprise the following amino acid modifications at amino acid positions corresponding to the indicated amino acid positions of SEQ ID NO: 104: T19I, del24-26 (LPP), A27S, del69-70, G142D, V213G, G339D, R346T, T376A, D405N, R408S, K417N, N440K, K444T, L452R, N460K, S477N, T478K, E484A, F486V, Q498R, N501Y, Y505H, D614G, H655Y, N679K, P681H, N764K, D796Y, Q954H, and N969K;
(v) the 18 or more amino acid modifications comprise the following amino acid modifications at amino acid positions corresponding to the indicated amino acid positions of SEQ ID NO: 104: T19I, del24-26 (LPP), A27S, V83A, G142D, del144, H146Q, Q183E, V213E, G252V, G339H, R346T, L368I, T376A, D405N, R408S, K417N, N440K, V445P, G446S, N460K, S477N, T478K, E484A, F486P, F490S, Q498R, N501Y, Y505H, D614G, H655Y, N679K, P681H, N764K, D796Y, Q954H, and N969K; or
(vi) a combination thereof.

17. (canceled)

18. (canceled)

19. (canceled)

20. (canceled)

21. (canceled)

22. The recombinant protein of claim 1, wherein
(i) the 18 or more amino acid modifications comprise the following amino acid modifications at amino acid positions corresponding to the indicated amino acid positions of SEQ ID NO: 104: T19I, del24-26 (LPP), A27S, del69-70, G142D, V213G, G339D, R346T, S371F, S373P, S375F, T376A, D405N, R408S, K417N, N440K, K444T, L452R, N460K, S477N, T478K, E484A, F486V, Q498R, N501Y, Y505H, D614G, H655Y, N679K, P681H, N764K, D796Y, Q954H, and N969K;
(ii) the 18 or more amino acid modifications comprise the following amino acid modifications at the amino acid positions corresponding to the indicated amino acid positions of SEQ ID NO: 104: T19I, del24-26 (LPP), A27S, V83A, G142D, del144, H146Q, Q183E, V213E, G252V, G339H, R346T, L368I, S371F, S373P, S375F, T376A, D405N, R408S, K417N, N440K, V445P, G446S, N460K, S477N, T478K, E484A, F486P, F490S, Q498R, N501Y, Y505H, D614G, H655Y, N679K, P681H, N764K, D796Y, Q954H, and N969K; or
(iii) the derivative of the ectodomain comprises the amino acid sequence of SEQ ID NO: 103, 35, 85, 47, 59, 91, 97, 19, 21, 23, 41, 53, 65, 71, or 79.

23. (canceled)

24. The recombinant protein of claim 1, wherein
(i1) the 18 or more amino acid modifications do not include amino acid modifications at the amino acid positions corresponding to the amino acid positions 371 and 375 of SEQ ID NO: 104, or (i2) the 18 or more amino acid modifications do not include amino acid modifications at the amino acid positions corresponding to the amino acid positions 371, 373, and 375 of SEQ ID NO: 104;
(ii) the 18 or more amino acid modifications does not include amino acid modification at the amino acid position corresponding to amino acid position of 452 in SEQ ID NO: 104;
(iii) the 18 or more amino acid modifications comprise the following amino acid modifications at amino acid positions corresponding to the indicated amino acid positions of SEQ ID NO: 104: T19I, del24-26 (LPP), A27S, del69-70 (HV), G142D, V213G, G339D, T376A, D405N, R408S, K417N, N440K, S477N, L452R, T478K, E484A, F486V, Q498R, N501Y, Y505H, D614G, H655Y, N679K, P681H, N764K, D796Y, Q954H, and N969K; or
(iv) a combination thereof.

25. (canceled)

26. (canceled)

27. (canceled)

28. The recombinant protein of claim 1, wherein for (c)
(i) the derivative of the ectodomain comprises: (1) alanine at amino acid residues corresponding to amino acid residues 682 to 685 (RRAR) of the amino acid sequence of SEQ ID NO:104; (2) proline at amino acid residues corresponding to the following amino acid residues of the amino acid sequence of SEQ ID NO: 104: F817, A892, A899, A942, K986, and V987; and (3) two or more of the following amino acid residues at amino acid positions corresponding to the indicated amino acid positions of the amino acid sequence of SEQ ID NO: 104: 440K, 477N, 505H, 679K, 764K, 796Y, 954H, and/or 969K; or
(ii) the ectodomain comprises the amino acid sequence of SEQ ID NO: 103, 35, 85, 47, 59, 91, 97, 19, 21, 23, 41, 53, 65, 71, 79, 33, 39, 45, 51, 57, 63, 69, 77, 83, 89, 95 or 101.

29. (canceled)

30. The recombinant protein of claim 1, wherein the protein further comprises a signal peptide or a signal peptide comprising the amino acid sequence of SEQ ID NO:29.

31. (canceled)

32. The recombinant protein of claim 1, wherein
(i) the recombinant protein further comprises the transmembrane and cytoplasmic domains of Newcastle disease virus (NDV) fusion (F) protein;
(ii) the recombinant protein further comprises a linker and the transmembrane and cytoplasmic domains of Newcastle disease virus (NDV) fusion (F) protein;
(iii) the transmembrane and cytoplasmic domains of Newcastle disease virus (NDV) fusion (F) protein comprises the amino acid sequence of SEQ ID NO: 5; or
(iv) a combination thereof.

33. (canceled)

34. (canceled)

35. A polynucleotide comprising a nucleotide sequence encoding the recombinant protein of claim 1.

36. The polynucleotide of claim 35, wherein the recombinant protein comprises the nucleotide sequence of SEQ ID NO: 34, 84, 102, 90, 96, 46, 58, 18, 20, 22, 40, 52, 64, 70, 78, 32, 82, 100, 88, 94, 44, 56, 38, 50, 62, 68, or 76.

37. A polynucleotide comprising a nucleotide sequence encoding the recombinant protein of claim 32.

38. The polynucleotide of claim 37, wherein the recombinant protein comprises (i) the nucleotide sequence of SEQ ID NO: 30, 42, 54, 66, 80, 86, 92, 98, 6, 7, 10, 11, 14, 15, 36, 48, 60 or 74, or (ii) a nucleotide sequence that encodes the amino acid sequence of SEQ ID NO: 31, 43, 55, 67, 81, 87, 93, 99, 8, 9, 12, 13, 16, 17, 37, 49, 61, or 75.

39. (canceled)

40. (canceled)

41. (canceled)

42. A transgene comprising
(a) a nucleotide sequence encoding a chimeric fusion (F) protein, wherein the chimeric F protein comprises:
(1) an amino acid sequence that is at least 90% identical to SEQ ID NO: 31, 43, 55, 67, 81, 87, 93, 99, 8, 12, 16, 37, 49, 61, or 75 without the signal peptide; or
(2) an amino acid sequence that is at least 90% identical to SEQ ID NO: 31, 43, 55, 67, 81, 87, 93, 99, 8, 12, 16, 37, 49, 61, or 75;
(b) a nucleotide sequence encoding a chimeric fusion (F) protein, wherein the chimeric F protein comprises a derivative of a severe acute respiratory syndrome coronavirus 2 (SARS-CoV-2) Omicron virus spike protein ectodomain and Newcastle disease virus (NDV) F protein transmembrane and cytoplasmic domains, and wherein the derivative of the SARS-CoV-2 Omicron virus spike protein ectodomain comprises:
(1) an amino acid sequence at least 90% identical to the amino acid sequence of SEQ ID NO: 35, 85, 103, 91, 97, 47, 59, 19, 21, 23, 41, 53, 65, 71, or 79; or
(2) an amino acid sequence at least 90% identical to the amino acid sequence of SEQ ID NO: 33, 83, 101, 89, 95, 45, 57, 39, 51, 63, 69, or 77;
(c) a nucleotide sequence that is:
(1) at least 80% identical to SEQ ID NO: 30, 42, 54, 66, 80, 86, 92, 98, 6, 10, 14, 36, 48, 60, or 74 without the nucleotide sequence encoding the signal peptide; or
(2) at least 80% identical to SEQ ID NO: 30, 42, 54, 66, 80, 86, 92, 98, 6, 10, 14, 36, 48, 60, or 74;
(d) a polynucleotide encoding a chimeric fusion (F) protein, wherein the chimeric F protein comprises a derivative of a severe acute respiratory syndrome coronavirus 2 (SARS-CoV-2) Omicron virus spike protein ectodomain and Newcastle disease virus (NDV) F protein transmembrane and cytoplasmic domains, and wherein the nucleotide sequence encoding the derivative of the SARS-CoV-2 Omicron virus spike protein ectodomain comprises:
(1) a nucleotide sequence that is at least 85% identical to the nucleotide sequence of SEQ ID NO: 34, 84, 102, 90, 96, 46, 58, 18, 20, 22, 40, 52, 64, 70, or 78; or
(2) a nucleotide sequence that is at least 85% identical to the nucleotide sequence of SEQ ID NO: 32, 82, 100, 88, 94, 44, 56, 38, 50, 62, 68, or 76;
(e) a ribonucleic acid (RNA) sequence corresponding to the negative sense of the complementary deoxyribonucleic acid (cDNA) sequence of:
(1) a nucleotide sequence that is at least 80% identical to the cDNA sequence of SEQ ID NO: 30, 42, 54, 66, 80, 86, 92, 98, 6, 10, 14, 36, 48, 60, or 74 without the nucleotide sequence encoding the signal peptide; or
(2) a nucleotide sequence that is at least 80% identical to the cDNA sequence of SEQ ID NO: 30, 42, 54, 66, 80, 86, 92, 98, 6, 10, 14, 36, 48, 60, or 74; or
(f) a polynucleotide encoding a chimeric fusion (F) protein, wherein the chimeric F protein comprises a derivative of a Severe acute respiratory syndrome coronavirus 2 (SARS-CoV-2) Omicron virus spike protein ectodomain and Newcastle disease virus (NDV) F protein transmembrane and cytoplasmic domains, and wherein the nucleotide sequence encoding the derivative of the SARS-CoV-2 Omicron virus spike protein ectodomain comprises a ribonucleic acid (RNA) sequence corresponding to the negative sense of the complementary deoxyribonucleic acid (cDNA) sequence of:
(1) a nucleotide sequence that is at least 85% identical to the cDNA sequence of SEQ ID NO: 34, 84, 102, 90, 96, 46, 58, 18, 20, 22, 40, 52, 64, 70, or 78; or
(2) a nucleotide sequence that is at least 85% identical to the cDNA sequence of SEQ ID NO: 32, 82, 100, 88, 94, 44, 56, 38, 50, 62, 68, or 76.

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. The transgene of claim 42, wherein
the SARS-CoV-2 Omicron virus spike protein ectodomain of (a), (d), or (f) is linked via a linker to the Newcastle disease virus (NDV) F protein transmembrane and cytoplasmic domains;
the SARS-CoV-2 Omicron virus spike protein ectodomain of (a), (d), or (f) is linked via a linker to the Newcastle disease virus (NDV) F protein transmembrane and cytoplasmic domains, wherein the linker comprises the amino acid sequence of SEQ ID NO:24;
the transgene of (a), (b), (c), (d), or (f) further comprises a Newcastle disease virus (NDV) gene start sequence;
the transgene of (a), (b), (c), (d), or (f) further comprises a Newcastle disease virus (NDV) gene end sequence;
the transgene of (c) or (d) further comprises the nucleotide sequence of SEQ ID NO:26 and 27;
the transgene of (c) or (d) further comprises the nucleotide sequence of SEQ ID NO: 25, SEQ ID NO: 28, or SEQ ID NOS: 25 and 28;
the transgene of (a), (b), or (f) further comprises an RNA sequence corresponding to the negative sense of the complementary deoxyribonucleic acid (cDNA) sequence of SEQ ID NO: 25, SEQ ID NO:28, or an RNA sequence corresponding to the negative sense of the cDNA sequence of SEQ ID NOS: 25 and 28; or
a combination thereof.

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. A nucleotide sequence comprising the transgene of claim 13, and (1) a Newcastle disease virus (NDV) fusion protein (F) transcription unit, (2) a NDV nucleocapsid protein (NP) transcription unit, (3) a NDV matrix protein (M) transcription unit, (4) a NDV large protein (L) transcription unit, (5) a NDV phosphate protein (P) transcription unit, and (6) a NDV hemagglutinin-neuraminidase (HN) transcription unit.

99. The nucleotide sequence of claim 98, wherein the NDV F transcription unit encodes a NDV F protein comprising a leucine to alanine amino acid substitution at the amino residue corresponding to amino acid residue 289 of the LaSota NDV strain.

100. (canceled)

101. (canceled)

102. A vector comprising the polynucleotide sequence of claim 35.

103. A recombinant Newcastle disease virus (NDV) comprising:
(i) a packaged genome, wherein the packaged genome comprises the transgene of (a), (b), (c), or (d) of claim 13;
(ii) a packaged genome comprising a transgene, wherein the transgene encodes a protein that comprises a derivative of a severe-acute-respiratory-syndrome-related coronavirus 2 (SARS-CoV-2) Omicron virus spike protein ectodomain, wherein the derivative of the SARS-CoV-2 Omicron virus spike protein ectodomain comprises an amino acid sequence at least 90% identical to the amino acid sequence of SEQ ID NO: 35, 85, 103, 91, 97, 47, 59, 19, 21, 23, 41, 53, 65, 71, or 79, preferably comprising the derivative of a SARS-CoV-2 Omicron virus spike protein ectodomain comprises: (1) alanine at amino acid residues corresponding to amino acid residues 682 to 685 (RRAR) of the amino acid sequence of SEQ ID NO: 104; (2) proline at amino acid residues corresponding to the following amino acid residues of the amino acid sequence of SEQ ID NO: 104: F817, A892, A899, A942, K986, and V987; and (3) two or more of the following amino acid residues at amino acid positions corresponding to the indicated amino acid positions of the amino acid sequence of SEQ ID NO: 104: 440K, 477N, 505H, 679K, 764K, 796Y, 954H, and/or 969K;
(iii) a chimeric fusion (F) protein comprising an amino acid sequence that is at least 90% identical to SEQ ID NO: 31, 43, 55, 67, 81, 87, 93, 99, 8, 12, 16, 37, 49, 61, or 75;
(iv) a protein comprising a derivative of a Severe acute respiratory syndrome coronavirus 2 (SARS-CoV-2) Omicron virus spike protein ectodomain comprising an amino acid sequence at least 90% identical to the amino acid sequence of SEQ ID NO: 35, 85, 103, 91, 97, 47, 59, 19, 21, 23, 41, 53, 65, 71, or 79, preferably the derivative of a Severe acute respiratory syndrome coronavirus 2 (SARS-CoV-2) Omicron virus spike protein ectodomain comprises: (1) alanine at amino acid residues corresponding to amino acid residues 682 to 685 (RRAR) of the amino acid sequence of SEQ ID NO: 104; (2) proline at amino acid residues corresponding to the following amino acid residues of the amino acid sequence of SEQ ID NO: 104: F817, A892, A899, A942, K986, and V987; and (3) two or more of the following amino acid residues at amino acid positions corresponding to the indicated amino acid positions of the amino acid sequence of SEQ ID NO: 104: 440K, 477N, 505H, 679K, 764K, 796Y, 954H, and/or 969K; or
(v) a chimeric fusion (F) protein comprising a derivative of a SARS-CoV-2 Omicron virus spike protein ectodomain and NDV F protein transmembrane and cytoplasmic domains, and wherein the derivative of the SARS-CoV-2 Omicron virus spike protein ectodomain comprises an amino acid sequence at least 90% identical to the amino acid sequence of SEQ ID NO: 35, 85, 103, 91, 97, 47, 59, 19, 21, 23, 41, 53, 65, 71, or 79.

104. The recombinant NDV of claim 103, wherein the NDV virion comprises the chimeric fusion (F) protein.

105. (canceled)

106. (canceled)

107. The recombinant NDV of claim 103, wherein for (i) or (ii):
the genome comprises a NDV fusion protein (F) transcription unit, a NDV nucleocapsid protein (NP) transcription unit, a NDV matrix protein (M) transcription unit, a NDV large protein (L) transcription unit, a NDV phosphate protein (P) transcription unit, and a NDV hemagglutinin-neuraminidase (HN) transcription unit, or the genome comprises a NDV fusion protein (F) transcription unit, a NDV nucleocapsid protein (NP) transcription unit, a NDV matrix protein (M) transcription unit, a NDV large protein (L) transcription unit, a NDV phosphate protein (P) transcription unit, and a NDV hemagglutinin-neuraminidase (HN) transcription unit, and wherein the NDV F transcription unit encodes a NDV F protein comprising a leucine to alanine amino acid substitution at the amino residue corresponding to amino acid residue 289 of the LaSota NDV strain;
the transgene is between two NDV transcription units of the packaged genome, such as the NDV phosphate (P) gene and the NDV matrix (M) gene or the NDV nucleocapsid protein (NP) gene and the NDV phosphate (P) gene; or
a combination thereof.

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. (canceled)

120. (canceled)

121. (canceled)

122. The recombinant NDV of claim 103, wherein, for (i), (1) the SARS-CoV-2 Omicron virus spike protein ectodomain is linked via a linker to the NDV F protein transmembrane and cytoplasmic domains, (2) the recombinant NDV comprise an NDV backbone of a lentogenic NDV, LaSota strain, or Hitchner B1 strain, or (3) a combination thereof.

123. (canceled)

124. (canceled)

125. (canceled)

126. (canceled)

127. An immunogenic composition comprising
(a) the recombinant Newcastle disease virus (NDV) of claim 103; and
(b) optionally an adjuvant.

128. (canceled)

129. An immunogenic composition comprising
(a1) the polynucleotide of claim 1, or (a2) a vector comprising the polynucleotide; and
(b) optionally an adjuvant.

130. (canceled)

131. A method for (i) inducing an immune response to Severe acute respiratory syndrome coronavirus 2 (SARS-CoV-2) Omicron spike protein, (ii) preventing Coronavirus Disease 2019 (COVID-19), (iii) immunizing a subject against Severe acute respiratory syndrome coronavirus 2 (SARS-CoV-2), the method comprising administering the immunogenic composition of claim 127 to a subject.

132. (canceled)

133. (canceled)

134. The method of claim 131, wherein
(1) the composition is administered to the subject intranasally or intramuscularly;
(2) the subject to be treated is a human;
(3) the subject has been previously vaccinated with a COVID-19 vaccine;
(4) the subject is administered at least one booster of the immunogenic composition; or
(5) a combination thereof.

135. (canceled)

136. (canceled)

137. (canceled)

138. (canceled)

139. (canceled)

140. A cell line, in vitro cell, or chicken embryonated egg comprising the recombinant NDV of claim 103.

141. A cell line, in vitro cell, or chicken embryonated egg comprising (1) the polynucleotide of claim 35, or (2) a vector comprising the polynucleotide.

142. A cell line, in vitro cell, or chicken embryonated egg expressing the recombinant protein claim 1.

143. (canceled)

144. (canceled)

145. (canceled)

146. (canceled)

147. (canceled)

148. (canceled)

149. (canceled)
